(19) United States
(12) Patent Application Publication
(10) Pub. No.: US 2024/0182984 A1
(43) Pub. Date: Jun. 6, 2024

(54) METHODS FOR ASSESSING PROLIFERATION AND ANTI-FOLATE THERAPEUTIC RESPONSE

(71) Applicants: GeneCentric Therapeutics, Inc., Durham, NC (US); The University of North Carolina at Chapel Hill, Chapel Hill, NC (US)

(72) Inventors: Joel EISNER, Durham, NC (US); Michael MILBURN, Durham, NC (US); Gregory M. MAYHEW, Durham, NC (US); Myla LAI-GOLDMAN, Durham, NC (US); Jianping SUN, Durham, NC (US); Charles M. PEROU, Carrboro, NC (US)

(21) Appl. No.: 18/553,312

(22) PCT Filed: Mar. 30, 2022
(86) PCT No.: PCT/US2022/022618

§ 371 Date: Sep. 29, 2023

Related U.S. Application Data
(60) Provisional application No. 63/167,745, filed on Mar. 30, 2021.

(51) Int. Cl. C12Q 1/6886; A61K 31/517; A61K 31/519; A61P 35/00

(52) U.S. Cl. CPC C12Q 1/6886; A61K 31/517; A61K 31/519; A61P 35/00; C12Q 2600/106; C12Q 2600/158

(57) ABSTRACT

Provided herein is an antifolate predictive response signature for use in determining the response of a subject suffering from cancer to antifolate therapy. Also provided are methods and compositions for determining proliferation in a sample obtained from a subject suffering from cancer through the use of a proliferation gene signature as well as methods for predicting response of a subject suffering from cancer based on an assessment of proliferation in a sample obtained from the subject.

### CROSS-REFERENCE TO RELATED APPLICATIONS

This application claims the benefit of priority to U.S. Provisional Application Ser. No. 63/167,745, filed Mar. 30, 2021, which is herein incorporated by reference in its entirety for all purposes.

### FIELD

The present invention relates to methods for detecting an anti-folate gene expression signature and/or proliferation of cancer cells in a sample obtained from a subject suffering from or suspected of suffering from cancer. The present invention also relates to methods of determining prognosis of a subject suffering from or suspected of suffering from cancer based on said patient's anti-folate gene expression signature and/or detection of the presence or absence of cancer cell proliferation.

### STATEMENT REGARDING SEQUENCE LISTING

The Sequence Listing associated with this application is provided in text format in lieu of a paper copy and is hereby incorporated by reference into the specification. The name of the text file containing the Sequence Listing is GNCN_021_02WO_SeqList_ST25.txt. The text file is 332,527 bytes, and was created on Mar. 28, 2022, and is being submitted electronically via EFS-Web.

### BACKGROUND

### Background

Pemetrexed (LY231514) is a lung cancer drug in the folate analog inhibitor family. Other drugs in this family include methotrexate, trimetrexate, lometrexol, raltitrexed and nolatrexed. Cells are dependent on a full supply of reduced folate to drive a series of 1-carbon reactions that result in synthesis of thymidylate and purines. Antifolates inhibit several enzymes that require this cofactor including synthesis, storage, and transport proteins and have been used in cancer therapy for over 50 years.

Mechanistically, pemetrexed is a multifunctional inhibitor of pathways using folate and its inhibition on multiple targets has been considered a strength of the drug for cancer treatment as compared to other antifolates. Alimta (pemetrexed) is approved for first line treatment of patients with locally advanced or metastatic non-squamous NSCLC in combination with cisplatin as well as first line treatment of patients with metastatic non-squamous NSCLC in combination with platinum chemotherapy and the PD-L1 inhibitor pembrolizumab. It is also approved with cisplatin for treatment of mesothelioma in patients who are not surgery candidates. However, antifolates such as pemetrexed can be highly sensitive to thymidylate synthase levels and higher expression levels can inhibit the drug, which suggest the drug may be more sensitive to cells with decreased levels of these enzymes including thymidylate synthase. Moreover, within some of the approved indications for pemetrexed use, exist subpopulations of patients that appear to respond better to antifolate treatment than others, suggesting that further refinement of patient populations in general as well as within approved indications in order to ascertain which subjects are more likely to be susceptible to antifolate treatment is warranted. The methods, compositions and kits provided herein have been developed to address this need.

### SUMMARY

In one aspect, provided herein is a method of detecting a biomarker in a sample obtained from a patient suffering from cancer, the method comprising measuring the nucleic acid expression level of a plurality of biomarkers selected from Table 1 using an amplification, hybridization and/or sequencing assay. In some cases, the patient was previously diagnosed with a cancer selected from bladder cancer, breast cancer, pancreatic adenocarcinoma, lung adenocarcinoma, lung squamous cell carcinoma, and head and neck adenocarcinoma. In some cases, the amplification, hybridization and/or sequencing assay comprises performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays, Northern blotting, or any other equivalent nucleic acid expression detection techniques. In some cases, the nucleic acid expression level is detected by performing qRT-PCR. In some cases, the detection of the nucleic acid expression level comprises using at least one pair of oligonucleotide primers per each of the plurality of biomarkers selected from Table 1. In some cases, the sample is a formalin-fixed, paraffin-embedded (FFPE) tissue sample, fresh or a frozen tissue sample, an exosome, wash fluids, cell pellets, or a bodily fluid obtained from the patient. In some cases, the bodily fluid is blood or fractions thereof, urine, saliva, or sputum. In some cases, the plurality of biomarkers comprises at least 8 biomarkers, at least 16 biomarkers, at least 24 biomarkers, at least 32 biomarkers, at least 40 biomarkers or at least 48 biomarkers of Table 1. In some cases, the plurality of biomarkers selected from Table 1 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 1. In some cases, the plurality of biomarkers selected from Table 1 comprise flgf, ctsh, sctr, cyp4b1, gpr116, adh1b, cbx7, hlf cep55, tpx2, bub1b, kif4a, ccnb2, kif14, melk, kifl1 or any combination thereof. In some cases, the plurality of biomarkers of Table 1 comprise fgl1, pbk, hspd1, tdg, prc1, dusp4, gtpbp4, zwint, tlr2, cd74, hla-dpb1, hla-dpa1, hla-dra, itgb2, fas, hla-drb1, plau, gbp1, dse, ccdc109b, tgfbi, cxcl10, lgals1, tubb6, gjb1, rap1gap, cacna2d2, selenbp1, tfcp2l1, sorbs2, unc13b, tacc2 or any combination thereof. In some cases, the plurality of biomarkers comprises all the classifier biomarkers of Table 1.

In another aspect, provided herein is a method of detecting a biomarker in a sample obtained from a patient suffering from cancer, the method consisting essentially of measuring the nucleic acid expression level of a plurality of biomarkers selected from Table 1 using an amplification, hybridization and/or sequencing assay. In some cases, the patient was previously diagnosed with a cancer selected from bladder cancer, breast cancer, pancreatic adenocarcinoma, lung adenocarcinoma, lung squamous cell carcinoma, and head and neck adenocarcinoma. In some cases, the amplification, hybridization and/or sequencing assay comprises performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays, Northern blotting, or any other equivalent gene expression detection techniques. In some cases, the nucleic acid expression level is detected by performing qRT-PCR. In some cases, the detection of the nucleic acid expression level comprises using at least one pair of oligonucleotide primers per each of the plurality of biomarkers selected from Table 1. In some cases, the sample is a formalin-fixed, paraffin-embedded (FFPE) tissue sample, fresh or a frozen tissue sample, an exosome, wash fluids, cell pellets, or a bodily fluid obtained from the patient. In some cases, the bodily fluid is blood or fractions thereof, urine, saliva, or sputum. In some cases, the plurality of biomarkers consists essentially of at least 8 biomarkers, at least 16 biomarkers, at least 24 biomarkers, at least 32 biomarkers, at least 40 biomarkers or at least 48 biomarkers of Table 1. In some cases, the plurality of biomarkers selected from Table 1 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 1. In some cases, the plurality of biomarkers selected from Table 1 comprise flgf, ctsh, sctr, cyp4b1, gpr116, adh1b, cbx7, hlf cep55, tpx2, bub1b, kif4a, ccnb2, kif14, melk, kif11 or any combination thereof. In some cases, the plurality of biomarkers of Table 1 comprise fgl1, pbk, hspd1, tdg, prc1, dusp4, gtpbp4, zwint, tlr2, cd74, hla-dpb1, hla-dpa1, hla-dra, itgb2, fas, hla-drb1, plau, gbp1, dse, ccdc109b, tgfbi, cxcl10, lgals1, tubb6, gjb1, rap1gap, cacna2d2, selenbp1, tfcp2l1, sorbs2, unc13b, tacc2 or any combination thereof. In some cases, the plurality of biomarkers consists essentially of all the biomarkers of Table 1.

In yet another aspect, provided herein is a method of detecting a biomarker in a sample obtained from a patient suffering from cancer, the method consisting of measuring the nucleic acid expression level of a plurality of biomarkers selected from Table 1 using an amplification, hybridization and/or sequencing assay. In some cases, the patient was previously diagnosed with a cancer selected from bladder cancer, breast cancer, pancreatic adenocarcinoma, lung adenocarcinoma, lung squamous cell carcinoma, and head and neck adenocarcinoma. In some cases, the amplification, hybridization and/or sequencing assay comprises performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays, Northern blotting, or any other equivalent gene expression detection techniques. In some cases, the nucleic acid expression level is detected by performing qRT-PCR. In some cases, the detection of the nucleic acid expression level comprises using at least one pair of oligonucleotide primers per each of the plurality of biomarkers selected from Table 1. In some cases, the sample is a formalin-fixed, paraffin-embedded (FFPE) tissue sample, fresh or a frozen tissue sample, an exosome, wash fluids, cell pellets, or a bodily fluid obtained from the patient. In some cases, the bodily fluid is blood or fractions thereof, urine, saliva, or sputum. In some cases, the plurality of biomarkers consists of at least 8 biomarkers, at least 16 biomarkers, at least 24 biomarkers, at least 32 biomarkers, at least 40 biomarkers or at least 48 biomarkers of Table 1. In some cases, the plurality of biomarkers selected from Table 1 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 1. In some cases, the plurality of biomarkers selected from Table 1 comprise flgf, ctsh, sctr, cyp4b1, gpr116, adh1b, cbx7, hlf cep55, tpx2, bub1b, kif4a, ccnb2, kif14, melk, kif11 or any combination thereof. In some cases, the plurality of biomarkers of Table 1 comprise fgl1, pbk, hspd1, tdg, prc1, dusp4, gtpbp4, zwint, tlr2, cd74, hla-dpb1, hla-dpa1, hla-dra, itgb2, fas, hla-drb1, plau, gbp1, dse, ccdc109b, tgfbi, cxcl10, lgals1, tubb6, gjb1, rap1gap, cacna2d2, selenbp1, tfcp2l1, sorbs2, unc3b, tacc2 or any combination thereof. In some cases, the plurality of biomarkers comprises, consists essentially of or consists of all the biomarkers of Table 1.

In one aspect, provided herein is a method of determining whether a patient suffering from cancer is likely to respond to treatment with an antifolate agent, the method comprising, determining an antifolate predictive response signature of a sample obtained from a patient suffering from cancer; and based on the antifolate predictive response signature, assessing whether the patient is likely to respond to treatment with an antifolate agent, wherein a positive antifolate predictive response signature predicts that the patient is likely to respond to the treatment with an antifolate agent. In some cases, the anti-folate agent is selected from pemetrexed, methotrexate, trimetrexate, lometrexol, raltitrexed and nolatrexed. In some cases, the antifolate agent is pemetrexed. In some cases, the antifolate agent is raltitrexed. In some cases, the cancer the patient is suffering from is selected from bladder cancer, breast cancer, pancreatic adenocarcinoma, lung adenocarcinoma, lung squamous cell carcinoma, and head and neck adenocarcinoma. In some cases, the sample is a formalin-fixed, paraffin-embedded (FFPE) tissue sample, fresh or a frozen tissue sample, an exosome, or a bodily fluid obtained from the patient. In some cases, the bodily fluid is blood or fractions thereof, urine, saliva, or sputum. In some cases, the determining the antifolate predictive response signature of the sample obtained from the patient suffering from cancer comprises determining expression levels of a plurality of classifier biomarkers. In some cases, the determining the expression levels of the plurality of classifier biomarkers is at a nucleic acid level by performing RNA sequencing, reverse transcriptase polymerase chain reaction (RT-PCR) or hybridization-based analyses. In some cases, the plurality of classifier biomarkers for determining the antifolate predictive response signature is selected from Table 1. In some cases, the RT-PCR is quantitative real time reverse transcriptase polymerase chain reaction (qRT-PCR). In some cases, the RT-PCR is performed with primers specific to the classifier biomarkers selected from the plurality of classifier biomarkers of Table 1. In some cases, the method further comprises comparing the detected levels of expression of the plurality of classifier biomarkers of Table 1 to the expression of the plurality of classifier biomarkers of Table 1 in at least one sample training set(s), wherein the at least one sample training set comprises expression data of the plurality of classifier biomarkers of Table 1 from a reference adenocarcinoma TRU (bronchioid) sample, expression data of the plurality of classifier biomarkers of Table 1 from a reference adenocarcinoma PP (magnoid) sample, expression data of the plurality of classifier biomarkers of Table 1 from a reference adenocarcinoma PI (squamoid) sample, or a combination thereof; and classifying the sample as TRU, PP, or PI based on the results of the comparing step. In some cases, the comparing step comprises applying a statistical algorithm which comprises determining a correlation between the expression data obtained from the sample and the expression data from the at least one training set(s); and classifying the sample as a TRU, PP, or PI subtype based on the results of the statistical algorithm. In some cases, the TRU subtype is indicative of a positive antifolate predictive response signature, wherein the positive antifolate predictive response signature selects the patient for treatment with an antifolate agent. In some cases, the plurality of classifier biomarkers comprises at least 8 biomarker nucleic acids, at least 16 biomarker nucleic acids, at least 24 biomarker nucleic acids, at least 32 biomarker nucleic acids, at least 140 biomarker nucleic acids or all 48 biomarker nucleic acids of Table 1. In some cases, the plurality of classifier biomarkers selected from Table 1 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the classifier biomarkers from Table 1. In some cases, the plurality of classifier biomarkers selected from Table 1 comprise flgf, ctsh, sctr, cyp4b1, gpr116, adh1b, cbx7, hlf cep55, tpx2, bub1b, kif4a, ccnb2, kif14, melk, kif11 or any combination thereof. In some cases, the plurality of classifier biomarkers of Table 1 comprise fgl1, pbk, hspd1, tdg, prc1, dusp4, gtpbp4, zwint, tlr2, cd74, hla-dpb1, hla-dpa1, hla-dra, itgb2, fas, hla-drb1, plau, gbp1, dse, ccdc109b, tgfbi, cxcl10, lgals1, tubb6, gjb1, rap1gap, cacna2d2, selenbp1, tfcp2l1, sorbs2, unc13b, tacc2 or any combination thereof. In some cases, the method further comprises determining the expression level of one or more anti-folate drug targets in the sample obtained from the patient. In some cases, the one or more anti-folate drug targets is selected from dhfr, gart, tyms, atic, or mthfd1l genes. In some cases, the method further comprises determining a tumor mutational burden of the tumor sample obtained from the patient. In some cases, the method further comprises determining a proliferation signature of the tumor sample obtained from the patient. In some cases, the determining the proliferation signature in the tumor sample obtained from a patient comprises measuring a nucleic acid expression level in the sample of at least five classifier genes from a plurality of classifier genes, wherein the plurality of classifier genes consists of only targeting protein for Xklp2 (TPX2), discs large homolog associated protein 5 (DLGAP5), Holliday junction recognition protein (HJURP), kinesin family member 4A (KIF4A), kinesin family member 2C (KIF2C), polo like kinase 1 (PLK1), maternal embryonic leucine zipper kinase (MELK), Cyclin B2 (CCNB2), budding uninhibited by benzimidazoles 1 (BUB1), kinesin family member 23 (KIF23), ubiquitin conjugating enzyme E2 C (UBE2C), kinesin family member 20A (KIF20A), trophinin associated protein (TROAP), aurora kinase B (AURKB), ribonucleotide reductase regulatory subunit M2 (RRM2), MYB proto-oncogene like 2 (MYBL2), antigen KI-67 (MK167), cell division cycle 20 (CDC20), centrosomal protein 55 (CEP55), topoisomerase 2-alpha (TOP2A), baculoviral IAP repeat containing 5 (BIRC5), abnormal spindle microtubule assembly (ASPM), extra spindle pole bodies like 1, separase (ESPL1), kinesin family member 18B (KIF18B), IQ motif containing GTPase activating protein 3 (IQGAP3), and effector cell protease receptor-1 (EPR1), wherein the nucleic acid expression level of the at least five classifier genes represents a proliferation signature. In some cases, the nucleic acid expression level is measured using an amplification, sequencing or hybridization assay. In some cases, the amplification, hybridization and/or sequencing assay comprises performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays, Northern blotting, nCounter DX Analysis System or any other equivalent gene expression detection techniques. In some cases, the expression level is detected by performing RNA-seq. In some cases, the measuring the nucleic acid expression level is for at least 10, 15, 20 or 25 classifier genes from the plurality of classifier genes. In some cases, the measuring the nucleic acid expression level is for all of the classifier genes from the plurality of classifier genes. In some cases, the method further comprises determining a proliferation score, wherein the determining the proliferation score comprises determining a mean nucleic acid expression level for the at least five classifier biomarkers from the plurality of classifier biomarkers. In some cases, the method further comprises determining a level and/or activity of at least one additional marker involved in cell proliferation and mitosis. In some cases, the at least one additional marker is Ki67 or CD31.

In another aspect, provided herein is a method for selecting a patient suffering from cancer for an antifolate agent, the method comprising, determining an antifolate predictive response signature of a sample obtained from a patient suffering from cancer; and selecting the patient for treatment with an antifolate agent if the antifolate response signature is positive. In some cases, the anti-folate agent is selected from pemetrexed, methotrexate, trimetrexate, lometrexol, raltitrexed and nolatrexed. In some cases, the antifolate agent is pemetrexed. In some cases, the antifolate agent is raltitrexed. In some cases, the cancer the patient is suffering from is selected from bladder cancer, breast cancer, pancreatic adenocarcinoma, lung adenocarcinoma, lung squamous cell carcinoma, and head and neck adenocarcinoma. In some cases, the sample is a formalin-fixed, paraffin-embedded (FFPE) tissue sample, fresh or a frozen tissue sample, an exosome, or a bodily fluid obtained from the patient. In some cases, the bodily fluid is blood or fractions thereof, urine, saliva, or sputum. In some cases, the determining the antifolate predictive response signature of the sample obtained from the patient suffering from cancer comprises determining expression levels of a plurality of classifier biomarkers. In some cases, the determining the expression levels of the plurality of classifier biomarkers is at a nucleic acid level by performing RNA sequencing, reverse transcriptase polymerase chain reaction (RT-PCR) or hybridization-based analyses. In some cases, the plurality of classifier biomarkers for determining the antifolate predictive response signature is selected from Table 1. In some cases, the RT-PCR is quantitative real time reverse transcriptase polymerase chain reaction (qRT-PCR). In some cases, the RT-PCR is performed with primers specific to the classifier biomarkers selected from the plurality of classifier biomarkers of Table 1. In some cases, the method further comprises comparing the detected levels of expression of the plurality of classifier biomarkers of Table 1 to the expression of the plurality of classifier biomarkers of Table 1 in at least one sample training set(s), wherein the at least one sample training set comprises expression data of the plurality of classifier biomarkers of Table 1 from a reference adenocarcinoma TRU (bronchioid) sample, expression data of the plurality of classifier biomarkers of Table 1 from a reference adenocarcinoma PP (magnoid) sample, expression data of the plurality of classifier biomarkers of Table 1 from a reference adenocarcinoma PI (squamoid) sample, or a combination thereof, and classifying the sample as TRU, PP, or PI based on the results of the comparing step. In some cases, the comparing step comprises applying a statistical algorithm which comprises determining a correlation between the expression data obtained from the sample and the expression data from the at least one training set(s); and classifying the sample as a TRU, PP, or PI subtype based on the results of the statistical algorithm. In some cases, the TRU subtype is indicative of a positive antifolate predictive response signature, wherein the positive antifolate predictive response signature selects the patient for treatment with an antifolate agent. In some cases, the plurality of classifier biomarkers comprises at least 8 biomarker nucleic acids, at least 16 biomarker nucleic acids, at least 24 biomarker nucleic acids, at least 32 biomarker nucleic acids, at least 140 biomarker nucleic acids or all 48 biomarker nucleic acids of Table 1. In some cases, the plurality of classifier biomarkers selected from Table 1 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the classifier biomarkers from Table 1. In some cases, the plurality of classifier biomarkers selected from Table 1 comprise flgf, ctsh, sctr, cyp4b1, gpr116, adh1b, cbx7, hlf cep55, tpx2, bub1b, kif4a, ccnb2, kif14, melk, kif11 or any combination thereof. In some cases, the plurality of classifier biomarkers of Table 1 comprise fgl1, pbk, hspd1, tdg, prc1, dusp4, gtpbp4, zwint, tlr2, cd74, hla-dpb1, hla-dpa1, hla-dra, itgb2, fas, hla-drb1, plau, gbp1, dse, ccdc109b, tgfbi, cxcl10, lgals1, tubb6, gjb1, rap1gap, cacna2d2, selenbp1, tfcp2l1, sorbs2, unc13b, tacc2 or any combination thereof. In some cases, the method further comprises determining the expression level of one or more anti-folate drug targets in the sample obtained from the patient. In some cases, the one or more anti-folate drug targets is selected from dhfr, gart, tyms, atic, or mthfd1l genes. In some cases, the method further comprises determining a tumor mutational burden of the tumor sample obtained from the patient. In some cases, the method further comprises determining a proliferation signature of the tumor sample obtained from the patient. In some cases, the determining the proliferation signature in the tumor sample obtained from a patient comprises measuring a nucleic acid expression level in the sample of at least five classifier genes from a plurality of classifier genes, wherein the plurality of classifier genes consists of only targeting protein for Xklp2 (TPX2), discs large homolog associated protein 5 (DLGAP5), Holliday junction recognition protein (HJURP), kinesin family member 4A (KIF4A), kinesin family member 2C (KIF2C), polo like kinase 1 (PLK1), maternal embryonic leucine zipper kinase (MELK), Cyclin B2 (CCNB2), budding uninhibited by benzimidazoles 1 (BUB1), kinesin family member 23 (KIF23), ubiquitin conjugating enzyme E2 C (UBE2C), kinesin family member 20A (KIF20A), trophinin associated protein (TROAP), aurora kinase B (AURKB), ribonucleotide reductase regulatory subunit M2 (RRM2), MYB proto-oncogene like 2 (MYBL2), antigen KI-67 (MK167), cell division cycle 20 (CDC20), centrosomal protein 55 (CEP55), topoisomerase 2-alpha (TOP2A), baculoviral IAP repeat containing 5 (BIRC5), abnormal spindle microtubule assembly (ASPM), extra spindle pole bodies like 1, separase (ESPL1), kinesin family member 18B (KIF18B), IQ motif containing GTPase activating protein 3 (IQGAP3), and effector cell protease receptor-1 (EPR1), wherein the nucleic acid expression level of the at least five classifier genes represents a proliferation signature. In some cases, the nucleic acid expression level is measured using an amplification, sequencing or hybridization assay. In some cases, the amplification, hybridization and/or sequencing assay comprises performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays, Northern blotting, nCounter DX Analysis System or any other equivalent gene expression detection techniques. In some cases, the expression level is detected by performing RNA-seq. In some cases, the measuring the nucleic acid expression level is for at least 10, 15, 20 or 25 classifier genes from the plurality of classifier genes. In some cases, the measuring the nucleic acid expression level is for all of the classifier genes from the plurality of classifier genes. In some cases, the method further comprises determining a proliferation score, wherein the determining the proliferation score comprises determining a mean nucleic acid expression level for the at least five classifier biomarkers from the plurality of classifier biomarkers. In some cases, the method further comprises determining a level and/or activity of at least one additional marker involved in cell proliferation and mitosis. In some cases, the at least one additional marker is Ki67 or CD31.

In still another aspect, provided herein is a method of treating cancer in a patient, the method comprising: measuring the expression level of a plurality of classifier biomarkers in a sample obtained from a patient suffering from cancer, wherein the plurality of classifier biomarkers are selected from a set of classifier biomarkers listed in Table 1, wherein the measured expression levels of the plurality of classifier biomarkers provide an antifolate predictive response signature for the sample; and administering an antifolate agent based on presence of a positive antifolate predictive response signature. In some cases, the anti-folate agent is selected from pemetrexed, methotrexate, trimetrexate, lometrexol, raltitrexed and nolatrexed. In some cases, the antifolate agent is pemetrexed. In some cases, the antifolate agent is raltitrexed. In some cases, the cancer is selected from bladder cancer, breast cancer, pancreatic adenocarcinoma, lung adenocarcinoma, lung squamous cell carcinoma, and head and neck adenocarcinoma, In some cases, the sample is a formalin-fixed, paraffin-embedded (FFPE) tissue sample, fresh or a frozen tissue sample, an exosome, or a bodily fluid obtained from the patient. In some cases, the bodily fluid is blood or fractions thereof, urine, saliva, or sputum. In some cases, the measuring the expression levels of the plurality of classifier biomarkers is at a nucleic acid level by performing RNA sequencing, reverse transcriptase polymerase chain reaction (RT-PCR) or hybridization-based analyses. In some cases, the RT-PCR is quantitative real time reverse transcriptase polymerase chain reaction (qRT-PCR). In some cases, the RT-PCR is performed with primers specific to the classifier biomarkers selected from the plurality of classifier biomarkers of Table 1. In some cases, the method further comprises comparing the detected levels of expression of the plurality of classifier biomarkers of Table 1 to the expression of the plurality of classifier biomarkers of Table 1 in at least one sample training set(s), wherein the at least one sample training set comprises expression data of the plurality of classifier biomarkers of Table 1 from a reference adenocarcinoma TRU (bronchioid) sample, expression data of the plurality of classifier biomarkers of Table 1 from a reference adenocarcinoma PP (magnoid) sample, expression data of the plurality of classifier biomarkers of Table 1 from a reference adenocarcinoma PI (squamoid) sample, or a combination thereof, and classifying the sample as TRU, PP, or PI based on the results of the comparing step. In some cases, the comparing step comprises applying a statistical algorithm which comprises determining a correlation between the expression data obtained from the sample and the expression data from the at least one training set(s); and classifying the sample as a TRU, PP, or PI subtype based on the results of the statistical algorithm. In some cases, the TRU subtype is indicative of the positive antifolate predictive response signature. In some cases, the plurality of classifier biomarkers comprises at least 8 biomarkers, at least 16 classifier biomarkers, at least 24 classifier biomarkers, at least 32 classifier biomarkers, at least 40 classifier biomarkers, or all 48 classifier biomarkers of Table 1. In some cases, the method further comprises determining the expression level of one or more anti-folate drug targets in the sample obtained from the patient. In some cases, the one or more anti-folate drug targets is selected from dhfr, gart, tyms, atic, or mthfd1l genes. In some cases, the method further comprises determining a tumor mutational burden of the sample obtained from the patient. In some cases, the method further comprises determining a proliferation signature of the sample obtained from the patient. In some cases, the determining the proliferation signature in the sample obtained from the patient comprises measuring a nucleic acid expression level in the sample of at least five classifier genes from a plurality of classifier genes, wherein the plurality of classifier genes consists of only targeting protein for Xklp2 (TPX2), discs large homolog associated protein 5 (DLGAP5), Holliday junction recognition protein (HJURP), kinesin family member 4A (KIF4A), kinesin family member 2C (KIF2C), polo like kinase 1 (PLK1), maternal embryonic leucine zipper kinase (MELK), Cyclin B2 (CCNB2), budding uninhibited by benzimidazoles 1 (BUB1), kinesin family member 23 (KIF23), ubiquitin conjugating enzyme E2 C (UBE2C), kinesin family member 20A (KIF20A), trophinin associated protein (TROAP), aurora kinase B (AURKB), ribonucleotide reductase regulatory subunit M2 (RRM2), MYB proto-oncogene like 2 (MYBL2), antigen KI-67 (MK167), cell division cycle 20 (CDC20), centrosomal protein 55 (CEP55), topoisomerase 2-alpha (TOP2A), baculoviral IAP repeat containing 5 (BIRC5), abnormal spindle microtubule assembly (ASPM), extra spindle pole bodies like 1, separase (ESPL1), kinesin family member 18B (KIF18B), IQ motif containing GTPase activating protein 3 (IQGAP3), and effector cell protease receptor-1 (EPR1), wherein the expression level of nucleic acid of the at least five classifier genes represents a proliferation signature. In some cases, the nucleic acid expression level is measured using an amplification, sequencing or hybridization assay. In some cases, the amplification, hybridization and/or sequencing assay comprises performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays, Northern blotting, nCounter DX Analysis System or any other equivalent gene expression detection techniques. In some cases, the nucleic acid expression level is detected by performing RNA-seq. In some cases, the measuring the nucleic acid expression level is for at least 10, 15, 20 or 25 classifier genes from the plurality of classifier genes. In some cases, the measuring the nucleic acid expression level is for all of the classifier genes from the plurality of classifier genes. In some cases, the method further comprises determining a proliferation score, wherein the determining the proliferation score comprises determining a mean nucleic acid expression level for the at least five classifier biomarkers from the plurality of classifier biomarkers. In some cases, the method further comprises determining a level and/or activity of at least one additional marker involved in cell proliferation and mitosis. In some cases, the at least one additional marker is Ki67 or CD31.

In one aspect, provided herein is a method of detecting a proliferation signature in a sample obtained from a subject, the method comprising measuring a nucleic acid expression level of at least five classifier genes from a plurality of classifier genes in the sample, wherein the plurality of classifier genes consists of only targeting protein for Xklp2 (TPX2), discs large homolog associated protein 5 (DLGAP5), Holliday junction recognition protein (HJURP), kinesin family member 4A (KIF4A), kinesin family member 2C (KIF2C), polo like kinase 1 (PLK1), maternal embryonic leucine zipper kinase (MELK), Cyclin B2 (CCNB2), budding uninhibited by benzimidazoles 1 (BUB1), kinesin family member 23 (KIF23), ubiquitin conjugating enzyme E2 C (UBE2C), kinesin family member 20A (KIF20A), trophinin associated protein (TROAP), aurora kinase B (AURKB), ribonucleotide reductase regulatory subunit M2 (RRM2), MYB proto-oncogene like 2 (MYBL2), antigen KI-67 (MK167), cell division cycle 20 (CDC20), centrosomal protein 55 (CEP55), topoisomerase 2-alpha (TOP2A), baculoviral IAP repeat containing 5 (BIRC5), abnormal spindle microtubule assembly (ASPM), extra spindle pole bodies like 1, separase (ESPL1), kinesin family member 18B (KIF18B), IQ motif containing GTPase activating protein 3 (IQGAP3), and effector cell protease receptor-1 (EPR1), wherein the nucleic acid expression level of the at least five classifier genes represents a proliferation signature. In some cases, the subject is suffering from or suspected of suffering from Cervical Kidney renal papillary cell carcinoma (KIRP), Breast Invasive Carcinoma (BRCA), Thyroid Cancer (THCA), Bladder Carcinoma (BLCA), Prostate Adenocarcinoma (PRAD), Kidney Chromophobe (KICH), Cervical Squamous Cell Carcinoma and Endocervical Adenocarcinoma (CESC), Kidney Renal Clear Cell Carcinoma (KIRC), Liver Hepatocellular Carcinoma (LIHC), Low Grade Glioma (LGG), Sarcoma (SARC), Lung Adenocarcinoma (LUAD), Colon Adenocarcinoma (COAD), Head-Neck Squamous Cell Carcinoma (HNSC), Uterine Corpus Endometrial Carcinoma (UCEC), Glioblastoma Multiforme (GBM), Esophageal Carcinoma (ESCA), Stomach Adenocarcinoma (STAD), Ovarian Cancer (OV), and Rectum Adenocarcinoma (READ). In some cases, the sample is a formalin-fixed, paraffin-embedded (FFPE) tissue sample, fresh or a frozen tissue sample, an exosome, wash fluids, cell pellets, or a bodily fluid obtained from the subject. In some cases, the sample is an FFPE tissue sample. In some cases, the sample is a fresh frozen tissue sample. In some cases, the nucleic acid expression level is measured using an amplification, sequencing or hybridization assay. In some cases, the amplification, hybridization and/or sequencing assay comprises performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays, Northern blotting, nCounter DX Analysis System or any other equivalent gene expression detection techniques. In some cases, the nucleic acid expression level is detected by performing RNA-seq. In some cases, the measuring the nucleic acid expression level is for at least 10, 15, 20 or 25 classifier genes from the plurality of classifier genes. In some cases, the measuring the nucleic acid expression level is for all of the classifier genes from the plurality of classifier genes. In some cases, the method further comprises determining a proliferation score, wherein the determining the proliferation score comprises determining a mean nucleic acid expression level for the at least five classifier biomarkers from the plurality of classifier biomarkers. In some cases, the method further comprises determining a level and/or activity of at least one additional marker involved in cell proliferation and mitosis. In some cases, the at least one additional marker is Ki67 or CD31.

In another aspect, provided herein is a method of determining metastatic disease in a subject, the method comprising: measuring a nucleic acid expression level of at least five classifier genes from a plurality of classifier genes in a first sample obtained from the subject, wherein the plurality of classifier genes consists of only tpx2, dlgap5, hjurp, kif4a, kif2c, plk1, melk, ccnb2, bub1, kif23, ube2c, kif20a, troap, aurkb, rrm2, mybl2, mki67, cdc20, cep55, top2a, birc5, aspm, espl1, kif18b, iqgap3 and epr1, wherein the nucleic acid expression level of the at least five classifier genes represents a proliferation signature of the first sample; measuring the nucleic acid expression level of the same at least five classifier genes from the plurality of classifier genes in a second sample, wherein the nucleic acid expression level of the at least five classifier genes represents a proliferation signature of the second sample; and determining existence of a correlation between the proliferation signature of the first sample and the proliferation signature of the second sample, wherein the existence of a correlation is indicative of the likelihood of metastatic disease in the subject. In some cases, the second sample is obtained from the subject, wherein the first and second samples are obtained from different regions of the subject's body. In some cases, the second sample is obtained from a control subject that does not have metastatic disease, wherein the second sample is obtained from the same area of the body as the first sample. In some cases, the subject is suffering from or suspected of suffering from KIRP, BRCA, THCA, BLCA, PRAD, KICH, CESC, KIRC, LIHC, LGG, SARC, LUAD, COAD, HNSC, UCEC, GBLM, ESCA, STAD, OV and READ. In some cases, the first and/or second sample is a formalin-fixed, paraffin-embedded (FFPE) tissue sample, fresh or a frozen tissue sample, an exosome, wash fluids, cell pellets, or a bodily fluid obtained from the subject. In some cases, the first sample and the second sample is an FFPE tissue sample. In some cases, the first sample and the second sample is a fresh frozen tissue sample. In some cases, the nucleic acid expression level is measured using an amplification, sequencing or hybridization assay. In some cases, the amplification, hybridization and/or sequencing assay comprises performing quantitative real time reverse transcriptase polymerase chain reaction (qRT-PCR), RNA-seq, microarrays, gene chips, nCounter Gene Expression Assay, Serial Analysis of Gene Expression (SAGE), Rapid Analysis of Gene Expression (RAGE), nuclease protection assays, Northern blotting, nCounter DX Analysis System or any other equivalent gene expression detection techniques. In some cases, the nucleic acid expression level is detected by performing RNA-seq. In some cases, the measuring the nucleic acid expression level is for at least 10, 15, 20 or 25 classifier genes from the plurality of classifier genes. In some cases, the measuring the nucleic acid expression level is for all of the classifier genes from the plurality of classifier genes. In some cases, the method further comprises determining a level and/or activity of at least one additional marker involved in cell proliferation and mitosis. In some cases, the at least one additional marker is Ki67 or CD31. In some cases, the determining the existence of a correlation comprises applying a statistical algorithm to the proliferation signature of the first sample and the proliferation signature of the second sample. In some cases, prior to determining the existence of a correlation between the proliferation signature of the first sample and the proliferation signature of the second sample, determining a proliferation score for the first sample and the second sample, wherein the determining the proliferation score comprises determining a mean nucleic acid expression level across the at least five classifier biomarkers from the plurality of classifier biomarkers for the first sample and the second sample, whereby the determining the existence of a correlation entails determining the existence of a correlation between the proliferation score of the first sample and the proliferation score of the second sample. In some cases, the determining the existence of a correlation comprises applying a statistical algorithm to the proliferation score of the first sample and the proliferation score of the second sample.

In still another aspect, provided herein is a method of treating a subject suffering from or suspected of suffering from cancer, the method comprising: (a) determining a proliferation score of a sample obtained from the subject, wherein the determining the proliferation score comprises: (i) measuring a nucleic acid expression level of at least five classifier genes from a plurality of classifier genes in the sample obtained from the subject, wherein the plurality of classifier genes consists of only tpx2, dlgap5, hjurp, kif4a, kif2c, plk1, melk, ccnb2, bub1, kif23, ube2c, kif20a, troap, aurkb, rrm2, mybl2, mki67, cdc20, cep55, top2a, birc5, aspm, espl1, kif18b, iqgap3 and epr1; and (ii) calculating a mean nucleic acid expression level across the at least five classifier biomarkers from the plurality of classifier biomarkers, wherein the mean nucleic acid expression level across the at least five classifier biomarkers from the plurality of classifier biomarkers represents the proliferation score; (b) determining a proliferation score of a control sample, wherein the determining the proliferation score comprises: (i) measuring a nucleic acid expression level of at least five classifier genes from a plurality of classifier genes in the control sample, wherein the plurality of classifier genes consists of only tpx2, dlgap5, hjurp, kif4a, kif2c, plk1, melk, ccnb2, bub1, kif23, ube2c, kif20a, troap, aurkb, rrm2, mybl2, mki67, cdc20, cep55, top2a, birc5, aspm, espl1, kif18b, iqgap3 and epr1; and (ii) calculating a mean nucleic acid expression level across the at least five classifier biomarkers from the plurality of classifier biomarkers, wherein the mean nucleic acid expression level across the at least five classifier biomarkers from the plurality of classifier biomarkers represents the proliferation score; (c) comparing the proliferation score of the sample obtained from the subject to the proliferation score of the control sample; and (d) administering a therapeutic agent to the subject based on the comparison between the proliferation score of the sample obtained from the subject and the control sample, thereby treating the cancer. In some cases, the control sample is from a healthy subject. In some cases, the control sample is a non-proliferative cancer sample. In some cases, the comparison shows an increased proliferation score of the sample obtained from the subject and the therapeutic agent administered is tailored to proliferative cancers. In some cases, the therapeutic agent is selected from radiation therapy and anti-angiogenic therapeutic agents. In some cases, the cancer is selected from KIRP, BRCA, THCA, BLCA, PRAD, KICH, CESC, KIRC, LIHC, LGG, SARC, LUAD, COAD, HNSC, UCEC, GBM, ESCA, STAD, OV and READ. In some cases, the sample obtained from the subject and/or the control sample is a formalin-fixed, paraffin-embedded (FFPE) tissue sample, fresh or a frozen tissue sample, an exosome, wash fluids, cell pellets, or a bodily fluid obtained from the patient. In some cases, the sample obtained from the subject and/or the control is an FFPE tissue sample. In some cases, the sample obtained from the subject and/or the control is a fresh frozen tissue sample. In some cases, the nucleic acid expression level is measured using an amplification, sequencing or hybridization assay. In some cases, the amplification, hybridization and/or sequencing assay comprises performing quantitative real time reverse transcriptase polymerase chain reaction (qRT-PCR), RNA-seq, microarrays, gene chips, nCounter Gene Expression Assay, Serial Analysis of Gene Expression (SAGE), Rapid Analysis of Gene Expression (RAGE), nuclease protection assays, Northern blotting, nCounter DX Analysis System or any other equivalent gene expression detection techniques. In some cases, the nucleic acid expression level is detected by performing RNA-seq. In some cases, the measuring the nucleic acid expression level is for at least 10, 15, 20 or 25 classifier genes from the plurality of classifier genes. In some cases, the measuring the nucleic acid expression level is for all of the classifier genes from the plurality of classifier genes. In some cases, the method further comprises determining a level and/or activity of at least one additional marker involved in cell proliferation and mitosis. In some cases, the at least one additional marker is Ki67 or CD31. In some cases, the method further comprises determining a subtype of the sample obtained from the subject prior to administering the therapeutic agent and administering the therapeutic agent to the subject based on the comparison between the proliferation score of the sample obtained from the subject and the control sample and the subtype of the sample obtained from the subject. In some cases, the determining the subtype is performed by histological examination of the sample. In some cases, the determining the subtype is performed by gene expression analysis of the sample. In some cases, the gene expression analysis of the sample is performed using a gene expression sub-typer that is publicly available.

In another aspect, provided herein is a method of determining a disease outcome in a subject suffering from or suspected of suffering from cancer, the method comprising: (a) determining a proliferation score of a sample obtained from the subject, wherein the determining the proliferation score comprises: (i) measuring a nucleic acid expression level of at least five classifier genes from a plurality of classifier genes in the sample obtained from the cancer patient, wherein the plurality of classifier genes consists of only tpx2, dlgap5, hjurp, kif4a, kif2c, plk1, melk, ccnb2, bub1, kif23, ube2c, kif20a, troap, aurkb, rrm2, mybl2, mki67, cdc20, cep55, top2a, birc5, aspm, espl1, kif18b, iqgap3 and epr1; and (ii) calculating a mean nucleic acid expression level across the at least five classifier biomarkers from the plurality of classifier biomarkers, wherein the mean nucleic acid expression level across the at least five classifier biomarkers from the plurality of classifier biomarkers represents the proliferation score; (b) determining a proliferation score of a control sample, wherein the determining the proliferation score comprises: (i) measuring a nucleic acid expression level of at least five classifier genes from a plurality of classifier genes in the control sample, wherein the plurality of classifier genes consists of only tpx2, dlgap5, hjurp, kif4a, kif2c, plk1, melk, ccnb2, bub1, kif23, ube2c, kif20a, troap, aurkb, rrm2, mybl2, mki67, cdc20, cep55, top2a, birc5, aspm, espl1, kif18b, iqgap3 and epr1; and (ii) calculating a mean nucleic acid expression level across the at least five classifier biomarkers from the plurality of classifier biomarkers, wherein the mean nucleic acid expression level across the at least five classifier biomarkers from the plurality of classifier biomarkers represents the proliferation score; and (c) comparing the proliferation score of the sample obtained from the subject to the proliferation score of the control sample, wherein an elevated proliferation score in the sample obtained from the subject is indicative of a poor disease outcome for the subject, wherein the subject suffers from or is suspected of suffering from a cancer selected from LUAD, LGG, LIHC, KIRC, KICH, MESO, ACC and KIRP. In some cases, the disease outcome is expressed as overall patient survival. In some cases, said disease outcome is expressed as recurrence-free survival. In some cases, said disease outcome is expressed as distant recurrence-free survival. In some cases, the control sample is from a healthy subject. In some cases, the control sample is a non-proliferative cancer sample. In some cases, the sample obtained from the subject and/or the control sample is a formalin-fixed, paraffin-embedded (FFPE) tissue sample, fresh or a frozen tissue sample, an exosome, wash fluids, cell pellets, or a bodily fluid obtained from the patient. In some cases, the sample obtained from the subject and/or the control is an FFPE tissue sample. In some cases, the sample obtained from the subject and/or the control is a fresh frozen tissue sample. In some cases, the nucleic acid expression level is measured using an amplification, sequencing or hybridization assay. In some cases, the amplification, hybridization and/or sequencing assay comprises performing quantitative real time reverse transcriptase polymerase chain reaction (qRT-PCR), RNA-seq, microarrays, gene chips, nCounter Gene Expression Assay, Serial Analysis of Gene Expression (SAGE), Rapid Analysis of Gene Expression (RAGE), nuclease protection assays, Northern blotting, nCounter DX Analysis System or any other equivalent gene expression detection techniques. In some cases, the nucleic acid expression level is detected by performing RNA-seq. In some cases, the measuring the nucleic acid expression level is for at least 10, 15, 20 or 25 classifier genes from the plurality of classifier genes. In some cases, the measuring the nucleic acid expression level is for all of the classifier genes from the plurality of classifier genes. In some cases, the method further comprises determining a level and/or activity of at least one additional marker involved in cell proliferation and mitosis. In some cases, the at least one additional marker is Ki67 or CD31. In some cases, the method further comprises determining a subtype of the sample obtained from the subject. In some cases, the determining the subtype is performed by histological examination of the sample. In some cases, the determining the subtype is performed by gene expression analysis of the sample. In some cases, the gene expression analysis of the sample is performed using a gene expression sub-typer that is publicly available.

In one aspect, provided herein is a system for determining an antifolate predictive response signature of a sample obtained from a subject suffering from cancer, the system comprising: (a) one or more processors; and (b) one or more memories operatively coupled to at least one of the one or more processors and having instructions stored thereon that, when executed by at least one of the one or more processors, cause the system to (i) detect an expression level of each of a plurality of classifier biomarkers from Table 1; (ii) compare the expression levels of each of the plurality of classifier biomarkers from Table 1 to the expression levels of each of the plurality of classifier biomarkers from Table 1 in a control; and (iii) classifying the sample as TRU, PP, or PI based on the results of the comparing step. In some cases, the control comprises at least one sample training set(s), wherein the at least one sample training set comprises expression data of the plurality of classifier biomarkers of Table 1 from a reference adenocarcinoma TRU (bronchioid) sample, expression data of the plurality of classifier biomarkers of Table 1 from a reference adenocarcinoma PP (magnoid) sample, expression data of the plurality of classifier biomarkers of Table 1 from a reference adenocarcinoma PI (squamoid) sample, or a combination thereof. In some cases, the comparing step comprises applying a statistical algorithm which comprises determining a correlation between the expression data obtained from the sample and the expression data from the at least one training set(s); and classifying the sample as a TRU, PP, or PI subtype based on the results of the statistical algorithm. In some cases, the expression level of each of the plurality of classifier biomarkers from Table 1 is detected at the nucleic acid level. In some cases, the nucleic acid level is RNA or cDNA. In some cases, the detecting the expression level comprises performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays, Northern blotting, or any other equivalent gene expression detection techniques. In some cases, the expression level is detected by performing qRT-PCR. In some cases, the detecting the expression level is performed using a device that is part of the system or in communication with at least one of the one or more processors, wherein upon receipt of instructions sent by the at least one of the one or more processors, perform the detection of the expression levels. In some cases, the plurality of classifier biomarkers from Table 1 comprises at least 8 classifier biomarkers, at least 16 classifier biomarkers, at least 24 classifier biomarkers, at least 32 classifier biomarkers, at least 40 classifier biomarkers or at least 48 classifier biomarkers from Table 1. In some cases, the plurality of classifier biomarkers of Table 1 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the classifier biomarkers from Table 1. In some cases, the plurality of classifier biomarkers of Table 1 comprise flgf, ctsh, sctr, cyp4b1, gpr116, adh1b, cbx7, hlf cep55, tpx2, bub1b, kif4a, ccnb2, kif14, melk, kif11 or any combination thereof. In some cases, the plurality of classifier biomarkers of Table 1 comprise fgl1, pbk, hspd1, tdg, prc1, dusp4, gtpbp4, zwint, tlr2, cd74, hla-dpb1, hla-dpa1, hla-dra, itgb2, fas, hla-drb1, plau, gbp1, dse, ccdc109b, tgfbi, cxcl10, lgals1, tubb6, gjb1, rap1gap, cacna2d2, selenbp1, tfcp2l1, sorbs2, unc13b, tacc2 or any combination thereof. In some cases, the plurality of classifier biomarkers of Table 1 comprises all the classifier biomarkers from Table 1. In some cases, the TRU subtype is indicative of a positive antifolate predictive response signature, wherein the positive antifolate predictive response signature selects the patient for treatment with an antifolate agent. In some cases, the anti-folate agent is selected from pemetrexed, methotrexate, trimetrexate, lometrexol, raltitrexed and nolatrexed. In some cases, the antifolate agent is pemetrexed. In some cases, the antifolate agent is raltitrexed. In some cases, the cancer the patient is suffering from is selected from bladder cancer, breast cancer, pancreatic adenocarcinoma, lung adenocarcinoma, lung squamous cell carcinoma, and head and neck adenocarcinoma.

In another aspect, provided herein is a system for determining a disease outcome in a subject suffering from or suspected of suffering from cancer, the system comprising: (a) one or more processors; and (b) one or more memories operatively coupled to at least one of the one or more processors and having instructions stored thereon that, when executed by at least one of the one or more processors, cause the system to (i) determine a proliferation score of a sample obtained from the subject, wherein the determining the proliferation score comprises: (a) measuring a nucleic acid expression level of at least five classifier genes from a plurality of classifier genes in the sample obtained from the cancer patient, wherein the plurality of classifier genes consists of only tpx2, dlgap5, hjurp, kif4a, kif2c, plk1, melk, ccnb2, bub1, kif23, ube2c, kif20a, troap, aurkb, rrm2, mybl2, mki67, cdc20, cep55, top2a, birc5, aspm, espl1, kif18b, iqgap3 and epr1; and (b) calculating a mean nucleic acid expression level across the at least five classifier biomarkers from the plurality of classifier biomarkers, wherein the mean nucleic acid expression level across the at least five classifier biomarkers from the plurality of classifier biomarkers represents the proliferation score; (ii) determine a proliferation score of a control sample, wherein the determining the proliferation score comprises: (a) measuring a nucleic acid expression level of at least five classifier genes from a plurality of classifier genes in the control sample, wherein the plurality of classifier genes consists of only tpx2, dlgap5, hjurp, kif4a, kif2c, plk1, melk, ccnb2, bub1, kif23, ube2c, kif20a, troap, aurkb, rrm2, mybl2, mki67, cdc20, cep55, top2a, birc5, aspm, espl1, kif18b, iqgap3 and epr1; and (b) calculating a mean nucleic acid expression level across the at least five classifier biomarkers from the plurality of classifier biomarkers, wherein the mean nucleic acid expression level across the at least five classifier biomarkers from the plurality of classifier biomarkers represents the proliferation score; and (iii) compare the proliferation score of the sample obtained from the subject to the proliferation score of the control sample, wherein an elevated proliferation score in the sample obtained from the subject is indicative of a poor disease outcome for the subject, wherein the subject suffers from or is suspected of suffering from a cancer. In some cases, the cancer is selected from LUAD, LGG, LIHC, KIRC, KICH, MESO, ACC and KIRP. In some cases, the disease outcome is expressed as overall patient survival. In some cases, said disease outcome is expressed as recurrence-free survival. In some cases, said disease outcome is expressed as distant recurrence-free survival. In some cases, the control sample is from a healthy subject. In some cases, the control sample is a non-proliferative cancer sample. In some cases, the sample obtained from the subject and/or the control sample is a formalin-fixed, paraffin-embedded (FFPE) tissue sample, fresh or a frozen tissue sample, an exosome, wash fluids, cell pellets, or a bodily fluid obtained from the patient. In some cases, the sample obtained from the subject and/or the control is an FFPE tissue sample. In some cases, the sample obtained from the subject and/or the control is a fresh frozen tissue sample. In some cases, the nucleic acid expression level is measured using an amplification, sequencing or hybridization assay. In some cases, the amplification, hybridization and/or sequencing assay comprises performing quantitative real time reverse transcriptase polymerase chain reaction (qRT-PCR), RNA-seq, microarrays, gene chips, nCounter Gene Expression Assay, Serial Analysis of Gene Expression (SAGE), Rapid Analysis of Gene Expression (RAGE), nuclease protection assays, Northern blotting, nCounter DX Analysis System or any other equivalent gene expression detection techniques. In some cases, the nucleic acid expression level is detected by performing RNA-seq. In some cases, the detecting the expression level is performed using a device that is part of the system or in communication with at least one of the one or more processors, wherein upon receipt of instructions sent by the at least one of the one or more processors, perform the detection of the expression levels. In some cases, the measuring the nucleic acid expression level is for at least 10, 15, 20 or 25 classifier genes from the plurality of classifier genes. In some cases, the measuring the nucleic acid expression level is for all of the classifier genes from the plurality of classifier genes. In some cases, the method further comprises determining a level and/or activity of at least one additional marker involved in cell proliferation and mitosis. In some cases, the at least one additional marker is Ki67 or CD31.

### BRIEF DESCRIPTION OF THE DRAWINGS

FIG. 1 illustrates a plot of the mean expression value vs. variance of log 2 transformed gene expression values across 30 tumor types from the Cancer Genome Atlas (TCGA) Pan Cancer Atlas data set. As shown by the dotted lines, genes with mean variance and mean expression values greater than 4 (i.e., 2175 genes) were keep and used to develop the proliferation signature described herein.

FIG. 2 illustrates agglomerative hierarchical clustering with average linkage and correlation for distance of the 2175 gene selected from TCGA Pan Cancer dataset. A sub-cluster of 26 genes was identified in the upper, right corner of the resulting clustering dendrogram that showed high gene-gene correlation coefficients and were selected as a proliferation signature (see Table 2).

FIGS. 3A and 3B illustrates comparisons of the proliferation signature (Table 2) described herein with the PAM50 proliferation signature described in Nielsen, Torsten O., Joel S. Parker, Samuel Leung, David Voduc, Mark Ebbert, Tammi Vickery, Sherri R. Davies et al. “A comparison of PAM50 intrinsic subtyping with immunohistochemistry and clinical prognostic factors in tamoxifen-treated estrogen receptor-positive breast cancer.” Clinical cancer research (2010): 1078-0432 in both a training data set (FIG. 3A) used to generate the Table 2 proliferation signature and a test data set (FIG. 3B). Both the training and testing data sets were derived from TCGA Pan Cancer dataset and were balanced for uniform tumor type distributions across 30 tumor types. The 30 tumor types included kidney renal papillary cell carcinoma (KIRP); breast invasive carcinoma (BRCA); thyroid cancer (THCA); bladder urothelial carcinoma (BLCA); prostate adenocarcinoma (PRAD); kidney chromophobe (KICH); cervical squamous cell carcinoma and endocervical adenocarcinoma (CESC); kidney renal clear cell carcinoma (KIRC); liver hepatocellular carcinoma (LIHC); low grade glioma (LGG); sarcoma (SARC); lung adenocarcinoma (LUAD); colon adenocarcinoma (COAD); head and neck squamous cell carcinoma (HNSC); uterine corpus endometrial carcinoma (UCEC); glioblastoma multiforme (GBM); esophageal carcinoma (ESCA); stomach adenocarcinoma (STAD); ovarian serous cystadenocarcinoma (OV); rectum adenocarcinoma (READ); adrenocortical carcinoma (ACC); uveal melanoma (UVM); mesothelioma (MESO); pheochromocytoma and paraganglioma (PCPG); skin cutaneous melanoma (SKCM); uterine carcinsarcoma (UCS); lung squamous cell carcinoma (LUSC); testicular germ cell tumors (TGCT); cholangiocarcinoma (CHOL); pancreatic adenocarcinoma (PAAD).

FIG. 4 shows a table containing within-tumor type survival-proliferation cox model hazard ratios (HR) and p-values (p) resulting from an analysis of the association between overall survival and the Table 2 proliferation signature using the test data set.

FIG. 5A shows boxplots of the association between proliferation score (Y-axis) and intrinsic gene expression based multiple myeloma (MM) subtypes I-VII. Proliferation score was determined for each sample using the Table 2 proliferation signature, while subtyping was done using the expression data from Chapman M A, et al. (2011) “Initial genome sequencing and analysis of multiple myeloma.” Nature 2011 Mar. 24; 471(7339):467-72. FIG. 5B shows a Kaplan-Meier plot of the association between proliferation and disease-specific survival (i.e., multiple myeloma) where patients have been grouped by proliferation quartiles.

FIG. 6 shows boxplots of the association between pemetrexed drug targets (i.e., DHFR, GART, TYMS, ATIC and MTHFD1L) and proliferation score and intrinsic gene expression-based lung adenocarcinoma (LUAD) subtypes (i.e., bronchioid, magnoid, squamoid) from TCGA LUAD dataset (n=515). Proliferation score was determined for each sample using the Table 2 proliferation signature, while subtyping was done using the 48-gene LUAD subtyper found in Table 1 of WO 2017/201165, which is herein incorporated by reference and recreated as Table 4 herein.

FIG. 7 shows boxplots of the association between pemetrexed drug target TYMS, proliferation score and antifolate predictive response signature (AF-PRS) positive (+) (i.e., bronchioid, LUAD subtype) and AF-PRS negative (−) (i.e., magnoid and squamoid LUAD subtypes) from TCGA LUAD dataset (n=515). Proliferation score was determined for each sample using the Table 2 proliferation signature, while AF-PRS subtyping was done using the 48-gene LUAD subtyper found in Table 1 of WO 2017/201165, which is herein incorporated by reference and recreated as Table 4 herein.

FIG. 8 shows boxplots of the association between tumor mutational burden (TMB) and antifolate predictive response signature (AF-PRS) positive (+) (i.e., bronchioid, LUAD subtype) and AF-PRS negative (−) (i.e., magnoid and squamoid LUAD subtypes).

FIG. 9 shows boxplots of the association between pemetrexed drug targets (i.e., DHFR, GART, TYMS, ATIC and MTHFD1L) and proliferation score and intrinsic gene expression-based bladder cancer (BLCA) subtypes from TCGA BLCA dataset (n=408).

FIG. 10 shows boxplots of the association between pemetrexed drug targets (i.e., DHFR, GART, TYMS, ATIC and MTHFD1L) and proliferation score and intrinsic gene expression-based breast cancer (BRCA) subtypes from TCGA BRCA dataset (n=513).

FIG. 11 shows boxplots of the association between pemetrexed drug targets (i.e., DHFR, GART, TYMS, ATIC and MTHFD1L) and proliferation score and intrinsic gene expression-based head and neck squamous cell carcinoma (HNSCC) subtypes from TCGA HNSCC dataset (n=520).

FIG. 12 shows boxplots of the association between pemetrexed drug targets (i.e., DHFR, GART, TYMS, ATIC and MTHFD1L) and proliferation score and intrinsic gene expression based pancreatic adenocarcinoma (PAAD) subtypes from TCGA PAAD dataset (n=150).

FIG. 13 shows boxplots of the association between pemetrexed drug targets (i.e., DHFR, GART, TYMS, ATIC and MTHFD1L) and proliferation score and intrinsic gene expression-based lung adenocarcinoma (LUAD) subtypes (i.e., bronchioid, magnoid, squamoid) in BLCA from TCGA BLCA dataset (n=408). Subtyping was done using the 48-gene LUAD subtyper found in Table 1 of WO 2017/201165, which is herein incorporated by reference and recreated as Table 4 herein.

FIG. 14 shows boxplots of the association between pemetrexed drug targets (i.e., DHFR, GART, TYMS, ATIC and MTHFD1L) and proliferation score and intrinsic gene expression-based lung adenocarcinoma (LUAD) subtypes (i.e., bronchioid, magnoid, squamoid) in BRCA from TCGA BRCA dataset (n=513). Subtyping was done using the 48-gene LUAD subtyper found in Table 1 of WO 2017/201165, which is herein incorporated by reference and recreated as Table 4 herein.

FIG. 15 shows boxplots of the association between pemetrexed drug targets (i.e., DHFR, GART, TYMS, ATIC and MTHFD1L) and proliferation score and intrinsic gene expression-based lung adenocarcinoma (LUAD) subtypes (i.e., bronchioid, magnoid, squamoid) in HNSCC from TCGA HNSCC dataset (n=520). Subtyping was done using the 48-gene LUAD subtyper found in Table 1 of WO 2017/201165, which is herein incorporated by reference and recreated as Table 4 herein.

FIG. 16 shows boxplots of the association between pemetrexed drug targets (i.e., DHFR, GART, TYMS, ATIC and MTHFD1L) and proliferation score and intrinsic gene expression-based lung adenocarcinoma (LUAD) subtypes (i.e., bronchioid, magnoid, squamoid) in PAAD from TCGA PAAD dataset (n=150). Subtyping was done using the 48-gene LUAD subtyper found in Table 1 of WO 2017/201165, which is herein incorporated by reference and recreated as Table 4 herein.

FIG. 17 illustrates overall survival (OS) probability by anti-folate predictive response signature sign (i.e., AF-PRS (+) or AF-PRS(−)) as determined for the TCGA non-small cell lung cancer (NSCLC) lung adenocarcinoma (LUAD) dataset containing overall survival data (n=506) using stratified cox models and Kaplan Meier plots.

FIG. 18 shows boxplots of the association between pemetrexed drug targets (i.e., DHFR, GART, TYMS, ATIC and MTHFD1L) and proliferation score and antifolate predictive response signature (AF-PRS) positive (+) (i.e., bronchioid, LUAD subtype) and AF-PRS negative (−) (i.e., magnoid and squamoid LUAD subtypes) in BLCA from TCGA BLCA dataset (n=408). Subtyping was done using the 48-gene LUAD subtyper found in Table 1 of WO 2017/201165, which is herein incorporated by reference and recreated as Table 4 herein.

FIG. 19 shows boxplots of the association between pemetrexed drug targets (i.e., DHFR, GART, TYMS, ATIC and MTHFD1L) and proliferation score and antifolate predictive response signature (AF-PRS) positive (+) (i.e., bronchioid, LUAD subtype) and AF-PRS negative (−) (i.e., magnoid and squamoid LUAD subtypes) in BRCA from TCGA BRCA dataset (n=513). Subtyping was done using the 48-gene LUAD subtyper found in Table 1 of WO 2017/201165, which is herein incorporated by reference and recreated as Table 4 herein.

FIG. 20 shows boxplots of the association between pemetrexed drug targets (i.e., DHFR, GART, TYMS, ATIC and MTHFD1L) and proliferation score and antifolate predictive response signature (AF-PRS) positive (+) (i.e., bronchioid, LUAD subtype) and AF-PRS negative (−) (i.e., magnoid and squamoid LUAD subtypes) in HNSCC from TCGA HNSCC dataset (n=520). Subtyping was done using the 48-gene LUAD subtyper found in Table 1 of WO 2017/201165, which is herein incorporated by reference and recreated as Table 4 herein.

FIG. 21 shows boxplots of the association between pemetrexed drug targets (i.e., DHFR, GART, TYMS, ATIC and MTHFD1L) and proliferation score and antifolate predictive response signature (AF-PRS) positive (+) (i.e., bronchioid, LUAD subtype) and AF-PRS negative (−) (i.e., magnoid and squamoid LUAD subtypes) in LUSC from TCGA LUSC dataset (n=505). Subtyping was done using the 48-gene LUAD subtyper found in Table 1 of WO 2017/201165, which is herein incorporated by reference and recreated as Table 4 herein.

FIG. 22A-B shows expression of genes associated with antifolate (Pemetrexed) activity by LUAD Subtype (FIG. 22A) or AF-PRS Status (FIG. 22B)—Overall Piedmont Study (n=240).

FIG. 23A-B shows progression-free survival probability by AF-PRS Status (FIG. 23B) or lung adenocarcinoma (LUAD) subtype (FIG. 23B) in patients stage I-IV at time of treatment—Piedmont Study Pemetrexed-Platinum Patients (n=95).

FIG. 24 shows progression-free survival probability by AF-PRS Status in patients stage I-III at time of treatment—Piedmont Study Pemetrexed-Platinum Patients (n=26).

FIGS. 25A-25B shows evaluation of complete responses in patients Stage I-IV at the time of treatment (FIG. 25A) and representative scans from Stage IV patients who were AF-PRS(+) (FIG. 25B)—Piedmont Study Pemetrexed-Platinum Patients (n=95).

FIG. 26 shows a schematic of genes associated with antifolate (Pemetrexed) activity as well as cellular influx/efflux and their expression in either the cohort of patients examined in the Piedmont Study (see Example 7; n=95) or the TCGA LUAD cohort (n=515).

### DETAILED DESCRIPTION

### Definitions

While the following terms are believed to be well understood by one of ordinary skill in the art, the following definitions are set forth to facilitate explanation of the presently disclosed subject matter.

As used herein, the singular forms “a”, “an” and “the” are intended to include the plural forms as well, unless the context clearly indicates otherwise. Additionally, the use of “or” is intended to include “and/or” unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The term “about” as used herein can refer to a range that is 15%, 10%, 8%, 6%, 4%, or 2% plus or minus from a stated numerical value.

Unless the context requires otherwise, throughout the present specification and claims, the word “comprise” and variations thereof, such as, “comprises” and “comprising” are to be construed in an open, inclusive sense that is as “including, but not limited to”. The use of the alternative (e.g., “or”) should be understood to mean either one, both, or any combination thereof of the alternatives. As used herein, the terms “about” and “consisting essentially of” mean+/−20% of the indicated range, value, or structure, unless otherwise indicated.

Reference throughout this specification to “one embodiment” or “an embodiment” means that a particular feature, structure or characteristic described in connection with the embodiment may be included in at least one embodiment of the present disclosure. Thus, the appearances of the phrases “in one embodiment” or “in an embodiment” in various places throughout this specification may not necessarily all be referring to the same embodiment. It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination.

Throughout this disclosure, various aspects of the methods and compositions provided herein can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Unless otherwise indicated, the methods and compositions provided herein can utilize conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Gait, “Oligonucleotide Synthesis: A Practical Approach” 1984, IRL Press, London, Nelson and Cox (2000), Lehninger et al., (2008) Principles of Biochemistry 5th Ed., W.H. Freeman Pub., New York, N.Y. and Berg et al. (2006) Biochemistry, 6.sup.th Ed., W.H. Freeman Pub., New York, N.Y., all of which are herein incorporated in their entirety by reference for all purposes.

Conventional software and systems may also be used in the methods and compositions provided herein. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes, etc. The computer-executable instructions may be written in a suitable computer language or combination of several languages. Basic computational biology methods are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2.sup.nd ed., 2001). See U.S. Pat. No. 6,420,108.

The methods and compositions provided herein may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170. Computer methods related to genotyping using high density microarray analysis may also be used in the present methods, see, for example, US Patent Pub. Nos. 20050250151, 20050244883, 20050108197, 20050079536 and 20050042654.

Additionally, the present disclosure may have preferred embodiments that include methods for providing genetic information over networks such as the Internet as shown in U.S. Patent Pub. Nos. 20030097222, 20020183936, 20030100995, 20030120432, 20040002818, 20040126840, and 20040049354.

As used herein, the term “individual”, “patient”, or “subject”, can be used interchangeably and can refer to an individual regardless of health and/or disease status. A subject can be a subject, a study participant, a control subject, a screening subject, or any other class of individual from whom a sample can be obtained and assessed in the context of the invention. Accordingly, a subject can be diagnosed with a cancer (including subtypes, or grades thereof), can present with one or more symptoms of a cancer or a predisposing factor, such as a family (genetic) or medical history (medical) factor, for a cancer, can be undergoing treatment or therapy for a cancer, or the like. Alternatively, a subject can be healthy with respect to any of the aforementioned factors or criteria.

It will be appreciated that the term “healthy” as used herein, can be relative to a cancer status, as the term “healthy” cannot be defined to correspond to any absolute evaluation or status. Thus, an individual defined as healthy with reference to any specified disease or disease criterion, can in fact be diagnosed with any other one or more diseases, or exhibit any other one or more disease criterion, including one or more other cancer types.

As used herein, the terms “individual,” “patient,” and “subject” can refer to any single animal, more preferably a mammal (including such non-human animals as, for example, dogs, cats, horses, rabbits, zoo animals, cows, pigs, sheep, and non-human primates) for which treatment is desired. In particular embodiments, the individual or patient herein is a human.

Further to any of the embodiments provided herein, the cancer can include, but are not limited to, carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. Examples of a cancer also include, but are not limited to, a lung cancer (e.g., a non-small cell lung cancer (NSCLC)), a kidney cancer (e.g., a kidney urothelial carcinoma or RCC), a bladder cancer (e.g., a bladder urothelial (transitional cell) carcinoma (e.g., locally advanced or metastatic urothelial cancer, including 1L or 2L+ locally advanced or metastatic urothelial carcinoma), a breast cancer, a colorectal cancer (e.g., a colon adenocarcinoma), an ovarian cancer, a pancreatic cancer (e.g., pancreatic adenocarcinoma or PAAD), a gastric carcinoma, an esophageal cancer, a mesothelioma, a melanoma (e.g., a skin melanoma), a head and neck cancer (e.g., a head and neck squamous cell carcinoma (HNSCC)), a thyroid cancer, a sarcoma (e.g., a soft-tissue sarcoma, a fibrosarcoma, a myxosarcoma, a liposarcoma, an osteogenic sarcoma, an osteosarcoma, a chondrosarcoma, an angiosarcoma, an endotheliosarcoma, a lymphangiosarcoma, a lymphangioendotheliosarcoma, a leiomyosarcoma, or a rhabdomyosarcoma), a prostate cancer, a glioblastoma, a cervical cancer, a thymic carcinoma, a leukemia (e.g., an acute lymphocytic leukemia (ALL), an acute myelocytic leukemia (AML), a chronic myelocytic leukemia (CML), a chronic eosinophilic leukemia, or a chronic lymphocytic leukemia (CLL)), a lymphoma (e.g., a Hodgkin lymphoma or a non-Hodgkin lymphoma (NHL)), a myeloma (e.g., a multiple myeloma (MM)), a mycosis fungoides, a Merkel cell cancer, a hematologic malignancy, a cancer of hematological tissues, a B cell cancer, a bronchus cancer, a stomach cancer, a brain or central nervous system cancer, a peripheral nervous system cancer, a uterine or endometrial cancer, a cancer of the oral cavity or pharynx, a liver cancer, a testicular cancer, a biliary tract cancer, a small bowel or appendix cancer, a salivary gland cancer, an adrenal gland cancer, an adenocarcinoma, an inflammatory myofibroblastic tumor, a gastrointestinal stromal tumor (GIST), a colon cancer, a myelodysplastic syndrome (MDS), a myeloproliferative disorder (MPD), a polycythemia Vera, a chordoma, a synovioma, an Ewing's tumor, a squamous cell carcinoma, a basal cell carcinoma, an adenocarcinoma, a sweat gland carcinoma, a sebaceous gland carcinoma, a papillary carcinoma, a papillary adenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilms' tumor, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodendroglioma, a meningioma, a neuroblastoma, a retinoblastoma, a follicular lymphoma, a diffuse large B-cell lymphoma, a mantle cell lymphoma, a hepatocellular carcinoma, a thyroid cancer, a small cell cancer, an essential thrombocythemia, an agnogenic myeloid metaplasia, a hypereosinophilic syndrome, a systemic mastocytosis, a familiar hypereosinophilia, a neuroendocrine cancer, or a carcinoid tumor.

In some cases, the cancer is selected from a cervical kidney renal papillary cell carcinoma (KIRP); breast invasive carcinoma (BRCA); thyroid cancer (THCA); bladder carcinoma (BLCA); prostate adenocarcinoma (PRAD); kidney chromophobe (KICH); cervical squamous cell carcinoma and endocervical adenocarcinoma (CESC); kidney renal clear cell carcinoma (KIRC); liver hepatocellular carcinoma (LIHC); low grade glioma (LGG); sarcoma (SARC); lung adenocarcinoma (LUAD); colon adenocarcinoma (COAD); head-neck squamous cell carcinoma. (HNSC); uterine corpus endometrial carcinoma (UCEC); glioblastona multiforme (GBM); esophageal carcinoma (ESCA); stomach adenocarcinoma (STAD): ovarian cancer (OV), rectum adenocarcinoma (READ) or lung squamous cell carcinoma (LUSC), an esophageal cancer, a mesothelioma, a melanoma, a head and neck cancer, a thyroid cancer, a sarcoma, a prostate cancer, a glioblastoma, a cervical cancer, a thymic carcinoma, a leukemia, a lymphoma, a myeloma, a mycosis fungoides, a merkel cell cancer, an endometrial cancer. In some cases, the cancer is lung adenocarcinoma (LUAD); colon adenocarcinoma (COAD), breast invasive carcinoma (BRCA), uterine corpus endometrial carcinoma (UCEC), rectum adenocarcinoma (READ) or lung squamous cell carcinoma (LUSC).

The term “nucleic acid” as used herein can refer to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus, the terms nucleoside, nucleotide, deoxynucleoside and deoxynucleotide generally include analogs such as those described herein. These analogs can be those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs can be derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

The term “complementary” as used herein can refer to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. See, M. Kanehisa Nucleic Acids Res. 12:203 (1984), incorporated herein by reference.

An analyte assay can be a detection or diagnostic method as provided herein. In some cases, the sample can comprise or contain the analyte. The analyte can be cell-free or extracellular nucleic acid. In some cases, the analyte is a circulating tumor nucleic acid. The nucleic acid can be such DNA or RNA. In some cases, the nucleic acid is cell-free DNA (cfDNA). The cfDNA can be circulating tumor DNA (ctDNA). The sample can be a biological sample, such as a liquid biological sample or bodily fluid or a biological tissue. Examples of liquid biological samples or bodily fluids for use in the methods provided herein can include urine, blood, plasma, serum, saliva, ejaculate, stool, sputum, cerebrospinal fluid (CSF), tears, mucus, amniotic fluid or the like. Biological tissues are aggregates of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s). A biological tissue sample can be a biopsy. In one embodiment, the sample is a biopsy of a tumor, which can be referred to as a tumor sample. In one embodiment, the analyses described herein are performed on biopsies that are embedded in paraffin wax. Accordingly, the methods provided herein, including the RT-PCR methods, are sensitive, precise and have multianalyte capability for use with paraffin embedded samples. See, for example, Cronin et al. (2004) Am. J Pathol. 164(1):35-42, herein incorporated by reference.

Formalin fixation and tissue embedding in paraffin wax is a universal approach for tissue processing prior to light microscopic evaluation. A major advantage afforded by formalin-fixed paraffin-embedded (FFPE) specimens is the preservation of cellular and architectural morphologic detail in tissue sections. (Fox et al. (1985) J Histochem Cytochem 33:845-853). The standard buffered formalin fixative in which biopsy specimens are processed is typically an aqueous solution containing 37% formaldehyde and 10-15% methyl alcohol. Formaldehyde is a highly reactive dipolar compound that results in the formation of protein-nucleic acid and protein-protein crosslinks in vitro (Clark et al. (1986) J Histochem Cytochem 34:1509-1512; McGhee and von Hippel (1975) Biochemistry 14:1281-1296, each incorporated by reference herein).

In one embodiment, the sample used herein is obtained from an individual, and comprises fresh-frozen paraffin embedded (FFPE) tissue.

The term “tumor,” as used herein, can refer to all neoplastic cell growth and proliferation, whether malignant or benign, and all pre-cancerous and cancerous cells and tissues. The terms “cancer,” “cancerous,” and “tumor” are not mutually exclusive and can be used interchangeably.

The term “detection” can include any means of detecting, including direct and indirect detection.

The sample can be processed to render it competent for fragmentation, ligation, denaturation, and/or amplification. Exemplary sample processing can include lysing cells of the sample to release nucleic acid, purifying the sample (e.g., to isolate nucleic acid from other sample components, which can inhibit enzymatic reactions), diluting/concentrating the sample, and/or combining the sample with reagents for further nucleic acid processing such as nucleic acid extension, amplification and/or sequencing. In some examples, the sample can be combined with a restriction enzyme, reverse transcriptase, or any other enzyme of nucleic acid processing.

The term “biomarkers” or “classifier biomarkers” or “classifier” can include nucleic acids (e.g., genes) and proteins, and variants and fragments thereof. Such biomarkers can include RNA or DNA, including cDNA, comprising the entire or partial sequence of the nucleic acid sequence encoding the biomarker, or the complement of such a sequence. The biomarker nucleic acids can also include any expression product or portion thereof of the nucleic acid sequences of interest. A biomarker protein is a protein encoded by or corresponding to a DNA or RNA biomarker of the invention. A biomarker protein comprises the entire or partial amino acid sequence of any of the biomarker proteins or polypeptides. The biomarker nucleic acid can be extracted from a cell or can be cell free or extracted from an extracellular vesicular entity such as an exosome or microvesicles.

A “biomarker” or “classifier biomarker” or “classifier” can be any nucleic acid (e.g., gene) or protein whose level of expression in a tissue or cell is altered compared to that of a normal or healthy cell or tissue. The detection, and in some cases the level, of the biomarkers can permit the differentiation of samples. The “classifier biomarker” or “biomarker” or “classifier” may be one that is up-regulated (e.g., expression is increased) or down-regulated (e.g., expression is decreased) relative to a reference or control as provided herein. The overall expression level in each gene cassette is referred to herein as the “expression profile” and is used to classify a test sample. However, it is understood that independent evaluation of expression for each of the genes disclosed herein can be used to classify a test sample (e.g., as being an antifolate responsive group or not and/or possessing tumor proliferation) without the need to group up-regulated and down-regulated genes into one or more gene cassettes. In some cases, as shown in Table 1, a total of 48 biomarkers or a subset of the 48 biomarkers of Table 1 can be used for assessment of an antifolate predictive response. In some cases, as shown in Table 2, a total of 26 biomarkers or a subset of the 26 biomarkers of Table 2 can be used for assessment of proliferation.

As used herein, an “expression profile” or a “biomarker profile” or “gene signature” comprises one or more values corresponding to a measurement of the relative abundance, level, presence, or absence of expression of a discriminative or classifier gene or biomarker. An expression profile can be derived from a subject prior to or subsequent to a diagnosis of a cancer, can be derived from a biological sample collected from a subject at one or more time points prior to or following treatment or therapy, can be derived from a biological sample collected from a subject at one or more time points during which there is no treatment or therapy, or can be collected from a healthy subject. The term subject can be used interchangeably with patient. The patient can be a human patient. The one or more biomarkers of the biomarker profiles provided herein are selected from one or more biomarkers of Table 1 or Table 2.

As used herein, the term “determining an expression level” or “determining an expression profile” or “detecting an expression level” or “detecting an expression profile” as used in reference to a biomarker or classifier means the application of a biomarker specific reagent such as a probe, primer or antibody and/or a method to a sample, for example a sample of the subject or patient and/or a control sample, for ascertaining or measuring quantitatively, semi-quantitatively or qualitatively the amount of a biomarker or biomarkers, for example the amount of biomarker polypeptide or mRNA (or cDNA derived therefrom). For example, a level of a biomarker can be determined by a number of methods including for example immunoassays including for example immunohistochemistry, ELISA, Western blot, immunoprecipitation and the like, where a biomarker detection agent such as an antibody for example, a labeled antibody, specifically binds the biomarker and permits for example relative or absolute ascertaining of the amount of polypeptide biomarker, hybridization and PCR protocols where a probe or primer or primer set are used to ascertain the amount of nucleic acid biomarker, including for example probe based and amplification based methods including for example microarray analysis, RT-PCR such as quantitative RT-PCR (qRT-PCR), serial analysis of gene expression (SAGE), Northern Blot, digital molecular barcoding technology, for example Nanostring Counter Analysis, and TaqMan quantitative PCR assays. Other methods of mRNA detection and quantification can be applied, such as mRNA in situ hybridization in formalin-fixed, paraffin-embedded (FFPE) tissue samples or cells. This technology is currently offered by the QuantiGene ViewRNA (Affymetrix), which uses probe sets for each mRNA that bind specifically to an amplification system to amplify the hybridization signals; these amplified signals can be visualized using a standard fluorescence microscope or imaging system. This system for example can detect and measure transcript levels in heterogeneous samples; for example, if a sample has normal and tumor cells present in the same tissue section. As mentioned, TaqMan probe-based gene expression analysis (PCR-based) can also be used for measuring gene expression levels in tissue samples, and this technology has been shown to be useful for measuring mRNA levels in FFPE samples. In brief, TaqMan probe-based assays utilize a probe that hybridizes specifically to the mRNA target. This probe contains a quencher dye and a reporter dye (fluorescent molecule) attached to each end, and fluorescence is emitted only when specific hybridization to the mRNA target occurs. During the amplification step, the exonuclease activity of the polymerase enzyme causes the quencher and the reporter dyes to be detached from the probe, and fluorescence emission can occur. This fluorescence emission is recorded, and signals are measured by a detection system; these signal intensities are used to calculate the abundance of a given transcript (gene expression) in a sample.

The present invention also encompasses a system capable of distinguishing various subtypes of cancer that may or may not be amendable to treatment with an antifolate agent and/or assessing levels of proliferation in a sample obtained from a subject suspected of suffering from cancer. This system can be capable of processing a large number of subjects and subject variables such as expression profiles and other diagnostic criteria. The methods and systems incorporating said methods described herein can be used for “pharmacometabonomics,” in analogy to pharmacogenomics, e.g., predictive of response to therapy. In this embodiment, subjects could be divided into “responders” and “nonresponders” using the expression profile and/or level of proliferation or proliferation score as evidence of “response,” and features of the expression profile and/or level of proliferation or proliferation score could then be used to target future subjects who would likely respond to a particular therapeutic course.

The expression profile and/or level of proliferation or proliferation score can be used in combination with other diagnostic methods including histochemical, immunohistochemical, cytologic, immunocytologic, and visual diagnostic methods including histologic or morphometric evaluation of lung tissue.

In various embodiments of the present invention, the expression profile or signature derived from a subject is compared to a reference expression profile or signature. A “reference expression profile” can be a profile derived from the subject prior to treatment or therapy; can be a profile produced from the subject sample at a particular time point (usually prior to or following treatment or therapy but can also include a particular time point prior to or following diagnosis of lung cancer); or can be derived from a healthy individual or a pooled reference from healthy individuals. A reference expression profile can be specific to cancer types or subtypes known to be responders to antifolate therapy or non-responders to antifolate therapy. A reference expression profile can be specific to cancer types or subtypes known to be proliferative or non-proliferative.

The reference expression profile or signature can be compared to a test expression profile or signature. A “test expression profile” can be derived from the same subject as the reference expression profile except at a subsequent time point (e.g., one or more days, weeks or months following collection of the reference expression profile) or can be derived from a different subject. In summary, any test expression profile of a subject can be compared to a previously collected profile from a subject whose cancer type or subtype is known to be responsive to antifolate therapy or non-responsive to antifolate therapy and/or proliferative or non-proliferative.

### Overview-Anti-Folate Signature

The present invention provides methods, compositions or kits that can be used to provide assessment or determination of an expression profile of a defined set of biomarkers in a sample obtained from a subject suffering from or suspected of suffering from a cancer such that the expression profile can be predictive of said subject being responsive or non-responsive to a defined set of therapeutic agents. The sample can be any sample provided herein. The cancer can be any cancer provided herein. The therapeutic agents can be drugs that are classified as antifolate agents such as, for example, pemetrexed, methotrexate, trimetrexate, lometrexol, raltitrexed and nolatrexed. In one embodiment, the likelihood of a subject suffering from or suspected of suffering from a cancer being responsive to treatment with an antifolate drug or agent is assessed through the evaluation of expression patterns or profiles of a plurality of classifier genes or biomarkers selected from the classifier genes or biomarkers listed in Table 1. Further to this embodiment, the “expression profile” or a “biomarker profile” or “gene signature” associated with the gene cassettes or classifier genes described in Table 1 can be useful for distinguishing between subjects who may be responsive and subjects who may be non-responsive to treatment with anti-folate agents in any type of cancer. As such, the set of biomarkers listed in Table 1 can be referred to as an anti-folate predictive response signature. Subjects whose profile of expression of a plurality of biomarkers from Table 1 indicate that said subject may be responsive to treatment with an antifolate agent or drug can have a positive antifolate predictive response signature (AF-PRS (+)), while subjects whose profile of expression of a plurality of biomarkers from Table 1 indicates that said subject may not be responsive to treatment with an antifolate agent or drug can have a negative antifolate predictive response signature (AF-PRS (−)). The expression level of any and all genes utilized in an antifolate predictive response signature as provided herein can be normalized as provided herein, such as, for example, normalizing expression of the classifier genes by using expression levels from one or more reference or housekeeping genes.

TABLE 1
Antifolate Predictive Response Signature

| | | | GenBank | SEQ |
|---|---|---|---|---|
| | | | Acession | ID |
| Gene # | Gene Symbol | Gene Name | Number* | NO: |
| 1 | FIGF | C-fos-induced growth factor | AY874421.1 | 1 |
| 2 | CTSH | Cathepsin H | NM_004390.4 | 2 |
| 3 | SCTR | Secretin receptor | NM_002980.2 | 3 |
| 4 | CYP4B1 | Cytochrome P450 family 4 | NM_001319161.1 | 4 |
| | | subfamily B member 1 | | |
| 5 | GPR116 | G protein-coupled receptor | AY140958.1 | 5 |
| | | 116 | | |
| 6 | ADH1B | Alcohol dehydrogenase 1B | NM_001286650.1 | 6 |
| | | (class I) | | |
| 7 | CBX7 | Chromobox 7 | NM_175709.3 | 7 |
| 8 | HLF | Hepatic leukemia factor | M95585.1 | 8 |
| 9 | CEP55 | Centrosomal protein 55 | NM_018131.4 | 9 |
| 10 | TPX2 | Tpx2, Microtubule- | NM_012112.4 | 10 |
| | | associated | | |
| 11 | BUB1B | BUB1 mitotic checkpoint | AF107297.1 | 11 |
| | | serine/threonine kinase B | | |
| 12 | KIF4A | Kinesin family member 4A | NM_012310.4 | 12 |
| 13 | CCNB2 | Cyclin B2 | NM_004701.3 | 13 |
| 14 | KIF14 | Kinesin family member 14 | NM_014875.2 | 14 |
| 15 | MELK | Maternal embryonic leucine | NM_014791.3 | 15 |
| | | zipper kinase | | |
| 16 | KIF11 | Kinesin family member 11 | NM_004523.3 | 16 |
| 17 | FGL1 | Fibrinogen like 1 | NM_004467.3 | 17 |
| 18 | PBK | PDZ binding kinase | NM_018492.3 | 18 |
| 19 | HSPD1 | Heat shock protein family D | NM_002156.4 | 19 |
| | | (Hsp60) member 1 | | |
| 20 | TDG | Thymine DNA glycosylase | NM_003211.4 | 20 |
| 21 | PRC1 | Protein regulator of | NM_003981.3 | 21 |
| | | cytokinesis 1 | | |
| 22 | DUSP4 | Dual specificity phosphatase | NM_001394.6 | 22 |
| | | 4 | | |
| 23 | GTPBP4 | GTP binding protein 4 | NM_012341.2 | 23 |
| 24 | ZWINT | ZW10 interacting | NM_007057.3 | 24 |
| | | kinetochore protein | | |
| 25 | TLR2 | Toll like receptor 2 | NM_001318787.1 | 25 |
| 26 | CD74 | CD74 molecule | NM_001025159.2 | 26 |
| 27 | HLA-DPB1 | Major histocompatibility | M83664.1 | 27 |
| | | complex, class II, DP beta 1 | | |
| 28 | HLA-DPA1 | Major histocompatibility | NM_033554.3 | 28 |
| | | complex, class II, DP alpha 1 | | |
| 29 | HLA-DRA | Major histocompatibility | NM_019111.4 | 29 |
| | | complex, class II, DR alpha | | |
| 30 | ITGB2 | Integrin subunit beta 2 | NM_000211.4 | 30 |
| 31 | FAS | Fas cell surface death | KM114217.1 | 31 |
| | | receptor | | |
| 32 | HLA-DRB1 | Major histocompatibility | NM_002124.3 | 32 |
| | | complex, class II, DR beta 1 | | |
| 33 | PLAU | Plasminogen activator, | NM_002658.4 | 33 |
| | | urokinase | | |
| 34 | GBP1 | Guanylate binding protein 1 | NM_002053.2 | 34 |
| 35 | DSE | Dermatan sulfate epimerase | NM_013352.3 | 35 |
| 36 | CCDC109B | Coiled-coil domain | BC002633.2 | 36 |
| | | containing 109B | | |
| 37 | TGFBI | Transforming growth factor | NM_000358.2 | 37 |
| | | beta induced | | |
| 38 | CXCL10 | C-X-C motif chemokine | NM_001565.3 | 38 |
| | | ligand 10 | | |
| 39 | LGALS1 | Lectin, galactoside binding | NM_002305.3 | 39 |
| | | soluble 1 | | |
| 40 | TUBB6 | Tubulin beta 6 class V | NM_032525.2 | 40 |
| 41 | GJB1 | Gap junction protein beta 1 | NM_001097642.2 | 41 |
| 42 | RAP1GAP | RAP1 GTPase activating | NM_001145658.1 | 42 |
| | | protein | | |
| 43 | CACNA2D2 | Calcium voltage-gated | NM_001005505.2 | 43 |
| | | channel auxiliary subunit | | |
| | | alpha2delta 2 | | |
| 44 | SELENBP1 | Selenium binding protein 1 | NM_003944.3 | 44 |
| 45 | TFCP2L1 | Transcription factor CP2-like | NM_014553.2 | 45 |
| | | 1 | | |
| 46 | SORBS2 | Sorbin and SH3 domain | NM_001270771.1 | 46 |
| | | containing 2 | | |
| 47 | UNC13B | Unc-13 homolog B | NM_006377.3 | 47 |
| 48 | TACC2 | Transforming acidic coiled- | AF220152.2 | 48 |
| | | coil containing protein 2 | | |

*Each GenBank Accession Number is a representative or exemplary GenBank Accession Number for the listed gene and is herein incorporated by reference in its entirety for all purposes. Further, each listed representative or exemplary accession number should not be construed to limit the claims to the specific accession number.

In one embodiment, determining or detecting the expression of a plurality of biomarkers from the anti-folate predictive response signature of Table 1 in a sample (e.g., tumor sample) obtained from a subject suffering from or suspected of suffering from a cancer is used to determine if a subtype of the sample is akin or similar to a bronchioid (i.e., Terminal Respiratory Unit), or non-bronchioid (i.e., squamoid (Proximal Inflammatory) or magnoid (Proximal Proliferative)) subtype of lung adenocarcinoma (LUAD). Further to this embodiment, an expression profile of a plurality of biomarkers selected from Table 1 in a sample obtained from a subject suffering from a cancer can be used to determine whether or not the subtype of the subject's cancer can be classified as being a bronchioid or non-bronchioid subtype of LUAD regardless of the type of cancer. The cancer does not have to be lung cancer or LUAD specifically. The cancer can be any cancer known in the art and/or provided herein. In some cases, the cancer is bladder cancer (BLCA), breast cancer (BRCA), head and neck squamous cell carcinoma (HNSCC), pancreatic adenocarcinoma (PAAD), lung squamous cell carcinoma (LUSC) or lung adenocarcinoma (LUAD). The bronchioid subtype can be indicative of a positive anti-folate predictive response signature (AF-PRS (+)). A non-bronchioid subtype (i.e., squamoid subtype in combination with a magnoid subtype) can be indicative of a negative antifolate predictive response signature AF-PRS (−). A squamoid subtype alone can be indicative of a negative antifolate predictive response signature AF-PRS (−). A magnoid subtype alone can be indicative of a negative antifolate predictive response signature AF-PRS (−).

In some instances, a plurality of classifier genes of Table 1 are capable of identifying a bronchioid subtype or positive AF-PRS with a predictive success of at least about 70%, at least about 71%, at least about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, up to 100%.

In some instances, a plurality of classifier genes of Table 1 are capable of identifying a bronchioid subtype or positive AF-PRS with a sensitivity or specificity of at least about 70%, at least about 71%, at least about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, up to 100%.

The present invention also encompasses a system capable of distinguishing various anti-folate predictive response subtypes or signatures of a sample regardless of cancer type not detectable using current methods. This system c an be capable of processing a large number of subjects and subject variables such as expression profiles and other diagnostic criteria. The methods described herein can also be used for “pharmacometabonomics,” in analogy to pharmacogenomics, e.g., predictive of response to therapy. In this embodiment, subjects could be divided into “responders” and “nonresponders” using the expression profile as evidence of “response,” and features of the expression profile could then be used to target future subjects who would likely respond to a particular therapeutic course.

In one embodiment, provided herein is a system for determining an antifolate predictive response signature of a sample obtained from a subject suffering from cancer. The system can comprise: (a) one or more processors; and (b) one or more memories operatively coupled to at least one of the one or more processors and having instructions stored thereon that, when executed by at least one of the one or more processors, cause the system to (i) detect an expression level of each of a plurality of classifier biomarkers from Table 1; (ii) compare the expression levels of each of the plurality of classifier biomarkers from Table 1 to the expression levels of each of the plurality of classifier biomarkers from Table 1 in a control; and (iii) classify the sample as TRU, PP, or PI based on the results of the comparing step. In some cases, instead of classifying the sample as TRU, PP or PI, the sample can classify the sample as TRU (+) or TRU (−). In some cases, instead of classifying the sample as TRU, PP or PI, the sample can classify the sample as AF-PRS (+) or AF-PRS (−). The control can comprise at least one sample training set(s). The at least one sample training set can comprise expression data of the plurality of classifier biomarkers of Table 1 from a reference adenocarcinoma TRU (bronchioid) sample, expression data of the plurality of classifier biomarkers of Table 1 from a reference adenocarcinoma PP (magnoid) sample, expression data of the plurality of classifier biomarkers of Table 1 from a reference adenocarcinoma PI (squamoid) sample, or a combination thereof. The comparing step can comprise applying a statistical algorithm. The statistical algorithm can determine a correlation between the expression data obtained from the sample and the expression data from the at least one training set(s); and classifying the sample as a TRU, PP, or PI subtype (or alternatively, TRU (+) or TRU (−) OR AF-PRS (+) or AF-PRS (−)) based on the results of the statistical algorithm. In some cases, the expression level of each of the plurality of classifier biomarkers from Table 1 is detected at the nucleic acid level. In some cases, the nucleic acid level is RNA or cDNA. In some cases, the detecting the expression level can be performed using any method known in the art and/or provided herein such as, for example, by performing qRT-PCR. The detecting the expression level can be performed using a device that is part of the system or in communication with at least one of the one or more processors, wherein upon receipt of instructions sent by the at least one of the one or more processors, perform the detection of the expression levels. The plurality of classifier biomarkers from Table 1 can comprise at least 8 classifier biomarkers, at least 16 classifier biomarkers, at least 24 classifier biomarkers, at least 32 classifier biomarkers, at least 40 classifier biomarkers or at least 48 classifier biomarkers from Table 1. The plurality of classifier biomarkers of Table 1 can comprise at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the classifier biomarkers from Table 1. In some cases, the plurality of classifier biomarkers of Table 1 can comprise flgf, ctsh, sctr, cyp4b1, gpr116, adh1b, cbx7, hlf cep55, tpx2, bub1b, kif4a, ccnb2, kif14, melk, kif11 or any combination thereof. In some cases, the plurality of classifier biomarkers of Table 1 can comprise fgl1, pbk, hspd1, tdg, prc1, dusp4, gtpbp4, zwint, tlr2, cd74, hla-dpb1, hla-dpa1, hla-dra, itgb2, fas, hla-drb1, plau, gbp1, dse, ccdc109b, tgfbi, cxcl10, lgals1, tubb6, gjb1, rap1gap, cacna2d2, selenbp1, tfcp2l1, sorbs2, unc13b, tacc2 or any combination thereof. In some cases, the plurality of classifier biomarkers of Table 1 can comprise all the classifier biomarkers from Table 1. In some cases, the TRU subtype is indicative of a positive antifolate predictive response signature, wherein the positive antifolate predictive response signature selects the patient for treatment with an antifolate agent. In some cases, the anti-folate agent is selected from pemetrexed, methotrexate, trimetrexate, lometrexol, raltitrexed and nolatrexed. In some cases, the antifolate agent is pemetrexed. In some cases, the antifolate agent is raltitrexed. In some cases, the cancer the patient is suffering from is selected from bladder cancer, breast cancer, pancreatic adenocarcinoma, lung adenocarcinoma, lung squamous cell carcinoma, and head and neck adenocarcinoma.

The expression profile can be used in combination with other diagnostic methods including histochemical, immunohistochemical, cytologic, immunocytologic, and visual diagnostic methods including histologic or morphometric evaluation of lung tissue.

In various embodiments of the AF-PRS, the expression profile derived from sample obtained from a subject is compared to a reference expression profile. A “reference expression profile” can be a profile derived from the subject prior to treatment or therapy; can be a profile produced from the subject sample at a particular time point (usually prior to or following treatment or therapy but can also include a particular time point prior to or following diagnosis of cancer); or can be derived from a healthy individual or a pooled reference from healthy individuals. As alluded to herein, in some cases, a reference expression profile can be for the bronchioid subtype of lung adenocarcinoma (LUAD) or one or a combination of both of the non-bronchioid sub-types of LUAD.

The reference expression profile can be compared to a test expression profile. A “test expression profile” can be derived from the same subject as the reference expression profile except at a subsequent time point (e.g., one or more days, weeks or months following collection of the reference expression profile) or can be derived from a different subject. In summary, any test expression profile of a subject can be compared to a previously collected profile from a subject that has a bronchioid (TRU), magnoid (PP), or squamoid (PI) subtype.

In general, the methods provided herein are used to classify a sample (e.g., tumor sample) obtained from a subject suffering from or suspected of suffering from a cancer as akin or similar to a particular subtype of lung adenocarcinoma (LUAD). The subtype of LUAD for the sample obtained from the subject suffering from or suspected of suffering from a cancer can indicate whether or not said subject is predicted to be responsive to treatment with anti-folate agents or not. If the sample possesses a gene expression profile similar to an expression profile of a control sample from a subject known to have a bronchioid subtype of LUAD, said subject can be predicted to be responsive to treatment with an anti-folate agent. The anti-folate agent can be any anti-folate agent known in the art and/or provided herein. The cancer can be any cancer known in the art and/or provided herein. In one embodiment, the method comprises measuring, detecting or determining an expression level of at least one or a plurality of the classifier biomarkers of Table 1 in the sample obtained from the subject. As provided herein, the number of classifiers of Table 1 whose expression level can be assessed or measured in a method for determining an anti-folate predictive response signature in a sample as provided herein can be all of the classifiers found in Table 1 or a subset thereof (i.e., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 or 48 classifier biomarkers from Table 1). As provided herein, the number of classifiers of Table 1 whose expression level can be assessed or measured in a method for determining an anti-folate predictive response signature in a sample as provided herein can comprises about 10%, 15%, 20%, 25%, 30%, 35%, 40%, 45%, 50%, 55%, 60%, 65%, 70%, 75%, 80%, 85%, 90%, 95% or 99% of the classifiers from Table 1. As provided herein, the number of classifiers of Table 1 whose expression level can be assessed or measured in a method for determining an anti-folate predictive response signature in a sample as provided herein can comprises at least 10%, 15%, 20%, 25%, 30%, 35%, 40%, 45%, 50%, 55%, 60%, 65%, 70%, 75%, 80%, 85%, 90%, 95% or 99% of the classifiers from Table 1. As provided herein, the number of classifiers of Table 1 whose expression level can be assessed or measured in a method for determining an anti-folate predictive response signature in a sample as provided herein can comprises at most 10%, 15%, 20%, 25%, 30%, 35%, 40%, 45%, 50%, 55%, 60%, 65%, 70%, 75%, 80%, 85%, 90%, 95% or 99% of the classifiers from Table 1. In one embodiment the subset of classifiers of Table 1 can comprise flgf, ctsh, sctr, cyp4b1, gpr116, adh1b, cbx7, hlf cep55, tpx2, bub1b, kif4a, ccnb2, kif14, melk, kif11 or any combination or subset thereof. In one embodiment the subset of classifiers of Table 1 can comprise fgl1, pbk, hspd1, tdg, prc1, dusp4, gtpbp4, zwint, tlr2, cd74, hla-dpb1, hla-dpa1, hla-dra, itgb2, fas, hla-drb1, plau, gbp1, dse, ccdc109b, tgfbi, cxcl10, lgals1, tubb6, gjb1, rap1gap, cacna2d2, selenbp1, tfcp2l1, sorbs2, unc13b, tacc2 or any combination thereof.

The sample for the detection or differentiation methods described herein can be a sample obtained from a subject that has been previously determined or diagnosed as suffering from a particular cancer. The previous diagnosis can be based on a histological analysis. The histological analysis can be performed by one or more pathologists. The sample can be any sample type known in the art and/or provided herein such as, for example a formalin-fixed, paraffin-embedded (FFPE) tissue sample, fresh or a frozen tissue sample, an exosome, wash fluids, cell pellets, or a bodily fluid obtained from the patient. In some cases, the bodily fluid is blood or fractions thereof, urine, saliva, or sputum.

### Overview-Proliferation Signature

The present invention also provides kits, compositions and methods for identifying or detecting cell proliferation in a tumor obtained from a subject. That is, the methods can be useful for molecularly defining proliferation. The methods provide assessment of proliferation in a sample (e.g., tumor sample) that can be prognostic for patients suffering from or suspected of suffering from a myriad of cancers. The methods also provide assessment of proliferation in a sample (e.g., tumor sample) that can be predictive for a therapeutic response. The therapeutic response can include chemotherapy, immunotherapy, surgical intervention or radiotherapy.

In one embodiment, the assessment of proliferation or the proliferation status of a sample (e.g., tumor sample) is determined by measuring, detecting or evaluating expression levels of one a plurality of classifier genes or biomarkers in one or more subject samples at the nucleic acid or protein level. In one embodiment, the one or more of the plurality of classifier genes or biomarkers is selected from the classifier biomarkers found in Table 2. In another embodiment, the assessing of proliferation includes detecting expression levels of at most, at least or about 2, 3, 4, 5, 6, 7, 8, 9, 10, 11, 12, 13, 14, 15, 16, 17, 18, 19, 20, 21, 22, 23, 24 or 26 of the classifier biomarkers of Table 2 at the nucleic acid level or protein level. For example, in one embodiment, from about 2 to about 5, from about 5 to about 10, from about 5 to about 15, from about 5 to about 20, from about 5 to about 25, from about 5 to about 26, from about 10 to about 15, from about 10 to about 20, from about 10 to about 25, from about 10 to about 26, from about 15 to about 20, from about 15 to about 25, from about 15 to about 26 of the biomarkers in Table 2 are detected at the nucleic acid or protein level in a method to assess proliferation in a sample. In another embodiment, the assessing of proliferation includes detecting expression levels of at least 10%, 15%, 20%, 25%, 30%, 35%, 40%, 45%, 50%, 55%, 60%, 65%, 70%, 75%, 80%, 85%, 90%, 95% or 99% of the classifiers from Table 2. In another embodiment, the assessing of proliferation includes detecting expression levels of about 10%, 15%, 20%, 25%, 30%, 35%, 40%, 45%, 50%, 55%, 60%, 65%, 70%, 75%, 80%, 85%, 90%, 95% or 99% of the classifiers from Table 2. In another embodiment, the assessing of proliferation includes detecting expression levels of at most 10%, 15%, 20%, 25%, 30%, 35%, 40%, 45%, 50%, 55%, 60%, 65%, 70%, 75%, 80%, 85%, 90%, 95% or 99% of the classifiers from Table 2. In some embodiments, the assessing proliferation includes detecting expression levels of all of the classifier biomarkers of Table 2 at the nucleic acid level or protein level. The expression levels of the one or more of the plurality of classifier biomarkers as determined for a sample obtained from a subject can be referred to as the proliferation profile or the proliferation signature of said sample. In one embodiment, the nucleic acid expression level is determined in the methods provided herein for assessing proliferation. Further to this embodiment, the nucleic acid expression level of each classifier gene from the plurality of classifier genes (e.g., Table 2) is log 2-transformed.

In one embodiment, the expression level (e.g., nucleic acid or protein) of each of the classifier biomarkers (e.g., from Table 2) can be normalized. “Normalization” may be used to remove sample-to-sample variation. In embodiments where microarray data is used to detect expression levels of the classifier biomarker(s) (e.g., from Table 2), the process of normalization aims to remove systematic errors by balancing the fluorescence intensities of the two labeling dyes. The dye bias can come from various sources including differences in dye labeling efficiencies, heat and light sensitivities, as well as scanner settings for scanning two channels. Some commonly used methods for calculating normalization factor can include: (i) global normalization that uses all genes on the array; (ii) housekeeping genes normalization that uses constantly expressed housekeeping/invariant genes; and (iii) internal controls normalization that uses known amount of exogenous control genes added during hybridization (Quackenbush Nat. Genet. 32 (Suppl.), 496-501 (2002)). In one embodiment, expression levels of the classifier gene(s) disclosed herein (e.g., Table 2) can be normalized to control housekeeping genes. For example, the housekeeping genes described in U.S. Patent Publication 2008/0032293, which is herein incorporated by reference in its entirety, can be used for normalization. Exemplary housekeeping genes include mrpl19, psmc4, sf3a1, pum1, actb, gapd, gusb, rplpo, and tfrc. It will be understood by one of skill in the art that the methods disclosed herein are not bound by normalization to any particular housekeeping genes, and that any suitable housekeeping gene(s) known in the art can be used.

Many normalization approaches are possible, and they can often be applied at any of several points in the analysis. In one embodiment, microarray data is normalized using the LOWESS method, which is a global locally weighted scatter plot smoothing normalization function. In another embodiment, qPCR (or qRT-PCR) data is normalized to the geometric mean of set of multiple housekeeping genes. In yet another embodiment, qPCR (or qRT-PCR) data is normalized by first normalizing the raw Ct values to gene specific technical controls followed by normalizing to housekeeping genes inserted as sample controls. Said housekeeping genes can be those provided herein.

In one embodiment, following the determination of the proliferation signature or proliferation profile of a sample, a proliferation score for the sample is calculated. In one embodiment, the proliferation score for a sample is determined by averaging the normalized expression estimates for each classifier in said sample. In another embodiment, the proliferation score for a sample is determined by calculating the average log 2 transformed expression level across all of the classifier genes from Table 2 whose expression level was determined. As provided herein, the number of classifier genes of Table 2 whose expression level can be determined in a method for assessing proliferation in a sample as provided herein can be all of the classifiers found in Table 2 or a subset thereof (i.e., 1, 2, 3, 4, 5, 6, 7, 8, 9, 10, 11, 12, 13, 14, 15, 16, 17, 18, 19, 20, 21, 22, 23, 24 or 25 classifier biomarkers from Table 2). In another embodiment, the assessing of proliferation includes detecting expression levels of about 10%, 15%, 20%, 25%, 30%, 35%, 40%, 45%, 50%, 55%, 60%, 65%, 70%, 75%, 80%, 85%, 90%, 95% or 99% of the classifiers from Table 2. In another embodiment, the assessing of proliferation includes detecting expression levels of at least 10%, 15%, 20%, 25%, 30%, 35%, 40%, 45%, 50%, 55%, 60%, 65%, 70%, 75%, 80%, 85%, 90%, 95% or 99% of the classifiers from Table 2. In another embodiment, the assessing of proliferation includes detecting expression levels of at most 10%, 15%, 20%, 25%, 30%, 35%, 40%, 45%, 50%, 55%, 60%, 65%, 70%, 75%, 80%, 85%, 90%, 95% or 99% of the classifiers from Table 2.

In one embodiment, the “proliferation profile” or a “proliferation signature” associated with the gene cassettes or classifier genes described herein (e.g., Table 2) can be useful for distinguishing between proliferative and non-proliferative samples. Further to this embodiment, the proliferation profile or signature as determined for a sample obtained from a subject can be compared to a control sample. In one embodiment, the control sample is a sample obtained from a healthy subject not suspected of being proliferative. Further to this embodiment, the proliferation profile or signature obtained from the subject using the methods provided herein is compared to the proliferation profile or signature for the sample obtained from the healthy subject using the methods provided herein. If the proliferation signatures or profiles are identical or substantially similar, then the sample obtained from the subject is not suspected of being proliferative. If the proliferation signatures or profiles are different or substantially different, then this may indicate that the sample obtained from the subject is proliferative and/or may warrant further examination or analysis. In another embodiment, the control sample is a proliferative sample obtained from a subject known to be experiencing proliferation. Further to this embodiment, the proliferation profile or signature obtained from the subject using the methods provided herein is compared to the proliferation profile or signature for the proliferative sample using the methods provided herein. If the proliferation signatures or profiles are identical or substantially similar, then the sample obtained from the subject is suspected of being proliferative and/or may warrant further examination or analysis. If the proliferation signatures or profiles are different or substantially different, then this may indicate that the sample obtained from the subject is not proliferative.

In another embodiment, the proliferation score for a sample obtained from a subject as calculated using the methods provided herein can be useful for distinguishing between proliferative and non-proliferative samples. Further to this embodiment, the proliferation score as determined for a sample obtained from a subject can be compared to a control sample. In one embodiment, the control sample is a sample obtained from a healthy subject not suspected of being proliferative. Further to this embodiment, the proliferation score obtained from the subject using the methods provided herein is compared to the proliferation score for the sample obtained from the healthy subject using the methods provided herein. If the proliferation scores are identical or substantially similar, then the sample obtained from the subject is not suspected of being proliferative. If the proliferation scores are different or substantially different, then this may indicate that the sample obtained from the subject is proliferative and/or may warrant further examination or analysis. In another embodiment, the control sample is a proliferative sample obtained from a subject known to be experiencing proliferation. Further to this embodiment, the proliferation score obtained from the subject using the methods provided herein is compared to the proliferation score for the proliferative sample using the methods provided herein. If the proliferation scores are identical or substantially similar, then the sample obtained from the subject is suspected of being proliferative and/or may warrant further examination or analysis. If the proliferation scores are different or substantially different, then this may indicate that the sample obtained from the subject is not proliferative.

TABLE 2
Classifier Biomarkers for Proliferation Signature

| | | GenBank | SEQ |
|---|---|---|---|
| Gene | | Accession | ID |
| Symbol | Gene Name | Number* | NO: |
| TPX2 | TPX2, microtubule nucleation factor | NM_012112.4 | 49 |
| DLGAP5 | DLG associated protein 5 | NM_014750.4 | 50 |
| HJURP | Holliday junction recognition protein | NM_018410.4 | 51 |
| KIF4A | kinesin family member 4A | NM_012310.4 | 52 |
| KIF2C | kinesin family member 2C | NM_006845.3 | 53 |
| PLK1 | polo like kinase 1 | NM_005030.5 | 54 |
| MELK | maternal embryonic leucine zipper kinase | NM_014791.3 | 55 |
| CCNB2 | cyclin B2 | NM_004701.3 | 56 |
| BUB1 | BUB1 mitotic checkpoint serine/threonine kinase | NM_004336.4 | 57 |
| KIF23 | kinesin family member 23 | NM_138555.4 | 58 |
| UBE2C | ubiquitin conjugating enzyme E2 C | NM_007019.3 | 59 |
| KIF20A | kinesin family member 20A | NM_005733.2 | 60 |
| TROAP | trophinin associated protein | NM_005480.3 | 61 |
| AURKB | aurora kinase B | NM_004217.3 | 62 |
| RRM2 | ribonucleotide reductase regulatory subunit M2 | NM_001165931.1 | 63 |
| MYBL2 | MYB proto-oncogene like 2 | NM_002466.3 | 64 |
| MKI67 | marker of proliferation Ki-67 | NM_002417.4 | 65 |
| CDC20 | cell division cycle 20 | NM_001255.2 | 66 |
| CEP55 | centrosomal protein 55 | NM_018131.4 | 67 |
| TOP2A | DNA topoisomerase II alpha | NM_001067.3 | 68 |
| BIRC5 | baculoviral IAP repeat containing 5 | NM_001168.2 | 69 |
| ASPM | abnormal spindle microtubule assembly | NM_018136.4 | 70 |
| ESPL1 | extra spindle pole bodies like 1, separase | NM_012291.4 | 71 |
| KIF18B | kinesin family member 18B | NM_001265577.1 | 72 |
| IQGAP3 | IQ motif containing GTPase activating protein 3 | NM_178229.4 | 73 |
| EPR1 | proline-rich extensin-like protein | XM_017020903.1 | 74 |

*Each GenBank Accession Number is a representative or exemplary GenBank Accession Number for the listed gene and is herein incorporated by reference in its entirety for all purposes. Further, each listed representative or exemplary accession number should not be construed to limit the claims to the specific accession number.

In one embodiment, the determination of the proliferation profile or signature or proliferation score of a sample obtained from a subject by detecting or measuring the expression levels of one or a plurality of classifiers from Table 2 can be combined with additional methods for assessing proliferation. Additional methods for assessing proliferation can be any method known in the art such as additional gene expression-based proliferation signatures and/or histochemical analysis of a tumor tissue sample for known proliferation markers. Examples of gene expression methods for assessing proliferation that can be used alone or in combination with detecting the expression of one or a plurality of classifiers from Table 2 can be the PAM50 proliferation signature disclosed in Nielsen T O et al., Clin Cancer Res. 2010 Nov. 1; 16(21):5222-32 or the gene proliferation signature disclosed in US20160115551, each of which is hereby incorporated by reference. Examples of known proliferation markers that can be histochemically analyzed can be selected from Ki-67 (see Dowsett M, Nielsen T O, A'Hern R, Bartlett J, Coombes R C, Cuzick J, et al. Assessment of Ki67 in breast cancer: recommendations from the International Ki67 in Breast Cancer working group. J Natl Cancer Inst. 2011; 103(22):1656-64), Estrogen Receptor (ER) (see Hammond M E, Hayes D F, Wolff A C, Mangu P B, Temin S. American society of clinical oncology/college of American pathologists' guideline recommendations for immunohistochemical testing of estrogen and progesterone receptors in breast cancer. J Oncol Pract. 2010; 6(4):195-7), CD31 and/or Her2 (see Wolff A C, Hammond M E, Hicks D G, Dowsett M, McShane L M, Allison K H, et al. Recommendations for human epidermal growth factor receptor 2 testing in breast cancer: American Society of Clinical Oncology/College of American Pathologists clinical practice guideline update. J Clin Oncol. 2013; 31(31):3997-4013), each of which is hereby incorporated by reference.

In one embodiment, provided herein is a system for determining a proliferation score in a subject. The system can comprise: (a) one or more processors; and (b) one or more memories operatively coupled to at least one of the one or more processors and having instructions stored thereon that, when executed by at least one of the one or more processors, cause the system to (i) determine a proliferation score of a sample obtained from the subject, wherein the determining the proliferation score comprises: (a) measuring a nucleic acid expression level of at least five classifier genes from a plurality of classifier genes in the sample obtained from the cancer patient, wherein the plurality of classifier genes consists of only tpx2, dlgap5, hjurp, kif4a, kif2c, plk1, melk, ccnb2, bub1, kif23, ube2c, kif20a, troap, aurkb, rrm2, mybl2, mki67, cdc20, cep55, top2a, birc5, aspm, espl1, kif18b, iqgap3 and epr1; and (b) calculating a mean nucleic acid expression level across the at least five classifier biomarkers from the plurality of classifier biomarkers, wherein the mean nucleic acid expression level across the at least five classifier biomarkers from the plurality of classifier biomarkers represents the proliferation score; (ii) determine a proliferation score of a control sample, wherein the determining the proliferation score comprises: (a) measuring a nucleic acid expression level of at least five classifier genes from a plurality of classifier genes in the control sample, wherein the plurality of classifier genes consists of only tpx2, dlgap5, hjurp, kif4a, kif2c, plk1, melk, ccnb2, bub1, kif23, ube2c, kif20a, troap, aurkb, rrm2, mybl2, mki67, cdc20, cep55, top2a, birc5, aspm, espl1, kif18b, iqgap3 and epr1; and (b) calculating a mean nucleic acid expression level across the at least five classifier biomarkers from the plurality of classifier biomarkers, wherein the mean nucleic acid expression level across the at least five classifier biomarkers from the plurality of classifier biomarkers represents the proliferation score; and (iii) compare the proliferation score of the sample obtained from the subject to the proliferation score of the control sample, wherein an elevated proliferation score in the sample obtained from the subject is indicative of a poor disease outcome for the subject, wherein the subject suffers from or is suspected of suffering from a cancer.

### Metastatic Disease

In one embodiment, the determining the expression of one or a plurality of biomarkers from Table 2 is used to determine the presence of metastatic disease in a subject. The subject can be suffering from or suspected of suffering from a primary cancer. The primary cancer can be any cancer known in the art and/or provided herein. In one embodiment, the subject is suffering from or suspected of suffering from a primary cancer selected from KIRP, BRCA, THCA, BLCA, PRAD, KICH, CESC, KIRC, LIHC, LGG, SARC, LUAD, COAD, HNSC. UCEC, GBM. ESCA. STAD, OV or READ.

The method for determining the presence of metastatic disease in said subject can comprise or consist of measuring an expression level of at least five classifier genes from a plurality of classifier genes in a first sample obtained from the subject, wherein the expression level of the at least five classifier genes represents a proliferation signature of the first sample, measuring the expression level of the same at least five classifier genes from the plurality of classifier genes in a second sample, wherein the expression level of the at least five classifier genes represents a proliferation signature of the second sample, and determining existence of a correlation between the proliferation signature of the first sample and the proliferation signature of the second sample. The expression level can be normalized as provided herein, such as, for example, normalizing expression of the classifier genes by using expression levels from one or more reference or housekeeping genes.

In one embodiment, prior to determining the existence of a correlation between the proliferation signature of the first sample and the proliferation signature of the second sample, the method comprises or consist of determining a proliferation score for the first sample and the second sample. Determining the proliferation score can comprise determining a mean nucleic acid expression level for the at least five classifier biomarkers from the plurality of classifier biomarkers for the first sample and the second sample. Determining the existence of a correlation can entail determining the existence of a correlation between the proliferation score of the first sample and the proliferation score of the second sample.

The correlation between the first sample and the second sample can be performed in a various ways. The correlation can be determined using any statistical test or algorithm known in the art that is appropriate for such an analysis. In some cases, a correlation coefficient is determined that is a measure of the similarity of dissimilarity of the first sample with said second sample. A number of different coefficients can be used for determining a correlation between the expression level in the first sample from the subject and the second sample. In some cases, the methods for determining a correlation coefficient are parametric methods, which assume a normal distribution of the data. One of these methods can be the Pearson product-moment correlation coefficient, which can be obtained by dividing the covariance of the two variables by the product of their standard deviations. Other methods can comprise cosine-angle, un-centered correlation and, more preferred, cosine correlation (Fan et al., Conf Proc IEEE Eng Med Biol Soc. 5:4810-3 (2005)). In some cases, the methods for determining a correlation coefficient are non-parametric methods such as, for example, methods for determining a Kendall correlation or a Spearman correlation.

In one embodiment, the second sample is from a different area of the subject's body as the first sample and the existence of a positive correlation is indicative of the likelihood of metastatic disease in the patient. Further to this embodiment, the existence of a negative correlation is indicative of the likelihood of the absence of metastatic disease in the patient. In another embodiment, the second sample is obtained from a control subject that does not have metastatic disease and the existence of a negative correlation is indicative of the likelihood of metastatic disease in the patient. Further to this embodiment, the existence of a positive correlation is indicative of the likelihood of the absence of metastatic disease in the patient. Further to this embodiment, the second sample obtained from the control subject can be from the same area of the body as the first sample. In yet another embodiment, the second sample is obtained from a control subject that does have metastatic disease and the existence of a positive correlation is indicative of the likelihood of metastatic disease in the patient. Further to this embodiment, the existence of a negative correlation is indicative of the likelihood of the absence of metastatic disease in the patient. Further to this embodiment, the second sample obtained from the control subject can be from the same area of the body as the first sample.

The first and/or second sample can be a formalin-fixed, paraffin-embedded (FFPE) tissue sample, fresh or a frozen tissue sample, an exosome, wash fluids, cell pellets, or a bodily fluid obtained from the subject. In one embodiment, the first sample and the second sample is an FFPE tissue sample. In another embodiment, the first sample and the second sample is a fresh frozen tissue sample.

In one embodiment, the method for determining the presence of metastatic disease as provided herein further comprises determining a subtype of the sample obtained from the subject. The subtype can be determined via histological examination of the sample. The subtype can be determined via gene expression analysis of the sample. The gene expression analysis of the sample is performed using a gene expression sub-typer that is publicly available and/or provided herein. The gene expression-based cancer subtyping can be determined using gene signatures known in the art for specific types of cancer. In one embodiment, the cancer is lung cancer, and the gene signature is selected from the gene signatures found in WO2017/201165, WO2017/201164, US20170114416 or U.S. Pat. No. 8,822,153, each of which is herein incorporated by reference in their entirety. In one embodiment, the cancer is head and neck squamous cell carcinoma (HNSCC) and the gene signature is selected from the gene signatures found in PCT/US18/45522 or PCT/US18/48862, each of which is herein incorporated by reference in their entirety. In one embodiment, the cancer is breast cancer, and the gene signature is the PAM50 subtyper found in Parker J S et al., (2009) Supervised risk predictor of breast cancer based on intrinsic subtypes. J Clin Oncol 27:1160-1167, which is herein incorporated by reference in its entirety.

Correlation can be a bivariate analysis that measures the strength of association between two variables and the direction of the relationship. In some cases, said correlation of the first sample with the second sample can be used to produce an overall similarity score for the set of classifier genes (e.g., from Table 2) that are used. A similarity score can be a measure of the average correlation of the expression levels of the one or plurality of classifier genes (e.g., from Table 2) in the first sample from the subject and the second sample. Said similarity score can be a numerical value between +1, indicative of a high correlation between the expression levels of the one or plurality of classifier genes (e.g., from Table 2) in the first sample from the subject and the second sample, and −1, which is indicative of an inverse correlation (van't Veer et al., Nature 415: 484-5 (2002)).

In one embodiment, a similarity score is determined as provided herein and an arbitrary threshold is determined for said similarity score. In embodiments where the second sample is from another or different part of the subject's body, a similarity score at or above the threshold can indicate a low risk of metastatic disease, while a similarity score below said threshold can be indicative of metastatic disease. In embodiments where the second sample is from a subject that does not have metastatic disease, first samples that score below said threshold are indicative of an increased risk of metastasis, while first samples that score at or above said threshold are indicative of a low risk of metastasis. In embodiments wherein the second sample is from a subject that does have metastatic disease, first samples that score below said threshold are indicative of a decreased risk of metastasis, while first samples that score at or above said threshold are indicative of a high risk of metastasis. In yet another embodiment, the method for determining the presence of metastatic disease comprises determining a proliferation signature or score and a subtype of a first sample obtained from a subject as well as a proliferation signature or score and a subtype of a second sample obtained from another or different part of the subject's body and calculating a similarity score of the two samples using the methods provided herein. Further to this embodiment, a similarity score at or above the threshold in combination with a similar subtype between the first and second sample can be indicative of metastatic disease, while a similarity score that is below the threshold in combination with different subtypes between the first and second sample can indicate the absence of metastatic disease.

In some cases, the method for determining the presence of metastatic disease comprises or consists of measuring the expression level is of at least 10, 15, 20 or 25 classifier genes from the plurality of classifier genes. In some cases, the method comprises or consist of measuring the expression level of all of the classifier genes from the plurality of classifier genes. In one embodiment, the plurality of classifier genes are the classifier genes found in Table 2. In another embodiment, the plurality of classifier genes are about 10%, 15%, 20%, 25%, 30%, 35%, 40%, 45%, 50%, 55%, 60%, 65%, 70%, 75%, 80%, 85%, 90%, 95% or 99% of the classifier genes found in Table 2. In another embodiment, the plurality of classifier genes are at least 10%, 15%, 20%, 25%, 30%, 35%, 40%, 45%, 50%, 55%, 60%, 65%, 70%, 75%, 80%, 85%, 90%, 95% or 99% of the classifier genes found in Table 2. In another embodiment, the plurality of classifier genes are at most 10%, 15%, 20%, 25%, 30%, 35%, 40%, 45%, 50%, 55%, 60%, 65%, 70%, 75%, 80%, 85%, 90%, 95% or 99% of the classifier genes found in Table 2.

The expression level can be a nucleic acid or protein expression level. The nucleic acid or protein expression level can be measured using any method known in the art and/or provided herein. In one embodiment, the method of determining the presence of metastatic disease as provided herein entails measuring a nucleic acid expression level. The nucleic acid expression level can be measured using an amplification, sequencing or hybridization assay. The amplification, hybridization and/or sequencing assay can comprise performing quantitative real time reverse transcriptase polymerase chain reaction (qRT-PCR), RNA-seq, microarrays, gene chips, nCounter Gene Expression Assay, Serial Analysis of Gene Expression (SAGE), Rapid Analysis of Gene Expression (RAGE), nuclease protection assays, Northern blotting, nCounter DX Analysis System or any other equivalent gene expression detection techniques. In one embodiment, the nucleic acid expression level is detected by performing RNA-seq.

Further to any of the embodiments related to determining the presence of metastatic disease in a subject, the method can further comprises determining a level and/or activity of at least one additional marker involved in cell proliferation and mitosis in the first and second samples. An increase in the additional marker in the first and second samples can be further indicative of metastasis of the type of cancer. The one additional marker can be any marker known in the art and/or provided herein as playing a role in proliferation or mitosis. The additional marker can be selected from the group consisting of Ki-67, CD31, KIFC1 (kinesin family member C1), KIF2C (kinesin family member 2C), KIF14 (kinesin family member 14), CCNB2 (cyclin B2), SIL (SCL-TAL1 interrupting locus) and TNPO1 (transportin I). In one embodiment, the additional marker is Ki67 or CD31.

In another embodiment, provided herein is a system for determining metastatic disease in a subject. The system can comprise: (a) one or more processors; and (b) one or more memories operatively coupled to at least one of the one or more processors and having instructions stored thereon that, when executed by at least one of the one or more processors, cause the system to: (i) measure a nucleic acid expression level of at least five classifier genes from a plurality of classifier genes in a first sample obtained from the subject, wherein the plurality of classifier genes consists of only tpx2, dlgap5, hjurp, kif4a, kif2c, plk1, melk, ccnb2, bub1, kif23, ube2c, kif20a, troap, aurkb, rrm2, mybl2, mki67, cdc20, cep55, top2a, birc5, aspm, espl1, kif18b, iqgap3 and epr1, wherein the nucleic acid expression level of the at least five classifier genes represents a proliferation signature of the first sample; (ii) measure the nucleic acid expression level of the same at least five classifier genes from the plurality of classifier genes in a second sample, wherein the nucleic acid expression level of the at least five classifier genes represents a proliferation signature of the second sample; and (iii) determine existence of a correlation between the proliferation signature of the first sample and the proliferation signature of the second sample, wherein the existence of a correlation is indicative of the likelihood of metastatic disease in the subject. In some cases, the system further comprises one or more devices that are configured to perform the measuring and/or determining steps outlined herein.

### Measuring Biomarkers Expression Levels

In some embodiments, the method for determining an anti-folate predictive response signature or subtyping (AF-PRS) includes detecting expression levels of one or more classifier biomarkers from the set of classifier markers found in Table 1. In some embodiments, the detecting includes all of the classifier biomarkers of Table 1 at the nucleic acid level or protein level. In another embodiment, a single or a subset of the classifier biomarkers of Table 1 are detected, for example, from about 8 to about 16. For example, in one embodiment, from about 5 to about 10, from about 5 to about 15, from about 5 to about 20, from about 5 to about 25, from about 5 to about 30, from about 5 to about 35, from about 5 to about 40, from about 5 to about 45, from about 5 to about 48 of the biomarkers in Table 1 are detected in a method to determine the AF-PRS. In another embodiment, each of the biomarkers from Table 1 is detected in a method to determine the AF-PRS. In some cases, the subset of classifiers of Table 1 can comprise about 10%, 15%, 20%, 25%, 30%, 35%, 40%, 45%, 50%, 55%, 60%, 65%, 70%, 75%, 80%, 85%, 90%, 95% or 99% of the classifiers from Table 1. In some cases, the subset of classifiers of Table 1 can comprise at least 10%, 15%, 20%, 25%, 30%, 35%, 40%, 45%, 50%, 55%, 60%, 65%, 70%, 75%, 80%, 85%, 90%, 95% or 99% of the classifiers from Table 1. In some cases, the subset of classifiers of Table 1 can comprise at most 10%, 15%, 20%, 25%, 30%, 35%, 40%, 45%, 50%, 55%, 60%, 65%, 70%, 75%, 80%, 85%, 90%, 95% or 99% of the classifiers from Table 1. In one embodiment the subset of classifiers of Table 1 can comprise flgf, ctsh, sctr, cyp4b1, gpr116, adh1b, cbx7, hlf cep55, tpx2, bub1b, kif4a, ccnb2, kif14, melk, kif11 or any combination or subset thereof. In one embodiment the subset of classifiers of Table 1 can comprise fgl1, pbk, hspd1, tdg, prc1, dusp4, gtpbp4, zwint, tlr2, cd74, hla-dpb1, hla-dpa1, hla-dra, itgb2, fas, hla-drb1, plau, gbp1, dse, ccdc109b, tgfbi, cxcl10, lgals1, tubb6, gjb1, rap1gap, cacna2d2, selenbp1, tfcp2l1, sorbs2, unc13b, tacc2 or any combination thereof.

In some embodiments, the method for determining proliferation includes detecting expression levels of one or more classifier biomarkers from the set of classifier markers found in Table 2. In some embodiments, the detecting includes all of the classifier biomarkers of Table 2 at the nucleic acid level or protein level. In another embodiment, a single or a subset of the classifier biomarkers of Table 2 are detected, for example, from about 5 to about 20. For example, in one embodiment, from about 2 to about 4, from about 2 to about 8, from about 2 to about 16, from about 2 to about 20 or from about 2 to about 26 of the biomarkers in Table 2 are detected in a method to determine proliferation. In another embodiment, each of the biomarkers from Table 2 is detected in a method to determine proliferation. In another embodiment, the subset is about 10%, 15%, 20%, 25%, 30%, 35%, 40%, 45%, 50%, 55%, 60%, 65%, 70%, 75%, 80%, 85%, 90%, 95% or 99% of the classifiers from Table 2. In another embodiment, the subset is at least 10%, 15%, 20%, 25%, 30%, 35%, 40%, 45%, 50%, 55%, 60%, 65%, 70%, 75%, 80%, 85%, 90%, 95% or 99% of the classifiers from Table 2. In another embodiment, the subset is at most 10%, 15%, 20%, 25%, 30%, 35%, 40%, 45%, 50%, 55%, 60%, 65%, 70%, 75%, 80%, 85%, 90%, 95% or 99% of the classifiers from Table 2.

The detecting, determining or measuring in any of the methods provided herein can be performed by any suitable technique including, but not limited to, RNA-seq, a reverse transcriptase polymerase chain reaction (RT-PCR), a microarray hybridization assay, or another hybridization assay, e.g., a NanoString assay for example, with primers and/or probes specific to the classifier biomarkers, and/or the like. In some cases, the primers useful for the amplification methods (e.g., RT-PCR or qRT-PCR) are any forward and reverse primers suitable for binding to a classifier gene provided herein, such as the classifier biomarkers listed in Table 1 or Table 2.

In one embodiment, the measuring or detecting step for methods of determining an anti-folate predictive response signature or subtype (AF-PRS) provided herein is at the nucleic acid level by performing RNA-seq, a reverse transcriptase polymerase chain reaction (RT-PCR) or a hybridization assay with oligonucleotides that are substantially complementary to portions of cDNA molecules of the at least one or plurality of classifier biomarker(s) of Table 1 under conditions suitable for RNA-seq, RT-PCR or hybridization and obtaining expression levels of the at least one or plurality of classifier biomarkers based on the detecting step. The expression levels of the at least one or plurality of the classifier biomarkers are then compared to reference expression levels of the at least one or plurality of the classifier biomarker of Table 1 from at least one sample training set. The at least one sample training set can comprise, (i) expression levels of the at least one or a plurality of biomarker(s) from Table 1 from a sample that overexpresses the at least one or plurality of biomarker(s), (ii) expression levels from a reference squamoid (proximal inflammatory), bronchioid (terminal respiratory unit) or magnoid (proximal proliferative) sample, (iii) expression levels from an AF-PRS (+) sample or (iv) expression levels from an AF-PRS (−) sample. The sample can then be classified as a bronchioid or AF-PRS (+) or non-bronchioid or AF-PRS (−) subtype based on the results of the comparing step. In one embodiment, the comparing step can comprise applying a statistical algorithm which comprises determining a correlation between the expression data obtained from the sample obtained from the subject and the expression data from the at least one training set(s); and classifying the sample as a bronchioid or AF-PRS (+) or non-bronchioid or AF-PRS (−) subtype based on the results of the statistical algorithm. The statistical algorithm can entail finding the centroid to which the AF-PRS of the sample obtained from the subject is nearest from the centroids constructed from the expression data from the at least one training set, using any distance measure e.g., Euclidean distance or correlation. The centroids can be constructed using any method known in the art for generating centroids such as, for example, those found in Mullins et al. (2007) Clin Chem. 53(7):1273-9 or Dabney (2005) Bioinformatics 21(22):4148-4154 The AF-PRS of the sample obtained from subject can then be assigned based on the use of a classification to the nearest centroid (CLaNC) algorithm as applied to the expression data generated from the sample obtained from the subject and the centroid(s) constructed for the at least one training set. The CLaNC algorithm for use in the methods, compositions and kits provided herein can be the CLaNC algorithm implemented by the CLaNC software found in Dabney A R. ClaNC: Point-and-click software for classifying microarrays to nearest centroids. Bioinformatics. 2006; 22: 122-123 or equivalents or derivatives thereof.

In one embodiment, the measuring or detecting step for methods of determining an anti-folate predictive response signature or subtype (AF-PRS) provided herein comprises mixing the sample with one or more oligonucleotides that are substantially complementary to portions of cDNA molecules of the at least one or plurality of classifier biomarkers of Table 1 under conditions suitable for hybridization of the one or more oligonucleotides to their complements or substantial complements; detecting whether hybridization occurs between the one or more oligonucleotides to their complements or substantial complements; and obtaining hybridization values of the at least one or plurality of classifier biomarkers based on the detecting step. The hybridization values of the at least one or plurality of classifier biomarkers are then compared to reference hybridization value(s) from at least one sample training set. In some cases, the at least one sample training set comprises hybridization values from a reference bronchioid (TRU) adenocarcinoma and/or non-bronchioid (magnoid (PP) adenocarcinoma, and/or squamoid (PI) adenocarcinoma) sample. In some cases, the at least one sample training set comprises hybridization values from a reference AF-PRS (+) sample and/or an AF-PRS (−) sample. The sample is classified, for example, as being bronchioid or AF-PRS (+) or non-bronchioid or AF-PRS (−) based on the results of the comparing step.

In one embodiment, the measuring or detecting step for methods for assessing proliferation or determining proliferation score as provided herein is at the nucleic acid level by performing RNA-seq, a reverse transcriptase polymerase chain reaction (RT-PCR) or a hybridization assay with oligonucleotides that are substantially complementary to portions of cDNA molecules of the at least one classifier biomarker (such as the classifier biomarkers of Tables 1 or 2) under conditions suitable for RNA-seq, RT-PCR or hybridization and obtaining expression levels of the at least one classifier biomarkers based on the detecting step. The expression levels of the at least one of the classifier biomarkers are then compared to reference expression levels of the at least one of the classifier biomarker (such as the classifier biomarkers of Tables 1 or 2) from at least one sample training set. The comparison can be performed using any of the methods provided herein (e.g., CLaNC).

In methods for determining a proliferation signature or score as provided herein, the at least one sample training set can comprise, (i) expression levels of the at least one biomarker from a from a reference tumor sample that is proliferative, or (ii) expression levels from a non-proliferative sample and classifying the tumor sample as being proliferative or non-proliferative based on the results of the comparing step. In one embodiment, the comparing step can comprise applying a statistical algorithm which comprises determining a correlation between the expression data obtained from the tumor sample and the expression data from the at least one training set(s); and classifying the tumor sample as being proliferative or non-proliferative based on the results of the statistical algorithm.

In one embodiment, the measuring or detecting step for methods for assessing proliferation or determining proliferation score as provided herein comprises mixing the tumor sample with one or more oligonucleotides that are substantially complementary to portions of cDNA molecules of the at least one classifier biomarkers provided herein, such as the classifier biomarkers of Table 2 under conditions suitable for hybridization of the one or more oligonucleotides to their complements or substantial complements; detecting whether hybridization occurs between the one or more oligonucleotides to their complements or substantial complements; and obtaining hybridization values of the at least one classifier biomarkers based on the detecting step. The hybridization values of the at least one classifier biomarkers are then compared to reference hybridization value(s) from at least one sample training set. For example, the at least one sample training set comprises hybridization values from a reference proliferative tumor sample and/or reference non-proliferative sample. The tumor sample is classified, for example, as proliferative or non-proliferative based on the results of the comparing step.

The biomarkers described herein include RNA comprising the entire or partial sequence of any of the nucleic acid sequences of interest, or their non-natural cDNA product, obtained synthetically in vitro in a reverse transcription reaction. The term “fragment” is intended to refer to a portion of the polynucleotide that generally comprise at least 10, 15, 20, 50, 75, 100, 150, 200, 250, 300, 350, 400, 450, 500, 550, 600, 650, 700, 800, 900, 1,000, 1,200, or 1,500 contiguous nucleotides, or up to the number of nucleotides present in a full-length biomarker polynucleotide disclosed herein. A fragment of a biomarker polynucleotide will generally encode at least 15, 25, 30, 50, 100, 150, 200, or 250 contiguous amino acids, or up to the total number of amino acids present in a full-length biomarker protein of the invention.

In some embodiments, overexpression, such as of an RNA transcript or its expression product, is determined by normalization to the level of reference RNA transcripts or their expression products, which can be all measured transcripts (or their products) in the sample or a particular reference set of RNA transcripts (or their non-natural cDNA products). Normalization is performed to correct for or normalize away both differences in the amount of RNA or cDNA assayed and variability in the quality of the RNA or cDNA used. Therefore, an assay typically measures and incorporates the expression of certain normalizing genes, including well known housekeeping genes, such as, for example, GAPDH and/or β-Actin. Alternatively, normalization can be based on the mean or median signal of all of the assayed biomarkers or a large subset thereof (global normalization approach).

Isolated mRNA can be used in hybridization or amplification assays that include, but are not limited to, Southern or Northern analyses, PCR analyses and probe arrays, NanoString Assays. One method for the detection of mRNA levels involves contacting the isolated mRNA or synthesized cDNA with a nucleic acid molecule (probe) that can hybridize to the mRNA encoded by the gene being detected. The nucleic acid probe can be, for example, a cDNA, or a portion thereof, such as an oligonucleotide of at least 7, 15, 30, 50, 100, 250, or 500 nucleotides in length and sufficient to specifically hybridize under stringent conditions to the non-natural cDNA or mRNA biomarker of the present invention.

As explained above, in one embodiment, once the mRNA is obtained from a sample, it is converted to complementary DNA (cDNA) in a hybridization reaction. Conversion of the mRNA to cDNA can be performed with oligonucleotides or primers comprising sequence that is complementary to a portion of a specific mRNA. Conversion of the mRNA to cDNA can be performed with oligonucleotides or primers comprising random sequence. Conversion of the mRNA to cDNA can be performed with oligonucleotides or primers comprising sequence that is complementary to the poly(A) tail of an mRNA. cDNA does not exist in vivo and therefore is a non-natural molecule. In a further embodiment, the cDNA is then amplified, for example, by the polymerase chain reaction (PCR) or other amplification method known to those of ordinary skill in the art. PCR can be performed with the forward and/or reverse primers comprising sequence complementary to at least a portion of a classifier gene provided herein, such as the classifier biomarkers in Table 1 or Table 2. The product of this amplification reaction, i.e., amplified cDNA is necessarily a non-natural product. As mentioned above, cDNA is a non-natural molecule. Second, in the case of PCR, the amplification process serves to create hundreds of millions of cDNA copies for every individual cDNA molecule of starting material. The number of copies generated is far removed from the number of copies of mRNA that are present in vivo.

In one embodiment, cDNA is amplified with primers that introduce an additional DNA sequence (adapter sequence) onto the fragments (with the use of adapter-specific primers). The adaptor sequence can be a tail, wherein the tail sequence is not complementary to the cDNA. For example, the forward and/or reverse primers comprising sequence complementary to at least a portion of a classifier gene provided herein, such as the classifier biomarkers from Table 1 or Table 2 can comprise tail sequence. Amplification therefore serves to create non-natural double stranded molecules from the non-natural single stranded cDNA, by introducing barcode, adapter and/or reporter sequences onto the already non-natural cDNA. In one embodiment, during amplification with the adapter-specific primers, a detectable label, e.g., a fluorophore, is added to single strand cDNA molecules. Amplification therefore also serves to create DNA complexes that do not occur in nature, at least because (i) cDNA does not exist in vivo, (ii) adapter sequences are added to the ends of cDNA molecules to make DNA sequences that do not exist in vivo, (iii) the error rate associated with amplification further creates DNA sequences that do not exist in vivo, (iv) the disparate structure of the cDNA molecules as compared to what exists in nature, and (v) the chemical addition of a detectable label to the cDNA molecules.

In one embodiment, the synthesized cDNA (for example, amplified cDNA) is immobilized on a solid surface via hybridization with a probe, e.g., via a microarray. In another embodiment, cDNA products are detected via real-time polymerase chain reaction (PCR) via the introduction of fluorescent probes that hybridize with the cDNA products. For example, in one embodiment, biomarker detection is assessed by quantitative fluorogenic RT-PCR (e.g., with TaqMan® probes). For PCR analysis, well known methods are available in the art for the determination of primer sequences for use in the analysis.

Biomarkers provided herein in one embodiment, are detected via a hybridization reaction that employs a capture probe and/or a reporter probe. For example, the hybridization probe is a probe derivatized to a solid surface such as a bead, glass or silicon substrate. In another embodiment, the capture probe is present in solution and mixed with the patient's sample, followed by attachment of the hybridization product to a surface, e.g., via a biotin-avidin interaction (e.g., where biotin is a part of the capture probe and avidin is on the surface). The hybridization assay, in one embodiment, employs both a capture probe and a reporter probe. The reporter probe can hybridize to either the capture probe or the biomarker nucleic acid. Reporter probes e.g., are then counted and detected to determine the level of biomarker(s) in the sample. The capture and/or reporter probe, in one embodiment contain a detectable label, and/or a group that allows functionalization to a surface.

For example, the nCounter gene analysis system (see, e.g., Geiss et al. (2008) Nat. Biotechnol. 26, pp. 317-325, incorporated by reference in its entirety for all purposes, is amenable for use with the methods provided herein.

Hybridization assays described in U.S. Pat. Nos. 7,473,767 and 8,492,094, the disclosures of which are incorporated by reference in their entireties for all purposes, are amenable for use with the methods provided herein, i.e., to detect the biomarkers and biomarker combinations described herein.

Biomarker levels may be monitored using a membrane blot (such as used in hybridization analysis such as Northern, Southern, dot, and the like), or microwells, sample tubes, gels, beads, or fibers (or any solid support comprising bound nucleic acids). See, for example, U.S. Pat. Nos. 5,770,722, 5,874,219, 5,744,305, 5,677,195 and 5,445,934, each incorporated by reference in their entireties.

In one embodiment, microarrays are used to detect biomarker levels. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning hybridization intensities for each probe on the array are determined and converted to a quantitative value representing relative gene expression levels. See, for example, U.S. Pat. Nos. 6,040,138, 5,800,992 and 6,020,135, 6,033,860, and 6,344,316, each incorporated by reference in their entireties. High-density oligonucleotide arrays are particularly useful for determining the gene expression profile for a large number of RNAs in a sample.

Techniques for the synthesis of these arrays using mechanical synthesis methods are described in, for example, U.S. Pat. No. 5,384,261. Although a planar array surface is generally used, the array can be fabricated on a surface of virtually any shape or even a multiplicity of surfaces. Arrays can be nucleic acids (or peptides) on beads, gels, polymeric surfaces, fibers (such as fiber optics), glass, or any other appropriate substrate. See, for example, U.S. Pat. Nos. 5,770,358, 5,789,162, 5,708,153, 6,040,193 and 5,800,992, each incorporated by reference in their entireties. Arrays can be packaged in such a manner as to allow for diagnostics or other manipulation of an all-inclusive device. See, for example, U.S. Pat. Nos. 5,856,174 and 5,922,591, each incorporated by reference in their entireties.

Serial analysis of gene expression (SAGE) in one embodiment is employed in the methods described herein. SAGE is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. See, Velculescu et al. Science 270:484-87, 1995; Cell 88:243-51, 1997, incorporated by reference in its entirety.

An additional method of biomarker level analysis at the nucleic acid level is the use of a sequencing method, for example, RNAseq, next generation sequencing, and massively parallel signature sequencing (MPSS), as described by Brenner et al. (Nat. Biotech. 18:630-34, 2000, incorporated by reference in its entirety). This is a sequencing approach that combines non-gel-based signature sequencing with in vitro cloning of millions of templates on separate 5 μm diameter microbeads. First, a microbead library of DNA templates is constructed by in vitro cloning. This is followed by the assembly of a planar array of the template-containing microbeads in a flow cell at a high density (typically greater than 3.0×10⁶ microbeads/cm²). The free ends of the cloned templates on each microbead are analyzed simultaneously, using a fluorescence-based signature sequencing method that does not require DNA fragment separation. This method has been shown to simultaneously and accurately provide, in a single operation, hundreds of thousands of gene signature sequences from a yeast cDNA library.

Another method of biomarker level analysis at the nucleic acid level is the use of an amplification method such as, for example, RT-PCR or quantitative RT-PCR (qRT-PCR). Methods for determining the level of biomarker mRNA in a sample may involve the process of nucleic acid amplification, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. Numerous different PCR or qRT-PCR protocols are known in the art and can be directly applied or adapted for use using the presently described compositions for the detection and/or quantification of expression of discriminative genes in a sample. See, for example, Fan et al. (2004) Genome Res. 14:878-885, herein incorporated by reference. Generally, in PCR, a target polynucleotide sequence is amplified by reaction with at least one oligonucleotide primer or pair of oligonucleotide primers. The primer(s) hybridize to a complementary region of the target nucleic acid and a DNA polymerase extends the primer(s) to amplify the target sequence. Under conditions sufficient to provide polymerase-based nucleic acid amplification products, a nucleic acid fragment of one size dominates the reaction products (the target polynucleotide sequence which is the amplification product). The amplification cycle is repeated to increase the concentration of the single target polynucleotide sequence. The reaction can be performed in any thermocycler commonly used for PCR.

Quantitative RT-PCR (qRT-PCR) (also referred as real-time RT-PCR) is preferred under some circumstances because it provides not only a quantitative measurement, but also reduced time and contamination. As used herein, “quantitative PCR” (or “real time qRT-PCR”) refers to the direct monitoring of the progress of a PCR amplification as it is occurring without the need for repeated sampling of the reaction products. In quantitative PCR, the reaction products may be monitored via a signaling mechanism (e.g., fluorescence) as they are generated and are tracked after the signal rises above a background level but before the reaction reaches a plateau. The number of cycles required to achieve a detectable or “threshold” level of fluorescence varies directly with the concentration of amplifiable targets at the beginning of the PCR process, enabling a measure of signal intensity to provide a measure of the amount of target nucleic acid in a sample in real time. A DNA binding dye (e.g., SYBR green) or a labeled probe can be used to detect the extension product generated by PCR amplification. Any probe format utilizing a labeled probe comprising the sequences of the invention may be used.

Immunohistochemistry methods are also suitable for detecting the levels of the biomarkers of the present invention. Samples can be frozen for later preparation or immediately placed in a fixative solution. Tissue samples can be fixed by treatment with a reagent, such as formalin, gluteraldehyde, methanol, or the like and embedded in paraffin. Methods for preparing slides for immunohistochemical analysis from formalin-fixed, paraffin-embedded tissue samples are well known in the art.

In one embodiment, the levels of the biomarkers provided herein, such as the classifier biomarkers of Table 1 or Table 2 (or subsets thereof as provided herein), are normalized against the expression levels of all RNA transcripts or their non-natural cDNA expression products, or protein products in the sample, or of a reference set of RNA transcripts or a reference set of their non-natural cDNA expression products, or a reference set of their protein products in the sample.

In one embodiment, an AF-PRS can be evaluated using levels of protein expression of one or more of the classifier genes provided herein, such as the classifier biomarkers listed in Table 1. In one embodiment, proliferation can be evaluated using levels of protein expression of one or more of the classifier genes provided herein, such as the classifier biomarkers listed in Table 2. The level of protein expression can be measured using an immunological detection method. Immunological detection methods which can be used herein include, but are not limited to, competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, and the like. Such assays are routine and well known in the art (see, e.g., Ausubel et al, eds, 1994, Current Protocols in Molecular Biology, Vol. I, John Wiley & Sons, Inc., New York, which is incorporated by reference herein in its entirety).

In one embodiment, antibodies specific for biomarker proteins are utilized to detect the expression of a biomarker protein in a body sample. The method comprises obtaining a body sample from a patient or a subject, contacting the body sample with at least one antibody directed to a biomarker that is selectively expressed in lung cancer cells, and detecting antibody binding to determine if the biomarker is expressed in the patient sample. A preferred aspect of the present invention provides an immunocytochemistry technique for diagnosing lung cancer subtypes. One of skill in the art will recognize that the immunocytochemistry method described herein below may be performed manually or in an automated fashion.

As provided throughout, the methods set forth herein provide a method for determining the AF-PRS or proliferation of a subject. Once the biomarker levels are determined, for example by measuring non-natural cDNA biomarker levels or non-natural mRNA-cDNA biomarker complexes, the biomarker levels are compared to reference values or a reference sample, for example with the use of statistical methods or direct comparison of detected levels, to make a determination of the AF-PRS or proliferation or proliferation score. Based on the comparison, the patient's sample is classified as being AF-PRS (+) or (−) or possessing proliferation.

In one embodiment, expression level values of the at least one classifier biomarkers provided herein, such as the classifier biomarkers of Table 1 are compared to reference expression level value(s) from at least one sample training set, wherein the at least one sample training set comprises expression level values from a reference sample(s). In a further embodiment, the at least one sample training set comprises expression level values of the at least one classifier biomarkers provided herein, such as the classifier biomarkers of Table 1 from a terminal respiratory unit (bronchioid) sample or non-bronchioid sample (proximal inflammatory (squamoid) alone, proximal proliferative (magnoid) alone, or both proximal inflammatory (squamoid) and proximal proliferative (magnoid)) or a combination thereof.

In a separate embodiment, hybridization values of the at least one classifier biomarkers provided herein, such as the classifier biomarkers of Table 1 are compared to reference hybridization value(s) from at least one sample training set, wherein the at least one sample training set comprises hybridization values from a reference sample(s). In a further embodiment, the at least one sample training set comprises hybridization values of the at least one classifier biomarkers provided herein, such as the classifier biomarkers of Table 1 from a terminal respiratory unit (bronchioid) sample or non-bronchioid sample (proximal inflammatory (squamoid) alone, proximal proliferative (magnoid) alone, or both proximal inflammatory (squamoid) and proximal proliferative (magnoid)) or a combination thereof. Methods for comparing detected levels of biomarkers to reference values and/or reference samples are provided herein. Based on this comparison, in one embodiment a correlation between the biomarker levels obtained from the subject's sample and the reference values is obtained. An assessment of the AF-PRS is then made.

In one embodiment, expression level values of the at least one classifier biomarkers provided herein, such as the classifier biomarkers of Table 2 are compared to reference expression level value(s) from at least one sample training set, wherein the at least one sample training set comprises expression level values from a reference sample(s). In a further embodiment, the at least one sample training set comprises expression level values of the at least one classifier biomarkers provided herein, such as the classifier biomarkers of Table 2 from a proliferative sample or non-proliferative sample or a combination thereof.

In a separate embodiment, hybridization values of the at least one classifier biomarkers provided herein, such as the classifier biomarkers of Table 2 are compared to reference hybridization value(s) from at least one sample training set, wherein the at least one sample training set comprises hybridization values from a reference sample(s). In a further embodiment, the at least one sample training set comprises hybridization values of the at least one classifier biomarkers provided herein, such as the classifier biomarkers of Table 2 from a proliferative sample or non-proliferative sample or a combination thereof. Methods for comparing detected levels of biomarkers to reference values and/or reference samples are provided herein. Based on this comparison, in one embodiment a correlation between the biomarker levels obtained from the subject's sample and the reference values is obtained. An assessment of proliferation is then made.

### Sample Types

In one embodiment, the sample used in any method provided herein is obtained from an individual and comprises formalin-fixed paraffin-embedded (FFPE) tissue. However, other tissue and sample types are amenable for use in any of the methods provided herein. In one embodiment, the other tissue and sample types can be fresh frozen tissue, wash fluids or cell pellets, or the like. In one embodiment, the sample can be a bodily fluid obtained from the individual. The bodily fluid can be blood or fractions thereof (e.g., serum, plasma), urine, sputum, saliva or cerebrospinal fluid (CSF). A biomarker nucleic acid (e.g., DNA or RNA) as provided herein can be extracted from a cell or can be cell free or extracted from an extracellular vesicular entity such as an exosome or microvesicle. The sample can contain cellular as well as extracellular sources of nucleic acid for use in the methods provided herein. The methods provided herein, including the RT-PCR methods, are sensitive, precise and have multi-analyte capability for use with paraffin embedded samples. See, for example, Cronin et al. (2004) Am. J Pathol. 164(1):35-42, herein incorporated by reference.

Formalin fixation and tissue embedding in paraffin wax is a universal approach for tissue processing prior to light microscopic evaluation. A major advantage afforded by formalin-fixed paraffin-embedded (FFPE) specimens is the preservation of cellular and architectural morphologic detail in tissue sections. (Fox et al. (1985) J Histochem Cytochem 33:845-853). The standard buffered formalin fixative in which biopsy specimens are processed is typically an aqueous solution containing 37% formaldehyde and 10-15% methyl alcohol. Formaldehyde is a highly reactive dipolar compound that results in the formation of protein-nucleic acid and protein-protein crosslinks in vitro (Clark et al. (1986) J Histochem Cytochem 34:1509-1512; McGhee and von Hippel (1975) Biochemistry 14:1281-1296, each incorporated by reference herein).

Methods are known in the art for the isolation of RNA from FFPE tissue. In one embodiment, total RNA can be isolated from FFPE tissues as described by Bibikova et al. (2004) American Journal of Pathology 165:1799-1807, herein incorporated by reference. Likewise, the High Pure RNA Paraffin Kit (Roche) can be used. Paraffin is removed by xylene extraction followed by ethanol wash. RNA can be isolated from sectioned tissue blocks using the MasterPure Purification kit (Epicenter, Madison, Wis.); a DNase I treatment step is included. RNA can be extracted from frozen samples using Trizol reagent according to the supplier's instructions (Invitrogen Life Technologies, Carlsbad, Calif.). Samples with measurable residual genomic DNA can be resubjected to DNaseI treatment and assayed for DNA contamination. All purification, DNase treatment, and other steps can be performed according to the manufacturer's protocol. After total RNA isolation, samples can be stored at −80° C. until use.

General methods for mRNA extraction are well known in the art and are disclosed in standard textbooks of molecular biology, including Ausubel et al., ed., Current Protocols in Molecular Biology, John Wiley & Sons, New York 1987-1999. Methods for RNA extraction from paraffin embedded tissues are disclosed, for example, in Rupp and Locker (Lab Invest. 56:A67, 1987) and De Andres et al. (Biotechniques 18:42-44, 1995). In particular, RNA isolation can be performed using a purification kit, a buffer set and protease from commercial manufacturers, such as Qiagen (Valencia, Calif.), according to the manufacturer's instructions. For example, total RNA from cells in culture can be isolated using Qiagen RNeasy mini-columns. Other commercially available RNA isolation kits include MasterPure™. Complete DNA and RNA Purification Kit (Epicentre, Madison, Wis.) and Paraffin Block RNA Isolation Kit (Ambion, Austin, Tex.). Total RNA from tissue samples can be isolated, for example, using RNA Stat-60 (Tel-Test, Friendswood, Tex.). RNA prepared from a tumor can be isolated, for example, by cesium chloride density gradient centrifugation. Additionally, large numbers of tissue samples can readily be processed using techniques well known to those of skill in the art, such as, for example, the single-step RNA isolation process of Chomczynski (U.S. Pat. No. 4,843,155, incorporated by reference in its entirety for all purposes).

In one embodiment, a sample for use in any of the methods provided herein comprises cells harvested from a tissue sample, for example, a tumor sample. The tumor sample can be a cancerous tumor. The cancerous tumor can be any type of cancer known in the art and/or provided herein. Cells can be harvested from a biological sample using standard techniques known in the art. For example, in one embodiment, cells are harvested by centrifuging a cell sample and resuspending the pelleted cells. The cells can be resuspended in a buffered solution such as phosphate-buffered saline (PBS). After centrifuging the cell suspension to obtain a cell pellet, the cells can be lysed to extract nucleic acid, e.g, messenger RNA. All samples obtained from a subject, including those subjected to any sort of further processing, are considered to be obtained from the subject.

The sample, in one embodiment, is further processed before the detection of the biomarker levels of the combination of biomarkers set forth herein. For example, mRNA in a cell or tissue sample can be separated from other components of the sample. The sample can be concentrated and/or purified to isolate mRNA in its non-natural state, as the mRNA is not in its natural environment. For example, studies have indicated that the higher order structure of mRNA in vivo differs from the in vitro structure of the same sequence (see, e.g., Rouskin et al. (2014). Nature 505, pp. 701-705, incorporated herein in its entirety for all purposes).

mRNA from the sample in one embodiment, is hybridized to a synthetic DNA probe, which in some embodiments, includes a detection moiety (e.g., detectable label, capture sequence, barcode reporting sequence). Accordingly, in these embodiments, a non-natural mRNA-cDNA complex is ultimately made and used for detection of the biomarker. In another embodiment, mRNA from the sample is directly labeled with a detectable label, e.g., a fluorophore. In a further embodiment, the non-natural labeled-mRNA molecule is hybridized to a cDNA probe and the complex is detected.

In one embodiment, once the mRNA is obtained from a sample, it is converted to complementary DNA (cDNA) in a hybridization reaction or is used in a hybridization reaction together with one or more cDNA probes. cDNA does not exist in vivo and therefore is a non-natural molecule. Furthermore, cDNA-mRNA hybrids are synthetic and do not exist in vivo. Besides cDNA not existing in vivo, cDNA is necessarily different than mRNA, as it includes deoxyribonucleic acid and not ribonucleic acid. The cDNA is then amplified, for example, by the polymerase chain reaction (PCR) or other amplification method known to those of ordinary skill in the art. For example, other amplification methods that may be employed include the ligase chain reaction (LCR) (Wu and Wallace, Genomics, 4:560 (1989), Landegren et al., Science, 241:1077 (1988), incorporated by reference in its entirety for all purposes, transcription amplification (Kwoh et al., Proc. Natl. Acad. Sci. USA, 86:1173 (1989), incorporated by reference in its entirety for all purposes), self-sustained sequence replication (Guatelli et al., Proc. Nat. Acad. Sci. USA, 87:1874 (1990), incorporated by reference in its entirety for all purposes), incorporated by reference in its entirety for all purposes, and nucleic acid based sequence amplification (NASBA). Guidelines for selecting primers for PCR amplification are known to those of ordinary skill in the art. See, e.g., McPherson et al., PCR Basics: From Background to Bench, Springer-Verlag, 2000, incorporated by reference in its entirety for all purposes. The product of this amplification reaction, i.e., amplified cDNA is also necessarily a non-natural product. First, as mentioned above, cDNA is a non-natural molecule. Second, in the case of PCR, the amplification process serves to create hundreds of millions of cDNA copies for every individual cDNA molecule of starting material. The numbers of copies generated are far removed from the number of copies of mRNA that are present in vivo.

In one embodiment, cDNA is amplified with primers that introduce an additional DNA sequence (e.g., adapter, reporter, capture sequence or moiety, barcode) onto the fragments (e.g., with the use of adapter-specific primers), or mRNA or cDNA biomarker sequences are hybridized directly to a cDNA probe comprising the additional sequence (e.g., adapter, reporter, capture sequence or moiety, barcode). Amplification and/or hybridization of mRNA to a cDNA probe therefore serves to create non-natural double stranded molecules from the non-natural single stranded cDNA, or the mRNA, by introducing additional sequences and forming non-natural hybrids. Further, as known to those of ordinary skill in the art, amplification procedures have error rates associated with them. Therefore, amplification introduces further modifications into the cDNA molecules. In one embodiment, during amplification with the adapter-specific primers, a detectable label, e.g., a fluorophore, is added to single strand cDNA molecules. Amplification therefore also serves to create DNA complexes that do not occur in nature, at least because (i) cDNA does not exist in vivo, (i) adapter sequences are added to the ends of cDNA molecules to make DNA sequences that do not exist in vivo, (ii) the error rate associated with amplification further creates DNA sequences that do not exist in vivo, (iii) the disparate structure of the cDNA molecules as compared to what exists in nature, and (iv) the chemical addition of a detectable label to the cDNA molecules.

In some embodiments, the expression of a biomarker of interest (e.g., one or a plurality of biomarkers from Table 1 or Table 2) is detected at the nucleic acid level via detection of non-natural cDNA molecules.

### Types of Cancer

Further to any of the embodiments provided herein, the sample obtained from a subject subjected any of the methods provided herein can be a tumor sample. The tumor sample can be a cancerous tumor. The cancer can include, but is not limited to, carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. Examples of a cancer also include, but are not limited to, a lung cancer (e.g., a non-small cell lung cancer (NSCLC) such as lung adenocarcinoma (LUAD) or lung squamous cell carcinoma (LUSC)), a kidney cancer (e.g., a kidney urothelial carcinoma or RCC), a bladder cancer (e.g., a bladder urothelial (transitional cell) carcinoma (e.g., locally advanced or metastatic urothelial cancer, including 1L or 2L+ locally advanced or metastatic urothelial carcinoma), a breast cancer, a colorectal cancer (e.g., a colon adenocarcinoma), an ovarian cancer, a pancreatic cancer (e.g., pancreatic adenocarcinoma or PAAD), a gastric carcinoma, an esophageal cancer, a mesothelioma, a melanoma (e.g., a skin melanoma), a head and neck cancer (e.g., a head and neck squamous cell carcinoma (HNSCC)), a thyroid cancer, a sarcoma (e.g., a soft-tissue sarcoma, a fibrosarcoma, a myxosarcoma, a liposarcoma, an osteogenic sarcoma, an osteosarcoma, a chondrosarcoma, an angiosarcoma, an endotheliosarcoma, a lymphangiosarcoma, a lymphangioendotheliosarcoma, a leiomyosarcoma, or a rhabdomyosarcoma), a prostate cancer, a glioblastoma, a cervical cancer, a thymic carcinoma, a leukemia (e.g., an acute lymphocytic leukemia (ALL), an acute myelocytic leukemia (AML), a chronic myelocytic leukemia (CML), a chronic eosinophilic leukemia, or a chronic lymphocytic leukemia (CLL)), a lymphoma (e.g., a Hodgkin lymphoma or a non-Hodgkin lymphoma (NHL)), a myeloma (e.g., a multiple myeloma (MM)), a mycosis fungoides, a Merkel cell cancer, a hematologic malignancy, a cancer of hematological tissues, a B cell cancer, a bronchus cancer, a stomach cancer, a brain or central nervous system cancer, a peripheral nervous system cancer, a uterine or endometrial cancer, a cancer of the oral cavity or pharynx, a liver cancer, a testicular cancer, a biliary tract cancer, a small bowel or appendix cancer, a salivary gland cancer, an adrenal gland cancer, an adenocarcinoma, an inflammatory myofibroblastic tumor, a gastrointestinal stromal tumor (GIST), a colon cancer, a myelodysplastic syndrome (MDS), a myeloproliferative disorder (MPD), a polycythemia Vera, a chordoma, a synovioma, an Ewing's tumor, a squamous cell carcinoma, a basal cell carcinoma, an adenocarcinoma, a sweat gland carcinoma, a sebaceous gland carcinoma, a papillary carcinoma, a papillary adenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilms' tumor, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodendroglioma, a meningioma, a neuroblastoma, a retinoblastoma, a follicular lymphoma, a diffuse large B-cell lymphoma, a mantle cell lymphoma, a hepatocellular carcinoma, a thyroid cancer, a small cell cancer, an essential thrombocythemia, an agnogenic myeloid metaplasia, a hypereosinophilic syndrome, a systemic mastocytosis, a familiar hypereosinophilia, a neuroendocrine cancer, or a carcinoid tumor.

In some cases, the cancer that the subject from which a sample is obtained is suffering or suspected of suffering from is selected from a cervical kidney renal papillary cell carcinoma (KIRP); breast invasive carcinoma (BRCA); thyroid ancer (THCA); bladder carcinoma (BLCA); prostate adenocarcinoma (PRAD); kidney chromophobe (KICH); cervical squamous cell carcinoma and endocervical adenocarcinoma (CESC); kidney renal clear cell carcinoma (KIRC); liver hepatocellular carcinoma (LIHC); low grade glioma (LGG); sarcoma (SARC); lung adenocarcinoma (LUAD); colon adenocarcinoma (COAD); head-neck squamous cell carcinoma (HNSC); uterine corpus endometrial carcinoma (UCEC); glioblastoma multiforme (GBM); esophageal carcinoma (ESCA); stomach adenocarcinoma (STAD); ovarian cancer (OV); rectum adenocarcinoma (READ), adrenocortical carcinoma (ACC), mesothelioma MESO) or lung squamous cell carcinoma (LUSC), an esophageal cancer, a mesothelioma, a melanoma, a head and neck cancer, a thyroid cancer, a sarcoma, a prostate cancer, a glioblastoma, a cervical cancer, a thymic carcinoma, a leukemia, a lymphoma, a myeloma, a mycosis fungoides, a merkel cell cancer, an endometrial cancer. In some cases, the cancer is LUAD, LGG, LIHC, KIRC, KICH, MESO, ACC or KIRP.

### Statistical Methods

Various statistical methods can be used to aid in the comparison of the biomarker levels obtained from the patient and reference biomarker levels, for example, from at least one sample training set.

In one embodiment, a supervised pattern recognition method is employed. Examples of supervised pattern recognition methods can include, but are not limited to, the nearest centroid methods (Dabney (2005) Bioinformatics 21(22):4148-4154 and Tibshirani et al. (2002) Proc. Natl. Acad. Sci. USA 99(10):6576-6572); soft independent modeling of class analysis (SIMCA) (see, for example, Wold, 1976); partial least squares analysis (PLS) (see, for example, Wold, 1966; Joreskog, 1982; Frank, 1984; Bro, R., 1997); linear descriminant analysis (LDA) (see, for example, Nillson, 1965); K-nearest neighbour analysis (KNN) (sec, for example, Brown et al., 1996); artificial neural networks (ANN) (see, for example, Wasserman, 1989; Anker et al., 1992; Hare, 1994); probabilistic neural networks (PNNs) (see, for example, Parzen, 1962; Bishop, 1995; Speckt, 1990; Broomhead et al., 1988; Patterson, 1996); rule induction (RI) (see, for example, Quinlan, 1986); and, Bayesian methods (see, for example, Bretthorst, 1990a, 1990b, 1988). In one embodiment, the classifier for identifying tumor subtypes based on gene expression data is the centroid based method described in Mullins et al. (2007) Clin Chem. 53(7):1273-9, each of which is herein incorporated by reference in its entirety. In another embodiment, the classifier for identifying AF-PRS based on gene expression data is used in a nearest centroid based method as described in Dabney (2005) Bioinformatics 21(22):4148-4154, which is incorporated herein by reference in its entirety. The nearest centroid based method can be performed using CLaNC software as described in Dabney A R. ClaNC: Point-and-click software for classifying microarrays to nearest centroids. Bioinformatics. 2006; 22: 122-123 or equivalents or derivatives thereof.

In other embodiments, an unsupervised training approach is employed, and therefore, no training set is used.

Referring to sample training sets for supervised learning approaches again, in some embodiments, a sample training set(s) can include expression data of a plurality or all of the classifier biomarkers (e.g., all the classifier biomarkers of Table 1 or Table 2) from an adenocarcinoma sample. The plurality of classifier biomarkers can comprise at least two classifier biomarkers, at least 8 classifier biomarkers, at least 16 classifier biomarkers, at least 24 classifier biomarkers, at least 32 classifier biomarkers, at least 40 classifier biomarkers, or at least 48 classifier biomarkers of Table 1. The plurality of classifier biomarkers can comprise at least two classifier biomarkers, at least 2 classifier biomarkers, at least 4 classifier biomarkers, at least 6 classifier biomarkers, at least 8 classifier biomarkers, at least 10 classifier biomarkers, at least 12 classifier biomarkers, at least 14 classifier biomarkers, at least 16 classifier biomarkers, at least 18 classifier biomarkers, at least 20 classifier biomarkers, at least 22 classifier biomarkers, at least 24 classifier biomarkers, or at least 26 classifier biomarkers of Table 2. In some embodiments, the sample training set(s) are normalized to remove sample-to-sample variation.

In some embodiments, comparing can include applying a statistical algorithm, such as, for example, any suitable multivariate statistical analysis model, which can be parametric or non-parametric. In some embodiments, applying the statistical algorithm can include determining a correlation between the expression data obtained from the human lung tissue sample and the expression data from the adenocarcinoma training set(s). In some embodiments, cross-validation is performed, such as (for example), leave-one-out cross-validation (LOOCV). In some embodiments, integrative correlation is performed. In some embodiments, a Spearman correlation is performed. In some embodiments, a centroid based method is employed for the statistical algorithm. The centroids can be constructed using any method known in the art for generating centroids such as, for example, those found in Mullins et al. (2007) Clin Chem. 53(7):1273-9 or the nearest centroid method found in Dabney (2005) Bioinformatics 21(22):4148-4154, which is herein incorporated by reference in its entirety. In one embodiment, a correlation analysis is performed on the expression data obtained from the sample obtained from a subject suffering or suspected of suffering from a cancer and the centroid(s) constructed on the expression data from the training set(s). The correlation analysis can be a Spearman correlation or a Pearson correlation. In one embodiment, a distance measure analysis (e.g., Euclidean distance) is performed on the expression data obtained from the sample and the centroid(s) constructed on the expression data from the training set(s).

Results of the gene expression performed on a sample from a subject (test sample) may be compared to a biological sample(s) or data derived from a reference biological sample(s). In some embodiments for assessing the AF-PRS, a reference sample or reference gene expression data is obtained or derived from an individual known to have a particular molecular subtype of adenocarcinoma, i.e., bronchioid (terminal respiratory unit) or non-bronchioid (squamoid (proximal inflammatory) and/or magnoid (proximal proliferative)). In some embodiments for assessing proliferation, a reference sample or reference gene expression data is obtained or derived from an individual known to be proliferative. In one embodiment, the gene expression levels or profile for the at least one classifier biomarker provided herein (e.g., Table 1 or 2) measured or detected in the test sample may be compared to centroids constructed from the gene expression performed on the reference sample. The centroids can be constructed using any of the methods provided herein such as, for example, using the ClaNC software described in Dabney A R. ClaNC: Point-and-click software for classifying microarrays to nearest centroids. Bioinformatics. 2006; 22: 122-123 or equivalents or derivatives related thereto. Classification or determination of the subtype of the test sample can then be ascertained by determining the nearest centroid from the reference or normal sample to which the expression levels or profile from said test sample is nearest based on a distance measure or correlation. The distance measure can be a Euclidean distance. In embodiments related to determining an AF-PRS, the bronchioid and/or non-bronchioid (i.e., magnoid and/or squamoid) centroids can be the centroids found in Table 4.

The reference sample may be assayed at the same time, or at a different time from the test sample. Alternatively, the biomarker level information from a reference sample may be stored in a database or other means for access at a later date.

The biomarker level results of an assay on the test sample may be compared to the results of the same assay on a reference sample. In some cases, the results of the assay on the reference sample are from a database, or a reference value(s). In some cases, the results of the assay on the reference sample are a known or generally accepted value or range of values by those skilled in the art. In some cases, the comparison is qualitative. In other cases, the comparison is quantitative. In some cases, qualitative or quantitative comparisons may involve but are not limited to one or more of the following: comparing fluorescence values, spot intensities, absorbance values, chemiluminescent signals, histograms, critical threshold values, statistical significance values, expression levels of the genes described herein, mRNA copy numbers.

In one embodiment, an odds ratio (OR) is calculated for each biomarker level panel measurement. Here, the OR is a measure of association between the measured biomarker values for the patient and an outcome, e.g., anti-folate predictive response signature or determination of proliferation status. For example, see, J Can. Acad. Child Adolesc. Psychiatry 2010; 19(3): 227-229, which is incorporated by reference in its entirety for all purposes.

In one embodiment, a specified statistical confidence level may be determined in order to provide a confidence level regarding the anti-folate predictive response signature or proliferation status. For example, it may be determined that a confidence level of greater than 90% may be a useful predictor of the anti-folate predictive response signature or proliferation status. In other embodiments, more or less stringent confidence levels may be chosen. For example, a confidence level of about or at least about 50%, 60%, 70%, 75%, 80%, 85%, 90%, 95%, 97.5%, 99%, 99.5%, or 99.9% may be chosen. The confidence level provided may in some cases be related to the quality of the sample, the quality of the data, the quality of the analysis, the specific methods used, and/or the number of gene expression values (i.e., the number of genes) analyzed. The specified confidence level for providing the likelihood of response may be chosen on the basis of the expected number of false positives or false negatives. Methods for choosing parameters for achieving a specified confidence level or for identifying markers with diagnostic power include but are not limited to Receiver Operating Characteristic (ROC) curve analysis, binormal ROC, principal component analysis, odds ratio analysis, partial least squares analysis, singular value decomposition, least absolute shrinkage and selection operator analysis, least angle regression, and the threshold gradient directed regularization method.

Determining the anti-folate predictive response signature or proliferation status in some cases can be improved through the application of algorithms designed to normalize and or improve the reliability of the gene expression data. In some embodiments of the present invention, the data analysis utilizes a computer or other device, machine or apparatus for application of the various algorithms described herein due to the large number of individual data points that are processed. A “machine learning algorithm” refers to a computational-based prediction methodology, also known to persons skilled in the art as a “classifier,” employed for characterizing a gene expression profile or profiles, e.g., to determine the anti-folate predictive response signature or proliferation status. The biomarker levels, determined by, e.g., microarray-based hybridization assays, sequencing assays, NanoString assays, etc., are in one embodiment subjected to the algorithm in order to classify the profile. In embodiment related to assessing an anti-folate predictive response signature, supervised learning generally involves “training” a classifier to recognize the distinctions among anti-folate predictive response signatures such as bronchioid (terminal respiratory unit) positive or non-bronchioid positive (i.e., squamoid (proximal inflammatory) positive and/or magnoid (proximal proliferative) positive), and then “testing” the accuracy of the classifier on an independent test set. Therefore, for new, unknown samples the classifier can be used to predict, for example, the class (e.g., bronchioid vs. non-bronchioid) in which the samples belong. In embodiment related to assessing proliferation status, supervised learning generally involves “training” a classifier to recognize the distinctions among proliferation statuses or scores such as proliferative or non-proliferative and then “testing” the accuracy of the classifier on an independent test set. Therefore, for new, unknown samples the classifier can be used to predict, for example, the class (e.g., proliferative vs. non-proliferative) in which the samples belong.

In some embodiments, a robust multi-array average (RMA) method may be used to normalize raw data. The RMA method begins by computing background-corrected intensities for each matched cell on a number of microarrays. In one embodiment, the background corrected values are restricted to positive values as described by Irizarry et al. (2003). Biostatistics April 4 (2): 249-64, incorporated by reference in its entirety for all purposes. After background correction, the base-2 logarithm of each background corrected matched-cell intensity is then obtained. The background corrected, log-transformed, matched intensity on each microarray is then normalized using the quantile normalization method in which for each input array and each probe value, the array percentile probe value is replaced with the average of all array percentile points, this method is more completely described by Bolstad et al. Bioinformatics 2003, incorporated by reference in its entirety. Following quantile normalization, the normalized data may then be fit to a linear model to obtain an intensity measure for each probe on each microarray. Tukey's median polish algorithm (Tukey, J. W., Exploratory Data Analysis. 1977, incorporated by reference in its entirety for all purposes) may then be used to determine the log-scale intensity level for the normalized probe set data.

Various other software programs may be implemented. In certain methods, feature selection and model estimation may be performed by logistic regression with lasso penalty using glmnet (Friedman et al. (2010). Journal of statistical software 33(1): 1-22, incorporated by reference in its entirety). Raw reads may be aligned using TopHat (Trapnell et al. (2009). Bioinformatics 25(9): 1105-11, incorporated by reference in its entirety). In methods, top features (N ranging from 10 to 200) are used to train a linear support vector machine (SVM) (Suykens J A K, Vandewalle J. Least Squares Support Vector Machine Classifiers. Neural Processing Letters 1999; 9(3): 293-300, incorporated by reference in its entirety) using the e1071 library (Meyer D. Support vector machines: the interface to libsvm in package e1071. 2014, incorporated by reference in its entirety). Confidence intervals, in one embodiment, are computed using the pROC package (Robin X, Turck N, Hainard A, et al. pROC: an open-source package for R and S+ to analyze and compare ROC curves. BMC bioinformatics 2011; 12: 77, incorporated by reference in its entirety).

In addition, data may be filtered to remove data that may be considered suspect. In one embodiment, data derived from microarray probes that have fewer than about 4, 5, 6, 7 or 8 guanosine+cytosine nucleotides may be considered to be unreliable due to their aberrant hybridization propensity or secondary structure issues. Similarly, data deriving from microarray probes that have more than about 12, 13, 14, 15, 16, 17, 18, 19, 20, 21, or 22 guanosine+cytosine nucleotides may in one embodiment be considered unreliable due to their aberrant hybridization propensity or secondary structure issues.

In some embodiments of the present invention, data from probe-sets may be excluded from analysis if they are not identified at a detectable level (above background).

In some embodiments of the present disclosure, probe-sets that exhibit no, or low variance may be excluded from further analysis. Low-variance probe-sets are excluded from the analysis via a Chi-Square test. In one embodiment, a probe-set is considered to be low-variance if its transformed variance is to the left of the 99 percent confidence interval of the Chi-Squared distribution with (N-l) degrees of freedom. (N-l)*Probe-set Variance/(Gene Probe-set Variance). Chi-Sq(N-l) where N is the number of input CEL files, (N-l) is the degrees of freedom for the Chi-Squared distribution, and the “probe-set variance for the gene” is the average of probe-set variances across the gene. In some embodiments of the present invention, probe-sets for a given mRNA or group of mRNAs may be excluded from further analysis if they contain less than a minimum number of probes that pass through the previously described filter steps for GC content, reliability, variance and the like. For example, in some embodiments, probe-sets for a given gene or transcript cluster may be excluded from further analysis if they contain less than about 1, 2, 3, 4, 5, 6, 7, 8, 9, 10, 11, 12, 13, 14, 15, or less than about 20 probes.

Methods of biomarker level data analysis in one embodiment, further include the use of a feature selection algorithm as provided herein. In some embodiments of the present invention, feature selection is provided by use of the LIMMA software package (Smyth, G. K. (2005). Limma: linear models for microarray data. In: Bioinformatics and Computational Biology Solutions using R and Bioconductor, R. Gentleman, V. Carey, S. Dudoit, R. Irizarry, W. Huber (eds.), Springer, New York, pages 397-420, incorporated by reference in its entirety for all purposes).

Methods of biomarker level data analysis, in one embodiment, include the use of a pre-classifier algorithm. For example, an algorithm may use a specific molecular fingerprint to pre-classify the samples according to their composition and then apply a correction/normalization factor. This data/information may then be fed into a final classification algorithm which would incorporate that information to aid in the final diagnosis.

Methods of biomarker level data analysis, in one embodiment, further include the use of a classifier algorithm as provided herein. In one embodiment of the present invention, a diagonal linear discriminant analysis, k-nearest neighbor algorithm, support vector machine (SVM) algorithm, linear support vector machine, random forest algorithm, or a probabilistic model-based method or a combination thereof is provided for classification of microarray data. In some embodiments, identified markers that distinguish samples (e.g., of varying biomarker level profiles, and/or varying molecular anti-folate predictive response signatures (e.g., AF-PRS (+), AF-PRS (−)) are selected based on statistical significance of the difference in biomarker levels between classes of interest. In some embodiments, identified markers that distinguish samples (e.g., of varying biomarker level profiles, and/or varying molecular proliferation status (e.g., proliferation (+), proliferation (−)) are selected based on statistical significance of the difference in biomarker levels between classes of interest. In some cases, the statistical significance is adjusted by applying a Benjamin Hochberg or another correction for false discovery rate (FDR).

In some cases, the classifier algorithm may be supplemented with a meta-analysis approach such as that described by Fishel and Kaufman et al. 2007 Bioinformatics 23(13): 1599-606, incorporated by reference in its entirety for all purposes. In some cases, the classifier algorithm may be supplemented with a meta-analysis approach such as a repeatability analysis.

Methods for deriving and applying posterior probabilities to the analysis of biomarker level data are known in the art and have been described for example in Smyth, G. K. 2004 Stat. Appi. Genet. Mol. Biol. 3: Article 3, incorporated by reference in its entirety for all purposes. In some cases, the posterior probabilities may be used in the methods of the present invention to rank the markers provided by the classifier algorithm.

A statistical evaluation of the results of the biomarker level profiling may provide a quantitative value or values indicative of one or more of the following: anti-folate predictive response signature (e.g., bronchioid or non-bronchioid) or proliferation status (proliferative or non-proliferative); the likelihood of the success of a particular therapeutic intervention, e.g., anti-folate therapy, angiogenesis inhibitor therapy, chemotherapy, or immunotherapy. In one embodiment, the data is presented directly to the physician in its most useful form to guide patient care or is used to define patient populations in clinical trials or a patient population for a given medication. The results of the molecular profiling can be statistically evaluated using a number of methods known to the art including, but not limited to: the students T test, the two sided T test, Pearson rank sum analysis, hidden Markov model analysis, analysis of q-q plots, principal component analysis, one way ANOVA, two way ANOVA, LIMMA and the like.

In some cases, accuracy may be determined by tracking the subject over time to determine the accuracy of the original diagnosis. In other cases, accuracy may be established in a deterministic manner or using statistical methods. For example, receiver operator characteristic (ROC) analysis may be used to determine the optimal assay parameters to achieve a specific level of accuracy, specificity, positive predictive value, negative predictive value, and/or false discovery rate.

In some cases, the results of the biomarker level profiling assays, are entered into a database for access by representatives or agents of a molecular profiling business, the individual, a medical provider, or insurance provider. In some cases, assay results include sample classification, identification, or diagnosis by a representative, agent or consultant of the business, such as a medical professional. In other cases, a computer or algorithmic analysis of the data is provided automatically. In some cases, the molecular profiling business may bill the individual, insurance provider, medical provider, researcher, or government entity for one or more of the following: molecular profiling assays performed, consulting services, data analysis, reporting of results, or database access.

In some embodiments of the present invention, the results of the biomarker level profiling assays are presented as a report on a computer screen or as a paper record. In some embodiments, the report may include, but is not limited to, such information as one or more of the following: the levels of biomarkers (e.g., as reported by copy number or fluorescence intensity, etc.) as compared to the reference sample or reference value(s); the likelihood the subject will respond to a particular therapy, based on the biomarker level values and the anti-folate predictive response signature and/or proliferation status or score and proposed therapies.

In one embodiment, the results of the gene expression profiling may be classified into one or more of the following: bronchioid (terminal respiratory unit) positive, non-bronchioid positive (magnoid (proximal proliferative) positive and/or squamoid (proximal inflammatory) positive), bronchioid (terminal respiratory unit) negative, non-bronchioid negative (magnoid (proximal proliferative) negative and/or squamoid (proximal inflammatory) negative); likely to respond to anti-folate therapy, angiogenesis inhibitor, immunotherapy or chemotherapy; unlikely to respond to anti-folate therapy, angiogenesis inhibitor, immunotherapy or chemotherapy; or a combination thereof.

In one embodiment, the results of the gene expression profiling may be classified into one or more of the following: proliferation positive, non-proliferation positive, proliferation negative, non-proliferation negative; likely to respond to anti-folate therapy, angiogenesis inhibitor, immunotherapy or chemotherapy; unlikely to respond to anti-folate therapy, angiogenesis inhibitor, immunotherapy or chemotherapy; or a combination thereof.

In some embodiments of the present invention, results are classified using a trained algorithm. Trained algorithms of the present invention include algorithms that have been developed using a reference set of known gene expression values and/or normal samples, for example, samples from individuals diagnosed with a particular anti-folate predictive response signature and/or proliferation status. In some cases, a reference set of known gene expression values are obtained from individuals who have been diagnosed with a particular anti-folate predictive response signature and/or proliferation status and are also known to respond (or not respond) to anti-folate therapy. In some cases, a reference set of known gene expression values are obtained from individuals who have been diagnosed with a particular anti-folate predictive response signature and/or proliferation status and are also known to respond (or not respond) to angiogenesis inhibitor therapy. In some cases, a reference set of known gene expression values are obtained from individuals who have been diagnosed with a particular anti-folate predictive response signature and/or proliferation status and are also known to respond (or not respond) to immunotherapy. In some cases, a reference set of known gene expression values are obtained from individuals who have been diagnosed with a particular anti-folate predictive response signature and/or proliferation status and are also known to respond (or not respond) to chemotherapy.

Algorithms suitable for categorization of samples include but are not limited to k-nearest neighbor algorithms, support vector machines, linear discriminant analysis, diagonal linear discriminant analysis, updown, naive Bayesian algorithms, neural network algorithms, hidden Markov model algorithms, genetic algorithms, or any combination thereof.

When a binary classifier is compared with actual true values (e.g., values from a biological sample), there are typically four possible outcomes. If the outcome from a prediction is p (where “p” is a positive classifier output, such as the presence of a deletion or duplication syndrome) and the actual value is also p, then it is called a true positive (TP); however, if the actual value is n then it is said to be a false positive (FP). Conversely, a true negative has occurred when both the prediction outcome and the actual value are n (where “n” is a negative classifier output, such as no deletion or duplication syndrome), and false negative is when the prediction outcome is n while the actual value is p. In one embodiment, consider a test that seeks to determine whether a person is likely or unlikely to respond to angiogenesis inhibitor therapy. A false positive in this case occurs when the person tests positive, but actually does respond. A false negative, on the other hand, occurs when the person tests negative, suggesting they are unlikely to respond, when they actually are likely to respond. The same holds true for classifying a anti-folate predictive response signature or proliferation status.

The positive predictive value (PPV), or precision rate, or post-test probability of disease, is the proportion of subjects with positive test results who are correctly diagnosed as likely or unlikely to respond or diagnosed with the correct anti-folate predictive response signature or proliferation status, or a combination thereof. It reflects the probability that a positive test reflects the underlying condition being tested for. Its value does however depend on the prevalence of the disease, which may vary. In one example the following characteristics are provided: FP (false positive); TN (true negative); TP (true positive); FN (false negative). False positive rate (α)=FP/(FP+TN)−specificity; False negative rate (β)=FN/(TP+FN)−sensitivity; Power=sensitivity=1−β; Likelihood-ratio positive=sensitivity/(1−specificity); Likelihood-ratio negative=(1−sensitivity)/specificity. The negative predictive value (NPV) is the proportion of subjects with negative test results who are correctly diagnosed.

In some embodiments, the results of the biomarker level analysis of the subject methods provide a statistical confidence level that a given diagnosis is correct. In some embodiments, such statistical confidence level is at least about, or more than about 85%, 90%, 91%, 92%, 93%, 94%, 95%, 96%, 97%, 98%, 99% 99.5%, or more.

In some embodiments, the method further includes classifying the sample as a particular LUAD subtype based on the comparison of biomarker levels in the sample and reference biomarker levels, for example present in at least one training set. In some embodiments, the sample is classified as a particular subtype (i.e., bronchioid or non-bronchioid) if the results of the comparison meet one or more criterion such as, for example, a minimum percent agreement, a value of a statistic calculated based on the percentage agreement such as (for example) a kappa statistic, a minimum correlation (e.g., Pearson's correlation) and/or the like.

In some embodiments, the method further includes classifying the sample as a particular level of proliferation based on the comparison of biomarker levels in the sample and reference biomarker levels, for example present in at least one training set. In some embodiments, the sample is classified as a particular level of proliferation if the results of the comparison meet one or more criterion such as, for example, a minimum percent agreement, a value of a statistic calculated based on the percentage agreement such as (for example) a kappa statistic, a minimum correlation (e.g., Pearson's correlation) and/or the like.

It is intended that the methods described herein can be performed by software (stored in memory and/or executed on hardware), hardware, or a combination thereof. Hardware modules may include, for example, a general-purpose processor, a field programmable gate array (FPGA), and/or an application specific integrated circuit (ASIC). Software modules (executed on hardware) can be expressed in a variety of software languages (e.g., computer code), including Unix utilities, C, C++, Java™, Ruby, SQL, SAS®, the R programming language/software environment, Visual Basic™, and other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

Some embodiments described herein relate to devices with a non-transitory computer-readable medium (also can be referred to as a non-transitory processor-readable medium or memory) having instructions or computer code thereon for performing various computer-implemented operations and/or methods disclosed herein. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to: magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which can include, for example, the instructions and/or computer code discussed herein.

In some embodiments, a single biomarker, or from about 5 to about 10, from about 8 to about 16, from about 5 to about 15, from about 5 to about 20, from about 5 to about 25, from about 5 to about 30, from about 5 to about 35, from about 5 to about 40, from about 5 to about 45, from about 5 to about 48 biomarkers (e.g., as disclosed in Table 1) is capable of classifying an anti-folate predictive response signature with a predictive success of at least about 70%, at least about 71%, at least about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, up to 100%, and all values in between. In some embodiments, any combination of biomarkers disclosed herein (e.g., in Table 1) can be used to obtain a predictive success of at least about 70%, at least about 71%, at least about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, up to 100%, and all values in between.

In some embodiments, a single biomarker, or from about 5 to about 10, from about 8 to about 16, from about 5 to about 15, from about 5 to about 20, from about 5 to about 25, from about 5 to about 30, from about 5 to about 35, from about 5 to about 40, from about 5 to about 45, from about 5 to about 48 biomarkers (e.g., as disclosed in Table 1) is capable of classifying an anti-folate predictive response signature with a sensitivity or specificity of at least about 70%, at least about 71%, at least about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, up to 100%, and all values in between. In some embodiments, any combination of biomarkers disclosed herein can be used to obtain a sensitivity or specificity of at least about 70%, at least about 71%, at least about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, up to 100%, and all values in between.

In some embodiments, a single biomarker, or from about 2 to about 4, from about 4 to about 6, from about 6 to about 8, from about 8 to about 10, from about 10 to about 12, from about 12 to about 14, from about 14 to about 16, from about 16 to about 18, from about 20 to about 22, from about 22 to about 24 biomarkers or from about 24 to about 26 biomarkers (e.g., as disclosed in Table 2) is capable of classifying the presence, absence, level of proliferation with a predictive success of at least about 70%, at least about 71%, at least about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, up to 100%, and all values in between. In some embodiments, any combination of biomarkers disclosed herein (e.g., in Table 2) can be used to obtain a predictive success of at least about 70%, at least about 71%, at least about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, up to 100%, and all values in between.

In some embodiments, a single biomarker, or from about 2 to about 4, from about 4 to about 6, from about 6 to about 8, from about 8 to about 10, from about 10 to about 12, from about 12 to about 14, from about 14 to about 16, from about 16 to about 18, from about 20 to about 22, from about 22 to about 24 biomarkers or from about 24 to about 26 biomarkers (e.g., as disclosed in Table 2) is capable of classifying the presence, absence, level of proliferation with a sensitivity or specificity of at least about 70%, at least about 71%, at least about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, up to 100%, and all values in between. In some embodiments, any combination of biomarkers disclosed herein can be used to obtain a sensitivity or specificity of at least about 70%, at least about 71%, at least about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, up to 100%, and all values in between.

### Prognostic Uses

In one aspect, provided herein is a method for determining a disease outcome in a subject suffering from or suspected of suffering from cancer. The cancer can be any cancer known in the art and/or provided herein. In one embodiment, the subject is suffering from or suspected of suffering from a cancer selected from KIRP, BRCA, THCA, BLCA, PRAD, KICH, CESC, KIRC, LIHC, LGG, SARC, LUAD, COAD, HNSC, UCEC, GBM. ESCA, STAD, OV or READ. The disease outcome can be a prognosis. The prognostic information that can be obtained by the methods provided herein can comprise a number of possible endpoints, which can be selected from time from surgery to distant metastases (distant recurrence-free survival), time of disease-free survival (recurrence free survival), and time of overall survival. In some cases, Kaplan-Meier plots (Kaplan and Meier. J Am Stat Assoc 53: 457-481 (1958)) can be used to display time-to-event curves for any or all of these three endpoints. In some cases, a cox regression (or proportional hazards regression) can be performed in order to determine a hazard ratio for any or all of these three endpoints. In one embodiment, a cox regression (or proportional hazards regression) is used to assess the prognostic performance in terms of overall survival of the proliferation score or signature of sample as determined using the methods provided herein. The Cox Proportional Hazards analysis is a regression method for survival data that provides an estimate of the hazard ratio and its confidence interval. The Cox model is a well-recognized statistical technique for exploring the relationship between the survival of a subject and particular variables. This statistical method permits estimation of the hazard (i.e., risk) of individuals given their prognostic variables (e.g., proliferation status with or without other additional clinical factors, as described herein). The “hazard ratio” is the risk of death at any given time point for patients displaying particular prognostic variables. See generally Spruance et al., Antimicrob. Agents & Chemo. 48:2787-92 (2004). The additional clinical factors can include age, sex, tumor diameter, tumor stage and smoking history. A relevant time interval or time point can be at least 1 year, at least two years, at least three years, at least five years, or at least ten years.

In one embodiment, the method for determining a disease outcome for a subject suffering from or suspected of suffering from a cancer can comprise: (a) determining an anti-folate predictive response signature of a sample obtained from the subject, wherein the determining the anti-folate predictive response signature comprises measuring an expression level of at least five classifier genes from a plurality of classifier genes in Table 1 in the sample obtained from the subject; (b) determining an anti-folate predictive response signature of a control sample, wherein the determining the anti-folate predictive response signature comprises measuring an expression level of at least five classifier genes from a plurality of classifier genes in Table 1 in the control sample, wherein the at least five classifier genes are identical to those measured for the sample obtained from the subject; and (c) comparing the anti-folate predictive response signature of the sample obtained from the subject to the anti-folate predictive response signature of the control sample. In one embodiment, the control sample can be from a bronchioid cancer sample. In another embodiment, the control sample can be a non-bronchioid cancer sample. Further to either of these embodiments, a positive anti-folate predictive response signature in the sample obtained from the subject as compared to the control sample can be indicative of a poor disease outcome for the subject. In still another embodiment, a negative anti-folate predictive response signature in the sample obtained from the subject as compared to the control sample can be indicative of a poor disease outcome for the subject. The expression level of any and all classifier genes can be normalized as provided herein, such as, for example, normalizing expression of the classifier genes by using expression levels from one or more reference or housekeeping genes.

In one embodiment, the method for determining a disease outcome for a subject suffering from or suspected of suffering from a cancer can comprise: (a) determining a proliferation signature of a sample obtained from the subject, wherein the determining the proliferation signature comprises measuring an expression level of at least five classifier genes from a plurality of classifier genes in Table 2 in the sample obtained from the subject; (b) determining a proliferation signature of a control sample, wherein the determining the proliferation signature comprises measuring an expression level of at least five classifier genes from a plurality of classifier genes in Table 2 in the control sample, wherein the at least five classifier genes are identical to those measured for the sample obtained from the subject; and (c) comparing the proliferation signature of the sample obtained from the subject to the proliferation signature of the control sample. In one embodiment, the control sample can be from a healthy subject. In another embodiment, the control sample can be a non-proliferative cancer sample. Further to either of these embodiments, an elevated proliferation score in the sample obtained from the subject as compared to the control sample can be indicative of a poor disease outcome for the subject. In still another embodiment, the control sample can be a proliferative cancer sample. Further to this embodiment, a similar or elevated proliferation score in the sample obtained from the subject as compared to the control sample can be indicative of a poor disease outcome for the subject, while a reduced proliferation score can be indicative of a good or better prognosis. The expression level of any and all classifier genes can be normalized as provided herein, such as, for example, normalizing expression of the classifier genes by using expression levels from one or more reference or housekeeping genes.

In another embodiment, the method for determining a disease outcome for a subject suffering from or suspected of suffering from a cancer can comprise: (a) determining a proliferation score of a sample obtained from the subject, wherein the determining the proliferation score comprises: (i) measuring an expression level of at least five classifier genes from a plurality of classifier genes in Table 2 in the sample obtained from the subject; and (ii) calculating a mean expression level for the at least five classifier biomarkers from the plurality of classifier biomarkers, wherein the mean expression level for the at least five classifier biomarkers from the plurality of classifier biomarkers represents the proliferation score; (b) determining a proliferation score of a control sample, wherein the determining the proliferation score comprises: (i) measuring a expression level of at least five classifier genes from a plurality of classifier genes in Table 2 in the control sample; and (ii) calculating a mean expression level for the at least five classifier biomarkers from the plurality of classifier biomarkers, wherein the mean expression level for the at least five classifier biomarkers from the plurality of classifier biomarkers represents the proliferation score; and (c) comparing the proliferation score of the sample obtained from the subject to the proliferation score of the control sample. In one embodiment, the control sample can be from a healthy subject. In another embodiment, the control sample can be a non-proliferative cancer sample. Further to either of these embodiments, an elevated proliferation score in the sample obtained from the subject as compared to the control sample can be indicative of a poor disease outcome for the subject. In still another embodiment, the control sample can be a proliferative cancer sample. Further to this embodiment, a similar or elevated proliferation score in the sample obtained from the subject as compared to the control sample can be indicative of a poor disease outcome for the subject, while a reduced proliferation score can be indicative of a good or better prognosis. The expression level of any and all classifier genes can be normalized as provided herein, such as, for example, normalizing expression of the classifier genes by using expression levels from one or more reference or housekeeping genes.

Further to any of the above embodiments related to assessing proliferation, the plurality of classifier genes can be a gene or set of genes known in the art known to play a role in proliferation and/or mitosis. For example, the set of classifier genes can be the set of 11-genes found in Nielsen T O et al., A comparison of PAM50 intrinsic subtyping with immunohistochemistry and clinical prognostic factors in tamoxifen-treated estrogen receptor positive breast cancer. Clin Cancer Res 16(21):5222-5232, or the set of 18 genes found in Walden et al, 2015 PMID: 26297356, and United States Patent Application 20130337444, each of which are incorporated herein by reference. In one embodiment, the plurality of classifier genes is one or a plurality of genes from Table 2. In another embodiment, the plurality of classifier genes is one or a plurality of genes from Table 2 in combination with any other set of classifier genes known in the art and/or provided herein to play a role in proliferation and/or mitosis. Further any of the above embodiments, the method for determining a disease outcome can comprise or consist of measuring the expression level is of at least 10, 15, 20 or 25 classifier genes from the plurality of classifier genes found in Table 2. In some cases, the method comprises or consist of measuring the expression level of all of the classifier genes from the plurality of classifier genes found in Table 2.

Further any of the above embodiments, the sample obtained from the subject and/or the control sample is a formalin-fixed, paraffin-embedded (FFPE) tissue sample, fresh or a frozen tissue sample, an exosome, wash fluids, cell pellets, or a bodily fluid obtained from the patient. In one embodiment, the sample obtained from the subject and/or the control is an FFPE tissue sample. In another embodiment, the sample obtained from the subject and/or the control is a fresh frozen tissue sample.

Further any of the above embodiments, the expression level can be a nucleic acid or protein expression level. The nucleic acid or protein expression level can be measured using any method known in the art and/or provided herein. In one embodiment, the method of determining the presence of metastatic disease as provided herein entails measuring a nucleic acid expression level. The nucleic acid expression level can be measured using an amplification, sequencing or hybridization assay. The amplification, hybridization and/or sequencing assay can comprise performing quantitative real time reverse transcriptase polymerase chain reaction (qRT-PCR), RNA-seq, microarrays, gene chips, nCounter Gene Expression Assay, Serial Analysis of Gene Expression (SAGE), Rapid Analysis of Gene Expression (RAGE), nuclease protection assays, Northern blotting, nCounter DX Analysis System or any other equivalent gene expression detection techniques. In one embodiment, the nucleic acid expression level is detected by performing RNA-seq.

Further to any of the embodiments related to determining disease outcome in a subject, the method can further comprise determining a level and/or activity of at least one additional marker involved in cell proliferation and mitosis. The one additional marker can be any marker known in the art and/or provided herein as playing a role in proliferation or mitosis. The additional marker can be selected from the group consisting of Ki-67, CD31, KIFC1 (kinesin family member C1), KIF2C (kinesin family member 2C), KIF14 (kinesin family member 14), CCNB2 (cyclin B2), SIL (SCL-TAL1 interrupting locus) and TNPO1 (transportin I). In one embodiment, the additional marker is Ki67 or CD31.

In one embodiment, the method for determining disease outcome as provided herein further comprises determining a subtype of the sample obtained from the subject. The subtype can be determined via histological examination of the sample. The subtype can be determined via gene expression analysis of the sample. The gene expression analysis of the sample is performed using a gene expression sub-typer that is publically available and/or provided herein. The gene expression-based cancer subtyping can be determined using gene signatures known in the art for specific types of cancer. In one embodiment, the cancer is lung cancer, and the gene signature is selected from the gene signatures found in WO2017/201165, WO2017/201164, US20170114416 or U.S. Pat. No. 8,822,153, each of which is herein incorporated by reference in their entirety. In one embodiment, the cancer is head and neck squamous cell carcinoma (HNSCC) and the gene signature is selected from the gene signatures found in PCT/US18/45522 or PCT/US18/48862, each of which is herein incorporated by reference in their entirety. In one embodiment, the cancer is breast cancer, and the gene signature is the PAM50 subtyper found in Parker J S et al., (2009) Supervised risk predictor of breast cancer based on intrinsic subtypes. J Clin Oncol 27:1160-1167, which is herein incorporated by reference in its entirety.

### Risk of Recurrence

Also provided herein is a method for determining a risk of recurrence of a cancer in a subject suffering therefrom. This risk of recurrence as determined by the methods provided herein may be further combined with other prognostic factors such as age, sex, tumor diameter and smoking history in order to provide additional prognostic information. The cancer can be any cancer known in the art and/or provided herein. In one embodiment, the subject is suffering from or suspected of suffering from a cancer selected from KIRP, BRCA, THCA, BLCA, PRAD, KICH, CESC, KIRC, LIHC, LGG, SARC, LUAD, COAD, HNSC, UCEC, GBM, ESCA, STAD, OV or READ.

The method for determining the risk of recurrence in said subject can comprise or consist of measuring an expression level of at least five classifier genes from a plurality of classifier genes in a first sample obtained from the subject, measuring the expression level of the same at least five classifier genes from the plurality of classifier genes in a control sample, wherein the expression level of the at least five classifier genes represents a proliferation signature of the control sample, and determining existence of a correlation between the proliferation signature of the first sample and the proliferation signature of the control sample. The expression level can be normalized as provided herein, such as, for example, normalizing expression of the classifier genes by using expression levels from one or more reference or housekeeping genes. In one embodiment, prior to determining the existence of a correlation between the proliferation signature of the first sample and the proliferation signature of the control sample, the method comprises or consist of determining a proliferation score for the first sample and the control sample. Determining the proliferation score can comprise determining a mean nucleic acid expression level for the at least five classifier biomarkers from the plurality of classifier biomarkers for the first sample and the control sample. Determining the existence of a correlation can entail determining the existence of a correlation between the proliferation score of the first sample and the proliferation score of the control sample.

The correlation between the first sample and the control sample can be performed in a various ways. The correlation can be determined using any statistical test or algorithm known in the art that is appropriate for such an analysis. In some cases, a correlation coefficient is determined that is a measure of the similarity of dissimilarity of the first sample with said control sample. A number of different coefficients can be used for determining a correlation between the expression level in the first sample from the subject and the control sample. In some cases, the methods for determining a correlation coefficient are parametric methods, which assume a normal distribution of the data. One of these methods can be the Pearson product-moment correlation coefficient, which can be obtained by dividing the covariance of the two variables by the product of their standard deviations. Other methods can comprise cosine-angle, un-centered correlation and, more preferred, cosine correlation (Fan et al., Conf Proc IEEE Eng Med Biol Soc. 5:4810-3 (2005)). In some cases, the methods for determining a correlation coefficient are non-parametric methods such as, for example, methods for determining a Kendall correlation or a Spearman correlation.

Correlation can be a bivariate analysis that measures the strength of association between two variables and the direction of the relationship. In some cases, said correlation of the first sample with the second sample can be used to produce an overall similarity score for the set of classifier genes (e.g., from Table 2) that are used. A similarity score can be a measure of the average correlation of the expression levels of the one or plurality of classifier genes (e.g., from Table 2) in the first sample from the subject and the control sample. Said similarity score can be a numerical value between +1, indicative of a high correlation between the expression levels of the one or plurality of classifier genes (e.g., from Table 2) in the first sample from the subject and the control sample, and −1 (van't Veer et al., Nature 415: 484-5 (2002)).

In one embodiment, the control sample is obtained from the subject such that the first and control samples are obtained from different regions of the subject's body such that the control sample is from an area of the subject's body that is normal (i.e., not cancerous). In another embodiment, the control sample is obtained from a control subject that does not have the type of cancer the subject is suffering or suspected of suffering from. Further to this embodiment, the control sample obtained from the control subject can be from the same area of the body as the first sample. In yet another embodiment, the control sample is obtained from a control subject that does have the same type of cancer that the subject is suffering from or suspected of suffering from but said control sample has been deemed to have a low risk of recurrence. In still another embodiment, the control sample is obtained from a control subject that does have the same type of cancer that the subject is suffering from or suspected of suffering from and said control sample has been deemed to have a high or increased risk of recurrence. Further to this embodiment, the control sample obtained from the control subject can be from the same area of the body as the first sample. The first and/or control sample can be a formalin-fixed, paraffin-embedded (FFPE) tissue sample, fresh or a frozen tissue sample, an exosome, wash fluids, cell pellets, or a bodily fluid obtained from the subject. In one embodiment, the first sample and the control sample is an FFPE tissue sample. In another embodiment, the first sample and the control sample is a fresh frozen tissue sample.

In one embodiment, a similarity score is determined as provided herein and an arbitrary threshold is determined for said similarity score. In embodiments wherein the control sample is from another or different part of the subject's body that is not cancerous or is from a subject that does not have the same type of cancer as the subject, first samples that score below said threshold are indicative of an increased risk of recurrence, while first samples that score above said threshold are indicative of a low risk of recurrence. In embodiments wherein the control sample is from a subject that does have the same type of cancer and has a low risk of recurrence, first samples that score below said threshold are indicative of an increased risk of recurrence, while first samples that score above said threshold are indicative of a low risk of recurrence. In embodiments wherein the control sample is from a subject that does have the same type of cancer and has an increased risk of recurrence, first samples that score below said threshold are indicative of a decreased risk of recurrence, while first samples that score above said threshold are indicative of a high risk of recurrence.

In some cases, the method for determining the risk of recurrence comprises or consists of measuring the expression level is of at least 10, 15, 20 or 25 classifier genes from the plurality of classifier genes. In some cases, the method comprises or consists of measuring the expression level of all of the classifier genes from the plurality of classifier genes. In one embodiment, the plurality of classifier genes are the classifier genes found in Table 2. In another embodiment, the method comprises or consists of measuring the expression level of at least 10%, 15%, 20%, 25%, 30%, 35%, 40%, 45%, 50%, 55%, 60%, 65%, 70%, 75%, 80%, 85%, 90%, 95% or 99% of the classifiers from Table 2. In another embodiment, the method comprises or consists of measuring the expression level of about 10%, 15%, 20%, 25%, 30%, 35%, 40%, 45%, 50%, 55%, 60%, 65%, 70%, 75%, 80%, 85%, 90%, 95% or 99% of the classifiers from Table 2. In another embodiment, the method comprises or consists of measuring the expression level of at most 10%, 15%, 20%, 25%, 30%, 35%, 40%, 45%, 50%, 55%, 60%, 65%, 70%, 75%, 80%, 85%, 90%, 95% or 99% of the classifiers from Table 2.

The expression level can be a nucleic acid or protein expression level. The nucleic acid or protein expression level can be measured using any method known in the art and/or provided herein. In one embodiment, the method of determining the presence of metastatic disease as provided herein entails measuring a nucleic acid expression level. The nucleic acid expression level can be measured using an amplification, sequencing or hybridization assay. The amplification, hybridization and/or sequencing assay can comprise performing quantitative real time reverse transcriptase polymerase chain reaction (qRT-PCR), RNA-seq, microarrays, gene chips, nCounter Gene Expression Assay, Serial Analysis of Gene Expression (SAGE), Rapid Analysis of Gene Expression (RAGE), nuclease protection assays, Northern blotting, nCounter DX Analysis System or any other equivalent gene expression detection techniques. In one embodiment, the nucleic acid expression level is detected by performing RNA-seq.

In one embodiment, the method for determining risk of recurrence as provided herein further comprises determining a subtype of the sample obtained from the subject. The subtype can be determined via histological examination of the sample. The subtype can be determined via gene expression analysis of the sample. The gene expression analysis of the sample is performed using a gene expression sub-typer that is publically available and/or provided herein. The gene expression-based cancer subtyping can be determined using gene signatures known in the art for specific types of cancer. In one embodiment, the cancer is lung cancer, and the gene signature is selected from the gene signatures found in WO2017/201165, WO2017/201164, US20170114416 or U.S. Pat. No. 8,822,153, each of which is herein incorporated by reference in their entirety. In one embodiment, the cancer is head and neck squamous cell carcinoma (HNSCC) and the gene signature is selected from the gene signatures found in PCT/US18/45522 or PCT/US18/48862, each of which is herein incorporated by reference in their entirety. In one embodiment, the cancer is breast cancer, and the gene signature is the PAM50 subtyper found in Parker J S et al., (2009) Supervised risk predictor of breast cancer based on intrinsic subtypes. J Clin Oncol 27:1160-1167, which is herein incorporated by reference in its entirety.

In one embodiment, the intrinsic subtype of a sample can be combined with the proliferation score of the sample in order to calculate a risk of recurrence (ROR) score for a subject. The ROR score can be calculated as described in US20130337444, which is herein incorporated by reference.

### Clinical/Therapeutic Uses

In one embodiment, the method as provided herein for determining the anti-folate predictive response signature of a sample (e.g., using RNA sequencing data obtained from the sample) obtained from a subject suffering or suspected of suffering from cancer is used to determine whether or not said subject is a candidate for treatment with a specific type or types of cancer therapy (e.g., anti-folate therapy). In one embodiment, the method as provided herein for assessing proliferation in a sample (e.g., using RNA sequencing data obtained from the sample) from a subject suffering or suspected of suffering from cancer is used to determine whether or not said subject is a candidate for treatment with a specific type or types of cancer therapy. The sample can be any type of sample obtained from the subject as provided herein. The cancer can be any type of cancer known in the art and/or provided herein. In one embodiment, determining the anti-folate predictive response signature is one of a number of methods that can be employed to characterize the sample obtained from the patient such that the determining the anti-folate predictive response signature alone or in combination with one or more of the number of methods can be used to determine whether or not said patient is a candidate for treatment with a specific type or types of cancer therapy (e.g., anti-folate therapy). In one embodiment, assessing proliferation is one of a number of methods that can be employed to characterize the sample obtained from the patient such that the assessing proliferation alone or in combination with one or more of the number of methods can be used to determine whether or not said patient is a candidate for treatment with a specific type or types of cancer therapy. In addition to assessing or determining an anti-folate predictive response signature, the number of methods for characterizing the sample can entail determining the presence, absence or level of proliferation, the proliferation score, the tumor mutation burden (TMB), the subtype, the level of immune activation or any combination thereof. The characterization can be performed on RNA sequencing data obtained from the sample. In addition to assessing or determining a proliferation score, the number of methods for characterizing the sample can entail determining the anti-folate predictive response signature, the tumor mutation burden (TMB), the subtype, the level of immune activation or any combination thereof. The characterization can be performed on RNA sequencing data obtained from the sample.

In one embodiment, in addition to determining the anti-folate predictive response signature and/or assessing proliferation as provided herein, the characterization entails calculating a TMB value and/or rate. In one embodiment, the TMB value and/or rate can be calculated from RNA (e.g., via transcriptome profiling or RNA sequencing)) as provided in PCT/US2019/055322 Oct. 9, 2019, which is herein incorporated by reference herein.

The determination of whether or not said patient is a candidate for treatment with a specific type or types of cancer therapy can be based on the anti-folate predictive response signature alone, the proliferation signature and/or calculated proliferation score alone or in combination with other methods known in the art for characterizing a sample obtained from a subject suffering from or suspected of suffering from cancer. The other methods for characterizing said sample can be histologically based methods, gene expression-based methods or a combination thereof. The histologically based methods can include histological cancer subtyping by one or more trained pathologists as well as the histological based methods of assessing proliferation such as, for example, determining the mitotic activity index. The gene expression-based methods can include subtyping, assessment of MSI, assessment of TMB, assessment of cell of origin, immune subtyping, assessing tumor purity or any combination thereof. The gene expression-based methods can be assessed from DNA, RNA or a combination thereof. In one embodiment, the characterization of the sample obtained from the patient suffering from or suspected of suffering from cancer is performed on RNA obtained or isolated from the sample.

The gene expression-based cancer subtyping can be determined using gene signatures known in the art for specific types of cancer. In one embodiment, the cancer is lung cancer, and the gene signature is selected from the gene signatures found in WO2017/201165, WO2017/201164, US20170114416 or U.S. Pat. No. 8,822,153, each of which is herein incorporated by reference in their entirety. In one embodiment, the cancer is head and neck squamous cell carcinoma (HNSCC) and the gene signature is selected from the gene signatures found in PCT/US18/45522 or PCT/US18/48862, each of which is herein incorporated by reference in their entirety. In one embodiment, the cancer is breast cancer, and the gene signature is the PAM50 subtyper found in Parker J S et al., (2009) Supervised risk predictor of breast cancer based on intrinsic subtypes. J Clin Oncol 27:1160-1167, which is herein incorporated by reference in its entirety.

The gene expression based immune subtyping or immune cell activation can be determined using immune expression signatures known in the art such as, for example, the gene signatures found in Thorsson, V., Gibbs, D. L., Brown, S. D., Wolf, D., Bortone, D. S., Yang, T. H. O., Porta-Pardo, E., Gao, G. F., Plaisier, C. L., Eddy, J. A. and Ziv, E., 2018, The immune landscape of cancer. Immunity, 48(4), pp.812-830, which is herein incorporated by reference in its entirety. In some embodiments, immune cell signatures can also include (Table 2) (Bindea G. et al., Immunity, 39(4): 782-95 (2013); Faruki H. et al., JTO, 12(6): 943-953 (2017); Charoentong P. et al., Cell reports, 18, 248-262 (2017,) the contents of each of which are herein incorporated by reference in its entirety. In one embodiment, the method further comprises measuring single gene immune biomarkers, such as, for example, CTLA4, PDCD1 and CD274 (PD-LI), PDCDLG2(PD-L2) and/or IFN gene signatures. In one embodiment, the level of immune cell activation is determined by measuring gene expression signatures of immunomarkers. The immunomarkers can be measured in the same and/or different sample used to determine the proliferation signature or score as described herein. The immunomarkers can be those found in WO2017/201165, and WO2017/201164, each of which is herein incorporated by reference in their entirety.

In one embodiment, characterizing a tumor sample further entails an additional set of biomarker classifiers that can include assessing tumor purity ABSOLUTE derived from the TCGA supplementary data.

In one embodiment, an additional set of biomarker classifiers can include a 5 gene signature comprising tumor driver genes such as TP53 and RB1, and receptor tyrosine kinases including FGFR2, FGFR3, and ERBB2. In one embodiment, the 5 gene signature is related to the signature of tumor driver genes.

In one embodiment, upon determining a subject's anti-folate predictive response signature alone, proliferation signature or score alone or in combination with other characterization methods as described herein (e.g., cancer subtype, MSI, immune subtype and/or TMB status), the subject is selected for a specific therapy, for example, anti-folate therapy, radiotherapy (radiation therapy), surgical intervention, target therapy, chemotherapy or drug therapy with an angiogenesis inhibitor or immunotherapy or combinations thereof. In some embodiments, the specific therapy can be any treatment or therapeutic method that can be used for a cancer patient. In one embodiment, upon determining a subject's anti-folate predictive response signature, proliferation signature or score or anti-folate predictive response signature in combination with proliferation signature or score, the subject is administered a suitable therapeutic agent, for example, an anti-folate agent, chemotherapeutic agent(s) or an angiogenesis inhibitor or immunotherapeutic agent(s). In one embodiment, the therapy is anti-folate therapy, and the anti-folate agent is pemetrexed, methotrexate, trimetrexate, lometrexol, raltitrexed and nolatrexed. In one embodiment, the therapy is immunotherapy, and the immunotherapeutic agent is a checkpoint inhibitor, monoclonal antibody, biological response modifier, therapeutic vaccine or cellular immunotherapy. In some embodiments, the determination of a suitable treatment can identify treatment responders. In some embodiments, the determination of a suitable treatment can identify treatment non-responders. In some embodiments, upon determining a patient's proliferation signature or score, the patient can be selected for any combination of suitable therapies. For example, chemotherapy or drug therapy with a radiotherapy, a surgical intervention with an immunotherapy or a chemotherapeutic agent with a radiotherapy. In some embodiments, immunotherapy, or immunotherapeutic agent can be a checkpoint inhibitor, monoclonal antibody, biological response modifier, therapeutic vaccine or cellular immunotherapy.

The methods of present invention are also useful for evaluating clinical response to therapy, as well as for endpoints in clinical trials for efficacy of new therapies.

In one embodiment, the methods of the invention also find use in predicting response to different lines of therapies based on the anti-folate predictive response signature alone, the proliferation signature or score alone, the anti-folate predictive response signature in combination with proliferation signature or score alone or in combination with other characterization methods as described herein (e.g., cancer subtype, immune subtype and/or TMB status). For example, response to anti-folate therapy can be improved by more accurately assigning the anti-folate predictive response signature and/or proliferation signature or score. For example, chemotherapeutic response can be improved by more accurately assigning proliferation signature or score. Likewise, treatment regimens can be formulated based on the anti-folate predictive response signature alone, the proliferation signature or score alone, anti-folate predictive response signature in combination with proliferation signature or score or in combination with other characterization methods as described herein (e.g., cancer subtype, immune subtype and/or TMB status).

### Anti-Folate Agents

In one embodiment, provided herein is a method of determining whether a patient suffering from cancer is likely to respond to treatment with an antifolate agent. The method can comprising: determining an antifolate predictive response signature of a sample obtained from a patient suffering from cancer and, based on the antifolate predictive response signature, assessing or determining whether the patient is likely to respond to treatment with an antifolate agent. In one embodiment, a positive antifolate predictive response signature predicts that the patient is likely to respond to the treatment with an antifolate agent. In one embodiment, a negative antifolate predictive response signature predicts that the patient is unlikely to respond to the treatment with an antifolate agent. A patient unlikely to respond to treatment with an antifolate agent may be a candidate for treatment with another agent such as, an angiogenesis inhibitor, immunotherapy, radiotherapy, surgical intervention, etc. Determination of whether or not a patient unlikely to respond to treatment with an antifolate agent may be responsive to any other cancer treatment known in the art and/or provided herein can be based on further or additional molecular characterization of the sample. The additional molecular characterization can entail any of the molecular analyses described herein. The cancer can be any cancer known in the art and/or provided herein. The sample can be any type of sample as provided herein such as, for example, a tumor sample. The anti-folate agent is selected from pemetrexed, methotrexate, trimetrexate, lometrexol, raltitrexed and nolatrexed. In one embodiment, the antifolate agent is pemetrexed. In one embodiment, the antifolate agent is raltitrexed. The determining the antifolate predictive response signature of the sample obtained from the patient suffering from cancer can comprise determining expression levels of a plurality of classifier biomarkers. In one embodiment, the plurality of classifier biomarkers for determining the antifolate predictive response signature is selected from Table 1. In one embodiment, the method further comprises comparing the detected levels of expression of the plurality of classifier biomarkers of Table 1 to the expression of the plurality of classifier biomarkers of Table 1 in at least one sample training set(s), wherein the at least one sample training set comprises expression data of the plurality of classifier biomarkers of Table 1 from a reference adenocarcinoma TRU (bronchioid) sample, expression data of the plurality of classifier biomarkers of Table 1 from a reference adenocarcinoma PP (magnoid) sample, expression data of the plurality of classifier biomarkers of Table 1 from a reference adenocarcinoma PI (squamoid) sample, or a combination thereof; and classifying the sample as TRU, or non-TRU (i.e., PP, or PI) based on the results of the comparing step. The comparing step can comprise applying a statistical algorithm which comprises determining a correlation between the expression data obtained from the sample and the expression data from the at least one training set(s); and classifying the sample as a TRU, or non-TRU (i.e., PP and/or PI) subtype based on the results of the statistical algorithm. In one embodiment, the method further comprises determining the expression level of one or more anti-folate drug targets in the tumor sample obtained from the patient. The one or more anti-folate drug targets can be selected from DHFR, GART, TYMS, ATIC, or MTHFD1L genes. The method can further comprise determining a tumor mutational burden of the tumor sample obtained from the patient. The method can further comprise determining a proliferation signature of the tumor sample obtained from the patient. The proliferation signature can be determined using any of the methods provided herein that utilize the biomarkers of Table 2. In one embodiment, a sample obtained from subject suffering from cancer that possesses a low proliferation score or a proliferation signature indicative of a low amount of proliferation as compared to a control can be indicative of subject who is likely to respond to treatment with an anti-folate agent.

In another embodiment, also provided herein is method for selecting a patient suffering from cancer for an antifolate agent. The method can comprise determining an antifolate predictive response signature of a sample obtained from a patient suffering from cancer and selecting the patient for treatment with an antifolate agent if the antifolate response signature is positive. The cancer can be any cancer known in the art and/or provided herein. The sample can be any type of sample as provided herein such as, for example, a tumor sample. The anti-folate agent is selected from pemetrexed, methotrexate, trimetrexate, lometrexol, raltitrexed and nolatrexed. In one embodiment, the antifolate agent is pemetrexed. In one embodiment, the antifolate agent is raltitrexed. The determining the antifolate predictive response signature of the sample obtained from the patient suffering from cancer can comprise determining expression levels of a plurality of classifier biomarkers. In one embodiment, the plurality of classifier biomarkers for determining the antifolate predictive response signature is selected from Table 1. In one embodiment, the method further comprises comparing the detected levels of expression of the plurality of classifier biomarkers of Table 1 to the expression of the plurality of classifier biomarkers of Table 1 in at least one sample training set(s), wherein the at least one sample training set comprises expression data of the plurality of classifier biomarkers of Table 1 from a reference adenocarcinoma TRU (bronchioid) sample, expression data of the plurality of classifier biomarkers of Table 1 from a reference adenocarcinoma PP (magnoid) sample, expression data of the plurality of classifier biomarkers of Table 1 from a reference adenocarcinoma PI (squamoid) sample, or a combination thereof; and classifying the sample as TRU, or non-TRU (i.e., PP, or PI) based on the results of the comparing step. The comparing step can comprise applying a statistical algorithm which comprises determining a correlation between the expression data obtained from the sample and the expression data from the at least one training set(s); and classifying the sample as a TRU, or non-TRU (i.e., PP and/or PI) subtype based on the results of the statistical algorithm. In one embodiment, the method further comprises determining the expression level of one or more anti-folate drug targets in the tumor sample obtained from the patient. The one or more anti-folate drug targets can be selected from DHFR, GART, TYMS, ATIC, or MTHFD1L genes. The method can further comprise determining a tumor mutational burden of the tumor sample obtained from the patient. The method can further comprise determining a proliferation signature of the tumor sample obtained from the patient. The proliferation signature can be determined using any of the methods provided herein that utilize the biomarkers of Table 2. In one embodiment, a sample obtained from subject suffering from cancer that possesses a low proliferation score or a proliferation signature indicative of a low amount of proliferation as compared to a control can be indicative of subject who is likely to respond to treatment with an anti-folate agent.

In any of the above embodiments, the determining the expression levels of the plurality of classifier biomarkers can be at a nucleic acid level by performing RNA sequencing, reverse transcriptase polymerase chain reaction (RT-PCR) or hybridization-based analyses. The RT-PCR can be quantitative real time reverse transcriptase polymerase chain reaction (qRT-PCR). The RT-PCR can be performed with primers specific to the classifier biomarkers selected from the plurality of classifier biomarkers of Table 1.

In any of the above embodiments, the TRU (bronchioid) subtype is indicative of a positive antifolate predictive response signature (i.e., AF-PRS (+)), wherein the positive antifolate predictive response signature selects the patient for treatment with an antifolate agent.

The plurality of classifier biomarkers can comprise, consist essentially of or consist of at least 8 biomarker nucleic acids, at least 16 biomarker nucleic acids, at least 32 biomarker nucleic acids, or all 48 biomarker nucleic acids of Table 1.

### Angiogenesis Inhibitors

In one embodiment, upon determining a subject's anti-folate predictive response signature alone, proliferation signature or score alone, anti-folate predictive response signature in combination with proliferation signature or score alone or in combination with other characterization methods as described herein (e.g., cancer subtype, immune subtype and/or TMB status), the patient is selected for drug therapy with an angiogenesis inhibitor.

In one embodiment, the angiogenesis inhibitor is a vascular endothelial growth factor (VEGF) inhibitor, a VEGF receptor inhibitor, a platelet derived growth factor (PDGF) inhibitor or a PDGF receptor inhibitor.

In general, methods of determining whether a patient is likely to respond to angiogenesis inhibitor therapy, or methods of selecting a patient for angiogenesis inhibitor therapy are provided herein. In one embodiment, the method comprises determining an anti-folate predictive response signature alone, a proliferation signature or score alone, an anti-folate predictive response signature in combination with a proliferation signature or score alone, or in combination with other characterization methods as described herein (e.g., cancer subtype, immune subtype and/or TMB status) and probing a sample from the patient for the levels of at least five hypoxia biomarkers selected from the group consisting of RRAGD, FABP5, UCHL1, GAL, PLOD, DDIT4, VEGF, ADM, ANGPTL4, NDRG1, NP, SLC16A3, and C140RF58 (see Table 3) at the nucleic acid level. In a further embodiment, the probing step comprises mixing the sample with five or more oligonucleotides that are substantially complementary to portions of nucleic acid molecules of the at least five biomarkers of Table 1 or Table 2 under conditions suitable for hybridization of the five or more oligonucleotides to their complements or substantial complements, detecting whether hybridization occurs between the five or more oligonucleotides to their complements or substantial complements; and obtaining hybridization values of the sample based on the detecting steps. In embodiments related to determining an anti-folate predictive response signature, the hybridization values of the sample are then compared to reference hybridization value(s) from at least one sample training set, wherein the at least one sample training set comprises (i) hybridization value(s) of the at least five biomarkers from a sample that overexpresses the at least five biomarkers, or overexpresses a subset of the at least five biomarkers, (ii) hybridization values of the at least five biomarkers from a reference bronchioid sample, or (iii) hybridization values of the at least five biomarkers from a non-bronchioid sample. A determination of whether the patient is likely to respond to angiogenesis inhibitor therapy, or a selection of the patient for angiogenesis inhibitor is then made based upon (i) the subject's AF-PRS alone or in combination with other characterization methods as described herein (e.g., proliferation signature or score, cancer subtype, immune subtype and/or TMB status) and (ii) the results of comparison. In embodiments related to assessing proliferation, In embodiments related to determining a proliferation signature or score, the hybridization values of the sample are then compared to reference hybridization value(s) from at least one sample training set, wherein the at least one sample training set comprises (i) hybridization value(s) of the at least five biomarkers from a sample that overexpresses the at least five biomarkers, or overexpresses a subset of the at least five biomarkers, (ii) hybridization values of the at least five biomarkers from a reference proliferative sample, or (iii) hybridization values of the at least five biomarkers from a non-proliferative sample. A determination of whether the patient is likely to respond to angiogenesis inhibitor therapy, or a selection of the patient for angiogenesis inhibitor is then made based upon (i) the subject's proliferation signature or score alone or in combination with other characterization methods as described herein (e.g., AF-PRS, cancer subtype, immune subtype and/or TMB status) and (ii) the results of comparison.

TABLE 3
Biomarkers for hypoxia profile

| | | GenBank |
|---|---|---|
| Name | Abbreviation | Accession No. |
| RRAGD | Ras-related GTP binding D | BC003088 |
| FABP5 | fatty acid binding protein 5 | M94856 |
| UCHL1 | ubiquitin carboxyl-terminal esterase L1 | NM_004181 |
| GAL | Galanin | BC030241 |
| PLOD | procollagen-lysine, 2-oxoglutarate | M98252 |
| | 5-dioxygenase lysine hydroxylase | |
| DDIT4 | DNA-damage-inducible transcript 4 | NM_019058 |
| VEGF | vascular endothelial growth factor | M32977 |
| ADM | Adrenomedullin | NM_001124 |
| ANGPTL4 | angiopoietin-like 4 | AF202636 |
| NDRG1 | N-myc downstream regulated gene 1 | NM_006096 |
| NP | nucleoside phosphorylase | NM_000270 |
| SLC16A3 | solute carrier family 16 monocarboxylic | NM_004207 |
| | acid transporters, member 3 | |
| C14ORF58 | chromosome 14 open reading frame 58 | AK000378 |

The aforementioned set of thirteen biomarkers, or a subset thereof, is also referred to herein as a “hypoxia profile”.

In one embodiment, the method provided herein includes determining the levels of at least five biomarkers, at least six biomarkers, at least seven biomarkers, at least eight biomarkers, at least nine biomarkers, or at least ten biomarkers, or five to thirteen, six to thirteen, seven to thirteen, eight to thirteen, nine to thirteen or ten to thirteen biomarkers selected from RRAGD, FABP5, UCHL1, GAL, PLOD, DDIT4, VEGF, ADM, ANGPTL4, NDRG1, NP, SLC16A3, and C140RF58 in a sample obtained from a subject. Biomarker expression in some instances may be normalized against the expression levels of all RNA transcripts or their expression products in the sample, or against a reference set of RNA transcripts or their expression products. The reference set as explained throughout, may be an actual sample that is tested in parallel with the sample, or may be a reference set of values from a database or stored dataset. Levels of expression, in one embodiment, are reported in number of copies, relative fluorescence value or detected fluorescence value. The level of expression of the biomarkers of the hypoxia profile together with an anti-folate predictive response signature alone, a proliferation signature or score alone, an anti-folate predictive response signature in combination with a proliferation signature or score alone, or in combination with other characterization methods as described herein (e.g., cancer subtype, immune subtype and/or TMB status) as determined using the methods provided herein can be used in the methods described herein to determine whether a patient is likely to respond to angiogenesis inhibitor therapy.

In one embodiment, the levels of expression of the thirteen biomarkers (or subsets thereof, as described above, e.g., five or more, from about five to about 13), are normalized against the expression levels of all RNA transcripts or their non-natural cDNA expression products, or protein products in the sample, or of a reference set of RNA transcripts or a reference set of their non-natural cDNA expression products, or a reference set of their protein products in the sample.

In one embodiment, angiogenesis inhibitor treatments include, but are not limited to an integrin antagonist, a selectin antagonist, an adhesion molecule antagonist, an antagonist of intercellular adhesion molecule (ICAM)-1, ICAM-2, ICAM-3, platelet endothelial adhesion molecule (PCAM), vascular cell adhesion molecule (VCAM)), lymphocyte function-associated antigen 1 (LFA-1), a basic fibroblast growth factor antagonist, a vascular endothelial growth factor (VEGF) modulator, a platelet derived growth factor (PDGF) modulator (e.g., a PDGF antagonist).

In one embodiment of determining whether a subject is likely to respond to an integrin antagonist, the integrin antagonist is a small molecule integrin antagonist, for example, an antagonist described by Paolillo et al. (Mini Rev Med Chem, 2009, volume 12, pp. 1439-1446, incorporated by reference in its entirety), or a leukocyte adhesion-inducing cytokine or growth factor antagonist (e.g., tumor necrosis factor-α (TNF-α), interleukin-1β (IL-1β), monocyte chemotactic protein-1 (MCP-1) and a vascular endothelial growth factor (VEGF)), as described in U.S. Pat. No. 6,524,581, incorporated by reference in its entirety herein.

The methods provided herein are also useful for determining whether a subject is likely to respond to one or more of the following angiogenesis inhibitors: interferon gamma 10, interferon gamma 10 (Actimmune®) with pirfenidone, ACUHTR028, αVβ5, aminobenzoate potassium, amyloid P, ANG1122, ANG1170, ANG3062, ANG3281, ANG3298, ANG4011, anti-CTGF RNAi, Aplidin, Astragalus membranaceus extract with salvia and schisandra chinensis, atherosclerotic plaque blocker, Azol, AZX100, BB3, connective tissue growth factor antibody, CT140, danazol, Esbriet, EXC001, EXC002, EXC003, EXC004, EXC005, F647, FG3019, Fibrocorin, Follistatin, FT011, a galectin-3 inhibitor, GKT137831, GMCTO1, GMCT02, GRMD01, GRMD02, GRN510, Heberon Alfa R, interferon α-2β, ITMN520, JKB119, JKB121, JKB122, KRX168, LPA1 receptor antagonist, MGN4220, MIA2, microRNA 29a oligonucleotide, MMI0100, noscapine, PBI4050, PBI4419, PDGFR inhibitor, PF-06473871, PGN0052, Pirespa, Pirfenex, pirfenidone, plitidepsin, PRM151, Px102, PYN17, PYN22 with PYN17, Relivergen, rhPTX2 fusion protein, RXI109, secretin, STX100, TGF-β Inhibitor, transforming growth factor, β-receptor 2 oligonucleotide, VA999260, XV615 or a combination thereof.

In another embodiment, a method is provided for determining whether a subject is likely to respond to one or more endogenous angiogenesis inhibitors. In a further embodiment, the endogenous angiogenesis inhibitor is endostatin, a 20 kDa C-terminal fragment derived from type XVIII collagen, angiostatin (a 38 kDa fragment of plasmin), a member of the thrombospondin (TSP) family of proteins. In a further embodiment, the angiogenesis inhibitor is a TSP-1, TSP-2, TSP-3, TSP-4 and TSP-5. Methods for determining the likelihood of response to one or more of the following angiogenesis inhibitors are also provided a soluble VEGF receptor, e.g., soluble VEGFR-1 and neuropilin 1 (NPR1), angiopoietin-1, angiopoietin-2, vasostatin, calreticulin, platelet factor-4, a tissue inhibitor of metalloproteinase (TIMP) (e.g., TIMP1, TIMP2, TIMP3, TIMP4), cartilage-derived angiogenesis inhibitor (e.g., peptide troponin I and chrondomodulin I), a disintegrin and metalloproteinase with thrombospondin motif 1, an interferon (IFN), (e.g., IFN-α, IFN-β, IFN-γ), a chemokine, e.g., a chemokine having the C-X-C motif (e.g., CXCL10, also known as interferon gamma-induced protein 10 or small inducible cytokine B10), an interleukin cytokine (e.g., IL-4, IL-12, IL-18), prothrombin, antithrombin III fragment, prolactin, the protein encoded by the TNFSF15 gene, osteopontin, maspin, canstatin, proliferin-related protein.

In one embodiment, a method for determining the likelihood of response to one or more of the following angiogenesis inhibitors is provided is angiopoietin-1, angiopoietin-2, angiostatin, endostatin, vasostatin, thrombospondin, calreticulin, platelet factor-4, TIMP, CDAI, interferon α, interferon β, vascular endothelial growth factor inhibitor (VEGI) meth-1, meth-2, prolactin, VEGI, SPARC, osteopontin, maspin, canstatin, proliferin-related protein (PRP), restin, TSP-1, TSP-2, interferon gamma 10, ACUHTR028, αVβ5, aminobenzoate potassium, amyloid P, ANG1122, ANG1170, ANG3062, ANG3281, ANG3298, ANG4011, anti-CTGF RNAi, Aplidin, Astragalus membranaceus extract with salvia and schisandra chinensis, atherosclerotic plaque blocker, Azol, AZX100, BB3, connective tissue growth factor antibody, CT140, danazol, Esbriet, EXC001, EXC002, EXC003, EXC004, EXC005, F647, FG3019, Fibrocorin, Follistatin, FT011, a galectin-3 inhibitor, GKT137831, GMCTO1, GMCT02, GRMD01, GRMD02, GRN510, Heberon Alfa R, interferon α-2β, ITMN520, JKB119, JKB121, JKB122, KRX168, LPA1 receptor antagonist, MGN4220, MIA2, microRNA 29a oligonucleotide, MMI0100, noscapine, PBI4050, PBI4419, PDGFR inhibitor, PF-06473871, PGN0052, Pirespa, Pirfenex, pirfenidone, plitidepsin, PRM151, Px102, PYN17, PYN22 with PYN17, Relivergen, rhPTX2 fusion protein, RXI109, secretin, STX100, TGF-β Inhibitor, transforming growth factor, β-receptor 2 oligonucleotide, VA999260, XV615 or a combination thereof.

In yet another embodiment, the angiogenesis inhibitor can include pazopanib (Votrient), sunitinib (Sutent), sorafenib (Nexavar), axitinib (Inlyta), ponatinib (Iclusig), vandetanib (Caprelsa), cabozantinib (Cometrig), ramucirumab (Cyramza), regorafenib (Stivarga), ziv-aflibercept (Zaltrap), motesanib, or a combination thereof. In another embodiment, the angiogenesis inhibitor is a VEGF inhibitor. In a further embodiment, the VEGF inhibitor is axitinib, cabozantinib, aflibercept, brivanib, tivozanib, ramucirumab or motesanib. In yet a further embodiment, the angiogenesis inhibitor is motesanib.

In one embodiment, the methods provided herein relate to determining a subject's likelihood of response to an antagonist of a member of the platelet derived growth factor (PDGF) family, for example, a drug that inhibits, reduces or modulates the signaling and/or activity of PDGF-receptors (PDGFR). For example, the PDGF antagonist, in one embodiment, is an anti-PDGF aptamer, an anti-PDGF antibody or fragment thereof, an anti-PDGFR antibody or fragment thereof, or a small molecule antagonist. In one embodiment, the PDGF antagonist is an antagonist of the PDGFR-α or PDGFR-β. In one embodiment, the PDGF antagonist is the anti-PDGF-β aptamer E10030, sunitinib, axitinib, sorefenib, imatinib, imatinib mesylate, nintedanib, pazopanib HCl, ponatinib, MK-2461, dovitinib, pazopanib, crenolanib, PP-121, telatinib, imatinib, KRN 633, CP 673451, TSU-68, Ki8751, amuvatinib, tivozanib, masitinib, motesanib diphosphate, dovitinib dilactic acid, linifanib (ABT-869).

Upon making a determination of whether a patient is likely to respond to angiogenesis inhibitor therapy, or selecting a patient for angiogenesis inhibitor therapy, in one embodiment, the patient is administered the angiogenesis inhibitor. The angiogenesis in inhibitor can be any of the angiogenesis inhibitors described herein.

### Immunotherapy

In one embodiment, provided herein is a method for determining whether a cancer patient is likely to respond to immunotherapy by determining an anti-folate predictive response signature alone, a proliferation signature or score alone, an anti-folate predictive response signature in combination with a proliferation signature or score alone, or in combination with other characterization methods as described herein (e.g., cancer subtype, immune subtype and/or TMB status) from a sample obtained from the patient and, based on the anti-folate predictive response signature alone, the proliferation signature or score alone, the anti-folate predictive response signature in combination with the proliferation signature or score alone, or in combination with other characterization methods as described herein (e.g., cancer subtype, immune subtype and/or TMB status), assessing whether the patient is likely to respond to or may benefit from immunotherapy. In another embodiment, provided herein is a method of selecting a patient suffering from cancer for immunotherapy by determining an anti-folate predictive response signature alone, a proliferation signature or score alone, an anti-folate predictive response signature in combination with a proliferation signature or score alone, or in combination with other characterization methods as described herein (e.g., cancer subtype, immune subtype and/or TMB status) of a sample from the patient and, based on the anti-folate predictive response signature alone, the proliferation signature or score alone, the anti-folate predictive response signature in combination with the proliferation signature or score alone, or in combination with other characterization methods as described herein (e.g., cancer subtype, immune subtype and/or TMB status), selecting the patient for immunotherapy. The immunotherapy can be any immunotherapy provided herein. In one embodiment, the immunotherapy comprises administering one or more checkpoint inhibitors. The checkpoint inhibitors can be any checkpoint inhibitor or modulator provided herein such as, for example, a checkpoint inhibitor that targets or interacts with cytotoxic T-lymphocyte antigen 4 (CTLA4), programmed death 1 (PD-1) or its ligands (e.g., PD-L1), lymphocyte activation gene-3 (LAG3), B7 homolog 3 (B7-H3), B7 homolog 4 (B7-H4), indoleamine (2,3)-dioxygenase (IDO), adenosine A2a receptor, neuritin, B- and T-lymphocyte attenuator (BTLA), killer immunoglobulin-like receptors (KIR), T cell immunoglobulin and mucin domain-containing protein 3 (TIM-3), inducible T cell costimulator (ICOS), CD27, CD28, CD40, CD137, or combinations thereof.

In another embodiment, the immunotherapeutic agent is a checkpoint inhibitor. In some cases, a method for determining the likelihood of response to one or more checkpoint inhibitors is provided. In one embodiment, the checkpoint inhibitor is a PD-1/PD-LI checkpoint inhibitor. The PD-1/PD-LI checkpoint inhibitor can be nivolumab, pembrolizumab, atezolizumab, durvalumab, lambrolizumab, or avelumab. In one embodiment, the checkpoint inhibitor is a CTLA-4 checkpoint inhibitor. The CTLA-4 checkpoint inhibitor can be ipilimumab or tremelimumab. In one embodiment, the checkpoint inhibitor is a combination of checkpoint inhibitors such as, for example, a combination of one or more PD-1/PD-LI checkpoint inhibitors used in combination with one or more CTLA-4 checkpoint inhibitors.

In one embodiment, the immunotherapeutic agent is a monoclonal antibody. In some cases, a method for determining the likelihood of response to one or more monoclonal antibodies is provided. The monoclonal antibody can be directed against tumor cells or directed against tumor products. The monoclonal antibody can be panitumumab, matuzumab, necitumunab, trastuzumab, amatuximab, bevacizumab, ramucirumab, bavituximab, patritumab, rilotumumab, cetuximab, immu-132, or demcizumab.

In yet another embodiment, the immunotherapeutic agent is a therapeutic vaccine. In some cases, a method for determining the likelihood of response to one or more therapeutic vaccines is provided. The therapeutic vaccine can be a peptide or tumor cell vaccine. The vaccine can target MAGE-3 antigens, NY-ESO-1 antigens, p53 antigens, survivin antigens, or MUC1 antigens. The therapeutic cancer vaccine can be GVAX (GM-CSF gene-transfected tumor cell vaccine), belagenpumatucel-L (allogeneic tumor cell vaccine made with four irradiated NSCLC cell lines modified with TGF-beta2 antisense plasmid), MAGE-A3 vaccine (composed of MAGE-A3 protein and adjuvant AS15), (l)-BLP-25 anti-MUC-1 (targets MUC-1 expressed on tumor cells), CimaVax EGF (vaccine composed of human recombinant Epidermal Growth Factor (EGF) conjugated to a carrier protein), WT1 peptide vaccine (composed of four Wilms' tumor suppressor gene analogue peptides), CRS-207 (live-attenuated Listeria monocytogenes vector encoding human mesothelin), Bec2/BCG (induces anti-GD3 antibodies), GV1001 (targets the human telomerase reverse transcriptase), TG4010 (targets the MUC1 antigen), racotumomab (anti-idiotypic antibody which mimicks the NGcGM3 ganglioside that is expressed on multiple human cancers), tecemotide (liposomal BLP25; liposome-based vaccine made from tandem repeat region of MUC1) or DRibbles (a vaccine made from nine cancer antigens plus TLR adjuvants).

In one embodiment, the immunotherapeutic agent is a biological response modifier. In some cases, a method for determining the likelihood of response to one or more biological response modifiers is provided. The biological response modifier can trigger inflammation such as, for example, PF-3512676 (CpG 7909) (a toll-like receptor 9 agonist), CpG-ODN 2006 (downregulates Tregs), Bacillus Calmette-Guerin (BCG), mycobacterium vaccae (SRL172) (nonspecific immune stimulants now often tested as adjuvants). The biological response modifier can be cytokine therapy such as, for example, IL-2+ tumor necrosis factor alpha (TNF-alpha) or interferon alpha (induces T-cell proliferation), interferon gamma (induces tumor cell apoptosis), or Mda-7 (IL-24) (Mda-7/IL-24 induces tumor cell apoptosis and inhibits tumor angiogenesis). The biological response modifier can be a colony-stimulating factor such as, for example granulocyte colony-stimulating factor. The biological response modifier can be a multi-modal effector such as, for example, multi-target VEGFR: thalidomide and analogues such as lenalidomide and pomalidomide, cyclophosphamide, cyclosporine, denileukin diftitox, talactoferrin, trabecetedin or all-trans-retinmoic acid.

In one embodiment, the immunotherapy is cellular immunotherapy. In some cases, a method for determining the likelihood of response to one or more cellular therapeutic agents. The cellular immunotherapeutic agent can be dendritic cells (DCs) (ex vivo generated DC-vaccines loaded with tumor antigens), T-cells (ex vivo generated lymphokine-activated killer cells; cytokine-induce killer cells; activated T-cells; gamma delta T-cells), or natural killer cells.

### Radiotherapy

In one embodiment, provided herein is a method for determining whether a patient is likely to respond to radiotherapy by determining an anti-folate predictive response signature alone, a proliferation signature or score alone, an anti-folate predictive response signature in combination with a proliferation signature or score alone, or in combination with other characterization methods as described herein (e.g., cancer subtype, immune subtype and/or TMB status) of a sample obtained from the patient and, based on the anti-folate predictive response signature alone, the proliferation signature or score alone, the anti-folate predictive response signature in combination with the proliferation signature or score alone, or in combination with other characterization methods as described herein (e.g., cancer subtype, immune subtype and/or TMB status), assessing whether the patient is likely to respond to or benefit from radiotherapy. In another embodiment, provided herein is a method of selecting a patient suffering from cancer for radiotherapy by determining an anti-folate predictive response signature alone, a proliferation signature or score alone, an anti-folate predictive response signature in combination with a proliferation signature or score alone, or in combination with other characterization methods as described herein (e.g., cancer subtype, immune subtype and/or TMB status) of a sample from the patient and, based on the anti-folate predictive response signature alone, the proliferation signature or score alone, the anti-folate predictive response signature in combination with the proliferation signature or score alone, or in combination with other characterization methods as described herein (e.g., cancer subtype, immune subtype and/or TMB status), selecting the patient for radiotherapy.

In some embodiments, the radiotherapy can include but are not limited to proton therapy and external-beam radiation therapy. In some embodiments, the radiotherapy can include any types or forms of treatment that is suitable for patients with specific types of cancer. In some embodiments, the surgery can include laser technology, excision, dissection, and reconstructive surgery.

In some embodiments, a patient with a specific type of cancer can have or display resistance to radiotherapy. Radiotherapy resistance in any cancer of subtype thereof can be determined by measuring or detecting the expression levels of one or more genes known in the art and/or provided herein associated with or related to the presence of radiotherapy resistance. Genes associated with radiotherapy resistance can include NFE2L2, KEAP1 and CUL3. In some embodiments, radiotherapy resistance can be associated with the alterations of KEAP1 (Kelch-like ECH-associated protein 1)/NRF2 (nuclear factor E2-related factor 2) pathway. Association of a particular gene to radiotherapy resistance can be determined by examining expression of said gene in one or more patients known to be radiotherapy non-responders and comparing expression of said gene in one or more patients known to be radiotherapy responders.

### Surgical Intervention

In one embodiment, provided herein is a method for determining whether a cancer patient is likely to respond to surgical intervention by determining an anti-folate predictive response signature alone, a proliferation signature or score alone, an anti-folate predictive response signature in combination with a proliferation signature or score alone, or in combination with other characterization methods as described herein (e.g., cancer subtype, immune subtype and/or TMB status) of a sample obtained from the patient and, based on the anti-folate predictive response signature alone, the proliferation signature or score alone, the anti-folate predictive response signature in combination with the proliferation signature or score alone, or in combination with other characterization methods as described herein (e.g., cancer subtype, immune subtype and/or TMB status), assessing whether the patient is likely to respond to or benefit from surgery. In another embodiment, provided herein is a method of selecting a patient suffering from cancer for surgery by determining an anti-folate predictive response signature alone, a proliferation signature or score alone, an anti-folate predictive response signature in combination with a proliferation signature or score alone, or in combination with other characterization methods as described herein (e.g., cancer subtype, immune subtype and/or TMB status) of a sample from the patient and, based on the anti-folate predictive response signature alone, the proliferation signature or score alone, the anti-folate predictive response signature in combination with the proliferation signature or score alone, or in combination with other characterization methods as described herein (e.g., cancer subtype, immune subtype and/or TMB status), selecting the patient for surgery.

In some embodiments, surgery approaches for use herein can include but are not limited to minimally invasive or endoscopic head and neck surgery (eHNS), Transoral Robotic Surgery (TORS), Transoral Laser Microsurgery (TLM), Endoscopic Thyroid and Neck Surgery, Robotic Thyroidectomy, Minimally Invasive Video-Assisted Thyroidectomy (MIVAT), and Endoscopic Skull Base Tumor Surgery. In some embodiments, the surgery can include any types of surgical treatment that is suitable for cancer patients. In one embodiment, the suitable treatment is surgery.

### Detection Methods

In one embodiment, the methods and compositions provided herein allow for the detection of at least one nucleic acid or a plurality of biomarkers in a sample (e.g. tumor sample) obtained from a subject suffering from or suspected of suffering from a cancer. The at least one nucleic acid or plurality of classifier biomarkers can be a classifier biomarker or set of classifier biomarkers provided herein. In one embodiment, the at least one nucleic acid or plurality of classifier biomarkers detected using the methods and compositions provided herein are selected from Table 1 or Table 2. In one embodiment, the methods of detecting the nucleic acid(s) (e.g., classifier biomarkers) in the sample (e.g., tumor sample) obtained from the subject comprises, consists essentially of, or consists of measuring the expression level of at least one or a plurality of biomarkers using any of the methods provided herein. The biomarkers can be selected from Table 1 or Table 2. In some cases, the plurality of biomarker nucleic acids comprises, consists essentially of or consists of at least two biomarker nucleic acids, at least 8 biomarker nucleic acids, at least 16 biomarker nucleic acids, at least 24 biomarker nucleic acids, at least 32 biomarker nucleic acids, or all 48 biomarkers nucleic acids of Table 1. In some cases, the plurality of biomarker nucleic acids comprises, consists essentially of or consists of at least two biomarker nucleic acids, at least 2 biomarker nucleic acids, at least 4 biomarker nucleic acids, at least 6 biomarker nucleic acids, at least 8 biomarker nucleic acids, at least 10 biomarker nucleic acids, at least 12 biomarker nucleic acids, at least 14 biomarker nucleic acids, at least 16 biomarker nucleic acids, at least 18 biomarker nucleic acids, at least 20 biomarker nucleic acids, at least 22 biomarker nucleic acids, at least 24 biomarker nucleic acids, or all 26 biomarkers nucleic acids of Table 2. The detection can be by using any amplification, hybridization and/or sequencing assay disclosed herein.

In another embodiment, the methods and compositions provided herein allow for the detection of at least one nucleic acid or a plurality of nucleic acids in a sample (e.g. tumor sample) obtained from a subject suffering from or suspected of suffering from a cancer such that the at least one nucleic acid is or the plurality of nucleic acids are selected from the biomarkers listed in Table 1 and the detection of at least one biomarker or a plurality of biomarkers from a set of biomarkers whose presence, absence and/or level of expression is indicative of proliferation. The set of biomarkers for indicating proliferation can be the set of biomarkers listed in Table 2. The detection can be by using any amplification, hybridization and/or sequencing assay disclosed herein.

In another embodiment, the methods and compositions provided herein allow for the detection of at least one nucleic acid or a plurality of nucleic acids in a sample (e.g. tumor sample) obtained from a subject suffering from or suspected of suffering from a cancer such that the at least one nucleic acid is or the plurality of nucleic acids are selected from the biomarkers listed in Table 1 and the detection of at least one biomarker from a set of biomarkers whose presence, absence and/or level of expression is indicative of immune activation. The set of biomarkers for indicating immune activation can be gene expression signatures of Adaptive Immune Cells (ATC) and/or Innate Immune Cells (TTC) immune biomarkers, interferon genes, major histocompatibility complex, class II (MHC 11) genes or a combination thereof as described in WO 2017/201165. The gene expression signatures of both IIC and AIC can be any gene signatures known in the art such as, for example, the gene signature listed in Bindea et al. (Immunity 2013; 39(4); 782-795). The detection can be at the nucleic acid level. The detection can be by using any amplification, hybridization and/or sequencing assay disclosed herein.

In another embodiment, the methods and compositions provided herein allow for the detection of at least one nucleic acid or a plurality of nucleic acids in a sample (e.g. tumor sample) obtained from a subject suffering from or suspected of suffering from a cancer such that the at least one nucleic acid is or the plurality of nucleic acids are selected from the biomarkers listed in Table 2 and the detection of at least one biomarker from a set of biomarkers whose presence, absence and/or level of expression is indicative of immune activation. The set of biomarkers for indicating immune activation can be gene expression signatures of Adaptive Immune Cells (AIC) and/or Innate Immune Cells (IIC) immune biomarkers, interferon genes, major histocompatibiilty complex, class II (MHC II) genes or a combination thereof as described in WO 2017/201165. The gene expression signatures of both IIC and AIC can be any gene signatures known in the art such as, for example, the gene signature listed in Bindea et al. (Immunity 2013; 39(4); 782-795). The detection can be at the nucleic acid level. The detection can be by using any amplification, hybridization and/or sequencing assay disclosed herein.

### Kits

Kits for practicing the methods of the invention can be further provided. By “kit” can encompass any manufacture (e.g., a package or a container) comprising at least one reagent, e.g., an antibody, a nucleic acid probe or primer, etc., for specifically detecting the expression of a biomarker of the invention. The kit may be promoted, distributed, or sold as a unit for performing the methods of the present invention. Additionally, the kits may contain a package insert describing the kit and methods for its use.

In one embodiment, kits for practicing the methods of the invention are provided. Such kits are compatible with both manual and automated immunocytochemistry techniques (e.g., cell staining). These kits comprise at least one antibody directed to a biomarker of interest, chemicals for the detection of antibody binding to the biomarker, a counterstain, and, optionally, a bluing agent to facilitate identification of positive staining cells. Any chemicals that detect antigen-antibody binding may be used in the practice of the invention. The kits may comprise at least 2, at least 3, at least 4, at least 5, at least 6, at least 7, at least 8, at least 9, at least 10, or more antibodies for use in the methods of the invention.

### EXAMPLES

The present invention is further illustrated by reference to the following Examples. However, it should be noted that these Examples, like the embodiments described above, are illustrative and are not to be construed as restricting the scope of the invention in any way.

### Example 1—Development and Validation of a Proliferation Signature

### Objective

This example describes the generation of a gene signature for determining the presence of cell proliferation in a sample obtained from a subject suffering from or suspected of suffering from cancer. Overall, the goal of the studies in this example was to generate a single proliferation gene signature that can be used to assess the presence of cell proliferation across a broad group of tumor types. The use of this proliferation gene signature could be subsequently used to improve tumor classification that could inform prognosis, drug response and patient management based on underlying genomic and biologic tumor characteristics.

### Methods and Results

Data associated with the 2018 TCGA Pan-cancer publications (gdc.cancer.gov/about-data/publications/pancanatlas) was downloaded. In particular, the expression profile data from primary solid tumor samples that had expression data from the “EBPlusPlusAdjustPANCAN_IlluminaHiSeq_RNASeqV2” platform from the TCGA data set was used, while data from “do_not_use=False” specified in the sample quality file (merged_sample_quality_annotations.tsv) as well as data from samples from the pilot study (designated tumor type=“FFFP”) were excluded. There were n=8542 samples remaining from 30 tumor types. The 30 tumor types were kidney renal papillary cell carcinoma (KIRP); breast invasive carcinoma (BRCA); thyroid cancer (THCA); bladder urothelial carcinoma (BLCA); prostate adenocarcinoma (PRAD); kidney chromophobe (KICH); cervical squamous cell carcinoma and endocervical adenocarcinoma (CESC); kidney renal clear cell carcinoma (KIRC); liver hepatocellular carcinoma (LIHC); low grade glioma (LGG); sarcoma (SARC); lung adenocarcinoma (LUAD); colon adenocarcinoma (COAD); head and neck squamous cell carcinoma (HNSC); uterine corpus endometrial carcinoma (UCEC); glioblastoma multiforme (GBM); esophageal carcinoma (ESCA); stomach adenocarcinoma (STAD); ovarian serous cystadenocarcinoma (OV); rectum adenocarcinoma (READ); adrenocortical carcinoma (ACC); uveal melanoma (UVM); mesothelioma (MESO); pheochromocytoma and paraganglioma (PCPG); skin cutaneous melanoma (SKCM); uterine carcinsarcoma (UCS); lung squamous cell carcinoma (LUSC); testicular germ cell tumors (TGCT); cholangiocarcinoma (CHOL); pancreatic adenocarcinoma (PAAD). It should be noted that the 8542 samples, according to TCGA barcode, were from primary solid tumors.

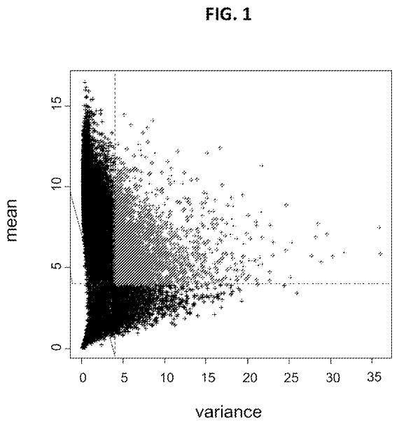

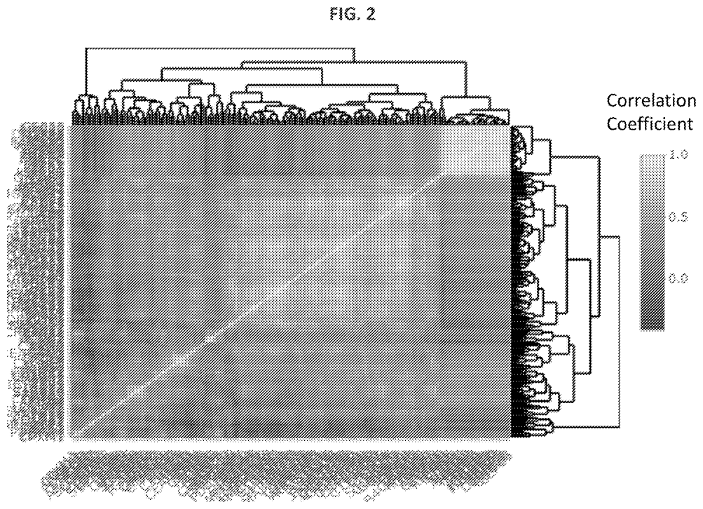

The resulting RNAseq expression data from the 8542 samples was then used to generate a pan-cancer proliferation gene signature. In summary, the samples were divided into a training set (⅔ of the data set; n=5694 samples) and test set (⅓ of the data set; n=2848 samples), balancing for uniform tumor type distributions. Gene expression values were log 2 transformed. Using the training set, genes with low variance and/or low mean were filtered out, while genes with mean variance and mean expression values greater than 4 were kept resulting in gene expression data for 2175 genes (see FIG. 1). Agglomerative hierarchical clustering with average linkage and correlation for distance was then performed. The resulting clustering dendrogram (see FIG. 2) was inspected for sub-clusters having extreme gene-gene correlation coefficients and harboring well-known proliferation genes, including MK167, BUB1, RRM2, and MYBL2, and found a set of 26 genes as shown in Table 2.

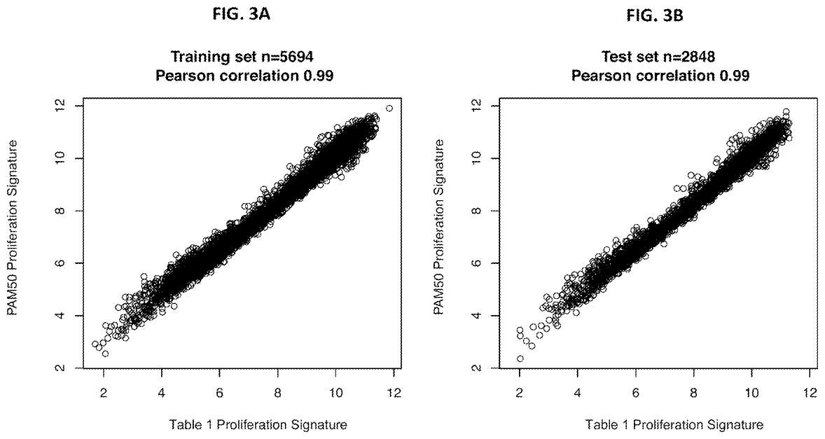

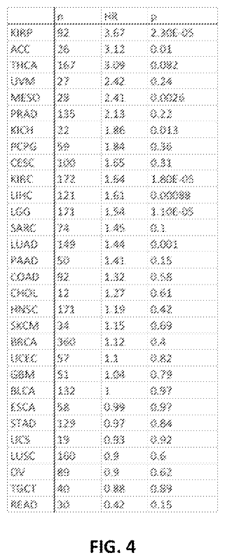

To validate the signature, the Table 2 proliferation signature (i.e., nucleic acid expression levels of the 26 classifier gene set) was determined for each sample for the TCGA data reserved as a test set as described above as well as the training set and the determined proliferation signatures for each sample in the test set and training set were converted to proliferation scores for each sample by calculating the mean gene expression across the Table 2 proliferation signature in each sample. In parallel, the 11-gene PAM50 proliferation signature described in Nielsen, Torsten O., Joel S. Parker, Samuel Leung, David Voduc, Mark Ebbert, Tammi Vickery, Sherri R. Davies et al. “A comparison of PAM50 intrinsic subtyping with immunohistochemistry and clinical prognostic factors in tamoxifen-treated estrogen receptor-positive breast cancer.” Clinical cancer research (2010): 1078-0432, which is hereby incorporated by reference in its entirety, was used on the same TCGA data reserved as a test set and training set to determine the proliferation score for each sample in the test set and training set using the PAM50 proliferation signature. In summary, whether using the Table 2 proliferation signature or the PAM50 proliferation signature, the proliferation scores determined therefrom were computed as the average log 2 transformed nucleic acid expression data across all genes in each respective signature. Subsequently, the Table 2 and PAM50 proliferation scores were examined for the presence of a correlation between the two proliferation scores for each sample from either the training set (see FIG. 3A) or test set (see FIG. 3B) using a Pearson correlation analysis. There was an overall correlation coefficient of 0.991 in the test set (FIG. 3B) and within tumor type correlation coefficients ranging from 0.872-0.994 between the two proliferation signatures in the test set (FIG. 3B). Similar correlation coefficients between the two signatures were also found when performing the same analysis on the training set described herein (see FIG. 3A).

### Example 2—Examination of the Use of the Proliferation Signature as a Prognostic Indicator

### Objective

This example describes the examination of the proliferation gene signature developed in Example 1 and found in Table 2 as a prognostic indicator for overall survival for determining the presence of cell proliferation in a sample obtained from a subject suffering from or suspected of suffering from cancer. Overall, the goal of the studies in this example was to determine if the proliferation signature has prognostic value across a myriad of tumor types.

### Methods and Results

In order to determine if the proliferation gene signature of Table 2 has prognostic utility, associations between overall survival and the proliferation signature were examined. Associations between overall survival and the proliferation signature were examined by fitting cox models with overall survival as the outcome and proliferation score as the predictor, reporting hazard ratios for proliferation score, and testing (Wald's test) whether the coefficient for proliferation score was different from zero. Proliferation score was left continuous in the model such that the interpretation of the hazard ratio values was mostly for direction of effect and not effect size (i.e., considering direction only, when the hazard ratio is greater than 1 then higher proliferation is associated with worse survival).

In the overall test set (n=2827), the Table 2 proliferation signature was prognostic (hazard ratio (HR)=1.37, p-value=3.3e-16, adjusted for tumor type using a stratified cox model). Associations were also examined separately in each tumor type, and cox model HR estimates varied between 0.42 and 3.67. As shown in FIG. 4, proliferation was significantly associated with worse overall survival in 8 (i.e., LUAD, LGG, LIHC, KIRC, KICH, MESO, ACC and KIRP) of the 30 tumor types (HR between 1.44 and 3.67 and p-value between 1.3e-02 and 1.1e-05, without multiplicity adjustment). HRs that were less than 1 were not significant.

### Example 3—Examination on the Use of Proliferation Signature as a Prognostic Indicator in Multiple Myeloma (MM)

### Objective

This example describes the examination of the proliferation gene signature developed in Example 1 and found in Table 2 as a prognostic indicator for disease specific survival. In this example the disease is multiple myeloma (MM).

### Methods and Results

In order to determine in specific subtypes of multiple myeloma (MM) had more or less proliferation, the proliferation score for samples from specific MM subtypes was determined using the proliferation signature of Table 2. The proliferation score and intrinsic subtypes of MM were determined using clinical data and normalized Affymetrix expression data generated from CD-138-selected plasma cells from bone marrow for n=414 newly diagnosed MM patients corresponding to Zhan F, et al. (2006) “The molecular classification of myeloma.” Blood 108: 2020-8 (incorporated herein by reference) was downloaded from GEO (GSE4581). In particular, the expression profiles were used determine the proliferation score using the signature of Table 2 and to assign patients to intrinsic gene-expression based subtypes (I-VII) as described in Chapman M A, et al. (2011) “Initial genome sequencing and analysis of multiple myeloma.” Nature 2011 Mar. 24; 471(7339):467-72 (incorporated herein by reference).

In order to determine if the proliferation score calculated using the proliferation signature of Table 2 has prognostic utility, associations between MM survival and the proliferation score were examined using a Kaplan-Meier plot.

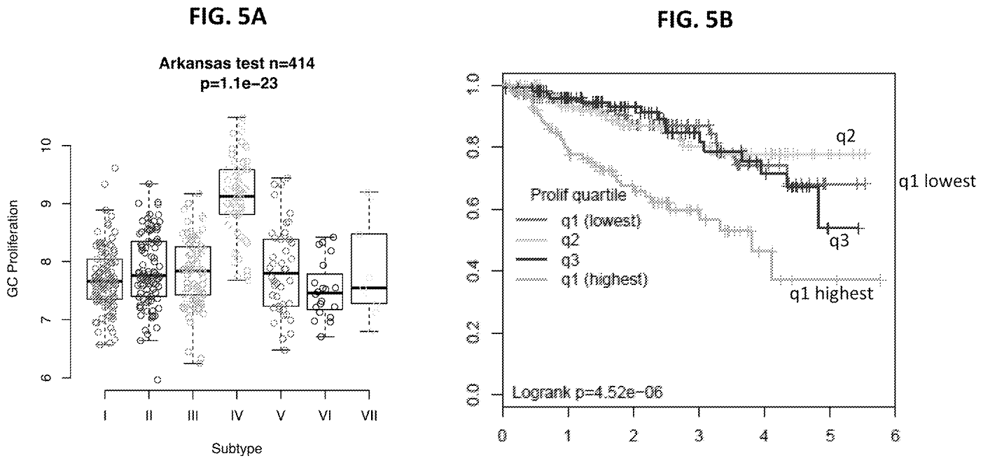

In the overall test set (n=414), the Table 2 proliferation score was found to be the highest for the MM intrinsic subtype IV (see FIG. 5A). Further, as shown in the Kaplan-Meier plot in FIG. 5B, when patients were grouped by proliferation quartiles, proliferation was significantly associated with worse survival.

### Example 4—Examination on the Use of a Lung Adenocarcinoma Subtyper as a Potential Antifolate Predictive Response Test for Lung Cancer

### Objective

Pemetrexed (LY231514) is a Lilly lung cancer drug in the folate analog inhibitor family. Other drugs in this family include Methotrexate, Trimetrexate, Lometrexol, Raltitrexed and Nolatrexed. Cells are dependent on a full supply of reduced folate to drive a series of 1-carbon reactions that result in synthesis of thymidylate and purines. Antifolates inhibit several enzymes that require this cofactor including synthesis, storage, and transport proteins and have been used in cancer therapy for over 50 years. Alimta (pemetrexed) is approved for first line treatment of patients with locally advanced or metastatic non-squamous NSCLC in combination with cisplatin. It is also approved with cisplatin for treatment of mesothelioma who are not surgery candidates. Mechanistically, Pemetrexed is a multifunctional inhibitor of pathways using folate and its inhibition on multiple targets has been considered a strength of the drug for cancer treatment as compared to other antifolates. It appears to be highly sensitive to thymidylate synthase levels and higher expression levels can inhibit the drug, which suggest the drug may be more sensitive to cells with decreased levels of these enzymes. While Alimta (pemetrexed) has been approved as a first line treatment of patients with locally advanced or metastatic non-squamous NSCLC in combination with cisplatin as well as patients with metastatic non-squamous NSCLC in combination with platinum chemotherapy and pembrolizumab and is also approved with cisplatin for treatment of mesothelioma in patients who are not surgery candidates, there appear to be subpopulations of patients within the approved treatment populations that respond better to antifolate treatment than others.

Additionally, the bronchioid subtype of LUAD (Group 1 from Fennel et al 2014) was previously shown to be more sensitive to pemetrexed by Fennel et al 2014 (Fennel et al., Association between Gene Expression Profiles and Clinical Outcome of Pemetrexed-Based Treatment in Patients with Advanced Non-Squamous Non-Small Cell Lung Cancer: Exploratory Results from a Phase II Study. PLOS One, 2014 (PMID: 25250715)). Moreover, overexpression of thymidylate synthetase (TYMS) has been shown to lead to a reduced sensitivity to this antifolate (e.g., pemetrexed) treatment (see Giovannetti E, Mey V, Nannizzi S, Pasqualetti G, Marini L, Del Tacca M and Danesi R: Cellular and pharmacogenetics foundation of synergistic interaction of pemetrexed and gemcitabine in human non-small-cell lung cancer cells. Mol Pharmacol 68: 110-118, 2005; Takezawa K, et al., Thymidylate synthase as a determinant of pemetrexed sensitivity in non-small cell lung cancer. Br J Cancer 104: 1594-1601, 2011 and Ozasa H et al., Significance of thymidylate synthase for resistance to pemetrexed in lung cancer. Cancer Sci 101: 161-166, 2010). Furthermore, lower expression/activity levels of pemetrexed drug targets such as thymidylate synthetase (TYMS) gene, dihydrofolate reductase (DHFR) gene, phosphoribosylglycinamide formyltransferase (GART) gene, 5-aminoimidazole-4-carboxamide ribonucleotide formyltransferase/IMP cyclohydrolase (ATIC) gene, methylenetetrahydrofolate dehydrogenase (NADP+ dependent) 1 like (MTHFD1L) gene may make tumors more sensitive to this antifolate (e.g., pemetrexed) treatment (see Liu, Qingyun et al. ‘Prognostic and Predictive Significance of Thymidylate Synthase Protein Expression in Non-Small Cell Lung Cancer: A Systematic Review and Meta-analysis’. 1 Jan. 2015: 65-78).

Accordingly, the purpose of this Example is to determine if a gene expression signature for subtyping lung adenocarcinoma has utility as an antifolate predictive response test. for specific cancer types.

### Methods

In order to determine if the lung adenocarcinoma (LUAD) subtyper of WO 2017/201165 (incorporated herein by reference) has utility as an antifolate predictive response signature (i.e., whether or not specific intrinsic subtypes of LUAD had more or less sensitivity to anti-folates (e.g., pemetrexed)), the expression levels of known pemetrexed targets (i.e., DHFR, TYMS, ATIC, MTHFD1L and GART genes) from specific LUAD subtypes were determined using RNA expression data from TCGA as described herein. Additionally, the proliferation score of each of these intrinsic LUAD subtypes was determined in order to examine how proliferation tracked across said subtypes and in comparison to the known pemetrexed drug targets.

The intrinsic subtypes of LUAD were determined using RNA-seq expression data from the TCGA LUAD (n=515) dataset, which was downloaded from Firehose (gdac.broadinstitute.org/). As indicated, the LUAD intrinsic bronchioid, magnoid and squamoid subtypes were determined by applying the 48-gene LUAD subtyper found in Table 1 of WO 2017/201165 to the TCGA LUAD dataset as described in WO2017/201165. In addition, the proliferations score for each sample from said dataset was determined using the 26-gene proliferation signature provided in Table 2 herein. Table 1 of WO 2017/201165 is re-created as Table 4 herein in order to show the gene centroids for the LUAD intrinsic subtypes.

TABLE 4
Gene Centroids of 48 Classifier Biomarkers for the Lung Adenocarcinoma
(AD) Subtypes (recreated from Table 1 of_WO2017/201165).

| | | | Bronchioid | Magnoid | Squamoid | |
|---|---|---|---|---|---|---|
| | | | (Terminal | (Proximal | (Proximal | GenBank |
| | | | Respiratory | Proliferative | Inflammatory | Acession |
| Gene # | Gene Symbol | Gene Name | Unit (TRU)) | (PP)) | (PI)) | Number* |
| 1 | FIGF | C-fos-induced | 2.129901586 | −1.173222174 | −1.545843019 | AY874421.1 |
| | | growth factor | | | | |
| 2 | CTSH | Cathepsin H | 1.099895637 | −0.797376345 | −0.531006607 | NM_004390.4 |
| 3 | SCTR | Secretin receptor | 2.043898366 | −1.911062476 | −1.836386831 | NM_002980.2 |
| 4 | CYP4B1 | Cytochrome | 2.462733828 | −1.447070454 | −1.481195844 | NM_001319161.1 |
| | | P450 family | | | | |
| | | 4 subfamily | | | | |
| | | B member 1 | | | | |
| 5 | GPR116 | G protein- | 1.289460077 | −0.972597916 | −0.731487829 | AY140958.1 |
| | | coupled | | | | |
| | | receptor 116 | | | | |
| 6 | ADH1B | Alcohol | 2.013525076 | −1.580299515 | −1.094580574 | NM_001286650.1 |
| | | dehydrogenase | | | | |
| | | 1B (class I) | | | | |
| 7 | CBX7 | Chromobox 7 | 0.728027298 | −0.698222051 | −0.243583657 | NM_175709.3 |
| 8 | HLF | Hepatic | 1.479193357 | −1.28826965 | −1.018563422 | M95585.1 |
| | | leukemia factor | | | | |
| 9 | CEP55 | Centrosomal | −1.524932169 | 0.5743319 | 0.580921528 | NM_018131.4 |
| | | protein 55 | | | | |
| 10 | TPX2 | Tpx2, | −1.704080763 | 0.587761579 | 0.583674937 | NM_012112.4 |
| | | Microtubule- | | | | |
| | | associated | | | | |
| 11 | BUB1B | BUB1 mitotic | −1.531514951 | 0.769199954 | 0.543731288 | AF107297.1 |
| | | checkpoint | | | | |
| | | serine/threonine | | | | |
| | | kinase B | | | | |
| 12 | KIF4A | Kinesin family | −1.794045266 | 0.570328759 | 0.599399471 | NM_012310.4 |
| | | member 4A | | | | |
| 13 | CCNB2 | Cyclin B2 | −1.442466223 | 0.602807712 | 0.526093335 | NM_004701.3 |
| 14 | KIF14 | Kinesin family | −1.66445145 | 0.762295222 | 0.543132477 | NM_014875.2 |
| | | member 14 | | | | |
| 15 | MELK | Maternal | −1.685012297 | 0.584181432 | 0.694064307 | NM_014791.3 |
| | | embryonic leucine | | | | |
| | | zipper kinase | | | | |
| 16 | KIF11 | Kinesin family | −1.183768087 | 0.693181955 | 0.481955763 | NM_004523.3 |
| | | member 11 | | | | |
| 17 | FGL1 | Fibrinogen | −0.978882607 | 4.89751413 | −1.958269455 | NM_004467.3 |
| | | like 1 | | | | |
| 18 | PBK | PDZ binding | −1.407694417 | 1.278522857 | 0.404652088 | NM_018492.3 |
| | | kinase | | | | |
| 19 | HSPD1 | Heat shock | −0.469703958 | 0.624572377 | 0.111400174 | NM_002156.4 |
| | | protein family D | | | | |
| | | (Hsp60) member 1 | | | | |
| 20 | TDG | Thymine DNA | −0.351189471 | 0.60348929 | 0.076442589 | NM_003211.4 |
| | | glycosylase | | | | |
| 21 | PRC1 | Protein regulator | −1.159074285 | 0.797575854 | 0.461100041 | NM_003981.3 |
| | | of cytokinesis 1 | | | | |
| 22 | DUSP4 | Dual specificity | −0.704273045 | 1.933259798 | −0.283343923 | NM_001394.6 |
| | | phosphatase 4 | | | | |
| 23 | GTPBP4 | GTP binding | −0.467281005 | 0.543583167 | 0.038904486 | NM_012341.2 |
| | | protein 4 | | | | |
| 24 | ZWINT | ZW10 interacting | −1.062801846 | 0.741405035 | 0.418738839 | NM_007057.3 |
| | | kinetochore protein | | | | |
| 25 | TLR2 | Toll like receptor 2 | 0.672774085 | −1.389004155 | 0.098176794 | NM_001318787.1 |
| 26 | CD74 | CD74 molecule | 0.689011729 | −1.365243826 | 0.239872217 | NM_001025159.2 |
| 27 | HLA-DPB1 | Major | 0.70548523 | −1.431001558 | 0.157288388 | M83664.1 |
| | | histocompatibility | | | | |
| | | complex, class | | | | |
| | | II, DP beta 1 | | | | |
| 28 | HLA-DPA1 | Major | 0.620746458 | −1.622212879 | 0.206805676 | NM_033554.3 |
| | | histocompatibility | | | | |
| | | complex, class | | | | |
| | | II, DP alpha 1 | | | | |
| 29 | HLA-DRA | Major | 0.47615106 | −1.517000712 | 0.209882138 | NM_019111.4 |
| | | histocompatibility | | | | |
| | | complex, class | | | | |
| | | II, DR alpha | | | | |
| 30 | ITGB2 | Integrin | 0.227015125 | −1.489015066 | 0.473986644 | NM_000211.4 |
| | | subunit beta 2 | | | | |
| 31 | FAS | Fas cell surface | 0.120924174 | −1.244937359 | 0.608312102 | KM114217.1 |
| | | death receptor | | | | |
| 32 | HLA-DRB1 | Major | 0.561088415 | −1.639812592 | 0.272965507 | NM_002124.3 |
| | | histocompatibility | | | | |
| | | complex, class | | | | |
| | | II, DR beta 1 | | | | |
| 33 | PLAU | Plasminogen | −0.723116671 | −0.71054832 | 1.628730403 | NM_002658.4 |
| | | activator, | | | | |
| | | urokinase | | | | |
| 34 | GBP1 | Guanylate | −0.302372654 | −0.688857626 | 1.204326606 | NM_002053.2 |
| | | binding | | | | |
| | | protein 1 | | | | |
| 35 | DSE | Dermatan | −0.101374419 | −0.602077696 | 0.748133278 | NM_013352.3 |
| | | sulfate | | | | |
| | | epimerase | | | | |
| 36 | CCDC109B | Coiled-coil | −0.13855818 | −0.703783616 | 0.7964386 | BC002633.2 |
| | | domain | | | | |
| | | containing | | | | |
| | | 109B | | | | |
| 37 | TGFBI | Transforming | −0.328357044 | −0.746331889 | 1.164873128 | NM_000358.2 |
| | | growth factor | | | | |
| | | beta induced | | | | |
| 38 | CXCL10 | C-X-C motif | −0.434345777 | −0.62067894 | 1.70756508 | NM_001565.3 |
| | | chemokine | | | | |
| | | ligand 10 | | | | |
| 39 | LGALS1 | Lectin, | −0.291230377 | −0.549722715 | 0.957730776 | NM_002305.3 |
| | | galactoside | | | | |
| | | binding | | | | |
| | | soluble 1 | | | | |
| 40 | TUBB6 | Tubulin beta | −0.153163739 | −0.328431543 | 0.781293298 | NM_032525.2 |
| | | 6 class V | | | | |
| 41 | GJB1 | Gap junction | 1.567852415 | 0.672938467 | −3.61601989 | NM_001097642.2 |
| | | protein beta 1 | | | | |
| 42 | RAP1GAP | RAP1 GTPase | 1.019990653 | 0.138302482 | −1.426817837 | NM_001145658.1 |
| | | activating | | | | |
| | | protein | | | | |
| 43 | CACNA2D2 | Calcium voltage- | 1.610819757 | −0.126189977 | −2.357279793 | NM_001005505.2 |
| | | gated channel | | | | |
| | | auxiliary subunit | | | | |
| | | alpha2delta2 | | | | |
| 44 | SELENBP1 | Selenium | 1.0475958 | −0.331350331 | −1.209058454 | NM_003944.3 |
| | | binding | | | | |
| | | protein 1 | | | | |
| 45 | TFCP2L1 | Transcription | 0.218606218 | 0.952552471 | −1.320932951 | NM_014553.2 |
| | | factor | | | | |
| | | CP2-like 1 | | | | |
| 46 | SORBS2 | Sorbin and | 0.603086366 | 0.462888705 | −1.412139816 | NM_001270771.1 |
| | | SH3 domain | | | | |
| | | containing 2 | | | | |
| 47 | UNC13B | Unc-13 | 0.293706669 | 0.418115853 | −0.978505828 | NM_006377.3 |
| | | homolog B | | | | |
| 48 | TACC2 | Transforming | 0.206302979 | 0.928437713 | −0.822332116 | AF220152.2 |
| | | acidic coiled- | | | | |
| | | coil containing | | | | |
| | | protein 2 | | | | |

*Each GenBank Accession Number is a representative or exemplary GenBank Accession Number for the listed gene and is herein incorporated by reference in its entirety for all purposes. Further, each listed representative or exemplary accession number should not be construed to limit the claims to the specific accession number.

Additionally, the tumor mutational burden (TMB) was assessed for each of the LUAD subtypes using the nonsilent mutation burden per megabase data available in the supplementary TCGA information as described in Faruki et al., Lung Adenocarcinoma and Squamous Cell Carcinoma Gene Expression Subtypes Demonstrate Significant Differences in Tumor Immune Landscape; Journal of Thoracic Oncology; Volume 12, Issue 6, June 2017, Pages 943-953, which is incorporated by reference in its entirety for all purposes.

In order to determine if the AF-PRS of Table 1 has prognostic utility, survival differences between AF-PRS (+) and AF-PRS (−) samples were assessed using stratified cox models and Kaplan Meier plots in the TCGA non-small cell lung cancer (NSCLC) lung adenocarcinoma (LUAD) dataset which included and n of 506 with overall survival data. The TCGA data was collected prior to approval of anti-PD-L1 therapies in NSCLC and the standard of care was pemetrexed plus platinum.

### Results and Conclusions

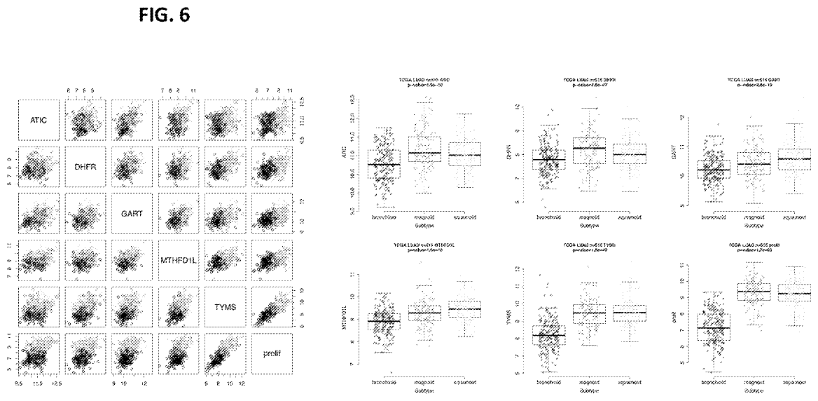

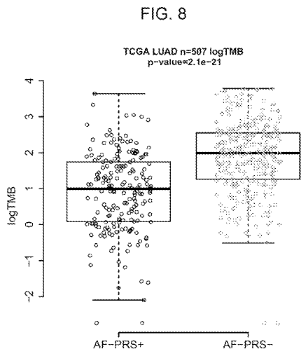

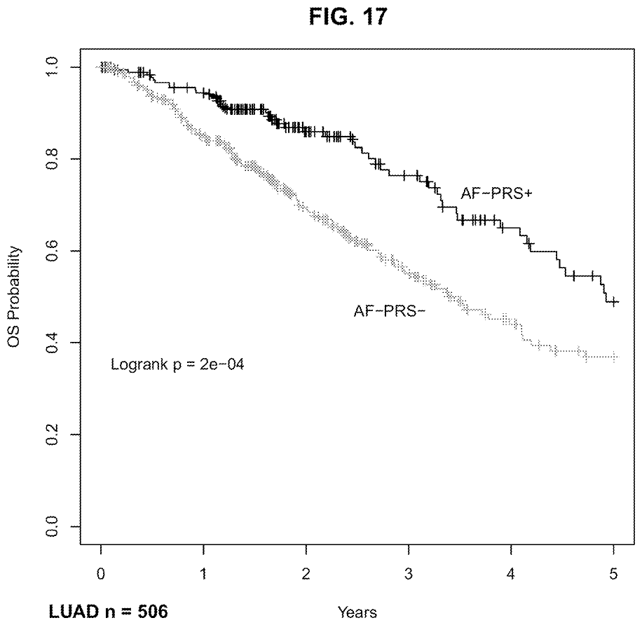

In the LUAD dataset (n=515), the Table 2 proliferation score was found to be the lowest for the LUAD bronchioid subtype (see FIG. 6). The LUAD bronchioid subtype also showed lower levels of expression of the key pemetrexed targets, DHFR, GART, ATIC, MTHFD1L and TYMS. Moreover, the TMB appears to be lowest in the bronchioid subtype (see FIG. 8). Overall, these results suggest that the bronchioid subtype of LUAD may be particularly susceptible to treatment with anti-folates such as pemetrexed vs. other LUAD subtypes, given that evidence exists that lower expression/activity levels of pemetrexed drug targets are more sensitive to this drugs treatment. Moreover, it seems that proliferation as detecting using the proliferation signature of Table 2 also tracked with the expression levels of the pemetrexed drug targets such that lower expression of these drug targets appears to occur in patients that also show lower proliferation (see. FIG. 6). Additionally, as shown in FIG. 17, there was a significantly longer median overall survival (OS) in the AF-PRS (+) group versus the AF-PRS (−) group (i.e., 4.9 years vs. 3.4 years).

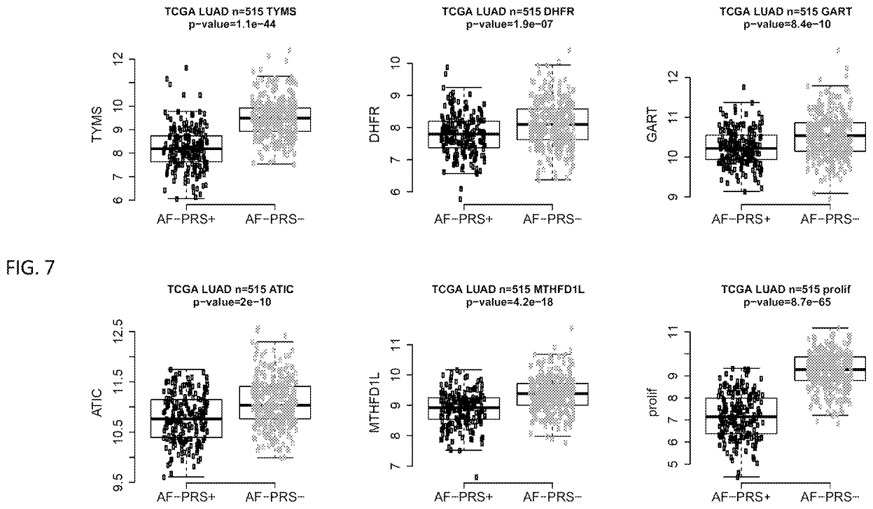

Based on the data, it seems that the results from FIG. 6 can be regrouped such that the bronchioid subtype of LUAD can be classified as one group (i.e., anti-folate predictive response signature (AF-PRS)+), while the other subtypes of LUAD (i.e., magnoid and squamoid) can be reclassified as a second group (i.e., AF-PRS −) as shown in FIG. 7. As such, analysis of LUAD patient gene expression data using the 48-gene LUAD subtyper of WO2017/201165 can be used as an antifolate predictive response signature (AF-PRS) for grouping patients as being AF-PRS (+) and thus likely to be responsive to anti-folate treatment or AF-PRS(−) and thus unlikely to respond to antifolate treatment. This AF-PRS can be used alone to assess antifolate predictive response or can be used in conjunction with or as an adjunct to assessing proliferation and/or expression levels of known pemetrexed drug targets.

### Example 5—Examination on the Use of a Lung Adenocarcinoma Subtyper as a Potential Antifolate Predictive Response Test Across Other Cancers

### Objective

As a follow-up to the results from Example 4 provided herein, the objective of this Example is to ascertain how known pemetrexed drug targets track across subtypes in other cancers and whether or not the 48-gene LUAD subtyper from WO2017/201165 can serve as an antifolate predictive response signature (AF-PRS) in cancer types other than lung cancer. Another objective of this Example is to examine how proliferation tracks with expression of the known pemetrexed drug targets and whether or not it mimics what was observed in LUAD.

### Methods

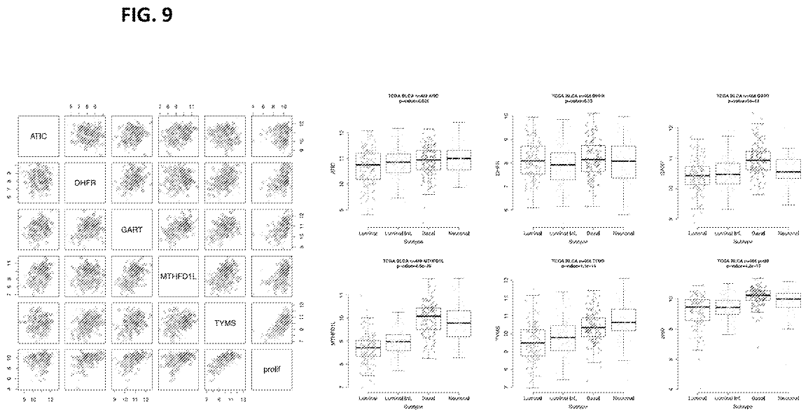

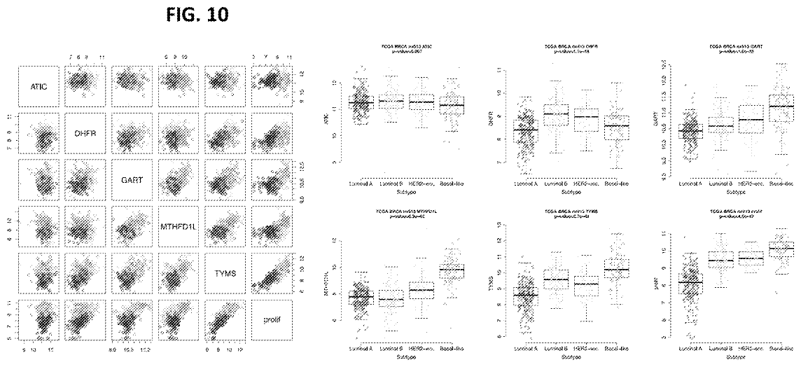

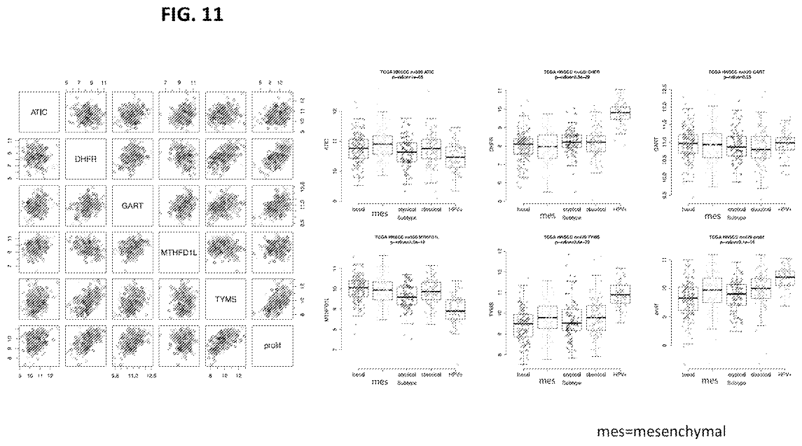

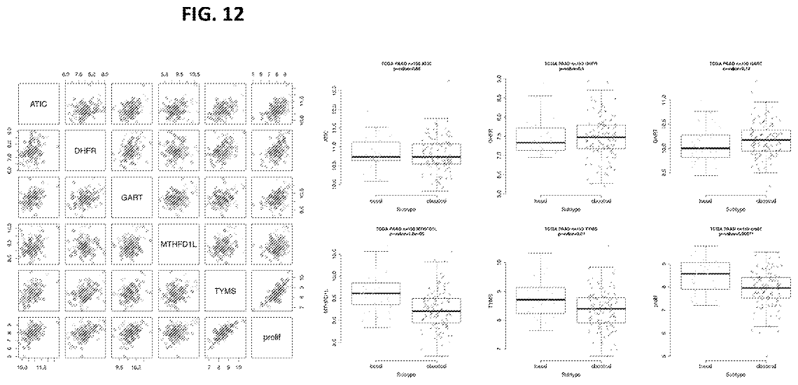

In an initial experiment, an analysis of the expression level of each of the pemetrexed drug target genes from Example 4 (i.e., DHFR, GART, ATIC, MTHFD1L and TYMS) and the proliferation gene signature found in Table 2 were examined across intrinsic subtypes of bladder cancer (BLCA; see FIG. 9), breast cancer (BRCA; see FIG. 10), head and neck squamous cell carcinoma (HNSCC; see FIG. 11) and pancreatic adenocarcinoma (PAAD; see FIG. 12). The intrinsic subtypes of BLCA were determined using the 60 gene signature or classifier biomarker set subtyper found in Table 5 below as recreated from Table 1 in PCT/US2019/017799 (which is herein incorporated by reference) using the dataset and analysis as described in PCT/US2019/017799. The intrinsic subtypes of HNSCC were determined using the 144 gene signature found in Table 6 below as recreated from Table 1 in PCT/US2018/045522 (which is herein incorporated by reference) using the dataset and analysis as described in PCT/US2018/045522. The intrinsic subtypes of PAAD were determined using the subtyper gene signature found in Table 7 below as recreated from Table 9 in US2017/0233827 (which is herein incorporated by reference) using the dataset and analysis as described in US2017/0233827. The intrinsic subtypes of BRCA were determined using the PAM50 subtyper gene signature found in Parker et al., J Clin Oncol. 2009 Mar. 10; 27(8): 1160-1167 and U.S. Pat. No. 9,631,239 (each of which is herein incorporated by reference) using the dataset and analysis as described in U.S. Pat. No. 9,631,239. The proliferation score and analysis of expression of the five (5) pemetrexed drug targets were done as discussed in Example 4.

Following this initial set of experiments, the 48-gene LUAD subtyper of Table 4 was applied to each of the datasets utilized for BLCA, BRCA, HNSCC and PAAD from the initial set of experiments using the methods described in WO2017/201165. Additionally, the 48-gene LUAD subtyper of Table 4 was applied to the TCGA squamous cell carcinoma (LUSC) dataset (n=501) using the methods described in WO2017/201165. Subsequently, the expression of the five (5) drug targets described above as well as proliferation was also examined in the subpopulations of BLCA, BRCA, HNSCC, PAAD and LUSC that classified as either bronchioid, magnoid and squamoid.

### Results and Conclusions

In the initial set of experiments, the luminal subtype of BLCA, the luminal A subtype of BRCA, the basal subtype of HNSCC and the classical subtype of PAAD all showed lower levels of expression for the pemetrexed drug targets and lower proliferation (see FIGS. 9-12) much like the bronchioid subtype of LUAD as shown in Example 4. As such, one would predict that each of these subtypes from these other cancers could be subtype populations that would also respond to antifolate activity.

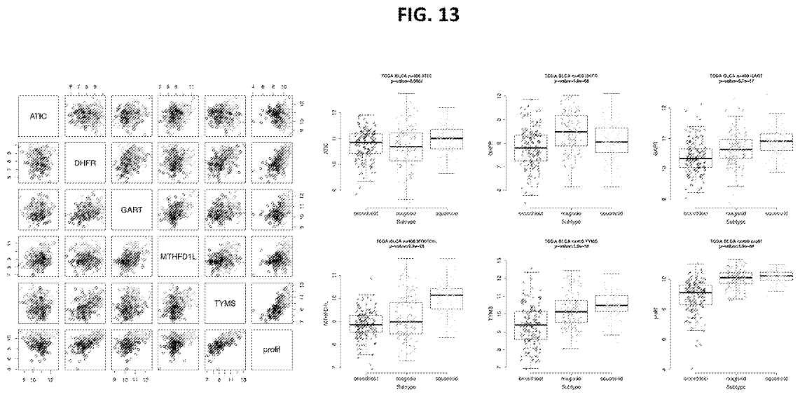

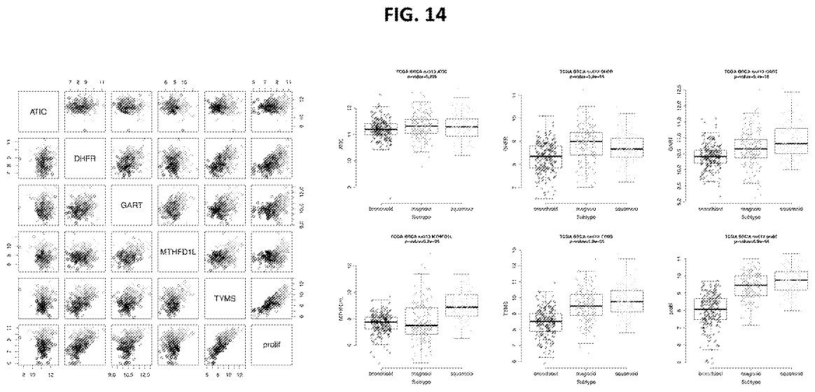

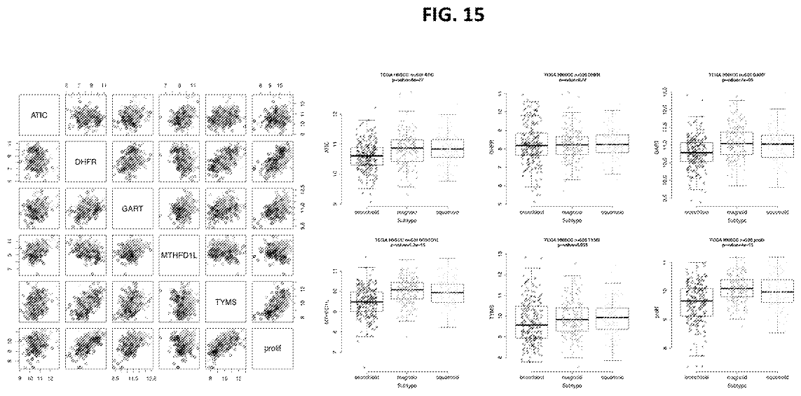

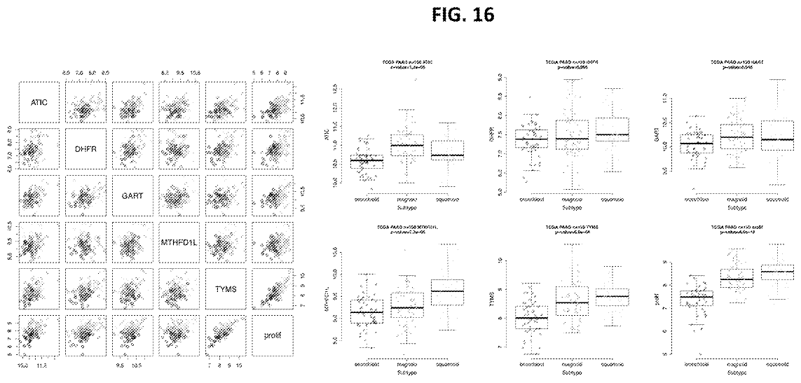

Interestingly, when the lung adenocarcinoma subtyper or signature (i.e., Table 4) was applied to the datasets from the BLCA, BRCA, HNSCC and PAAD cancers used in the initial set of experiment, each of the datasets had subpopulations that were classified as being similar in expression to a bronchioid, magnoid or squamoid subtype of LUAD (see FIGS. 13-16) and the subpopulations that classified as a bronchioid subtype regardless of cancer type consistently demonstrated lower levels of drug targets and proliferation. This was very similar to what was observed for the LUAD dataset (see FIGS. 13-16 as compared to FIG. 2). Overall, this suggested that one or more tests could be applied to identify patients more appropriately treated by antifolates that include assessing the expression levels of the five (5) pemetrexed drug targets, proliferation or subtyping using the 48 gene LUAD subtyper shown in Table 1 or 4 as provided herein. Each of these tests could be performed alone or in any combination in order to classify cancer subpopulations as being candidates for treatment with antifolates.

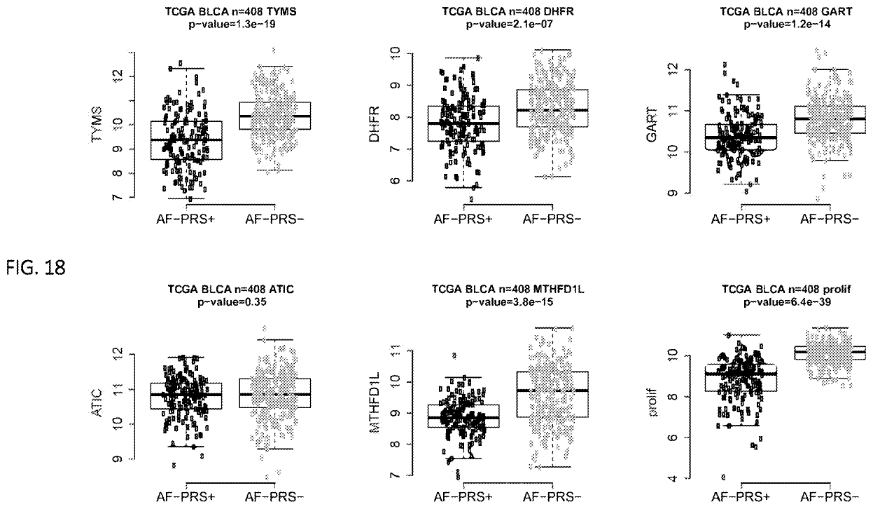

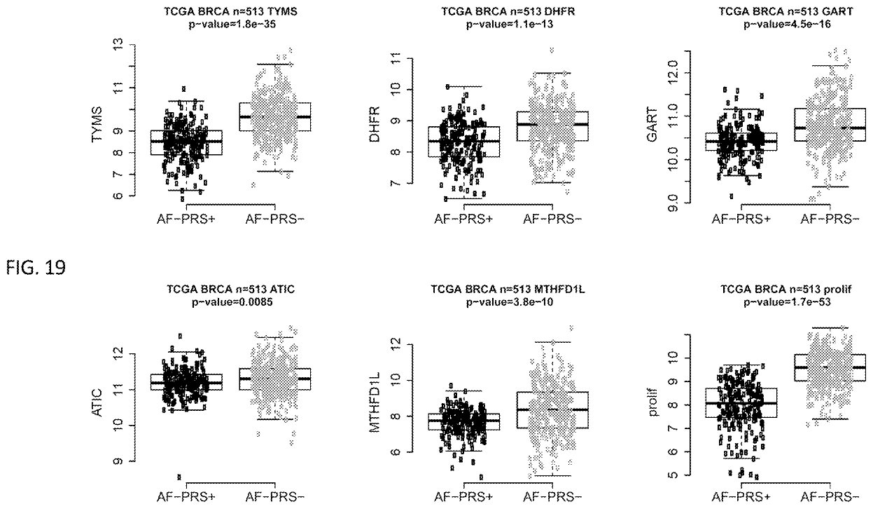

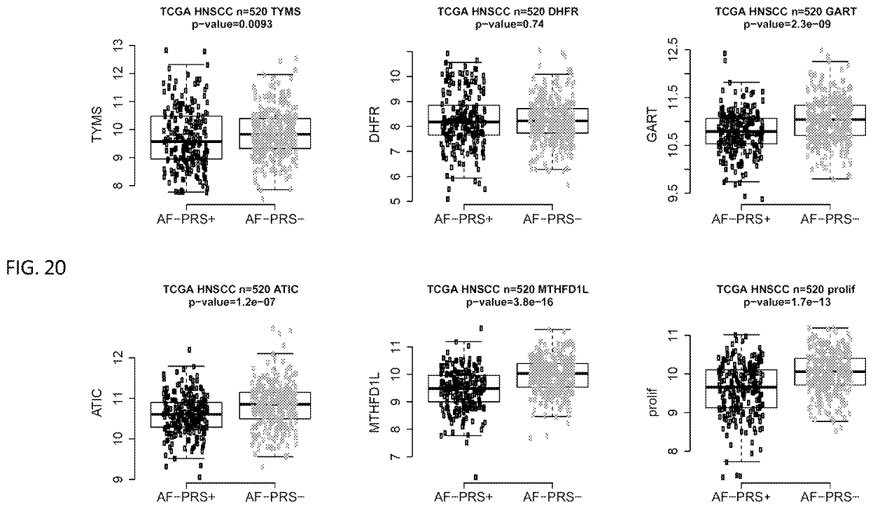

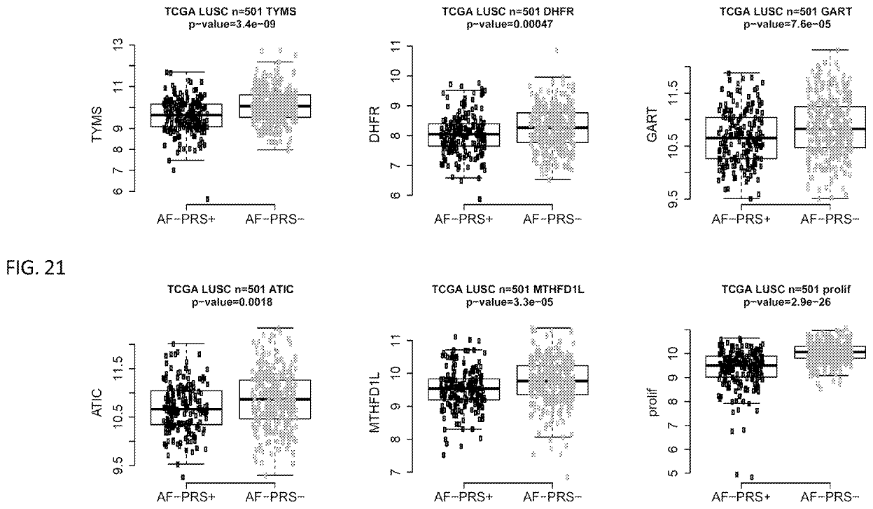

Based on the data, it seems that the results of the subtyping of various cancers using the 48-gene LUAD subtyper of Table 4, (see, for example, FIGS. 13-16) can be regrouped such that the bronchioid subtype for each type of cancer tested (i.e., BLCA, BRCA, HNSCC, and LUSC) can be classified as one group (i.e., anti-folate predictive response signature (AF-PRS)+), while the other subtypes of LUAD (i.e., magnoid and squamoid) can be reclassified as a second group (i.e., AF-PRS −). The results of this re-grouping can be seen in FIG. 18 for BLCA, FIG. 19 for BRCA, FIG. 20 for HNSCC and FIG. 21 for LUSC. Overall, the results obtained by the analysis of LUAD patient gene expression data using the 48-gene LUAD subtyper of WO2017/201165 can be used as an antifolate predictive response signature (AF-PRS) for grouping patients as being AF-PRS (+) and thus likely to be responsive to anti-folate treatment or AF-PRS(−) and thus unlikely to respond to antifolate treatment across numerous cancer types. This AF-PRS can be used alone to assess antifolate predictive response or can be used in conjunction with or as an adjunct to assessing proliferation and/or expression levels of known pemetrexed drug targets.

TABLE 5
Gene Centroids of 60 Classifier Biomarkers for the Bladder Cancer
Subtypes as recreated from Table 1 of PCT/US2019/017799

| | | | II | | | GenBank |
|---|---|---|---|---|---|---|
| | | I | (Luminal | III | IV | Accession |
| Gene Symbol | Gene Name | (Luminal) | Infiltrated) | (Basal) | (Neuronal) | Number* |
| ALDH1L2 | aldehyde | −2.95 | 0.51 | 0.59 | 1.31 | NC_000012.12 |
| | dehydrogenase 1 | | | | | |
| | family member L2 | | | | | |
| ANXA6 | annexin A6 | −2.00 | 0.64 | 0.09 | 1.66 | NC_000005.10 |
| ARSI | arylsulfatase family | −3.67 | 0.42 | 1.72 | 1.23 | NC_000005.10 |
| | member I | | | | | |
| BCAS1 | breast carcinoma | 1.88 | 1.10 | −3.83 | −5.58 | NC_000020.11 |
| | amplified sequence 1 | | | | | |
| BNC1 | basonuclin 1 | −1.48 | −0.57 | 7.97 | 2.24 | NC_000015.10 |
| C10orf99 | chromosome 10 open | 1.69 | 1.15 | −1.26 | −7.80 | NC_000010.11 |
| | reading frame 99 | | | | | |
| C17orf28 | HID1 domain containing | 0.80 | 0.61 | −3.52 | −2.18 | NC_000017.11 |
| CAPN5 | calpain 5 | 1.17 | 1.17 | −2.49 | −1.86 | NC_000011.10 |
| CCDC80 | coiled-coil domain | −3.08 | 1.14 | 0.43 | 1.87 | NC_000003.12 |
| | containing 80 | | | | | |
| COL6A2 | collagen type VI | −2.25 | 1.10 | 0.36 | 2.02 | NC_000021.9 |
| | alpha 2 chain | | | | | |
| CPXM2 | carboxypeptidase X, | −2.14 | 1.78 | −0.13 | 1.70 | NC_000010.11 |
| | M14 family member 2 | | | | | |
| CTHRC1 | collagen triple helix | −3.30 | 0.61 | 0.64 | 1.15 | NC_000008.11 |
| | repeat containing 1 | | | | | |
| DSG3 | desmoglein 3 | −2.16 | −1.59 | 7.87 | −1.24 | NC_000018.10 |
| EMILIN1 | elastin microfibril | −1.96 | 1.86 | −0.20 | 1.84 | NC_000002.12 |
| | interfacer 1 | | | | | |
| EPN3 | epsin 3 | 0.61 | 0.45 | −0.73 | −2.60 | NC_000017.11 |
| EVPL | envoplakin | 0.51 | 0.48 | −0.65 | −2.53 | NC_000017.11 |
| FAP | fibroblast activation | −3.87 | 0.94 | 0.84 | 1.46 | NC_000002.12 |
| | protein alpha | | | | | |
| FBN1 | fibrillin 1 | −2.47 | 1.02 | 0.22 | 1.82 | NC_000015.10 |
| FGF7 | fibroblast growth | −2.23 | 1.98 | 0.12 | 1.44 | NC_000015.10 |
| | factor 7 | | | | | |
| FMO9P | flavin containing | 2.37 | 3.22 | −3.99 | −5.26 | NC_000001.11 |
| | monooxygenase 9 | | | | | |
| | pseudogene | | | | | |
| FNDC1 | fibronectin type III | −4.02 | 2.05 | 0.42 | 2.44 | NC_000006.12 |
| | domain containing 1 | | | | | |
| GABBR2 | gamma-aminobutyric | 0.63 | 5.25 | −2.87 | −0.72 | NC_000009.12 |
| | acid type B receptor | | | | | |
| | subunit 2 | | | | | |
| GFPT2 | glutamine-fructose-6- | −4.37 | 0.83 | 0.74 | 1.95 | NC_000005.10 |
| | phosphate | | | | | |
| | transaminase 2 | | | | | |
| GGT6 | gamma- | 1.18 | 0.33 | −2.23 | −5.61 | NC_000017.11 |
| | glutamyltransferase 6 | | | | | |
| GREM1 | gremlin 1, DAN | −5.82 | 1.63 | 0.66 | 0.50 | NC_000015.10 |
| | family BMP antagonist | | | | | |
| GRHL3 | grainyhead like | 1.02 | 1.46 | −1.39 | −6.23 | NC_000001.11 |
| | transcription factor 3 | | | | | |
| IL20RB | interleukin 20 | −0.89 | −0.97 | 4.28 | −0.59 | NC_000003.12 |
| | receptor subunit beta | | | | | |
| KRT6A | keratin 6A | −2.15 | −2.48 | 7.61 | −0.59 | NC_000012.12 |
| KRT6B | keratin 6B | −1.53 | −2.24 | 7.55 | −0.39 | NC_000012.12 |
| KRT6C | keratin 6C | −1.76 | −2.57 | 7.25 | −1.05 | NC_000012.12 |
| LMOD1 | leiomodin 1 | −1.31 | 2.77 | −0.22 | 0.96 | NC_000001.11 |
| LOC100188947 | HECTD2 antisense | 3.20 | 2.48 | −3.88 | −4.38 | NC_000010.11 |
| | RNA 1 | | | | | |
| MRVI1 | murine retrovirus | −1.19 | 1.65 | −0.15 | 0.54 | NC_000011.10 |
| | integration site 1 | | | | | |
| | homolog | | | | | |
| NRP2 | neuropilin 2 | −2.34 | 0.40 | 0.62 | 1.43 | NC_000002.12 |
| PDLIM3 | PDZ and LIM domain 3 | −2.49 | 1.95 | 0.33 | 1.51 | NC_000004.12 |
| PLA2G4F | phospholipase A2 | 1.13 | 0.24 | −0.66 | −4.12 | NC_000015.10 |
| | group IVF | | | | | |
| PODN | podocan | −1.54 | 1.82 | −0.72 | 1.20 | NC_000001.11 |
| POSTN | periostin | −4.38 | 1.37 | 0.46 | 1.42 | NC_000013.11 |
| PRRX1 | paired related | −3.33 | 0.94 | 0.50 | 2.06 | NC_000001.11 |
| | homeobox 1 | | | | | |
| PVRL4 | nectin cell adhesion | 0.49 | 0.36 | −0.39 | −2.97 | NC_000001.11 |
| | molecule 4 | | | | | |
| RAPGEFL1 | Rap guanine | 1.01 | 0.10 | −0.43 | −3.66 | NC_000017.11 |
| | nucleotide exchange | | | | | |
| | factor like 1 | | | | | |
| RHOU | ras homolog family | 0.59 | 1.22 | −2.78 | −1.70 | NC_000001.11 |
| | member U | | | | | |
| RHOV | ras homolog family | −0.17 | 0.20 | 0.89 | −2.93 | NC_000015.10 |
| | member V | | | | | |
| SCUBE2 | signal peptide, CUB | 1.29 | 3.00 | −3.51 | −1.86 | NC_000011.10 |
| | domain and EGF like | | | | | |
| | domain containing 2 | | | | | |
| SDC1 | syndecan 1 | 0.39 | 0.13 | −0.01 | −2.41 | NC_000002.12 |
| SERPINB13 | serpin family B | −0.64 | −2.10 | 5.68 | −2.94 | NC_000018.10 |
| | member 13 | | | | | |
| SFRP2 | secreted frizzled | −6.65 | 2.50 | 0.68 | 1.68 | NC_000004.12 |
| | related protein 2 | | | | | |
| SFRP4 | secreted frizzled | −5.67 | 3.09 | 0.38 | 2.19 | NC_000007.14 |
| | related protein 4 | | | | | |
| SLC30A2 | solute carrier | 2.17 | 3.26 | −4.60 | −3.18 | NC_000001.11 |
| | family 30 member 2 | | | | | |
| SMOC2 | SPARC related | −1.41 | 2.14 | −0.44 | 1.00 | NC_000006.12 |
| | modular calcium | | | | | |
| | binding 2 | | | | | |
| SNX31 | sorting nexin 31 | 1.40 | 2.05 | −6.48 | −7.24 | NC_000008.11 |
| SPRR2A | small proline rich | −1.49 | −0.32 | 5.64 | −1.95 | NC_000001.11 |
| | protein 2A | | | | | |
| SSC5D | scavenger receptor | −2.38 | 1.82 | 0.09 | 1.78 | NC_000019.10 |
| | cysteine rich family | | | | | |
| | member with 5 domains | | | | | |
| TBX3 | T-box 3 | 1.48 | 0.55 | −3.24 | −2.08 | NC_000012.12 |
| TLE2 | transducin like | 0.96 | 0.69 | −2.92 | −1.06 | NC_000019.10 |
| | enhancer of split 2 | | | | | |
| TOX3 | TOX high mobility | 2.44 | 2.08 | −6.52 | −5.74 | NC_000016.10 |
| | group box family | | | | | |
| | member 3 | | | | | |
| UPK1A | uroplakin 1A | 1.94 | 2.82 | −5.92 | −6.97 | NC_000019.10 |
| UPK2 | uroplakin 2 | 1.85 | 2.45 | −5.94 | −6.04 | NC_000011.10 |
| UPK3A | uroplakin 3A | 1.88 | 3.61 | −5.65 | −4.89 | NC_000022.11 |
| ZNF750 | zinc finger | −0.07 | 0.52 | 0.82 | −3.97 | NC_000017.11 |
| | protein 750 | | | | | |

*Each GenBank Accession Number is a representative or exemplary GenBank Accession Number for the listed gene and is herein incorporated by reference in its entirety for all purposes. Further, each listed representative or exemplary accession number should not be construed to limit the claims to the specific accession number.

TABLE 6
Gene Centroids of 144 Classifier Biomarkers for the Head & Neck Squamous Cell
Carcinoma (HNSCC) Subtypes as recreated from Table 1 PCT/US2018/045522.

| | | | | | | GenBank |
|---|---|---|---|---|---|---|
| | | Atypical | Mesenchymal | Classical | Basal | Accession |
| Gene Symbol | Gene Name | (AT) | (MS) | (CL) | (BL) | Number* |
| ABCC1 | ATP binding cassette | −2.082462349 | −6.056787955 | 14.59888581 | −4.393196801 | NM_004996 |
| | subfamily C member 1 | | | | | |
| ABCC5 | ATP binding cassette | 6.735673492 | −6.863599358 | 13.37272019 | −11.24755644 | NM_005688 |
| | subfamily C member 5 | | | | | |
| ACTN1 | actinin alpha 1 | −15.80447651 | 9.780116364 | −2.540362155 | 8.160936953 | NM_001130004 |
| ACTR1A | ARP1 actin related | −7.43029722 | 5.851180405 | −10.40965677 | 10.4118965 | NM_005736 |
| | protein 1 homolog A | | | | | |
| ADCY10 | adenylate cyclase 10 | −0.716636613 | −1.221986421 | 12.76526279 | −8.904177745 | NM_018417 |
| AKR1C1 | aldo-keto reductase | −1.10933351 | −6.291183618 | 12.40184171 | −3.263102998 | NM_001353 |
| | family 1 member C1 | | | | | |
| APBB2 | amyloid beta precursor | −15.01146625 | 12.44403993 | −3.898903276 | 5.930295847 | NM_004307 |
| | protein binding family | | | | | |
| | B member 2 | | | | | |
| APOL3 | apolipoprotein L3 | 2.286291323 | 5.440122405 | −11.35875857 | 2.052558416 | NM_014349 |
| AQP3 | aquaporin 3 | 3.750713938 | −5.715868054 | −11.04600183 | 11.23366375 | NM_004925 |
| ATP13A4 | ATPase 13A4 | 12.01816284 | −12.34716568 | 1.995296458 | −1.467805824 | NM_032279 |
| ATP6V1D | ATPase H+ | −7.52217377 | −0.516445247 | −7.608420636 | 14.34606418 | NM_015994 |
| | transporting V1 | | | | | |
| | subunit D | | | | | |
| C16orf57 | U6 snRNA biogenesis | −13.73713236 | 5.333139172 | −2.658346322 | 10.56578203 | NM_024598 |
| | phosphodiesterase 1 | | | | | |
| C6orf168 | failed axon connections | 6.916821415 | −3.918221166 | 12.70167928 | −13.73240719 | NM_032511 |
| | homolog | | | | | |
| CAB39 | calcium binding | −5.824036851 | 0.858140491 | −9.792409733 | 13.18131087 | NM_016289 |
| | protein 39 | | | | | |
| CABYR | calcium binding tyrosine | −0.879225603 | −0.139933807 | 13.33042096 | −10.27836496 | NM_012189 |
| | phosphorylation regulated | | | | | |
| CALD1 | caldesmon 1 | −13.57155338 | 14.00571674 | −0.997301758 | 0.533651105 | NM_033138 |
| CASP4 | caspase 4 | −6.233121399 | 1.302626733 | −11.00541654 | 14.1766376 | NM_001225 |
| CAV1 | caveolin 1 | −13.83491768 | 7.418519883 | −3.94809826 | 9.71830341 | NM_001753 |
| CD276 | CD276 molecule | −13.57242716 | 10.95369333 | 1.01404026 | 1.810966974 | NM_001024736 |
| CD74 | CD74 molecule | 5.84959342 | 7.771329118 | −10.50803194 | −4.449565802 | NM_001025159 |
| CDSN | corneodesmosin | −10.6365512 | −1.493069449 | −4.906096366 | 16.077773 | NM_001264 |
| CEACAM5 | carcinoembryonic | 11.35567594 | −11.26562383 | 0.422805332 | −0.540569935 | NM_004363 |
| | antigen related cell | | | | | |
| | adhesion molecule 5 | | | | | |
| CHPT1 | choline | 9.816276839 | 2.262831082 | 4.328822728 | −15.5331284 | NM_020244 |
| | phosphotransferase 1 | | | | | |
| CHST7 | carbohydrate | 1.127395223 | −1.274663652 | 13.63700234 | −11.40544572 | NM_019886 |
| | sulfotransferase 7 | | | | | |
| CIITA | class II major | 7.849953146 | 5.449974972 | −11.0739908 | −3.674106625 | NM_001286402 |
| | histocompatibility | | | | | |
| | complex transactivator | | | | | |
| CLCN2 | chloride voltage-gated | 1.869955841 | −3.034672968 | 12.48578206 | −9.444551535 | NM_004366 |
| | channel 2 | | | | | |
| CMTM3 | CKLF like MARVEL | −6.94786756 | 14.99532869 | −1.596971364 | −6.444079867 | NM_144601 |
| | transmembrane domain | | | | | |
| | containing 3 | | | | | |
| COCH | cochlin | 7.245471303 | −2.225717996 | 11.06208646 | −14.32016052 | NM_001135058 |
| COL6A1 | collagen type VI | −10.61945956 | 15.09999138 | 0.023929886 | −4.304047285 | NM_001848 |
| | alpha 1 chain | | | | | |
| COL6A2 | collagen type VI | −9.816813759 | 15.40019653 | −0.059414969 | −5.31650074 | NM_001849 |
| | alpha 2 chain | | | | | |
| CREB3L4 | cAMP responsive | 12.35426247 | −1.036014582 | 4.170449139 | −14.67779961 | NM_130898 |
| | element binding | | | | | |
| | protein 3 like 4 | | | | | |
| CSNK1A1 | casein kinase 1 alpha 1 | −4.157095083 | −4.379943874 | −6.408502727 | 13.78896776 | NM_001025105 |
| CSTA | cystatin A | 7.6349338 | −13.92125741 | −4.634101181 | 9.992427797 | NM_005213 |
| CSTB | cystatin B | 5.441142897 | −10.92489787 | −5.097693335 | 9.619831396 | NM_000100 |
| CYP26A1 | cytochrome P450 | 5.568513158 | −4.690925538 | 14.07156336 | −12.8104492 | NM_000783 |
| | family 26 subfamily A | | | | | |
| | member 1 | | | | | |
| CYP4B1 | cytochrome P450 | 14.45700653 | −7.05416422 | −4.703606141 | −3.36503105 | NM_001099772 |
| | family 4 subfamily B | | | | | |
| | member 1 | | | | | |
| DHRS1 | dehydrogenase/ | −0.193215811 | −6.493438067 | −9.311117789 | 14.40626466 | NM_001136050 |
| | reductase 1 | | | | | |
| DSG1 | desmoglein 1 | −7.614095504 | −3.451410234 | −3.737838114 | 14.0254529 | NM_001942 |
| ELF3 | E74 like ETS | 13.68731232 | −13.60589315 | 4.343709261 | −3.869532938 | NM_004433 |
| | transcription factor 3 | | | | | |
| EPCAM | epithelial cell adhesion | 4.265932752 | −6.736175943 | 13.73306161 | −9.245971416 | NM_002354 |
| | molecule | | | | | |
| EPGN | epithelial mitogen | −5.618779265 | −3.300485871 | −6.577764094 | 14.31742891 | NM_001270989 |
| EYA2 | EYA transcriptional | 15.04545577 | −2.190930112 | 1.591800862 | −14.01747204 | NM_005244 |
| | coactivator and | | | | | |
| | phosphatase 2 | | | | | |
| F2RL1 | F2R like trypsin | −13.80547895 | 2.765020504 | 1.370242141 | 9.730255475 | NM_005242 |
| | receptor 1 | | | | | |
| FAM171A1 | family with sequence | 9.195667146 | 2.45871035 | 3.837122752 | −14.69736658 | NM_001010924 |
| | similarity 171 member A1 | | | | | |
| FAM3B | family with sequence | 23.49686554 | −10.23769328 | 1.726344969 | −14.59685293 | NM_058186 |
| | similarity 3 member B | | | | | |
| FAM40A | striatin interacting | 0.072030977 | −1.193725622 | −11.40053476 | 10.74128539 | NM_033088 |
| | protein 1 | | | | | |
| FBLIM1 | filamin binding LIM | −13.8444937 | 3.484519683 | −5.129731741 | 14.56682766 | NM_017556 |
| | protein 1 | | | | | |
| FGD2 | FYVE, RhoGEF and | 6.075397444 | 5.847611036 | −10.21140803 | −3.045464114 | NM_173558 |
| | PH domain containing 2 | | | | | |
| FKBP9 | FK506 binding | −13.43106274 | 7.315547198 | 4.438931756 | 2.324123909 | NM_007270 |
| | protein 9 | | | | | |
| FN1 | fibronectin 1 | −10.72721251 | 14.68092168 | 1.901951252 | −5.378217455 | NM_212482 |
| FOXA1 | forkhead box A1 | 17.64133976 | −11.07111965 | 6.438446491 | −12.00758435 | NM_004496 |
| FSTL3 | follistatin like 3 | −15.00524247 | 9.744960025 | −2.21225958 | 7.130950667 | NM_005860 |
| FUT6 | fucosyltransferase 6 | 16.26859049 | −12.17798039 | −2.993362909 | −1.594985757 | NM_000150 |
| GALNT6 | polypeptide N-acetyl | −13.07407145 | 2.051509658 | −3.432309832 | 13.77063593 | NM_007210 |
| | galactosaminy1 | | | | | |
| | transferase 6 | | | | | |
| GCNT2 | glucosaminyl (N- | 11.00945937 | −5.005886796 | 12.29522358 | −16.35521859 | NM_145649 |
| | acetyl) transferase | | | | | |
| | 2, I-branching enzyme | | | | | |
| | (I blood group) | | | | | |
| GLS2 | glutaminase 2 | 15.18663893 | −8.147146843 | 8.499138056 | −14.18880539 | NM_013267 |
| GPR110 | G-protein coupled | 12.7116071 | −11.65119722 | −3.963069483 | 2.212488501 | AY140952 |
| | receptor GPR110 | | | | | |
| GPRC5B | G protein-coupled | 5.833385675 | 5.048036921 | 4.465010876 | −14.44626312 | NM_016235 |
| | receptor class C | | | | | |
| | group 5 member B | | | | | |
| GPX8 | glutathione peroxidase | −10.3612256 | 14.94672566 | 0.015638923 | −4.401628144 | NM_001008397 |
| | 8 (putative) | | | | | |
| GRHL3 | grainy head like | 7.100258671 | −14.0190496 | −0.688942122 | 7.275695546 | NM_021180 |
| | transcription factor 3 | | | | | |
| GSDMA | gasdermin A | −9.15951728 | −1.729489137 | −5.968036589 | 15.75333543 | NM_178171 |
| HEY1 | hes related family | 0.159448658 | 3.534415289 | 12.27328877 | −13.99193179 | NM_012258 |
| | bHLH transcription | | | | | |
| | factor with YRPW | | | | | |
| | motif 1 | | | | | |
| HLA-DRA | HLA-DRA | 5.635788952 | 7.540528354 | −10.23102672 | −4.248292697 | KY497357 |
| HLF | HLF, PAR bZIP | 16.77558527 | −10.04027801 | 4.574705233 | −10.58434418 | NM_002126 |
| | transcription factor | | | | | |
| IKZF2 | IKAROS family zinc | 14.98317462 | −6.074474772 | −3.858000118 | −5.554553773 | NM_016260 |
| | finger 2 | | | | | |
| IL4R | interleukin 4 receptor | 0.217776544 | 0.457253449 | −13.81453534 | 11.02939058 | NM_000418 |
| INHBA | inhibin beta A subunit | −14.79489264 | 8.427676625 | −0.696922701 | 6.927158767 | NM_002192 |
| KIAA1609 | KIAA1609 | −9.8340117 | 2.292555067 | −8.206314175 | 14.38614729 | AB046829 |
| KLF5 | Kruppel like factor 5 | 4.562842302 | −13.00491807 | 3.89026837 | 4.908510892 | NM_001730 |
| LEPRE1 | prolyl 3-hydroxylase 1 | −11.83578491 | 16.04587546 | 0.555964732 | −4.480170241 | NM_022356 |
| LMO4 | LIM domain only 4 | 14.78188317 | −4.286105968 | 2.544496006 | −12.51954481 | NM_006769 |
| LOC643008 | small integral | 12.65412229 | −11.504798 | −3.985040113 | 2.144789085 | XM_017024943 |
| | membrane protein 5 | | | | | |
| LRIG1 | leucine rich repeats | 6.317083957 | 2.548224024 | 6.751703803 | −14.41779751 | NM_015541 |
| | and immunoglobulin | | | | | |
| | like domains 1 | | | | | |
| LTBP3 | latent transforming | 3.235488698 | 7.629013681 | 6.180250436 | −15.85948808 | NM_001130144 |
| | growth factor beta | | | | | |
| | binding protein 3 | | | | | |
| MAL2 | mal, T-cell | 6.413419457 | −11.71334434 | −0.431651323 | 5.483862841 | NM_052886 |
| | differentiation protein 2 | | | | | |
| MAP7D1 | MAP7 domain | −8.910849678 | 3.124678656 | −10.08235276 | 14.25296762 | NM_018067 |
| | containing 1 | | | | | |
| MEIS1 | Meis homeobox 1 | 17.32037654 | −5.648950707 | −2.757291231 | −9.201660614 | NM_002398 |
| MMP1 | matrix metallopeptidase 1 | −13.26866191 | 6.822304749 | −2.002155679 | 8.096135704 | NM_002421 |
| MOBKL2B | MOB kinase activator 3B | −2.913703778 | 4.221634948 | −11.2899732 | 8.301609003 | NM_024761 |
| MPPED1 | metallophosphoesterase | 0.75117262 | −3.628778766 | 12.92063032 | −8.131564761 | NM_001044370 |
| | domain containing 1 | | | | | |
| MRAP2 | melanocortin 2 receptor | 8.821566644 | −4.975238292 | 12.75983052 | −14.62483234 | NM_138409 |
| | accessory protein 2 | | | | | |
| MUC20 | mucin 20, cell surface | 15.44317871 | −10.50413061 | 2.271620266 | −6.871349665 | NM_001282506 |
| | associated | | | | | |
| MUC4 | mucin 4, cell surface | 15.96069019 | −8.652630447 | 2.582388666 | −9.450568232 | NM_018406 |
| | associated | | | | | |
| MYB | MYB proto-oncogene, | 15.0257076 | −5.385618207 | 5.6522876 | −14.3163554 | NM_001130173 |
| | transcription factor | | | | | |
| NNMT | nicotinamide N- | −10.09900408 | 15.95020532 | −3.274573382 | −2.854813073 | NM_006169 |
| | methyltransferase | | | | | |
| NSUN7 | NOP2/Sun RNA | 16.08489314 | −4.722807369 | 6.335643561 | −16.58396549 | NM_024677 |
| | methyltransferase | | | | | |
| | family member 7 | | | | | |
| NTRK2 | neurotrophic receptor | 10.23093315 | −6.376330063 | 12.76453966 | −14.64867156 | NM_006180 |
| | tyrosine kinase 2 | | | | | |
| OLFML2B | olfactomedin like 2B | −6.80300319 | 15.66852662 | −0.83099608 | −7.891812186 | NM_001297713 |
| OLFML3 | olfactomedin like 3 | −6.546734956 | 16.36834609 | −3.370305787 | −6.678215459 | NM_020190 |
| P4HTM | prolyl 4-hydroxylase, | 11.8274774 | 2.672495061 | 5.253671456 | −18.69507373 | NM_177939 |
| | transmembrane | | | | | |
| PAQR5 | progestin and adipoQ | −10.21974925 | −0.676386205 | −2.846883938 | 13.12800887 | NM_001104554 |
| | receptor family member 5 | | | | | |
| PATZ1 | POZ/BTB and AT | 8.493850484 | 2.966225133 | 7.456620531 | −17.56473037 | NM_014323 |
| | hook containing zinc | | | | | |
| | finger 1 | | | | | |
| PBX1 | PBX homeobox 1 | 13.2718934 | −1.583812326 | 4.352097293 | −15.20010123 | NM_002585 |
| PCOLCE | procollagen C- | −8.027597076 | 16.0810449 | 0.683124494 | −8.370334 | NM_002593 |
| | endopeptidase enhancer | | | | | |
| PHLDB1 | pleckstrin homology | −11.25408443 | 15.15862534 | −1.738679371 | −2.245099413 | NM_015157 |
| | like domain family B | | | | | |
| | member 1 | | | | | |
| PIR | pirin | 7.490180987 | −9.475330645 | 13.12744434 | −9.233843088 | NM_003662 |
| PKP3 | plakophilin 3 | −5.164891557 | −4.425600449 | −5.236735217 | 13.83389626 | NM_007183 |
| PLAC8 | placenta specific 8 | 16.72602757 | −4.212412233 | 1.082753487 | −13.26806322 | NM_001130716 |
| PLD2 | phospholipase D2 | −4.518197205 | −5.092960256 | −7.348969116 | 15.63604839 | NM_002663 |
| PMP22 | peripheral myelin | −7.194357544 | 14.68864942 | 1.396604647 | −8.43537032 | NM_000304 |
| | protein 22 | | | | | |
| PPARD | peroxisome proliferator | −7.071754506 | −3.884764304 | −4.912078226 | 14.90821141 | NM_006238 |
| | activated receptor delta | | | | | |
| PPL | periplakin | 6.261655734 | −12.16995239 | −1.756388798 | 7.199844946 | NM_002705 |
| PRKX | protein kinase, X- | 8.851366302 | −8.998241561 | 13.1773563 | −11.08142743 | NM_005044 |
| | linked | | | | | |
| PRSS27 | protease, serine 27 | 9.329134265 | −10.80925456 | −5.741713479 | 6.225164659 | NM_031948 |
| PTH1R | parathyroid hormone 1 | −6.722598413 | 14.82555669 | −0.472460255 | −7.45169274 | NM_000316 |
| | receptor | | | | | |
| PTRF | caveolae associated | −13.14332726 | 9.468944832 | −4.638965523 | 7.621207859 | NM_012232 |
| | protein 1 | | | | | |
| RAB25 | RAB25, member RAS | 5.219572333 | −11.52664115 | −0.156829226 | 6.244155734 | NM_020387 |
| | oncogene family | | | | | |
| RAB6B | RAB6B, member RAS | −1.603161781 | 0.110021996 | 15.94132967 | −12.01912932 | NM_016577 |
| | oncogene family | | | | | |
| RCN3 | reticulocalbin 3 | −8.161767875 | 14.46620853 | 1.654069744 | −7.483978219 | NM_020650 |
| RFTN1 | raftlin, lipid raft | −4.573623438 | 9.589915176 | −10.67106662 | 4.172768882 | NM_015150 |
| | linker 1 | | | | | |
| RGS20 | regulator of G protein | −12.52290526 | 2.810238619 | −4.159943794 | 13.1034325 | NM_170587 |
| | signaling 20 | | | | | |
| RIMKLA | ribosomal modification | 7.013215205 | −3.910313002 | 13.32678642 | −14.36397185 | NM_173642 |
| | protein rimK like family | | | | | |
| | member A | | | | | |
| SCARA3 | scavenger receptor | 8.486922621 | 2.228910754 | 4.488976365 | −14.32712171 | NM_016240 |
| | class A member 3 | | | | | |
| SCNN1A | sodium channel | 9.314590424 | −10.85586757 | 4.480301787 | −2.364983519 | NM_001038 |
| | epithelial 1 alpha | | | | | |
| | subunit | | | | | |
| SERPINE1 | serpin family E | −14.50591442 | 8.635050854 | 1.480793338 | 4.597552116 | NM_000602 |
| | member 1 | | | | | |
| SERPINH1 | serpin family H | −14.74205489 | 14.20433013 | 0.878953362 | −0.095802953 | NM_001207014 |
| | member 1 | | | | | |
| SETMAR | SET domain and | 12.83440944 | 0.233932813 | 2.138469148 | −14.67025168 | NM_006515 |
| | mariner transposase | | | | | |
| | fusion gene | | | | | |
| SFXN3 | sideroflexin 3 | −13.93133999 | 11.56847653 | −6.74034327 | 8.126085326 | NM_030971 |
| SGEF | SH3-containing | 15.50488845 | −8.827171693 | 14.7491026 | −19.12717961 | AY552599 |
| | guanine nucleotide | | | | | |
| | exchange factor | | | | | |
| SH2D4A | SH2 domain containing 4A | 14.91939392 | −10.45712779 | −1.285782521 | −3.391377233 | NM_022071 |
| SH2D5 | SH2 domain containing 5 | −13.29966495 | 1.411309304 | −2.686017395 | 13.9859309 | NM_001103161 |
| SLAMF7 | SLAM family member 7 | 4.242417997 | 2.960164251 | −10.72277915 | 2.009246005 | NM_021181 |
| SLC16A14 | solute carrier family 16 | 8.453385598 | −5.352426361 | 12.76874135 | −13.90188986 | NM_152527 |
| | member 14 | | | | | |
| SLC31A2 | solute carrier family 31 | −8.009411845 | 6.173856422 | −11.1271606 | 11.27415259 | NM_001860 |
| | member 2 | | | | | |
| SLC9A3R1 | SLC9A3 regulator 1 | 9.971190685 | −12.54044249 | 4.659221949 | −1.518674303 | NM_004252 |
| SNAI2 | snail family | −14.55807791 | 7.947052877 | 0.055367787 | 6.526518445 | NM_003068 |
| | transcriptional | | | | | |
| | repressor 2 | | | | | |
| SPARC | secreted protein acidic | −9.442299973 | 14.5249952 | 0.460899979 | −5.27130454 | NM_003118 |
| | and cysteine rich | | | | | |
| SPINK5 | serine peptidase | 8.887143269 | −10.88801305 | −5.380297697 | 6.431225145 | NM_001127698 |
| | inhibitor, Kazal type 5 | | | | | |
| TAGLN | transgelin | −8.774403137 | 14.78574024 | 1.128839097 | −6.748363792 | NM_001001522 |
| TBXA2R | thromboxane A2 receptor | −7.041663263 | 14.43062303 | −1.279759745 | −6.069091489 | NM_001060 |
| TGFB3 | transforming growth | −5.513659516 | 14.73938538 | −3.22553509 | −6.227831211 | NM_003239 |
| | factor beta 3 | | | | | |
| TGFBI | transforming growth | −15.54040671 | 11.61576363 | −3.305840607 | 6.757370024 | NM_000358 |
| | factor beta induced | | | | | |
| TJP3 | tight junction protein 3 | 17.32770506 | −9.778563744 | −1.776083891 | −6.00908976 | NM_001267560 |
| TMEM51 | transmembrane protein 51 | 2.637524229 | 3.608309483 | −13.856265 | 5.607931398 | NM_001136216 |
| TMPRSS11A | transmembrane | 14.96390452 | −13.07259679 | 1.124526863 | −2.922374221 | NM_182606 |
| | protease, serine 11A | | | | | |
| TMPRSS11B | transmembrane | 16.17986512 | −11.62554287 | −1.63009884 | −3.200386118 | NM_182502 |
| | protease, serine 11B | | | | | |
| TMPRSS2 | transmembrane | 19.36438158 | −10.34381575 | 2.078270847 | −10.72366129 | NM_001135099 |
| | protease, serine 2 | | | | | |
| TTC9 | tetratricopeptide repeat | 11.89329997 | −12.38268834 | −1.756842725 | 1.864848764 | NM_015351 |
| | domain 9 | | | | | |
| TXNRD1 | thioredoxin reductase 1 | −0.999104468 | −3.250337096 | 14.50623299 | −8.119912193 | NM_182729 |
| UBA7 | ubiquitin like modifier | 5.929616343 | 4.581359308 | −11.05993504 | −0.948212309 | NM_003335 |
| | activating enzyme 7 | | | | | |
| VAMP3 | vesicle associated | −3.668365448 | 1.73966571 | −10.63156161 | 10.90939129 | NM_004781 |
| | membrane protein 3 | | | | | |
| VAV3 | vav guanine nucleotide | 4.420409601 | −0.330479172 | −10.38204488 | 4.757063432 | NM_006113 |
| | exchange factor 3 | | | | | |
| VEGFC | vascular endothelial | −13.74904145 | 10.29391086 | −8.204386968 | 10.42939104 | NM_005429 |
| | growth factor C | | | | | |
| ZDHHC2 | zinc finger DHHC-type | 9.037395105 | 0.904115321 | 11.9082815 | −19.85433707 | NM_016353 |
| | containing 2 | | | | | |

*Each GenBank Accession Number is a representative or exemplary GenBank Accession Number for the listed gene and is herein incorporated by reference in its entirety for all purposes. Further, each listed representative or exemplary accession number should not be construed to limit the claims to the specific accession number.

TABLE 7
PAAD subtyper or signature as
recreated from Table 9 of US2017/0233927
Fitted Estimates for the Final Model

| | | | Estimated Increase |
|---|---|---|---|
| | | | in Odds of [symbol] Class |
| Gene A | Gene B | Coefficient | Membership when A > B |
| CD100 | GPR [symbol] | 0.87 | 2.38 |
| SLC2A1 | AG [symbol] | 1.22 | 3.39 |
| KRT16 | SLC44A4 | 0.52 | 1.68 |
| CTS [symbol] | TMEM45B | 1.43 | 4.17 |
| KRT [symbol] | BCAS1 | 0.70 | 2.01 |
| B3GNT5 | VSIG2 | 0.41 | 1.51 |
| MET | TFF3 | 0.72 | 2.06 |
| CHST6 | PLA22G10 | 0.80 | 2.24 |
| SERPINEB5 | BFGD | 0.76 | 2.13 |
| DCBLD2 | PLS1 | 1.40 | 4.07 |
| IL20 [symbol] | FAM3D | 1.33 | 3.70 |
| PPP [symbol] | SYTL2 | 1.58 | 4.85 |
| NAB1 | PLEKHA6 | 0.41 | 1.50 |
| MSLN | CAPN [symbol] | 1.58 | 4.83 |
| | (Intercept) | −7.16 | |

[symbol]

indicates data missing or illegible when filed

### Example 6—Examination on the Use of AF-PRS as a Potential Immunotherapy Predictive Response Indicator

Previous studies have shown that the bronchioid, magnoid and squamoid subtypes differ in their genomic alterations and potential treatment response to immunotherapy (see Wilkerson et al. PLOS One 2012. PMID: 22590557 and TCGA. Nature. 2014 Jul. 31; 511(7511):543-50. PMID:25079552). In 12 months from now we will have additional real-world data using the AF-PRS in NS-NSCLC and potentially bladder which will allow us to demonstrate additional utility with pemetrexed treatment vs. pembrolizumab that we can include when we convert to a full patent.

Pemetrexed plus platinum-based antineoplastic drugs (informally called platins or platinum) has been recently approved for the cotreatment of patients with a PD-L1 inhibitor (pembrolizumab). Based on the intrinsic differences between the LUAD subtypes, this first line treatment may not be appropriately treating the bronchioid subtype adenocarcinoma patients. The results from Example 4 (see FIGS. 6 and 7) certainly suggests that the bronchioid subtype may be better treated by pemetrexed plus platinum, while squamoid subtype may be better treated with PD-L1 inhibition. This suggests that the activity of recently approved combined treatment (i.e., pemetrexed plus platinum plus PD-L1 inhibitor) may be additive and not synergistic with two distinct molecular subtypes being treated with the triplet therapy and showing clinical improvement for non-small cells/non-squamous lung cancer patients as a whole; however, but the correct drug treatment may not be getting to the right patient in all cases.

In order to examine this possibility, the AF-PRS signature provided herein will be used as described herein to classify patient subpopulations for lung adenocarcinoma as well as bladder cancer as being either AF-PRS (+) or AF-PRS (−). Subsequently, the survival (e.g., overall survival or progression-free survival) within specific treatment regimens containing pemetrexed (e.g., pemetrexed monotherapy, pemetrexed plus platinum or triplet therapy (pemetrexed plus platinum plus PD-L1 inhibitor)) will be compared between AF-PRS (+) versus AF-PRS (−) populations. Similarly, the survival (e.g., overall survival or progression-free survival) in response to specific treatment regimens containing pemetrexed (e.g., pemetrexed monotherapy, pemetrexed plus platinum or triplet therapy (pemetrexed plus platinum plus PD-L1 inhibitor)) will be examined in the AF-PRS (+) and/or (−) groups. These examinations will be conducted as non-interventional retrospective or prospective studies with tumor samples and clinical data collected for patients undergoing treatment with pemetrexed-containing therapy or other standard of care therapy. Similarly, these examinations will be conducted using prospective interventional clinical studies where response to pemetrexed-containing therapy will be compared to either a parallel standard of care arm or previously collected retrospective tumor and clinical response data.

### Example 7—Evaluation of an Antifolate Response Signature (AF-PRS) and its Association with Survival in Non-Small Cell Lung Cancer (NSCLC) Patients Treated with Pemetrexed-Containing Platinum Doublet Chemotherapy (PMX-PDC)—the Piedmont Study

### Introduction

It is estimated that there were 235,760 new cases of lung cancer and 131,800 deaths in 2021 (www.cancer.gov). Lung cancer is the 3^{rd} most common type of cancer but results in the greatest number of deaths of all cancer types. A vast majority (84%; 198,038) of lung cancer diagnoses are non-small cell lung cancer (NSCLC) (www.cancer.gov). Most patients (53.9%) are metastatic (Stage IV) at diagnosis with the remainder Stage I-III (Wang et al, 2018). For newly diagnosed, relapsed or recurrent Stage IV NSCLC patients, treatments include surgery, radiation and/or systemic therapies (e.g., cytotoxic chemotherapy, targeted therapy, immune therapy). For patients with Stage I-III NSCLC, surgery is the primary treatment with the addition of radiation and/or systemic therapies.

Platinum doublet chemotherapy (PDC; cisplatin or carboplatin combined with a second chemotherapeutic agent) has been a mainstay systemic treatment of NSCLC since the original approval of vinorelbine+cisplatin in 1989, and subsequent approval of other PDC combinations including gemcitabine and taxanes. These PDC options were used across the broader NSCLC patient population independent of histology and provided for similar modest but clinically meaningful improvement in survival over non-systemic standards of care, including surgery and radiation (Reviewed in Baxevanos and Mountzios, 2018). The particular PDC used was typically based upon the tolerability profile and not based upon histology or molecular characteristics.

Pemetrexed belongs to a class of chemotherapy agents that target the folate pathway by interfering with the production of purine and pyrimidine nucleotides—and hence DNA and RNA synthesis—by inhibiting shared enzymes, thymidylate synthase (TYMS) and dihydrofolate reductase (DHFR) as well as the purine biosynthetic pathway-specific enzymes phosphoribosylglycinamide formyltransferase (GART) and 5-aminoimidazole-4-carboxamide ribonucleotide formyltransferase/IMP cyclohydrolase (ATIC), thereby disrupting folate-dependent metabolism essential to proliferating cancer cells. The initial approval of pemetrexed-containing PDC (PMX-PDC) in 2008 was the first PDC regimen to be approved where patients were selected by histology (patients with nonsquamous (NS)-NSCLC). This approval was based upon a non-inferiority study of pemetrexed+cisplatin versus gemcitabine+cisplatin in patients with Stage IIIB or IV NSCLC (Scagliotti et al, 2008). While survival was similar between both treatment groups, patients with nonsquamous histology (large cell or adenocarcinoma) had superior survival with pemetrexed+cisplatin, but those with squamous histology had inferior survival. PMX-PDC garnered wide use in NS-NSCLC patients, but the approval of single agent pembrolizumab in PD-L1 positive patients or in combination with PMX-PDC in metastatic patients regardless of PD-L1 status has resulted in decreased PMX-PDC use as a stand-alone regimen in Stage IV disease. However, it is still used frequently in earlier stage patients who are indicated for systemic chemotherapy.

Prior attempts at developing novel biomarkers that could be used to predict PMX-PDC response include IHC expression of target proteins such as thymidylate synthase or RNA expression analysis of its gene (TYMS; Sun J M et al., J Thorac Oncol 2011; Chen C Y, et al. Lung Cancer 2011; Sigmond J., et al., Biochem Pharmacol 2003; Takezawa K et al., Br J Cancer, 2011), with a demonstration that protein and/or gene expression is inversely related with pemetrexed activity (REF). Early work by Hayes and colleagues (2006, 2012) evaluated the use of RNA gene expression analysis to identify lung adenocarcinoma (LUAD) molecular subtypes (i.e., bronchioid, magnoid and squamoid) that could be useful in predicting treatment response to various NSCLC treatment options, but this work was not tied directly with PMX-PDC response per se. With the blinded Phase 2 study of TS molecular and protein expression relationship with PMX-PDC response (Nicolson et al, 2013) and subsequent molecular subtype analysis by Fennell et al (2014), the LUAD subtypes developed by Hayes and colleagues (2006, 2012) were utilized for the first time to evaluate pemetrexed response in NS-NSCLC patients. The study in this Example (i.e., the Piedmont study) builds upon these foundational RNA subtyping findings and examines the novel 48 gene antifolate response signature (AF-PRS) provided herein.

### Objective

As part of a larger retrospective study of NS-NSCLC patients treated with standard of care systemic therapies, this Example focused on patients treated PMX-PDC in the Stage I-IV setting. A primary objective was to evaluate a novel RNA-based 48 gene antifolate response signature (AF-PRS) and test the hypothesis that patients who are AF-PRS positive (+) will demonstrate preferential response to PMX-PDC compared to those who are AF-PRS negative (−). The clinical findings were put in context of key genes associated with pemetrexed activity and metabolism to better explain potential preferential responsiveness in AF-PRS(+) patients. The clinical importance of this study is the potential demonstration of initial utility of the AF-PRS, which may be further developed as a diagnostic test to aid in the selection of patients who are indicated for systemic chemotherapy that are most likely to respond to PMX-PDC.

### Materials and Methods

### Patient Samples

As part of a larger retrospective analysis of 240 NS-NSCLC patients treated with standard of case (SOC) therapies, 114 patients were identified as having archived pre-treatment formalin-fixed paraffin embedded (FFPE) tumor tissue samples from a primary or metastatic site with sufficient material from which to extract RNA and DNA and having received treatment with PMX-PDC for locally advanced or metastatic disease. All patients were treated within the Levine Cancer Institute/Atrium Health hospital system (Charlotte, NC) between 2012 and 2020.

### Clinical Annotation

Demographic and clinical variables were collected from medical records and entered into a dedicated auditable database (REDCap; www.project-redcap.org) designed around a pre-defined data dictionary. Data entry and subsequent QC were performed by separate individuals. Baseline clinical variables included information recorded at the time of initiation of PMX-PDC, which was administered as standard of care alone or in combination with other interventions such as surgery or radiation. Overall survival (OS) was defined as the interval from PMX-PDC initiation to patient death. The Social Security Death Index was consulted whenever possible if death date was not available. Progression free survival (PFS) from PDC-PMX was defined as the interval between initiation of initial PMX-PDC treatment and disease progression, or the date of death in the absence of noted disease progression. In cases where a patient was still alive or the date of death was unknown, date of last contact was used in place to estimate the censored OS/PFS. Clinical benefit was defined as complete response (CR), partial response (PR), or stable disease (SD). A total of 90 of 95 patients with suitable RNAseq data (reviewed below) were evaluable for clinical response.

### IRB Approval

Patient samples and corresponding clinical data were collected under an IRB-approved protocol (Levine Cancer Institute) that allowed for the waiver of informed consent for combined analysis of molecular data and relevant clinical and demographic data, provided that necessary protected health information (PHI) was removed, and dates were shifted prior to data transfer and subsequent analysis.

### RNA Sequencing

H&E-stained FFPE sections underwent microscopic QC review by an anatomical pathologist to confirm histology diagnosis, evaluate percent tumor nuclei (≥5% required), percent necrosis and cellularity prior to macrodissection and dual DNA/RNA extraction using the truXTRAC FFPE total nucleic acid kit (Covaris). RNA quantification was performed by Qubit measurement using ribogreen staining. RNA was qualitatively assessed for integrity by Agilent TapeStation gel electrophoresis. RNA samples approved for analysis (optimal requirements included 10 ng by ribogreen quantification and a TapeStation DV200 value ≥20%) underwent library preparation using AmpliSeq for Illumina Transcriptome Human Gene Expression Panel kit. A no template control (NTC) and positive control sample (NA12878 FFPE RNA) were included in each run. Libraries were individually captured, reviewed for appropriate size using a Bioanalyzer or TapeStation trace, and quantified (KAPA library quantification) prior to equal molar pooling. Sequencing was performed on an Illumina NovaSeq6000 sequencer using an S2 flow cell to generate ˜50M, 2×50 bp paired-end reads. RNAseq data were qualified and analyzed against other datasets within GeneCentric's archive. The primary acceptance criteria for RNAseq quality were a median transcriptome-wide correlation of >0.8 and >25% of reads mapped to mRNA bases.

### RNA Expression Analyses

Expression values for the samples were derived from raw RNAseq fastq files. Reads were aligned with STAR-aligner (GrCH38 ver. 22) to human assembly using the STAR/Salmon pipeline (Dobin A et al., Bioinformatics. 2013). Expression was quantified using the RSEM package (Li B et al., BMC Bioinformatics. 2011) and the GrCH38 human transcriptome reference. Genes were filtered for a minimum expression count (at least 10 reads in at least 5 samples), and for a protein coding annotation by Ensemble (final set of genes=16,901). Differential expression was assessed using the DESeq2 package (Love M I et al., Genome Biol. 2014) on this filtered set of genes. For all other analyses, all expression values were log 2(1+x) transformed, median centered and upper quartile scaled.

### Gene Signatures

### 48-Gene NS-NSCLC Nearest Centroid Classifier—

The AF-PRS is the same as the 48-gene LUAD nearest centroid classifier (see WO 2017/201165, which is herein incorporated by reference), except the bronchioid subtype is called AF-PRS (+) and the remaining two subtypes (magnoid and squamoid) are combined as AF-PRS (−). While the AF-PRS gene signature and closely related LUAD nearest centroid classifier were originally developed using patients with a primary diagnosis of LUAD, it was applied to the overall population of NS-NSCLC included in the current study, the majority of which were LUAD.

### Statistics

The signatures and individual genes were presented based on values generated using log 2 median-centered expression values of genes making up different signatures and individual genes. Boxplots showing individual immune activation signatures or individual gene expression levels were also created and pairwise comparisons were conducted with p-values displayed when Kruskal-Wallis Test p-values were <0.05). Heatmaps and box plots were generated using R program version 3.5.3. Box plots show lower quartile, median and upper quartile expression data. Plot whiskers show the full distribution of the expression data.

OS and PFS analyses were conducted using Cox-Proportional Hazards (CPH) model with right censored endpoints. Associations between response to treatment and genomic markers were investigated using the Fisher's exact or Kruskal-Wallis rank tests for quantitative and qualitative markers. Association between response to treatment and clinical characteristics were evaluated using Fisher's exact test. Multivariable logistic regression models were used to test whether molecular subtype predicts response to treatment when adjusting for various genomic markers. All statistical analyses were conducted using R 3.6 software (cran.R-project.org)

### Results

Baseline demographics and disease status, which were abstracted from relevant patient records, are presented in Table 8 and include a comparison of those who were AF-PRS(+) and AF-PRS(−).

TABLE 8
Baseline demographics and disease status
of the study population by AF-PRS status.

| Baseline | All | | AF-PRS(+) | | AF-PRS(−) | | |
|---|---|---|---|---|---|---|---|
| Characteristics | (n = 95) | | (n = 50; 53%) | | (n = 45; 47%) | | p** |
| Gender, n(%*) | | | | | | | |
| Female | 47 | (49%) | 28 | (56%) | 19 | (42%) | 0.22 |
| Male | 48 | (51%) | 22 | (44%) | 26 | (58%) | |
| NA | 0 | | 0 | | 0 | | |
| Race, n(%) | | | | | | | |
| White | 82 | (86%) | 43 | (86%) | 39 | (87%) | 0.65 |
| African American | 12 | (13%) | 7 | (14%) | 5 | (11%) | |
| other | 1 | (1%) | 0 | (0%) | 1 | (2%) | |
| Age (years) | | | | | | | |
| median | 68 | | 70 | | 66 | | 0.28 |
| Age Category [min, max] | | | | | | | |
| [43, 66) | 42 | (44%) | 20 | (40%) | 22 | (49%) | 0.41 |
| [66, 90] | 53 | (56%) | 30 | (60%) | 23 | (51%) | |
| NA | 0 | | 0 | | 0 | | |
| History of Smoking, n(%) | | | | | | | |
| yes | 85 | (89%) | 44 | (88%) | 41 | (91%) | 0.74 |
| no | 10 | (11%) | 6 | (12%) | 4 | (9%) | |
| NSCLC Dx, n(%) | | | | | | | |
| adeno | 84 | (88%) | 45 | (90%) | 39 | (87%) | 0.75 |
| other | 11 | (12%) | 5 | (10%) | 6 | (13%) | |
| T at Dx, n(%) | | | | | | | |
| T1 | 17 | (35%) | 9 | (31%) | 8 | (42%) | 0.83 |
| T2 | 14 | (29%) | 9 | (31%) | 5 | (26%) | |
| T3 | 12 | (25%) | 7 | (24%) | 5 | (26%) | |
| T4 | 5 | (10%) | 4 | (14%) | 1 | (5%) | |
| NA | 47 | | 21 | | 26 | | |
| N at Dx, n(%) | | | | | | | |
| N0 | 13 | (28%) | 9 | (31%) | 4 | (24%) | 0.06 |
| N1 | 15 | (33%) | 12 | (41%) | 3 | (18%) | |
| N2 | 12 | (26%) | 7 | (24%) | 5 | (29%) | |
| N3 | 6 | (13%) | 1 | (3%) | 5 | (29%) | |
| NA | 49 | | 21 | | 28 | | |
| M at Dx, n(%) | | | | | | | |
| M0 | 28 | (61%) | 18 | (72%) | 10 | (48%) | 0.13 |
| M1 | 18 | (39%) | 7 | (28%) | 11 | (52%) | |
| NA | 49 | | 25 | | 24 | | |
| Stage at Dx, n(%) | | | | | | | |
| I | 2 | (2%) | 0 | (0%) | 2 | (10%) | 0.022 |
| II | 19 | (38%) | 15 | (52%) | 4 | (19%) | |
| III | 12 | (24%) | 8 | (28%) | 4 | (19%) | |
| IV | 17 | (34%) | 7 | (23%) | 11 | (52%) | |
| NA | 45 | (47%) | 20 | | 24 | | |
| Stage at Treatment, n(%) | | | | | | | |
| I-III | 26 | (27%) | 19 | (38%) | 7 | (16%) | 0.021 |
| IV | 69 | (73%) | 31 | (62%) | 38 | (84%) | |
| Molecular Subtype, n(%) | — | | — | | — | | — |
| bronchioid | 50 | (53%) | 50 | (100%) | 0 | (0%) | |
| magnoid | 27 | (28%) | 0 | (0%) | 27 | (60%) | |
| squamoid | 18 | (19%) | 0 | (0%) | 18 | (40%) | |
| AF-PRS Status, n(%) | | | | | | | |
| + | 50 | (53%) | 50 | (100%) | 0 | (0%) | |
| − | 45 | (47%) | 0 | (0%) | 45 | (100%) | |
| NA | 0 | | 0 | | 0 | | |
| PDL1 Status, n(%) | | | | | | | |
| + | 39 | (58%) | 21 | (62%) | 18 | (55%) | 0.62 |
| − | 28 | (42%) | 13 | (38%) | 15 | (45%) | |
| NA | 28 | | 16 | | 12 | | |
| PD-L1 Staining, n(%) | | | | | | | |
| <1% | 28 | (42%) | 13 | (38%) | 15 | (45%) | 1 |
| 1-50% | 26 | (39%) | 13 | (38%) | 13 | (39%) | |
| >50% | 13 | (19%) | 8 | (24%) | 5 | (15%) | |

| | | | |
|---|---|---|---|
| NA | 28 | 16 | 12 |

*calculated as the percentage of the overall group with data available
**P-value comparing overall difference between AF-PRS(+) and AF-PRS(−) patients using Fisher Exact test except the Age (years) median.
NA = not available

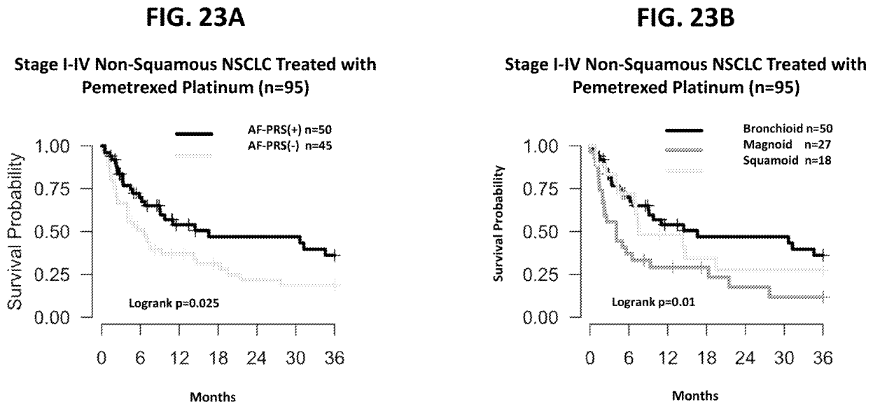

Consistent with other findings (Fennel et al, 2014), a majority of the NS-NSCLC patients had a primary diagnosis of adenocarcinoma (880%) with the remainder diagnoses that included NSCLC NOS, poorly differentiated NSCLC, undifferentiated large cell carcinoma etc. Overall, patients were well balanced by AF-PRS status for demographics. Fifty-three percent of patients were AF-PRS (+) (bronchioid molecular subtype) with the remaining 4700 AF-PRS(−) (magnoid/squamoid molecular subtype). There were no significant differences in demographics by AF-PRS status, however, patients who were AF-PRS(+) generally had a lower stage disease, including a trend towards decreased node involvement at diagnosis, as significant differences in overall stage at diagnosis and at treatment, including those who were Stage I-III vs. IV at treatment. Therefore, in the survival and clinical response analyses described in FIGS. 23A-B and 24, the subset of patients who were Stage I-III at time of treatment were evaluated independent of those who were Stage IV. The PMX-PDC patients included in the current study were treated both prior to and after the introduction of anti-PD-(L)1 therapy use, therefore only 71% of the patients had PD-L1 status recorded, of which just over half (58%) were PD-L1 (+) (≥1% TPS) which is consistent with other investigations (Gandhi et al, N Engl J Med. 2018).

The median duration of follow-up for this retrospective analysis was 43.7 months (37.9-63.8) for the overall cohort, and 40.9 months (14.5-55.9) and 50.7 (41.1-NR) for AF-PRS(+) and AF-PRS(−), respectively, which exceeded the median duration of follow-up for Phase 3 studies that included the evaluation of PMX-PDC (10.5-12.5 months (Paz-Ares et al, J Clin Oncol. 2013; Reck et al, J Clin Oncol. 2019; Gandi et al, N Engl J Med. 2018). Because median duration of follow-up for the overall cohort was less than 4 years, administrative censoring was performed at 3 years as reflected in the survival curves below.

Clinical outcomes following treatment with PMX-PDC for the overall study population (n=95) as well as those who were AF-PRS(+) and AF-PRS(−) are summarized Table 9.

TABLE 9
Clinical treatment outcomes by AF-PRS status

| | All | | AF-PRS(+) | | AF-PRS(−) | |
|---|---|---|---|---|---|---|
| Outcomes | (n = 95) | | (n = 50) | | (n = 45) | |
| Best response, n (%) | | | | | | |
| CR | 14 | (15%) | 11 | (22%) | 3 | (7%) |
| PR | 33 | (37%) | 12 | (24%) | 21 | (51%) |
| SD | 25 | (28%) | 18 | (37%) | 7 | (17%) |
| PD | 18 | (20%) | 8 | (16%) | 10 | (24%) |
| NA | 5 | | 1 | | 4 | |
| ORR, n(%) | 47 | (52) | 23 | (47) | 24 | (58) |
| Clinical benefit*, n(%) | | | | | | |
| Yes | 72 | (80%) | 41 | (84%) | 31 | (77%) |
| Median PFS#, mos (95% CI) | 9.07 | (6.54-19.5) | 16.57 | (8.98-NR) | 6.54 | (4.01-14.7) |
| Rate of PFS at 6 months, (95% CI) | 60.7% | (51.4-71.6) | 69.9% | (57.8-84.5) | 50.9% | (38.1-67.9) |
| Rate of PFS at 12 months, (95% CI) | 45.7% | (36.2-57.6) | 53.9% | (40.7-71.6) | 36.9% | (25.0-54.4) |
| Median OS\hat{ }, mos (95% CI) | 24.2 | (15.3-NR) | 24.59 | (15.3-NR) | 24.23 | (8.4-NR) |
| Rate of OS at 6 months, n(%) | 74.5% | (66.0-84.1) | 82.6% | (72.3-94.4) | 66.0% | (53.3-81.6) |
| Rate of OS at 1 year, n(%) | 63.3% | (53.8-74.4) | 67.5% | (54.7-83.3) | 58.5% | (45.5-75.3) |

*Clinical benefit defined as CR + PR + SD
**P-value comparing overall difference between AF-PRS(+) and AF-PRS(−) patients using Fisher Exact test
#PFS defined as time from PMX-PDC treatment initiation to progression or death and was also the duration of response
\hat{ }OS defined as time from PMX-PDC treatment initiation to death.
NR = not reached

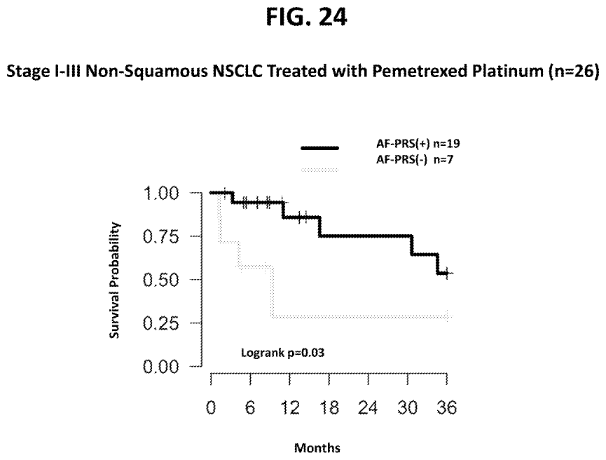

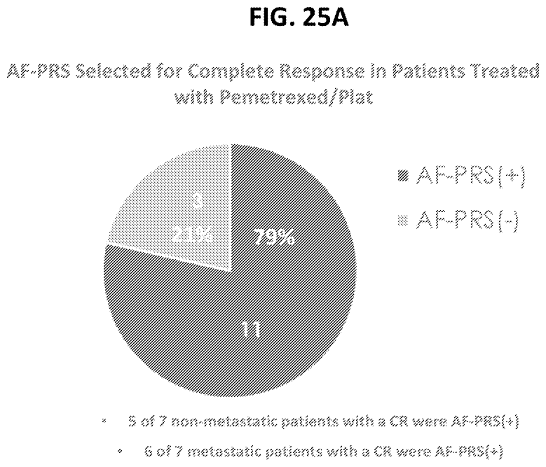

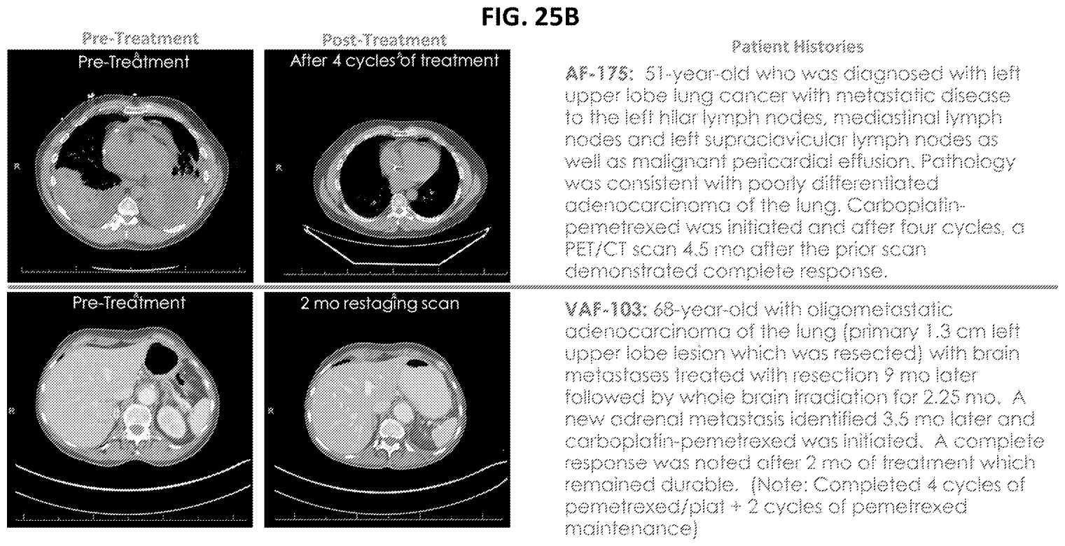

A significant difference in the clinical response was observed between AF-PRS(+) vs. AF-PRS(−) patients (p=0.009), with a greater proportion of AF-PRS(+) patients having a CR to PMX-PDC (described in further detail in FIG. 25A). Also, extended PFS (˜2.5× longer) was noted in AF-PRS(+) vs. AF-PRS(−) patients, which was consistent with the significant survival difference noted in FIG. 23A. The rates of PFS in the AF-PRS(+) patients at 6 and 12 months were numerically greater than the rates observed in the AF-PRS(−) patients at these respective timepoints. While the rate of OS at 6 months was numerically greater in those who were AF-PRS(+), the median OS was similar between AF-PRS(+) and (−) patients (Table 9). The Kaplan Meier survival curves for the overall cohort were significantly different based upon AF-PRS status (FIG. 23A) or when split by the associated LUAD subtyper (FIG. 23B). As noted above, since there was a difference by AF-PRS status in the relative proportion of patients who were Stage I-III vs. Stage IV at time of treatment, Stage I-III patients were evaluated independently (FIG. 24). This resulted in a similar, if not stronger, separation of the survival curves when focused on the Stage I-III patients despite the reduced number of patients. It should be noted that while FIGS. 23A-B and 24 include those who were Stage I-III at treatment, only 2 patients in the entire cohort were Stage I at diagnosis.

While overall response rate (ORR) and the clinical response rate (CR+PR) were similar between AF-PRS(+) and (−) patients, further evaluation of those with a complete response (CR) revealed that AF-PRS positivity appears to select for patients with a CR (Table 9; FIG. 25A). For example, while the overall CR rate was 15%, 22% of the AF-PRS(+) patients and 7% of the AF-PRS(−) patients had a CR. For the 14 of 95 (15%) patients with a CR to pemetrexed/platinum, a vast majority (11 of 14 (79%)) were AF-PRS(+), including 5 of 7 and 6 of 7 who were Stage I-III and Stage IV, respectively, at the time of treatment. Representative scans, along with detailed patient histories, are provided for two of the AF-PRS(+) patients who were Stage IV at the time of treatment (FIG. 25B).

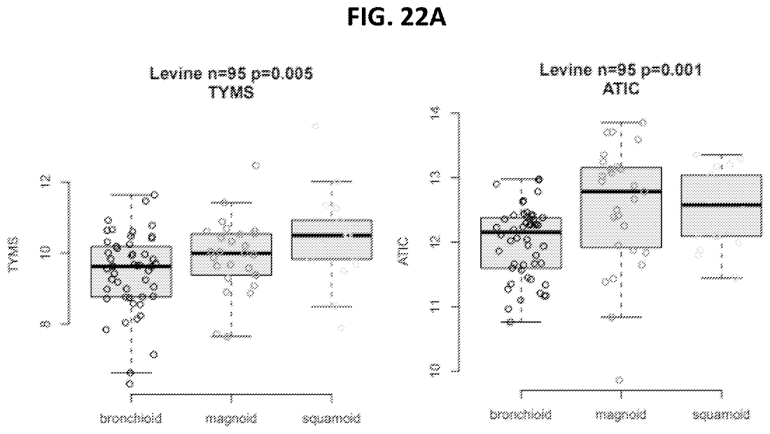

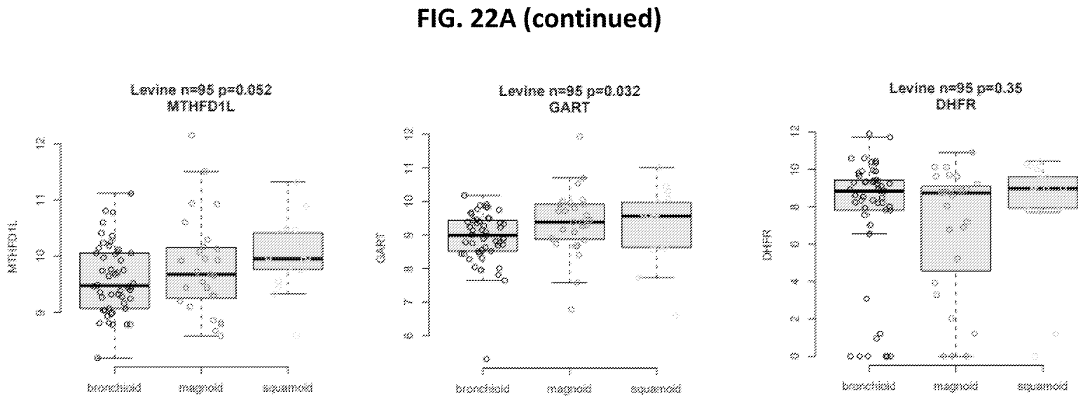

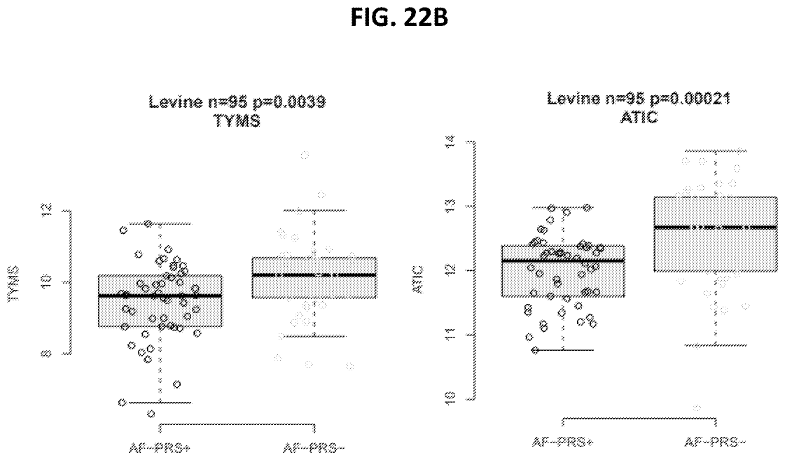

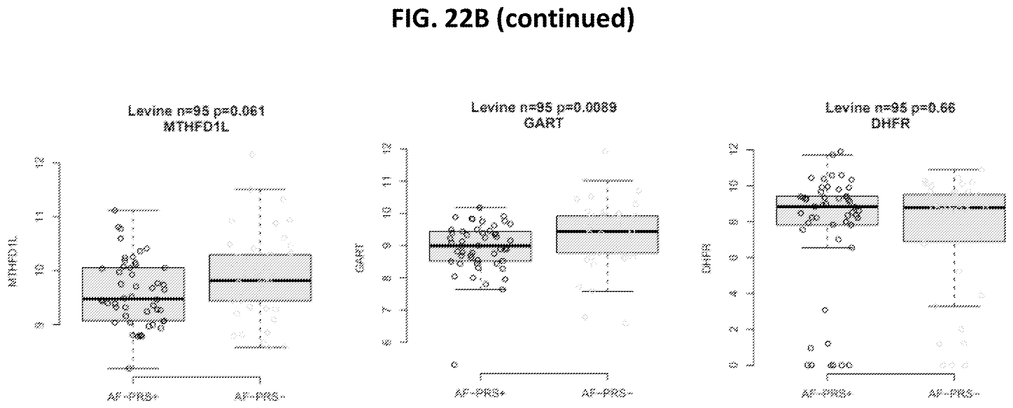

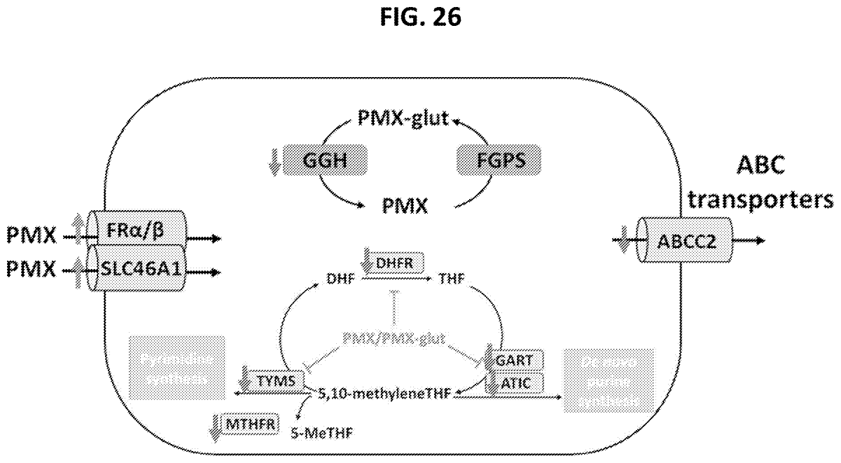

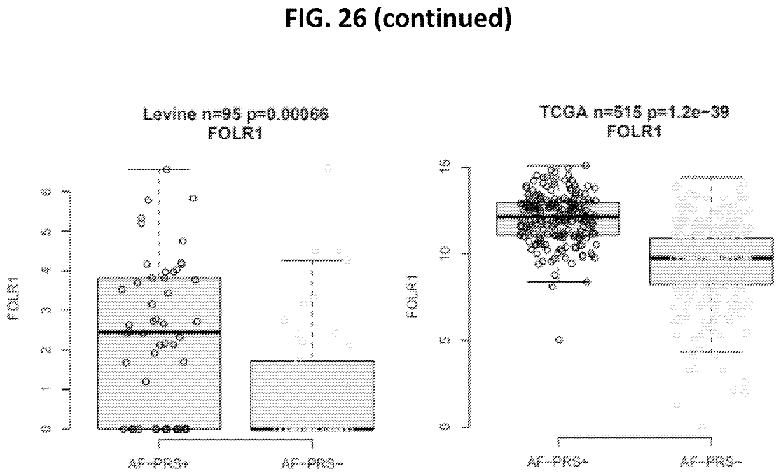

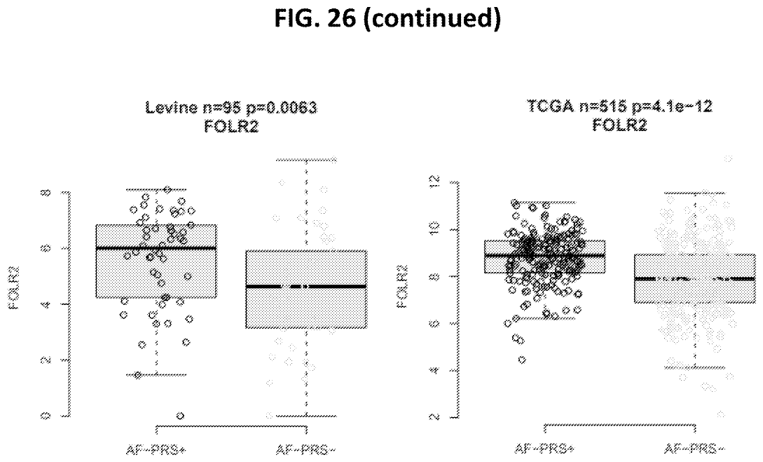

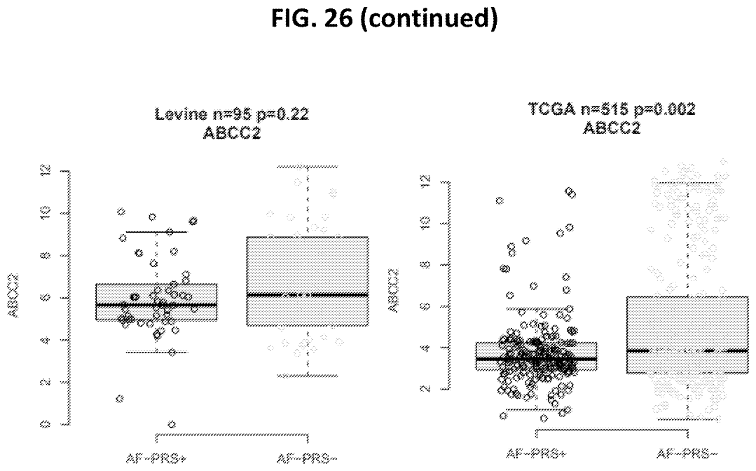

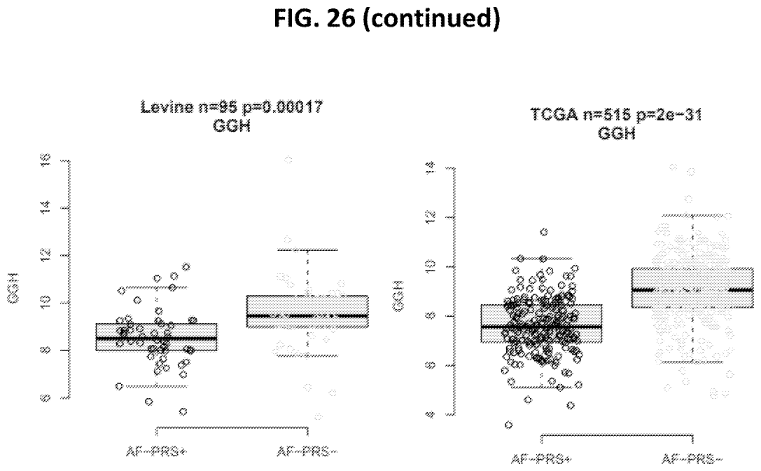

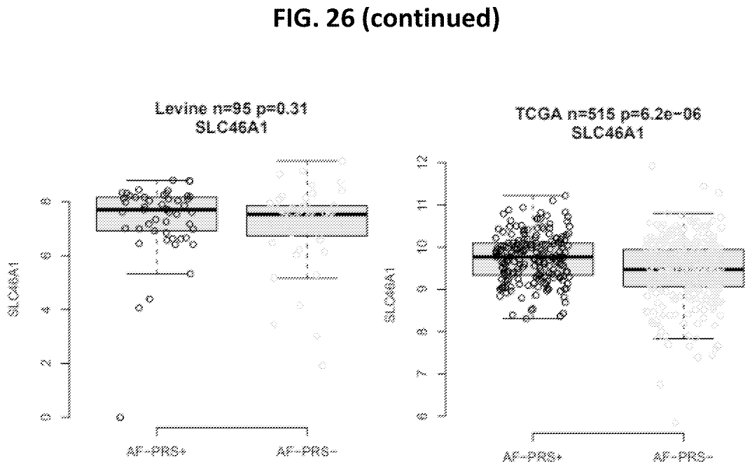

Differential gene expression of pemetrexed target genes as well as genes for transporters involved in its cellular influx/efflux was evaluated to gain insight into the molecular mechanisms that may contribute to the pemetrexed differential responses observed based upon AF-PRS status. Pemetrexed/antifolate target genes of interest included ATIC, DHFR, GART, MTHFD1L, TYMS and GART and their relative expression levels by AF-PRS status/LUAD subtype are presented in FIG. 22B, respectively as well as genes associated with pemetrexed/antifolate metabolism (FIG. 26; FOLR1, FOLR2, ABCC2, GGH and SLC46A1). Expression of TYMS, ATIC and GART was significantly lower in AF-PRS(+) relative to AF-PRS(−) samples in both this Example (i.e., the Piedmont Study) and TCGA LUAD cohorts and MTHFD1L and DHFR was expression was similarly decreased in the larger TCGA LUAD cohort. Similar differences were noted when split by LUAD subtype (FIG. 22A).

To further elucidate potential biological underpinnings that may contribute to pemetrexed response in patients with AF-PRS(+) tumors, genes associated with cellular trafficking and detoxification of pemetrexed were also interrogated (FIG. 26). Significantly higher expression of folate receptor genes (FOLR1 and FOLR2) in AF-PRS(+) tumors were observed in both this Example's (i.e., the Piedmont Study) and TCGA LUAD cohorts. ABCC2, which is responsible for folate efflux, was significantly lower AF-PRS(+) samples from the larger TCGA LUAD cohort. Similarly, lower expression of gamma-glutamyl hydrolase (GGH) expression levels were observed in AF-PRS(+) samples. While several of the genes noted above (GGH, TYMS, FOLR2 and FOLR1) were included in the original 506 gene subtyper developed by Wilkerson et al PLoS One 2012 with relative subtype associations, the current study mapped their activities to the metabolism of pemetrexed in the context of preferential PMX-PDC response in AF-PRS(+)/bronchioid tumors.

### REFERENCES

Baxevanos P. Mountzios G. Novel chemotherapy regimens for advanced lung cancer: have we reached a plateau? Ann Transl Med 2018 April; 6(8):139.
Scagliotti G V et al., Phase III study comparing cisplatin plus gemcitabine with cisplatin plus pemetrexed in chemotherapy-naive patients with advanced-stage non-small-cell lung cancer. J Clin Oncol. 2008 Jul. 20; 26(21):3543-51. doi: 10.1200/JCO.2007.15.0375. Epub 2008 May 27.
Sun J M, Han J, Ahn J S, Park K, Ahn M J. Significance of thymidylate synthase and thyroid transcription factor 1 expression in patients with nonsquamous non-small cell lung cancer treated with pemetrexed-based chemotherapy. J Thorac Oncol 2011; 6:1392-1399.
Chen C Y, Chang Y L, Shih J Y, et al. Thymidylate synthase and dihydrofolate reductase expression in non-small cell lung carcinoma: the association with treatment efficacy of pemetrexed. Lung Cancer 2011; 74:132-138.
Sigmond J, Backus H H, Wouters D, Temmink O H, Jansen G, Peters G J. Induction of resistance to the multitargeted antifolate Pemetrexed (ALIMTA) in WiDr human colon cancer cells is associated with thymidylate synthase overexpression. Biochem Pharmacol 2003; 66:431-438.
Takezawa K, Okamoto I, Okamoto W, et al. Thymidylate synthase as a determinant of pemetrexed sensitivity in non-small cell lung cancer. Br J Cancer 2011; 104:1594-1601.
Hayes D N et al., Gene expression profiling reveals reproducible human lung adenocarcinoma subtypes in multiple independent patient cohorts. J Clin Oncol. 2006 November 124(31):5079-90.
Wilkerson M D et al., Differential pathogenesis of lung adenocarcinoma subtypes involving sequence mutations, copy number, chromosomal instability, and methylation. PLoS One. 2012; 7(5):e36530, doi: 10.1371/journal.pone.0036530. Epub 2012 May 10.
Nicolson M C et al., Thymidylate synthase expression and outcome of patients receiving pemetrexed for advanced nonsquamous non-small-cell lung cancer in a prospective blinded assessment phase II clinical trial. J Thorac Oncol. 2013 July; 8(7):930-9.
Fennell D A, Myrand S P, Nguyen T S, Ferry D, Kerr K M, Maxwell P, Moore S D, Visseren-Grul C, Das M, Nicolson M C. Association between gene expression profiles and clinical outcome of pemetrexed-based treatment in patients with advanced non-squamous non-small cell lung cancer: exploratory results from a phase II study. PLoS One. 2014 Sep. 24; 9(9):e107455. Dobin A, Davis C A, Schlesinger F, Drenkow J, Zaleski C, Jha S, et al. STAR: Ultrafast universal RNA-seq aligner. Bioinformatics. 2013; 29:15-21.
Li B, Dewey C N. RSEM: Accurate transcript quantification from RNA-Seq data with or without a reference genome. BMC Bioinformatics. 2011; 12.
Love M I, Huber W, Anders S. Moderated estimation of fold change and dispersion for RNA-seq data with DESeq2. Genome Biol. 2014; 15.
Gandhi L et al., Pembrolizumab plus Chemotherapy in Metastatic Non-Small-Cell Lung Cancer. N Engl J Med. 2018 May 31; 378(22):2078-2092. doi: 10.1056/NEJMoa1801005. Epub 2018 Apr. 16.
Paz-Ares L G et al., PARAMOUNT: Final overall survival results of the phase HI study of maintenance pemetrexed versus placebo immediately after induction treatment with pemetrexed plus cisplatin for advanced nonsquamous non-small-cell lung cancer. J Clin Oncol. 2013 Aug. 10; 31(23):2895-902. doi: 10.1200/JCO.2012.47.1102. Epub 2013 Jul. 8.
Reck M et al., Updated Analysis of KEYNOTE-024: Pembrolizumab Versus Platinum-Based Chemotherapy for Advanced Non-Small-Cell Lung Cancer With PD-L1 Tumor Proportion Score of 50% or Greater. J Clin Oncol. 2019 Mar. 1; 37(7):537-546.

### Further Numbered Embodiments of the Disclosure

Other subject matter contemplated by the present disclosure is set out in the following numbered embodiments:

1. A method of detecting a biomarker in a sample obtained from a patient suffering from cancer, the method comprising measuring the nucleic acid expression level of a plurality of biomarkers selected from Table 1 using an amplification, hybridization and/or sequencing assay.

2. The method of embodiment 1, wherein the patient was previously diagnosed with a cancer selected from bladder cancer, breast cancer, pancreatic adenocarcinoma, lung adenocarcinoma, lung squamous cell carcinoma, and head and neck adenocarcinoma.

3. The method of embodiment 1, wherein the amplification, hybridization and/or sequencing assay comprises performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays, Northern blotting, or any other equivalent nucleic acid expression detection techniques.

4. The method of embodiment 3, wherein the nucleic acid expression level is detected by performing qRT-PCR.

5. The method of embodiment 4, wherein the detection of the nucleic acid expression level comprises using at least one pair of oligonucleotide primers per each of the plurality of biomarkers selected from Table 1.

6. The method of any one of the above embodiments, wherein the sample is a formalin-fixed, paraffin-embedded (FFPE) tissue sample, fresh or a frozen tissue sample, an exosome, wash fluids, cell pellets, or a bodily fluid obtained from the patient.

7. The method of embodiment 6, wherein the bodily fluid is blood or fractions thereof, urine, saliva, or sputum.

8. The method of any one of the above embodiments, wherein the plurality of biomarkers comprises at least 8 biomarkers, at least 16 biomarkers, at least 24 biomarkers, at least 32 biomarkers, at least 40 biomarkers or at least 48 biomarkers of Table 1.

9. The method of any one of embodiments 1-7, wherein the plurality of biomarkers selected from Table 1 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 1.

10. The method of any one of embodiments 1-7, wherein the plurality of biomarkers selected from Table 1 comprise flgf, ctsh, sctr, cyp4b1, gpr116, adh1b, cbx7, hlf cep55, tpx2, bub1b, kif4a, ccnb2, kif14, melk, kif11 or any combination thereof.

11. The method of any one of embodiments 1-7, wherein the plurality of biomarkers of Table 1 comprise fgl1, pbk, hspd1, tdg, prc1, dusp4, gtpbp4, zwint, tlr2, cd74, hla-dpb1, hla-dpa1, hla-dra, itgb2, fas, hla-drb1, plau, gbp1, dse, ccdc109b, tgfbi, cxcl10, lgals1, tubb6, gjb1, rap1gap, cacna2d2, selenbp1, tfcp2l1, sorbs2, unc13b, tacc2 or any combination thereof.

12. The method of any one of the above embodiments, wherein the plurality of biomarkers comprises all the classifier biomarkers of Table 1.

13. A method of detecting a biomarker in a sample obtained from a patient suffering from cancer, the method consisting essentially of measuring the nucleic acid expression level of a plurality of biomarkers selected from Table 1 using an amplification, hybridization and/or sequencing assay.

14. The method of embodiment 13, wherein the patient was previously diagnosed with a cancer selected from bladder cancer, breast cancer, pancreatic adenocarcinoma, lung adenocarcinoma, lung squamous cell carcinoma, and head and neck adenocarcinoma.

15. The method of embodiment 13 or 14, wherein the amplification, hybridization and/or sequencing assay comprises performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays, Northern blotting, or any other equivalent gene expression detection techniques.

16. The method of embodiment 15, wherein the nucleic acid expression level is detected by performing qRT-PCR.

17. The method of embodiment 16, wherein the detection of the nucleic acid expression level comprises using at least one pair of oligonucleotide primers per each of the plurality of biomarkers selected from Table 1.

18. The method of any one of embodiments 13-17, wherein the sample is a formalin-fixed, paraffin-embedded (FFPE) tissue sample, fresh or a frozen tissue sample, an exosome, wash fluids, cell pellets, or a bodily fluid obtained from the patient.

19. The method of embodiment 18, wherein the bodily fluid is blood or fractions thereof, urine, saliva, or sputum.

20. The method of any one of embodiments 13-19, wherein the plurality of biomarkers consists essentially of at least 8 biomarkers, at least 16 biomarkers, at least 24 biomarkers, at least 32 biomarkers, at least 40 biomarkers or at least 48 biomarkers of Table 1.

21. The method of any one of embodiments 13-19, wherein the plurality of biomarkers selected from Table 1 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 1.

22. The method of any one of embodiments 13-19, wherein the plurality of biomarkers selected from Table 1 comprise flgf, ctsh, sctr, cyp4b1, gpr116, adh1b, cbx7, hlf cep55, tpx2, bub1b, kif4a, ccnb2, kif14, melk, kif11 or any combination thereof.

23. The method of any one of embodiments 13-19, wherein the plurality of biomarkers of Table 1 comprise fgl1, pbk, hspd1, tdg, prc1, dusp4, gtpbp4, zwint, tlr2, cd74, hla-dpb1, hla-dpa1, hla-dra, itgb2, fas, hla-drb1, plau, gbp1, dse, ccdc109b, tgfbi, cxcl10, lgals1, tubb6, gjb1, rap1gap, cacna2d2, selenbp1, tfcp2l1, sorbs2, unc13b, tacc2 or any combination thereof.

24. The method of any one of embodiments 13-19, wherein the plurality of biomarkers consists essentially of all the biomarkers of Table 1.

25. A method of detecting a biomarker in a sample obtained from a patient suffering from cancer, the method consisting of measuring the nucleic acid expression level of a plurality of biomarkers selected from Table 1 using an amplification, hybridization and/or sequencing assay.

26. The method of embodiment 25, wherein the patient was previously diagnosed with a cancer selected from bladder cancer, breast cancer, pancreatic adenocarcinoma, lung adenocarcinoma, lung squamous cell carcinoma, and head and neck adenocarcinoma.

27. The method of embodiment 25 or 26, wherein the amplification, hybridization and/or sequencing assay comprises performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays, Northern blotting, or any other equivalent gene expression detection techniques.

28. The method of embodiment 27, wherein the nucleic acid expression level is detected by performing qRT-PCR.

29. The method of embodiment 28, wherein the detection of the nucleic acid expression level comprises using at least one pair of oligonucleotide primers per each of the plurality of biomarkers selected from Table 1.

30. The method of any one of embodiments 25-29, wherein the sample is a formalin-fixed, paraffin-embedded (FFPE) tissue sample, fresh or a frozen tissue sample, an exosome, wash fluids, cell pellets, or a bodily fluid obtained from the patient.

31. The method of embodiment 30, wherein the bodily fluid is blood or fractions thereof, urine, saliva, or sputum.

32. The method of any one of embodiments 25-31, wherein the plurality of biomarkers consists of at least 8 biomarkers, at least 16 biomarkers, at least 24 biomarkers, at least 32 biomarkers, at least 40 biomarkers or at least 48 biomarkers of Table 1.

33. The method of any one of embodiments 25-31, wherein the plurality of biomarkers selected from Table 1 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 1.

34. The method of any one of embodiments 25-31, wherein the plurality of biomarkers selected from Table 1 comprise flgf, ctsh, sctr, cyp4b1, gpr116, adh1b, cbx7, hlf cep55, tpx2, bub1b, kif4a, ccnb2, kif14, melk, kif11_or any combination thereof.

35. The method of any one of embodiments 25-31, wherein the plurality of biomarkers of Table 1 comprise fgl1, pbk, hspd1, tdg, prc1, dusp4, gtpbp4, zwint, tlr2, cd74, hla-dpb1, hla-dpa1, hla-dra, itgb2, fas, hla-drb1, plau, gbp1, dse, ccdc109b, tgfbi, cxcl10, lgals1, tubb6, gjb1, rap1gap, cacna2d2, selenbp1, tfcp2l1, sorbs2, unc13b, tacc2 or any combination thereof.

36. The method of any one of embodiments 25-31, wherein the plurality of biomarkers comprises, consists essentially of or consists of all the biomarkers of Table 1.

37. A method of determining whether a patient suffering from cancer is likely to respond to treatment with an antifolate agent, the method comprising, determining an antifolate predictive response signature of a sample obtained from a patient suffering from cancer; and based on the antifolate predictive response signature, assessing whether the patient is likely to respond to treatment with an antifolate agent, wherein a positive antifolate predictive response signature predicts that the patient is likely to respond to the treatment with an antifolate agent.

38. A method for selecting a patient suffering from cancer for an antifolate agent, the method comprising, determining an antifolate predictive response signature of a sample obtained from a patient suffering from cancer; and selecting the patient for treatment with an antifolate agent if the antifolate response signature is positive.

39. The method of embodiment 37 or 38, wherein the anti-folate agent is selected from pemetrexed, methotrexate, trimetrexate, lometrexol, raltitrexed and nolatrexed.

40. The method of embodiment 39, wherein the antifolate agent is pemetrexed.

41. The method of embodiment 39, wherein the antifolate agent is raltitrexed.

42. The method of any one of embodiments 37-41, wherein the cancer the patient is suffering from is selected from bladder cancer, breast cancer, pancreatic adenocarcinoma, lung adenocarcinoma, lung squamous cell carcinoma, and head and neck adenocarcinoma.

43. The method of any one of embodiments 37-42, wherein the sample is a formalin-fixed, paraffin-embedded (FFPE) tissue sample, fresh or a frozen tissue sample, an exosome, or a bodily fluid obtained from the patient.

44. The method of embodiment 43, wherein the bodily fluid is blood or fractions thereof, urine, saliva, or sputum.

45. The method of any one of embodiments 37-44, wherein the determining the antifolate predictive response signature of the sample obtained from the patient suffering from cancer comprises determining expression levels of a plurality of classifier biomarkers.

46. The method of embodiment 45, wherein the determining the expression levels of the plurality of classifier biomarkers is at a nucleic acid level by performing RNA sequencing, reverse transcriptase polymerase chain reaction (RT-PCR) or hybridization-based analyses.

47. The method of embodiment 45 or 46, wherein the plurality of classifier biomarkers for determining the antifolate predictive response signature is selected from Table 1.

48. The method of embodiment 47, wherein the RT-PCR is quantitative real time reverse transcriptase polymerase chain reaction (qRT-PCR).

49. The method of embodiment 48, wherein the RT-PCR is performed with primers specific to the classifier biomarkers selected from the plurality of classifier biomarkers of Table 1.

50. The method of any one of embodiments 47-49, further comprising comparing the detected levels of expression of the plurality of classifier biomarkers of Table 1 to the expression of the plurality of classifier biomarkers of Table 1 in at least one sample training set(s), wherein the at least one sample training set comprises expression data of the plurality of classifier biomarkers of Table 1 from a reference adenocarcinoma TRU (bronchioid) sample, expression data of the plurality of classifier biomarkers of Table 1 from a reference adenocarcinoma PP (magnoid) sample, expression data of the plurality of classifier biomarkers of Table 1 from a reference adenocarcinoma PI (squamoid) sample, or a combination thereof; and classifying the sample as TRU, PP, or PI based on the results of the comparing step.

51. The method of embodiment 50, wherein the comparing step comprises applying a statistical algorithm which comprises determining a correlation between the expression data obtained from the sample and the expression data from the at least one training set(s); and classifying the sample as a TRU, PP, or PI subtype based on the results of the statistical algorithm.

52. The method of embodiment 50 or 51, wherein the TRU subtype is indicative of a positive antifolate predictive response signature, wherein the positive antifolate predictive response signature selects the patient for treatment with an antifolate agent.

53. The method of any one of embodiments 47-52, wherein the plurality of classifier biomarkers comprises at least 8 biomarker nucleic acids, at least 16 biomarker nucleic acids, at least 24 biomarker nucleic acids, at least 32 biomarker nucleic acids, at least 140 biomarker nucleic acids or all 48 biomarker nucleic acids of Table 1.

54. The method of any one of embodiments 47-52, wherein the plurality of classifier biomarkers selected from Table 1 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the classifier biomarkers from Table 1.

55. The method of any one of embodiments 47-52, wherein the plurality of classifier biomarkers selected from Table 1 comprise flgf, ctsh, sctr, cyp4b1, gpr116, adh1b, cbx7, hlf cep55, tpx2, bub1b, kif4a, ccnb2, kif14, melk, kif11 or any combination thereof.

56. The method of any one of embodiments 47-52, wherein the plurality of classifier biomarkers of Table 1 comprise fgl1, pbk, hspd1, tdg, prc1, dusp4, gtpbp4, zwint, tlr2, cd74, hla-dpb1, hla-dpa1, hla-dra, itgb2, fas, hla-drb1, plau, gbp1, dse, ccdc109b, tgfbi, cxcl10, lgals1, tubb6, gjb1, rap1gap, cacna2d2, selenbp1, tfcp2l1, sorbs2, unc13b, tacc2 or any combination thereof.

57. The method of any one of embodiments 37-56, wherein the method further comprises determining the expression level of one or more anti-folate drug targets in the sample obtained from the patient.

58. The method of embodiment 57, wherein the one or more anti-folate drug targets is selected from dhfr, gart, tyms, atic, or mthfd1l genes.

59. The method of any one of embodiments 37-58, wherein the method further comprises determining a tumor mutational burden of the tumor sample obtained from the patient.

60. The method of any one of embodiments 37-58, wherein the method further comprises determining a proliferation signature of the tumor sample obtained from the patient.

61. The method of embodiment 60, wherein the determining the proliferation signature in the tumor sample obtained from a patient comprises measuring a nucleic acid expression level in the sample of at least five classifier genes from a plurality of classifier genes, wherein the plurality of classifier genes consists of only targeting protein for Xklp2 (TPX2), discs large homolog associated protein 5 (DLGAP5), Holliday junction recognition protein (HJURP), kinesin family member 4A (KIF4A), kinesin family member 2C (KIF2C), polo like kinase 1 (PLK1), maternal embryonic leucine zipper kinase (MELK), Cyclin B2 (CCNB2), budding uninhibited by benzimidazoles 1 (BUB1), kinesin family member 23 (KIF23), ubiquitin conjugating enzyme E2 C (UBE2C), kinesin family member 20A (KIF20A), trophinin associated protein (TROAP), aurora kinase B (AURKB), ribonucleotide reductase regulatory subunit M2 (RRM2), MYB proto-oncogene like 2 (MYBL2), antigen KI-67 (MK167), cell division cycle 20 (CDC20), centrosomal protein 55 (CEP55), topoisomerase 2-alpha (TOP2A), baculoviral IAP repeat containing 5 (BIRC5), abnormal spindle microtubule assembly (ASPM), extra spindle pole bodies like 1, separase (ESPL1), kinesin family member 18B (KIF18B), IQ motif containing GTPase activating protein 3 (IQGAP3), and effector cell protease receptor-1 (EPR1), wherein the nucleic acid expression level of the at least five classifier genes represents a proliferation signature.

62. The method of embodiment 61, wherein the nucleic acid expression level is measured using an amplification, sequencing or hybridization assay.

63. The method of embodiment 62, wherein the amplification, hybridization and/or sequencing assay comprises performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays, Northern blotting, nCounter DX Analysis System or any other equivalent gene expression detection techniques.

64. The method of embodiment 63, wherein the expression level is detected by performing RNA-seq.

65. The method of embodiment 61, wherein the measuring the nucleic acid expression level is for at least 10, 15, 20 or 25 classifier genes from the plurality of classifier genes.

66. The method of embodiment 61, wherein the measuring the nucleic acid expression level is for all of the classifier genes from the plurality of classifier genes.

67. The method of embodiment 61, further comprising determining a proliferation score, wherein the determining the proliferation score comprises determining a mean nucleic acid expression level for the at least five classifier biomarkers from the plurality of classifier biomarkers.

68. The method of any one of embodiments 61-67, further comprising determining a level and/or activity of at least one additional marker involved in cell proliferation and mitosis.

69. The method of embodiment 68, wherein the at least one additional marker is Ki67 or CD31.

70. A method of treating cancer in a patient, the method comprising: measuring the expression level of a plurality of classifier biomarkers in a sample obtained from a patient suffering from cancer, wherein the plurality of classifier biomarkers are selected from a set of classifier biomarkers listed in Table 1, wherein the measured expression levels of the plurality of classifier biomarkers provide an antifolate predictive response signature for the sample; and
administering an antifolate agent based on presence of a positive antifolate predictive response signature.

71. The method of embodiment 70, wherein the anti-folate agent is selected from pemetrexed, methotrexate, trimetrexate, lometrexol, raltitrexed and nolatrexed.

72. The method of embodiment 70, wherein the antifolate agent is pemetrexed.

73. The method of embodiment 70, wherein the antifolate agent is raltitrexed.

74. The method of any one of embodiments 70-73, wherein the cancer is selected from bladder cancer, breast cancer, pancreatic adenocarcinoma, lung adenocarcinoma, lung squamous cell carcinoma, and head and neck adenocarcinoma,

75. The method of any one of embodiments 70-74, wherein the sample is a formalin-fixed, paraffin-embedded (FFPE) tissue sample, fresh or a frozen tissue sample, an exosome, or a bodily fluid obtained from the patient.

76. The method of embodiment 75, wherein the bodily fluid is blood or fractions thereof, urine, saliva, or sputum.

77. The method of any one of embodiments 70-76, wherein the measuring the expression levels of the plurality of classifier biomarkers is at a nucleic acid level by performing RNA sequencing, reverse transcriptase polymerase chain reaction (RT-PCR) or hybridization-based analyses.

78. The method of embodiment 77, wherein the RT-PCR is quantitative real time reverse transcriptase polymerase chain reaction (qRT-PCR).

79. The method of embodiment 78, wherein the RT-PCR is performed with primers specific to the classifier biomarkers selected from the plurality of classifier biomarkers of Table 1.

80. The method of any one of embodiments 70-79, further comprising comparing the detected levels of expression of the plurality of classifier biomarkers of Table 1 to the expression of the plurality of classifier biomarkers of Table 1 in at least one sample training set(s), wherein the at least one sample training set comprises expression data of the plurality of classifier biomarkers of Table 1 from a reference adenocarcinoma TRU (bronchioid) sample, expression data of the plurality of classifier biomarkers of Table 1 from a reference adenocarcinoma PP (magnoid) sample, expression data of the plurality of classifier biomarkers of Table 1 from a reference adenocarcinoma PI (squamoid) sample, or a combination thereof; and classifying the sample as TRU, PP, or PI based on the results of the comparing step.

81. The method of embodiment 80, wherein the comparing step comprises applying a statistical algorithm which comprises determining a correlation between the expression data obtained from the sample and the expression data from the at least one training set(s); and classifying the sample as a TRU, PP, or PI subtype based on the results of the statistical algorithm.

82. The method of embodiment 80 or 81, wherein the TRU subtype is indicative of the positive antifolate predictive response signature.

83. The method of any one of embodiments 70-82, wherein the plurality of classifier biomarkers comprises at least 8 biomarkers, at least 16 classifier biomarkers, at least 24 classifier biomarkers, at least 32 classifier biomarkers, at least 40 classifier biomarkers, or all 48 classifier biomarkers of Table 1.

84. The method of embodiment 70, wherein the method further comprises determining the expression level of one or more anti-folate drug targets in the sample obtained from the patient.

85. The method of embodiment 84, wherein the one or more anti-folate drug targets is selected from dhfr, gart, tyms, atic, or mthfd1l genes.

86. The method of any one of embodiments 70-85, wherein the method further comprises determining a tumor mutational burden of the sample obtained from the patient.

87. The method of any one of embodiments 70-86, wherein the method further comprises determining a proliferation signature of the sample obtained from the patient.

88. The method of embodiment 87, wherein the determining the proliferation signature in the sample obtained from the patient comprises measuring a nucleic acid expression level in the sample of at least five classifier genes from a plurality of classifier genes, wherein the plurality of classifier genes consists of only targeting protein for Xklp2 (TPX2), discs large homolog associated protein 5 (DLGAP5), Holliday junction recognition protein (HJURP), kinesin family member 4A (KIF4A), kinesin family member 2C (KIF2C), polo like kinase 1 (PLK1), maternal embryonic leucine zipper kinase (MELK), Cyclin B2 (CCNB2), budding uninhibited by benzimidazoles 1 (BUB1), kinesin family member 23 (KIF23), ubiquitin conjugating enzyme E2 C (UBE2C), kinesin family member 20A (KIF20A), trophinin associated protein (TROAP), aurora kinase B (AURKB), ribonucleotide reductase regulatory subunit M2 (RRM2), MYB proto-oncogene like 2 (MYBL2), antigen KI-67 (MK167), cell division cycle 20 (CDC20), centrosomal protein 55 (CEP55), topoisomerase 2-alpha (TOP2A), baculoviral IAP repeat containing 5 (BIRC5), abnormal spindle microtubule assembly (ASPM), extra spindle pole bodies like 1, separase (ESPL1), kinesin family member 18B (KIF18B), IQ motif containing GTPase activating protein 3 (IQGAP3), and effector cell protease receptor-1 (EPR1), wherein the expression level of nucleic acid of the at least five classifier genes represents a proliferation signature.

89. The method of embodiment 88, wherein the nucleic acid expression level is measured using an amplification, sequencing or hybridization assay.

90. The method of embodiment 89, wherein the amplification, hybridization and/or sequencing assay comprises performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays, Northern blotting, nCounter DX Analysis System or any other equivalent gene expression detection techniques.

91. The method of embodiment 90, wherein the nucleic acid expression level is detected by performing RNA-seq.

92. The method of any one of embodiments 88-91, wherein the measuring the nucleic acid expression level is for at least 10, 15, 20 or 25 classifier genes from the plurality of classifier genes.

93. The method of any one of embodiments 88-91, wherein the measuring the nucleic acid expression level is for all of the classifier genes from the plurality of classifier genes.

94. The method of embodiment 88, further comprising determining a proliferation score, wherein the determining the proliferation score comprises determining a mean nucleic acid expression level for the at least five classifier biomarkers from the plurality of classifier biomarkers.

95. The method of any one of embodiments 87-91, further comprising determining a level and/or activity of at least one additional marker involved in cell proliferation and mitosis.

96. The method of embodiment 95, wherein the at least one additional marker is Ki67 or CD31.

97. A method of detecting a proliferation signature in a sample obtained from a subject, the method comprising measuring a nucleic acid expression level of at least five classifier genes from a plurality of classifier genes in the sample, wherein the plurality of classifier genes consists of only targeting protein for Xklp2 (TPX2), discs large homolog associated protein 5 (DLGAP5), Holliday junction recognition protein (HJURP), kinesin family member 4A (KIF4A), kinesin family member 2C (KIF2C), polo like kinase 1 (PLK1), maternal embryonic leucine zipper kinase (MELK), Cyclin B2 (CCNB2), budding uninhibited by benzimidazoles 1 (BUB1), kinesin family member 23 (KIF23), ubiquitin conjugating enzyme E2 C (UBE2C), kinesin family member 20A (KIF20A), trophinin associated protein (TROAP), aurora kinase B (AURKB), ribonucleotide reductase regulatory subunit M2 (RRM2), MYB proto-oncogene like 2 (MYBL2), antigen KI-67 (MK167), cell division cycle 20 (CDC20), centrosomal protein 55 (CEP55), topoisomerase 2-alpha (TOP2A), baculoviral IAP repeat containing 5 (BIRC5), abnormal spindle microtubule assembly (ASPM), extra spindle pole bodies like 1, separase (ESPL1), kinesin family member 18B (KIF18B), IQ motif containing GTPase activating protein 3 (IQGAP3), and effector cell protease receptor-1 (EPR1), wherein the nucleic acid expression level of the at least five classifier genes represents a proliferation signature.

98. The method of embodiment 97, wherein the subject is suffering from or suspected of suffering from Cervical Kidney renal papillary cell carcinoma (KIRP), Breast Invasive Carcinoma (BRCA), Thyroid Cancer (THCA), Bladder Carcinoma (BLCA), Prostate Adenocarcinoma (PRAD), Kidney Chromophobe (KICH), Cervical Squamous Cell Carcinoma and Endocervical Adenocarcinoma (CESC), Kidney Renal Clear Cell Carcinoma (KIRC), Liver Hepatocellular Carcinoma (LIHC), Low Grade Glioma (LGG), Sarcoma (SARC), Lung Adenocarcinoma (LUAD), Colon Adenocarcinoma (COAD), Head-Neck Squamous Cell Carcinoma (HNSC), Uterine Corpus Endometrial Carcinoma (UCEC), Glioblastoma Multiforme (GBM), Esophageal Carcinoma (ESCA), Stomach Adenocarcinoma (STAD), Ovarian Cancer (OV), and Rectum Adenocarcinoma (READ).

99. The method of embodiment 97 or 98, wherein the sample is a formalin-fixed, paraffin-embedded (FFPE) tissue sample, fresh or a frozen tissue sample, an exosome, wash fluids, cell pellets, or a bodily fluid obtained from the subject.

100. The method of embodiment 99, wherein the sample is an FFPE tissue sample.

101. The method of embodiment 99, wherein the sample is a fresh frozen tissue sample.

102. The method of any one of embodiments 97-101, wherein the nucleic acid expression level is measured using an amplification, sequencing or hybridization assay.

103. The method of embodiment 102, wherein the amplification, hybridization and/or sequencing assay comprises performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays, Northern blotting, nCounter DX Analysis System or any other equivalent gene expression detection techniques.

104. The method of embodiment 103, wherein the nucleic acid expression level is detected by performing RNA-seq.

105. The method of any one of embodiments 97-104, wherein the measuring the nucleic acid expression level is for at least 10, 15, 20 or 25 classifier genes from the plurality of classifier genes.

106. The method of any one of embodiments 97-105, wherein the measuring the nucleic acid expression level is for all of the classifier genes from the plurality of classifier genes.

107. The method of any one of embodiments 97-106, further comprising determining a proliferation score, wherein the determining the proliferation score comprises determining a mean nucleic acid expression level for the at least five classifier biomarkers from the plurality of classifier biomarkers.

108. The method of any one of embodiments 97-107, further comprising determining a level and/or activity of at least one additional marker involved in cell proliferation and mitosis.

109. The method of embodiment 108, wherein the at least one additional marker is Ki67 or CD31.

110. A method of determining metastatic disease in a subject, the method comprising: measuring a nucleic acid expression level of at least five classifier genes from a plurality of classifier genes in a first sample obtained from the subject, wherein the plurality of classifier genes consists of only tpx2, dlgap5, hjurp, kif4a, kif2c, plk1, melk, ccnb2, bub1, kif23, ube2c, kif20a, troap, aurkb, rrm2, mybl2, mki67, cdc20, cep55, top2a, birc5, aspm, espl1, kif18b, iqgap3 and epr1, wherein the nucleic acid expression level of the at least five classifier genes represents a proliferation signature of the first sample; measuring the nucleic acid expression level of the same at least five classifier genes from the plurality of classifier genes in a second sample, wherein the nucleic acid expression level of the at least five classifier genes represents a proliferation signature of the second sample; and determining existence of a correlation between the proliferation signature of the first sample and the proliferation signature of the second sample, wherein the existence of a correlation is indicative of the likelihood of metastatic disease in the subject.

111. The method of embodiment 110, wherein the second sample is obtained from the subject, wherein the first and second samples are obtained from different regions of the subject's body.

112. The method of embodiment 110, wherein the second sample is obtained from a control subject that does not have metastatic disease, wherein the second sample is obtained from the same area of the body as the first sample.

113. The method of any one of embodiments 110-112, wherein the subject is suffering from or suspected of suffering from KIRP, BRCA, THCA, BLCA, PRAD, KICH, CESC, KIRC, LIHC, LGG, SARC, LUAD, COAD, HNSC, UCEC, GBM, ESCA STAD, OV and READ.

114. The method of any one of embodiments 110-112, wherein the first and/or second sample is a formalin-fixed, paraffin-embedded (FFPE) tissue sample, fresh or a frozen tissue sample, an exosome, wash fluids, cell pellets, or a bodily fluid obtained from the subject.

115. The method of embodiment 114, wherein the first sample and the second sample is an FFPE tissue sample.

116. The method of embodiment 114, wherein the first sample and the second sample is a fresh frozen tissue sample.

117. The method of any one of embodiments 110-116, wherein the nucleic acid expression level is measured using an amplification, sequencing or hybridization assay.

118. The method of embodiment 117, wherein the amplification, hybridization and/or sequencing assay comprises performing quantitative real time reverse transcriptase polymerase chain reaction (qRT-PCR), RNA-seq, microarrays, gene chips, nCounter Gene Expression Assay, Serial Analysis of Gene Expression (SAGE), Rapid Analysis of Gene Expression (RAGE), nuclease protection assays, Northern blotting, nCounter DX Analysis System or any other equivalent gene expression detection techniques.

119. The method of any one of embodiments 110-118, wherein the nucleic acid expression level is detected by performing RNA-seq.

120. The method of any one of embodiments 110-119, wherein the measuring the nucleic acid expression level is for at least 10, 15, 20 or 25 classifier genes from the plurality of classifier genes.

121. The method of any one of embodiments 110-119, wherein the measuring the nucleic acid expression level is for all of the classifier genes from the plurality of classifier genes.

122. The method of any one of embodiments 110-121, further comprising determining a level and/or activity of at least one additional marker involved in cell proliferation and mitosis.

123. The method of embodiment 122, wherein the at least one additional marker is Ki67 or CD31.

124. The method of any one of embodiments 110-123, wherein the determining the existence of a correlation comprises applying a statistical algorithm to the proliferation signature of the first sample and the proliferation signature of the second sample.

125. The method of any one of embodiments 110-123, wherein, prior to determining the existence of a correlation between the proliferation signature of the first sample and the proliferation signature of the second sample, determining a proliferation score for the first sample and the second sample, wherein the determining the proliferation score comprises determining a mean nucleic acid expression level across the at least five classifier biomarkers from the plurality of classifier biomarkers for the first sample and the second sample, whereby the determining the existence of a correlation entails determining the existence of a correlation between the proliferation score of the first sample and the proliferation score of the second sample.

126. The method of embodiment 125, wherein the determining the existence of a correlation comprises applying a statistical algorithm to the proliferation score of the first sample and the proliferation score of the second sample.

127. A method of treating a subject suffering from or suspected of suffering from cancer, the method comprising:
(a) determining a proliferation score of a sample obtained from the subject, wherein the determining the proliferation score comprises:
   (i) measuring a nucleic acid expression level of at least five classifier genes from a plurality of classifier genes in the sample obtained from the subject, wherein the plurality of classifier genes consists of only tpx2, dlgap5, hjurp, kif4a, kif2c, plk1, melk, ccnb2, bub1, kif23, ube2c, kif20a, troap, aurkb, rrm2, mybl2, mki67, cdc20, cep55, top2a, birc5, aspm, espl1, kif18b, iqgap3 and epr1; and
   (ii) calculating a mean nucleic acid expression level across the at least five classifier biomarkers from the plurality of classifier biomarkers, wherein the mean nucleic acid expression level across the at least five classifier biomarkers from the plurality of classifier biomarkers represents the proliferation score;
(b) determining a proliferation score of a control sample, wherein the determining the proliferation score comprises:
   (i) measuring a nucleic acid expression level of at least five classifier genes from a plurality of classifier genes in the control sample, wherein the plurality of classifier genes consists of only tpx2, dlgap5, hjurp, kif4a, kif2c, plk1, melk, ccnb2, bub1, kif23, ube2c, kif20a, troap, aurkb, rrm2, mybl2, mki67, cdc20, cep55, top2a, birc5, aspm, espl1, kif18b, iqgap3 and epr1; and
   (ii) calculating a mean nucleic acid expression level across the at least five classifier biomarkers from the plurality of classifier biomarkers, wherein the mean nucleic acid expression level across the at least five classifier biomarkers from the plurality of classifier biomarkers represents the proliferation score;
(c) comparing the proliferation score of the sample obtained from the subject to the proliferation score of the control sample; and
(d) administering a therapeutic agent to the subject based on the comparison between the proliferation score of the sample obtained from the subject and the control sample, thereby treating the cancer.

128. The method of embodiment 127, wherein the control sample is from a healthy subject.

129. The method of embodiment 127, wherein the control sample is a non-proliferative cancer sample.

130. The method of any one of embodiments 127-129, wherein the comparison shows an increased proliferation score of the sample obtained from the subject and the therapeutic agent administered is tailored to proliferative cancers.

131. The method of embodiment 130, wherein the therapeutic agent is selected from radiation therapy and anti-angiogenic therapeutic agents.

132. The method of any one of embodiments 127-131, wherein the cancer is selected from KIRP, BRCA, THCA, BLCA, PRAD, KICH, CESC, KIRC, LIHC, LGG, SARC, LUAD, COAD, HNSC, UCEC, GBM, ESCA, STAD, OV and READ.

133. The method of any one of embodiments 127-131, wherein the sample obtained from the subject and/or the control sample is a formalin-fixed, paraffin-embedded (FFPE) tissue sample, fresh or a frozen tissue sample, an exosome, wash fluids, cell pellets, or a bodily fluid obtained from the patient.

134. The method of embodiment 133, wherein the sample obtained from the subject and/or the control is an FFPE tissue sample.

135. The method of embodiment 133, wherein the sample obtained from the subject and/or the control is a fresh frozen tissue sample.

136. The method of any one of embodiments 127-135, wherein the nucleic acid expression level is measured using an amplification, sequencing or hybridization assay.

137. The method of embodiment 136, wherein the amplification, hybridization and/or sequencing assay comprises performing quantitative real time reverse transcriptase polymerase chain reaction (qRT-PCR), RNA-seq, microarrays, gene chips, nCounter Gene Expression Assay, Serial Analysis of Gene Expression (SAGE), Rapid Analysis of Gene Expression (RAGE), nuclease protection assays, Northern blotting, nCounter DX Analysis System or any other equivalent gene expression detection techniques.

138. The method of any one of embodiments 127-137, wherein the nucleic acid expression level is detected by performing RNA-seq.

139. The method of any one of embodiments 127-138, wherein the measuring the nucleic acid expression level is for at least 10, 15, 20 or 25 classifier genes from the plurality of classifier genes.

140. The method of any one of embodiments 127-138, wherein the measuring the nucleic acid expression level is for all of the classifier genes from the plurality of classifier genes.

141. The method of any one of embodiments 127-138, further comprising determining a level and/or activity of at least one additional marker involved in cell proliferation and mitosis.

142. The method of embodiment 141, wherein the at least one additional marker is Ki67 or CD31.

143. The method of any one of embodiments 127-142, further comprising determining a subtype of the sample obtained from the subject prior to administering the therapeutic agent and administering the therapeutic agent to the subject based on the comparison between the proliferation score of the sample obtained from the subject and the control sample and the subtype of the sample obtained from the subject.

144. The method of embodiment 143, wherein the determining the subtype is performed by histological examination of the sample.

145. The method of embodiment 143, wherein the determining the subtype is performed by gene expression analysis of the sample.

146. The method of embodiment 145, wherein the gene expression analysis of the sample is performed using a gene expression sub-typer that is publicly available.

147. A method of determining a disease outcome in a subject suffering from or suspected of suffering from cancer, the method comprising:
(a) determining a proliferation score of a sample obtained from the subject, wherein the determining the proliferation score comprises:
   (i) measuring a nucleic acid expression level of at least five classifier genes from a plurality of classifier genes in the sample obtained from the cancer patient, wherein the plurality of classifier genes consists of only tpx2, dlgap5, hjurp, kif4a, kif2c, plk1, melk, ccnb2, bub1, kif23, ube2c, kif20a, troap, aurkb, rrm2, mybl2, mki67, cdc20, cep55, top2a, birc5, aspm, espl1, kif18b, iqgap3 and epr1; and
   (ii) calculating a mean nucleic acid expression level across the at least five classifier biomarkers from the plurality of classifier biomarkers, wherein the mean nucleic acid expression level across the at least five classifier biomarkers from the plurality of classifier biomarkers represents the proliferation score;
(b) determining a proliferation score of a control sample, wherein the determining the proliferation score comprises:
   (i) measuring a nucleic acid expression level of at least five classifier genes from a plurality of classifier genes in the control sample, wherein the plurality of classifier genes consists of only tpx2, dlgap5, hjurp, kif4a, kif2c, plk1, melk, ccnb2, bub1, kif23, ube2c, kif20a, troap, aurkb, rrm2, mybl2, mki67, cdc20, cep55, top2a, birc5, aspm, espl1, kif18b, iqgap3 and epr1; and
   (ii) calculating a mean nucleic acid expression level across the at least five classifier biomarkers from the plurality of classifier biomarkers, wherein the mean nucleic acid expression level across the at least five classifier biomarkers from the plurality of classifier biomarkers represents the proliferation score; and
(c) comparing the proliferation score of the sample obtained from the subject to the proliferation score of the control sample, wherein an elevated proliferation score in the sample obtained from the subject is indicative of a poor disease outcome for the subject, wherein the subject suffers from or is suspected of suffering from a cancer selected from LUAD, LGG, LIHC, KIRC, KICH, MESO, ACC and KIRP.

148. The method of embodiment 147, wherein the disease outcome is expressed as overall patient survival.

149. The method of embodiment 147, wherein said disease outcome is expressed as recurrence-free survival.

150. The method of embodiment 147, wherein said disease outcome is expressed as distant recurrence-free survival.

151. The method of any one of embodiments 147-150, wherein the control sample is from a healthy subject.

152. The method of any one of embodiments 147-150, wherein the control sample is a non-proliferative cancer sample.

153. The method of any one of embodiments 147-150, wherein the sample obtained from the subject and/or the control sample is a formalin-fixed, paraffin-embedded (FFPE) tissue sample, fresh or a frozen tissue sample, an exosome, wash fluids, cell pellets, or a bodily fluid obtained from the patient.

154. The method of embodiment 153, wherein the sample obtained from the subject and/or the control is an FFPE tissue sample.

155. The method of embodiment 153, wherein the sample obtained from the subject and/or the control is a fresh frozen tissue sample.

156. The method of any one of embodiments 147-153, wherein the nucleic acid expression level is measured using an amplification, sequencing or hybridization assay.

157. The method of embodiment 156, wherein the amplification, hybridization and/or sequencing assay comprises performing quantitative real time reverse transcriptase polymerase chain reaction (qRT-PCR), RNA-seq, microarrays, gene chips, nCounter Gene Expression Assay, Serial Analysis of Gene Expression (SAGE), Rapid Analysis of Gene Expression (RAGE), nuclease protection assays, Northern blotting, nCounter DX Analysis System or any other equivalent gene expression detection techniques.

158. The method of any one of embodiments 147-157, wherein the nucleic acid expression level is detected by performing RNA-seq.

159. The method of any one of embodiments 147-158, wherein the measuring the nucleic acid expression level is for at least 10, 15, 20 or 25 classifier genes from the plurality of classifier genes.

160. The method of any one of embodiments 147-158, wherein the measuring the nucleic acid expression level is for all of the classifier genes from the plurality of classifier genes.

161. The method of any one of embodiments 147-160, further comprising determining a level and/or activity of at least one additional marker involved in cell proliferation and mitosis.

162. The method of embodiment 161, wherein the at least one additional marker is Ki67 or CD31.

163. The method of any one of embodiments 147-162, further comprising determining a subtype of the sample obtained from the subject.

164. The method of embodiment 163, wherein the determining the subtype is performed by histological examination of the sample.

165. The method of embodiment 163, wherein the determining the subtype is performed by gene expression analysis of the sample.

166. The method of embodiment 165, wherein the gene expression analysis of the sample is performed using a gene expression sub-typer that is publicly available.

167. A system for determining an antifolate predictive response signature of a sample obtained from a subject suffering from cancer, the system comprising:
(a) one or more processors; and
(b) one or more memories operatively coupled to at least one of the one or more processors and having instructions stored thereon that, when executed by at least one of the one or more processors, cause the system to
   (i) detect an expression level of each of a plurality of classifier biomarkers from Table 1;
   (ii) compare the expression levels of each of the plurality of classifier biomarkers from Table 1 to the expression levels of each of the plurality of classifier biomarkers from Table 1 in a control; and
   (iii) classifying the sample as TRU, PP, or PI based on the results of the comparing step.

168. The system of embodiment 167, wherein the control comprises at least one sample training set(s), wherein the at least one sample training set comprises expression data of the plurality of classifier biomarkers of Table 1 from a reference adenocarcinoma TRU (bronchioid) sample, expression data of the plurality of classifier biomarkers of Table 1 from a reference adenocarcinoma PP (magnoid) sample, expression data of the plurality of classifier biomarkers of Table 1 from a reference adenocarcinoma PI (squamoid) sample, or a combination thereof.

169. The system of embodiment 168, wherein the comparing step comprises applying a statistical algorithm which comprises determining a correlation between the expression data obtained from the sample and the expression data from the at least one training set(s); and classifying the sample as a TRU, PP, or PI subtype based on the results of the statistical algorithm.

170. The system of any one of embodiments 167-169, wherein the expression level of each of the plurality of classifier biomarkers from Table 1 is detected at the nucleic acid level.

171. The system of embodiment 170, wherein the nucleic acid level is RNA or cDNA.

172. The system of any one of embodiments 167-171, wherein the detecting the expression level comprises performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays, Northern blotting, or any other equivalent gene expression detection techniques.

173. The system of embodiment 172, wherein the expression level is detected by performing qRT-PCR.

174. The system of embodiment 172 or 173, wherein the detecting the expression level is performed using a device that is part of the system or in communication with at least one of the one or more processors, wherein upon receipt of instructions sent by the at least one of the one or more processors, perform the detection of the expression levels.

175. The system of any one of embodiments 167-174, wherein the plurality of classifier biomarkers from Table 1 comprises at least 8 classifier biomarkers, at least 16 classifier biomarkers, at least 24 classifier biomarkers, at least 32 classifier biomarkers, at least 40 classifier biomarkers or at least 48 classifier biomarkers from Table 1.

176. The system of any one of embodiments 167-175, wherein the plurality of classifier biomarkers of Table 1 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the classifier biomarkers from Table 1.

177. The system of any one of embodiments 167-176, wherein the plurality of classifier biomarkers of Table 1 comprise flgf, ctsh, sctr, cyp4b1, gpr116, adh1b, cbx7, hlf cep55, tpx2, bub1b, kif4a, ccnb2, kif14, melk, kif11 or any combination thereof.

178. The system of any one of embodiments 167-176, wherein the plurality of classifier biomarkers of Table 1 comprise fgl1, pbk, hspd1, tdg, prc1, dusp4, gtpbp4, zwint, tlr2, cd74, hla-dpb1, hla-dpa1, hla-dra, itgb2, fas, hla-drb1, plau, gbp1, dse, ccdc109b, tgfbi, cxcl10, lgals1, tubb6, gjb1, rap1gap, cacna2d2, selenbp1, tfcp2l1, sorbs2, unc13b, tacc2 or any combination thereof.

179. The system of any one of embodiments 167-178, wherein the plurality of classifier biomarkers of Table 1 comprises all the classifier biomarkers from Table 1.

180. The system of any one of embodiments 167-179, wherein the TRU subtype is indicative of a positive antifolate predictive response signature, wherein the positive antifolate predictive response signature selects the patient for treatment with an antifolate agent.

181. The system of embodiment 180, wherein the anti-folate agent is selected from pemetrexed, methotrexate, trimetrexate, lometrexol, raltitrexed and nolatrexed.

182. The system of embodiment 181, wherein the antifolate agent is pemetrexed.

183. The system of embodiment 180, wherein the antifolate agent is raltitrexed.

184. The system of any one of embodiments 167-183, wherein the cancer the patient is suffering from is selected from bladder cancer, breast cancer, pancreatic adenocarcinoma, lung adenocarcinoma, lung squamous cell carcinoma, and head and neck adenocarcinoma.

185. A system for determining a disease outcome in a subject suffering from or suspected of suffering from cancer, the system comprising:
(a) one or more processors; and
(b) one or more memories operatively coupled to at least one of the one or more processors and having instructions stored thereon that, when executed by at least one of the one or more processors, cause the system to
   (i) determine a proliferation score of a sample obtained from the subject, wherein the determining the proliferation score comprises:
      (a) measuring a nucleic acid expression level of at least five classifier genes from a plurality of classifier genes in the sample obtained from the cancer patient, wherein the plurality of classifier genes consists of only tpx2, dlgap5, hjurp, kif4a, kif2c, plk1, melk, ccnb2, bub1, kif23, ube2c, kif20a, troap, aurkb, rrm2, mybl2, mki67, cdc20, cep55, top2a, birc5, aspm, espl1, kif18b, iqgap3 and epr1; and
      (b) calculating a mean nucleic acid expression level across the at least five classifier biomarkers from the plurality of classifier biomarkers, wherein the mean nucleic acid expression level across the at least five classifier biomarkers from the plurality of classifier biomarkers represents the proliferation score;
   (ii) determine a proliferation score of a control sample, wherein the determining the proliferation score comprises:
      (a) measuring a nucleic acid expression level of at least five classifier genes from a plurality of classifier genes in the control sample, wherein the plurality of classifier genes consists of only tpx2, dlgap5, hjurp, kif4a, kif2c, plk1, melk, ccnb2, bub1, kif23, ube2c, kif20a, troap, aurkb, rrm2, mybl2, mki67, cdc20, cep55, top2a, birc5, aspm, espl1, kif18b, iqgap3 and epr1; and
      (b) calculating a mean nucleic acid expression level across the at least five classifier biomarkers from the plurality of classifier biomarkers, wherein the mean nucleic acid expression level across the at least five classifier biomarkers from the plurality of classifier biomarkers represents the proliferation score; and
   (iii) compare the proliferation score of the sample obtained from the subject to the proliferation score of the control sample, wherein an elevated proliferation score in the sample obtained from the subject is indicative of a poor disease outcome for the subject, wherein the subject suffers from or is suspected of suffering from a cancer.

186. The system of embodiment 185, wherein the cancer is selected from LUAD, LGG, LIHC, KIRC, KICH, MESO, ACC and KIRP.

187. The system of embodiment 185, wherein the disease outcome is expressed as overall patient survival.

188. The system of embodiment 185, wherein said disease outcome is expressed as recurrence-free survival.

189. The system of embodiment 185, wherein said disease outcome is expressed as distant recurrence-free survival.

190. The system of any one of embodiments 185-189, wherein the control sample is from a healthy subject.

191. The system of any one of embodiments 185-189, wherein the control sample is a non-proliferative cancer sample.

192. The system of any one of embodiments 185-191, wherein the sample obtained from the subject and/or the control sample is a formalin-fixed, paraffin-embedded (FFPE) tissue sample, fresh or a frozen tissue sample, an exosome, wash fluids, cell pellets, or a bodily fluid obtained from the patient.

193. The system of embodiment 192, wherein the sample obtained from the subject and/or the control is an FFPE tissue sample.

194. The system of embodiment 192, wherein the sample obtained from the subject and/or the control is a fresh frozen tissue sample.

195. The system of any one of embodiments 185-194, wherein the nucleic acid expression level is measured using an amplification, sequencing or hybridization assay.

196. The system of embodiment 195, wherein the amplification, hybridization and/or sequencing assay comprises performing quantitative real time reverse transcriptase polymerase chain reaction (qRT-PCR), RNA-seq, microarrays, gene chips, nCounter Gene Expression Assay, Serial Analysis of Gene Expression (SAGE), Rapid Analysis of Gene Expression (RAGE), nuclease protection assays, Northern blotting, nCounter DX Analysis System or any other equivalent gene expression detection techniques.

197. The system of embodiment 196, wherein the nucleic acid expression level is detected by performing RNA-seq.

198. The system of any one of embodiments 185-197, wherein the detecting the expression level is performed using a device that is part of the system or in communication with at least one of the one or more processors, wherein upon receipt of instructions sent by the at least one of the one or more processors, perform the detection of the expression levels.

199. The system of any one of embodiments 185-198, wherein the measuring the nucleic acid expression level is for at least 10, 15, 20 or 25 classifier genes from the plurality of classifier genes.

200. The system of embodiment 199, wherein the measuring the nucleic acid expression level is for all of the classifier genes from the plurality of classifier genes.

201. The system of embodiment 185-200, further comprising determining a level and/or activity of at least one additional marker involved in cell proliferation and mitosis.

202. The system of embodiment 201, wherein the at least one additional marker is Ki67 or CD31.

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary, to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

What is claimed is:

1. A method of detecting a biomarker in a sample obtained from a patient suffering from cancer, the method comprising measuring the nucleic acid expression level of a plurality of biomarkers selected from Table 1 using an amplification, hybridization and/or sequencing assay.

2. The method of claim 1, wherein the patient was previously diagnosed with a cancer selected from bladder cancer, breast cancer, pancreatic adenocarcinoma, lung adenocarcinoma, lung squamous cell carcinoma, and head and neck adenocarcinoma.

3. The method of claim 1, wherein the amplification, hybridization and/or sequencing assay comprises performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays, Northern blotting, or any other equivalent nucleic acid expression detection techniques.

4. The method of claim 3, wherein the nucleic acid expression level is detected by performing qRT-PCR.

5. The method of claim 4, wherein the detection of the nucleic acid expression level comprises using at least one pair of oligonucleotide primers per each of the plurality of biomarkers selected from Table 1.

6. The method of claim 1, wherein the sample is a formalin-fixed, paraffin-embedded (FFPE) tissue sample, fresh or a frozen tissue sample, an exosome, wash fluids, cell pellets, or a bodily fluid obtained from the patient.

7. The method of claim 6, wherein the bodily fluid is blood or fractions thereof, urine, saliva, or sputum.

8. The method of any one of the above claims, wherein the plurality of biomarkers comprises at least 8 biomarkers, at least 16 biomarkers, at least 24 biomarkers, at least 32 biomarkers, at least 40 biomarkers or at least 48 biomarkers of Table 1.

9. The method of any one of claims 1-7, wherein the plurality of biomarkers selected from Table 1 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 1.

10. The method of any one of claims 1-7, wherein the plurality of biomarkers selected from Table 1 comprise flgf, ctsh, sctr, cyp4b1, gpr116, adh1b, cbx7, hlf, cep55, tpx2, bub1b, kif4a, ccnb2, kif14, melk, kifl1 or any combination thereof.

11. The method of any one of claims 1-7, wherein the plurality of biomarkers of Table 1 comprise fgl1, pbk, hspd1, tdg, prc1, dusp4, gtpbp4, zwint, tlr2, cd74, hla-dpb1, hla-dpa1, hla-dra, itgb2, fas, hla-drb1, plau, gbp1, dse, ccdc109b, tgfbi, cxcl10, lgals1, tubb6, gjb1, rap1gap, cacna2d2, selenbp1, tfcp2l1, sorbs2, unc13b, tacc2 or any combination thereof.

12. The method of any one of claims 1-7, wherein the plurality of biomarkers comprises all the classifier biomarkers of Table 1.

13. A method of determining whether a patient suffering from cancer is likely to respond to treatment with an antifolate agent, the method comprising,
determining an antifolate predictive response signature of a sample obtained from a patient suffering from cancer; and
based on the antifolate predictive response signature, assessing whether the patient is likely to respond to treatment with an antifolate agent, wherein a positive antifolate predictive response signature predicts that the patient is likely to respond to the treatment with an antifolate agent.

14. A method for selecting a patient suffering from cancer for an antifolate agent, the method comprising, determining an antifolate predictive response signature of a sample obtained from a patient suffering from cancer; and selecting the patient for treatment with an antifolate agent if the antifolate response signature is positive.

15. The method of claim 13 or 14, wherein the anti-folate agent is selected from pemetrexed, methotrexate, trimetrexate, lometrexol, raltitrexed and nolatrexed.

16. The method of claim 15, wherein the antifolate agent is pemetrexed.

17. The method of claim 15, wherein the antifolate agent is raltitrexed.

18. The method of claim 13 or 14, wherein the cancer the patient is suffering from is selected from bladder cancer, breast cancer, pancreatic adenocarcinoma, lung adenocarcinoma, lung squamous cell carcinoma, and head and neck adenocarcinoma.

19. The method of claim 13 or 14, wherein the sample is a formalin-fixed, paraffin-embedded (FFPE) tissue sample, fresh or a frozen tissue sample, an exosome, or a bodily fluid obtained from the patient.

20. The method of claim 19, wherein the bodily fluid is blood or fractions thereof, urine, saliva, or sputum.

21. The method of claim 13 or 14, wherein the determining the antifolate predictive response signature of the sample obtained from the patient suffering from cancer comprises determining expression levels of a plurality of classifier biomarkers.

22. The method of claim 21, wherein the determining the expression levels of the plurality of classifier biomarkers is at a nucleic acid level by performing RNA sequencing, reverse transcriptase polymerase chain reaction (RT-PCR) or hybridization-based analyses.

23. The method of claim 21, wherein the plurality of classifier biomarkers for determining the antifolate predictive response signature is selected from Table 1.

24. The method of claim 23, wherein the RT-PCR is quantitative real time reverse transcriptase polymerase chain reaction (qRT-PCR).

25. The method of claim 24, wherein the RT-PCR is performed with primers specific to the classifier biomarkers selected from the plurality of classifier biomarkers of Table 1.

26. The method of claim 23, further comprising comparing the detected levels of expression of the plurality of classifier biomarkers of Table 1 to the expression of the plurality of classifier biomarkers of Table 1 in at least one sample training set(s), wherein the at least one sample training set comprises expression data of the plurality of classifier biomarkers of Table 1 from a reference adenocarcinoma TRU (bronchioid) sample, expression data of the plurality of classifier biomarkers of Table 1 from a reference adenocarcinoma PP (magnoid) sample, expression data of the plurality of classifier biomarkers of Table 1 from a reference adenocarcinoma PI (squamoid) sample, or a combination thereof; and classifying the sample as TRU, PP, or PI based on the results of the comparing step.

27. The method of claim 26, wherein the comparing step comprises applying a statistical algorithm which comprises determining a correlation between the expression data obtained from the sample and the expression data from the at least one training set(s); and classifying the sample as a TRU, PP, or PI subtype based on the results of the statistical algorithm.

28. The method of claim 26, wherein the TRU subtype is indicative of a positive antifolate predictive response signature, wherein the positive antifolate predictive response signature selects the patient for treatment with an antifolate agent.

29. The method of claim 23, wherein the plurality of classifier biomarkers comprises at least 8 biomarker nucleic acids, at least 16 biomarker nucleic acids, at least 24 biomarker nucleic acids, at least 32 biomarker nucleic acids, at least 140 biomarker nucleic acids or all 48 biomarker nucleic acids of Table 1.

30. The method of claim 23, wherein the plurality of classifier biomarkers selected from Table 1 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the classifier biomarkers from Table 1.

31. The method of claim 23, wherein the plurality of classifier biomarkers selected from Table 1 comprise figf, ctsh, sctr, cyp4b1, gpr116, adh1b, cbx7, hlf cep55, tpx2, bub1b, kif4a, ccnb2, kif14, melk, kifl1 or any combination thereof.

32. The method of claim 23, wherein the plurality of classifier biomarkers of Table 1 comprise fgl1, pbk, hspd1, tdg, prc1, dusp4, gtpbp4, zwint, tlr2, cd74, hla-dpb1, hla-dpa1, hla-dra, itgb2, fas, hla-drb1, plau, gbp1, dse, ccdc109b, tgfbi, cxcl10, lgals1, tubb6, gjb1, rap1gap, cacna2d2, selenbp1, tfcp2l1, sorbs2, unc13b, tacc2 or any combination thereof.

33. The method of claim 13 or 14, wherein the method further comprises determining the expression level of one or more anti-folate drug targets in the sample obtained from the patient.

34. The method of claim 33, wherein the one or more anti-folate drug targets is selected from dhfr, gart, tyms, atic, or mthfd1l genes.

35. The method of claim 13 or 14, wherein the method further comprises determining a tumor mutational burden of the tumor sample obtained from the patient.

36. The method of claim 13 or 14, wherein the method further comprises determining a proliferation signature of the tumor sample obtained from the patient.

37. The method of claim 36, wherein the determining the proliferation signature in the tumor sample obtained from a patient comprises measuring a nucleic acid expression level in the sample of at least five classifier genes from a plurality of classifier genes, wherein the plurality of classifier genes consists of only targeting protein for Xklp2 (TPX2), discs large homolog associated protein 5 (DLGAP5), Holliday junction recognition protein (HJURP), kinesin family member 4A (KIF4A), kinesin family member 2C (KIF2C), polo like kinase 1 (PLK1), maternal embryonic leucine zipper kinase (MELK), Cyclin B2 (CCNB2), budding uninhibited by benzimidazoles 1 (BUB1), kinesin family member 23 (KIF23), ubiquitin conjugating enzyme E2 C (UBE2C), kinesin family member 20A (KIF20A), trophinin associated protein (TROAP), aurora kinase B (AURKB), ribonucleotide reductase regulatory subunit M2 (RRM2), MYB proto-oncogene like 2 (MYBL2), antigen KI-67 (MK167), cell division cycle 20 (CDC20), centrosomal protein 55 (CEP55), topoisomerase 2-alpha (TOP2A), baculoviral IAP repeat containing 5 (BIRC5), abnormal spindle microtubule assembly (ASPM), extra spindle pole bodies like 1, separase (ESPL1), kinesin family member 18B (KIF18B), IQ motif containing GTPase activating protein 3 (IQGAP3), and effector cell protease receptor-1 (EPR1), wherein the nucleic acid expression level of the at least five classifier genes represents a proliferation signature.

38. The method of claim 37, wherein the nucleic acid expression level is measured using an amplification, sequencing or hybridization assay.

39. The method of claim 38, wherein the amplification, hybridization and/or sequencing assay comprises performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays, Northern blotting, nCounter DX Analysis System or any other equivalent gene expression detection techniques.

40. The method of claim 39, wherein the expression level is detected by performing RNA-seq.

41. The method of claim 37, wherein the measuring the nucleic acid expression level is for at least 10, 15, 20 or 25 classifier genes from the plurality of classifier genes.

42. The method of claim 37, wherein the measuring the nucleic acid expression level is for all of the classifier genes from the plurality of classifier genes.

43. The method of claim 37, further comprising determining a proliferation score, wherein the determining the proliferation score comprises determining a mean nucleic acid expression level for the at least five classifier biomarkers from the plurality of classifier biomarkers.

44. The method of claim 37, further comprising determining a level and/or activity of at least one additional marker involved in cell proliferation and mitosis.

45. The method of claim 44, wherein the at least one additional marker is Ki67 or CD31.

46. A method of treating cancer in a patient, the method comprising:
measuring the expression level of a plurality of classifier biomarkers in a sample obtained from a patient suffering from cancer, wherein the plurality of classifier biomarkers are selected from a set of classifier biomarkers listed in Table 1, wherein the measured expression levels of the plurality of classifier biomarkers provide an antifolate predictive response signature for the sample; and
administering an antifolate agent based on presence of a positive antifolate predictive response signature.

47. The method of claim 46, wherein the anti-folate agent is selected from pemetrexed, methotrexate, trimetrexate, lometrexol, raltitrexed and nolatrexed.

48. The method of claim 46, wherein the antifolate agent is pemetrexed.

49. The method of claim 46, wherein the antifolate agent is raltitrexed.

50. The method of claim 46, wherein the cancer is selected from bladder cancer, breast cancer, pancreatic adenocarcinoma, lung adenocarcinoma, lung squamous cell carcinoma, and head and neck adenocarcinoma,

51. The method of any one of claims 46-50, wherein the sample is a formalin-fixed, paraffin-embedded (FFPE) tissue sample, fresh or a frozen tissue sample, an exosome, or a bodily fluid obtained from the patient.

52. The method of claim 51, wherein the bodily fluid is blood or fractions thereof, urine, saliva, or sputum.

53. The method of claim 46, wherein the measuring the expression levels of the plurality of classifier biomarkers is at a nucleic acid level by performing RNA sequencing, reverse transcriptase polymerase chain reaction (RT-PCR) or hybridization-based analyses.

54. The method of claim 53, wherein the RT-PCR is quantitative real time reverse transcriptase polymerase chain reaction (qRT-PCR).

55. The method of claim 54, wherein the RT-PCR is performed with primers specific to the classifier biomarkers selected from the plurality of classifier biomarkers of Table 1.

56. The method of claim 46, further comprising comparing the detected levels of expression of the plurality of classifier biomarkers of Table 1 to the expression of the plurality of classifier biomarkers of Table 1 in at least one sample training set(s), wherein the at least one sample training set comprises expression data of the plurality of classifier biomarkers of Table 1 from a reference adenocarcinoma TRU (bronchioid) sample, expression data of the plurality of classifier biomarkers of Table 1 from a reference adenocarcinoma PP (magnoid) sample, expression data of the plurality of classifier biomarkers of Table 1 from a reference adenocarcinoma PI (squamoid) sample, or a combination thereof; and classifying the sample as TRU, PP, or PI based on the results of the comparing step.

57. The method of claim 56, wherein the comparing step comprises applying a statistical algorithm which comprises determining a correlation between the expression data obtained from the sample and the expression data from the at least one training set(s); and classifying the sample as a TRU, PP, or PI subtype based on the results of the statistical algorithm.

58. The method of claim 56 or 57, wherein the TRU subtype is indicative of the positive antifolate predictive response signature.

59. The method of claim 46, wherein the plurality of classifier biomarkers comprises at least 8 biomarkers, at least 16 classifier biomarkers, at least 24 classifier biomarkers, at least 32 classifier biomarkers, at least 40 classifier biomarkers, or all 48 classifier biomarkers of Table 1.

60. The method of claim 46, wherein the method further comprises determining the expression level of one or more anti-folate drug targets in the sample obtained from the patient.

61. The method of claim 60, wherein the one or more anti-folate drug targets is selected from dhfr, gart, tyms, atic, or mthfd1l genes.

62. The method of claim 46, wherein the method further comprises determining a tumor mutational burden of the sample obtained from the patient.

63. The method of claim 46, wherein the method further comprises determining a proliferation signature of the sample obtained from the patient.

64. The method of claim 63, wherein the determining the proliferation signature in the sample obtained from the patient comprises measuring a nucleic acid expression level in the sample of at least five classifier genes from a plurality of classifier genes, wherein the plurality of classifier genes consists of only targeting protein for Xklp2 (TPX2), discs large homolog associated protein 5 (DLGAP5), Holliday junction recognition protein (HJURP), kinesin family member 4A (KIF4A), kinesin family member 2C (KIF2C), polo like kinase 1 (PLK1), maternal embryonic leucine zipper kinase (MELK), Cyclin B2 (CCNB2), budding uninhibited by benzimidazoles 1 (BUB1), kinesin family member 23 (KIF23), ubiquitin conjugating enzyme E2 C (UBE2C), kinesin family member 20A (KIF20A), trophinin associated protein (TROAP), aurora kinase B (AURKB), ribonucleotide reductase regulatory subunit M2 (RRM2), MYB proto-oncogene like 2 (MYBL2), antigen KI-67 (MK167), cell division cycle 20 (CDC20), centrosomal protein 55 (CEP55), topoisomerase 2-alpha (TOP2A), baculoviral IAP repeat containing 5 (BIRC5), abnormal spindle microtubule assembly (ASPM), extra spindle pole bodies like 1, separase (ESPL1), kinesin family member 18B (KIF18B), IQ motif containing GTPase activating protein 3 (IQGAP3), and effector cell protease receptor-1 (EPR1), wherein the expression level of nucleic acid of the at least five classifier genes represents a proliferation signature.

65. The method of claim 64, wherein the nucleic acid expression level is measured using an amplification, sequencing or hybridization assay.

66. The method of claim 65, wherein the amplification, hybridization and/or sequencing assay comprises performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays, Northern blotting, nCounter DX Analysis System or any other equivalent gene expression detection techniques.

67. The method of claim 66, wherein the nucleic acid expression level is detected by performing RNA-seq.

68. The method of any one of claims 64-67, wherein the measuring the nucleic acid expression level is for at least 10, 15, 20 or 25 classifier genes from the plurality of classifier genes.

69. The method of any one of claims 64-67, wherein the measuring the nucleic acid expression level is for all of the classifier genes from the plurality of classifier genes.

70. The method of claim 64, further comprising determining a proliferation score, wherein the determining the proliferation score comprises determining a mean nucleic acid expression level for the at least five classifier biomarkers from the plurality of classifier biomarkers.

71. The method of any one of claims 63-67, further comprising determining a level and/or activity of at least one additional marker involved in cell proliferation and mitosis.

72. The method of claim 71, wherein the at least one additional marker is Ki67 or CD31.

73. A system for determining an antifolate predictive response signature of a sample obtained from a subject suffering from cancer, the system comprising:
(a) one or more processors; and
(b) one or more memories operatively coupled to at least one of the one or more processors and having instructions stored thereon that, when executed by at least one of the one or more processors, cause the system to
(i) detect an expression level of each of a plurality of classifier biomarkers from Table 1;
(ii) compare the expression levels of each of the plurality of classifier biomarkers from Table 1 to the expression levels of each of the plurality of classifier biomarkers from Table 1 in a control; and
(iii) classifying the sample as TRU, PP, or PI based on the results of the comparing step.

74. The system of claim 73, wherein the control comprises at least one sample training set(s), wherein the at least one sample training set comprises expression data of the plurality of classifier biomarkers of Table 1 from a reference adenocarcinoma TRU (bronchioid) sample, expression data of the plurality of classifier biomarkers of Table 1 from a reference adenocarcinoma PP (magnoid) sample, expression data of the plurality of classifier biomarkers of Table 1 from a reference adenocarcinoma PI (squamoid) sample, or a combination thereof.

75. The system of claim 74, wherein the comparing step comprises applying a statistical algorithm which comprises determining a correlation between the expression data obtained from the sample and the expression data from the at least one training set(s); and classifying the sample as a TRU, PP, or PI subtype based on the results of the statistical algorithm.

76. The system of claim 73, wherein the expression level of each of the plurality of classifier biomarkers from Table 1 is detected at the nucleic acid level.

77. The system of claim 76, wherein the nucleic acid level is RNA or cDNA.

78. The system of claim 73, wherein the detecting the expression level comprises performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays, Northern blotting, or any other equivalent gene expression detection techniques.

79. The system of claim 78, wherein the expression level is detected by performing qRT-PCR.

80. The system of claim 78, wherein the detecting the expression level is performed using a device that is part of the system or in communication with at least one of the one or more processors, wherein upon receipt of instructions sent by the at least one of the one or more processors, perform the detection of the expression levels.

81. The system of claim 73, wherein the plurality of classifier biomarkers from Table 1 comprises at least 8 classifier biomarkers, at least 16 classifier biomarkers, at least 24 classifier biomarkers, at least 32 classifier biomarkers, at least 40 classifier biomarkers or at least 48 classifier biomarkers from Table 1.

82. The system of claim 73, wherein the plurality of classifier biomarkers of Table 1 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the classifier biomarkers from Table 1.

83. The system of claim 73, wherein the plurality of classifier biomarkers of Table 1 comprise flgf, ctsh, sctr, cyp4b1, gpr116, adh1b, cbx7, hlf cep55, tpx2, bub1b, kif4a, ccnb2, kif14, melk, kif11 or any combination thereof.

84. The system of claim 73, wherein the plurality of classifier biomarkers of Table 1 comprise fgl1, pbk, hspd1, tdg, prc1, dusp4, gtpbp4, zwint, tlr2, cd74, hla-dpb1, hla-dpa1, hla-dra, itgb2, fas, hla-drb1, plau, gbp1, dse, ccdc109b, tgfbi, cxcl10, lgals1, tubb6, gjb1, rap1gap, cacna2d2, selenbp1, tfcp2l1, sorbs2, unc13b, tacc2 or any combination thereof.

85. The system of claim 73, wherein the plurality of classifier biomarkers of Table 1 comprises all the classifier biomarkers from Table 1.

86. The system of claim 73, wherein the TRU subtype is indicative of a positive antifolate predictive response signature, wherein the positive antifolate predictive response signature selects the patient for treatment with an antifolate agent.

87. The system of claim 86, wherein the anti-folate agent is selected from pemetrexed, methotrexate, trimetrexate, lometrexol, raltitrexed and nolatrexed.

88. The system of claim 87, wherein the antifolate agent is pemetrexed.

89. The system of claim 86, wherein the antifolate agent is raltitrexed.

90. The system of claim 73, wherein the cancer the patient is suffering from is selected from bladder cancer, breast cancer, pancreatic adenocarcinoma, lung adenocarcinoma, lung squamous cell carcinoma, and head and neck adenocarcinoma.


SEQUENCE LISTING (74 sequences)

SEQ ID NO: 1 - DNA, 2122 bp, Homo sapiens
ctctagcttctagcacaatgtctgtcatttaatgggcattcagtacataatagataggtaattgaatgaatggatttagatgcctgggtgttttctttctacttgtattctattccccacatcctctccaaagatttcagcatcatcaagacatattatcctgtgtcttccttctgaagatttaaattatagtgatcaccaaatccatgttgatatgataacaatggttaagaagtttgaatatgttattttctctaagcaatacagttgtatattaatcagaatctcccagagaaagccccttagagcaggcagttccataaaaggaatgtcaggaacttctgcaggctggatgaggacatacactgagtgaacaagtctgtcaactgctctaaagacactgtcaatgccatataaaagctggccttgttcatctagcagatcatgggggtgagacagtccagtgcctctctgcccctacaatatatgaagaaaggggaagaaacacacattgcctcctggtagattaggggaggacttcttgtacatttttagcaaaatccgagttcagacccagcaattagacaaaagggtcagcaaactatggtcgatgggctacatctgcccactgcatgtttttgtaaatgaagttttattgggacacagccatacctattcacttttgcattatctatggctgcttttgcactttgatgaaagagttgagtagttgagacagacactgtttgacccacaaaatctaaaatacatactatctagcccttcctagcaaacatttgccaacccctagaaaacttttaaaaaatgatgctcttcgacttgaaatttttataatcttctgtttattgtcaataggtgctgctgattttcaaaaaatgacttagaatcctcaaataagtatacagccttatatgctgcttagacaccaaagctatgataagatttgtcagttctttgtatcagatgggttcaattcttgacatcagctaatgttataaaaataactacaaaattggttgtaatagaaccaaatctaagactctttttaataaaaaagatcaccattgctggtgagaaacctatactgaatgggtgagatgccattgcatttatctgaagtttcgtctttataatcaataatcatatacaatcctagtcaagaaactcatcttacagtccttggggattgaacactgtgaaaataaacttttactcttcggatgtgcttggattttgttttaaattaagaactacagaaatatatatcatttctagtggtagaggaaaatattgccacccagaactttctgaatgtctctttctgccctaggtccatttgtgatgaacaccaatgaagagatttctcaagctattcttgatttcagaaacgcaaaaaatgggtttgaaagggccaaaacctggaaatcaaagattgggaactagtggaaagcggaagagcaggtcttgatgtgtcctagaattttgccatttctgagattgagccattgaaggcattccatttctaaagcttatttagccggtgcttctaaagaattccacactaacgtgataacatggtttttgtaacaataaatgtaggatatttcctggcacatgcaaataaacctaatcattgtttctttaaaaatcagtgtttttcatttgagatacctaagttactaagcttctgtttaaaaagtcgttttgtgtatgtttgcctttttttgcatgtatggatggatgtttttattatttttgtgcatgtgcaggttttaaaaatcatatttgattgctttctgtgtgtcattggcagcagatgcatgagcatctgaggtcccttcttaagcaatcccactgagatacaaaggtcaaagctatgtgctggggtttcatgttacaggttatctgctttaaaataacggcagccctgaacatttgagtcagttcttaaaactgccctgctattggtagggacgcaacaggattacagccaagacttctctgcattttctgccaaaatctgtgtcagatttaagacacatgcttctgcaagcttccatgaaggttgtgcaaaaaagtttcaatccagagttgggttcca

SEQ ID NO: 2 - DNA, 1514 bp, Homo sapiens
ttcgcgctcccgccgctcctccacgctcgtgccgctccccccccgcgctcccagttgacgctctgggccgccacctccgcggaccctgagcgcaagagccaagccgccagcgctgcgatgtgggccacgctgccgctgctctgcgccggggcctggctcctgggagtccccgtctgcggtgccgccgaactgtgcgtgaactccttagagaagtttcacttcaagtcatggatgtctaagcaccgtaagacctacagtacggaggagtaccaccacaggctgcagacgtttgccagcaactggaggaagataaacgcccacaacaatgggaaccacacatttaaaatggcactgaaccaattttcagacatgagctttgctgaaataaaacacaagtatctctggtcagagcctcagaattgctcagccaccaaaagtaactaccttcgaggtactggtccctacccaccttccgtggactggcggaaaaaaggaaattttgtctcacctgtgaaaaatcagggtgcctgcggcagttgctggactttctccaccactggggccctggagtctgcgatcgccatcgcaaccggaaagatgctgtccttggcggaacagcagctggtggactgcgcccaggacttcaataatcacggctgccaagggggtctccccagccaggctttcgagtatatcctgtacaacaaggggatcatgggtgaagacacctacccctaccagggcaaggatggttattgcaagttccaacctggaaaggccatcggctttgtcaaggatgtagccaacatcacaatctatgacgaggaagcgatggtggaggctgtggccctctacaaccctgtgagctttgcctttgaggtgactcaggacttcatgatgtatagaacgggcatctactccagtacttcctgccataaaactccagataaagtaaaccatgcagtactggctgttgggtatggagaaaaaaatgggatcccttactggatcgtgaaaaactcttggggtccccagtggggaatgaacgggtacttcctcatcgagcgcggaaagaacatgtgtggcctggctgcctgcgcctcctaccccatccctctggtgtgagccgtggcagccgcagcgcagactggcggagaaggagaggaacgggcagcctgggcctgggtggaaatcctgccctggaggaagttgtggggagatccactgggacccccaacattctgccctcacctctgtgcccagcctggaaacctacagacaaggaggagttccaccatgagctcacccgtgtctatgacgcaaagatcaccagccatgtgccttagtgtccttcttaacagactcaaaccacatggaccacgaatattctttctgtccagaagggctactttccacatatagagctccagggactgtcttttctgtattcgctgttcaataaacattgagtgagcacctccccagatggagcatgctggtcctggaaaaaaaaa

SEQ ID NO: 3 - DNA, 1833 bp, Homo sapiens
aaagccgcgtcctgatcaatggggcgggcggcttagcgcgcgcggccacctggtccgaggaggagcagtcccggggcccgccgcaggtcgggtggctcagccatggctcctcggggcgcagcggccggccggagcccgggaccctgcgcggggcgctgagctcccgagcgggcagagggcacgggcaggcggacgtcggggcgccctcggggaacgtgcgggcaccatgcgtccccacctgtcgccgccgctgcagcagctactactgccggtgctgctcgcctgcgccgcgcactcgactggagcccttccccgactatgtgacgtgctacaagtgctgtgggaagagcaagaccagtgcctgcaggaactctccagagagcagacaggagacctgggcacggagcagccagtgccaggttgtgaggggatgtgggacaacataagctgctggccctcttctgtgccgggccggatggtggaggtggaatgcccgagattcctccggatgctcaccagcagaaatggttccttgttccgaaactgcacacaggatggctggtcagaaaccttccccaggcctaatctggcctgtggcgttaatgtgaacgactcttccaacgagaagcggcactcctacctgctgaagctgaaagtcatgtacaccgtgggctacagctcctccctggtcatgctcctggtcgcccttggcatcctctgtgctttccggaggctccactgcactcgcaactacatccacatgcacctgttcgtgtccttcatccttcgtgccctgtccaacttcatcaaggacgccgtgctcttctcctcagatgatgtcacctactgcgatgcccacagggcgggctgcaagctggtcatggtgctgttccagtactgcatcatggccaactactcctggctgctggtggaaggcctctaccttcacacactcctcgccatctccttcttctctgaaagaaagtacctccagggatttgtggcattcggatggggttctccagccatttttgttgctttgtgggctattgccagacactttctggaagatgttgggtgctgggacatcaatgccaacgcatccatctggtggatcattcgtggtcctgtgatcctctccatcctgattaatttcatccttttcataaacattctaagaatcctgatgagaaaacttagaacccaagaaacaagaggaaatgaagtcagccattataagcgcctggccaggtccactctcctgctgatccccctctttggcatccactacatcgtcttcgccttctccccagaggacgctatggagatccagctgttttttgaactagcccttggctcattccagggactggtggtggccgtcctctactgcttcctcaatggggaggtgcagctggaggttcagaagaagtggcagcaatggcacctccgtgagttcccactgcaccccgtggcctccttcagcaacagcaccaaggccagccacttggagcagagccagggcacctgcaggaccagcatcatctgagaggctggagcagggtcacccacggacagagaccaagagaggtcctgcgaaggctgggcactgctgtgggacagccagtcttcccagcagacaccctgtgtcctccttcagctgaagatgcccctccccaggccttggactcttccgaagggatgtgaggcactgtggggcaggacaagggcctgggatttggttcgtttgctcttctgggaagagaagttcaggggtcccagaaagggacagggaaataaatggtgcctgggatgagattcaaaaaaaaaaaa

SEQ ID NO: 4 - DNA, 2128 bp, Homo sapiens
aagatgcatgggtccagagtataaaggaacccaggagcagctgaaggcaggtcagatgaaggctaggtggctggaactgcaaccatggtgcccagcttcctctccctgagcttctcctccttgggcctgtgggcttctgggctgatcttggtcttaggctttctcaagctcatccacctgctgctgcggaggcagacgttggctaaggctatggacaaattcccagggcctcccacccactggctttttggacatgccctcgagatccaggagacggggagcctggacaaagtggtgtcctgggcccaccagttcccgtatgcccacccactctggttcggacagttcattggcttcctgaacatctatgagcctgactatgccaaagctgtgtacagccgtggggggagaggcctgctggttcttgaggggcccaagtggttgcagcaccgcaagctgctcacacctggctttcattatgatgtgctgaagccctatgtggccgtgttcactgagtctacacgtatcatgctggacaagtgggaagagaaagctcgggagggtaagtcctttgacatcttctgcgatgtgggtcacatggcgctgaacacactcatgaagtgcacctttggaagaggagacaccggcctgggccacagcagggacagcagctactaccttgcagtcagcgatctcactctgttgatgcagcagcgccttgtgtccttccagtaccataatgacttcatctactggctcaccccacatggccgccgcttcctgcgggcctgccaggtggcccatgaccatacagaccaggtcatcagggagcggaaggcagccctgcaggatgagaaggtgcggaagaagatccagaaccggaggcacctggacttcctggacattctcctgggtgcccgggatgaagatgacatcaaactgtcagatgcagacctccgggctgaagtggacacattcatgtttgaaggccatgacaccaccaccagtggtatctcctggtttctctactgcatggccctgtaccctgagcaccagcatcgttgtagagaggaggtccgcgagatcctaggggaccaggacttcttccagtgggatgatctgggcaaaatgacttatctgaccatgtgcatcaaggagagcttccgcctctacccacctgtgccccaggtgtaccgccagctcagcaagcctgtcacctttgtggatggccggtctctacctgcaggaagcctgatctctatgcatatctatgccctccataggaacagtgctgtatggcccgaccctgaggtctttgactctctgcgcttttccactgagaatgcatccaaacgccatccctttgcctttatgcccttctctgctgggcccaggaactgcattgggcagcagtttgccatgagtgagatgaaggtggtcacagccatgtgcttgctccgctttgagttctctctggacccctcacggctgcccatcaagatgccccagcttgtcctgcgctccaagaatggctttcacctccacctgaagccactgggccctgggtctgggaagtagctctgatgagaatggggtcccagatggctcaggctgtgacctccctgggcaccaccctccccaggctgggtgtggaggagttggggccccctgccttcaggaggcttgtagtttagaagggaagtaggcattaccatagacgactcctagaggacagtgctatgtaaaaatgtgtgtctataaatgtttatcatgcatgtattctagagctcattcatttattcaacaaacatttggtgagcacctatttcgttcgagaaacttcatttatctcctataattggcaaacttaaaaatgcagcagaaacttacattccaaccttagagactcatagtgagcacaaggaaagttttgccctgagattcatggttatggctgggtaccaccaaatagaagaatggcttaggggagtgccccttcactgagatgtgtttctttgttgaactttgtgtgtgtgtgtttagaatataacagacataagaaaaaattacctaaatgaagactgtacaaaataataaataattctgaagcagaaaaaaaaaaaaaaaa

SEQ ID NO: 5 - DNA, 3903 bp, Homo sapiens
atgaaatccccaaggagaaccactttgtgcctcatgtttattgtgatttattcttccaaagctgcactgaactggaattacgagtctactattcatcctttgagtcttcatgaacatgaaccagctggtgaagaggcactgaggcaaaaacgagcagttgccacaaaaagtcctacggctgaagaatacactgttaatattgagatcagttttgaaaatgcatccttcctggatcctatcaaagcctacttgaacagcctcagttttccaattcatgggaataacactgaccaaattaccgacattttgagcataaatgtgacaacagtctgcagacctgctggaaatgaaatctggtgctcctgcgagacaggttatgggtggcctcgggaaaggtgtcttcacaatctcatttgtcaagagcgtgacgtcttcctcccagggcaccattgcagttgccttaaagaactgcctcccaatggacctttttgcctgcttcaggaagatgttaccctgaacatgagagtcagactaaatgtaggctttcaagaagacctcatgaacacttcctccgccctctataggtcctacaagaccgacttggaaacagcgttccggaagggttacggaattttaccaggcttcaagggcgtgactgtgacagggttcaagtctggaagtgtggttgtgacatatgaagtcaagactacaccaccatcacttgagttaatacataaagccaatgaacaagttgtacagagcctcaatcagacctacaaaatggactacaactcctttcaagcagttactatcgaaagcaatttctttgtcacaccagaaatcatctttgaaggggacacagtcagtctggtgtgtgaaaaggaagttttgtcctccaatgtgtcttggcgctatgaagaacagcagttggaaatccagaacagcagcagattctcgatttacaccgcacttttcaacaacatgacttcggtgtccaagctcaccatccacaacatcactccaggtgatgcaggtgaatatgtttgcaaactgatattagacatttttgaatatgagtgcaagaagaaaatagatgttatgcccatccaaattttggcaaatgaagaaatgaaggtgatgtgcgacaacaatcctgtatctttgaactgctgcagtcagggtaatgttaattggagcaaagtagaatggaagcaggaaggaaaaataaatattccaggaacccctgagacagacatagattctagctgcagcagatacaccctcaaggctgatggaacccagtgcccaagcgggtcgtctggaacaacagtcatctacacttgtgagttcatcagtgcctatggagccagaggcagtgcaaacataaaagtgacattcatctctgtggccaatctaacaataaccccggacccaatttctgtttctgagggacaaaacttttctataaaatgcatcagtgatgtgagtaactatgatgaggtttattggaacacttctgctggaattaaaatataccaaagattttataccacgaggaggtatcttgatggagcagaatcagtactgacagtcaagacctcgaccagggagtggaatggaacctatcactgcatatttagatataagaattcatacagtattgcaaccaaagacgtcattgttcacccgctgcctctaaagctgaacatcatggttgatcctttggaagctactgtttcatgcagtggttcccatcacatcaagtgctgcatagaggaggatggagactacaaagttactttccatacgggttcctcatcccttcctgctgcaaaagaagttaacaaaaaacaagtgtgctacaaacacaatttcaatgcaagctcagtttcctggtgttcaaaaactgttgatgtgtgttgtcactttaccaatgctgctaataattcagtctggagcccatctatgaagctgaatctggttcctggggaaaacatcacatgccaggatcccgtaataggtgtcggagagccggggaaagtcatccagaagctatgccggttctcaaacgttcccagcagccctgagagtcccattggcgggaccatcacttacaaatgtgtaggctcccagtgggaggagaagagaaatgactgcatctctgccccaataaacagtctgctccagatggctaaggctttgatcaagagcccctctcaggatgagatgctccctacatacctgaaggatctttctattagcatagacaaagcggaacatgaaatcagctcttctcctgggagtctgggagccattattaacatccttgatctgctctcaacagttccaacccaagtaaattcagaaatgatgacgcacgtgctctctacggttaatgtcatccttggcaagcccgtcttgaacacctggaaggttttacaacagcaatggaccaatcagagttcacagctactacattcagtggaaagattttcccaagcattacagtcgggagatagccctcctttgtccttctcccaaactaatgtgcagatgagcagcatggtaatcaagtccagccacccagaaacctatcaacagaggtttgttttcccatactttgacctctggggcaatgtggtcattgacaagagctatctagaaaacttgcagtcggattcgtctattgtcaccatggctttcccaactctccaagccatccttgcccaggatatccaggaaaataactttgcagagagcttagtgatgacaaccactgtcagccacaatacaactatgccattcaggatttcaatgacttttaagaacaatagcccttcaggcggcgaaacgaagtgtgtcttctggaacttcaggcttgccaacaacacaggggggtgggacagcagtgggtgctatgtagaagaaggtgatggggacaatgtcacctgtatctgtgaccacctaacatcattctccatcctcatgtcccctgactccccagatcctagttctctcctgggaatactcctggatattatttcttatgttggggtgggcttttccatcttgagcttggcagcctgtctagttgtggaagctgtggtgtggaaatcggtgaccaagaaccggacttcttatatgcgccacacctgcatagtgaatatcgctgcctcccttctggtcgccaacacctggttcattgtggtcgctgccatccaggacaatcgctacatactctgcaagacagcctgtgtggctgccaccttcttcatccacttcttctacctcagcgtcttcttctggatgctgacactgggcctcatgctgttctatcgcctggttttcattctgcatgaaacaagcaggtccactcagaaagccattgccttctgtcttggctatggctgcccacttgccatctcggtcatcacgctgggagccacccagccccgggaagtctatacgaggaagaatgtctgttggctcaactgggaggacaccaaggccctgctggctttcgccatcccagcactgatcattgtggtggtgaacataaccatcactattgtggtcatcaccaagatcctgaggccttccattggagacaagccatgcaagcaggagaagagcagcctgtttcagatcagcaagagcattggggtcctcacaccactcttgggcctcacttggggttttggtctcaccactgtgttcccagggaccaaccttgtgttccatatcatatttgccatcctcaatgtcttccagggattattcattttactctttggatgcctctgggatctgaagtcaacatccctgggttcatccacacctgtgttttctatgagttctccaatatcaaggagatttaacaatttgtttggtaaaacaggttaa

SEQ ID NO: 6 - DNA, 2829 bp, Homo sapiens
acaagcaaacaaaataaatatctgtgcaatatatctgctttatgcactcaagcagagaagaaatccacaaagactcacagtctgctggtgggcagagaagacagaaacgacatgagcacagcaggaaaagaaagttgtctgacagaagtttgatgagaggaatttgactcagaagactgaaatatcctttaaccttactataaaatatcttacaaaatactcattgatttcacagcattagaatcatcaaggtaatcaaatgcaaagcagctgtgctatgggaggtaaagaaacccttttccattgaggatgtggaggttgcacctcctaaggcttatgaagttcgcattaagatggtggctgtaggaatctgtcacacagatgaccacgtggttagtggcaacctggtgaccccccttcctgtgattttaggccatgaggcagccggcatcgtggagagtgttggagaaggggtgactacagtcaaaccaggtgataaagtcatcccgctctttactcctcagtgtggaaaatgcagagtttgtaaaaacccggagagcaactactgcttgaaaaatgatctaggcaatcctcgggggaccctgcaggatggcaccaggaggttcacctgcagggggaagcccattcaccacttccttggcaccagcaccttctcccagtacacggtggtggatgagaatgcagtggccaaaattgatgcagcctcgcccctggagaaagtctgcctcattggctgtggattctcgactggttatgggtctgcagttaacgttgccaaggtcaccccaggctctacctgtgctgtgtttggcctgggaggggtcggcctatctgctgttatgggctgtaaagcagctggagcagccagaatcattgcggtggacatcaacaaggacaaatttgcaaaggccaaagagttgggtgccactgaatgcatcaaccctcaagactacaagaaacccattcaggaagtgctaaaggaaatgactgatggaggtgtggatttttcgtttgaagtcatcggtcggcttgacaccatgatggcttccctgttatgttgtcatgaggcatgtggcacaagcgtcatcgtaggggtacctcctgcttcccagaacctctcaataaaccctatgctgctactgactggacgcacctggaagggggctgtttatggtggctttaagagtaaagaaggtatcccaaaacttgtggctgattttatggctaagaagttttcactggatgcgttaataacccatgttttaccttttgaaaaaataaatgaaggatttgacctgcttcactctgggaaaagtatccgtaccgtcctgacgttttgaggcaatagagatgccttcccctgtagcagtcttcagcctcctctaccctacaagatctggagcaacagctaggaaatatcattaattcagctcttcagagatgttatcaataaattacacatgggggctttccaaagaaatggaaattgatgggaaattatttttcaggaaaatttaaaattcaagtgagaagtaaataaagtgttgaacatcagctggggaattgaagccaacaaaccttccttcttaaccattctactgtgtcacctttgccattgaggaaaaatattcctgtgacttcttgcatttttggtatcttcataatctttagtcatcgaatcccagtggaggggacccttttacttgccctgaacatacacatgctgggccattgtgattgaagtcttctaactctgtctcagttttcactgtcgacattttcctttttctaataaaaatgtaccaaatccctggggtaaaagctagggtaaggtaaaggatagactcacatttacaagtagtgaaggtccaagagttctaaatacaggaaatttcttaggaactcaaataaaatgccccacattttactacagtaaatggcagtgtttttatgacttttatactatttctttatggtcgatatacaattgattttttaaaataatagcagatttcttgcttcatatgacaaagcctcaattactaattgtaaaaactgaactattcccagaatcatgttcaaaaaatctgtaatttttgctgatgaaagtgcttcattgactaaacagtattagtttgtggctataaatgattatttagatgatgactgaaaatgtgtataaagtaattaaaagtaatatggtggctttaagtgtagagatgggatggcaaatgctgtgaatgcagaatgtaaaattggtaactaagaaatggcacaaacaccttaagcaatatattttcctagtagatatatatatacacatacatatatacacatatacaaatgtatatttttgcaaaattgttttcaatctagaacttttctattaactaccatgtcttaaaatcaagtctataatcctagcattagtttaatattttgaatatgtaaagacctgtgttaatgctttgttaatgcttttcccactctcatttgttaatgctttcccactctcaggggaaggatttgcattttgagctttatctctaaatgtgacatgcaaagattattcctggtaaaggaggtagctgtctccaaaaatgctattgttgcaatatctacattctatttcatattatgaaagaccttagacataaagtaaaatagtttatcatttactgtgtgatcttcagtaagtctctcaggctctctgagcttgttcatcctttgttttgaaaaaattactcaaccaatccattacagcttaaccaagattaaatgggatgatgttaaaaaaaaaaaaaaaaa

SEQ ID NO: 7 - DNA, 4081 bp, Homo sapiens
ccgaggaaaacgttgcgcaggttcaaaaatggaacgtcggcggcgtgagggagcgcgagggggtgtgcgcgcgtgcgcgtgcgcgtgcgcgcccggacgagggtgacggggaccccgccagccccagcatcgcgcgccgcagccgcggccccgcagctccgcccccggcccggcccggccccgggcccgctcgcccgccgccccgcatggagctgtcagccatcggcgagcaggtgttcgccgtggagagcatccggaagaagcgcgtgcggaagggtaaagtcgagtatctggtgaagtggaaaggatggcccccaaagtacagcacgtgggagccagaagagcacatcttggacccccgcctcgtcatggcctacgaggagaaggaggagagagaccgagcatcggggtataggaagagaggtccgaaacccaagcggcttctgctgcagcggctgtacagcatggacctgcggagctcccacaaggccaagggcaaggagaagctctgcttctccctgacgtgcccactcggcagcgggagccctgagggggtggtcaaggcgggggcacctgagctggtggacaagggccccttggtgcccaccctgcccttcccgctccgcaagccccgaaaggcccacaagtacctgcggctctcgcgcaagaagttcccgccccgcgggcccaacctggagagccacagccatcgacgggagctcttcctgcaggagccaccggccccagacgtcctgcaggcggctggcgagtgggagcctgctgcgcagccccctgaagaggaggcagatgccgacctggccgaggggccccctccctggacacctgcgctcccctcaagtgaggtgaccgtgaccgacatcaccgccaactccatcaccgtcaccttccgcgaggcccaggcagctgagggcttcttccgagaccgcagtgggaagttctgaatcaccgtttttactcttcttaaactgttttcttttgggcttggggtgggacttccagagatagggatgggttgggggcggggtaattattttatttaaaaaaataccgagcagcaaaaggggagaagatcccactactctcccaccacctgccctttctctgagggacgtttaccacgaggcctcaggctggggatggagagagttgctctgggagttggggtaccacccccagggcaggatggggacaggatcacctgcccgggacaccaccattatcattctcctctagtgacgcagcagctggttctgggagttaaaggagcattggaaggcccaaaccctctcccttgagtggccaccccagcctggttggctggttttccccttttctcttgtttcaattgggtctttaccttgaactctcctctctggctttgcggtgggctgtggaggctggttttgaccaaaagtgagtggggcgggaggaaggggcaggaggaagggttgaggttacttggggcgagtcccttccccttcagagaggcttctatccttcccagggaggaggcgccgctgagacccttctgctgagagctctgccctcccctcatcacctggcctgtgcagaaacgctcatgcacacctggctgcacaggtgtgcacgcattacccttcgcgtgtacgttcccatgtgccccgtgaaagcatgtgtggctgcagacgtgtccacatgggccttgcgaacctgggttagaaaccctggccaggcgaacgtggggtgattcacagcacaaaagacctcaccaccacacctgcactcaccccaccttgcatgcaccttgctacctgcttgcggctttcagtggagggcaggggtctggcacaggtgcgatggcaccccatgctccaggcatacagatgtggtttctcggctgcaccgggccaggctgcgggtgtgcaggcgtctgctaagttgtgtgatgtatcagcacaggctttgagacgtctggaccctgtccttcctcccgtgaggggttcttgttctttctgactcaggtgacttttcagcccttccaattcccctctttttctgccctcccctccaactcagccaacccaggtgtgggcagtcagggagggagggagtgtcccaccacgttctcagggcagcccttgactcctaagccccttcctccttccattctgcatcccctccccatccaacctaaatgccacagctggggctgagctgtattcctgtggagggacctctgccgtgcctctctgaggtcaggctgtgctgtgtgatgggcaggctttgccccagcccacccctggcaaggtgcacttgttttctggtttgtacaaggtgtcctgggggcccgtggcttccctgccagtgaggagtgacttctccctctcttccagtcctgtaggggagacaaaaccagattggggggcccaaggggagcatggaaaaggccggctcccctgtctttccttggctgtcagagtcagggtaacacacaccaagagtggagtgcggccagcaagtttgagacctgcccgccctcctcgcagctctgctctgtgtcctcaggaagtcacagagtctactgaggcaaggagagggtgattctttccccaaatcccttcttccctggttcccaaaccaaagacagcctgcagccctttctgcatggggtgctctgttgacaggcttcccagatccctgagtctctctttccttcctcctcgatctttagttgtccacggtcaattcagtgcttccattgggggacagtcccctccgggatgacctgattcacctccagcccagggaatggaatctagaggaatacgtggggtgggtctggacaaggagcggcaggaatcaccacccatctccagctgtggagccctgtggaggggaaggggaagcttggggttcagaggggactcttccaggagaggggtgcccagcggaggtaaagatgatagagggttgtggggggtctctagttgaatgttttggcccatgactttggaacatggctggcagcttccagcagaagtcacgctccccatcccccaggggacataggacctttttcctgcttcctggtcactttcaaagaactatttgcgcaatctgtgggtctgtggattcacggggctttctgtgtgggtgctgcagttgcttttgtctgcagcagcaggacacatctttcctcttactcagccctttatggcccatggggaactccgtggctcagggagagctgaactccaggggtgtgacctgggacgggtgggcctgaggtgcccagctcagggcagccaggtggctcatgggctgtagtgagccagctccctgggggaaaaggctgtgggccgttaggaccatcctccaggacaggtgacctctatgaggtcacctacggctgtggccgtgcaggcctccttccagcccagagtggcccagtagagcaaggcagacagtgacctccacccccgcagccctcttaaaaggccagtactcttgggggtggggggagggtttagaaagcatttgcccatctgcctttctttcccccagcccccacccgctttgaatgtagagacccgtgggcacttttccttttgtggtggggggtgcggaggaggtacccccacccctggcacagccgcctggaatgcaggactgtcactgctgttcgggtgatgacctcgttgccaagctcctcctgtccccttgttctgggggcaggcgctgtgcttctgtgaggtggtttagcttttgctttcgaagtggccagctgcggccaccaggtctcagcacaagagcgcttcctttgcacagaatgagcttcgagctttgttcagactaaatgaatgtatctgggaggggtcgggggcacgagttgattccaagcacatgcctttgctgagtgtgtgtgtgctgggagagtcagagtggatgtagagcgcggttttatttttgtactgacattggtaagagactgtatagcatctatttatttagatgatttatctggtaaatgaggcaaaaaaattattaaaaatacattaaagatgatttaaaaaaaagaaaa

SEQ ID NO: 8 - DNA, 3865 bp, Homo sapiens
tttttcaattttgaacattttgcaaaacgaggggttcgaggcaggtgagagcatcctgcacgtcgccggggagcccgcgggcacttggcgcgctctcctgggaccgtctgcactggaaacccgaaagtttttttttaatatatatttttatgcagatgtatttataaagatataagtaatttttttcttcccttttctccaccgccttgagagcgagtacttttggcaaaggacggaggaaaagctcagcaacattttagggggcggttgtttctttctttcttatttcttttttaaggggaaaaaatttgagtgcatcgcgatggagaaaatgtcccgaccgctccccctgaatcccacctttatcccgcctccctacggcgtgctcaggtccctgctggagaacccgctgaagctcccccttcaccacgaagacgcatttagtaaagataaagacaaagaaaagaagctggatgatgagagtaacagcccgacggtcccccagtcggcattcctggggcctaccttatgggacaaaacccttccctatgacggagatactttccagttggaatacatggacctggaggagtttttgtcagaaaatggcattccccccagcccatctcagcatgaccacagccctcaccctcctgggctgcagccagcttcctcggctgccccctcggtcatggacctcagcagccgggcctctgcaccccttcaccctggcatcccatctccgaactgtatgcagagccccatcagaccaggtcagctgttgccagcaaaccgcaatacaccaagtcccattgatcctgacaccatccaggtcccagtgggttatgagccagacccagcagatcttgccctttccagcatccctggccaggaaatgtttgaccctcgcaaacgcaagttctctgaggaagaactgaagccacagcccatgatcaagaaagctcgcaaagtcttcatccctgatgacctgaaggatgacaagtactgggcaaggcgcagaaagaacaacatggcagccaagcgctcccgcgacgcccggaggctgaaagagaaccagatcgccatccgggcctcgttcctggagaaggagaactcggccctccgccaggaggtggctgacttgaggaaggagctgggcaaatgcaagaacatacttgccaagtatgaggccaggcacgggcccctgtaggatggcatttttgcaggctggctttggaatagatggacagtttgtttcctgtctgatagcaccacacgcaaaccaacctttctgacatcagcactttaccagaggcataaacacaactgactcccattttggtgtgcatctgtgtgtgtgtgcgtgtatatgtgcttgtgctcatgtgtgtggtcagcggtatgtgcgtgtgcgtgttcctttgctcttgccattttaaggtagccctctcatcgtcttttagttccaacaaagaaaggtgccatgtctttactagactgaggagccctctcgcgggtctcccatcccctccctccttcactcctgcctcctcagctttgcttcatgttcgagcttacctactcttccaggactctctgcttggattcactaaaaagggccctggtaaaatagtggatctcagtttttaagagtacaagctcttgtttctgtttagtccgtaagttaccatgctaatgaggtgcacacaataacttagcactactccgcagctctagtcctttataagttgctttcctcttactttcagttttggtgataatcgtcttcaaattaaagtgctgtttagatttattagatcccatatttacttactgctatctactaagtttccttttaattctaccaaccccagataagtaagagtactattaatagaacacagagtgtgtttttgcactgtctgtacctaaagcaataatcctattgtacgctagagcatgctgcctgagtattactagtggacgtaggatattttccctacctaagaatttcactgtcttttaaaaaacaaaaagtaaagtaatgcatttgagcatggccagactattccctaggacaaggaagcagagggaaatgggaggtctaaggatgaggggttaatttatcagtacatgagccaaaaactgcgtcttggattagcctttgacattgatgtgttcggttttgttgttccccttccctcacaccctgcctcgcccccacttttctagttaactttttccatatccctcttgacattcaaaacagttacttaagattcagttttcccactttttggtaatatatatatttttgtgaattatactttgttgtttttaaaaagaaaatcagttgattaagttaataagttgatgttttctaaggccctttttcctagtggtgtcatttttgaatgcctcataaattaatgattctgaagcttatgtttcttattctctgtttgcttttgaacgtatgtgctcttataaagtggacttctgaaaaatgaatgtaaaagacactggtgtatctcagaaggggatggtgttgtcacaaactgtggttaatccaatcaatttaaatgtttactatagaccaaaaggagagattattaaatcgtttaatgtttatacagagtaattataggaagttcttttttgtacagtatttttcagatataaatactgacaatgtattttggaagacatatattatatatagaaaagaggagaggaaaactattccatgttttaaaattatatagcaaagatatatattcaccaatgttgtacagagaagaagtgcttgggggtttttgaagtctttaatattttaagccctatcactgacacatcagcatgttttctgctttaaattaaaattttatgacagtatcgaggcttgtgatgacgaatcctgctctaaaatacacaaggagctttcttgtttcttattaggcctcagaaagaagtcagttaacgtcacccaaaagcacaaaatggattttagtcaaatatttattggatgatacagtgttttttaggaaaagcatctgccacaaaaatgttcacttcgaaattctgagttcctggaatggcacgttgctgccagtgccccagacagttcttttctaccctgcgggcccgcacgttttatgaggttgatatcggtgctatgtgtttggtttataatttgatagatgtttgactttaaagatgattgttcttttgtttcattaagttgtaaaatgtcaagaaattctgctgttacgacaaagaaacattttacgctagattaaaatatcctttcatcaatgggattttctagtttcctgccttcagagtatctaatcctttaatgatctggtggtctcctcgtcaatccatcagcaatgcttctctcatagtgtcatagacttgggaaacccaaccagtaggatatttctacaaggtgttcattttgtcacaagctgtagataacagcaagagatgggggtgtattggaattgcaatacattgttcaggtgaataataaaatcaaaaacttttgcaatcttaagcagagataaataaaagatagcaatatgagacacaggtggacgtagagttggcctttttacaggcaaagaggcgaattgtagaattgttagatggcaatagtcattaaaaacatagaaaaatgatgtctttaagtggagaattgtggaaggattgtaacatggaccatccaaatttatggccgtatcaaatggtagctgaaaaaactatatttgagcactggtctctcttggaattagatgtttatatcaaatgagcatctcaaatgttttctgcagaaaaaaataaaaagattctaataaaaaaa

SEQ ID NO: 9 - DNA, 2656 bp, Homo sapiens
cacacctgatggtgtgactcggccgacgcgagcgccgcgcttcgcttcagctgctagctggcccaagggaggcgaccgcggagggtggcgaggggcggccaggacccgcagccccggggccgggccggtccggaccgccagggagggcaggtcagtgggcagatcgcgtccgcgggattcaatctctgcccgctctgataacagtccttttccctggcgctcacttcgtgcctggcacccggctgggcgcctcaagaccgttgtctcttcgatcgcttctttggacttggcgaccatttcagagatgtcttccagaagtaccaaagatttaattaaaagtaagtggggatcgaagcctagtaactccaaatccgaaactacattagaaaaattaaagggagaaattgcacacttaaagacatcagtggatgaaatcacaagtgggaaaggaaagctgactgataaagagagacacagacttttggagaaaattcgagtccttgaggctgagaaggagaagaatgcttatcaactcacagagaaggacaaagaaatacagcgactgagagaccaactgaaggccagatatagtactaccgcattgcttgaacagctggaagagacaacgagagaaggagaaaggagggagcaggtgttgaaagccttatctgaagagaaagacgtattgaaacaacagttgtctgctgcaacctcacgaattgctgaacttgaaagcaaaaccaatacactccgtttatcacagactgtggctccaaactgcttcaactcatcaataaataatattcatgaaatggaaatacagctgaaagatgctctggagaaaaatcagcagtggctcgtgtatgatcagcagcgggaagtctatgtaaaaggacttttagcaaagatctttgagttggaaaagaaaacggaaacagctgctcattcactcccacagcagacaaaaaagcctgaatcagaaggttatcttcaagaagagaagcagaaatgttacaacgatctcttggcaagtgcaaaaaaagatcttgaggttgaacgacaaaccataactcagctgagttttgaactgagtgaatttcgaagaaaatatgaagaaacccaaaaagaagttcacaatttaaatcagctgttgtattcacaaagaagggcagatgtgcaacatctggaagatgataggcataaaacagagaagatacaaaaactcagggaagagaatgatattgctaggggaaaacttgaagaagagaagaagagatccgaagagctcttatctcaggtccagtttctttacacatctctgctaaagcagcaagaagaacaaacaagggtagctctgttggaacaacagatgcaggcatgtactttagactttgaaaatgaaaaactcgaccgtcaacatgtgcagcatcaattgcatgtaattcttaaggagctccgaaaagcaagaaatcaaataacacagttggaatccttgaaacagcttcatgagtttgccatcacagagccattagtcactttccaaggagagactgaaaacagagaaaaagttgccgcctcaccaaaaagtcccactgctgcactcaatgaaagcctggtggaatgtcccaagtgcaatatacagtatccagccactgagcatcgcgatctgcttgtccatgtggaatactgttcaaagtagcaaaataagtatttgttttgatattaaaagattcaatactgtattttctgttagcttgtgggcattttgaattatatatttcacattttgcataaaactgcctatctacctttgacactccagcatgctagtgaatcatgtatcttttaggctgctgtgcatttctcttggcagtgatacctccctgacatggttcatcatcaggctgcaatgacagaatgtggtgagcagcgtctactgagactactaacattttgcactgtcaaaatacttggtgaggaaaagatagctcaggttattgctaatgggttaatgcaccagcaagcaaaatattttatgttttgggggttttgaaaaatcaaagataattaaccaaggatcttaactgtgttcgcattttttatccaagcacttagaaaacctacaatcctaattttgatgtccattgttaagaggtggtgatagatactattttttttttcatattgtatagcggttattagaaaagttggggattttcttgatctttattgctgcttaccattgaaacttaacccagctgtgttccccaactctgttctgcgcacgaaacagtatctgtttgaggcataatcttaagtggccacacacaatgttttctcttatgttatctggcagtaactgtaacttgaattacattagcacattctgcttagctaaaattgttaaaataaactttaataaacccatgtagccctctcatttgattgacagtattttagttatttttggcattcttaaagctgggcaatgtaatgatcagatctttgtttgtctgaacaggtatttttatacatgctttttgtaaaccaaaaacttttaaatttcttcaggttttctaacatgcttaccactgggctactgtaaatgagaaaagaataaaattatttaatgttttaaaaaaaaaaaaaaaaaaaa

SEQ ID NO: 10 - DNA, 3685 bp, Homo sapiens
agtggactcacgcaggcgcaggagactacacttcccaggaactccgggccgcgttgttcgctggtacctccttctgacttccggtattgctgcggtctgtagggccaatcgggagcctggaattgctttcccggcgctctgattggtgcattcgactaggctgcctgggttcaaaatttcaacgatactgaatgagtcccgcggcgggttggctcgcgcttcgttgtcagatctgaggcgaggctaggtgagccgtgggaagaaaagagggagcagctagggcgcgggtctccctcctcccggagtttggaacggctgaagttcaccttccagcccctagcgccgttcgcgccgctaggcctggcttctgaggcggttgcggtgctcggtcgccgcctaggcggggcagggtgcgagcaggggcttcgggccacgcttctcttggcgacaggattttgctgtgaagtccgtccgggaaacggaggaaaaaaagagttgcgggaggctgtcggctaataacggttcttgatacatatttgccagacttcaagatttcagaaaaggggtgaaagagaagattgcaactttgagtcagacctgtaggcctgatagactgattaaaccacagaaggtgacctgctgagaaaagtggtacaaatactgggaaaaacctgctcttctgcgttaagtgggagacaatgtcacaagttaaaagctcttattcctatgatgccccctcggatttcatcaatttttcatccttggatgatgaaggagatactcaaaacatagattcatggtttgaggagaaggccaatttggagaataagttactggggaagaatggaactggagggctttttcagggcaaaactcctttgagaaaggctaatcttcagcaagctattgtcacacctttgaaaccagttgacaacacttactacaaagaggcagaaaaagaaaatcttgtggaacaatccattccgtcaaatgcttgttcttccctggaagttgaggcagccatatcaagaaaaactccagcccagcctcagagaagatctcttaggctttctgctcagaaggatttggaacagaaagaaaagcatcatgtaaaaatgaaagccaagagatgtgccactcctgtaatcatcgatgaaattctaccctctaagaaaatgaaagtttctaacaacaaaaagaagccagaggaagaaggcagtgctcatcaagatactgctgaaaagaatgcatcttccccagagaaagccaagggtagacatactgtgccttgtatgccacctgcaaagcagaagtttctaaaaagtactgaggagcaagagctggagaagagtatgaaaatgcagcaagaggtggtggagatgcggaaaaagaatgaagaattcaagaaacttgctctggctggaatagggcaacctgtgaagaaatcagtgagccaggtcaccaaatcagttgacttccacttccgcacagatgagcgaatcaaacaacatcctaagaaccaggaggaatataaggaagtgaactttacatctgaactacgaaagcatccttcatctcctgcccgagtgactaagggatgtaccattgttaagcctttcaacctgtcccaaggaaagaaaagaacatttgatgaaacagtttctacatatgtgccccttgcacagcaagttgaagacttccataaacgaacccctaacagatatcatttgaggagcaagaaggatgatattaacctgttaccctccaaatcttctgtgaccaagatttgcagagacccacagactcctgtactgcaaaccaaacaccgtgcacgggctgtgacctgcaaaagtacagcagagctggaggctgaggagctcgagaaattgcaacaatacaaattcaaagcacgtgaacttgatcccagaatacttgaaggtgggcccatcttgcccaagaaaccacctgtgaaaccacccaccgagcctattggctttgatttggaaattgagaaaagaatccaggagcgagaatcaaagaagaaaacagaggatgaacactttgaatttcattccagaccttgccctactaagattttggaagatgttgtgggtgttcctgaaaagaaggtacttccaatcaccgtccccaagtcaccagcctttgcattgaagaacagaattcgaatgcccaccaaagaagatgaggaagaggacgaaccggtagtgataaaagctcaacctgtgccacattatggggtgccttttaagccccaaatcccagaggcaagaactgtggaaatatgccctttctcgtttgattctcgagacaaagaacgtcagttacagaaggagaagaaaataaaagaactgcagaaaggggaggtgcccaagttcaaggcacttcccttgcctcattttgacaccattaacctgccagagaagaaggtaaagaatgtgacccagattgaacctttctgcttggagactgacagaagaggtgctctgaaggcacagacttggaagcaccagctggaagaagaactgagacagcagaaagaagcagcttgtttcaaggctcgtccaaacaccgtcatctctcaggagccctttgttcccaagaaagagaagaaatcagttgctgagggcctttctggttctctagttcaggaaccttttcagctggctactgagaagagagccaaagagcggcaggagctggagaagagaatggctgaggtagaagcccagaaagcccagcagttggaggaggccagactacaggaggaagagcagaaaaaagaggagctggccaggctacggagagaactggtgcataaggcaaatccaatacgcaagtaccagggtctggagataaagtcaagtgaccagcctctgactgtgcctgtatctcccaaattctccactcgattccactgctaaactcagctgtgagctgcggataccgcccggcaatgggacctgctcttaacctcaaacctaggaccgtcttgctttgtcattgggcatggagagaacccatttctccagacttttacctacccgtgcctgagaaagcatacttgacaactgtggactccagttttgttgagaattgttttcttacattactaaggctaataatgagatgtaactcatgaatgtctcgattagactccatgtagttacttcctttaaaccatcagccggccttttatatgggtcttcactctgactagaatttagtctctgtgtcagcacagtgtaatctctattgctattgccccttacgactctcaccctctccccactttttttaaaaattttaaccagaaaataaagatagttaaatcctaagatagagattaagtcatggtttaaatgaggaacaatcagtaaatcagattctgtcctcttctctgcataccgtgaatttatagttaaggatccctttgctgtgagggtagaaaacctcaccaactgcaccagtgaggaagaagactgcgtggattcatggggagcctcacagcagccacgcagcaggctctgggtggggctgccgttaaggcacgttctttccttactggtgctgataacaacagggaaccgtgcagtgtgcattttaagacctggcctggaataaatacgttttgtctttccctcaaaaaaaaaaaaaaaaaaaaaaa

SEQ ID NO: 11 - DNA, 3670 bp, Homo sapiens
ccggtttgttagggagtcgtgtacgtgccttggtcgcttctgtagctccgagggcaggttgcggaagaaagcccaggcggtctgtggcccagaggaaaggcctgcagcaggacgaggacctgagccaggaatgcaggatggcggcggtgaagaaggaagggggtgctctgagtgaagccatgtccctggagggagatgaatgggaactgagtaaagaaaatgtacaacctttaaggcaagggcggatcatgtccacgcttcagggagcactggcacaagaatctgcctgtaacaatactcttcagcagcagaaacgggcatttgaatatgaaattcgattttacactggaaatgaccctctggatgtttgggataggtatatcagctggacagagcagaactatcctcaaggtgggaaggagagtaatatgtcaacgttattagaaagagctgtagaagcactacaaggagaaaaacgatattatagtgatcctcgatttctcaatctctggcttaaattagggcgtttatgcaatgagcctttggatatgtacagttacttgcacaaccaagggattggtgtttcacttgctcagttctatatctcatgggcagaagaatatgaagctagagaaaactttaggaaagcagatgcgatatttcaggaagggattcaacagaaggctgaaccactagaaagactacagtcccagcaccgacaattccaagctcgagtgtctcggcaaactctgttggcacttgagaaagaagaagaggaggaagtttttgagtcttctgtaccacaacgaagcacactagctgaactaaagagcaaagggaaaaagacagcaagagctccaatcatccgtgtaggaggtgctctcaaggctccaagccagaacagaggactccaaaatccatttcctcaacagatgcaaaataatagtagaattactgtttttgatgaaaatgctgatgaggcttctacagcagagttgtctaagcctacagtccagccatggatagcaccccccatgcccagggccaaagagaatgagctgcaagcaggcccttggaacacaggcaggtccttggaacacaggcctcgtggcaatacagcttcactgatagctgtacccgctgtgcttcccagtttcactccatatgtggaagagactgcacgacagccagttatgacaccatgtaaaattgaacctagtataaaccacatcctaagcaccagaaagcctggaaaggaagaaggagatcctctacaaagggttcagagccatcagcaagcgtctgaggagaagaaagagaagatgatgtattgtaaggagaagatttatgcaggagtaggggaattctcctttgaagaaattcgggctgaagttttccggaagaaattaaaagagcaaagggaagccgagctattgaccagtgcagagaagagagcagaaatgcagaaacagattgaagagatggagaagaagctaaaagaaatccaaactactcagcaagaaagaacaggtgatcagcaagaagagacgatgcctacaaaggagacaactaaactgcaaattgcttccgagtctcagaaaataccaggaatgactctatccagttctgtttgtcaagtaaactgttgtgccagagaaacttcacttgcggagaacatttggcaggaacaacctcattctaaaggtcccagtgtacctttctccatttttgatgagtttcttctttcagaaaagaagaataaaagtcctcctgcagatcccccacgagttttagctcaacgaagaccccttgcagttctcaaaacctcagaaagcatcacctcaaatgaagatgtgtctccagatgtttgtgatgaatttacaggaattgaacccttgagcgaggatgccattatcacaggcttcagaaatgtaacaatttgtcctaacccagaagacacttgtgactttgccagagcagctcgttttgtatccactccttttcatgagataatgtccttgaaggatctcccttctgatcctgagagactgttaccggaagaagatctagatgtaaagacctctgaggaccagcagacagcttgtggcactatctacagtcagactctcagcatcaagaagctgagcccaattattgaagacagtcgtgaagccacacactcctctggcttctctggttcttctgcctcggttgcaagcacctcctccatcaaatgtcttcaaattcctgagaaactagaacttactaatgagacttcagaaaaccctactcagtcaccatggtgttcacagtatcgcagacagctactgaagtccctaccagagttaagtgcctctgcagagttgtgtatagaagacagaccaatgcctaagttggaaattgagaaggaaattgaattaggtaatgaggattactgcattaaacgagaatacctaatatgtgaagattacaagttattctgggtggcgccaagaaactctgcagaattaacagtaataaaggtatcttctcaacctgtcccatgggacttttatatcaacctcaagttaaaggaacgtttaaatgaagattttgatcatttttgcagctgttatcaatatcaagatggctgtattgtttggcaccaatatataaactgcttcacccttcaggatcttctccaacacagtgaatatattacccatgaaataacagtgttgattatttataaccttttgacaatagtggagatgctacacaaagcagaaatagtccatggtgacttgagtccaaggtgtctgattctcagaaacagaatccacgatccctatgattgtaacaagaacaatcaagctttgaagatagtggacttttcctacagtgttgaccttagggtgcagctggatgtttttaccctcagcggctttcggactgtacagatcctggaaggacaaaagatcctggctaactgttcttctccctaccaggtagacctgtttggtatagcagatttagcacatttactattgttcaaggaacacctacaggtcttctgggatgggtccttctggaaacttagccaaaatatttctgagctaaaagatggtgaattgtggaataaattctttgtgcggattctgaatgccaatgatgaggccacagtgtctgttcttggggagcttgcagcagaaatgaatggggtttttgacactacattccaaagtcacctgaacaaagccttatggaaggtagggaagttaactagtcctggggctttgctctttcagtgagctaggcaatcaagtctcacagattgctgcctcagagcaatggttgtattgtggaacactgaaactgtatgtgctgtaatttaatttaggacacatttagatgcactaccattgctgttctactttttggtacaggtatattttgacgtcactgatattttttatacagtgatatacttactcatggccttgtctaacttttgtgaagaactattttattctaaacagactcattacaaatggttaccttgttatttaacccatttgtctctacttttccctgtacttttcccatttgtaatttgtaaaatgttctcttatgatcaccatgtattttgtaaataataaaatagtatctgttaaaaaaaaaaaaaaaaaaa

SEQ ID NO: 12 - DNA, 4504 bp, Homo sapiens
gggcagtccgcactgcaattggttggcgtctccgggacggatttgaaacttggcggttaaagctccggctgggacagggcggcgggaggcccagggagaacggggaagggacatttagtttgagacggtgctgagataggatcatgaaggaagaggtgaagggaattcctgtaagagtggcgctgcgttgtcgccctctggtccccaaagagattagcgagggctgccagatgtgcctttccttcgtgcccggagagcctcaggtggtggttggtacagataaatccttcacctacgattttgtatttgatccctctactgaacaggaagaagtcttcaatacagcagtagcgccactcataaaaggtgtatttaaaggatataatgcaacggtcctggcctatgggcagactggctctggaaaaacctattcaatgggaggtgcatatactgcagagcaagagaatgaaccaacagttggggttattcctagggtaatacaactgctcttcaaagaaattgataaaaagagtgactttgaatttactctgaaagtgtcttacttagagatttacaatgaagaaattttggatcttctatgcccatctcgtgagaaagctcaaataaatatacgagaggatcctaaggaaggcataaagattgtgggactcactgagaagactgttttggttgccttggatactgtttcctgtttggaacagggcaacaactctaggactgtggcctccacggctatgaactcccagtcgtcccgatctcatgccatctttacaatctccttagagcaaagaaagaaaagtgacaagaatagcagctttcgctccaagctgcatcttgtagacctcgctggatcagaaagacagaagaaaaccaaggctgaaggggatcgtctaaaagagggtattaatattaaccgaggcctcctatgcttgggaaatgtaatcagtgctcttggagatgacaaaaagggtggctttgtgccctacagagattccaagttgactcgactgcttcaagattctctaggaggtaatagccatactcttatgatagcctgtgtgagtcctgctgactccaatctagaggaaacattaaatacccttcgctatgctgacagagcaagaaaaatcaagaacaaacctattgttaatattgatccccagacagctgaacttaatcatctaaagcaacaggtacaacagctacaagtcttgttgctacaggcccatggaggtaccctgcctggatctataactgtggaaccatcagagaatctacaatccctgatggagaagaatcagtccctggtagaggagaatgaaaaattaagtcgtggtctgagcgaggcagctggtcagacagcccagatgttggagaggatcattttgacagagcaagcgaatgaaaaaatgaacgccaagctagaagagctcaggcagcatgcggcctgcaaactggatcttcaaaagctagtggagactttggaagaccaggaattgaaagaaaatgtagagataatttgtaacctgcagcaattgattacccagttatcggatgaaactgttgcttgcatggctgcagccattgatactgcggtggagcaagaagcccaagtagaaaccagtccagagacgagcaggtcttctgacgcttttaccactcagcatgctctccgtcaagcgcagatgtctaaggagctggttgagttgaataaagcgcttgcactgaaagaggccctggctaggaagatgactcagaatgacagccaactgcagcccattcagtaccaataccaggataacataaaagagctagaattagaagtcatcaatctgcaaaaggaaaaggaagaattggttcttgaacttcagacagcaaagaaggatgccaaccaagccaagttgagtgagcgccgccgcaaacgtctccaggagctggagggtcaaattgctgatctgaagaagaaactgaatgagcagtccaaacttctgaaactaaaggaatccacagagcgtactgtctccaaactgaaccaggagatacggatgatgaaaaaccagcgggtacagttaatgcgtcaaatgaaagaagatgctgagaagtttagacagtggaagcagaaaaaagacaaagaagtaatacagttaaaagaacgagaccgtaagaggcaatatgagctgctgaaacttgaaagaaacttccagaaacaatccaatgtgctcagacgtaaaacggaggaggcagcagctgccaacaagcgtctcaaggatgctctccagaaacaacgggaggttgcagataagcggaaagagactcagagccgtggaatggaaggcactgcagctcgagtgaagaattggcttggaaacgaaattgaggttatggtcagtactgaggaagccaaacgccatctgaatgacctccttgaagatagaaagatcctggctcaagatgtggctcaactcaaagaaaaaaaggaatctggggagaatccacctcctaaactccggaggcgtacattctcccttactgaagtgcgtggtcaagtttcggagtcagaagattctattacaaagcagattgaaagcctagagactgaaatggaattcaggagtgctcagattgctgacctacagcagaagctgctggatgcagaaagtgaagacagaccaaaacaacgctgggagaatattgccaccattctggaagccaagtgtgccctgaaatatttgattggagagctggtctcctccaaaatacaggtcagcaaacttgaaagcagcctgaaacagagcaagaccagctgtgctgacatgcagaagatgctgtttgaggaacgaaatcattttgccgagatagagacagagttacaagctgagctggtcagaatggagcaacagcaccaagagaaggtgctgtaccttctcagccagctgcagcaaagccaaatggcagagaagcagttagaggaatcagtcagtgaaaaggaacagcagctgctgagcacactgaagtgtcaggatgaagaacttgagaaaatgcgagaagtgtgtgagcaaaatcagcagcttctccgagagaatgaaatcatcaagcagaaactgaccctcctccaggtagccagcagacagaaacatcttcctaaggatacccttctatctccagactcttcttttgaatatgtcccacctaagccaaaaccttctcgtgttaaagaaaagttcctggagcaaagcatggacatcgaggatctaaaatattgttcagagcattctgtgaatgagcatgaggatggtgatggtgatgatgatgagggggatgacgaggaatggaagccaacaaaattagttaaggtgtccaggaagaacatccaagggtgttcctgcaagggctggtgtggaaacaagcagtgtgggtgcaggaagcaaaagtcagactgtggtgtggactgttgctgtgaccccacaaagtgtcggaaccgccagcaaggcaaggatagcttgggcactgttgaacggacccaggattccgaaggctccttcaaactggaggatcctaccgaggtgaccccaggattgagcttctttaatcccgtctgtgccacccccaatagcaagatcctgaaagagatgtgcgatgtggagcaggtgctgtcaaagaagactcccccagctccctccccttttgacctcccagagttgaaacatgtagcaacagaataccaagaaaacaaggctccagggaagaaaaagaaacgggctctggccagcaacaccagcttcttctctggctgctcccctatcgaagaagaggcccactgaagttggagtcatcatctctacccccagtctggcttgggagatgctttcaggttgcagccagaaggggttttttaaatgacttctctggatttcaggtttcttgctgttgaaaaaaggaacaaagcgttactgaaaagaaggtaacctttgttggatgtgggccttagcctccaggtccagactactactctatgttctccagaagggtgctaagtcacctactgaagagagaaccaactgactttcctattgactcatcaggaaccagtcctcagtctggtcaagttgtttcttatttgtgagcagttcaggctatctcctgatggggatgaggccaaggctttcttatcttttggttgtctctgcttaatggaggagcctggcctaggatggaggcctggcttagatctttcattccacctcaggaatgaggttgtgatctttcctgtcctgaccctctctgaattatgtttcaatagtactcttgattgtctgccatgttgttgaagcaaatgaattatttttaaatgttaagtaagtaaataaaccttagcccgtctactgtttgggaagatccttctgtgctagagggagaaataaaatttcaacctgtgttcctcagcccctgaggaagctattaaggggattcattacaagtaaaa

SEQ ID NO: 13 - DNA, 1566 bp, Homo sapiens
gggccgcccaatggggcgcaagcgacgcggtatttgaatcctggaacaaggctacagcgtcgaagatccccagcgctgcgggctcggagagcagtcctaacggcgcctcgtacgctagtgtcctcccttttcagtccgcgtccctccctgggccgggctggcactcttgccttccccgtccctcatggcgctgctccgacgcccgacggtgtccagtgatttggagaatattgacacaggagttaattctaaagttaagagtcatgtgactattaggcgaactgttttagaagaaattggaaatagagttacaaccagagcagcacaagtagctaagaaagctcagaacaccaaagttccagttcaacccaccaaaacaacaaatgtcaacaaacaactgaaacctactgcttctgtcaaaccagtacagatggaaaagttggctccaaagggtccttctcccacacctgaggatgtctccatgaaggaagagaatctctgccaagctttttctgatgccttgctctgcaaaatcgaggacattgataacgaagattgggagaaccctcagctctgcagtgactacgttaaggatatctatcagtatctcaggcagctggaggttttgcagtccataaacccacatttcttagatggaagagatataaatggacgcatgcgtgccatcctagtggattggctggtacaagtccactccaagtttaggcttctgcaggagactctgtacatgtgcgttggcattatggatcgatttttacaggttcagccagtttcccggaagaagcttcaattagttgggattactgctctgctcttggcttccaagtatgaggagatgttttctccaaatattgaagactttgtttacatcacagacaatgcttataccagttcccaaatccgagaaatggaaactctaattttgaaagaattgaaatttgagttgggtcgacccttgccactacacttcttaaggcgagcatcaaaagccggggaggttgatgttgaacagcacactttagccaagtatttgatggagctgactctcatcgactatgatatggtgcattatcatccttctaaggtagcagcagctgcttcctgcttgtctcagaaggttctaggacaaggaaaatggaacttaaagcagcagtattacacaggatacacagagaatgaagtattggaagtcatgcagcacatggccaagaatgtggtgaaagtaaatgaaaacttaactaaattcatcgccatcaagaataagtatgcaagcagcaaactcctgaagatcagcatgatccctcagctgaactcaaaagccgtcaaagaccttgcctccccactgataggaaggtcctaggctgccgtggcccctggggatgtgtgcttcattgtgccctttttcttattggtttagaactcttgattttgtacatagtcctctggtctatctcatgaaacctcttctcagaccagttttctaaacatatattgaggaaaaataaagcgattggtttttcttaaggtaaaaaaaaaaaaaaaaaa

SEQ ID NO: 14 - DNA, 7293 bp, Homo sapiens
ctggggagccggcgctggaggtggtgagtggcgtggggactgtgtcgagggggtccccaaggtgccggaccctgcggaggggcgaagtttcggcactggggagggcgtgcggacgctttccctacaggcgaccactgctctgcgggcgggtggtcttagctccagtcccccattcagttcctcagcattccaggtcggcggcgaaggggtccccgaacgaagggcgcaaggcagcgtctctgctgggaccgggaagccggacttcagggcctctcggcccgtgggcttctccccgagtctccccgagtcggttggcattaagagtttagcagatactttcagaaatggatacataagaaatggctggaaatcaaatgaatgtccaaagaagagcttagggtcttagtaacattcttttttaaaataactgtctgccaaaatgtcattacacagtactcataatagaaataacagcggtgatattcttgatattccttcttcccaaaatagttcatcactgaatgccctcacccacagtagccgacttaagctgcatttgaagtcggatatgtcagaatgtgaaaatgatgatccattattgagatctgcaggtaaagtcagagacataaatagaacttatgttatttctgccagtagaaaaacagcagacatgccccttacccctaatcctgtaggtagattggcacttcagaggagaactacaaggaacaaagaatcatctttgcttgttagtgagttggaagacacaactgaaaaaacagcagaaacacgtcttacattacaacgtcgtgctaaaacagattctgcagaaaagtggaaaacagctgaaatagattctgtcaaaatgacactgaatgtgggaggtgaaacagaaaataatggtgtttctaaggaaagtagaacaaatgtaaggattgtaaataatgctaaaaactcttttgttgcctcttctgtacctttagatgaagatccacaggtcattgaaatgatggctgataagaaatacaaagaaacattttctgcccccagtagagcaaatgaaaatgttgcacttaagtactcaagtaatagaccacccattgcttccctgagtcagactgaagttgttagatcaggacacttgacaacgaaacctactcagagcaagttggatatcaaagtgttgggaacaggaaacttgtatcatagaagtattgggaaggaaattgcaaaaacttcaaataaatttgggagcttagaaaaaagaacacctacaaaatgtacaacagaacacaaactgacaacaaagtgcagcctgcctcagcttaagagcccagctccatcaatactgaagaatagaatgtctaaccttcaagttaaacaaagaccaaaaagttcctttcttgcaaataaacaggaaagatccgcagaaaatacaattcttcccgaagaagaaactgtagttcagaacacctctgcaggaaaagaccccttaaaagtagagaatagtcaagtgacagtggcagtacgcgtaagacctttcaccaagagagagaagattgaaaaagcatcccaggtagtcttcatgagtgggaaagaaataactgtggaacaccctgacacgaaacaagtttataattttatttatgatgtttcattctggtcttttgatgaatgtcatcctcactacgctagccagacaactgtctatgagaagctagcagcaccactcctagaaagagccttcgaaggcttcaatacctgtctttttgcttatggtcagactggctctggaaaatcatatacgatgatgggatttagtgaagaaccaggaataattccaagattttgtgaagatcttttttctcaagtagccagaaaacaaacccaagaggtcagctatcacattgaaatgagcttctttgaagtatataatgaaaaaattcacgaccttctggtttgtaaagatgaaaatgggcagagaaagcaaccactgagagtgagggaacatcctgtttatggaccatatgttgaagcactgtcaatgaacattgtcagttcttacgctgatatccagagttggctagaattgggaaataaacaaagagctactgctgctactggtatgaatgataaaagttcccgatctcattcagttttcaccctggtgatgacccagaccaagacagaatttgtggaaggggaagaacacgatcacagaataacaagtcgaattaacctaatagatctggcaggcagtgagcgctgctctacggctcacactaatggagatcgactaaaggaaggtgtgagtattaataagtccttgctaactttgggaaaagttatatctgcactttcggaacaagcaaaccaaaggagtgtttttattccttatcgtgaatctgttcttacatggctgttaaaagaaagtctgggtggaaattcaaaaactgcaatgattgctacgattagtcccgctgccagcaacatagaagaaacattaagcacacttagatatgctaaccaagcccgtttaatagtcaacattgctaaagtaaatgaagatatgaacgctaagttaattagagaattgaaggcagaaattgcaaagctaaaagctgctcagagaaacagtcggaatattgaccctgaacgatacaggctctgtcggcaagaaataacatccttaagaatgaaactgcatcaacaggagagagacatggcagaaatgcaaagagtgtggaaagaaaagtttgaacaagctgaaaaaagaaaacttcaagaaacaaaagagttacagaaagcaggaattatgtttcaaatggacaatcatttaccaaaccttgttaatctgaatgaagatccacaactatctgagatgctgctatatatgataaaagaaggaacaactacagttggaaagtataaaccaaactcaagccatgatattcagttatctggggtgctgattgctgatgatcattgtactatcaaaaattttggtgggacagtgagtattatcccagttggggaagcaaagacatatgtaaatggaaaacatattttggaaatcacagtattacgtcatggtgatcgagtgattcttggtggagatcattattttagatttaatcatccagtagaagtccagaaaggaaaaaggccatctggaagagatactcctataagtgagggtccaaaagactttgaatttgcaaaaaatgagttgctcatggcacagagatcacaacttgaagcagaaataaaagaggctcagttgaaggcaaaggaagaaatgatgcaaggaatccagattgcaaaagaaatggctcagcaagagctttcttctcaaaaagctgcatatgaaagcaaaataaaagcactggaagcagaactgagagaagagtctcaaaggaaaaaaatgcaggaaataaataaccagaaggctaatcacaaaattgaggaattagaaaaggcaaagcagcatcttgaacaggaaatatatgtcaacaaaaagcgattagaaatggaaacattggctacaaaacaggctttagaagaccatagcatccgccatgcaagaattctggaagctttagaaactgaaaagcaaaaaattgctaaagaagtacaaattctacagcagaatcggaataatagggataaaacttttacagtgcagacaacttggagctctatgaaactctcaatgatgattcaggaagccaatgctatcagcagcaaattgaaaacatactatgtttttggcagacatgatatatcagataaaagtagttctgacacttctattcgggttcgtaacctgaaactaggaatctcaacattctggagtctggaaaagtttgaatctaaacttgcagcaatgaaagaactttatgagagtaatggtagtaacaggggtgaagatgccttttgtgatcctgaagatgaatgggaacccgacattacagatgcaccagtttcttcactttctagaaggaggagtaggagtttgatgaagaacagaagaatttctggttgtttacatgacatacaagtccatccaattaagaatttgcattcttcacattcatcaggtttaatggacaaatcaagcactatttactcaaattcagcagagtcctttcttcctggaatttgcaaagaattgattggttcttcgttagatttttttggacagagttatgatgaagaaagaactatagcagacagcctaattaatagttttcttaaaatttataatgggctatttgccatttccaaggctcatgaagaacaagatgaagaaagtcaagataacttgttttcttctgatcgagcaatccagtcacttactattcagactgcatgtgcttttgagcagctagtagtgctaatgaaacactggctgagtgatttactgccttgtaccaacatagcaagacttgaggatgagttgagacaagaagttaaaaaactgggaggctacttacagttatttttgcagggatgctgtttggatatttcatcaatgataaaagaggctcaaaagaatgcaatccaaattgtacaacaagctgtaaagtatgtggggcagttagcagttctgaaagggagcaagctacattttctagaaaacggtaacaataaagctgccagtgtccaggaggaattcatggatgctgtttgtgatggtgtaggcttaggaatgaagattttattagattctggactggaaaaagcaaaagaacttcagcatgaactctttaggcagtgtacaaaaaatgaggttaccaaagaaatgaaaactaatgccatgggattgattagatctcttgaaaacatctttgctgaatcgaaaattaaaagtttcagaaggcaagtacaagaagaaaactttgaataccaagatttcaagaggatggttaatcgtgctccagaattcttaaagttaaaacattgcttagagaaagctattgaaattattatttctgcactgaaaggatgccatagtgatataaatcttctccagacttgtgttgaaagtattcgcaacttggccagtgatttttacagtgacttcagtgtgccttctacttctgttggcagctatgagagtagagtaactcacattgtccaccaggaactagaatctctagctaagtctctcctcttttgttttgaatctgaagaaagccctgatttgttgaaaccctgggaaacttataatcaaaataccaaagaagaacaccaacaatctaaatcaagcgggattgacggcagtaagaataaaggtgtaccaaagcgtgtctatgagctccatggctcatccccagcagtgagctcagaggaatgcacacccagtaggattcagtgggtgtgaatactgatgtgtaggcacttttatgaccacccatgaaagaaaaagaacacttgctcggtaattttctttatgcaggagagtttaagagaaatcagcacagatatttcaaaaaagtccatgtctttttatctttaaaatatctatttatcaaaggccagacacagtggctcacgcctgtaatcccagcactttgggaggcgggcagatcacaaggtcaggagtttgagaccggcctggccaacatggcgaaaccccgtctctactaaaaatacaaaaatttgctgggcatggtggcgcgtgcctgtaatcccagctactaggggggctgaggcaggaggatcgcttgaacctgagaggcagaggttgcagtgagccaagatcatgccactttactccagtctgagcaacagaacgagacttagtcaaaataaataaataaataaataaataaataaataaataaataaataaaatatatttttatctttaaagtgtttaacattggtatactgtctgtagttggttcattagtcgtttataaagggttattttctcatgagtggaaacctgaacaatcagttacctttgtgcctatgccttctctctcctcagacagctgggatgtttatggtgaaatggcctgtacaagtttaactaagacaacttaacttgcattgttaatcaaaaattcttttctcaaagggttaactggttgccattttgaatagtatgttcaagggtgtagcttcctgtttctttccaaattataagtagctacctaaatatagtataattatatattaataatatggcttgctggcacagtagtttaccctgttatctgtgtttcataatgggggctgtatgaatattatttaaaactaataaaatgttgccagaattatactaaactgttggatgagattaggagatcagaggctggaccttctcttgataatgcttgttttgttaaaggtataatgaaataatttgtatatgatttgatgaagattaaagacccttattttccacagctttaaaaaaaaacctttatttatgatcaagtaataaagataatattctacttgtgggatcttacattacggaaatagtttgacgtttttgacctcaagagtatgtataatttgaagagatactttgtaactatgcttgggtgatattgagcagttcctaaagaataattcatttaaaaaaaaagaagaaaaaaaaagaagaattcatttaaataacctgatcctttcatttgcccttttcgaatttacagatactacttgtacatttggcataactagttgaaattggccattcgtaccatgaataaatctgatagtttccttgttaggaagagattgtaagtaaatacagtcattgcagtcagaacagtattagtgaaccttgtgtggtgttttcaagctctttaaaatggtacaatgtagcacatttgctttcatttctttttttatttttggcatttgaccttgtattctttctgaagctctatatgtgtttttattagtcaataatctggcaagtagcactttgcctgtgcagtttgctggagtgtagatgtacatatgaggatttcccgggaggtgcacttctttgaagaacttcctaaagtacctgtatagtagttttcatcttaatattcagtatttaatcttcagtttgtgctttgtaaactcatgacttaattggtcagaaactttttagtgtctttataaaattttgtatacatatttatactaaacacattgtgatactgtatttgaatgaatggtgaaaaaatatttgctattggaattatgtgcactgacaagaaatgttataaagagaatgcctttaataaatcttttcagcattagaattgaaaaaaaaaaaaaaaa

SEQ ID NO: 15 - DNA, 2486 bp, Homo sapiens
gagatttgattcccttggcgggcggaagcggccacaacccggcgatcgaaaagattcttaggaacgccgtaccagccgcgtctctcaggacagcaggcccctgtccttctgtcgggcgccgctcagccgtgccctccgcccctcaggttctttttctaattccaaataaacttgcaagaggactatgaaagattatgatgaacttctcaaatattatgaattacatgaaactattgggacaggtggctttgcaaaggtcaaacttgcctgccatatccttactggagagatggtagctataaaaatcatggataaaaacacactagggagtgatttgccccggatcaaaacggagattgaggccttgaagaacctgagacatcagcatatatgtcaactctaccatgtgctagagacagccaacaaaatattcatggttcttgagtactgccctggaggagagctgtttgactatataatttcccaggatcgcctgtcagaagaggagacccgggttgtcttccgtcagatagtatctgctgttgcttatgtgcacagccagggctatgctcacagggacctcaagccagaaaatttgctgtttgatgaatatcataaattaaagctgattgactttggtctctgtgcaaaacccaagggtaacaaggattaccatctacagacatgctgtgggagtctggcttatgcagcacctgagttaatacaaggcaaatcatatcttggatcagaggcagatgtttggagcatgggcatactgttatatgttcttatgtgtggatttctaccatttgatgatgataatgtaatggctttatacaagaagattatgagaggaaaatatgatgttcccaagtggctctctcccagtagcattctgcttcttcaacaaatgctgcaggtggacccaaagaaacggatttctatgaaaaatctattgaaccatccctggatcatgcaagattacaactatcctgttgagtggcaaagcaagaatccttttattcacctcgatgatgattgcgtaacagaactttctgtacatcacagaaacaacaggcaaacaatggaggatttaatttcactgtggcagtatgatcacctcacggctacctatcttctgcttctagccaagaaggctcggggaaaaccagttcgtttaaggctttcttctttctcctgtggacaagccagtgctaccccattcacagacatcaagtcaaataattggagtctggaagatgtgaccgcaagtgataaaaattatgtggcgggattaatagactatgattggtgtgaagatgatttatcaacaggtgctgctactccccgaacatcacagtttaccaagtactggacagaatcaaatggggtggaatctaaatcattaactccagccttatgcagaacacctgcaaataaattaaagaacaaagaaaatgtatatactcctaagtctgctgtaaagaatgaagagtactttatgtttcctgagccaaagactccagttaataagaaccagcataagagagaaatactcactacgccaaatcgttacactacaccctcaaaagctagaaaccagtgcctgaaagaaactccaattaaaataccagtaaattcaacaggaacagacaagttaatgacaggtgtcattagccctgagaggcggtgccgctcagtggaattggatctcaaccaagcacatatggaggagactccaaaaagaaagggagccaaagtgtttgggagccttgaaagggggttggataaggttatcactgtgctcaccaggagcaaaaggaagggttctgccagagacgggcccagaagactaaagcttcactataacgtgactacaactagattagtgaatccagatcaactgttgaatgaaataatgtctattcttccaaagaagcatgttgactttgtacaaaagggttatacactgaagtgtcaaacacagtcagattttgggaaagtgacaatgcaatttgaattagaagtgtgccagcttcaaaaacccgatgtggtgggtatcaggaggcagcggcttaagggcgatgcctgggtttacaaaagattagtggaagacatcctatctagctgcaaggtataattgatggattcttccatcctgccggatgagtgtgggtgtgatacagcctacataaagactgttatgatcgctttgattttaaagttcattggaactaccaacttgtttctaaagagctatcttaagaccaatatctctttgtttttaaacaaaagatattattttgtgtatgaatctaaatcaagcccatctgtcattatgttactgtcttttttaatcatgtggttttgtatattaataattgttgactttcttagattcacttccatatgtgaatgtaagctcttaactatgtctctttgtaatgtgtaatttctttctgaaataaaaccatttgtgaatatag

SEQ ID NO: 16 - DNA, 5101 bp, Homo sapiens
agcgcagccattggtccggctactctgtctctttttcaaattgaggcgccgagtcgttgcttagtttctggggattcgggcggagacgagattagtgatttggcggctccgactggcgcgggacaaacgccacggccagagtaccgggtagagagcggggacgccgacctgcgtgcgtcggtcctccaggccacgccagcgcccgagagggaccagggagactccggcccctgtcggccgccaagcccctccgcccctcacagcgcccaggtccgcggccgggccttgattttttggcggggaccgtcatggcgtcgcagccaaattcgtctgcgaagaagaaagaggagaaggggaagaacatccaggtggtggtgagatgcagaccatttaatttggcagagcggaaagctagcgcccattcaatagtagaatgtgatcctgtacgaaaagaagttagtgtacgaactggaggattggctgacaagagctcaaggaaaacatacacttttgatatggtgtttggagcatctactaaacagattgatgtttaccgaagtgttgtttgtccaattctggatgaagttattatgggctataattgcactatctttgcgtatggccaaactggcactggaaaaacttttacaatggaaggtgaaaggtcacctaatgaagagtatacctgggaagaggatcccttggctggtataattccacgtacccttcatcaaatttttgagaaacttactgataatggtactgaattttcagtcaaagtgtctctgttggagatctataatgaagagctttttgatcttcttaatccatcatctgatgtttctgagagactacagatgtttgatgatccccgtaacaagagaggagtgataattaaaggtttagaagaaattacagtacacaacaaggatgaagtctatcaaattttagaaaagggggcagcaaaaaggacaactgcagctactctgatgaatgcatactctagtcgttcccactcagttttctctgttacaatacatatgaaagaaactacgattgatggagaagagcttgttaaaatcggaaagttgaacttggttgatcttgcaggaagtgaaaacattggccgttctggagctgttgataagagagctcgggaagctggaaatataaatcaatccctgttgactttgggaagggtcattactgcccttgtagaaagaacacctcatgttccttatcgagaatctaaactaactagaatcctccaggattctcttggagggcgtacaagaacatctataattgcaacaatttctcctgcatctctcaatcttgaggaaactctgagtacattggaatatgctcatagagcaaagaacatattgaataagcctgaagtgaatcagaaactcaccaaaaaagctcttattaaggagtatacggaggagatagaacgtttaaaacgagatcttgctgcagcccgtgagaaaaatggagtgtatatttctgaagaaaattttagagtcatgagtggaaaattaactgttcaagaagagcagattgtagaattgattgaaaaaattggtgctgttgaggaggagctgaatagggttacagagttgtttatggataataaaaatgaacttgaccagtgtaaatctgacctgcaaaataaaacacaagaacttgaaaccactcaaaaacatttgcaagaaactaaattacaacttgttaaagaagaatatatcacatcagctttggaaagtactgaggagaaacttcatgatgctgccagcaagctgcttaacacagttgaagaaactacaaaagatgtatctggtctccattccaaactggatcgtaagaaggcagttgaccaacacaatgcagaagctcaggatatttttggcaaaaacctgaatagtctgtttaataatatggaagaattaattaaggatggcagctcaaagcaaaaggccatgctagaagtacataagaccttatttggtaatctgctgtcttccagtgtctctgcattagataccattactacagtagcacttggatctctcacatctattccagaaaatgtgtctactcatgtttctcagatttttaatatgatactaaaagaacaatcattagcagcagaaagtaaaactgtactacaggaattgattaatgtactcaagactgatcttctaagttcactggaaatgattttatccccaactgtggtgtctatactgaaaatcaatagtcaactaaagcatattttcaagacttcattgacagtggccgataagatagaagatcaaaaaaaggaactagatggctttctcagtatactgtgtaacaatctacatgaactacaagaaaataccatttgttccttggttgagtcacaaaagcaatgtggaaacctaactgaagacctgaagacaataaagcagacccattcccaggaactttgcaagttaatgaatctttggacagagagattctgtgctttggaggaaaagtgtgaaaatatacagaaaccacttagtagtgtccaggaaaatatacagcagaaatctaaggatatagtcaacaaaatgacttttcacagtcaaaaattttgtgctgattctgatggcttctcacaggaactcagaaattttaaccaagaaggtacaaaattggttgaagaatctgtgaaacactctgataaactcaatggcaacctggaaaaaatatctcaagagactgaacagagatgtgaatctctgaacacaagaacagtttatttttctgaacagtgggtatcttccttaaatgaaagggaacaggaacttcacaacttattggaggttgtaagccaatgttgtgaggcttcaagttcagacatcactgagaaatcagatggacgtaaggcagctcatgagaaacagcataacatttttcttgatcagatgactattgatgaagataaattgatagcacaaaatctagaacttaatgaaaccataaaaattggtttgactaagcttaattgctttctggaacaggatctgaaactggatatcccaacaggtacgacaccacagaggaaaagttatttatacccatcaacactggtaagaactgaaccacgtgaacatctccttgatcagctgaaaaggaaacagcctgagctgttaatgatgctaaactgttcagaaaacaacaaagaagagacaattccggatgtggatgtagaagaggcagttctggggcagtatactgaagaacctctaagtcaagagccatctgtagatgctggtgtggattgttcatcaattggcggggttccatttttccagcataaaaaatcacatggaaaagacaaagaaaacagaggcattaacacactggagaggtctaaagtggaagaaactacagagcacttggttacaaagagcagattacctctgcgagcccagatcaacctttaattcacttgggggttggcaattttatttttaaagaaaacttaaaaataaaacctgaaaccccagaacttgagccttgtgtatagattttaaaagaatatatatatcagccgggcgcggtggctcatgcctgtaatcccagcactttgggaggctgaggcgggtggattgcttgagcccaggagtttgagaccagcctggccaacgtggcaaaacctcgtctctgttaaaaattagccgggcgtggtggcacactcctgtaatcccagctactggggaggctgaggcacgagaatcacttgaacccaggaagcggggttgcagtgagccaaaggtacaccactacactccagcctgggcaacagagcaagactcggtctcaaaaacaaaatttaaaaaagatataaggcagtactgtaaattcagttgaattttgatatctacccatttttctgtcatccctatagttcactttgtattaaattgggtttcatttgggatttgcaatgtaaatacgtatttctagttttcatataaagtagttcttttataacaaatgaaaagtatttttcttgtatattattaagtaatgaatatataagaactgtactcttctcagcttgagcttacataggtaaatatcaccaacatctgtccttagaaaggaccatctcatgttttttttcttgctatgacttgtgtattttcttgcatcctccctagacttccctatttcgctttctcctcggctcactttctccctttttatttttcaccaaaccatttgtagagctacaaaaggtatcctttcttattttcagtagtcagaattttatctagaaatcttttaacacctttttagtggttatttctaaaatcactgtcaacaataaatctaaccctagttgtatccctcctttcagtatttttcacttgttgccccaaatgtgaaagcatttcattcctttaagaggcctaactcattcaccctgacagagttcacaaaaagcccacttaagagtatacattgctattatgggagaccacccagacatctgactaatggctctgtgcccacactccaagacctgtgccttttagagaagctcacaatgatttaaggactgtttgaaacttccaattatgtctataatttatattcttttgtttacatgatgaaactttttgttgttgcttgtttgtatataatacaatgtgtacatgtatctttttctcgattcaaatcttaacccttaggactctggtatttttgatctggcaaccatatttctggaagttgagatgtttcagcttgaagaaccaaaacagaaggaatatgtacaaagaataaattttctgctcacgatgagtttagtgtgtaaagtttagagacatctgactttgatagctaaattaaaccaaaccctattgaagaattgaatatatgctacttcaagaaactaaattgatctcgtagaattatcttaataaaataatggctataatttctctgcaaaatcagatgtcagcataagcgatggataatacctaataaactgccctcagtaaatccatggttaataaatgtggtttctacattaaaaaaaaaaaaaaaaaaaaaaaaa

SEQ ID NO: 17 - DNA, 1285 bp, Homo sapiens
aattacaaattcccaatgcagttacaggatcctgggaagcagagtgtctggatggaacctgagctgggtctctgactcacttctgactttagttttttcaagggggaacatggcaaaggtgttcagtttcatccttgttaccaccgctctgacaatgggcagggaaatttcggcgctcgaggactgtgcccaggagcagatgcggctcagagcccaggtgcgcctgcttgagacccgggtcaaacagcaacaggtcaagatcaagcagcttttgcaggagaatgaagtccagttccttgataaaggagatgagaatactgtcattgatcttggaagcaagaggcagtatgcagattgttcagagattttcaatgatgggtataagctcagtggattttacaaaatcaaacctctccagagcccagcagaattttctgtttattgtgacatgtccgatggaggaggatggactgtaattcagagacgatctgatggcagtgaaaactttaacagaggatggaaagactatgaaaatggctttggaaattttgtccaaaaacatggtgaatattggctgggcaataaaaatcttcacttcttgaccactcaagaagactacactttaaaaatcgaccttgcagattttgaaaaaaatagccgttatgcacaatataagaatttcaaagttggagatgaaaagaatttctacgagttgaatattggggaatattctggaacagctggagattcccttgcggggaattttcatcctgaggtgcagtggtgggctagtcaccaaagaatgaaattcagcacgtgggacagagatcatgacaactatgaagggaactgcgcagaagaagatcagtctggctggtggtttaacaggtgtcactctgcaaacctgaatggtgtatactacagcggcccctacacggctaaaacagacaatgggattgtctggtacacctggcatgggtggtggtattctctgaaatctgtggttatgaaaattaggccaaatgattttattccaaatgtaatttaattgctgctgttgggctttcgtttctgcaattcagctttgtttaaagtgatttgaaaaatactcattctgaacatatccatgcgcaatcatgataactgttgtgagtagtgcttttcattcttctcacttgcctttgttacttaatgtgctttcagtacagcagatatgcaatattcaccaaataaatgtagactgtgttaaaaaaaaaaaaaaaaaaaaaaaaaaaaaaaaaaaaaa

SEQ ID NO: 18 - DNA, 2127 bp, Homo sapiens
ataatcacgacacattcaacacagatgtgagcgtttggagagagcttcagccttcagcacaggcaactgtgttctattcaccgggctgtgctcctcggaagggctggtggttcctgtaaaagtttgtgctccccgcgcctttccttccttccctcgtctttggcgcctgcgggcaccgggagtcctgggagagggaggaggaacggcgaatcggcggggcgtgtccttcacgccgccccgtagctcggggccgaagctggcgcagcgcgcgactttttgaaagccaggagggttcgaattgcaacggcagctgccgggcgtatgtgttggtgctagaggcagctgcagggtctcgctgggggccgctcgggaccaattttgaagaggtacttggccacgacttattttcacctccgacctttccttccaggcggtgagactctggactgagagtggctttcacaatggaagggatcagtaatttcaagacaccaagcaaattatcagaaaaaaagaaatctgtattatgttcaactccaactataaatatcccggcctctccgtttatgcagaagcttggctttggtactggggtaaatgtgtacctaatgaaaagatctccaagaggtttgtctcattctccttgggctgtaaaaaagattaatcctatatgtaatgatcattatcgaagtgtgtatcaaaagagactaatggatgaagctaagattttgaaaagccttcatcatccaaacattgttggttatcgtgcttttactgaagccaatgatggcagtctgtgtcttgctatggaatatggaggtgaaaagtctctaaatgacttaatagaagaacgatataaagccagccaagatccttttccagcagccataattttaaaagttgctttgaatatggcaagagggttaaagtatctgcaccaagaaaagaaactgcttcatggagacataaagtcttcaaatgttgtaattaaaggcgattttgaaacaattaaaatctgtgatgtaggagtctctctaccactggatgaaaatatgactgtgactgaccctgaggcttgttacattggcacagagccatggaaacccaaagaagctgtggaggagaatggtgttattactgacaaggcagacatatttgcctttggccttactttgtgggaaatgatgactttatcgattccacacattaatctttcaaatgatgatgatgatgaagataaaacttttgatgaaagtgattttgatgatgaagcatactatgcagcgttgggaactaggccacctattaatatggaagaactggatgaatcataccagaaagtaattgaactcttctctgtatgcactaatgaagaccctaaagatcgtccttctgctgcacacattgttgaagctctggaaacagatgtctagtgatcatctcagctgaagtgtggcttgcgtaaataactgtttattccaaaatatttacatagttactatcagtagttattagactctaaaattggcatatttgaggaccatagtttcttgttaacatatggataactatttctaatatgaaatatgcttatattggctataagcacttggaattgtactgggttttctgtaaagttttagaaactagctacataagtactttgatactgctcatgctgacttaaaacactagcagtaaaacgctgtaaactgtaacattaaattgaatgaccattacttttattaatgatctttcttaaatattctatattttaatggatctactgacattagcactttgtacagtacaaaataaagtctacatttgtttaaaacactgaaccttttgctgatgtgtttatcaaatgataactggaagctgaggagaatatgcctcaaaaagagtagctccttggatacttcagactctggttacagattgtcttgatctcttggatctcctcagatctttggtttttgctttaatttattaaatgtattttccatactgagtttaaaatttattaatttgtaccttaagcatttcccagctgtgtaaaaacaataaaactcaaataggatgataaagaataaaggacactttgggtaccagaaaaaaaaaaaa

SEQ ID NO: 19 - DNA, 2339 bp, Homo sapiens
acgcggtgccgcggggcgggagtagaggcggagggaggggacacgggctcattgcggtgtgcgccctgcactctgtccctcactcgccgccgacgacctgtctcgccgagcgcacgccttgccgccgccccgcagaaatgcttcggttacccacagtctttcgccagatgagaccggtgtccagggtactggctcctcatctcactcgggcttatgccaaagatgtaaaatttggtgcagatgcccgagccttaatgcttcaaggtgtagaccttttagccgatgctgtggccgttacaatggggccaaagggaagaacagtgattattgagcagagttggggaagtcccaaagtaacaaaagatggtgtgactgttgcaaagtcaattgacttaaaagataaatacaaaaacattggagctaaacttgttcaagatgttgccaataacacaaatgaagaagctggggatggcactaccactgctactgtactggcacgctctatagccaaggaaggcttcgagaagattagcaaaggtgctaatccagtggaaatcaggagaggtgtgatgttagctgttgatgctgtaattgctgaacttaaaaagcagtctaaacctgtgaccacccctgaagaaattgcacaggttgctacgatttctgcaaacggagacaaagaaattggcaatatcatctctgatgcaatgaaaaaagttggaagaaagggtgtcatcacagtaaaggatggaaaaacactgaatgatgaattagaaattattgaaggcatgaagtttgatcgaggctatatttctccatactttattaatacatcaaaaggtcagaaatgtgaattccaggatgcctatgttctgttgagtgaaaagaaaatttctagtatccagtccattgtacctgctcttgaaattgccaatgctcaccgtaagcctttggtcataatcgctgaagatgttgatggagaagctctaagtacactcgtcttgaataggctaaaggttggtcttcaggttgtggcagtcaaggctccagggtttggtgacaatagaaagaaccagcttaaagatatggctattgctactggtggtgcagtgtttggagaagagggattgaccctgaatcttgaagacgttcagcctcatgacttaggaaaagttggagaggtcattgtgaccaaagacgatgccatgctcttaaaaggaaaaggtgacaaggctcaaattgaaaaacgtattcaagaaatcattgagcagttagatgtcacaactagtgaatatgaaaaggaaaaactgaatgaacggcttgcaaaactttcagatggagtggctgtgctgaaggttggtgggacaagtgatgttgaagtgaatgaaaagaaagacagagttacagatgcccttaatgctacaagagctgctgttgaagaaggcattgttttgggagggggttgtgccctccttcgatgcattccagccttggactcattgactccagctaatgaagatcaaaaaattggtatagaaattattaaaagaacactcaaaattccagcaatgaccattgctaagaatgcaggtgttgaaggatctttgatagttgagaaaattatgcaaagttcctcagaagttggttatgatgctatggctggagattttgtgaatatggtggaaaaaggaatcattgacccaacaaaggttgtgagaactgctttattggatgctgctggtgtggcctctctgttaactacagcagaagttgtagtcacagaaattcctaaagaagagaaggaccctggaatgggtgcaatgggtggaatgggaggtggtatgggaggtggcatgttctaactcctagactagtgctttacctttattaatgaactgtgacaggaagcccaaggcagtgttcctcaccaataacttcagagaagtcagttggagaaaatgaagaaaaaggctggctgaaaatcactataaccatcagttactggtttcagttgacaaaatatataatggtttactgctgtcattgtccatgcctacagataatttattttgtatttttgaataaaaaacatttgtacattcctgatactgggtacaagagccatgtaccagtgtactgctttcaacttaaatcactgaggcatttttactactattctgttaaaatcaggattttagtgcttgccaccaccagatgagaagttaagcagcctttctgtggagagtgagaataattgtgtacaaagtagagaagtatccaattatgtgacaacctttgtgtaataaaaatttgtttaaagttaaaaaaaaaaaaaaaaaaaa

SEQ ID NO: 20 - DNA, 3251 bp, Homo sapiens
gatttggctccgaggaggcggaagtgcagcacagaaagggggtccgtgggggacggtagaagcctggaggaggagcttgagtccagccactgtctgggtactgccagccatcgggcccaggtctctggggttgtcttaccgcagtgagtaccacgcggtactacagagaccggctgcccgtgtgcccggcaggtggagccgcccgcatcagcggcctcggggaatggaagcggagaacgcgggcagctattcccttcagcaagctcaagctttttatacgtttccatttcaacaactgatggctgaagctcctaatatggcagttgtgaatgaacagcaaatgccagaagaagttccagccccagctcctgctcaggaaccagtgcaagaggctccaaaaggaagaaaaagaaaacccagaacaacagaaccaaaacaaccagtggaacccaaaaaacctgttgagtcaaaaaaatctggcaagtctgcaaaatcaaaagaaaaacaagaaaaaattacagacacatttaaagtaaaaagaaaagtagaccgttttaatggtgtttcagaagctgaacttctgaccaagactctccccgatattttgaccttcaatctggacattgtcattattggcataaacccgggactaatggctgcttacaaagggcatcattaccctggacctggaaaccatttttggaagtgtttgtttatgtcagggctcagtgaggtccagctgaaccatatggatgatcacactctaccagggaagtatggtattggatttaccaacatggtggaaaggaccacgcccggcagcaaagatctctccagtaaagaatttcgtgaaggaggacgtattctagtacagaaattacagaaatatcagccacgaatagcagtgtttaatggaaaatgtatttatgaaatttttagtaaagaagtttttggagtaaaggttaagaacttggaatttgggcttcagccccataagattccagacacagaaactctctgctatgttatgccatcatccagtgcaagatgtgctcagtttcctcgagcccaagacaaagttcattactacataaaactgaaggacttaagagatcagttgaaaggcattgaacgaaatatggacgttcaagaggtgcaatatacatttgacctacagcttgcccaagaggatgcaaagaagatggctgttaaggaagaaaaatatgatccaggttatgaggcagcatatggtggtgcttacggagaaaatccatgcagcagtgaaccttgtggcttctcttcaaatgggctaattgagagcgtggagttaagaggagaatcagctttcagtggcattcctaatgggcagtggatgacccagtcatttacagaccaaattccttcctttagtaatcactgtggaacacaagaacaggaagaagaaagccatgcttaagaatggtgcttctcagctctgcttaaatgctgcagttttaatgcagttgtcaacaagtagaacctcagtttgctaactgaagtgttttattagtattttactctagtggtgtaattgtaatgtagaacagttgtgtggtagtgtgaaccgtatgaacctaagtagtttggaagaaaaagtagggtttttgtatactagcttttgtatttgaattaattatcattccagctttttatatactatatttcatttatgaagaaattgattttcttttgggagtcacttttaatctgtaattttaaaatacaagtctgaatatttatagttgattcttaactgcataaacctagatataccattatcccttttatacctaagaagggcatgctaataattaccactgtcaaagaggcaaaggtgttgatttttgtatatgaagttaagcctcagtggagtctcatttgttagtttttagtggtaactaagggtaaactcagggttccctgagctatatgcacactcagacctctttgctttaccagtggtgtttgtgagttgctcagtagtaaaaactggcccttacctgacagagccctggctttgacctgctcagccctgtgtgttaatcctctagtagccaattaactactctggggtggcaggttccagagaatgcagtagaccttttgccactcatctgtgttttacttgagacatgtaaatatgatagggaaggaactgaatttctccattcatatttataaccattctagttttatcttccttggctttaagagtgtgccatggaaagtgataagaaatgaacttctaggctaagcaaaaagatgctggagatatttgatactctcatttaaactggtgctttatgtacatgagatgtactaaaataagtaatatagaatttttcttgctaggtaaatccagtaagccaataattttaaagattctttatctgcatcattgctgtttgttactataaattaaatgaacctcatggaaaggttgaggtgtatacctttgtgattttctaatgagttttccatggtgctacaaataatccagactaccaggtctggtagatattaaagctgggtactaagaaatgttatttgcatcctctcagttactcctgaatattctgatttcatacgtacccagggagcatgctgttttgtcaatcaatataaaatatttatgaggtctcccccacccccaggaggttatatgattgctcttctctttataataagagaaacaaattcttattgtgaatcttaacatgctttttagctgtggctatgatggattttattttttcctaggtcaagctgtgtaaaagtcatttatgttatttaaatgatgtactgtactgctgtttacatggacgttttgtgcgggtgctttgaagtgccttgcatcagggattaggagcaattaaattattttttcacgggactgtgtaaagcatgtaactaggtattgctttggtatataactattgtagctttacaagagattgttttatttgaatggggaaaataccctttaaattatgacggacatccactagagatgggtttgaggattttccaagcgtgtaataatgatgtttttcctaacatgacagatgagtagtaaatgttgatatatcctatacatgacagtgtgagactttttcattaaataatattgaaagattttaaaattcatttgaaagtctgatggcttttacaataaaagatattaagaattgttatccttaacttaaaaaaaa

SEQ ID NO: 21 - DNA, 3207 bp, Homo sapiens
gattgcgcacccgcgacttcagcccgaggggcggggattttcttggagcgacgggacgcgacgccaatcgcgacgaggcttcgccccgtggcgcggtttgaaattttgcggggctcaacggctcgcggagcggctacgcggagtgacatcgccggtgtttgcgggtggttgttgctctcggggccgtgtggagtaggtctggacctggactcacggctgcttggagcgtccgccatgaggagaagtgaggtgctggcggaggagtccatagtatgtctgcagaaagccctaaatcaccttcgggaaatatgggagctaattgggattccagaggaccagcggttacaaagaactgaggtggtaaagaagcatatcaaggaactcctggatatgatgattgctgaagaggaaagcctgaaggaaagactcatcaaaagcatatccgtctgtcagaaagagctgaacactctgtgcagcgagttacatgttgagccatttcaggaagaaggagagacgaccatcttgcaactagaaaaagatttgcgcacccaagtggaattgatgcgaaaacagaaaaaggagagaaaacaggaactgaagctacttcaagagcaagatcaagaactgtgcgaaattctttgtatgccccactatgatattgacagtgcctcagtgcccagcttagaagagctgaaccagttcaggcaacatgtgacaactttgagggaaacaaaggcttctaggcgtgaggagtttgtcagtataaagagacagatcatactgtgtatggaagaattagaccacaccccagacacaagctttgaaagagatgtggtgtgtgaagacgaagatgccttttgtttgtctttggagaatattgcaacactacaaaagttgctacggcagctggaaatgcagaaatcacaaaatgaagcagtgtgtgaggggctgcgtactcaaatccgagagctctgggacaggttgcaaatacctgaagaagaaagagaagctgtggccaccattatgtctgggtcaaaggccaaggtccggaaagcgctgcaattagaagtggatcggttggaagaactgaaaatgcaaaacatgaagaaagtgattgaggcaattcgagtggagctggttcagtactgggaccagtgcttttatagccaggagcagagacaagcttttgcccctttctgtgctgaggactacacagaaagtctgctccagctccacgatgctgagattgtgcggttaaaaaactactatgaagttcacaaggaactctttgaaggtgtccagaagtgggaagaaacctggaggcttttcttagagtttgagagaaaagcttcagatccaaatcgatttacaaaccgaggaggaaatcttctaaaagaagaaaaacaacgagccaagctccagaaaatgctgcccaagctggaagaagagttgaaggcacgaattgaattgtgggaacaggaacattcaaaggcatttatggtgaatgggcagaaattcatggagtatgtggcagaacaatgggagatgcatcgattggagaaagagagagccaagcaggaaagacaactgaagaacaaaaaacagacagagacagagatgctgtatggcagcgctcctcgaacacctagcaagcggcgaggactggctcccaatacaccgggcaaagcacgtaagctgaacactaccaccatgtccaatgctacggccaatagtagcattcggcctatctttggagggacagtctaccactcccccgtgtctcgacttcctccttctggcagcaagccagtcgctgcttccacctgttcagggaagaaaacaccccgtactggcaggcatggagccaacaaggagaacctggagctcaacggcagcatcctgagtggtgggtaccctggctcggcccccctccagcgcaacttcagcattaattctgttgccagcacctattctgagtttgcgaaggatccgtccctctctgacagttccactgttgggcttcagcgagaactttcaaaggcttccaaatctgatgctacttctggaatcctcaattcaaccaacatccagtcctgagaagccctgatcagtcaaccagctgtggcttcctgtgcctagactggacctaattatatgggggtgactttagtttttcttcagcttaggcgtgcttgaaaccttggccaggttccatgaccatgggcctaacttaaagatgtgaatgagtgttacagttgaaagcccatcataggtttagtggtcctaggagacttggttttgacttatatacatgaaaagtttatggcaagaagtgcaaattttagcatatggggcctgacttctctaccacataattctacttgctgaagcatgatcaaagcttgttttatttcaccactgtaggaaaatgattgactatgcccatccctgggggtaattttggcatgtatacctgtaactagtaattaacatcttttttgtttaggcatgttcaattaatgctgtagctatcatagctttgctcttacctgaagccttgtccccaccacacaggacagccttcctcctgaagagaatgtctttgtgtgtccgaagttgagatggcctgccctactgccaaagaggtgacaggaaggctgggagcagctttgttaaattgtgttcagttctgttacacagtgcattgccctttgttgggggtatgcatgtatgaacacacatgcttgtcggaacgctttctcggcgtttgtcccttggctctcatctcccccattcctgtgcctactttgcctgagttcttctacccccgcagttgccagccacattgggagtctgtttgttccaatgggttgagctgtctttgtcgtggagatctggaactttgcacatgtcactactggggaggtgttcctgctctagcttccacgatgaggcgccctctttacctatcctctcaatcactactcttcttgaagcactattatttattcttccgctgtctgcctgcagcagtactactgtcaacatagtgtaaatggttctcaaaagcttaccagtgtggacttggtgttagccacgctgtttactcatacagtacgtgtcctgtttttaaaatatacaattattcttaaaaataaattaaaatctgtatacttacatttcaaaaagaaaaaaaaaaaaaaaaaa

SEQ ID NO: 22 - DNA, 5625 bp, Homo sapiens
acctagcgccccctcccccgggagcgcggaggagcattaataaacctctaagccgaggagaaaactctggctggggcagtgcgctgagcgccggaggagcgtaggcagggcagcgctggcgccagtggcgacaggagccgcgcgaccggcaaaaatacacgggaggccgtcgccgaaaagagtccgcggtcctctctcgtaaacacactctcctccaccggcgcctccccctccgctctgcgcgccgcccggctgggcgcccgaggccgctccgactgctatgtgaccgcgaggctgcgggaggaaggggacagggaagaagaggctctcccgcgggagcccttgaggaccaagtttgcggccacttctgcaggcgtcccttcttagctctcgcccgcccctttctgcagcctaggcggcccgggttctcttctcttcctcgcgcgcccagccgcctcggttcccggcgaccatggtgacgatggaggagctgcgggagatggactgcagtgtgctcaaaaggctgatgaaccgggacgagaatggcggcggcgcgggcggcagcggcagccacggcaccctggggctgccgagcggcggcaagtgcctgctgctggactgcagaccgttcctggcgcacagcgcgggctacatcctaggttcggtcaacgtgcgctgtaacaccatcgtgcggcggcgggctaagggctccgtgagcctggagcagatcctgcccgccgaggaggaggtacgcgcccgcttgcgctccggcctctactcggcggtcatcgtctacgacgagcgcagcccgcgcgccgagagcctccgcgaggacagcaccgtgtcgctggtggtgcaggcgctgcgccgcaacgccgagcgcaccgacatctgcctgctcaaaggcggctatgagaggttttcctccgagtacccagaattctgttctaaaaccaaggccctggcagccatcccacccccggttccccccagtgccacagagcccttggacctgggctgcagctcctgtgggaccccactacacgaccaggggggtcctgtggagatccttcccttcctctacctcggcagtgcctaccatgctgcccggagagacatgctggacgccctgggcatcacggctctgttgaatgtctcctcggactgcccaaaccactttgaaggacactatcagtacaagtgcatcccagtggaagataaccacaaggccgacatcagctcctggttcatggaagccatagagtacatcgatgccgtgaaggactgccgtgggcgcgtgctggtgcactgccaggcgggcatctcgcggtcggccaccatctgcctggcctacctgatgatgaagaaacgggtgaggctggaggaggccttcgagttcgttaagcagcgccgcagcatcatctcgcccaacttcagcttcatggggcagctgctgcagttcgagtcccaggtgctggccacgtcctgtgctgcggaggctgctagcccctcgggacccctgcgggagcggggcaagacccccgccacccccacctcgcagttcgtcttcagctttccggtctccgtgggcgtgcactcggcccccagcagcctgccctacctgcacagccccatcaccacctctcccagctgttagagccgccctgggggccccagaaccagagctggctcccagcaagggtaggacgggccgcatgcgggcagaaagttgggactgagcagctgggagcaggcgaccgagctccttccccatcatttctccttggccaacgacgaggccagccagaatggcaataaggactccgaatacataataaaagcaaacagaacactccaacttagagcaataacggctgccgcagcagccagggaagaccttggtttggtttatgtgtcagtttcacttttccgatagaaatttcttacctcatttttttaagcagtaaggcttgaagtgatgaaacccacagatcctagcaaatgtgcccaaccagctttactaaagggggaggaagggagggcaaagggatgagaagacaagtttcccagaagtgcctggttctgtgtacttgtccctttgttgtcgttgttgtagttaaaggaatttcattttttaaaagaaatcttcgaaggtgtggttttcatttctcagtcaccaacagatgaataattatgcttaataataaagtatttattaagactttcttcagagtatgaaagtacaaaaagtctagttacagtggatttagaatatatttatgttgatgtcaaacagctgagcaccgtagcatgcagatgtcaaggcagttaggaagtaaatggtgtcttgtagatatgtgcaaggtagcatgatgagcaacttgagtttgttgccactgagaagcaggcgggttgggtgggaggaggaagaaagggaagaattaggtttgaattgctttttaaaaaaaaaagaaaagaaaaagacagcatctcactatgttgccaaggctcatcttgagaagcaggcgggttgggtgggaggaggaagaaagggaagaattaggtttgaattgcttttttaaaaaaaaagaaaagaaaaaaaaagacagcatctcactatgttgccaaggctcatctcaagctcttgggctcaagagatcctcccacctcggcctcctgagtagctgggactgcaggtgtgtgtcatcatgaccaatgtgaattgcttttgaagctggttcatgggcatgtaggccaccgaagcaattttagaccacagtaagtcaagcttttttccctccgatgatcactgggtggttgcagcattttttgcataaacctgcctaagacttgtctatcgtctgtgatcaatatgccatattacactaaggtgctcctggaaaattgggtgcagttcaaattttcctacagcaaatcatttggcaaggccagccattggggaaaccagacaactagagataaccctgaaatgaatccttttgtaaattgaagcaccatcttttctttttttgcataaattggaggttttaattttagggcagttacctgaagtgaaatataccaacaatttcttgtgttctttaaattcctagttaggtgaatatttttgaaggtcctcttttgaataaagaggggaatggacaccacatttcaggtcttctcgaagtgtggaagggcaagagagcatcagtgagctgatggtggattgcttacatcggattccattggtatgaatttcccaaactggaaatcaaagcgccagggtggggttggggctgactgctggtgagggggctggccgctggctcccgtgacgtgcgtcatgggcacgcaggcgccattttgaatctatcgtcggcacgtgggtgccattttgaatccttagttgggcctttctaaatggagaatggctttggagggagacacgttttctgtggggagggtttgggggggagggaggagggaacaagctacatgctattttgtttgtagtattgtggaacagtcttgttatggagtgccagcttagaggttgttgcaaacttgtctagaagtgagagcatggttttttttagccctttgagagtctacatctaatgaacattcttgctcacccataaataacgtcaagcctcaatgtcaccgtcacgttgggatactctttctcatctggcatcctagacaggacaaggttggttacctttccttccatgaaccatgaacctgtgacggcatcattcatcctgacttcaccaagctccgcctgtgggtgaggccagagctcccactggcaatttttagaagagccagaggctccctgcttcctctagaaataacagttcagggtgaagcatggagggtttcagttcccagacaatggaaccatttagagacaacacagttggacatttccactttttccttgattcctggaagtccagtgggttctgcagctgaaaaagccctgggtcccagcagcagagagacaggacagaggggatgcttgggcggggagggacggtaacctgcagaacagattccatttttatagaacgagtacacgtttgctaaaacagtcctgctttcccagactggattcccaccacagggacagtcggaactcaggactagctccagcgacatctttcctccgaattcaagccttctatcacaatgtcaaaacagctatttataaagccattttcattgtacttgataacagcacgagtcccaaaacttttagaaataaaataggacattggcttgattgaaaagagggactttttaaaaattgttctttcgtcagaagccttttggatgacttacaatagctctgatgaagataccaccccagcgtcagtccaataggtcagtgagtttcaacaggcatccatccctcccatgaagggattctggtgatgggaagtttctgtaatgacaggaaagcattgaccctcattgattgtcaactttggtattagccatgaaagacaggatgctcattgggtgttctgtagagtgaggaatgctgcctattccctcccagaacgtctgacccaggggtgtgtgttgaggagccctgggggaaatggaccaagttttcccacagagcagtattaggctgaagagcaggtgactggtaggccccagctcccatcattccctcccaaagccattttgttcagttgctcatccacgctggattccagagagttttccaatttgggaagccatgagaaaggtttttaaatcttgggaagatggagagagggacataggatagttgactccaacatgacaggaagaggctggagattgggaattggccatcaaccaagcctgtagtagtaaagccatggtcccgcattggaattacttggggaacttatacagttctgatacccaggctctcctagaccagttcaaccaattctaggtgggggactcaggcatcagtgtgtttcgtagctccccgggtgttttccctgtgcagccgagcttgggaaactgccatgctttttggatgtcaaggcgctgttggaggctgggtgtgacagcacagagccaggttgtcttgtggaaaccacagccacgggtttgccactggctcagcatggcctcactgccagtcccagcctggctgagggacaagatggtttctcttgggagttcctgagtggagcacccttccaggctttttgaaagccagctgatctgtggagccttgttaagggactcaatacggtgtttggatattgatgtttttccttgagactgtcttgtccatcaataaagatggaggatgtctcctctttgaaccccgcttccccaccagtactctctctcccttagagtttatgagttattcaaggaggagacttcttaaagacagcaacgcaattcttgtaacttgtgtaaatagccccatctttcagagtgataccatttctacatttgataatgcctgtattcctgtaggatgtatatagtttaggggattttttttttgtttggttttgttttttagaagtcaatatgtctggttttatttattgcttgaaaaagatcatttgaaaaaaataaatacattttcaaccactaaaa

SEQ ID NO: 23 - DNA, 2537 bp, Homo sapiens
ggaagtcccacctgcgcccgacggcggaagttccgggagtgccaagtacccgcgtgcatacggctgccggcatggcacattacaacttcaagaaaattacggtggtgccgtccgccaaggacttcatagacctcacgttgtcgaagactcaacgaaagactccaaccgttattcataaacattaccaaatacatcgcattagacatttttacatgagaaaagtcaaatttactcaacagaattaccatgatagactttcacaaattctaacagatttccccaaattggatgatattcatccgttctatgctgatttgatgaatattctctacgacaaggatcattacaagttggctctggggcaaataaatattgccaaaaatttagtggacaatgttgctaaagattatgtgcgactgatgaagtatggcgactctctctaccgctgcaaacagctgaagcgtgcggccctgggacggatgtgcacagtgatcaagaggcagaagcagagtttggagtatttggagcaagtgcgtcagcatttatcccgtttgccaaccattgatccgaataccaggaccctgcttttgtgtgggtacccaaatgttgggaagtccagcttcatcaacaaggtgacgagagcagacgtggatgtccagccctatgcgttcacaaccaagtctctgtttgttgggcacatggattataagtatctacgttggcaggttgtagacactcctgggatcctggaccaccctctggaggataggaacaccatcgagatgcaggccatcactgccctggcccacctccgtgctgcggtcctgtatgtgatggatttgtctgagcagtgtgggcatgggctgagggagcagctagaactcttccagaacatcagacctctcttcatcaacaagcctctcatagttgtagccaacaaatgtgatgtgaagagaatagctgaactttctgaagatgatcagaaaatatttacagatttgcagtctgaaggattccctgtaatagagaccagcaccctgactgaggaaggtgttattaaagttaaaacagaggcttgcgataggcttttggctcatcgagtggaaaccaaaatgaagggaaataaagtgaatgaggtgctgaatagactgcacctggctatcccaaccaggagggacgataaggagaggccccctttcatccctgaaggagtggtggctcgcaggaagaggatggaaactgaggagtccaggaagaagagggaacgagatcttgagctggaaatgggagatgattatattttggatcttcagaagtactgggatttaatgaatttgtctgaaaaacatgataagataccagaaatctgggaaggccataatatagctgattatattgatccagccatcatgaagaaattggaagaattagaaaaagaagaagagctgagaacagctgctggagagtatgacagtgtatctgagagtgaagacgaagagatgctggaaatccgacagctggcaaagcaaattcgagagaaaaagaagttgaaaattctggagtccaaagaaaagaatacacagggacccaggatgccgcgaactgctaagaaggttcagaggacagttttggagaaggagatgcgtagtcttggtgttgacatggacgataaagacgatgcccattacgcagtccaggcaagaagatcccggagcatcactaggaaaagaaagcgggaagactctgctcccccgtcctctgtggcccggagtgggagttgctctcgaactccacgtgacgtttctggtcttagggatgtcaagatggtgaagaaagccaagactatgatgaagaatgctcagaagaagatgaatcggttggggaagaaaggggaggcggatagacacgtgtttgatatgaagcccaagcacttgctgtctgggaagaggaaagctggtaaaaaggacaggagatagtatccgtttggttggcgtggcttcgctagagtgttgctgtttatttcctggtttggcacagtatggtttcatgaaattggagctctgtataaactgaaaaagacaaaataagtaaagcacttgttgctttgctgaaaactatggttaaccctatataggtgtgggaaatttttgtcactgcataatattacaaatattttgagtagacagtgtttccacatttaatggagtatcagttgcttcagattttcagaactgggaagatttactggtgtaactgggttgtttttgatggagaaaaaccttattttcttttgtaagagctgggagcaaacacgtttatgagtgtgtcggaatcccgtgcttaaaatacgctcttaaattattttctagtcttattttacaatgtctcattgtagtctgtcttcaactattttatccaaaataaacctccagaagaaagtagttttcatttacttagctcatgttttggtttagttatagtcgctatggatttggccaaataaaaaggcaaacaacaaaaaaaaaaaaaaaaaaaaa

SEQ ID NO: 24 - DNA, 1687 bp, Homo sapiens
gattgtgggaaggcagctgaactcggcgcctggaaagatggaggcagcggagacagaggcggaagctgcagccctagaggtcctggctgaggtggcaggcatcttggaacctgtaggcctgcaggaggaggcagaactgccagccaagatcctggttgagtttgtggtggactctcagaagaaagacaagctgctctgcagccagcttcaggtagcggatttcctgcagaacatcctggctcaggaggacactgctaagggtctcgaccccttggcttctgaagacacgagccgacagaaggcaattgcagctaaggaacaatggaaagagctgaaggccacctacagggagcacgtagaggccatcaaaattggcctcaccaaggccctgactcagatggaggaagcccagaggaaacggacacaactccgggaagcctttgagcagctccaggccaagaaacaaatggccatggagaaacgcagagcagtccagaaccagtggcagctacaacaggagaagcatctgcagcatctggcggaggtttctgcagaggtgagggagcgtaagacagggactcagcaggagcttgacagggtgtttcagaaacttggaaacctgaagcagcaggcagaacaggagcgggacaagctgcagaggtatcagaccttcctccagcttctgtataccctgcagggtaagctgttgttccctgaggctgaggctgaggcagagaatcttccagatgataaaccccagcagccgactcgaccccaggagcagagtacaggagacaccatggggagagaccctggtgtgtccttcaaggctgttggtctacaacctgctggagatgtaaatttgccatgacttcctggaggacagcagcatggagaaagatcctagaaaaggcctctgacttccctcacctcccaaccatcattacaggaaagactgtgaactcctgagttcagcttgatttctgactacatcccagcaagctctggcatctgtggattaaaatccctggatctctctcagttgtgtatttgttcatcttcatatgctggcaggaacaactattaatacagatactcagaagccaataacatgacaggagctgggactggtttgaacacagggtgtgcagatggggagggggtactggccttgggcctcctatgatgcagacatggtgaatttaattcaaggaggaggagaatgttttaggcaggtggttatatgtgggaagataattttattcatggatccaaatgtttgttgagtcctttctttgtgctaaggttcttgcggtgaaccagaattataacagtgagctcatctgactgttttaggatgtacagcctagtgttaacattcttggtatctttttgtgccttatctaaaacatttctcgatcactggtttcagatgttcatttattatattcttttcaaagattcagagattggcttttgtcatccactattgtatgttttgtttcattgacctctagtgataccttgatctttcccactttctgttttcggattggagaagatgtaccttttttgtcaactcttacttttatcagatgatcaactcacgtatttggatctttatttgttttctcaaataaatatttaaggttatacatttaaaaaaaaaaaaaaaaaaaaaaaaaaa

SEQ ID NO: 25 - DNA, 3849 bp, Homo sapiens
cccttttgcttacttcctagtcccgggcaggccggctcggaggcagcgagaaagcgcagccaggcggctgctcggcgttctctcaggtgactgctcggagttctcccagtgtttggtgttgcaagcaggatccaaaggagacctatagtgactcccaggagctcttagtgaccaagtgaaggcaggaagacagggttctgatggtgtgagtctccttcaactcagcaaaccaccttggtctgcctcgagtttccaacacccctcctgcctgctagtgataggtgtgaggcaggttgatgaacatggaacttttttcttttggtcccaaagcatgctactcctggagtttcattcagtgaatgagaactatagtttggttctgtgagatctctatgaatcaaggcggccactgaagcggagaaaagaaatgcttaaatgttaagaaagtttgaagtgcagaaaaaggttgaagcactggacaatgccacatactttgtggatggtgtgggtcttgggggtcatcatcagcctctccaaggaagaatcctccaatcaggcttctctgtcttgtgaccgcaatggtatctgcaagggcagctcaggatctttaaactccattccctcagggctcacagaagctgtaaaaagccttgacctgtccaacaacaggatcacctacattagcaacagtgacctacagaggtgtgtgaacctccaggctctggtgctgacatccaatggaattaacacaatagaggaagattctttttcttccctgggcagtcttgaacatttagacttatcctataattacttatctaatttatcgtcttcctggttcaagcccctttcttctttaacattcttaaacttactgggaaatccttacaaaaccctaggggaaacatctcttttttctcatctcacaaaattgcaaatcctgagagtgggaaatatggacaccttcactaagattcaaagaaaagattttgctggacttaccttccttgaggaacttgagattgatgcttcagatctacagagctatgagccaaaaagtttgaagtcaattcagaatgtaagtcatctgatccttcatatgaagcagcatattttactgctggagatttttgtagatgttacaagttccgtggaatgtttggaactgcgagatactgatttggacactttccatttttcagaactatccactggtgaaacaaattcattgattaaaaagtttacatttagaaatgtgaaaatcaccgatgaaagtttgtttcaggttatgaaacttttgaatcagatttctggattgttagaattagagtttgatgactgtacccttaatggagttggtaattttagagcatctgataatgacagagttatagatccaggtaaagtggaaacgttaacaatccggaggctgcatattccaaggttttacttattttatgatctgagcactttatattcacttacagaaagagttaaaagaatcacagtagaaaacagtaaagtttttctggttccttgtttactttcacaacatttaaaatcattagaatacttggatctcagtgaaaatttgatggttgaagaatacttgaaaaattcagcctgtgaggatgcctggccctctctacaaactttaattttaaggcaaaatcatttggcatcattggaaaaaaccggagagactttgctcactctgaaaaacttgactaacattgatatcagtaagaatagttttcattctatgcctgaaacttgtcagtggccagaaaagatgaaatatttgaacttatccagcacacgaatacacagtgtaacaggctgcattcccaagacactggaaattttagatgttagcaacaacaatctcaatttattttctttgaatttgccgcaactcaaagaactttatatttccagaaataagttgatgactctaccagatgcctccctcttacccatgttactagtattgaaaatcagtaggaatgcaataactacgttttctaaggagcaacttgactcatttcacacactgaagactttggaagctggtggcaataacttcatttgctcctgtgaattcctctccttcactcaggagcagcaagcactggccaaagtcttgattgattggccagcaaattacctgtgtgactctccatcccatgtgcgtggccagcaggttcaggatgtccgcctctcggtgtcggaatgtcacaggacagcactggtgtctggcatgtgctgtgctctgttcctgctgatcctgctcacgggggtcctgtgccaccgtttccatggcctgtggtatatgaaaatgatgtgggcctggctccaggccaaaaggaagcccaggaaagctcccagcaggaacatctgctatgatgcatttgtttcttacagtgagcgggatgcctactgggtggagaaccttatggtccaggagctggagaacttcaatccccccttcaagttgtgtcttcataagcgggacttcattcctggcaagtggatcattgacaatatcattgactccattgaaaagagccacaaaactgtctttgtgctttctgaaaactttgtgaagagtgagtggtgcaagtatgaactggacttctcccatttccgtctttttgatgagaacaatgatgctgccattctcattcttctggagcccattgagaaaaaagccattccccagcgcttctgcaagctgcggaagataatgaacaccaagacctacctggagtggcccatggacgaggctcagcgggaaggattttgggtaaatctgagagctgcgataaagtcctaggttcccatatttaagaccagtctttgtctagttgggatctttatgtcactagttatagttaagttcattcagacataattatataaaaactacgtggatgtaccgtcatttgaggacttgcttactaaaactacaaaacttcaaattttgtctggggtgctgttttataaacatatgccagatttaaaaattggtttttggtttttcttttttctatgagataaccatgatcataagtctattactgatatctgaatatagtcccttggtatccaagggaattggttgcaggatcctcgtggatatcaaaattcatagatgatcaagtcccttataagagtggcatagtatttgcatataacctgtgtacattctcctgtatactttaaatcatctctagattacttatgatacccaatacaatgtaaatactatgtaaatagttgtactgtctttttatttatattattattgttattttttattttcaaaatttttaaaacatacttttgatccacagttggttgacttcatggatgcagaacccatggatatagagggccaactgtaatctgtagcaactggcttagttcattaggaaacagcacaaatgaacttaagattctcaatgactgtgtcattctttcttcctgctaagagactcctctgtggccacaaaaggcattctctgtcctacctagctgtcacttctctgtgcagctgatctcaagagcaacaaggcaaagtatttggggcactccccaaaacttgttgctattcctagaaaaaagtgctgtgtatttcctattaaactttacaggatgagaaatactagagggtgtattttcatgtgatctggatctgtctttctggctattagataggtttctccagccatagttacttgagagagtgagtacacagtgcaaggtacagtaaattatgtatttctgtaatttaacaaaataaatactcagtttaggagaatttgaaagacaaaaaaaaaaaaaaaa

SEQ ID NO: 26 - DNA, 1698 bp, Homo sapiens
ctgcctggggagcccccccgccccacatcctgccccgcaaaaggcagcttcaccaaagtggggtatttccagcctttgtagctttcacttccacatctaccaagtgggcggagtggccttctgtggacgaatcagattcctctccagcaccgactttaagaggcgagccggggggtcagggtcccagatgcacaggaggagaagcaggagctgtcgggaagatcagaagccagtcatggatgaccagcgcgaccttatctccaacaatgagcaactgcccatgctgggccggcgccctggggccccggagagcaagtgcagccgcggagccctgtacacaggcttttccatcctggtgactctgctcctcgctggccaggccaccaccgcctacttcctgtaccagcagcagggccggctggacaaactgacagtcacctcccagaacctgcagctggagaacctgcgcatgaagcttcccaagcctcccaagcctgtgagcaagatgcgcatggccaccccgctgctgatgcaggcgctgcccatgggagccctgccccaggggcccatgcagaatgccaccaagtatggcaacatgacagaggaccatgtgatgcacctgctccagaatgctgaccccctgaaggtgtacccgccactgaaggggagcttcccggagaacctgagacaccttaagaacaccatggagaccatagactggaaggtctttgagagctggatgcaccattggctcctgtttgaaatgagcaggcactccttggagcaaaagcccactgacgctccaccgaaagtactgaccaagtgccaggaagaggtcagccacatccctgctgtccacccgggttcattcaggcccaagtgcgacgagaacggcaactatctgccactccagtgctatgggagcatcggctactgctggtgtgtcttccccaacggcacggaggtccccaacaccagaagccgcgggcaccataactgcagtgagtcactggaactggaggacccgtcttctgggctgggtgtgaccaagcaggatctgggcccagtccccatgtgagagcagcagaggcggtcttcaacatcctgccagccccacacagctacagctttcttgctcccttcagcccccagcccctcccccatctcccaccctgtacctcatcccatgagaccctggtgcctggctctttcgtcacccttggacaagacaaaccaagtcggaacagcagataacaatgcagcaaggccctgctgcccaatctccatctgtcaacaggggcgtgaggtcccaggaagtggccaaaagctagacagatccccgttcctgacatcacagcagcctccaacacaaggctccaagacctaggctcatggacgagatgggaaggcacagggagaagggataaccctacacccagaccccaggctggacatgctgactgtcctctcccctccagcctttggccttggcttttctagcctatttacctgcaggctgagccactctcttccctttccccagcatcactccccaaggaagagccaatgttttccacccataatcctttctgccgacccctagttccctctgctcagccaagcttgttatcagctttcagggccatggttcacattagaataaaaggtagtaattagaacaaaaaaaaaaaaaaaaaa

SEQ ID NO: 27 - DNA, 1501 bp, Homo sapiens
agcgagtccttcttttcctgactgcagctcttttcattttgccatccttctccagctccatgatggttctgcaggtttctgcggccccccggacagtggctctgacggcgttactgatggtgctgctcacatctgtggtccagggcagggccactccagagaattacgtgtaccagggacggcaggaatgctacgcgtttaatgggacacagcgcttcctggagagatacatctacaaccgggaggagtacgcgcgcttcgacagcgacgtgggggagttccgggcggtgacggagctggggcggcctgctgcggagtactggaacagccagaaggacatcctggaggagaagcgggcagtgccggacagggtatgcagacacaactacgagctggacgaggccgtgaccctgcagcgccgagtccagcctaaggtgaacgtttccccctccaagaaggggcccctgcagcaccacaacctgcttgtctgccacgtgacagatttctacccaggcagcattcaagtccgatggttcctgaatggacaggaggaaacagctggggtcgtgtccaccaacctgatccgtaatggagactggaccttccagatcctggtgatgctggaaatgaccccccagcagggagacgtctacatctgccaagtggagcacaccagcctggacagtcctgtcaccgtggagtggaaggcacagtctgattctgcccagagtaagacattgacgggagctgggggcttcgtgctggggctcatcatctgtggagtgggcatcttcatgcacaggaggagcaagaaagttcaacgaggatctgcataaacagggttcctgacctcaccgaaaagactaatgtgccttagaacaagcatttgctgtgttttgttaacacctggttccaggacagaccctcagcttcccaagaggatactgctgccaagaagttgctctgaagtcagtttctatcgttctgctctttgattcaaagcactgtttctctcactgggcctccaaccatgttcccttcttcttagcaccacaaataatcaaaacccaacataagtgtttgctttcctttaaaaatatgcatcaaatcgtctctcattacttttctctgagggttttagtaaacagtaggagttaataaagaagttcattttggtttacacgtaggaaagaagagaagcatcaaagtggagatatgttaactattgtataatgtggcctgttatacatgacactcttctgaattgactgtatttcagtgagctgcccccaaatcaagtttagtgccctcatccatttatgtctcagaccgctattcttaactattcaatggtgagcagactgcaaatctgcctgataggacccatattcccacagcactaattcaacatatatcttactgagagcatgttttatcattaccattaagaagttaaatgaacatcagaatttaaaatcataaatataatctaatacacttt

SEQ ID NO: 28 - DNA, 1697 bp, Homo sapiens
atttccagtgctagaggcccacagtttcagtctcatctgcctccactcggcctcagttcctcatcactgttcctgtgctcacagtcatcaattatagaccccacaacatgcgccctgaagacagaatgttccatatcagagctgtgatcttgagagccctctccttggctttcctgctgagtctccgaggagctggggccatcaaggcggaccatgtgtcaacttatgccgcgtttgtacagacgcatagaccaacaggggagtttatgtttgaatttgatgaagatgagatgttctatgtggatctggacaagaaggagaccgtctggcatctggaggagtttggccaagccttttcctttgaggctcagggcgggctggctaacattgctatattgaacaacaacttgaataccttgatccagcgttccaaccacactcaggccaccaacgatccccctgaggtgaccgtgtttcccaaggagcctgtggagctgggccagcccaacaccctcatctgccacattgacaagttcttcccaccagtgctcaacgtcacgtggctgtgcaacggggagctggtcactgagggtgtcgctgagagcctcttcctgcccagaacagattacagcttccacaagttccattacctgacctttgtgccctcagcagaggacttctatgactgcagggtggagcactggggcttggaccagccgctcctcaagcactgggaggcccaagagccaatccagatgcctgagacaacggagactgtgctctgtgccctgggcctggtgctgggcctagtcggcatcatcgtgggcaccgtcctcatcataaagtctctgcgttctggccatgacccccgggcccaggggaccctgtgaaatactgtaaaggtgacaaaatatctgaacagaagaggacttaggagagatctgaactccagctgccctacaaactccatctcagcttttcttctcacttcatgtgaaaactactccagtggctgactgaattgctgacccttcaagctctgtccttatccattacctcaaagcagtcattccttagtaaagtttccaacaaatagaaattaatgacactttggtagcactaatatggagattatcctttcattgagccttttatcctctgttctcctttgaagaacccctcactgtcaccttcccgagaataccctaagaccaataaatacttcagtatttcagagcggggagactctgagtcattcttactggaagtctaggaccaggtcacatgtgaatactatttcttgaaggtgtggtttcaacctctgttgccgatgtggttactaaaggttctgatcccacttgaacggaaaggtctgaggatattgattcagtcctgggtttttccctaactacaggatagggtggggtagagaaaggatatttgggggaaattttacttggatgaagattttcttggatgtagtttgaagactgcagtgtttgaagtctctgagggaagagatttggtctgtctggatcaagatttcaggcagattaggattccattcacagcccctgagcttccttcccaaggctgtattgtaattatagcaatatttcatggaggatttttctacatgataaactaagagccaagaaataaaatttttaaaatgccctaaaaaaaaaaaaaaaaaa

SEQ ID NO: 29 - DNA, 1312 bp, Homo sapiens
ttttaatggtcagactctattacaccccacattctcttttcttttattcttgtctgttctgcctcactcccgagctctactgactcccaacagagcgcccaagaagaaaatggccataagtggagtccctgtgctaggatttttcatcatagctgtgctgatgagcgctcaggaatcatgggctatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaatgtacctccagaggtaactgtgctcacaaacagccctgtggaactgagagagcccaacgtcctcatctgtttcatagacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctccaagccctctcccagagactacagagaacgtggtgtgtgccctgggcctgactgtgggtctggtgggcatcattattgggaccatcttcatcatcaagggattgcgcaaaagcaatgcagcagaacgcagggggcctctgtaaggcacatggaggtgatggtgtttcttagagagaagatcactgaagaaacttctgctttaatggctttacaaagctggcaatattacaatccttgacctcagtgaaagcagtcatcttcagcattttccagccctatagccaccccaagtgtggatatgcctcttcgattgctccgtactctaacatctagctggcttccctgtctattgccttttcctgtatctattttcctctatttcctatcattttattatcaccatgcaatgcctctggaataaaacatacaggagtctgtctctgctatggaatgccccatggggcatctcttgtgtacttattgtttaaggtttcctcaaactgtgatttttctgaacacaataaactattttgatgatcttgggtggaaaaaaaaaaaaaaaaaaaaaaaaaaaaaaaaaa

SEQ ID NO: 30 - DNA, 2973 bp, Homo sapiens
gggccgctctctgacatcagagctgctgtagagcggagaggggcaggggtgaagggccacggtggtgcaacccaccacttcctccaaggaggagctgagaggaacaggaagtgtcaggactttacgacccgcgcctccagctgaggtttctagacgtgacccagggcagactggtagcaaagcccccacgcccagccaggagcaccgccgaggactccagcacaccgagggacatgctgggcctgcgccccccactgctcgccctggtggggctgctctccctcgggtgcgtcctctctcaggagtgcacgaagttcaaggtcagcagctgccgggaatgcatcgagtcggggcccggctgcacctggtgccagaagctgaacttcacagggccgggggatcctgactccattcgctgcgacacccggccacagctgctcatgaggggctgtgcggctgacgacatcatggaccccacaagcctcgctgaaacccaggaagaccacaatgggggccagaagcagctgtccccacaaaaagtgacgctttacctgcgaccaggccaggcagcagcgttcaacgtgaccttccggcgggccaagggctaccccatcgacctgtactatctgatggacctctcctactccatgcttgatgacctcaggaatgtcaagaagctaggtggcgacctgctccgggccctcaacgagatcaccgagtccggccgcattggcttcgggtccttcgtggacaagaccgtgctgccgttcgtgaacacgcaccctgataagctgcgaaacccatgccccaacaaggagaaagagtgccagcccccgtttgccttcaggcacgtgctgaagctgaccaacaactccaaccagtttcagaccgaggtcgggaagcagctgatttccggaaacctggatgcacccgagggtgggctggacgccatgatgcaggtcgccgcctgcccggaggaaatcggctggcgcaacgtcacgcggctgctggtgtttgccactgatgacggcttccatttcgcgggcgacgggaagctgggcgccatcctgacccccaacgacggccgctgtcacctggaggacaacttgtacaagaggagcaacgaattcgactacccatcggtgggccagctggcgcacaagctggctgaaaacaacatccagcccatcttcgcggtgaccagtaggatggtgaagacctacgagaaactcaccgagatcatccccaagtcagccgtgggggagctgtctgaggactccagcaatgtggtccatctcattaagaatgcttacaataaactctcctccagggtcttcctggatcacaacgccctccccgacaccctgaaagtcacctacgactccttctgcagcaatggagtgacgcacaggaaccagcccagaggtgactgtgatggcgtgcagatcaatgtcccgatcaccttccaggtgaaggtcacggccacagagtgcatccaggagcagtcgtttgtcatccgggcgctgggcttcacggacatagtgaccgtgcaggttcttccccagtgtgagtgccggtgccgggaccagagcagagaccgcagcctctgccatggcaagggcttcttggagtgcggcatctgcaggtgtgacactggctacattgggaaaaactgtgagtgccagacacagggccggagcagccaggagctggaaggaagctgccggaaggacaacaactccatcatctgctcagggctgggggactgtgtctgcgggcagtgcctgtgccacaccagcgacgtccccggcaagctgatatacgggcagtactgcgagtgtgacaccatcaactgtgagcgctacaacggccaggtctgcggcggcccggggagggggctctgcttctgcgggaagtgccgctgccacccgggctttgagggctcagcgtgccagtgcgagaggaccactgagggctgcctgaacccgcggcgtgttgagtgtagtggtcgtggccggtgccgctgcaacgtatgcgagtgccattcaggctaccagctgcctctgtgccaggagtgccccggctgcccctcaccctgtggcaagtacatctcctgcgccgagtgcctgaagttcgaaaagggcccctttgggaagaactgcagcgcggcgtgtccgggcctgcagctgtcgaacaaccccgtgaagggcaggacctgcaaggagagggactcagagggctgctgggtggcctacacgctggagcagcaggacgggatggaccgctacctcatctatgtggatgagagccgagagtgtgtggcaggccccaacatcgccgccatcgtcgggggcaccgtggcaggcatcgtgctgatcggcattctcctgctggtcatctggaaggctctgatccacctgagcgacctccgggagtacaggcgctttgagaaggagaagctcaagtcccagtggaacaatgataatccccttttcaagagcgccaccacgacggtcatgaaccccaagtttgctgagagttaggagcacttggtgaagacaaggccgtcaggacccaccatgtctgccccatcacgcggccgagacatggcttgccacagctcttgaggatgtcaccaattaaccagaaatccagttattttccgccctcaaaatgacagccatggccggccgggtgcttctgggggctcgtcggggggacagctccactctgactggcacagtctttgcatggagacttgaggagggagggcttgaggttggtgaggttaggtgcgtgtttcctgtgcaagtcaggacatcagtctgattaaaggtggtgccaatttatttacatttaaacttgtcagggtataaaatgacatcccattaattatattgttaatcaatcacgtgtatagaaaaaaaataaaacttcaatacaggctgtccatggaaaaaaaaaaaaaaaaaa

SEQ ID NO: 31 - DNA, 2755 bp, Homo sapiens
cctacccgcgcgcaggccaagttgctgaatcaatggagccctccccaacccgggcgttccccagcgaggcttccttcccatcctcctgaccaccggggcttttcgtgagctcgtctctgatctcgcgcaagagtgacacacaggtgttcaaagacgcttctggggagtgagggaagcggtttacgagtgacttggctggagcctcaggggcgggcactggcacggaacacaccctgaggccagccctggctgcccaggcggagctgcctcttctcccgcgggttggtggacccgctcagtacggagttggggaagctctttcacttcggaggattgctcaacaaccatgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaagagaaaggaagtacagaaaacatgcagaaagcacagaaaggaaaaccaaggttctcatgaatctccaactttaaatcctgaaacagtggcaataaatttatctgatgttgacttgagtaaatatatcaccactattgctggagtcatgacactaagtcaagttaaaggctttgttcgaaagaatggtgtcaatgaagccaaaataggtgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctagagtgaaaaacaacaaattcagttctgagtatatgcaattagtgtttgaaaagattcttaatagctggctgtaaatactgcttggttttttactgggtacattttatcatttattagcgctgaagagccaacatatttgtagatttttaatatctcatgattctgcctccaaggatgtttaaaatctagttgggaaaacaaacttcatcaagagtaaatgcagtggcatgctaagtacccaaataggagtgtatgcagaggatgaaagattaagattatgctctggcatctaacatatgattctgtagtatgaatgtaatcagtgtatgttagtacaaatgtctatccacaggctaaccccactctatgaatcaatagaagaagctatgaccttttgctgaaatatcagttactgaacaggcaggccactttgcctctaaattacctctgataattctagagattttaccatatttctaaactttgtttataactctgagaagatcatatttatgtaaagtatatgtatttgagtgcagaatttaaataaggctctacctcaaagacctttgcacagtttattggtgtcatattatacaatatttcaattgtgaattcacatagaaaacattaaattataatgtttgactattatatatgtgtatgcattttactggctcaaaactacctacttctttctcaggcatcaaaagcattttgagcaggagagtattactagagctttgccacctctccatttttgccttggtgctcatcttaatggcctaatgcacccccaaacatggaaatatcaccaaaaaatacttaatagtccaccaaaaggcaagactgcccttagaaattctagcctggtttggagatactaactgctctcagagaaagtagctttgtgacatgtcatgaacccatgtttgcaatcaaagatgataaaatagattcttatttttcccccacccccgaaaatgttcaataatgtcccatgtaaaacctgctacaaatggcagcttatacatagcaatggtaaaatcatcatctggatttaggaattgctcttgtcatacccccaagtttctaagatttaagattctccttactactatcctacgtttaaatatctttgaaagtttgtattaaatgtgaattttaagaaataatatttatatttctgtaaatgtaaactgtgaagatagttataaactgaagcagatacctggaaccacctaaagaacttccatttatggaggatttttttgccccttgtgtttggaattataaaatataggtaaaagtacgtaattaaataatgtttttggtaaaaaaaaaaaaaaaaaaaaaaaaaaaaaaaaaaaaaaaaaaaaaaaaaaaaaaaaaaaaaaaaaaa

SEQ ID NO: 32 - DNA, 1280 bp, Homo sapiens
cctataacttggaatgtgggtggaggggttcatagttctccctgagtgagacttgcctgcttctctggcccctggtcctgtcctgttctccagcatggtgtgtctgaagctccctggaggctcctgcatgacagcgctgacagtgacactgatggtgctgagctccccactggctttgtctggggacacccgaccacgtttcctgtggcagcctaagagggagtgtcatttcttcaatgggacggagcgggtgcggttcctggacagatacttctataaccaggaggagtccgtgcgcttcgacagcgacgtgggggagttccgggcggtgacggagctggggcggcctgacgctgagtactggaacagccagaaggacatcctggagcaggcgcgggccgcggtggacacctactgcagacacaactacggggttgtggagagcttcacagtgcagcggcgagtccaacctaaggtgactgtatatccttcaaagacccagcccctgcagcaccacaacctcctggtctgctctgtgagtggtttctatccaggcagcattgaagtcaggtggttcctgaacggccaggaagagaaggctgggatggtgtccacaggcctgatccagaatggagactggaccttccagaccctggtgatgctggaaacagttcctcgaagtggagaggtttacacctgccaagtggagcacccaagcgtgacaagccctctcacagtggaatggagagcacggtctgaatctgcacagagcaagatgctgagtggagtcgggggctttgtgctgggcctgctcttccttggggccgggctgttcatctacttcaggaatcagaaaggacactctggacttcagccaacaggattcctgagctgaaatgcagatgaccacattcaaggaagaactttctgccccggctttgcaggatgaaaagctttcctgcttggcagttattcttccacaagagagggctttctcaggacctggttgctactggttcggcaactgcagaaaatgtcctcccttgtggcttcctcagctcctgcccttggcctgaagtcccagcattgatggcagcgcctcatcttcaacttttgtgctcccctttgcctaaaccgtatggcctcccgtgcatctgtattcaccctgtatgacaaacacattacattattaaatgtttctcaaagatggagttaaaaaaaaaaaaaaaaaaaaaaaaaaaaaaaaaaaaaaaaaaaaaaaaaaaaaaaaaaaaaaaaaa

SEQ ID NO: 33 - DNA, 2398 bp, Homo sapiens
ctgatatagagcaggcgccgcgggtcgcagcacagtgcggagaccgcagccccggagcccgggccagggtccacctgtccccgcagcgccggctcgcgccctcctgccgcagccaccgagccgccgtctagcgccccgacctcgccaccatgagagccctgctggcgcgcctgcttctctgcgtcctggtcgtgagcgactccaaaggcagcaatgaacttcatcaagttccatcgaactgtgactgtctaaatggaggaacatgtgtgtccaacaagtacttctccaacattcactggtgcaactgcccaaagaaattcggagggcagcactgtgaaatagataagtcaaaaacctgctatgaggggaatggtcacttttaccgaggaaaggccagcactgacaccatgggccggccctgcctgccctggaactctgccactgtccttcagcaaacgtaccatgcccacagatctgatgctcttcagctgggcctggggaaacataattactgcaggaacccagacaaccggaggcgaccctggtgctatgtgcaggtgggcctaaagccgcttgtccaagagtgcatggtgcatgactgcgcagatggaaaaaagccctcctctcctccagaagaattaaaatttcagtgtggccaaaagactctgaggccccgctttaagattattgggggagaattcaccaccatcgagaaccagccctggtttgcggccatctacaggaggcaccgggggggctctgtcacctacgtgtgtggaggcagcctcatcagcccttgctgggtgatcagcgccacacactgcttcattgattacccaaagaaggaggactacatcgtctacctgggtcgctcaaggcttaactccaacacgcaaggggagatgaagtttgaggtggaaaacctcatcctacacaaggactacagcgctgacacgcttgctcaccacaacgacattgccttgctgaagatccgttccaaggagggcaggtgtgcgcagccatcccggactatacagaccatctgcctgccctcgatgtataacgatccccagtttggcacaagctgtgagatcactggctttggaaaagagaattctaccgactatctctatccggagcagctgaaaatgactgttgtgaagctgatttcccaccgggagtgtcagcagccccactactacggctctgaagtcaccaccaaaatgctgtgtgctgctgacccacagtggaaaacagattcctgccagggagactcagggggacccctcgtctgttccctccaaggccgcatgactttgactggaattgtgagctggggccgtggatgtgccctgaaggacaagccaggcgtctacacgagagtctcacacttcttaccctggatccgcagtcacaccaaggaagagaatggcctggccctctgagggtccccagggaggaaacgggcaccacccgctttcttgctggttgtcatttttgcagtagagtcatctccatcagctgtaagaagagactgggaagataggctctgcacagatggatttgcctgtgccacccaccagggcgaacgacaatagctttaccctcaggcataggcctgggtgctggctgcccagacccctctggccaggatggaggggtggtcctgactcaacatgttactgaccagcaacttgtctttttctggactgaagcctgcaggagttaaaaagggcagggcatctcctgtgcatgggtgaagggagagccagctcccccgacggtgggcatttgtgaggcccatggttgagaaatgaataatttcccaattaggaagtgtaacagctgaggtctcttgagggagcttagccaatgtgggagcagcggtttggggagcagagacactaacgacttcagggcagggctctgatattccatgaatgtatcaggaaatatatatgtgtgtgtatgtttgcacacttgtgtgtgggctgtgagtgtaagtgtgagtaagagctggtgtctgattgttaagtctaaatatttccttaaactgtgtggactgtgatgccacacagagtggtctttctggagaggttataggtcactcctggggcctcttgggtcccccacgtgacagtgcctgggaatgtattattctgcagcatgacctgtgaccagcactgtctcagtttcactttcacatagatgtccctttcttggccagttatcccttccttttagcctagttcatccaatcctcactgggtggggtgaggaccactcctgtacactgaatatttatatttcactatttttatttatatttttgtaattttaaataaaagtgatcaataaaatgtgatttttctgatgacaaaaaaaaaaaaaaaaaaaa

SEQ ID NO: 34 - DNA, 3050 bp, Homo sapiens
ggagtcagtgatttgaacgaagtactttcagtttcatattactctaaatccattacaaatctgcttagcttctaaatatttcatcaatgaggaaatcccagccctacaacttcggaacagtgaaatattagtccagggatccagtgagagacacagaagtgctagaagccagtgctcgtgaactaaggagaaaaagaacagacaagggaacagcctggacatggcatcagagatccacatgacaggcccaatgtgcctcattgagaacactaatgggcgactgatggcgaatccagaagctctgaagatcctttctgccattacacagcctatggtggtggtggcaattgtgggcctctaccgcacaggcaaatcctacctgatgaacaagctggctggaaagaaaaagggcttctctctgggctccacggtgcagtctcacactaaaggaatctggatgtggtgtgtgccccaccccaagaagccaggccacatcctagttctgctggacaccgagggtctgggagatgtagagaagggtgacaaccagaatgactcctggatcttcgccctggccgtcctcctgagcagcaccttcgtgtacaatagcataggaaccatcaaccagcaggctatggaccaactgtactatgtgacagagctgacacatagaatccgatcaaaatcctcacctgatgagaatgagaatgaggttgaggattcagctgactttgtgagcttcttcccagactttgtgtggacactgagagatttctccctggacttggaagcagatggacaacccctcacaccagatgagtacctgacatactccctgaagctgaagaaaggtaccagtcaaaaagatgaaacttttaacctgcccagactctgtatccggaaattcttcccaaagaaaaaatgctttgtctttgatcggcccgttcaccgcaggaagcttgcccagctcgagaaactacaagatgaagagctggaccccgaatttgtgcaacaagtagcagacttctgttcctacatctttagtaattccaaaactaaaactctttcaggaggcatccaggtcaacgggcctcgtctagagagcctggtgctgacctacgtcaatgccatcagcagtggggatctgccgtgcatggagaacgcagtcctggccttggcccagatagagaactcagctgcagtgcaaaaggctattgcccactatgaacagcagatgggccagaaggtgcagctgcccacagaaaccctccaggagctgctggacctgcacagggacagtgagagagaggccattgaagtcttcatcaggagttccttcaaagatgtggaccatctatttcaaaaggagttagcggcccagctagaaaaaaagcgggatgacttttgtaaacagaatcaggaagcatcatcagatcgttgctcagctttacttcaggtcattttcagtcctctagaagaagaagtgaaggcgggaatttattcgaaaccagggggctatcgtctctttgttcagaagctacaagacctgaagaaaaagtactatgaggaaccgaggaaggggatacaggctgaagagattctgcagacatacttgaaatccaaggagtctatgactgatgcaattctccagacagaccagactctcacagaaaaagaaaaggagattgaagtggaacgtgtgaaagctgagtctgcacaggcttcagcaaaaatgttgcaggaaatgcaaagaaagaatgagcagatgatggaacagaaggagaggagttatcaggaacacttgaaacaactgactgagaagatggagaacgacagggtccagttgctgaaagagcaagagaggaccctcgctcttaaacttcaggaacaggagcaactactaaaagagggatttcaaaaagaaagcagaataatgaaaaatgagatacaggatctccagacgaaaatgagacgacgaaaggcatgtaccataagctaaagaccagagccttcctgtcacccctaaccaaggcataattgaaacaattttagaatttggaacaagcgtcactacatttgataataattagatcttgcatcataacaccaaaagtttataaaggcatgtggtacaatgatcaaaatcatgttttttcttaaaaaaaaaaaaagactgtaaattgtgcaacaaagatgcatttacctctgtatcaactcaggaaatctcataagctggtaccactcaggagaagtttattcttccagatgaccagcagtagacaaatggatactgagcagagtcttaggtaaaagtcttgggaaatatttgggcattggtctggccaagtctacaatgtcccaatatcaaggacaaccaccctagcttcttagtgaagacaatgtacagttatccgttagatcaagactacacggtctatgagcaataatgtgatttctggacattgcccatgtataatcctcactgatgatttcaagctaaagcaaaccaccttatacagagatctagaatctctttatgttctccagaggaaggtggaagaaaccatgggcaggagtaggaattgagtgataaacaattgggctaatgaagaaaacttctcttattgttcagttcatccagattataacttcaatgggacactttagaccattagacaattgacactggattaaacaaattcacataatgccaaatacacaatgtatttatagcaacgtataatttgcaaagatggactttaaaagatgctgtgtaactaaactgaaataattcaattacttattatttagaatgttaaagcttatgatagtcttttctaactcttaacactcatacttgaaaactttctgagtttccccagaagagaatatgggattttttttgacatttttgactcatttaataatgctcttgtgtttacctagtatatgtagactttgtcttatgtgtgaaaagtcctaggaaagtggttgatgtttcttatagcaattaaaaattatttttgaactgaaaatacaatgtatttcac

SEQ ID NO: 35 - DNA, 4146 bp, Homo sapiens
cggagcctcagcgcccgccgcgtccctccctccgcccctttcgcccacacctacccgcccccgccggcccggctctcagtagcgtcgcccgaggctgcagcagcgcatcccggggcatggcgcggcgggggcgcggagggctcggttcggagggggccgggagcccgggcgccctggagtgaggaggaccgggagctggctctggaggctgcggaggcgacgccggagagaacgaagcctcggctgggagcggatctttcgaagatggtttggctgccttggagatttggagatctgatgccacgatgaggactcacacacggggggctcccagtgtgtttttcatatatttgctttgctttgtgtcagcctacatcaccgacgagaacccagaagttatgattcccttcaccaatgccaactacgacagccatcccatgctgtacttctccagggcagaagtggcggagctgcagctcagggctgccagctcgcacgagcacattgcagcccgcctcacggaggctgtgcacacgatgctgtccagccccttggaatacctccctccctgggatcccaaggactacagtgcccgctggaatgaaatttttggaaacaacttgggtgccttggcaatgttctgtgtgctgtatcctgagaacattgaagcccgagacatggccaaagactacatggagaggatggcagcgcagcctagttggttggtgaaagatgctccttgggatgaggtcccgcttgctcactccctggttggttttgccactgcttatgacttcttgtacaactacctgagcaagacacaacaggagaagtttcttgaagtgattgccaatgcctcagggtatatgtatgaaacttcatacaggagaggatggggatttcaatacctgcacaatcatcagcccaccaactgtatggctttgctcacgggaagcctagtcctgatgaatcaaggatatcttcaagaagcctacttatggaccaaacaagttctgaccatcatggagaaatctctggtcttgctcagggaggtgacggatggctccctctatgaaggagttgcgtatggcagctacaccactagatcactcttccaatacatgtttctcgtccagaggcacttcaacatcaaccactttggccatccgtggcttaaacaacactttgcatttatgtatagaaccatcctgccagggtttcaaaggactgtggctattgcggactcaaattacaactggttttatggtccagaaagccaattagtgttccttgataaatttgtcatgcgtaatggcagtggtaactggctagctgaccaaatcagaaggaaccgtgtggtggaaggtccaggaacaccatccaaagggcagcgctggtgcactctgcacacagaatttctctggtatgatggcagcttgaaatcggttcctcctccagactttggcacccctacactgcattattttgaagactggggtgtcgtgacttatggaagtgcactacctgcagaaatcaatagatctttcctttccttcaagtctggaaaactggggggacgtgcaatatatgacattgtccacagaaacaaatacaaagattggatcaaaggatggagaaattttaatgcagggcatgaacatcctgatcaaaactcatttacttttgctcccaatggtgtgcctttcattactgaggctctgtacgggccaaagtacaccttcttcaacaatgttttgatgttttccccagctgtgtcaaagagctgcttttctccctgggtgggtcaggtcacagaagactgctcatcaaaatggtctaaatacaagcatgacctggcagctagttgtcaggggagggtggttgcagcagaggagaaaaatggggtggttttcatccgaggagaaggtgtgggagcttataacccccagctcaacctgaagaatgttcagaggaatctcatcctcctacatccacagctgcttctccttgtagaccaaatacacctgggagaggagagtcccttggagacagcagcgagcttcttccataatgtggatgttccttttgaggagactgtggtagatggtgtccatggggctttcatcaggcagagagatggtctctataaaatgtactggatggacgatactggctacagcgagaaagcaacctttgcctcagtgacatatcctcggggctatccctacaacgggacaaactatgtgaatgtcaccatgcacctccgaagtcccatcaccagggcagcttacctcttcatagggccatctatagatgttcagagcttcactgtccacggagactctcagcaactggatgtgttcatagccaccagcaaacatgcctacgccacatacctgtggacaggtgaggccacaggacagtctgcctttgcacaggtcattgctgatcgtcacaaaattctgtttgaccggaattcagccatcaagagcagcattgtccctgaggtgaaggactatgctgctattgtggaacagaacttgcagcattttaaaccagtgtttcagctgctggagaagcagatactgtcccgagtccggaacacagctagctttaggaagactgctgaacgcctgctgagattttcagataagagacagactgaggaggccattgacaggatttttgccatatcacagcaacagcagcagcaaagcaagtcaaagaaaaaccgaagggcaggcaaacgctataaatttgtggatgctgtccctgatatttttgcacagattgaagtcaatgagaaaaagattagacagaaagctcagattttggcacagaaagaactacccatagatgaagatgaagaaatgaaagaccttttagattttgcagatgtaacatacgagaaacataaaaatgggggcttgattaaaggccggtttggacaggcacggatggtgacaactacacacagcagggccccatcactgtctgcttcctataccaggttgttcctgattctgaacattgctattttctttgtcatgttggcaatgcaactgacttatttccagagggcccagagcctacatggccaaagatgtctttatgcagttcttctcatagatagctgtattttattatggttgtactcttcttgttcccaatcacagtgttagcactgaagctataaattacctggtcattttgtgatcacaagagtctatgcaaaaaaaaaaatttctttaccccagattatcagatttttttccctcagattcattttaacaaattaagggaagatattttgacacaagaaagcaggaacgtggagaaattggagcaggaaaagaaattatcaaagcaatagaaatagcttggtggtcctatggtgtttttggaagtatttggcattgctaattgagcagtccatatagtactacttttagaagaaacaaaaagtctattttttaaagtaatgttttttcttatgagaaaaaggtttagatagaattgggttttattaatattaatttaatgctattagcaatttccatatactatattgtggaaaagactgaagaatacaattctgagaaatataaaaaaattttaatggtatactcatgttgaaagataaatgttgctaagtcctggtatgatggtgtgagcttccttggggaagtacttcttgagttatgtaactaacaggatgttttactacagatctggatggctattcagataacatggcaaaaaatgatagcagaagatcattaaaaacttaaaatatattttattagaaaacatttatctatgaatgaatatttccttgatgctggtctctgcacacatatgcttggttacttgcatgcattcattggttgttcaataagtgagatgattacagataatactgtattttccttatatggaaaaccgttatagacccaataacaactaaacctttcaaaagaaaatattttctattatgaatgttgattttcataccaaagaagatggagagtctaaaatttggatatgattcttatgtttttttaatagaaaaccttcttcaagtttattttcctaaataaacatcataattgtgaattttctctagtaaaaaaaaaaaaaaaaaaa

SEQ ID NO: 36 - DNA, 1236 bp, Homo sapiens
caggcgctgacgaggagcccggctgagggaggatgcgccgctgacgcctgcgggagccgcgcgcctggggcgggaggatgctccagaggggcctctggccgtggcgcacgcggctgctgccgacccctggcacctggcgcccagcgcgcccgtggccgctgccgcctccgccccaggttttgcgtgtgaagctgtgtggaaatgtgaaatactaccagtcacaccattatagtaccgtggtgccacctgatgaaataacagttatttatagacatggccttcccttggtaacacttaccttgccatctagaaaagaacgttgtcaattcgtagtcaaaccaatgttgtcaacagttggttcattccttcaggacctacaaaatgaagataagggtatcaaaactgcagccatcttcacagcagatggcaacatgatttcagcttctaccttgatggatattttgctaatgaatgattttaaacttgtcattaataaaatagcatatgatgtgcagtgtccaaagagagaaaaaccaagtaatgagcacactgctgagatggaacacatgaaatccttggttcacagactatttacaatcttgcatttagaagagtctcagaaaaagagagagcaccatttactggagaaaattgaccacctgaaggaacagctgcagccccttgaacaggtgaaagctggaatagaagctcattcggaagccaaaaccagtggactcctgtgggctggattggcactgctgtccattcagggtggggcactggcctggctcacgtggtgggtgtactcctgggatatcatggagccagttacatacttcatcacatttgcaaattctatggtcttttttgcatactttatagtcactcgacaggattatacttactcagctgttaagagtaggcaatttcttcagttcttccacaagaaatcaaagcaacagcactttgatgtgcagcaatacaacaagttaaaagaagaccttgctaaggctaaagaatccctgaaacaggcgcgtcattctctctgtttgcaaatgcaagtagaagaactcaatgaaaagaattaatcttacagttttaaatgtcgtcagattttccattatgtattgattttgcaacttaggatgtttttgagtcccatggttcattttgattgtttaatctttgttattaaattcttgtaaaacagaaaaaaaaaaaaaaaaaaaaaaaaaa

SEQ ID NO: 37 - DNA, 2805 bp, Homo sapiens
ctccttgcacgggccggcccagcttccccgcccctggcgtccgctccctcccgctcgcagcttacttaacctggcccgggcggcggaggcgctctcacttccctggagccgcccgcttgcccgtcggtcgctagctcgctcggtgcgcgtcgtcccgctccatggcgctcttcgtgcggctgctggctctcgccctggctctggccctgggccccgccgcgaccctggcgggtcccgccaagtcgccctaccagctggtgctgcagcacagcaggctccggggccgccagcacggccccaacgtgtgtgctgtgcagaaggttattggcactaataggaagtacttcaccaactgcaagcagtggtaccaaaggaaaatctgtggcaaatcaacagtcatcagctacgagtgctgtcctggatatgaaaaggtccctggggagaagggctgtccagcagccctaccactctcaaacctttacgagaccctgggagtcgttggatccaccaccactcagctgtacacggaccgcacggagaagctgaggcctgagatggaggggcccggcagcttcaccatcttcgcccctagcaacgaggcctgggcctccttgccagctgaagtgctggactccctggtcagcaatgtcaacattgagctgctcaatgccctccgctaccatatggtgggcaggcgagtcctgactgatgagctgaaacacggcatgaccctcacctctatgtaccagaattccaacatccagatccaccactatcctaatgggattgtaactgtgaactgtgcccggctgctgaaagccgaccaccatgcaaccaacggggtggtgcacctcatcgataaggtcatctccaccatcaccaacaacatccagcagatcattgagatcgaggacacctttgagacccttcgggctgctgtggctgcatcagggctcaacacgatgcttgaaggtaacggccagtacacgcttttggccccgaccaatgaggccttcgagaagatccctagtgagactttgaaccgtatcctgggcgacccagaagccctgagagacctgctgaacaaccacatcttgaagtcagctatgtgtgctgaagccatcgttgcggggctgtctgtagagaccctggagggcacgacactggaggtgggctgcagcggggacatgctcactatcaacgggaaggcgatcatctccaataaagacatcctagccaccaacggggtgatccactacattgatgagctactcatcccagactcagccaagacactatttgaattggctgcagagtctgatgtgtccacagccattgaccttttcagacaagccggcctcggcaatcatctctctggaagtgagcggttgaccctcctggctcccctgaattctgtattcaaagatggaacccctccaattgatgcccatacaaggaatttgcttcggaaccacataattaaagaccagctggcctctaagtatctgtaccatggacagaccctggaaactctgggcggcaaaaaactgagagtttttgtttatcgtaatagcctctgcattgagaacagctgcatcgcggcccacgacaagagggggaggtacgggaccctgttcacgatggaccgggtgctgacccccccaatggggactgtcatggatgtcctgaagggagacaatcgctttagcatgctggtagctgccatccagtctgcaggactgacggagaccctcaaccgggaaggagtctacacagtctttgctcccacaaatgaagccttccgagccctgccaccaagagaacggagcagactcttgggagatgccaaggaacttgccaacatcctgaaataccacattggtgatgaaatcctggttagcggaggcatcggggccctggtgcggctaaagtctctccaaggtgacaagctggaagtcagcttgaaaaacaatgtggtgagtgtcaacaaggagcctgttgccgagcctgacatcatggccacaaatggcgtggtccatgtcatcaccaatgttctgcagcctccagccaacagacctcaggaaagaggggatgaacttgcagactctgcgcttgagatcttcaaacaagcatcagcgttttccagggcttcccagaggtctgtgcgactagcccctgtctatcaaaagttattagagaggatgaagcattagcttgaagcactacaggaggaatgcaccacggcagctctccgccaatttctctcagatttccacagagactgtttgaatgttttcaaaaccaagtatcacactttaatgtacatgggccgcaccataatgagatgtgagccttgtgcatgtgggggaggagggagagagatgtactttttaaatcatgttccccctaaacatggctgttaacccactgcatgcagaaacttggatgtcactgcctgacattcacttccagagaggacctatcccaaatgtggaattgactgcctatgccaagtccctggaaaaggagcttcagtattgtggggctcataaaacatgaatcaagcaatccagcctcatgggaagtcctggcacagtttttgtaaagcccttgcacagctggagaaatggcatcattataagctatgagttgaaatgttctgtcaaatgtgtctcacatctacacgtggcttggaggcttttatggggccctgtccaggtagaaaagaaatggtatgtagagcttagatttccctattgtgacagagccatggtgtgtttgtaataataaaaccaaagaaacata

SEQ ID NO: 38 - DNA, 1227 bp, Homo sapiens
ctttgcagataaatatggcacactagccccacgttttctgagacattcctcaattgcttagacatattctgagcctacagcagaggaacctccagtctcagcaccatgaatcaaactgccattctgatttgctgccttatctttctgactctaagtggcattcaaggagtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctccttaaaaccagaggggagcaaaatcgatgcagtgcttccaaggatggaccacacagaggctgcctctcccatcacttccctacatggagtatatgtcaagccataattgttcttagtttgcagttacactaaaaggtgaccaatgatggtcaccaaatcagctgctactactcctgtaggaaggttaatgttcatcatcctaagctattcagtaataactctaccctggcactataatgtaagctctactgaggtgctatgttcttagtggatgttctgaccctgcttcaaatatttccctcacctttcccatcttccaagggtactaaggaatctttctgctttggggtttatcagaattctcagaatctcaaataactaaaaggtatgcaatcaaatctgctttttaaagaatgctctttacttcatggacttccactgccatcctcccaaggggcccaaattctttcagtggctacctacatacaattccaaacacatacaggaaggtagaaatatctgaaaatgtatgtgtaagtattcttatttaatgaaagactgtacaaagtagaagtcttagatgtatatatttcctatattgttttcagtgtacatggaataacatgtaattaagtactatgtatcaatgagtaacaggaaaattttaaaaatacagatagatatatgctctgcatgttacataagataaatgtgctgaatggttttcaaaataaaaatgaggtactctcctggaaatattaagaaagactatctaaatgttgaaagatcaaaaggttaataaagtaattataactaagaaaaaaaaaaaa

SEQ ID NO: 39 - DNA, 586 bp, Homo sapiens
agttaaaagggtgggagcgtccgggggcccatctctctcgggtggagtcttctgacagctggtgcgcctgcccgggaacatcctcctggactcaatcatggcttgtggtctggtcgccagcaacctgaatctcaaacctggagagtgccttcgagtgcgaggcgaggtggctcctgacgctaagagcttcgtgctgaacctgggcaaagacagcaacaacctgtgcctgcacttcaaccctcgcttcaacgcccacggcgacgccaacaccatcgtgtgcaacagcaaggacggcggggcctgggggaccgagcagcgggaggctgtctttcccttccagcctggaagtgttgcagaggtgtgcatcaccttcgaccaggccaacctgaccgtcaagctgccagatggatacgaattcaagttccccaaccgcctcaacctggaggccatcaactacatggcagctgacggtgacttcaagatcaaatgtgtggcctttgactgaaatcagccagcccatggcccccaataaaggcagctgcctctgctccctctgaaaaaaaaaaaaaaaaaaaaaaaaaaaaaa

SEQ ID NO: 40 - DNA, 2078 bp, Homo sapiens
ttttccgcccggctcccggcaagggtcccccgactggcgcccgcgcgtcctccctcggctgctgcaggccgggccgcggcgtcgagcgggggcggcggggcggggcccgcagccattggcgagcggcggggcgggggcgggggcgcggagggtcggccccgggacgcgcgcagccggcccgcagttgccgctgtcgtccgcagagccagttcctagcgcagagccgcgcccgccatgagggagatcgtgcacatccaggcgggccagtgcgggaaccagatcggcaccaagttttgggaagtgatcagcgatgagcacggcatcgacccggccggaggctacgtgggagactcggcgctgcagctggagagaatcaacgtctactacaatgagtcatcgtctcagaaatatgtgcccagggccgccctggtggacttagagccaggcaccatggacagcgtgcggtctgggccttttgggcagcttttccggcctgacaacttcatctttggccagacgggtgcagggaacaactgggcgaaagggcactacacggagggcgcggagctggtggacgcagtgctggacgtggtgcggaaggagtgcgagcactgcgactgcctgcagggcttccagctcacgcactcgctgggcggcggcacgggctcaggcatgggcacgctgctcatcagcaagatccgtgaggagttcccggaccgcatcatgaacaccttcagcgtcatgccctcgcccaaggtgtcggacacggtggtggagccctacaatgccacactgtcggtgcaccagctggtggagaatacagacgagacctactgcatcgacaacgaggcgctctatgacatctgcttccgcactctgaagctgacaacgcccacctacggggacctcaaccacctggtgtccgccaccatgagtggggtcaccacctcgctgcgcttcccgggccagctcaatgctgacctgcgcaagctggcggtgaacatggtgcccttcccgcgcctgcacttcttcatgcctggcttcgcgccgctcaccagccgcggcagccagcagtaccgggccctgaccgtgcccgagctcacccagcagatgttcgacgccaggaacatgatggccgcctgcgatccgcgccatggccgctacctgaccgtggccaccgtgttccgcgggcccatgtccatgaaggaggtggacgagcagatgctggccatccagagtaagaacagcagctacttcgtggagtggattcccaacaacgtgaaggtggccgtgtgcgacatcccgccccgcggcctgaagatggcctccaccttcatcggcaacagcacggccatccaggagctgttcaagcgcatctccgagcagttctcagccatgttccggcgcaaggccttcctgcactggttcacgggtgagggcatggatgaaatggagttcaccgaggcggagagcaacatgaacgacctggtatccgagtaccagcagtaccaggatgccaccgccaatgacggggaggaagcttttgaggatgaggaagaggagatcgatggatagtcggaatagagccgccccaactcagatcctacaacacgcaagttccttcttgaaccctggtgcctcctaccctatggccctgaatggtgcactggtttaattgtgttggtgtcggcccctcacaaatgcagccaagtcatgtaattagtcatctggaacaaagactaaaaacagcagagaattgcgggttctacccagtcagaagatcacaccatggagactttctactagaggacttgaaagagaactgaggggccacaaaataaacttcaccttccattaagtgttcaagcatgtctgcaaattaggagggagttagaaacagtctttttcatcctttgtgatgaagcctgaaattgtgccgtgttgccttatatgaatatgcagtatgggactttgaaataatgattcataataaaatactaaacgtgtgtcttcatctctctagccatgtgcataaatgacgtaagttactttgtatggttaaaaaaaaaaaaaaaaaa

SEQ ID NO: 41 - DNA, 1623 bp, Homo sapiens
ctggtggcatagggcagacacgcctgcagacattctctgggaaagggcagcagcagccaggtgtggcagtgacagggaggtgtgaatgaggcaggatgaactggacaggtttgtacaccttgctcagtggcgtgaaccggcattctactgccattggccgagtatggctctcggtcatcttcatcttcagaatcatggtgctggtggtggctgcagagagtgtgtggggtgatgagaaatcttccttcatctgcaacacactccagcctggctgcaacagcgtttgctatgaccaattcttccccatctcccatgtgcggctgtggtccctgcagctcatcctagtttccaccccagctctcctcgtggccatgcacgtggctcaccagcaacacatagagaagaaaatgctacggcttgagggccatggggaccccctacacctggaggaggtgaagaggcacaaggtccacatctcagggacactgtggtggacctatgtcatcagcgtggtgttccggctgttgtttgaggccgtcttcatgtatgtcttttatctgctctaccctggctatgccatggtgcggctggtcaagtgcgacgtctacccctgccccaacacagtggactgcttcgtgtcccgccccaccgagaaaaccgtcttcaccgtcttcatgctagctgcctctggcatctgcatcatcctcaatgtggccgaggtggtgtacctcatcatccgggcctgtgcccgccgagcccagcgccgctccaatccaccttcccgcaagggctcgggcttcggccaccgcctctcacctgaatacaagcagaatgagatcaacaagctgctgagtgagcaggatggctccctgaaagacatactgcgccgcagccctggcaccggggctgggctggctgaaaagagcgaccgctgctcggcctgctgatgccacataccaggcaacctcccatcccacccccgaccctgccctgggcgagcccctccttctcccctgccggtgcacaggcctctgcctgctggggattactcgatcaaaaccttccttccctggctacttcccttcctcccggggccttccttttgaggagctggaggggtggggagctagaggccacctatgccagtgctcaaggttactgggagtgtgggctgcccttgttgcctgcacccttccctcttccctctccctctctctgggaccactgggtacaagagatgggatgctccgacagcgtctccaattatgaaactaatcttaaccctgtgctgtcagataccctgtttctggagtcacatcagtgaggagggatgtgggtaagaggagcagagggcaggggtgctgtggacatgtgggtggagaagggagggtggccagcactagtaaaggaggaatagtgcttgctggccacaaggaaaaggaggaggtgtctggggtgagggagttagggagagagaagcaggcagataagttggagcaggggttggtcaaggccacctctgcctctagtccccaaggcctctctctgcctgaaatgttacacattaaacaggattttacagtaaatgaagaggtggcttgtgaaaaaaaaaaaaaaaaaaaa

SEQ ID NO: 42 - DNA, 3310 bp, Homo sapiens
gcgcgggccgggccgggcggcggcggcggcaccatgagcggccggaagcgcagcttcaccttcggggcctacggcggggtggacaagtccttcacttctcgccggagtgtgtggaggagcgatgggcagaaccagcacttccctcaggcactagacctgtcacgagtgaacttagttccctcctatactccttcactctaccctaagaacacagatctatttgagatgattgagaagatgcagggaagcaggatggatgaacaacgctgctccttcccgccgcccctcaaaacagaggaggactacattccatacccgagcgtgcacgaggtcttggggcgagaaggacccttccccctcatcctgctgccccagtttgggggctactggattgagggcaccaaccacgaaatcaccagcatccccgagacagagccactgcagtcgcccacaaccaaggtgaagctcgagtgcaaccccacagcccgcatctaccggaagcactttctcggcaaggagcatttcaattactactcactggacgctgccctcggccaccttgtcttctcactcaagtacgatgtcatcggggaccaagagcacctgcggctgctgctcaggaccaagtgccggacataccatgatgtcatccccatctcctgcctcaccgagttccctaatgttgtccagatggcaaagttggtgtgtgaagacgtcaatgtggatcggttctatcctgtgctctaccccaaggcttcccggctcatcgtcacctttgacgagcatgtcatcagcaataacttcaagtttggcgtcatttatcagaagcttgggcagacctccgaggaagaactcttcagcaccaatgaggaaagtcccgctttcgtggagttccttgaatttcttggccagaaggtcaaactgcaggactttaaggggttccgaggaggcctggacgtgacccacgggcagacggggaccgaatctgtgtactgcaacttccgcaacaaggagatcatgtttcacgtgtccaccaagctgccatacacggaaggggacgcccagcagttgcagcggaagcggcacatcgggaacgacatcgtggctgtggtcttccaggatgagaacactcctttcgtgcccgacatgatcgcgtccaacttcctgcatgcctacgtcgtggtgcaggctgagggcgggggccctgatggccccctctacaaggtctctgtcactgcaagagatgatgtgcccttctttggaccccccctcccggaccccgctgtgttcaggaaggggcctgagttccaggaatttttgctgacaaagctgatcaatgctgaatatgcctgctacaaggcagagaagtttgccaaactggaggagcggacgcgggccgccctcctggagacgctctatgaggaactacacatccacagccagtccatgatgggcttgggcggcgacgaggacaagatggagaatggcagtgggggcggcggcttctttgagtctttcaagcgggtcatccggagccgcagccagtccatggatgccatggggctgagcaacaagaagcccaacaccgtgtccaccagccacagcgggagcttcgcgcccaacaaccccgacctggccaaggcggctggaatatcactgattgtccctgggaagagccccacgaggaagaagtcgggcccgttcggctcccgccgcagcagcgccattggcatcgagaacatacaggaggtgcaggagaagagggagagccctccggctggtcagaagaccccagacagcgggcacgtctcacaggagcccaagtcggagaactcatccactcagagctccccagagatgcccacgaccaagaacagagcggagaccgcagcgcagagagcagaggcgctcaaggacttctcccgctcctcgtccagtgccagcagcttcgccagcgtggtggaggagacggagggtgtggacggagaggacacaggcctggagagcgtgtcatcctcaggaacaccccacaagcgggactccttcatctatagcacgtggctggaggacagtgtcagcaccactagtgggggcagctccccaggcccctctcgatcaccccacccagacgccggcaagttgggggaccctgcgtgtcccgagatcaagatccagctggaagcatctgagcagcacatgccccagctgggctgttagccgggccaccccctctgaaggtgaaactgagcagatgaggccacagaagcacaaggggaaggtgccgtgtcaagcccaggcagacgagacctctgccctgaagaccaacaccagcccgtgggctgccccctgcctccccaccctccccatggcccacccatctgggctgtctctgcagggcagagccgtccagacctgggatcagggaagctgctggcatcgtccccacccccagcctgggggtctgggctggggcagggattgctcagtggaagcaggactgggggtctggcttgccccctccctgggcctccatcacccctgagcatccctctggactcagagggaacaaggtgggagagagagtttgagacagctccgtgtggagagcttagcccctggaggcagcacaaggaggatgtgatatgtgggggagtgagcactgggttgggagccgggtcctggtttccaatttgggttctgctgtgtgactctgggcaagtcactctccctctctgggcatgtctgctacaaatggacaagattatttcagaggtcactgaagactgtgattacatgcacctgccttagaaggtaggattttcttcccagggacctcctatcaccctaccctgcttcttgaggtccctggagccccaggtgggctgaggggcagggagccggctgtgcccagtatgcctcctggaccctccagttctgccacaggtctgccgatgccctgtccactgcctacacatgacagacaagtaaccccctcatgggggatggggacctacctggctcctcagccagcacccagcttaacccctgccatcccatgctgggccctccaggccaagagtctcagctggccgagagtccaggccttgcctcccccgaccgccatggagggggcagcccggcacagctgctgggagcccttgtgtgtctggtcacactttttaggcgtcacgccaaaggccagcctcctggccccaatacccattttggaagcccctgtggccgtgtggatgtcggtaacagttgtataaaataaattctatttatcgctattgtaaaaaaaaaaaaaaaa

SEQ ID NO: 43 - DNA, 5665 bp, Homo sapiens
gcgcagcccgcagaggcgctgcggcccgtgcagccccggaggcccctcgcggagaaggcggcggcggaggagaggccgagttaccgcccgccgcccgcgcccccccaaccccgccgccgccgccgccgccgccactgccccccctccccgcggcgccgcatcttgaatggaaacatggcggtgccggctcggacctgcggcgcctctcggcccggcccagcgcggactgcgcgcccctggcccggctgcggcccccaccctggccccggcacccggcgcccgacgtccgggcccccgcgcccgctgtggctgctgctgccgcttctaccgctgctcgccgcccccggcgcctctgcctacagcttcccccagcagcacacgatgcagcactgggcccggcgtctggagcaggaggtcgacggcgtgatgcggatttttggaggcgtccagcagctccgtgagatttacaaggacaaccggaacctgttcgaggtacaggagaatgagcctcagaagttggtggagaaggtggcaggggacattgagagccttctggacaggaaggtgcaggccctgaagagactggctgatgctgcagagaacttccagaaagcacaccgctggcaggacaacatcaaggaggaagacatcgtgtactatgacgccaaggctgacgctgagctggacgaccctgagagtgaggatgtggaaagggggtctaaggccagcaccctaaggctggacttcatcgaggacccaaacttcaagaacaaggtcaactattcatacgcggctgtacagatccctacggacatctacaaaggctccactgtcatcctcaatgagctcaactggacagaggccctggagaatgtgttcatggaaaaccgcagacaagaccccacactgctgtggcaggtcttcggcagcgccacaggagtcactcgctactacccggccaccccgtggcgagcccccaagaagatcgacctgtacgatgtccgaaggagaccctggtatatccagggggcctcgtcacccaaagacatggtcatcatcgtggatgtgagtggcagtgtgagcggcctgaccctgaagctgatgaagacatctgtctgcgagatgctggacacgctgtctgatgatgactatgtgaatgtggcctcgttcaacgagaaggcacagcctgtgtcatgcttcacacacctggtgcaggccaatgtgcgcaacaagaaggtgttcaaggaagctgtgcagggcatggtggccaagggcaccacaggctacaaggccggctttgagtatgcctttgaccagctgcagaactccaacatcactcgggccaactgcaacaagatgatcatgatgttcacggatggtggtgaggaccgcgtgcaggacgtctttgagaagtacaattggccaaaccggacggtgcgcgtgtttactttctccgtggggcagcataactatgacgtcacaccgctgcagtggatggcctgtgccaacaaaggctactattttgagatcccttccatcggagccatccgcatcaacacacaggaatatctagatgtgttgggcaggcccatggtgctggcaggcaaggaggccaagcaggtgcagtggaccaacgtgtatgaggatgcactgggactggggttggtggtaacagggaccctccctgttttcaacctgacacaggatggccctggggaaaagaagaaccagctgatcctgggcgtgatgggcattgacgtggctctgaatgacatcaagaggctgacccccaactacacgcttggagccaacggctatgtgtttgccattgacctgaacggctacgtgttgctgcaccccaatctcaagccccagaccaccaacttccgggagcctgtgactctggacttcctggatgcggagctagaggatgagaacaaggaagagatccgtcggagcatgattgatggcaacaagggccacaagcagatcagaacgttggtcaagtccctggatgagaggtacatagatgaggtgacacggaactacacctgggtgcctataaggagcactaactacagcctggggctggtgctcccaccctacagcaccttctacctccaagccaatctcagtgaccagatcctgcaggtcaagtattttgagttcctgctccccagcagctttgagtctgaaggacacgttttcattgctcccagagagtactgcaaggacctgaatgcctcagacaacaacaccgagttcctgaaaaactttattgagctcatggagaaagtgactccagactccaagcagtgcaacaacttccttctgcacaacctgatcttggacacgggcatcacgcagcagctggtagagcgtgtgtggagggaccaggatctcaacacgtacagcctactggccgtgttcgctgccacagacggtggcatcacccgagtcttccccaacaaggcagctgaggactggacagagaaccctgagcccttcaatgccagcttctaccgccgcagcctggataaccacggttatgtcttcaagcccccacaccaggatgccctgttaaggccgctggagctggagaatgacactgtgggcatcctcgtcagcacagctgtggagctcagcctaggcaggcgcacactgaggccagcagtggtgggcgtcaagctggacctagaggcttgggctgagaagttcaaggtgctagccagcaaccgtacccaccaagaccagcctcagaagtgcggccccaacagccactgtgagatggactgcgaggttaacaatgaggacttactctgtgtcctcattgatgatggaggattcctggtgctgtcaaaccagaaccatcagtgggaccaggtgggcaggttcttcagtgaggtggatgccaacctgatgctggcactctacaataactccttctacacccgcaaggagtcctatgactatcaggcagcctgtgcccctcagccccctggcaacctgggtgctgcaccccggggtgtctttgtgcccaccgttgcagatttccttaacctggcctggtggacctctgctgccgcctggtccctgttccagcagcttctctacggcctcatctaccacagctggttccaagcagaccccgcggaggccgaggggagccccgagacgcgcgagagcagctgcgtcatgaaacagacccagtactacttcggctcggtaaacgcctcctacaacgccatcatcgactgcggaaactgctccaggctgttccacgcgcagagactgaccaacaccaatcttctctttgtggtggccgagaagccgctgtgcagccagtgcgaggctggccggctgctgcagaaggagacgcactgcccagcggacggcccggagcagtgtgagctagtgcagagaccgcgataccggagaggcccgcacatctgcttcgactacaacgcgacagaagatacctcagactgtggccgcggggcctccttcccgccgtcgctgggcgtcctggtctccctgcaactgctgctcctcctgggcctgccgccccggccgcagcctcaagtcctcgtccacgcctctcgccgcctctgagcaccctgccccaccccacctccactcccacctcacccggcctcttcgcctttcccaccctcctgccccacactccccgccttagagcctcgtccctccctcactgaaggacctgagctggccaggccctgagagtctggtctgcgccttgggatggggagtcccaaagcgggacgccgcaggtgtttggcacccaaatcacatctcacctccgaactgttcaagtgtccccagacccttcttgcctgctgggctccccccagtgggatgggacagggaggccacacgcactggtgccaaaaccaggcctctgctgccgcccttcctggaggctgcctatgttgggggggaccctgcctcagctgacccggcctctctgccccacccaagcccaaacttggtttctgtgagaatagtggaggaaggtgagatggccagtttgaagcctgtgcctcccagcttaaatcctagcaggagagaggctctggggcagcccccatgggctcctgcccctttcaggcctacagccacatccccaagcccaccaggtgtcaggatagtcacagtgataccagttcagacactaccccatatacacctggaacattgaggatggaaactggactcacattcgacataccccactgggcacacgcacaaacacacacactatggggtggggtgggtgtaggggcttacaaagccttacacagggcgaggggttggtgggagggttggcacctgcacactccatctcctgctcaccacctgcctctaatctgagctgcagcctggctggtcctcccatttctaaagctgaatgtcaaacagtgccaaatgctggggcagggggtgaagaaccctctgtcccacccctagccaccagtgtcctccaagtgccccctcacctctccaggtgctcattgtaaccatttctcactagtgtcaggcccccagtgggaccacatgccactgcctgcacctttcggcagaggaacccccaccagacatcaccctttgccttagcaggggtgactttgtctctcctggctgggccatccttccgccaatctggcccttacacactcaggcctgtgcccactccctatctccttcccacccctacacacacactccctgcttgcaggaggccaaactgtccctcccttgctgaacacacacacacacacacacacacaggtggggactgggcacagctcttcacaccattcattctggtcatttcccccaaaggcatcccagcctgggggccagtggggaactgagggcaaggggatatagtgatggggctcagatggactgggaggagggggagggtgatgcattaattaatggcttcgttaattaatgtcatgttgcttgtcgctttctcagtgtgtgtgtgtggtccatgcccactgctggtgccagggtgggtgtccatgtgcacccggcctggatgccagctgtgtccttcgggggcgtgcgtgtaactgtagtgtagtcaggtgctcaatggagaatataaacatatacagaaaaatatatattttaagtttaaaaaacagaaaaacagacaaaacaatccccatcaggtagctgtctaacccccagctgggtctaatccttctcattacccacccgacctggctgcccctcaccttgggctgggggactggggggccatttccttttctctgccctttttttgttgttctattttgtacagacaagttggaaaaacaacagcgacaaaaaagtcaagaaactttgtaaaatatcgtgtgtgtgattccttgtaaaatattttcaaatggtttattacagaagatcagttattaaataatgttcatattttcacttcaaatggttcccatccactgtatcagcttgggggtgaggactgggtagctatgaagacagttggccaagacctcagagtcccacttagtgctctgcagggggtgaagaccatggtagcccaggtccctgtcaaccacagggcatggcacttgcttccaccagttataataggtgccaggcccttgacata

SEQ ID NO: 44 - DNA, 1768 bp, Homo sapiens
tataaattccctcccttcgctccttccccggaacagcggcctctgacaccagcacagcaaacccgccgggatcaaagtgtaccagtcggcagcatggctacgaaatgtgggaattgtggacccggctactccacccctctggaggccatgaaaggacccagggaagagatcgtctacctgccctgcatttaccgaaacacaggcactgaggccccagattatctggccactgtggatgttgaccccaagtctccccagtattgccaggtcatccaccggctgcccatgcccaacctgaaggacgagctgcatcactcaggatggaacacctgcagcagctgcttcggtgatagcaccaagtcgcgcaccaagctggtgctgcccagtctcatctcctctcgcatctatgtggtggacgtgggctctgagccccgggccccaaagctgcacaaggtcattgagcccaaggacatccatgccaagtgcgaactggcctttctccacaccagccactgcctggccagcggggaagtgatgatcagctccctgggagacgtcaagggcaatggcaaagggggttttgtgctgctggatggggagacgttcgaggtgaaggggacatgggagagacctgggggtgctgcaccgttgggctatgacttctggtaccagcctcgacacaatgtcatgatcagcactgagtgggcagctcccaatgtcttacgagatggcttcaaccccgctgatgtggaggctggactgtacgggagccacttatatgtatgggactggcagcgccatgagattgtgcagaccctgtctctaaaagatgggcttattcccttggagatccgcttcctgcacaacccagacgctgcccaaggctttgtgggctgcgcactcagctccaccatccagcgcttctacaagaacgagggaggtacatggtcagtggagaaggtgatccaggtgccccccaagaaagtgaagggctggctgctgcccgaaatgccaggcctgatcaccgacatcctgctctccctggacgaccgcttcctctacttcagcaactggctgcatggggacctgaggcagtatgacatctctgacccacagagaccccgcctcacaggacagctcttcctcggaggcagcattgttaagggaggccctgtgcaagtgctggaggacgaggaactaaagtcccagccagagcccctagtggtcaagggaaaacgggtggctggaggccctcagatgatccagctcagcctggatgggaagcgcctctacatcaccacgtcgctgtacagtgcctgggacaagcagttttaccctgatctcatcagggaaggctctgtgatgctgcaggttgatgtagacacagtaaaaggagggctgaagttgaaccccaacttcctggtggacttcgggaaggagccccttggcccagcccttgcccatgagctccgctaccctgggggcgattgtagctctgacatctggatttgaactccaccctcatcacccacactccctattttgggccctcacttccttggggacctggcttcattctgctctctcttggcacccgacccttggcagcatgtaccacacagccaagctgagactgtggcaatgtgttgagtcatatacatttactgaccactgttgcttgttgctcactgtgctgcttttccatgagctcttggaggcaccaagaaataaactcgtaaccctgtccttcagaaaaaaaaaaaaaaaa

SEQ ID NO: 45 - DNA, 9306 bp, Homo sapiens
gggttcggagcgcgaagccgccgctgggtcctcggcgcgccccgcgtctgcgcttgctgccgcgccccggtcggcgcgctgggagttccagccatgctcttctggcacacgcagcccgagcactacaaccagcacaactccggcagctacctgcgtgatgtgctcgctctgcccatcttcaagcaggaggaaccccagctgtcccccgagaacgaggcccgcctgccacccctgcaatatgtgttgtgtgctgccacgtccccagccgtgaagctgcatgaagagacgctgacctacctcaaccaaggtcagtcttatgaaatccgactactggagaatcggaagctgggagactttcaagatctgaacacaaaatatgtcaagagcatcatccgtgtggtcttccatgaccgccggctgcagtatacggagcaccagcagctggagggctggcggtggagtcggccaggggaccggatcctggacatcgatattccactgtctgttggtatcttggaccccagggccagcccgacccagctgaatgcagtcgagtttttgtgggaccctgcgaagagagcttctgcattcattcaggtacactgcatcagcacagaattcacccccaggaagcacgggggcgagaagggagtgccctttcgagtccagattgacacgtttaagcagaacgagaatggggagtacacggagcacctgcactcagccagctgccagatcaaggtgttcaagccgaagggagccgatcggaaacagaagactgaccgggagaagatggagaaaagaactgcccaagagaaggagaaataccagccgtcctatgaaaccaccatcctcacagagtgctctccatggcccgacgtggcctaccaggtgaacagcgccccgtccccaagctacaatggttctccaaacagctttggcctcggcgaaggcaacgcctctccgacccacccggtggaggccctgcccgtgggcagtgaccacctgctcccatcagcttcgatccaggatgcccagcagtggcttcaccgcaacaggttctcgcagttctgccggctctttgccagcttctcaggtgctgacttgctgaagatgtcccgagatgatttggtccagatctgtggtcccgcagatgggatccggctcttcaacgccatcaaaggccggaatgtgaggccaaagatgaccatttatgtctgtcaggagctggagcagaatcgagtgcccctgcagcagaagcgggacggcagtggagacagcaacctgtctgtgtaccacgccatcttcctggaagagctgaccaccttggagctgattgagaagatcgccaacctgtacagcatctccccccagcacatccaccgagtctaccggcagggccccacgggcatccatgtggtggtgagcaacgagatggtgcagaacttccaagatgaatcctgttttgtcctcagcacaattaaagctgagagcaatgatggctaccacatcatcctgaaatgtggactctgagcagcagtggacctcatacctgtctccagctcccagccctgtggatccccgtggatgtagacattgccccactgtaagctgtggcctcaccaggcaagctgaggccaggagggaccctgcccagtctgtgaaagctacagagcaccaaccagcagaagcctgtggacaccaagtacggtgtacagaaagccagtggctcctttctcccttcctcttggcctccagattttgaatggttccttgttcttttctattggtccaaccctgacgttctaaaagggcaaacagtggagacgtctgctctgaaatccctcatcccttagttggaagctgattgggtatcttggtgctgcctgtattggtcccttctgaccactctcctgcctccagagaaagctctgcttcaccctggaagctggtacctttacctcctcctctgggagttggctgcatggccagcactgccgacttgatgggagcagtttgccctcattctcctgtttcaggtttgcttcccttctcagtgaccctggtgagcatccgcctttcctgttcttggatgaattgatgggagtggggctattctgtgccttctacctctttcttctctacgttgtttctaaggatctgctgctgcggaacccaaagatgtgctcctgtctctgcactggcgcattggcatggtagatgccacaatgtatgtgcacggcctttctcagagacattagttctgaggccctttgtggggaggttagggggatggtaatagaaaaagactattttatttcctggcaatcacgggtaaggaggattaggaatgagtattccattcctaggtgtcatcagatgaccttgaccaccacaataccaggccctcttggatggacttatagaaagttagagaagaccttgttgaaccgctgctaaacttgccacaggagcgatgtgttttctctgagtgcccctcacttacatgtttatctttgtttgtagaggctatgtttaggatattttgcctgcatcagaatgggtgcatcatctttcttaatggcctaactatcgggaaatttgagtgtcagtaactgtggtagactcagaaattcgtctttgtcttgcctctggttcctgggatccagtgatctctactggcccagggcttcagctcttggttaatttaggttcatggggaaccctctgaccacctgaatgggatgtcatagcttctaaatggagcttctgtggaatgaagtgctagactgaaggactaccagaataaaacagggtctacaatggggagaacttgttttatagatgaggaaaccaaggctcagaggggcaaagtcacctgcatggtagcacatagtgatagggtagcgatataaatttatcatataaaccaggacatctcggaataaaaggggctctgttagtcattatgttgggtaatagccgtggcattcctacagaacagagtgaggacaggctcctgattcctcttccttctttagaggagaagcggggagtgggttaactaacagctttattgagatgtcattcacatgccattcagtttacccattgctagtgtccaattgtattcacagaaccaccatcaattcacagaattacagtcaacgttggtacattttcatcacccccagtaaaaccccgtacccttggtctgtcactcctgctttcctaactcctgcagtccaaggcagccatgaatctactttctatgtaagattaacctactctggacatttcatatatctggaatcatgtgatatctcttttgtgactggcttcttccactgaatgttttctagggccgtccaagttgaggatgtatcagtacttcattcttttgtattgctgaataatacttcattgtatagatagaccacatttgtttattgattcatcagttgatggacatttgtgtgtttttactttttggctactctgaatgatgctgctatgaacatatttctacaagattttgtgtggacatatgttttcatttcttttagcaatatacataggagtggaattgctaggtcttacagtaactccgtgttttaactttttgagaaactgccagactgttttctatagcagctgtaccattttacattcccaccagcaatgtatccaggtttcaatttgtctacatcctcatcaacacttgctattatctgtctttttgcttttagcatcctaatgagtatgaaatgctatcttgtggttttgatttgcattcccctgatggcaactgatgctgagtgtcttttcctgtgcttacgggccatgcgtatttctttggagaaaggtctatccaggtcctttgcctatttttaattgagttgtctttttttttttaagttttctgttttcctaaccactagactaccagggatgagccttctttttattattgagttgggtgagctatttgtatattctagacgccagtcttttatcaggtatatgactggtaaaaatgttctccccttctgtggattgttttcagtttcttgttggtgtcctttgagacacaaaactttttaactttgatgatttccaagatacgtattttttttctattgtcacttgtgcttttggtgccatatctagaaaaccattgcctaatccaaggtcaagaagattaatgcctgtgttttcttctaagaactatacttttagttctcacaatggtctttgatccatttcgagtatatttttatatatgatgtgatgtaggggtccagcttcattcttttgcttgtggatctccacttgtcccactgctgattattgagaaaaatatcctttctccacggaattgtcttggcatccttgctaaaggcctctgcttcttactggatcttctttcctgggacatggtgtcgttgggaagcttaccttttttttttttttacttagtctgtgtttggttccaccagttttatgctgcctttctactctgttcttgctgtctccctctttacctgagtcaacggtactgagtcctatctctctctgatgttccccagtcttccttggtgcatgttctagctccacacactagtccttggaggaaggttgagaccaatgatttcctgttatgagtcatgaggaaactgaatcacctagaagtggaataatgtgctcagggtcaccatagcccattagtggaaggaccaggactagacctttagtcttctgaggtccagccccttaggctgtctgtcatcactgtacccaagtgatgtcactaccaaggccaaatgatggtgggctaaattttaattctcaaaagtgtaggaggctaatattgtcttctaagttccaaaagaagatgtaataaaagtctgttaccttaagtgtgctattagtagagtcttccatttttctggcatgcccctggcatctgctcttcttaccttctcgtggttgtagttaaagcttatagcttatgaaagaatagaaaataataaataccaaaaaaaagtacacatggtaatttggtaccaaaatatctcagctgcctaatttagcagctcatcccttccacaggggtcagatgagctaaagctccaggttttatttttcatttgattgacatacagaaaagccatagcccttcccacagctgtccagggtctttcctgtgagtccggaggtgctggcctattgagcaggacagctcttcccagggcattcccaccaacctgtggcttctgaactgtagcttctttttacagtgaaccccagagggaaataagacagacacatgtgctcaggccaccatcttgaactggaagcccaaagctgagttccttactcttaggtcgtcacggtttttgcggggtatctgcaaggttgagataaaccctttcctgtttaccaggttgtcctttctggatgaagggacagaggctgttgaatggaggaataataggtttgctggaggaggggcatggtatgcctgtggaaaggacaggatggggtggggaggtcgaggctttgacttggggtcctaaacaaaggtcaggtgttgccctagtgacctcttgcccagacagcccagagccccttacacagagctattaacctagggaaggctttaccagcagtggactggagccagccagggtcacaagtttccaagtccagcattgcttcaggggctggcctgagtaactgaagatctgaaaatcattaacaagtcgatgaaataaacggaaaagcctcttaggctgttgtcagtggagcagagggagaaagtccctaggcgctcagagggggtgagaaagcagtggatgattgggcgggggtgggggattagatgttgacactgcctggggtgtaggaagaggaacagagaacccagagtcagggtcctagatcccagaccctcgctcagtatgagtctctttgcctctctgggtctctatctcctcctcttacaaatacaggcttggtgatctctgaagatggcaccaacctgccatgaaatgaatctgaggggttttcccatttttccctccatcaaaatcgtacaaaaagctggacgtggtggcccatgcctctaatcctagcattttgggaggccgaggtgggagaatcacttgacgccaagagttcgagaccagcctgggcatcgtagtgagactccatctctgtctttttgaaaataaaaaatctttgaaaattgcacaacaggcaggagacctttacgtgtgcccatcctggttgtacacagtgccaccagtgctcctgcagtgcaaggcggcatgcttcttgacatgggtcagattgtgtccatcgtgtctttgggaatcagccctagctcctaactgggctgactacttcctccgcaaacttatgggggctcccagatattccttgccagccaggggccagacacagtgcaggcacagtctgtgtcattggtgcacatgtgcgtgtttacatgtgtacctgggttccttcccttgcccatgaatttgccatgagcacagccagaagcagcctcagcttggcaaggtgtggagatgactgctgttcccttcgcatttggggaaaacaggctccctcggtagctcgatgatcctcttttgatcttgtgtgacctcctggagagtggatgaagctggtggccttagcttttctagacagtgtaagtggcactgggcaaggcccccagagcagggcaaggtctctagagcgggtctcccacatgactggcttcacacaggcacttccgctcgggttgcatgctctgtgtcatcttaccggtccagggttgcaggtaggaaatgtttgtaccctcttctgattgccacctccttcccatcgccccttagggacagggcttgagggccagtgaggcgctggtcaggcaccccaggcctccttgggacctgcccaggggcaccctgagagctcctgaaacccccacttagcttccagacctttctgcaaaagctcctcctggctttcctccctcccccaatctatgggtcacagctaacagatctgagggcaactgctgtgctagtggccagggctgcacctgccatccccggctctgccactttagggccttctagaggcagtgtccttaggaagtagctctgaggcatgggttttctgctcctgtgcagggcagctgatgggataaggtggggaaggacggtcagtgcttgggccccagctggccagcctggcgatggggaaaccaaaccatgtcccccagcgaagggccagagtgggaacctgtcctcatgcccttcgtcctgaggagccctgaggtgggcagcaggggccaggggaagttttcaggccttcatcaaagagaacaacatcctcagctccgcacccctcatcctgtatcagcacttaccggtgtgtgactgcccttgtcagctagcatacggtgggcccacctggcccactggctgtttatgccactgatttatgatagggaatattatctttgaacccaatgaagtgttttctcccccatcacaaaaaaaaaaattcttatttttagtagacatgtatttaccaaaaatatgtactcaattattgtattttggattttatcaatttaaaaattgtggaaatttgtttgctcttacgccaacataatattgattttgcctcttggctctgaaagcccaaaatatttaccgtctagcccgttacagaaaaagtctgctgactactgagccagacctccattacctccatccctgttggattatttaaagaaagcctcagacagtaagggcttttttaaaagaataaaatgacttggtttgcgcttggaagcaggggaagcattcagatgagcggtttctgcattaaccctgcctatcacgcatctcgtgtcctgtgtggctggcgagccccccttggaaggttctggtgcttcagctggctcctgcagagtccaccccgcctcgtggtgggaatgcagagccctttgctttccttcttgccgcctgcttcctgttcctggggacccgctgggcctttggtctgcatcccctggccaggtccctcagggttgatgcgtggagaaggactttgagcagtggtgggcagcagtggcctcctggccagctcacactcttgtcctgggaggggcagcctgatctcacctccacctagtaccttggggactgaggaccttttggcttctctggagcctgcaagcctcttcccatgtgtccagctgctcttcctgctacaaaggggactgctcacagtggcctcagcttggtggttttgaggggccgccccccggccctccataagggtatcctgggcctgagaattctgcatctgccattggaggatggacagcctcaaatggaaggagtcccacgggagatgggtccgaggtccggctgtggccatccagccccctgtggcttgtccagcctctgtgcacccctggtgtcttcactccaggggcagacagcagccactgcagttcctttcttcgtgagtaacagtagtgatagcagctggggctaacaggctaggctttgtgttctgcgcatttggtcagcttctcactcgatcctccctaaagcaatggggaggcccccactagcccagttttcaggaagtcaactgggaggttagatgggggccagggtcccacagctactgatggcccgagccaggttgagcttcctggtgtccagtccggatcccacttgcagatctcatgctctcagataggtgggacaagttcttttgtcacagtgctggctctgtcctgaggcctcattgctggctgggtgtgctctgctgggaaaagctttgcggggcttgcttggttaaccacagaagagaaggggactgtttggggtgcctctctgcagcctccccgtgctgggtggaagcacggttactgtgttctctaatgttcatgtatttaaaatgatttctttctaaagatgtaacctccacacctttctccagattgggtgactcttttctaaaggtggtgggagtatctgtcggggtggtgtggcccttggatgggtcaggtgggtgtgagaggtcctggggaggtgggcgttgagctcaaagttgtcctactgccatgtttttgtacctgaaataaagcatattttgcacttgttactgtaccatagtgcggacgagaagtctgtatgtgggatctgtgcttgggttagaatgcaaataaaactcacatttgtaagaaaaaaaaaaaaaaaaaaaaaa

SEQ ID NO: 46 - DNA, 6564 bp, Homo sapiens
atgcattaaactccaggtattttcagggaggaatcatgaagccgctcctgttaaatcatatccattttattacgtctctccttgtggattacgtagaactttggaaaattaaacattagacatcttcatagaaaatcatgtcactgatttttaaagccagctcaatttaacctgccactgattccctaaccagctcttctctgactgtatcccgagggaaaatgtattgaacgcagcagaaggttaaggcagaggagttcggctagaccgggtagcagccaatcagatggggagggtccaaggatctgatgcctccaggcgagcagcgccaagctgagctggttagtctccttttattgtggatgaacaccacgtacatgccgtaaatgctccctaggctggctgcggctacaaggcagacccgggagctgagaatagaggaatacctgcccaagcaactcattctgcacagcagccgacaagacagcgattttcctgtgctggaatgtgcccatccaaagccaaccaatttgttggaaaatcagtcaaaatctaattttgatgttctgcaagttccctgggggccatgaagcacctgacacttggattggacttaatcttaaacctctggagttcaagaccttttaaaaagggctaaataaacaatctctacatgtaaaaggccactgactcctacttcctctgtatagagcaactgttgaactcagctgcctgtaggaaaactgaagactttaataacaaactctccaaggtgaaaatgaacacagatagcggtgggtgtgctcgcaaacgtgccgccatgtctgttacgctaacatctgtgaagagagtgcaaagttctccaaacctattggctgcagggcgtgattctcagtcaccagactcagcttggagatcttacaatgatggcaatcaggagacactgaacggagatgctacatattcctctcttgcagcaaaaggttttagaagcgttcgaccaaacctacaagataaaagatcaccaactcagagccagataacagtgaatggaaactcaggaggtgccgtgagtcccatgagttactatcagaggccgttttccccctcggcatattctctcccagcctcactcaactccagcattgtcatgcagcacggcacatccctcgattccacagacacatatccccagcatgcgcagtctctggatggcaccaccagcagctctatccccctgtaccgatcctcagaggaagagaagagagtgacagtcatcaaagccccgcattacccagggatcgggcccgtggatgaatccggaatccccacagcaattagaacgacagtcgaccggcccaaggactggtacaagacgatgtttaagcaaattcacatggtgcacaagccggatgatgacacagacatgtataatactccttatacatacaatgcaggtctgtacaacccaccctacagtgctcagtcacaccctgctgcaaagacccaaacctacagacctctttccaaaagccactccgacaacagccccaatgcctttaaggatgcgtcctccccagtgcctcccccacatgttccacctccagtcccgccgcttcgaccaagagatcggtcttcaacagaaaagcatgactgggatcctccagacagaaaagtggacacaagaaaatttcggtctgagccaaggagtatttttgaatatgaacctggcaagtcatcaattcttcagcatgaaagaccagcctccttgtatcagtcctctatagacagaagcctggaaagacccatgagttctgcaagcatggccagtgacttcaggaagcggaggaagagcgagcctgcagtgggtccaccacggggcttgggagatcaaagtgcgagcaggactagcccaggccgagtggacctcccaggatcaagcaccactcttacaaagtctttcactagctcttctccttcttccccatcaagagcaaaaggtggggatgatagcaaaatatgtccatccctttgcagttactcagggctcaacggcaacccctccagtgagttagattactgtagcacttacagacagcacttggatgtccctcgggactcaccaagagccattagtttcaagaacggctggcaaatggcccggcaaaacgcagaaatctggagcagcacggaagaaaccgtctctcccaaaatcaaatcccggagctgtgacgatctcctaaacgacgactgcgatagcttcccggacccgaaagtcaagtcggaaagcatgggctccctgttatgtgaggaggactccaaggagagctgccccatggcgtgggggtccccctacgtcccggaggttcgcagtaacggcagatcaaggatcagacacaggtcagcccgcaacgccccggggttcctgaagatgtacaagaaaatgcaccgcatcaaccgcaaggacctgatgaactccgaggtgatctgctccgtgaagtccaggattctgcagtacgagagcgagcagcagcacaaggacctgctgcgcgcctggagccagtgctccacggaggaggtgccccgggacatggtgcccacacgcatctccgaattcgaaaagctgatccagaagtccaaatccatgccaaatttaggggatgacatgctgtctcccgtaaccctagaaccaccgcaaaatggcctatgtcccaagaggcggttttccattgagtatttgctggaggaagaaaatcaaagcggcccccccgctcggggccggcgaggctgccagtctaacgccctggtgcccattcacattgaagtcaccagcgatgagcagccccgagcacacgtggagttttccgacagcgaccaggacggggttgtgtccgaccacagtgactacattcacctagaggggtcatccttctgcagtgaaagtgactttgatcacttttccttcacatcctccgaaagcttttacggatccagccaccaccaccaccatcaccaccaccaccaccaccgccacctcatcagctcctgcaaaggcaggtgcccggcctcctacactcgatttaccacaatgttaaaacacgaaagagccagacacgaaaacaccgaggagcccagaaggcaagaaatggaccctggcctttctaaacttgcttttctagtcagtcctgtgcctttccggaggaaaaaaaattcggctcctaagaaacagactgaaaaggcaaaatgtaaagcatctgtgtttgaggctctggactctgcccttaaagacatctgtgaccaaattaaagctgaaaagaaaagggggagcttgccggacaacagcatcctgcaccgcctcatcagtgagctgctgccagatgttcccgagaggaactcatccctgagagcgctgaggaggagccccctgcaccagcctctccacccactgcctcccgatggtgctattcattgtccaccctaccagaatgactgcgggagaatgccccgcagtgcctctttccaagacgtggacacagccaacagcagctgccaccaccaagaccgtggcggtgcactccaagaccgtgagtcccctagaagttactcatccactttgactgacatggggagaagtgcaccaagggaaagaagaggaactccagaaaaagagaaattgcctgcaaaagctgtttatgattttaaggctcagacatctaaggagttgtcatttaagaaaggagatactgtctacatcctcaggaaaattgatcaaaattggtatgagggagaacaccacgggagagtgggcatcttcccgatctcatacgtagagaaactcacacctcctgagaaagcacagcctgcaagaccacctccgccagcccagcccggagaaatcggagaagctatagccaaatacaacttcaacgcagacacaaatgtggagctgtcactgagaaagggagatagagttattcttcttaaaagagttgatcaaaactggtatgaaggtaaaatcccaggaaccaacagacaaggcatcttccctgtttcctatgtggaggtcgtcaagaagaacacaaaaggtgctgaggactaccctgaccctccaataccccacagctattctagtgataggattcacagcttgagctcaaataagccacagcgtcctgtgtttactcatgaaaatattcaaggtgggggggaaccgtttcaggctctgtataactatactcccaggaatgaagatgagctggagctcagagaaagtgatgtcattgatgtcatggaaaagtgtgatgacggctggtttgtggggacctcaagaagaaccaaattctttggtactttccccggaaactacgtcaagaggctgtgaattgcgctccctccttctgtagaggccgcctgccagccatgcacctgcgtcaacgcgcctgaaacaccccgcgggcctcccgttgtcatgccttacggtttccaatgcgccgtcaccatctccacctgccaccaaaccaccagcagagtagccgccgctgctgtgagcctggggacgacatggcaggctggtccccctccgtgaaagtgtggattcctacttcctgctctaagctttgacacgtcaaaatgtgggatcagaaagaaaaaaatcatgatatttaaaaatggtcaaatatttgaggcaaaaaaaaaaaaaaaaagtgtctccaggaggctgtccagcctcgtggctccatttcaacatctccccccaggcgatgttctcccccaagacgaccagaaaattgtttattggggaatgctgtggtttgcattttcatattcttcgcttggcagtgtgtattcttttcacaagtttgcctagtgtcttggtttacacaatatgacaactgtaactgtactttagctattgtttgcctgcacatacatgttgtaatatgcacagtgattacaacctttaaagcaagaggaggcgagttaatttggatgagtgtgattttgctgattgaatgtgagttttcaaataggagtctttttctgcaatttgtttgcattttttagaagtgcaaacagtaagtaaataaaagccttcggtaataatcatgacaatacaagaggctgagctaggctacaggggaaaactattgtgttgtaaagttgcatcgctattttatattaaaatgtaatgatcagcatcatgaacaatgagctcttagtgttttatttatctgaaaaaatgtaagtaaaagcagtgttagtgagaggtgcaaagaattattctaacagacacctgaaagtatctgtaagcaatactagtaggtaagatttcactgtgtgtacacatacacatttgagattgtatgagaacatataatccatatgatatgttgtacattttatggaaatgtaatagaatctcacacattattttatagaaatagagtacagaagcatcacaagtattaaagttggttttagcaaggattaggattactacaattatttttactataataattatttttcttctatggaactcagaatcctggctacatttgaggacaggaatatgttgagcctgattttcctggtgtgtgtaaaatatttcctaggaataaattaggcactttttagaagcaatgttaaatcattcaggttttattttctgccctgaagcagaaatttaaaaaatgatattgggacctggaaggtttaatatggttcacagtgcctgaattacacctgctccgaaaactagctttgtatttcttatgactttgcataagaactgttcatctttggatcttgagcaccttacagataaaactttttatggcatctctttcatggacagtgatattgatcttttcacaatgtacgtagcctctagaattttgtacatatgtttgctctttttttgtacagactagttgttgagaaaacaggggcgtttcctaatttggtctttatcctgcgacaacactttcagcagactagcctctccttatctcacagatcacaagcacccctagatagtgtgattctgtcagatagcatttatgcaaaaatctatgaagttaaaagatcgtagaagccaaatgaaatgtacatatctactgactgatgacaagggaatttcattaggaagaaggtaaagaaacatcgttgagtagcctaccttgatttctgtcaagttcataaccagcttcatattttaaaggcttcaggtttgaaattaagtcaactgcatgcagctttgctgataaatgaataattctctttgatgccatttatgagaaaagacttcaatatctgttgcctgtcatatttaagaaaaattactgtttctactctctgtatctgattttaaaagaaaaaactattcatacctggcttccaggtaattgactttgaattcttacaagcaaaggtcattgtgtttttcttgaatagacatctaataaattttgcttggaagtatacttcagtttttcttcttgacatttatctttataaaaattgtgtattttattccaacttgttaaactaagagaaaatgca

SEQ ID NO: 47 - DNA, 6398 bp, Homo sapiens
gtggaatgacgggagggagtcccggcagctcctacctcccagcgatggcgtcgctgtgccgcgcccagtccccagcctgccggccggtactcaccgctacccggagttcgctcagacggtgagatttggggcgggtccgaggcagcggcgggacgctacctgcgaccgggaccatgaggagctgccagacccgtggggccggtaacgagagcagtcgcggcacctgctgagaggaaagagggagcggtccggcgcggctggggcgcggcagaggcttgcccgatcctcggccatgtcactgctctgcgtgcgcgttaaaagggccaaattccagggttcaccagataaatttaacacatatgtgaccctgaaagtacagaatgtgaagagcacaactgtagcagttcgtggtgatcagccttcctgggaacaggatttcatgtttgagattagtcgcctggacctgggtctaagtgtggaggtatggaacaaaggactgatctgggacaccatggtggggactgtgtggattgcgctgaagactattcgtcagtcggatgaggaagggcctggggaatggtccacattagaggcagagacgttaatgaaagacgatgagatctgtggaactagaaacccaactcctcataaaattttgcttgatacaagatttgagttgccttttgatatcccagaggaggaagccagatattggacctacaaatgggagcaaatcaatgccttgggagctgacaatgagtattctagtcaagaagaaagccagaggaagccattgcccactgctgccgcccagtgttcttttgaagaccctgatagtgccgtcgatgaccgagatagtgactatcgcagtgagaccagcaacagcttcccacctccttaccatacagcttcccagcccaacgcttctgtgcaccagttccctgtgccggtgcgatcgccacagcagctgctacttcaaggcagttcccgggactcttgtaatgactctatgcaaagttatgaccttgattatccagagcggcgggctatcagccccaccagcagcagtaggtatggctcctcctgtaatgtgagtcaaggaagctctcagctaagtgaactagaccagtatcacgaacaagatgacgaccatcgggagacggactcgattcattcttgccacagctctcacagcctgtccagagatggccaagcaggttttggagaacaagagaaacccttggaggtgacaggtcaagcagagaaggaggcagcatgtgaacccaaggagatgaaagaagatgccacaacccaccctcccccagatctggtgctgcaaaaagaccacttcctaggtccccaggagagttttcctgaggagaatgcatcttcaccatttacccaagccagagcacattggatccgagcagttaccaaggttcgactccagctgcaggagattccagatgatggtgacccctctctgcctcagtggctcccggaagggccagccggagggctctatggcattgacagcatgccagatttacgcagaaagaagccactgccacttgtcagtgatctgtcactggtccagtctcggaaggcaggaatcacttctgcaatggctacacgcacttctcttaaggacgaagagctgaaatcccacgtgtataagaaaaccctgcaggccttaatctaccccatttcgtgcaccactcctcataactttgaggtctggacggccactaccccaacctactgctatgagtgtgaaggcctgctctggggcattgcccggcagggcatgcgctgcagcgaatgtggagtcaagtgccatgagaagtgccaggatctgctcaatgctgactgcctgcagcgggctgcagaaaagagctgtaaacatggagctgaggaccggacccagaacattatcatggccatgaaggaccgcatgaagatccgagagcgaaataagccagagatctttgaagttatccgggacgtcttcacagtgaacaaagctgcccatgtgcagcagatgaaaacagtgaagcagagtgtactggatggcacctccaaatggtcagccaagatcaccattactgtggtgtgtgcccagggcctacaagccaaggacaaaacaggatccagtgacccttacgtgactgtgcaagtcagcaaaactaagaagcgtaccaagaccatttttggaaacttgaatcctgtttgggaggagaagttccattttgagtgccacaactcctctgaccgcattaaggtgcgtgtatgggatgaggatgatgacatcaagtcaagagtaaagcaacgcctaaagcgagagtctgatgatttccttggccaaaccatcattgaggttcggaccctaagtggcgagatggacgtctggtacaacttggagaagaggacagacaaatcagccgtctcaggggctatccgactacaaatcagtgtggagatcaagggggaggagaaagtagccccataccacgtgcagtatacatgtctccatgagaatcttttccattacctcacagacattcagggcagtggaggagtccgcatccctgaagctcgaggagacgatgcctggaaggtgtactttgatgagacagcccaagaaattgtggatgaatttgccatgcgttatggcattgagtccatatatcaggccatgacgcactttgcatgtttatcatccaagtacatgtgtcctggtgtgccagcagtgatgagcaccttactggccaacatcaacgcctactatgcccacacaactgcctctaccaatgtctctgcatctgatcgctttgcagcctccaactttgggaaagagagatttgtaaaactgctggaccagctacacaactcactgaggatcgacctctctacatacaggaataatttccctgctgggagtcctgaacggcttcaggacttaaaatccacagtggatttgctgaccagcattactttcttcagaatgaaggtacaagaactgcaaagccctccaagagccagccaggtggtaaaggattgtgtgaaggcctgtttgaactccacatatgaatatatcttcaacaactgccacgacttatacagccgccagtaccagctgaagcaggagctacctccagaggaacaagggcccagcattcggaacctggatttctggcccaagctcatcacactcatcgtgtcaatcatagaggaagataagaattcctacacacctgttctgaaccagtttcctcaggagttgaatgtgggaaaagtcagcgcagaagtgatgtggcatttgtttgcccaagacatgaaatatgcattggaggagcatgagaaagaccacctgtgtaaaagtgctgactacatgaacctgcacttcaaggtgaagtggctccacaatgaatacgtgcgggatctgcctgtcctccaggggcaggtgcctgagtacccagcgtggtttgagcagttcgtgctacaatggctggatgagaatgaggatgtatccctggaattcctgcgtggggccctggaacgagataagaaggatggattccagcagacatcagagcatgcactcttttcctgctctgtggtggatgtcttcacacaactcaatcagagctttgagatcatccggaagctggaatgcccagaccccagcatccttgcccactacatgaggaggtttgctaagaccatcgggaaggtgctgatgcagtatgcagacatcttgtcaaaggacttcccagcctattgcacaaaggagaaactgccctgcatcctgatgaacaacgtgcagcaactgagggtccagctggagaaaatgtttgaggccatgggaggcaaggagctggaccttgaagctgcagacagtctgaaggagctgcaggtgaaactgaatacggttctggatgagctcagcatggtgtttggaaacagtttccaggtacggattgatgagtgtgttcgacaaatggccgacatcctgggccaggttcggggcacagggaatgcatctccagacgccagggcctcagcggctcaggatgcagatagcgtactccggcctctcatggacttcctggatggcaacctcaccctctttgccactgtgtgtgagaagacggttctgaagcgtgtactgaaggagctctggcgcgtggtgatgaacacaatggagaggatgattgttctgcccccactcactgaccagacgggcacccagctgatcttcactgctgccaaggagctgagccatctttccaaactcaaggatcacatggtacgagaggaaacacggaatctcactccaaagcagtgtgcagtccttgacctcgccctggacaccatcaagcaatacttccatgcaggaggcaatgggctgaagaaaaccttcctggagaagagcccagatctgcagtctctacgctatgccctgtctctgtacacacagactactgacactctcatcaagacctttgtgcgctcgcagaccacccaagggtctggtgtggacgatcctgtgggagaagtctctattcaggtggacttgtttacacaccctggtactggggagcacaaggtcacagtgaaagtggtggctgccaatgacctcaagtggcagacagcgggtatgttccggcctttcgtggaggtgactatggttggcccacaccaaagtgataagaagaggaagttcacaaccaaatccaaaagcaacaactgggcccccaagtacaatgagacattccacttcctcctgggaaatgaggaggggcccgagtcctatgagttgcagatatgcgtgaaggattactgctttgcccgggaagatcgcgtgctagggctggctgtgatgcctctgagggatgtcacagccaagggcagctgtgcctgctggtgccccttgggccggaagatccatatggatgagacaggcctgaccattctccggattttatctcagaggagcaatgacgaggtggcccgagaatttgtgaaactcaaatcagagtctcgttccacggaggaggggagctgaacaccttcgactcctgtgccaatcaggcagcagcaatttcacaaatcagggccagtgggagttagctgtgtaaccggcttagggtctttgcagtcaagaggctgaccccttcagttaaagatatttaaggaaaaatttggggtggtgataatatggcttttcacagaaagggtcatgaagccctggcccaacaggactgtggtactaggggctgggatgtggggttaccacatggagagattttccattaagagagaaggacaaacatttctgagagtgtcagccattcttggtagacacctctccactcctcatcccacctctacccatctccatgccacaccttatccagttagacacatacataccaatcattagaagaacaagtttagaaggtgtggaacttgtgcctggctggctgggtagtcagctgagcctgttgctgagcccggtggtctggattggagtatggccagggcaggagtacacagaatagaatttagactgtcccttgagtagaatccactgattttctgtggctccagtgagaacaaggctttgaaactgaacaagataacttctagaaatgaactgtactaatccctttccccagattgtatcatgagtagaatcaggttcacgtggtgcttcaaagccctgagaagaatatttctttggaccccaggcactaggggccacctgcctgggagtctccctgcctcactcctctaggcaggggagtgatgcttcaggacgtgacaggctgttctaacatgtgtctacctgagggctagttgaaggatccaggagtattttcttcttgggtgggccctgaacaaagccaaaaattgtagaaaccagtctagaaaaagtcctgctcatctgtggccactgccttctagccgtcctccaccttgcagaaagaatctagcctttggtctctctctctctcatcggggtcatttgctattcccctctgatattcaaccctatagaaggagcctggactctgatccctctgtacaggctggatggaaggggccctccacacttcctgggaggtcagagacaaactgtttcagagagtcagatggacttcccaagacttgttgagagatgtgacatggttcttggatttcctctgtagcagcctcctggacttcctgaggactcgacattgtccacagatgtactggccattacatgaaacaagaaaccaagcatctttgctgttgttaattattatatgtgccattgttacaggagattataggctagtctgtaataaattatcttatagcaaaaaaaaaaaaaaaaaaaaaaaaaaaaa

SEQ ID NO: 48 - DNA, 3311 bp, Homo sapiens
cgcccgggcaggtgaaaggttttgtctatttctgttccttgttgaatctctagtacctagaaccatgtctggtcctgattaggcacttggtgaatgtttttgaatgaatactctgagctgttctttaatcggtcctcatgatcctcattgtacagatgagggaactctgggcgaacctggcctgggtcttgagctaattacgggtaggtcaggttctgtacccaagtagtacacacagtgatgggcggggtggcctggggccgtggtttgtcaacccctacgctggagggtgatgttttggtacaagaggagaggtgccccaatgcgtcctgtgtctgtaattgatggcgttgtctgtgtttccccaggatgtggttctgagacagtccctgtccctgatggcccacggagcgactcggtggaaggaagtcccttccgtcccccgtcacactccttctctgccgtcttcgatgaagacaagccgatagccagcagtgggacttacaacttggactttgacaacattgagcttgtggatacctttcagaccttggagcctcgtgcctcagacgctaagaatcaggagggcaaagtgaacacacggaggaagtccacggattccgtccccatctctaagtctacactgtcccggtcgctcagcctgcaagccagtgactttgatggtgcttcttcctcaggcaatcccgaggccgtggcccttgccccagatgcatatagcacgggttccagcagtgcttctagtacccttaagcgaactaaaaaaccgaggccgccttccttaaaaaagaaacagaccaccaagaaacccacagagacccccccagtgaaggagacgcaacaggagccagatgaagagagccttgtccccagtggggagaatctagcatctgagacgaaaacggaatctgccaagacggaaggtcctagcccagccttattggaggagacgccccttgagcccgctgtggggcccaaagctgcctgccctctggactcagagagtgcagaaggggttgtccccccggcttctggaggtggcagagtgcagaactcaccccctgtcgggaggaaaacgctgcctcttaccacggccccggaggcaggggaggtaaccccatcggatagcggggggcaagaggactctccagccaaagggctctccgtaaggctggagtttgactattctgaggacaagagtagttgggacaaccagcaggaaaacccccctcctaccaaaaagataggcaaaaagccagttgccaaaatgcccctgaggaggccaaagatgaaaaagacacccgagaaacttgacaacactcctgcctcacctcccagatcccctgctgaacccaatgacatccccattgctaaaggtacttacacctttgatattgacaagtgggatgaccccaattttaaccctttttcttccacctcaaaaatgcaggagtctcccaaactgccccaacaatcatacaactttgacccagacacctgtgatgagtccgttgacccctttaagacatcctctaagacccccagctcaccttctaaatccccagcctcctttgagatcccagccagtgctatggaagccaatggagtggacggggatgggctaaacaagcccgccaagaagaagaagacgcccctaaagactgacacatttagggtgaaaaagtcgccaaaacggtctcctctctctgatccaccttcccaggaccccaccccagctgctacaccagaaacaccaccagtgatctctgcggtggtccacgccacagatgaggaaaagctggcggtcaccaaccagaagtggacgtgcatgacagtggacctagaggctgacaaacaggactacccgcagccctcggacctgtccacctttgtaaacgagaccaaattcagttcacccactgaggagttggattacagaaactcctatgaaattgaatatatggagaaaattggctcctccttacctcaggacgacgatgccccgaagaagcaggccttgtaccttatgtttgacacttctcaggagagccctgtcaagtcatctcccgtccgcatgtcagagtccccgacgccgtgttcagggtcaagttttgaagagactgaagcccttgtgaacactgctgcgaaaaaccagcatcctgtcccacgaggactggcccctaaccaagagtcacacttgcaggtgccagagaaatcctcccagaaggagctggaggccatgggcttgggcaccccttcagaagcgattgaaattagagaggctgctcacccaacagacgtctccatctccaaaacagccttgtactcccgcatcgggaccgctgaggtggagaaacctgcaggccttctgttccagcagcccgacctggactctgccctccagatcgccagagcagagatcataaccaaggagagagaggtctcagaatggaaagataaatatgaagaaagcaggcgggaagtgatggaaatgaggaaaatagtggccgagtatgagaagaccatcgctcagatgatagaggacgaacagagagagaagtcagtctcccaccagacggtgcagcagctggttctggagaaggagcaagccctggccgacctgaactccgtggagaagtctctggccgacctcttcagaagatatgagaagatgaaggaggtcctagaaggcttccgcaagaatgaagaggtgttgaagagatgtgcgcaggagtacctgtcccgggtgaagaaggaggagcagaggtaccaggccctgaaggtgcacgcggaggagaaactggacagggccaatgctgagattgctcaggttcgaggcaaggcccagcaggagcaagccgcccaccaggccagcctgcggaaggagcagctgcgagtggacgccctggaaaggacgctggagcagaagaataaagaaatagaagaactcaccaagatttgtgacgaactgattgccaaaatggggaaaagctaactctgaaccgaatgttttggacttaactgttgcgtgcaatatgaccgtcggcacactgctgttcctccagttccatggacaggttctgttttcactttttcgtatgcactactgtatttcctttctaaataaaattgatttgattgtatgcagtactaaggagactatcagaatttcttgctattggtttgcattttcctagtataattcatagcaagttgacctcagagttcctgtatcagggagattgtctgattctctaataaaagacacattgctgaaaaaaaaaaaaaaaaaa

SEQ ID NO: 49 - DNA, 3685 bp, Homo sapiens; identical to SEQ ID NO: 10

SEQ ID NO: 50 - DNA, 2993 bp, Homo sapiens
agcaaaccaatcgcaagcctcgttgagtggaaggggtgggatcttccccggaagtgttggttaaagcccctccaatcagcggctcggtgcggcaagtttgaatttcgtggaggctcgggttgtgagggttcctgcttcggagtcggcggtggtcgtccagaccgagtgttctttactttttgtttggttgaggtttcacgctagaaggtggctcaggatgtcttcatcacattttgccagtcgacacaggaaggatataagtactgaaatgattagaactaaaattgctcataggaaatcactgtctcagaaagaaaatagacataaggaatacgaacgaaatagacactttggtttgaaagatgtaaacattccaaccttggaaggtagaattcttgttgaattagatgagacatctcaagggcttgttccagaaaagaccaatgttaagccaagggcaatgaaaactattctaggtgatcaacgaaaacagatgctccaaaaatacaaagaagaaaagcaacttcaaaaattgaaagagcagagagagaaagctaaacgaggaatatttaaagtgggtcgttatagacctgatatgccttgttttcttttatcaaaccagaatgctgtgaaagctgagccaaaaaaggctattccatcttctgtacggattacaaggtcaaaggccaaagaccaaatggagcagactaagattgataacgagagtgatgttcgagcaatccgacctggtccaagacaaacttctgaaaagaaagtgtcagacaaagagaaaaaagttgtgcagcctgtaatgcccacgtcgttgagaatgactcgatcagctactcaagcagcaaagcaggttcccagaacagtctcatctaccacagcaagaaagccagtcacaagagctgctaatgaaaacgaaccagaaggaaaggtgccaagtaaaggaagacctgccaaaaatgtagaaacaaaacccgacaagggtatttcttgtaaagtcgatagtgaagaaaatactttgaattcacaaactaatgcaacaagtggaatgaatccagatggagtcttatcaaaaatggaaaacttacctgagataaatactgcaaaaataaaagggaagaattcctttgcacctaaggattttatgtttcagccactggatggtctgaagacctatcaagtaacacctatgactcccagaagtgccaatgcttttttgacacccagttacacctggactcctttaaaaacagaagttgatgagtctcaagcaacaaaagaaattttggcacaaaaatgtaaaacttactctaccaagacaatacagcaagattcaaataaattgccatgtcctttgggtcctctaactgtttggcatgaagaacatgttttaaataaaaatgaagctactactaaaaatttaaatggccttccaataaaagaagtcccatcacttgaaagaaatgaaggtcgaattgctcagccccaccatggtgtgccatatttcagaaatatcctccagtcagaaactgagaaattaacttcacattgcttcgagtgggacaggaaacttgaattggacattccagatgatgctaaagatcttattcgcacagcagttggtcaaacaagactccttatgaaggaaaggtttaaacagtttgaaggactggttgatgattgtgaatataaacgaggtataaaggagactacctgtacagatctggatggattttgggatatggttagttttcagatagaagatgtaatccacaaattcaacaatctgatcaaacttgaggaatctgggtggcaagtcaataataatatgaatcataatatgaacaaaaatgtctttaggaaaaaagttgtctcaggtatagcaagtaaaccaaaacaggatgatgctggaagaattgcagcgagaaatcgcctagctgccataaaaaatgcaatgagagagagaattaggcaggaagaatgtgctgaaacagcagtttctgtgataccaaaggaagttgataaaatagtgttcgatgctggatttttcagagttgaaagtcctgttaaattattctcaggactttctgtctcttctgaaggcccttctcaaagacttggaacacctaagtctgtcaacaaagctgtatctcagagtagaaatgagatgggcattccacaacaaactacatcaccagaaaatgccggtcctcagaatacgaaaagtgaacatgtgaagaagactttgtttttgagtattcctgaaagcaggagcagcatagaagatgctcagtgtcctggattaccagatttaattgaagaaaatcatgttgtaaataagacagacttgaaggtggattgtttatccagtgagagaatgagtttgcctcttcttgctggtggagtagcagatgatattaatactaacaaaaaagaaggaatttcagatgttgtggaaggaatggaactgaattcttcaattacatcacaggatgttttgatgagtagccctgaaaaaaatacagcttcacaaaatagcatcttagaagaaggggaaactaaaatttctcagtcagaactatttgataataaaagtctcactactgaatgccaccttcttgattcaccaggtctaaactgcagtaatccatttactcagctggagaggagacatcaagaacatgccagacacatttcttttggtggtaacctgattactttttcacctctacaaccaggagaattttgaatttaaaaataaatccaaacattttccttcatattatcaatgcttatatattccttagactattgaaattttggagaaaatgtatttgtgttcacttctatagcatataatgttttaatattctgtgttcatcaaagtgtattttagatatactctttctcaagggaagtggggatattttgtacattttcaacacagaataaaaaatgtactgtgccttgcctctcttgtttaa

SEQ ID NO: 51 - DNA, 3189 bp, Homo sapiens
ctatttgagtttgtggcgcgcgaggccctgcagtccgggttggcgcttgggtactggctgggtccgatgctgggtacgctgcgcgccatggagggcgaggacgtggaagacgaccagctgctgcagaagctcagggccagtcgccgccgcttccagaggcgcatgcagcggctgatagagaagtacaaccagcccttcgaggacaccccggtggtgcaaatggccacgctgacctacgagacgccacagggattgagaatttggggtggaagactaataaaggaaagaaacgaaggagagatccaggactcctccatgaagcccgcggacaggacagatggctccgtgcaagctgcagcctggggtcctgagcttccctcgcaccgcacagtcctgggagccgattcaaaaagcggtgaggtcgatgccacgtcagaccaggaagagtcagttgcttgggccttagcacctgcagtgcctcaaagccctttgaaaaatgaattaagaaggaaatacttgacccaagtggatatactgctacaaggtgcagagtattttgagtgtgcaggtaacagagctggaagggatgtacgtgtgactccgctgccttcactggcctcacctgccgtgcctgcccccggatactgcagtcgtatctccagaaagagtcctggtgacccagcgaaaccagcttcatctcccagagaatgggatcctttgcatccttcctccacagacatggccttagtacctagaaatgacagcctctccctacaagagaccagtagcagcagcttcttaagcagccagccctttgaagatgatgacatttgcaatgtgaccatcagtgacctgtacgcagggatgctgcactccatgagccggctgttgagcacaaagccatcaagcatcatctccaccaaaacgttcatcatgcaaaactggaactccaggaggaggcacagatataagagcaggatgaacaaaacatattgcaaaggagccagacgttctcagaggagctccaaggagaacttcataccctgctctgagcctgtgaaagggacaggggcattaagagattgcaagaacgtattagatgtttcttgccgtaagacaggtttaaaattggaaaaagcttttcttgaagtcaacagaccccaaatccataagttagatccaagttggaaggagcgcaaagtgacaccctcgaagtattcttccttgatttacttcgactccagtgcaacatataatcttgatgaggaaaatagatttaggacattaaaatggttaatttctcctgtaaaaatagtttccagaccaacaatacgacagggccatggagagaaccgtcagagggagattgaaatccgatttgatcagcttcatcgggaatattgcctgagtcccaggaaccagcctcgccggatgtgcctcccggactcctgggccatgaacatgtacagagggggtcctgcgagtcctggtggccttcagggcttagaaacccgcaggctgagtttaccttccagcaaagcaaaagcaaaaagtttaagtgaggcttttgaaaacctaggcaaaagatctctggaagcaggtaggtgcctgcccaagagcgattcatcttcatcacttccaaagaccaaccccacacacagcgcaactcgcccgcagcagacatctgaccttcacgttcagggaaatagttctggaatatttagaaagtcagtgtcacccagcaaaactctttcagtcccagataaagaagtgccaggccacggaaggaatcgttacgatgaaattaaagaagaatttgacaagcttcatcaaaagtattgcctcaaatctcctgggcagatgacagtgcctttatgtattggagtgtctacagataaagcaagtatggaagttcgatatcaaacagaaggcttcttaggaaaattaaatccagaccctcacttccagggtttccagaagttgccatcatcacccctggggtgcagaaaaagtctactgggctcaactgcaattgaggctccttcatctacatgtgttgctcgtgccatcacgagggatggcacgagggaccatcagttccctgcaaaaagacccaggctatcagaaccccagggctccggacgccagggcaattccctgggtgcctcagatggggtggacaacaccgtcagaccgggagaccagggcagctcttcacagcccaactcagaagagagaggagagaacacgtcttacaggatggaagagaaaagtgatttcatgctagaaaaattggaaactaaaagtgtgtagctaggttatttcggagtgttatttatcttcccacttgctctctgtttgtatttttgttttgtttttgattcttgagactgtgaggacttggttgacttctctgcccttaaagtaaatattagtgaaattggttccatcagagataacctcgagttcttggtgtagaaattatgtgaataaagttgctcaattagaatttttagggttctctttgataggcctgtttttctgatgtgtgtgttttttttgggggggggttatttgtttgtttgtttgtttgtttgtttgtttttgagacagtctctctctatcgcccaggctggagtgcggtggcacaatcttggctcactgcaacttccgcctcccgggttcaagcgattcttctgcctcagcctcccgagtagctgggattacaggcgcgcgccaccacgcctggctaatttttgtagttttagtagagacggggtttcaccatattgaccaggctggtctcgagctcctggcctcgtgatccatctgcctcggcctcccaaagtgctgggattataggcgtgagccactgctcccagccgtgtgttcttttttaaatttagatatgtccagagaatcctctctcctgtttcccatttcattcgagaatattgtttgcttgtgagacgtaagttcgagccctgcatgcaatgacccttgaaggaaaataaacagtcctggtggtcccagacgctcctgcagccacagcgcctgtgactcctcatgattcttactgaagctgttgatgacaggatatcatggtgacgtttttgtaatgaaatatttcacatattcagaatacattggtgaaactcatgctggagtaaatagttaatatatggccat

SEQ ID NO: 52 - DNA, 4504 bp, Homo sapiens; identical to SEQ ID NO: 12

SEQ ID NO: 53 - DNA, 2896 bp, Homo sapiens
acgcttgcgcgcgggatttaaactgcggcggtttacgcggcgttaagacttcgtagggttagcgaaattgaggtttcttggtattgcgcgtttctcttccttgctgactctccgaatggccatggactcgtcgcttcaggcccgcctgtttcccggtctcgctatcaagatccaacgcagtaatggtttaattcacagtgccaatgtaaggactgtgaacttggagaaatcctgtgtttcagtggaatgggcagaaggaggtgccacaaagggcaaagagattgattttgatgatgtggctgcaataaacccagaactcttacagcttcttcccttacatccgaaggacaatctgcccttgcaggaaaatgtaacaatccagaaacaaaaacggagatccgtcaactccaaaattcctgctccaaaagaaagtcttcgaagccgctccactcgcatgtccactgtctcagagcttcgcatcacggctcaggagaatgacatggaggtggagctgcctgcagctgcaaactcccgcaagcagttttcagttcctcctgcccccactaggccttcctgccctgcagtggctgaaataccattgaggatggtcagcgaggagatggaagagcaagtccattccatccgaggcagctcttctgcaaaccctgtgaactcagttcggaggaaatcatgtcttgtgaaggaagtggaaaaaatgaagaacaagcgagaagagaagaaggcccagaactctgaaatgagaatgaagagagctcaggagtatgacagtagttttccaaactgggaatttgcccgaatgattaaagaatttcgggctactttggaatgtcatccacttactatgactgatcctatcgaagagcacagaatatgtgtctgtgttaggaaacgcccactgaataagcaagaattggccaagaaagaaattgatgtgatttccattcctagcaagtgtctcctcttggtacatgaacccaagttgaaagtggacttaacaaagtatctggagaaccaagcattctgctttgactttgcatttgatgaaacagcttcgaatgaagttgtctacaggttcacagcaaggccactggtacagacaatctttgaaggtggaaaagcaacttgttttgcatatggccagacaggaagtggcaagacacatactatgggcggagacctctctgggaaagcccagaatgcatccaaagggatctatgccatggcctcccgggacgtcttcctcctgaagaatcaaccctgctaccggaagttgggcctggaagtctatgtgacattcttcgagatctacaatgggaagctgtttgacctgctcaacaagaaggccaagctgcgcgtgctggaggacggcaagcaacaggtgcaagtggtggggctgcaggagcatctggttaactctgctgatgatgtcatcaagatgatcgacatgggcagcgcctgcagaacctctgggcagacatttgccaactccaattcctcccgctcccacgcgtgcttccaaattattcttcgagctaaagggagaatgcatggcaagttctctttggtagatctggcagggaatgagcgaggcgcggacacttccagtgctgaccggcagacccgcatggagggcgcagaaatcaacaagagtctcttagccctgaaggagtgcatcagggccctgggacagaacaaggctcacaccccgttccgtgagagcaagctgacacaggtgctgagggactccttcattggggagaactctaggacttgcatgattgccacgatctcaccaggcataagctcctgtgaatatactttaaacaccctgagatatgcagacagggtcaaggagctgagcccccacagtgggcccagtggagagcagttgattcaaatggaaacagaagagatggaagcctgctctaacggggcgctgattccaggcaatttatccaaggaagaggaggaactgtcttcccagatgtccagctttaacgaagccatgactcagatcagggagctggaggagaaggctatggaagagctcaaggagatcatacagcaaggaccagactggcttgagctctctgagatgaccgagcagccagactatgacctggagacctttgtgaacaaagcggaatctgctctggcccagcaagccaagcatttctcagccctgcgagatgtcatcaaggccttgcgcctggccatgcagctggaagagcaggctagcagacaaataagcagcaagaaacggccccagtgacgactgcaaataaaaatctgtttggtttgacacccagcctcttccctggccctccccagagaactttgggtacctggtgggtctaggcagggtctgagctgggacaggttctggtaaatgccaagtatgggggcatctgggcccagggcagctggggagggggtcagagtgacatgggacactccttttctgttcctcagttgtcgccctcacgagaggaaggagctcttagttacccttttgtgttgcccttctttccatcaaggggaatgttctcagcatagagctttctccgcagcatcctgcctgcgtggactggctgctaatggagagctccctggggttgtcctggctctggggagagagacggagcctttagtacagctatctgctggctctaaaccttctacgcctttgggccgagcactgaatgtcttgtactttaaaaaaatgtttctgagacctctttctactttactgtctccctagagatcctagaggatccctactgttttctgttttatgtgtttatacattgtatgtaacaataaagagaaaaaataaatcagctgtttaagtgtgtggaaaaaaaaaaaaaaaaaa

SEQ ID NO: 54 - DNA, 2293 bp, Homo sapiens
atggtgccgcgcggcgggcgggtttggattttaaatccccgcggccaatcagtggcgcgcaggcttttgtaacgttcccagcgccgcgtttgaattcggggaggagcggagcggtgcggaggctctgctcggatcgaggtctgcagcgcagcttcgggagcatgagtgctgcagtgactgcagggaagctggcacgggcaccggccgaccctgggaaagccggggtccccggagttgcagctcccggagctccggcggcggctccaccggcgaaagagatcccggaggtcctagtggacccacgcagccggcggcgctatgtgcggggccgctttttgggcaagggcggctttgccaagtgcttcgagatctcggacgcggacaccaaggaggtgttcgcgggcaagattgtgcctaagtctctgctgctcaagccgcaccagagggagaagatgtccatggaaatatccattcaccgcagcctcgcccaccagcacgtcgtaggattccacggctttttcgaggacaacgacttcgtgttcgtggtgttggagctctgccgccggaggtctctcctggagctgcacaagaggaggaaagccctgactgagcctgaggcccgatactacctacggcaaattgtgcttggctgccagtacctgcaccgaaaccgagttattcatcgagacctcaagctgggcaaccttttcctgaatgaagatctggaggtgaaaataggggattttggactggcaaccaaagtcgaatatgacggggagaggaagaagaccctgtgtgggactcctaattacatagctcccgaggtgctgagcaagaaagggcacagtttcgaggtggatgtgtggtccattgggtgtatcatgtataccttgttagtgggcaaaccaccttttgagacttcttgcctaaaagagacctacctccggatcaagaagaatgaatacagtattcccaagcacatcaaccccgtggccgcctccctcatccagaagatgcttcagacagatcccactgcccgcccaaccattaacgagctgcttaatgacgagttctttacttctggctatatccctgcccgtctccccatcacctgcctgaccattccaccaaggttttcgattgctcccagcagcctggaccccagcaaccggaagcccctcacagtcctcaataaaggcttggagaaccccctgcctgagcgtccccgggaaaaagaagaaccagtggttcgagagacaggtgaggtggtcgactgccacctcagtgacatgctgcagcagctgcacagtgtcaatgcctccaagccctcggagcgtgggctggtcaggcaagaggaggctgaggatcctgcctgcatccccatcttctgggtcagcaagtgggtggactattcggacaagtacggccttgggtatcagctctgtgataacagcgtgggggtgctcttcaatgactcaacacgcctcatcctctacaatgatggtgacagcctgcagtacatagagcgtgacggcactgagtcctacctcaccgtgagttcccatcccaactccttgatgaagaagatcaccctccttaaatatttccgcaattacatgagcgagcacttgctgaaggcaggtgccaacatcacgccgcgcgaaggtgatgagctcgcccggctgccctacctacggacctggttccgcacccgcagcgccatcatcctgcacctcagcaacggcagcgtgcagatcaacttcttccaggatcacaccaagctcatcttgtgcccactgatggcagccgtgacctacatcgacgagaagcgggacttccgcacataccgcctgagtctcctggaggagtacggctgctgcaaggagctggccagccggctccgctacgcccgcactatggtggacaagctgctgagctcacgctcggccagcaaccgtctcaaggcctcctaatagctgccctcccctccggactggtgccctcctcactcccacctgcatctggggcccatactggttggctcccgcggtgccatgtctgcagtgtgccccccagccccggtggctgggcagagctgcatcatccttgcaggtgggggttgctgtataagttatttttgtacatgttcgggtgtgggttctacagccttgtccccctccccctcaaccccaccatatgaattgtacagaatatttctattgaattcggaactgtcctttccttggctttatgcacattaaacagatgtgaatattcaaaaaaaaaaaaaaaa

SEQ ID NO: 55 - DNA, 2486 bp, Homo sapiens; identical to SEQ ID NO: 15

SEQ ID NO: 56 - DNA, 1566 bp, Homo sapiens; identical to SEQ ID NO: 13

SEQ ID NO: 57 - DNA, 3636 bp, Homo sapiens
ggcgccctgaaacgttcggcgagccgactgcggctgcgcggggtattcgaatcggcggcggcttctagtttgcggttcaggtttggccgctgccggccagcgtcctctggccatggacaccccggaaaatgtccttcagatgcttgaagcccacatgcagagctacaagggcaatgaccctcttggtgaatgggaaagatacatacagtgggtagaagagaattttcctgagaataaagaatacttgataactttactagaacatttaatgaaggaatttttagataagaagaaataccacaatgacccaagattcatcagttattgtttaaaatttgctgagtacaacagtgacctccatcaattttttgagtttctgtacaaccatgggattggaaccctgtcatcccctctgtacattgcctgggcggggcatctggaagcccaaggagagctgcagcatgccagtgctgtccttcagagaggaattcaaaaccaggctgaacccagagagttcctgcaacaacaatacaggttatttcagacacgcctcactgaaacccatttgccagctcaagctagaacctcagaacctctgcataatgttcaggttttaaatcaaatgataacatcaaaatcaaatccaggaaataacatggcctgcatttctaagaatcagggttcagagctttctggagtgatatcttcagcttgtgataaagagtcaaatatggaacgaagagtgatcacgatttctaaatcagaatattctgtgcactcatctttggcatccaaagttgatgttgagcaggttgttatgtattgcaaggagaagcttattcgtggggaatcagaattttcctttgaagaattgagagcccagaaatacaatcaacggagaaagcatgagcaatgggtaaatgaagacagacattatatgaaaaggaaagaagcaaatgcttttgaagaacagctattaaaacagaaaatggatgaacttcataagaagttgcatcaggtggtggagacatcccatgaggatctgcccgcttcccaggaaaggtccgaggttaatccagcacgtatggggccaagtgtaggctcccagcaggaactgagagcgccatgtcttccagtaacctatcagcagacaccagtgaacatggaaaagaacccaagagaggcacctcctgttgttcctcctttggcaaatgctatttctgcagctttggtgtccccagccaccagccagagcattgctcctcctgttcctttgaaagcccagacagtaacagactccatgtttgcagtggccagcaaagatgctggatgtgtgaataagagtactcatgaattcaagccacagagtggagcagagatcaaagaagggtgtgaaacacataaggttgccaacacaagttcttttcacacaactccaaacacatcactgggaatggttcaggcaacgccatccaaagtgcagccatcacccaccgtgcacacaaaagaagcattaggtttcatcatgaatatgtttcaggctcctacacttcctgatatttctgatgacaaagatgaatggcaatctctagatcaaaatgaagatgcatttgaagcccagtttcaaaaaaatgtaaggtcatctggggcttggggagtcaataagatcatctcttctttgtcatctgcttttcatgtgtttgaagatggaaacaaagaaaattatggattaccacagcctaaaaataaacccacaggagccaggacctttggagaacgctctgtcagcagacttccttcaaaaccaaaggaggaagtgcctcatgctgaagagtttttggatgactcaactgtatggggtattcgctgcaacaaaaccctggcacccagtcctaagagcccaggagacttcacatctgctgcacaacttgcgtctacaccattccacaagcttccagtggagtcagtgcacattttagaagataaagaaaatgtggtagcaaaacagtgtacccaggcgactttggattcttgtgaggaaaacatggtggtgccttcaagggatggaaaattcagtccaattcaagagaaaagcccaaaacaggccttgtcgtctcacatgtattcagcatccttacttcgtctgagccagcctgctgcaggtggggtacttacctgtgaggcagagttgggcgttgaggcttgcagactcacagacactgacgctgccattgcagaagatccaccagatgctattgctgggctccaagcagaatggatgcagatgagttcacttgggactgttgatgctccaaacttcattgttgggaacccatgggatgataagctgattttcaaacttttatctgggctttctaaaccagtgagttcctatccaaatacttttgaatggcaatgtaaacttccagccatcaagcccaagactgaatttcaattgggttctaagctggtctatgtccatcaccttcttggagaaggagcctttgcccaggtgtacgaagctacccagggagatctgaatgatgctaaaaataaacagaaatttgttttaaaggtccaaaagcctgccaacccctgggaattctacattgggacccagttgatggaaagactaaagccatctatgcagcacatgtttatgaagttctattctgcccacttattccagaatggcagtgtattagtaggagagctctacagctatggaacattattaaatgccattaacctctataaaaatacccctgaaaaagtgatgcctcaaggtcttgtcatctcttttgctatgagaatgctttacatgattgagcaagtgcatgactgtgaaatcattcatggagacattaaaccagacaatttcatacttggaaacggatttttggaacaggatgatgaagatgatttatctgctggcttggcactgattgacctgggtcagagtatagatatgaaactttttccaaaaggaactatattcacagcaaagtgtgaaacatctggttttcagtgtgttgagatgctcagcaacaaaccatggaactaccagatcgattactttggggttgctgcaacagtatattgcatgctctttggcacttacatgaaagtgaaaaatgaaggaggagagtgtaagcctgaaggtctttttagaaggcttcctcatttggatatgtggaatgaattttttcatgttatgttgaatattccagattgtcatcatcttccatctttggatttgttaaggcaaaagctgaagaaagtatttcaacaacactatactaacaagattagggccctacgtaataggctaattgtactgctcttagaatgtaagcgttcacgaaaataaaatttggatatagacagtccttaaaaatcacactgtaaatatgaatctgctcactttaaacctgtttttttttcatttattgtttatgtaaatgtttgttaaaaataaatcccatggaatatttccatgtaacttagttgttataaatatttcaacaaaatatacaaccccataaggtccctatatagcaggctgattgggctgcttctgggatgcaagcatttgtgagaataattcagacatgagcattctctagaaatcacttt

SEQ ID NO: 58 - DNA, 3620 bp, Homo sapiens
gcagagcaccgcgccttagccgcgaagttctagttcttgctgccggtcctaacgtcccgcagtcttcgccagccagccgtcccgcatgcgcgtttgggcggcgtggagcctgctgccatgaagtcagcgagagctaagacaccccggaaacctaccgtgaaaaaagggtcccaaacgaaccttaaagacccagttggggtatactgtagggtgcgcccactgggctttcctgatcaagagtgttgcatagaagtgatcaataatacaactgttcagcttcatactcctgagggctacagactcaaccgaaatggagactataaggagactcagtattcatttaaacaagtatttggcactcacaccacccagaaggaactctttgatgttgtggctaatcccttggtcaatgacctcattcatggcaaaaatggtcttctttttacatatggtgtgacgggaagtggaaaaactcacacaatgactggttctccaggggaaggagggctgcttcctcgttgtttggacatgatctttaacagtatagggtcatttcaagctaaacgatatgttttcaaatctaatgataggaatagtatggatatacagtgtgaggttgatgccttattagaacgtcagaaaagagaagctatgcccaatccaaagacttcttctagcaaacgacaagtagatccagagtttgcagatatgataactgtacaagaattctgcaaagcagaagaggttgatgaagatagtgtctatggtgtatttgtctcttatattgaaatatataataattacatatatgatctattggaagaggtgccgtttgatcccataaaacccaaacctccacaatctaaattgcttcgtgaagataagaaccataacatgtatgttgcaggatgtacagaagttgaagtgaaatctactgaggaggcttttgaagttttctggagaggccagaaaaagagacgtattgctaatacccatttgaatcgtgagtccagccgttcccatagcgtgttcaacattaaattagttcaggctcccttggatgcagatggagacaatgtcttacaggaaaaagaacaaatcactataagtcagttgtccttggtagatcttgctggaagtgaaagaactaaccggaccagagcagaagggaacagattacgtgaagctggtaatattaatcagtcactaatgacgctaagaacatgtatggatgtcctaagagagaaccaaatgtatggaactaacaagatggttccatatcgagattcaaagttaacccatctgttcaagaactactttgatggggaaggaaaagtgcggatgatcgtgtgtgtgaaccccaaggctgaagattatgaagaaaacttgcaagtcatgagatttgcggaagtgactcaagaagttgaagtagcaagacctgtagacaaggcaatatgtggtttaacgcctgggaggagatacagaaaccagcctcgaggtccagttggaaatgaaccattggttactgacgtggttttgcagagttttccacctttgccatcatgcgaaattttggatatcaacgatgagcagacacttccaaggctgattgaagccttagagaaacgacataacttacgacaaatgatgattgatgagtttaacaaacaatctaatgcttttaaagctttgttacaagaatttgacaatgctgttttaagtaaagaaaaccacatgcaagggaaactaaatgaaaaggagaagatgatctcaggacagaaattggaaatagaacgactggaaaagaaaaacaaaactttagaatataagattgagattttagagaaaacaactactatctatgaggaagataaacgcaatttgcaacaggaacttgaaactcagaaccagaaacttcagcgacagttttctgacaaacgcagattagaagccaggttgcaaggcatggtgacagaaacgacaatgaagtgggagaaagaatgtgagcgtagagtggcagccaaacagctggagatgcagaataaactctgggttaaagatgaaaagctgaaacaactgaaggctattgttaccgaacctaaaactgagaagccagagagaccctctcgggagcgagatcgagaaaaagttactcaaagatctgtttctccatcacctgtgcctctttctagtaactatattgctcagatttccaacggccagcaactcatgagccagccacagctacataggcgctctaactcttgcagcagcatttctgtagcttcctgtatttcggaatgggagcagaaaattcctacgtacaacacacctctcaaagtcacatctattgcaaggcgtaggcagcaggagccaggacaaagcaaaacttgtatcgtgtcagacagaaggcgagggatgtactggactgaaggcagggaggtggttcctacattcagaaatgagatagaaatagaagaggatcattgcggcaggttactctttcaacctgatcagaacgcaccaccaattcgtctccgacacagacgatcacgctctgcaggagacagatgggtagatcataagcccgcctctaacatgcaaactgaaacagtcatgcagccacatgtccctcatgccatcacagtatctgttgcaaatgaaaaggcactagctaagtgtgagaagtacatgctgacccaccaggaactagcctccgatggggagattgaaactaaactaattaagggtgatatttataaaacaaggggtggtggacaatctgttcagtttactgatattgagactttaaagcaagaatcaccaaatggtagtcgaaaacgaagatcttccacagtagcacctgcccaaccagatggtgcagagtctgaatggaccgatgtagaaacaaggtgttctgtggctgtggagatgagagcaggatcccagctgggacctggatatcagcatcacgcacaacccaagcgcaaaaagccatgaactgacagtcccagtactgaaagaacattttcatttgtgtggatgatttctcgaaagccatgccagaagcagtcttccaggtcatcttgtagaactccagctttgttgaaaatcacggacctcagctacatcatacactgacccagagcaaagctttccctatggttccaaagacaactagtattcaacaaaccttgtatagtatatgttttgccatatttaatattaatagcagaggaagactccttttttcatcactgtatgaattttttataatgtttttttaaaatatatttcatgtatacttataaactaattcacacaagtgtttgtcttagatgattaaggaagactatatctagatcatgtctgattttttattgtgacttctccagccctggtctgaatttcttaaggttttataaacaaatgctgctatttattagctgcaagaatgcactttagaactatttgacaattcagactttcaaaataaagatgtaaatgactggccaataataaccattttaggaaggtgttttgaattctgtatgtatatattcactttctgacatttagatatgccaaaagaattaaaatcaaaagcactaagaaataca

SEQ ID NO: 59 - DNA, 864 bp, Homo sapiens
gagccattgattggtcgacgcccccagagggttacaattcaaacgcgggcgggcgggcccgcagtcctgcagttgcagtcgtgttctccgagttcctgtctctctgccaacgccgcccggatggcttcccaaaaccgcgacccagccgccactagcgtcgccgccgcccgtaaaggagctgagccgagcgggggcgccgcccggggtccggtgggcaaaaggctacagcaggagctgatgaccctcatgatgtctggcgataaagggatttctgccttccctgaatcagacaaccttttcaaatgggtagggaccatccatggagcagctggaacagtatatgaagacctgaggtataagctctcgctagagttccccagtggctacccttacaatgcgcccacagtgaagttcctcacgccctgctatcaccccaacgtggacacccagggtaacatatgcctggacatcctgaaggaaaagtggtctgccctgtatgatgtcaggaccattctgctctccatccagagccttctaggagaacccaacattgatagtcccttgaacacacatgctgccgagctctggaaaaaccccacagcttttaagaagtacctgcaagaaacctactcaaagcaggtcaccagccaggagccctgacccaggctgcccagcctgtccttgtgtcgtctttttaatttttccttagatggtctgtcctttttgtgatttctgtataggactctttatcttgagctgtggtatttttgttttgtttttgtcttttaaattaagcctcggttgagcccttgtatattaaataaatgcatttttgtccttttttagacaaaaaaaaaaaaaaaa

SEQ ID NO: 60 - DNA, 3471 bp, Homo sapiens
tttttccccttaagacaaagcaagcaccctaaaccagttaccctgtgcactcctgttaagattgttgctaaggaaggacaggagttggctgctgaagcctcaagatttcctttaggctcttaggtaagaaatgtctaaggttcaaggaaaaaggttaagttggaagaatcccaggcaaaataagtgcgaatccacgacagttggtaacccggacccacattagaactcagaggtcaagcagaagcgaacgactggaattccagtcaggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcggacctaggctgcccctgccgtcatgtcgcaagggatcctttctccgccagcgggcttgctgtccgatgacgatgtcgtagtttctcccatgtttgagtccacagctgcagatttggggtctgtggtacgcaagaacctgctatcagactgctctgtcgtctctacctccctagaggacaagcagcaggttccatctgaggacagtatggagaaggtgaaagtatacttgagggttaggcccttgttaccttcagagttggaacgacaggaagatcagggttgtgtccgtattgagaatgtggagacccttgttctacaagcacccaaggactcttttgccctgaagagcaatgaacggggaattggccaagccacacacaggttcaccttttcccagatctttgggccagaagtgggacaggcatccttcttcaacctaactgtgaaggagatggtaaaggatgtactcaaagggcagaactggctcatctatacatatggagtcactaactcagggaaaacccacacgattcaaggtaccatcaaggatggagggattctcccccggtccctggcgctgatcttcaatagcctccaaggccaacttcatccaacacctgatctgaagcccttgctctccaatgaggtaatctggctagacagcaagcagatccgacaggaggaaatgaagaagctgtccctgctaaatggaggcctccaagaggaggagctgtccacttccttgaagaggagtgtctacatcgaaagtcggataggtaccagcaccagcttcgacagtggcattgctgggctctcttctatcagtcagtgtaccagcagtagccagctggatgaaacaagtcatcgatgggcacagccagacactgccccactacctgtcccggcaaacattcgcttctccatctggatctcattctttgagatctacaacgaactgctttatgacctattagaaccgcctagccaacagcgcaagaggcagactttgcggctatgcgaggatcaaaatggcaatccctatgtgaaagatctcaactggattcatgtgcaagatgctgaggaggcctggaagctcctaaaagtgggtcgtaagaaccagagctttgccagcacccacctcaaccagaactccagccgcagtcacagcatcttctcaatcaggatcctacaccttcagggggaaggagatatagtccccaagatcagcgagctgtcactctgtgatctggctggctcagagcgctgcaaagatcagaagagtggtgaacggttgaaggaagcaggaaacattaacacctctctacacaccctgggccgctgtattgctgcccttcgtcaaaaccagcagaaccggtcaaagcagaacctggttcccttccgtgacagcaagttgactcgagtgttccaaggtttcttcacaggccgaggccgttcctgcatgattgtcaatgtgaatccctgtgcatctacctatgatgaaactcttcatgtggccaagttctcagccattgctagccagcttgtgcatgccccacctatgcaactgggattcccatccctgcactcgttcatcaaggaacatagtcttcaggtatcccccagcttagagaaaggggctaaggcagacacaggccttgatgatgatattgaaaatgaagctgacatctccatgtatggcaaagaggagctcctacaagttgtggaagccatgaagacactgcttttgaaggaacgacaggaaaagctacagctggagatgcatctccgagatgaaatttgcaatgagatggtagaacagatgcaacagcgggaacagtggtgcagtgaacatttggacacccaaaaggaactattggaggaaatgtatgaagaaaaactaaatatcctcaaggagtcactgacaagtttttaccaagaagagattcaggagcgggatgaaaagattgaagagctagaagctctcttgcaggaagccagacaacagtcagtggcccatcagcaatcagggtctgaattggccctacggcggtcacaaaggttggcagcttctgcctccacccagcagcttcaggaggttaaagctaaattacagcagtgcaaagcagagctaaactctaccactgaagagttgcataagtatcagaaaatgttagaaccaccaccctcagccaagcccttcaccattgatgtggacaagaagttagaagagggccagaagaatataaggctgttgcggacagagcttcagaaacttggtgagtctctccaatcagcagagagagcttgttgccacagcactggggcaggaaaacttcgtcaagccttgaccacttgtgatgacatcttaatcaaacaggaccagactctggctgaactgcagaacaacatggtgctagtgaaactggaccttcggaagaaggcagcatgtattgctgagcagtatcatactgtgttgaaactccaaggccaggtttctgccaaaaagcgccttggtaccaaccaggaaaatcagcaaccaaaccaacaaccaccagggaagaaaccattccttcgaaatttacttccccgaacaccaacctgccaaagctcaacagactgcagcccttatgcccggatcctacgctcacggcgttcccctttactcaaatctgggccttttggcaaaaagtactaaggctgtggggaaagagaagagcagtcatggccctgaggtgggtcagctactctcctgaagaaataggtctcttttatgctttaccatatatcaggaattatatccaggatgcaatactcagacactagcttttttctcacttttgtattataaccacctatgtaatctcatgttgttgtttttttttatttacttatatgatttctatgcacacaaaaacagttatattaaagatattattgttcacattttttattgaattccaaatgtagcaaaatcattaaaacaaattataaaaggga

SEQ ID NO: 61 - DNA, 2618 bp, Homo sapiens
attggacggctgagtctggctacgcgggcctccgcgggagcgcgaccgggccaatcaagagcttggcgtattttacaaactgagaaagtagctccagcagcacccgagagggtcaggagaaaagcggaggaagctgggtaggccctgaggggcctcggtaagccatcatgaccacccggcaagccacgaaggatcccctcctccggggtgtatctcctacccctagcaagattccggtacgctctcagaaacgcacgcctttccccactgttacatcgtgcgccgtggaccaggagaaccaagatccaaggagatgggtgcagaaaccaccgctcaatattcaacgccccctcgttgattcagcaggccccaggccgaaagccaggcaccaggcagagacatcacaaagattggtggggatcagtcagcctcggaaccccttggaagagctcaggcctagccctaggggtcaaaatgtggggcctgggccccctgcccagacagaggctccagggaccatagagtttgtggctgaccctgcagccctggccaccatcctgtcaggtgagggtgtgaagagctgtcacctggggcgccagcctagtctggctaaaagagtactggttcgaggaagtcagggaggcaccacccagagggtccagggtgttcgggcctctgcatatttggcccccagaacccccacccaccgactggaccctgccagggcttcctgcttctctaggctggagggaccaggacctcgaggccggacattgtgcccccagaggctacaggctctgatttcaccttcaggaccttcctttcacccttccactcgccccagtttccaggagctaagaagggagacagctggcagcagccggacttcagtgagccaggcctcaggattgctcctggagaccccagtccagcctgctttctctcttcctaaaggagaacgcgaggttgtcactcactcagatgaaggaggtgtggcctctcttggtctggcccagcgagtaccattaagagaaaaccgagaaatgtcacataccagggacagccatgactcccacctgatgccctcccctgcccctgtggcccagcccttgcctggccatgtggtgccatgtccatcaccctttggacgggctcagcgtgtaccctccccaggccctccaactctgacctcatattcagtgttgcggcgtctcaccgttcaacctaaaacccggttcacacccatgccatcaacccccagagttcagcaggcccagtggctgcgtggtgtctcccctcagtcctgctctgaagatcctgccctgccctgggagcaggttgccgtccggttgtttgaccaggagagttgtataaggtcactggagggttctgggaaaccaccggtggccactccttctggaccccactctaacagaacccccagcctccaggaggtgaagattcaacgcatcggtatcctgcaacagctgttgagacaggaagtagaggggctggtagggggccagtgtgtccctcttaatggaggctcttctctggatatggttgaacttcagcccctgctgactgagatttctagaactctgaatgccacagagcataactctgggacttcccaccttcctggactgttaaaacactcagggctgccaaagccctgtcttccagaggagtgcggggaaccacagccctgccctccggcagagcctgggcccccagaggccttctgtaggagtgagcctgagataccagagccctccctccaggaacagcttgaagtaccagagccctaccctccagcagaacccaggcccctagagtcctgctgtaggagtgagcctgagataccggagtcctctcgccaggaacagcttgaggtacctgagccctgccctccagcagaacccaggcccctagagtcctactgtaggattgagcctgagataccggagtcctctcgccaggaacagcttgaggtacctgagccctgccctccagcagaacccgggccccttcagcccagcacccaggggcagtctggacccccagggccctgccctagggtagagctgggggcatcagagccctgcaccctggaacatagaagtctagagtccagtctaccaccctgctgcagtcagtgggctccagcaaccaccagcctgatcttctcttcccaacacccgctttgtgccagcccccctatctgctcactccagtctttgagacccccagcaggccaggcaggcctcagcaatctggcccctcgaaccctagccctgagggagcgcctcaaatcgtgtttaaccgccatccactgcttccacgaggctcgtctggacgatgagtgtgccttttacaccagccgagcccctccctcaggccccacccgggtctgcaccaaccctgtggctacattactcgaatggcaggatgccctgtgtttcattccagttggttctgctgccccccagggctctccatgatgagacaaccactcctgccctgccgtacttcttccttttagcccttatttattgtcggtctgcccatgggactgggagccgcccacttttgtcctcaataaagtttctaaagta

SEQ ID NO: 62 - DNA, 1314 bp, Homo sapiens
cggggcgggagatttgaaaagtccttggccagggcgcggcgtggcagattcagttgtttgcgggcggccgggagagtagcagtgccttggaccccagctctcctccccctttctctctaaggatggcccagaaggagaactcctacccctggccctacggccgacagacggctccatctggcctgagcaccctgccccagcgagtcctccggaaagagcctgtcaccccatctgcacttgtcctcatgagccgctccaatgtccagcccacagctgcccctggccagaaggtgatggagaatagcagtgggacacccgacatcttaacgcggcacttcacaattgatgactttgagattgggcgtcctctgggcaaaggcaagtttggaaacgtgtacttggctcgggagaagaaaagccatttcatcgtggcgctcaaggtcctcttcaagtcccagatagagaaggagggcgtggagcatcagctgcgcagagagatcgaaatccaggcccacctgcaccatcccaacatcctgcgtctctacaactatttttatgaccggaggaggatctacttgattctagagtatgccccccgcggggagctctacaaggagctgcagaagagctgcacatttgacgagcagcgaacagccacgatcatggaggagttggcagatgctctaatgtactgccatgggaagaaggtgattcacagagacataaagccagaaaatctgctcttagggctcaagggagagctgaagattgctgacttcggctggtctgtgcatgcgccctccctgaggaggaagacaatgtgtggcaccctggactacctgcccccagagatgattgaggggcgcatgcacaatgagaaggtggatctgtggtgcattggagtgctttgctatgagctgctggtggggaacccaccctttgagagtgcatcacacaacgagacctatcgccgcatcgtcaaggtggacctaaagttccccgcttccgtgcccatgggagcccaggacctcatctccaaactgctcaggcataacccctcggaacggctgcccctggcccaggtctcagcccacccttgggtccgggccaactctcggagggtgctgcctccctctgcccttcaatctgtcgcctgatggtccctgtcattcactcgggtgcgtgtgtttgtatgtctgtgtatgtataggggaaagaagggatccctaactgttcccttatctgttttctacctcctcctttgtttaataaaggctgaagctttttgtactcatgaaaaaaaaaaaaaaaaaa

SEQ ID NO: 63 - DNA, 3452 bp, Homo sapiens
aaaatcgcgcgcggccccgcggccagcctgggtaggggcaaggcgcagccaatgggaagggtcggaggcatggcacagccaatgggaagggccggggcaccaaagccaatgggaagggccgggagcgcgcggcgcgggagatttaaaggctgctggagtgaggggtcgcccgtgcaccctgtcccagccgtcctgtcctggctgctcgctctgcttcgctgcgcctccactatgctctccctccgtgtcccgctcgcgcccatcacggacccgcagcagctgcagctctcgccgctgaaggggctcagcttggtcgacaaggagaacacgccgccggccctgagcgggacccgcgtcctggccagcaagaccgcgaggaggatcttccaggagcccacggagccgaaaactaaagcagctgcccccggcgtggaggatgagccgctgctgagagaaaacccccgccgctttgtcatcttccccatcgagtaccatgatatctggcagatgtataagaaggcagaggcttccttttggaccgccgaggaggtggacctctccaaggacattcagcactgggaatccctgaaacccgaggagagatattttatatcccatgttctggctttctttgcagcaagcgatggcatagtaaatgaaaacttggtggagcgatttagccaagaagttcagattacagaagcccgctgtttctatggcttccaaattgccatggaaaacatacattctgaaatgtatagtcttcttattgacacttacataaaagatcccaaagaaagggaatttctcttcaatgccattgaaacgatgccttgtgtcaagaagaaggcagactgggccttgcgctggattggggacaaagaggctacctatggtgaacgtgttgtagcctttgctgcagtggaaggcattttcttttccggttcttttgcgtcgatattctggctcaagaaacgaggactgatgcctggcctcacattttctaatgaacttattagcagagatgagggtttacactgtgattttgcttgcctgatgttcaaacacctggtacacaaaccatcggaggagagagtaagagaaataattatcaatgctgttcggatagaacaggagttcctcactgaggccttgcctgtgaagctcattgggatgaattgcactctaatgaagcaatacattgagtttgtggcagacagacttatgctggaactgggttttagcaaggttttcagagtagagaacccatttgactttatggagaatatttcactggaaggaaagactaacttctttgagaagagagtaggcgagtatcagaggatgggagtgatgtcaagtccaacagagaattcttttaccttggatgctgacttctaaatgaactgaagatgtgcccttacttggctgattttttttttccatctcataagaaaaatcagctgaagtgttaccaactagccacaccatgaattgtccgtaatgttcattaacagcatctttaaaactgtgtagctacctcacaaccagtcctgtctgtttatagtgctggtagtatcaccttttgccagaaggcctggctggctgtgacttaccatagcagtgacaatggcagtcttggctttaaagtgaggggtgaccctttagtgagcttagcacagcgggattaaacagtcctttaaccagcacagccagttaaaagatgcagcctcactgcttcaacgcagattttaatgtttacttaaatataaacctggcactttacaaacaaataaacattgtttgtactcacaaggcgataatagcttgatttatttggtttctacaccaaatacattctcctgaccactaatgggagccaattcacaattcactaagtgactaaagtaagttaaacttgtgtagactaagcatgtaatttttaagttttattttaatgaattaaaatatttgttaaccaactttaaagtcagtcctgtgtatacctagatattagtcagttggtgccagatagaagacaggttgtgtttttatcctgtggcttgtgtagtgtcctgggattctctgccccctctgagtagagtgttgtgggataaaggaatctctcagggcaaggagcttcttaagttaaatcactagaaatttaggggtgatctgggccttcatatgtgtgagaagccgtttcattttatttctcactgtattttcctcaacgtctggttgatgagaaaaaattcttgaagagttttcatatgtgggagctaaggtagtattgtaaaatttcaagtcatccttaaacaaaatgatccacctaagatcttgcccctgttaagtggtgaaatcaactagaggtggttcctacaagttgttcattctagttttgtttggtgtaagtaggttgtgtgagttaattcatttatatttactatgtctgttaaatcagaaattttttattatctatgttcttctagattttacctgtagttcatacttcagtcacccagtgtcttattctggcattgtctaaatctgagcattgtctagggggatcttaaactttagtaggaaaccatgagctgttaatacagtttccattcaaatattaatttcagaatgaaacataatttttttttttttttttgagatggagtctcgctctgttgcccaggctggagtgcagtggcgcgattttggctcactgtaacctccatctcctgggttcaagcaattctcctgtctcagcctccctagtagctgggactgcaggtatgtgctaccacacctggctaatttttgtatttttagtagagatggagtttcaccatattggtcaggctggtcttgaactcctgacctcaggtgatccacccacctcggcctcccaaagtgctgggattgcaggcgtgataaacaaatattcttaatagggctactttgaattaatctgcctttatgtttgggagaagaaagctgagacattgcatgaaagatgatgagagataaatgttgatcttttggccccatttgttaattgtattcagtatttgaacgtcgtcctgtttattgttagttttcttcatcatttattgtatagacaatttttaaatctctgtaatatgatacattttcctatcttttaagttattgttacctaaagttaatccagattatatggtccttatatgtgtacaacattaaaatgaaaggctttgtcttgcattgtgaggtacaggcggaagttggaatcaggttttaggattctgtctctcattagctgaataatgtgaggattaacttctgccagctcagaccatttcctaatcagttgaaagggaaacaagtatttcagtctcaaaattgaataatgcacaagtcttaagtgattaaaataaaactgttcttatgtcagtttcaaaaaaaaaaaaaaaaaa

SEQ ID NO: 64 - DNA, 2785 bp, Homo sapiens
actcaggcccctcccccggcgcccgcgcgccaggactgggcggcgccgacgcgcttggcgggagatagaaaagtgcttcaacccgcgccggcggcgactgcagttcctgcgagcgaggagcgcgggacctgctgacacgctgacgccttcgagcgcggcccggggcccggagcggccggagcagcccgggtcctgaccccggcccggctcccgctccgggctctgccggcgggcgggcgagcgcggcgcggtccgggccggggggatgtctcggcggacgcgctgcgaggatctggatgagctgcactaccaggacacagattcagatgtgccggagcagagggatagcaagtgcaaggtcaaatggacccatgaggaggacgagcagctgagggccctggtgaggcagtttggacagcaggactggaagttcctggccagccacttccctaaccgcactgaccagcaatgccagtacaggtggctgagagttttgaatccagaccttgtcaaggggccatggaccaaagaggaagaccaaaaagtcatcgagctggttaagaagtatggcacaaagcagtggacactgattgccaagcacctgaagggccggctggggaagcagtgccgtgaacgctggcacaaccacctcaaccctgaggtgaagaagtcttgctggaccgaggaggaggaccgcatcatctgcgaggcccacaaggtgctgggcaaccgctgggccgagatcgccaagatgttgccagggaggacagacaatgctgtgaagaatcactggaactctaccatcaaaaggaaggtggacacaggaggcttcttgagcgagtccaaagactgcaagcccccagtgtacttgctgctggagctcgaggacaaggacggcctccagagtgcccagcccacggaaggccagggaagtcttctgaccaactggccctccgtccctcctaccataaaggaggaggaaaacagtgaggaggaacttgcagcagccaccacatcgaaggaacaggagcccatcggtacagatctggacgcagtgcgaacaccagagcccttggaggaattcccgaagcgtgaggaccaggaaggctccccaccagaaacgagcctgccttacaagtgggtggtggaggcagctaacctcctcatccctgctgtgggttctagcctctctgaagccctggacttgatcgagtcggaccctgatgcttggtgtgacctgagtaaatttgacctccctgaggaaccatctgcagaggacagtatcaacaacagcctagtgcagctgcaagcgtcacatcagcagcaagtcctgccaccccgccagccttccgccctggtgcccagtgtgaccgagtaccgcctggatggccacaccatctcagacctgagccggagcagccggggcgagctgatccccatctcccccagcactgaagtcgggggctctggcattggcacaccgccctctgtgctcaagcggcagaggaagaggcgtgtggctctgtcccctgtcactgagaatagcaccagtctgtccttcctggattcctgtaacagcctcacgcccaagagcacacctgttaagaccctgcccttctcgccctcccagtttctgaacttctggaacaaacaggacacattggagctggagagcccctcgctgacatccaccccagtgtgcagccagaaggtggtggtcaccacaccactgcaccgggacaagacacccctgcaccagaaacatgctgcgtttgtaaccccagatcagaagtactccatggacaacactccccacacgccaaccccgttcaagaacgccctggagaagtacggacccctgaagcccctgccacagaccccgcacctggaggaggacttgaaggaggtgctgcgttctgaggctggcatcgaactcatcatcgaggacgacatcaggcccgagaagcagaagaggaagcctgggctgcggcggagccccatcaagaaagtccggaagtctctggctcttgacattgtggatgaggatgtgaagctgatgatgtccacactgcccaagtctctatccttgccgacaactgccccttcaaactcttccagcctcaccctgtcaggtatcaaagaagacaacagcttgctcaaccagggcttcttgcaggccaagcccgagaaggcagcagtggcccagaagccccgaagccacttcacgacacctgcccctatgtccagtgcctggaagacggtggcctgcggggggaccagggaccagcttttcatgcaggagaaagcccggcagctcctgggccgcctgaagcccagccacacatctcggaccctcatcttgtcctgaggtgttgagggtgtcacgagcccattctcatgtttacaggggttgtgggggcagagggggtctgtgaatctgagagtcattcaggtgacctcctgcagggagccttctgccaccagcccctccccagactctcaggtggaggcaacagggccatgtgctgccctgttgccgagcccagctgtgggcggctcctggtgctaacaacaaagttccacttccaggtctgcctggttccctccccaaggccacagggagctccgtcagcttctcccaagcccacgtcaggcctggcctcatctcagaccctgcttaggatgggggatgtggccaggggtgctcctgtgctcaccctctcttggtgcatttttttggaagaataaaattgcctctctctttgtgctggtctggaaaaaaaaa

SEQ ID NO: 65 - DNA, 12507 bp, Homo sapiens
taccgggcggaggtgagcgcggcgccggctcctcctgcggcggactttgggtgcgacttgacgagcggtggttcgacaagtggccttgcgggccggatcgtcccagtggaagagttgtaaatttgcttctggccttcccctacggattatacctggccttcccctacggattatactcaacttactgtttagaaaatgtggcccacgagacgcctggttactatcaaaaggagcggggtcgacggtccccactttcccctgagcctcagcacctgcttgtttggaaggggtattgaatgtgacatccgtatccagcttcctgttgtgtcaaaacaacattgcaaaattgaaatccatgagcaggaggcaatattacataatttcagttccacaaatccaacacaagtaaatgggtctgttattgatgagcctgtacggctaaaacatggagatgtaataactattattgatcgttccttcaggtatgaaaatgaaagtcttcagaatggaaggaagtcaactgaatttccaagaaaaatacgtgaacaggagccagcacgtcgtgtctcaagatctagcttctcttctgaccctgatgagaaagctcaagattccaaggcctattcaaaaatcactgaaggaaaagtttcaggaaatcctcaggtacatatcaagaatgtcaaagaagacagtaccgcagatgactcaaaagacagtgttgctcagggaacaactaatgttcattcctcagaacatgctggacgtaatggcagaaatgcagctgatcccatttctggggattttaaagaaatttccagcgttaaattagtgagccgttatggagaattgaagtctgttcccactacacaatgtcttgacaatagcaaaaaaaatgaatctcccttttggaagctttatgagtcagtgaagaaagagttggatgtaaaatcacaaaaagaaaatgtcctacagtattgtagaaaatctggattacaaactgattacgcaacagagaaagaaagtgctgatggtttacagggggagacccaactgttggtctcgcgtaagtcaagaccaaaatctggtgggagcggccacgctgtggcagagcctgcttcacctgaacaagagcttgaccagaacaaggggaagggaagagacgtggagtctgttcagactcccagcaaggctgtgggcgccagctttcctctctatgagccggctaaaatgaagacccctgtacaatattcacagcaacaaaattctccacaaaaacataagaacaaagacctgtatactactggtagaagagaatctgtgaatctgggtaaaagtgaaggcttcaaggctggtgataaaactcttactcccaggaagctttcaactagaaatcgaacaccagctaaagttgaagatgcagctgactctgccactaagccagaaaatctctcttccaaaaccagaggaagtattcctacagatgtggaagttctgcctacggaaactgaaattcacaatgagccatttttaactctgtggctcactcaagttgagaggaagatccaaaaggattccctcagcaagcctgagaaattgggcactacagctggacagatgtgctctgggttacctggtcttagttcagttgatatcaacaactttggtgattccattaatgagagtgagggaatacctttgaaaagaaggcgtgtgtcctttggtgggcacctaagacctgaactatttgatgaaaacttgcctcctaatacgcctctcaaaaggggagaagccccaaccaaaagaaagtctctggtaatgcacactccacctgtcctgaagaaaatcatcaaggaacagcctcaaccatcaggaaaacaagagtcaggttcagaaatccatgtggaagtgaaggcacaaagcttggttataagccctccagctcctagtcctaggaaaactccagttgccagtgatcaacgccgtaggtcctgcaaaacagcccctgcttccagcagcaaatctcagacagaggttcctaagagaggagggagaaagagtggcaacctgccttcaaagagagtgtctatcagccgaagtcaacatgatattttacagatgatatgttccaaaagaagaagtggtgcttcggaagcaaatctgattgttgcaaaatcatgggcagatgtagtaaaacttggtgcaaaacaaacacaaactaaagtcataaaacatggtcctcaaaggtcaatgaacaaaaggcaaagaagacctgctactccaaagaagcctgtgggcgaagttcacagtcaatttagtacaggccacgcaaactctccttgtaccataataatagggaaagctcatactgaaaaagtacatgtgcctgctcgaccctacagagtgctcaacaacttcatttccaaccaaaaaatggactttaaggaagatctttcaggaatagctgaaatgttcaagaccccagtgaaggagcaaccgcagttgacaagcacatgtcacatcgctatttcaaattcagagaatttgcttggaaaacagtttcaaggaactgattcaggagaagaacctctgctccccacctcagagagttttggaggaaatgtgttcttcagtgcacagaatgcagcaaaacagccatctgataaatgctctgcaagccctcccttaagacggcagtgtattagagaaaatggaaacgtagcaaaaacgcccaggaacacctacaaaatgacttctctggagacaaaaacttcagatactgagacagagccttcaaaaacagtatccactgcaaacaggtcaggaaggtctacagagttcaggaatatacagaagctacctgtggaaagtaagagtgaagaaacaaatacagaaattgttgagtgcatcctaaaaagaggtcagaaggcaacactactacaacaaaggagagaaggagagatgaaggaaatagaaagaccttttgagacatataaggaaaatattgaattaaaagaaaacgatgaaaagatgaaagcaatgaagagatcaagaacttgggggcagaaatgtgcaccaatgtctgacctgacagacctcaagagcttgcctgatacagaactcatgaaagacacggcacgtggccagaatctcctccaaacccaagatcatgccaaggcaccaaagagtgagaaaggcaaaatcactaaaatgccctgccagtcattacaaccagaaccaataaacaccccaacacacacaaaacaacagttgaaggcatccctggggaaagtaggtgtgaaagaagagctcctagcagtcggcaagttcacacggacgtcaggggagaccacgcacacgcacagagagccagcaggagatggcaagagcatcagaacgtttaaggagtctccaaagcagatcctggacccagcagcccgtgtaactggaatgaagaagtggccaagaacgcctaaggaagaggcccagtcactagaagacctggctggcttcaaagagctcttccagacaccaggtccctctgaggaatcaatgactgatgagaaaactaccaaaatagcctgcaaatctccaccaccagaatcagtggacactccaacaagcacaaagcaatggcctaagagaagtctcaggaaagcagatgtagaggaagaattcttagcactcaggaaactaacaccatcagcagggaaagccatgcttacgcccaaaccagcaggaggtgatgagaaagacattaaagcatttatgggaactccagtgcagaaactggacctggcaggaactttacctggcagcaaaagacagctacagactcctaaggaaaaggcccaggctctagaagacctggctggctttaaagagctcttccagactcctggtcacaccgaggaattagtggctgctggtaaaaccactaaaataccctgcgactctccacagtcagacccagtggacaccccaacaagcacaaagcaacgacccaagagaagtatcaggaaagcagatgtagagggagaactcttagcgtgcaggaatctaatgccatcagcaggcaaagccatgcacacgcctaaaccatcagtaggtgaagagaaagacatcatcatatttgtgggaactccagtgcagaaactggacctgacagagaacttaaccggcagcaagagacggccacaaactcctaaggaagaggcccaggctctggaagacctgactggctttaaagagctcttccagacccctggtcatactgaagaagcagtggctgctggcaaaactactaaaatgccctgcgaatcttctccaccagaatcagcagacaccccaacaagcacaagaaggcagcccaagacacctttggagaaaagggacgtacagaaggagctctcagccctgaagaagctcacacagacatcaggggaaaccacacacacagataaagtaccaggaggtgaggataaaagcatcaacgcgtttagggaaactgcaaaacagaaactggacccagcagcaagtgtaactggtagcaagaggcacccaaaaactaaggaaaaggcccaacccctagaagacctggctggcttgaaagagctcttccagacaccagtatgcactgacaagcccacgactcacgagaaaactaccaaaatagcctgcagatcacaaccagacccagtggacacaccaacaagctccaagccacagtccaagagaagtctcaggaaagtggacgtagaagaagaattcttcgcactcaggaaacgaacaccatcagcaggcaaagccatgcacacacccaaaccagcagtaagtggtgagaaaaacatctacgcatttatgggaactccagtgcagaaactggacctgacagagaacttaactggcagcaagagacggctacaaactcctaaggaaaaggcccaggctctagaagacctggctggctttaaagagctcttccagacacgaggtcacactgaggaatcaatgactaacgataaaactgccaaagtagcctgcaaatcttcacaaccagacccagacaaaaacccagcaagctccaagcgacggctcaagacatccctggggaaagtgggcgtgaaagaagagctcctagcagttggcaagctcacacagacatcaggagagactacacacacacacacagagccaacaggagatggtaagagcatgaaagcatttatggagtctccaaagcagatcttagactcagcagcaagtctaactggcagcaagaggcagctgagaactcctaagggaaagtctgaagtccctgaagacctggccggcttcatcgagctcttccagacaccaagtcacactaaggaatcaatgactaacgaaaaaactaccaaagtatcctacagagcttcacagccagacctagtggacaccccaacaagctccaagccacagcccaagagaagtctcaggaaagcagacactgaagaagaatttttagcatttaggaaacaaacgccatcagcaggcaaagccatgcacacacccaaaccagcagtaggtgaagagaaagacatcaacacgtttttgggaactccagtgcagaaactggaccagccaggaaatttacctggcagcaatagacggctacaaactcgtaaggaaaaggcccaggctctagaagaactgactggcttcagagagcttttccagacaccatgcactgataaccccacgactgatgagaaaactaccaaaaaaatactctgcaaatctccgcaatcagacccagcggacaccccaacaaacacaaagcaacggcccaagagaagcctcaagaaagcagacgtagaggaagaatttttagcattcaggaaactaacaccatcagcaggcaaagccatgcacacgcctaaagcagcagtaggtgaagagaaagacatcaacacatttgtggggactccagtggagaaactggacctgctaggaaatttacctggcagcaagagacggccacaaactcctaaagaaaaggccaaggctctagaagatctggctggcttcaaagagctcttccagacaccaggtcacactgaggaatcaatgaccgatgacaaaatcacagaagtatcctgcaaatctccacaaccagacccagtcaaaaccccaacaagctccaagcaacgactcaagatatccttggggaaagtaggtgtgaaagaagaggtcctaccagtcggcaagctcacacagacgtcagggaagaccacacagacacacagagagacagcaggagatggaaagagcatcaaagcgtttaaggaatctgcaaagcagatgctggacccagcaaactatggaactgggatggagaggtggccaagaacacctaaggaagaggcccaatcactagaagacctggccggcttcaaagagctcttccagacaccagaccacactgaggaatcaacaactgatgacaaaactaccaaaatagcctgcaaatctccaccaccagaatcaatggacactccaacaagcacaaggaggcggcccaaaacacctttggggaaaagggatatagtggaagagctctcagccctgaagcagctcacacagaccacacacacagacaaagtaccaggagatgaggataaaggcatcaacgtgttcagggaaactgcaaaacagaaactggacccagcagcaagtgtaactggtagcaagaggcagccaagaactcctaagggaaaagcccaacccctagaagacttggctggcttgaaagagctcttccagacaccaatatgcactgacaagcccacgactcatgagaaaactaccaaaatagcctgcagatctccacaaccagacccagtgggtaccccaacaatcttcaagccacagtccaagagaagtctcaggaaagcagacgtagaggaagaatccttagcactcaggaaacgaacaccatcagtagggaaagctatggacacacccaaaccagcaggaggtgatgagaaagacatgaaagcatttatgggaactccagtgcagaaattggacctgccaggaaatttacctggcagcaaaagatggccacaaactcctaaggaaaaggcccaggctctagaagacctggctggcttcaaagagctcttccagacaccaggcactgacaagcccacgactgatgagaaaactaccaaaatagcctgcaaatctccacaaccagacccagtggacaccccagcaagcacaaagcaacggcccaagagaaacctcaggaaagcagacgtagaggaagaatttttagcactcaggaaacgaacaccatcagcaggcaaagccatggacacaccaaaaccagcagtaagtgatgagaaaaatatcaacacatttgtggaaactccagtgcagaaactggacctgctaggaaatttacctggcagcaagagacagccacagactcctaaggaaaaggctgaggctctagaggacctggttggcttcaaagaactcttccagacaccaggtcacactgaggaatcaatgactgatgacaaaatcacagaagtatcctgtaaatctccacagccagagtcattcaaaacctcaagaagctccaagcaaaggctcaagatacccctggtgaaagtggacatgaaagaagagcccctagcagtcagcaagctcacacggacatcaggggagactacgcaaacacacacagagccaacaggagatagtaagagcatcaaagcgtttaaggagtctccaaagcagatcctggacccagcagcaagtgtaactggtagcaggaggcagctgagaactcgtaaggaaaaggcccgtgctctagaagacctggttgacttcaaagagctcttctcagcaccaggtcacactgaagagtcaatgactattgacaaaaacacaaaaattccctgcaaatctcccccaccagaactaacagacactgccacgagcacaaagagatgccccaagacacgtcccaggaaagaagtaaaagaggagctctcagcagttgagaggctcacgcaaacatcagggcaaagcacacacacacacaaagaaccagcaagcggtgatgagggcatcaaagtattgaagcaacgtgcaaagaagaaaccaaacccagtagaagaggaacccagcaggagaaggccaagagcacctaaggaaaaggcccaacccctggaagacctggccggcttcacagagctctctgaaacatcaggtcacactcaggaatcactgactgctggcaaagccactaaaataccctgcgaatctcccccactagaagtggtagacaccacagcaagcacaaagaggcatctcaggacacgtgtgcagaaggtacaagtaaaagaagagccttcagcagtcaagttcacacaaacatcaggggaaaccacggatgcagacaaagaaccagcaggtgaagataaaggcatcaaagcattgaaggaatctgcaaaacagacaccggctccagcagcaagtgtaactggcagcaggagacggccaagagcacccagggaaagtgcccaagccatagaagacctagctggcttcaaagacccagcagcaggtcacactgaagaatcaatgactgatgacaaaaccactaaaataccctgcaaatcatcaccagaactagaagacaccgcaacaagctcaaagagacggcccaggacacgtgcccagaaagtagaagtgaaggaggagctgttagcagttggcaagctcacacaaacctcaggggagaccacgcacaccgacaaagagccggtaggtgagggcaaaggcacgaaagcatttaagcaacctgcaaagcggaagctggacgcagaagatgtaattggcagcaggagacagccaagagcacctaaggaaaaggcccaacccctggaagatctggccagcttccaagagctctctcaaacaccaggccacactgaggaactggcaaatggtgctgctgatagctttacaagcgctccaaagcaaacacctgacagtggaaaacctctaaaaatatccagaagagttcttcgggcccctaaagtagaacccgtgggagacgtggtaagcaccagagaccctgtaaaatcacaaagcaaaagcaacacttccctgcccccactgcccttcaagaggggaggtggcaaagatggaagcgtcacgggaaccaagaggctgcgctgcatgccagcaccagaggaaattgtggaggagctgccagccagcaagaagcagagggttgctcccagggcaagaggcaaatcatccgaacccgtggtcatcatgaagagaagtttgaggacttctgcaaaaagaattgaacctgcggaagagctgaacagcaacgacatgaaaaccaacaaagaggaacacaaattacaagactcggtccctgaaaataagggaatatccctgcgctccagacgccaaaataagactgaggcagaacagcaaataactgaggtctttgtattagcagaaagaatagaaataaacagaaatgaaaagaagcccatgaagacctccccagagatggacattcagaatccagatgatggagcccggaaacccatacctagagacaaagtcactgagaacaaaaggtgcttgaggtctgctagacagaatgagagctcccagcctaaggtggcagaggagagcggagggcagaagagtgcgaaggttctcatgcagaatcagaaagggaaaggagaagcaggaaattcagactccatgtgcctgagatcaagaaagacaaaaagccagcctgcagcaagcactttggagagcaaatctgtgcagagagtaacgcggagtgtcaagaggtgtgcagaaaatccaaagaaggctgaggacaatgtgtgtgtcaagaaaataagaaccagaagtcatagggacagtgaagatatttgacagaaaaatcgaactgggaaaaatataataaagttagttttgtgataagttctagtgcagtttttgtcataaattacaagtgaattctgtaagtaaggctgtcagtctgcttaagggaagaaaactttggatttgctgggtctgaatcggcttcataaactccactgggagcactgctgggctcctggactgagaatagttgaacaccgggggctttgtgaaggagtctgggccaaggtttgccctcagctttgcagaatgaagccttgaggtctgtcaccacccacagccaccctacagcagccttaactgtgacacttgccacactgtgtcgtcgtttgtttgcctatgtcctccagggcacggtggcaggaacaactatcctcgtctgtcccaacactgagcaggcactcggtaaacacgaatgaatggatgagcgcacggatgaatggagcttacaagatctgtctttccaatggccgggggcatttggtccccaaattaaggctattggacatctgcacaggacagtcctatttttgatgtcctttcctttctgaaaataaagttttgtgctttggagaatgactcgtgagcacatctttagggaccaagagtgactttctgtaaggagtgactcgtggcttgccttggtctcttgggaatacttttctaactagggttgctctcacctgagacattctccacccgcggaatctcagggtcccaggctgtgggccatcacgacctcaaactggctcctaatctccagctttcctgtcattgaaagcttcggaagtttactggctctgctcccgcctgttttctttctgactctatctggcagcccgatgccacccagtacaggaagtgacaccagtactctgtaaagcatcatcatccttggagagactgagcactcagcaccttcagccacgatttcaggatcgcttccttgtgagccgctgcctccgaaatctcctttgaagcccagacatctttctccagcttcagacttgtagatataactcgttcatcttcatttactttccactttgccccctgtcctctctgtgttccccaaatcagagaatagcccgccatcccccaggtcacctgtctggattcctccccattcacccaccttgccaggtgcaggtgaggatggtgcaccagacagggtagctgtcccccaaaatgtgccctgtgcgggcagtgccctgtctccacgtttgtttccccagtgtctggcggggagccaggtgacatcataaatacttgctgaatgaatgcagaaatcagcggtactgacttgtactatattggctgccatgatagggttctcacagcgtcatccatgatcgtaagggagaatgacattctgcttgagggagggaatagaaaggggcagggaggggacatctgagggcttcacagggctgcaaagggtacagggattgcaccagggcagaacaggggagggtgttcaaggaagagtggctcttagcagaggcactttggaaggtgtgaggcataaatgcttccttctacgtaggccaacctcaaaactttcagtaggaatgttgctatgatcaagttgttctaacactttagacttagtagtaattatgaacctcacatagaaaaatttcatccagccatatgcctgtggagtggaatattctgtttagtagaaaaatcctttagagttcagctctaaccagaaatcttgctgaagtatgtcagcaccttttctcaccctggtaagtacagtatttcaagagcacgctaagggtggttttcattttacagggctgttgatgatgggttaaaaatgttcatttaagggctacccccgtgtttaatagatgaacaccacttctacacaaccctccttggtactgggggagggagagatctgacaaatactgcccattcccctaggctgactggatttgagaacaaatacccacccatttccaccatggtatggtaacttctctgagcttcagtttccaagtgaatttccatgtaataggacattcccattaaatacaagctgtttttactttttcgcctcccagggcctgtgggatctggtcccccagcctctcttgggctttcttacactaactctgtacctaccatctcctgcctcccttaggcaggcacctccaaccaccacacactccctgctgttttccctgcctggaactttccctcctgccccaccaagatcatttcatccagtcctgagctcagcttaagggaggcttcttgcctgtgggttccctcacccccatgcctgtcctccaggctggggcaggttcttagtttgcctggaattgttctgtacctctttgtagcacgtagtgttgtggaaactaagccactaattgagtttctggctcccctcctggggttgtaagttttgttcattcatgagggccgactgcatttcctggttactctatcccagtgaccagccacaggagatgtccaataaagtatgtgatgaaatggtcttaaaaaaaaaaaaaa

SEQ ID NO: 66 - DNA, 1697 bp, Homo sapiens
gaggcgtaagccaggcgtgttaaagccggtcggaactgctccggagggcacgggctccgtaggcaccaactgcaaggacccctccccctgcgggcgctcccatggcacagttcgcgttcgagagtgacctgcactcgctgcttcagctggatgcacccatccccaatgcaccccctgcgcgctggcagcgcaaagccaaggaagccgcaggcccggccccctcacccatgcgggccgccaaccgatcccacagcgccggcaggactccgggccgaactcctggcaaatccagttccaaggttcagaccactcctagcaaacctggcggtgaccgctatatcccccatcgcagtgctgcccagatggaggtggccagcttcctcctgagcaaggagaaccagcctgaaaacagccagacgcccaccaagaaggaacatcagaaagcctgggctttgaacctgaacggttttgatgtagaggaagccaagatccttcggctcagtggaaaaccacaaaatgcgccagagggttatcagaacagactgaaagtactctacagccaaaaggccactcctggctccagccggaagacctgccgttacattccttccctgccagaccgtatcctggatgcgcctgaaatccgaaatgactattacctgaaccttgtggattggagttctgggaatgtactggccgtggcactggacaacagtgtgtacctgtggagtgcaagctctggtgacatcctgcagcttttgcaaatggagcagcctggggaatatatatcctctgtggcctggatcaaagagggcaactacttggctgtgggcaccagcagtgctgaggtgcagctatgggatgtgcagcagcagaaacggcttcgaaatatgaccagtcactctgcccgagtgggctccctaagctggaacagctatatcctgtccagtggttcacgttctggccacatccaccaccatgatgttcgggtagcagaacaccatgtggccacactgagtggccacagccaggaagtgtgtgggctgcgctgggccccagatggacgacatttggccagtggtggtaatgataacttggtcaatgtgtggcctagtgctcctggagagggtggctgggttcctctgcagacattcacccagcatcaaggggctgtcaaggccgtagcatggtgtccctggcagtccaatgtcctggcaacaggagggggcaccagtgatcgacacattcgcatctggaatgtgtgctctggggcctgtctgagtgccgtggatgcccattcccaggtgtgctccatcctctggtctccccattacaaggagctcatctcaggccatggctttgcacagaaccagctagttatttggaagtacccaaccatggccaaggtggctgaactcaaaggtcacacatcccgggtcctgagtctgaccatgagcccagatggggccacagtggcatccgcagcagcagatgagaccctgaggctatggcgctgttttgagttggaccctgcgcggcggcgggagcgggagaaggccagtgcagccaaaagcagcctcatccaccaaggcatccgctgaagaccaacccatcacctcagttgttttttatttttctaataaagtcatgtctcccttcatgttttttttttaaaaaaaaaaaaaaaaaaaaaaaaa

SEQ ID NO: 67 - DNA, 2656 bp, Homo sapiens; identical to SEQ ID NO: 9

SEQ ID NO: 68 - DNA, 5753 bp, Homo sapiens
gattggctggtctgcttcgggcgggctaaaggaaggttcaagtggagctctcctaaccgacgcgcgtctgtggagaagcggcttggtcgggggtggtctcgtggggtcctgcctgtttagtcgctttcagggttcttgagccccttcacgaccgtcaccatggaagtgtcaccattgcagcctgtaaatgaaaatatgcaagtcaacaaaataaagaaaaatgaagatgctaagaaaagactgtctgttgaaagaatctatcaaaagaaaacacaattggaacatattttgctccgcccagacacctacattggttctgtggaattagtgacccagcaaatgtgggtttacgatgaagatgttggcattaactatagggaagtcacttttgttcctggtttgtacaaaatctttgatgagattctagttaatgctgcggacaacaaacaaagggacccaaaaatgtcttgtattagagtcacaattgatccggaaaacaatttaattagtatatggaataatggaaaaggtattcctgttgttgaacacaaagttgaaaagatgtatgtcccagctctcatatttggacagctcctaacttctagtaactatgatgatgatgaaaagaaagtgacaggtggtcgaaatggctatggagccaaattgtgtaacatattcagtaccaaatttactgtggaaacagccagtagagaatacaagaaaatgttcaaacagacatggatggataatatgggaagagctggtgagatggaactcaagcccttcaatggagaagattatacatgtatcacctttcagcctgatttgtctaagtttaaaatgcaaagcctggacaaagatattgttgcactaatggtcagaagagcatatgatattgctggatccaccaaagatgtcaaagtctttcttaatggaaataaactgccagtaaaaggatttcgtagttatgtggacatgtatttgaaggacaagttggatgaaactggtaactccttgaaagtaatacatgaacaagtaaaccacaggtgggaagtgtgtttaactatgagtgaaaaaggctttcagcaaattagctttgtcaacagcattgctacatccaagggtggcagacatgttgattatgtagctgatcagattgtgactaaacttgttgatgttgtgaagaagaagaacaagggtggtgttgcagtaaaagcacatcaggtgaaaaatcacatgtggatttttgtaaatgccttaattgaaaacccaacctttgactctcagacaaaagaaaacatgactttacaacccaagagctttggatcaacatgccaattgagtgaaaaatttatcaaagctgccattggctgtggtattgtagaaagcatactaaactgggtgaagtttaaggcccaagtccagttaaacaagaagtgttcagctgtaaaacataatagaatcaagggaattcccaaactcgatgatgccaatgatgcagggggccgaaactccactgagtgtacgcttatcctgactgagggagattcagccaaaactttggctgtttcaggccttggtgtggttgggagagacaaatatggggttttccctcttagaggaaaaatactcaatgttcgagaagcttctcataagcagatcatggaaaatgctgagattaacaatatcatcaagattgtgggtcttcagtacaagaaaaactatgaagatgaagattcattgaagacgcttcgttatgggaagataatgattatgacagatcaggaccaagatggttcccacatcaaaggcttgctgattaattttatccatcacaactggccctctcttctgcgacatcgttttctggaggaatttatcactcccattgtaaaggtatctaaaaacaagcaagaaatggcattttacagccttcctgaatttgaagagtggaagagttctactccaaatcataaaaaatggaaagtcaaatattacaaaggtttgggcaccagcacatcaaaggaagctaaagaatactttgcagatatgaaaagacatcgtatccagttcaaatattctggtcctgaagatgatgctgctatcagcctggcctttagcaaaaaacagatagatgatcgaaaggaatggttaactaatttcatggaggatagaagacaacgaaagttacttgggcttcctgaggattacttgtatggacaaactaccacatatctgacatataatgacttcatcaacaaggaacttatcttgttctcaaattctgataacgagagatctatcccttctatggtggatggtttgaaaccaggtcagagaaaggttttgtttacttgcttcaaacggaatgacaagcgagaagtaaaggttgcccaattagctggatcagtggctgaaatgtcttcttatcatcatggtgagatgtcactaatgatgaccattatcaatttggctcagaattttgtgggtagcaataatctaaacctcttgcagcccattggtcagtttggtaccaggctacatggtggcaaggattctgctagtccacgatacatctttacaatgctcagctctttggctcgattgttatttccaccaaaagatgatcacacgttgaagtttttatatgatgacaaccagcgtgttgagcctgaatggtacattcctattattcccatggtgctgataaatggtgctgaaggaatcggtactgggtggtcctgcaaaatccccaactttgatgtgcgtgaaattgtaaataacatcaggcgtttgatggatggagaagaacctttgccaatgcttccaagttacaagaacttcaagggtactattgaagaactggctccaaatcaatatgtgattagtggtgaagtagctattcttaattctacaaccattgaaatctcagagcttcccgtcagaacatggacccagacatacaaagaacaagttctagaacccatgttgaatggcaccgagaagacacctcctctcataacagactatagggaataccatacagataccactgtgaaatttgttgtgaagatgactgaagaaaaactggcagaggcagagagagttggactacacaaagtcttcaaactccaaactagtctcacatgcaactctatggtgctttttgaccacgtaggctgtttaaagaaatatgacacggtgttggatattctaagagacttttttgaactcagacttaaatattatggattaagaaaagaatggctcctaggaatgcttggtgctgaatctgctaaactgaataatcaggctcgctttatcttagagaaaatagatggcaaaataatcattgaaaataagcctaagaaagaattaattaaagttctgattcagaggggatatgattcggatcctgtgaaggcctggaaagaagcccagcaaaaggttccagatgaagaagaaaatgaagagagtgacaacgaaaaggaaactgaaaagagtgactccgtaacagattctggaccaaccttcaactatcttcttgatatgcccctttggtatttaaccaaggaaaagaaagatgaactctgcaggctaagaaatgaaaaagaacaagagctggacacattaaaaagaaagagtccatcagatttgtggaaagaagacttggctacatttattgaagaattggaggctgttgaagccaaggaaaaacaagatgaacaagtcggacttcctgggaaaggggggaaggccaaggggaaaaaaacacaaatggctgaagttttgccttctccgcgtggtcaaagagtcattccacgaataaccatagaaatgaaagcagaggcagaaaagaaaaataaaaagaaaattaagaatgaaaatactgaaggaagccctcaagaagatggtgtggaactagaaggcctaaaacaaagattagaaaagaaacagaaaagagaaccaggtacaaagacaaagaaacaaactacattggcatttaagccaatcaaaaaaggaaagaagagaaatccctggtctgattcagaatcagataggagcagtgacgaaagtaattttgatgtccctccacgagaaacagagccacggagagcagcaacaaaaacaaaattcacaatggatttggattcagatgaagatttctcagattttgatgaaaaaactgatgatgaagattttgtcccatcagatgctagtccacctaagaccaaaacttccccaaaacttagtaacaaagaactgaaaccacagaaaagtgtcgtgtcagaccttgaagctgatgatgttaagggcagtgtaccactgtcttcaagccctcctgctacacatttcccagatgaaactgaaattacaaacccagttcctaaaaagaatgtgacagtgaagaagacagcagcaaaaagtcagtcttccacctccactaccggtgccaaaaaaagggctgccccaaaaggaactaaaagggatccagctttgaattctggtgtctctcaaaagcctgatcctgccaaaaccaagaatcgccgcaaaaggaagccatccacttctgatgattctgactctaattttgagaaaattgtttcgaaagcagtcacaagcaagaaatccaagggggagagtgatgacttccatatggactttgactcagctgtggctcctcgggcaaaatctgtacgggcaaagaaacctataaagtacctggaagagtcagatgaagatgatctgttttaaaatgtgaggcgattattttaagtaattatcttaccaagcccaagactggttttaaagttacctgaagctcttaacttcctcccctctgaatttagtttggggaaggtgtttttagtacaagacatcaaagtgaagtaaagcccaagtgttctttagctttttataatactgtctaaatagtgaccatctcatgggcattgttttcttctctgctttgtctgtgttttgagtctgctttcttttgtctttaaaacctgatttttaagttcttctgaactgtagaaatagctatctgatcacttcagcgtaaagcagtgtgtttattaaccatccactaagctaaaactagagcagtttgatttaaaagtgtcactcttcctccttttctactttcagtagatatgagatagagcataattatctgttttatcttagttttatacataatttaccatcagatagaactttatggttctagtacagatactctactacactcagcctcttatgtgccaagtttttctttaagcaatgagaaattgctcatgttcttcatcttctcaaatcatcagaggccgaagaaaaacactttggctgtgtctataacttgacacagtcaatagaatgaagaaaattagagtagttatgtgattatttcagctcttgacctgtcccctctggctgcctctgagtctgaatctcccaaagagagaaaccaatttctaagaggactggattgcagaagactcggggacaacatttgatccaagatcttaaatgttatattgataaccatgctcagcaatgagctattagattcattttgggaaatctccataatttcaatttgtaaactttgttaagacctgtctacattgttatatgtgtgtgacttgagtaatgttatcaacgtttttgtaaatatttactatgtttttctattagctaaattccaacaattttgtactttaataaaatgttctaaacattgcaaccca

SEQ ID NO: 69 - DNA, 2655 bp, Homo sapiens
cccagaaggccgcggggggtggaccgcctaagagggcgtgcgctcccgacatgccccgcggcgcgccattaaccgccagatttgaatcgcgggacccgttggcagaggtggcggcggcggcatgggtgccccgacgttgccccctgcctggcagccctttctcaaggaccaccgcatctctacattcaagaactggcccttcttggagggctgcgcctgcaccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcggagaaagtgcgccgtgccatcgagcagctggctgccatggattgaggcctctggccggagctgcctggtcccagagtggctgcaccacttccagggtttattccctggtgccaccagccttcctgtgggccccttagcaatgtcttaggaaaggagatcaacattttcaaattagatgtttcaactgtgctcttgttttgtcttgaaagtggcaccagaggtgcttctgcctgtgcagcgggtgctgctggtaacagtggctgcttctctctctctctctcttttttgggggctcatttttgctgttttgattcccgggcttaccaggtgagaagtgagggaggaagaaggcagtgtcccttttgctagagctgacagctttgttcgcgtgggcagagccttccacagtgaatgtgtctggacctcatgttgttgaggctgtcacagtcctgagtgtggacttggcaggtgcctgttgaatctgagctgcaggttccttatctgtcacacctgtgcctcctcagaggacagtttttttgttgttgtgtttttttgtttttttttttttggtagatgcatgacttgtgtgtgatgagagaatggagacagagtccctggctcctctactgtttaacaacatggctttcttattttgtttgaattgttaattcacagaatagcacaaactacaattaaaactaagcacaaagccattctaagtcattggggaaacggggtgaacttcaggtggatgaggagacagaatagagtgataggaagcgtctggcagatactccttttgccactgctgtgtgattagacaggcccagtgagccgcggggcacatgctggccgctcctccctcagaaaaaggcagtggcctaaatcctttttaaatgacttggctcgatgctgtgggggactggctgggctgctgcaggccgtgtgtctgtcagcccaaccttcacatctgtcacgttctccacacgggggagagacgcagtccgcccaggtccccgctttctttggaggcagcagctcccgcagggctgaagtctggcgtaagatgatggatttgattcgccctcctccctgtcatagagctgcagggtggattgttacagcttcgctggaaacctctggaggtcatctcggctgttcctgagaaataaaaagcctgtcatttcaaacactgctgtggaccctactgggtttttaaaatattgtcagtttttcatcgtcgtccctagcctgccaacagccatctgcccagacagccgcagtgaggatgagcgtcctggcagagacgcagttgtctctgggcgcttgccagagccacgaaccccagacctgtttgtatcatccgggctccttccgggcagaaacaactgaaaatgcacttcagacccacttatttctgccacatctgagtcggcctgagatagacttttccctctaaactgggagaatatcacagtggtttttgttagcagaaaatgcactccagcctctgtactcatctaagctgcttatttttgatatttgtgtcagtctgtaaatggatacttcactttaataactgttgcttagtaattggctttgtagagaagctggaaaaaaatggttttgtcttcaactcctttgcatgccaggcggtgatgtggatctcggcttctgtgagcctgtgctgtgggcagggctgagctggagccgcccctctcagcccgcctgccacggcctttccttaaaggccatccttaaaaccagaccctcatggctaccagcacctgaaagcttcctcgacatctgttaataaagccgtaggcccttgtctaagtgcaaccgcctagactttctttcagatacatgtccacatgtccatttttcaggttctctaagttggagtggagtctgggaagggttgtgaatgaggcttctgggctatgggtgaggttccaatggcaggttagagcccctcgggccaactgccatcctggaaagtagagacagcagtgcccgctgcccagaagagaccagcaagccaaactggagcccccattgcaggctgtcgccatgtggaaagagtaactcacaattgccaataaagtctcatgtggttttatctaaaaaaaaaaaaaaaaaaaaaaaaa

SEQ ID NO: 70 - DNA, 10906 bp, Homo sapiens
attggtggaggcggcaagtttaaacagagtcaaaacgccatacttgtttggctcctctttttaatttgcgagtttattgggcttgttttctgttttctagggagtaggttagtggaaaagaaaaagggccgaattcactcccacgacctctacagccgcccctgaggggaagcggtcagcgtaagtcccggatccccgctccggagccgcctcgtgggagcggggcaaggagatccaggaggggtctcgaatctgccatggcgaaccggcgagtggggcgaggctgctgggaagtgagcccgaccgagcggaggccgcccgcggggctgcggggccccgcggccgaggaggaggcgtcttccccgccggtcctgtctctcagccacttctgcaggtctcctttcctttgcttcggggacgttctcctgggagcctcacggacgctgtctctggccctagacaaccctaacgaggaggtggcagaagtgaagatctcccacttcccggccgcggacctgggcttcagtgtgtcgcagcgctgtttcgtgttgcagcctaaagagaaaattgttatttctgttaactggacaccactcaaagaaggccgagtaagagagattatgacatttcttgtaaatgatgttctgaaacaccaagctatattactaggaaatgcagaagagcagaaaaagaaaaagaggagtctttgggataccattaaaaagaagaaaatttcagcctctacaagtcacaacagaagggtttcaaatattcagaatgttaataaaacatttagtgtttcccaaaaagttgacagagttaggagcccactacaagcttgtgaaaacttggctatgaatgaaggcggtcccccaacagaaaacaattctttaatacttgaagaaaataaaatacccatatcacctattagccctgctttcaatgaatgccatggtgcaacttgcttgccactctctgtacgtcgatctactacctactcatctcttcatgcatcagaaaatagggaactattaaatgtacacagtgccaacgtttcaaaagtttcttttaatgagaaagctgtaactgaaacttcctttaattccgtaaatgttaatggccaaagaggagagaatagtaaacttagtcttacccccaactgttcttcaactttgaacattacacaaagccaaatacattttctaagtccagattcttttgtaaataatagtcatggagctaataatgaactagaattagtaacatgtctttcatcagatatgtttatgaaagataattcacagcctgtgcatttggaatcaacaattgcacatgaaatttatcagaaaattttaagtccagattctttcataaaagataattatggactaaatcaggatctagaatcagagtcagttaatcctattttatcccctaatcaatttttaaaagataacatggcatatatgtgtacatctcagcaaacatgtaaagtaccattatcaaatgaaaattctcaagtcccacagtctcctgaagattggagaaaaagtgaagtttcgccacgtattcctgaatgtcagggttcaaaatctcccaaagctatttttgaagaactagtagaaatgaagtcaaattactacagttttataaaacaaaataatcctaaattttctgcagttcaggatatttctagtcatagccacaataaacaacctaagagacgtccaatactttctgccactgttactaaaaggaaggccacctgtaccagagaaaaccaaactgagattaataaaccaaaagcaaaaagatgtctcaacagtgcagtgggtgaacatgaaaaagtaataaataatcaaaaggaaaaagaagattttcattcttatcttccaattatagatccaatattaagtaaatctaagagttataaaaacgaggtaacaccctcttcgacaacagcttcagttgctcggaaaagaaagagcgatggaagcatggaagatgcaaatgtgagagttgcaattacagaacatacagaagtgcgagaaatcaaaagaatccatttttctccctcagagcctaaaacatcagctgttaagaaaacaaaaaatgtgacaacacccatctcaaaacgtattagcaacagagagaaattaaacctgaagaagaaaactgatttatcaatattcagaactccaatttctaaaacaaacaaaaggacaaaacccattatcgctgtggcacagtccagtttgaccttcataaaaccattaaaaacagatattcccagacacccgatgccatttgctgcaaaaaacatgttttatgatgaacgctggaaggaaaagcaggaacagggcttcacttggtggttaaattttatattaacccctgatgacttcactgtaaaaacaaatatttctgaagtaaatgctgctactcttcttttgggaatagagaatcaacataaaataagtgttcctagagcacctacaaaagaggaaatgtctctcagagcttatactgctcggtgtaggttaaacagactacgtcgtgcagcatgccgtttgtttacttctgaaaaaatggttaaagctattaaaaagcttgaaattgaaattgaagctaggcggttaattgttcgaaaagatagacacctatggaaagatgtgggagaacgtcagaaagtcctgaattggctgttgtcctacaatcctttgtggcttcgaattggtctagagacaacttatggagaactcatatctttggaagataacagtgatgtcacagggttggctatgtttattctgaatcgcctactttggaatcctgatatagcagctgagtatagacaccccactgttcctcacctgtatagagatggtcatgaagaagctttgtccaagtttacattgaaaaagttattgttgttggtctgttttcttgattatgctaaaatttccagactcattgatcatgatccttgtctcttctgtaaagatgccgaattcaaggctagtaaagaaatccttttggctttttcacgagatttcctaagtggtgaaggtgacctttcccgtcaccttggcttattgggattacctgttaaccatgttcagacaccatttgatgaatttgattttgccgttacaaatcttgccgtagacttgcaatgtggagtgcgccttgtgcgaaccatggaacttctcacacagaactgggacctctcaaagaaactcaggattccggcaataagtcgtcttcaaaagatgcacaatgttgacattgttcttcaagttcttaaatcacgaggaattgaattaagtgatgagcatggaaatacaattctatctaaggatattgtggataggcacagagaaaaaactctcaggttgctttggaaaatagcgtttgcttttcaggtggatatttcccttaacttagatcaattaaaggaagaaattgcctttctaaaacacacaaagagtataaagaaaacaatatctctactatcatgccattctgatgatcttattaataagaaaaaaggcaaaagggatagtggttcctttgaacaatatagtgaaaacataaagttattgatggattgggtaaatgctgtttgtgccttctataataaaaaggtggagaattttacagtgtctttctcagacggccgtgtgttatgttacctgatccaccattaccatccttgctatgtgccatttgacgctatatgtcagcgtactactcaaactgtggaatgtacgcaaactggttcagtggtattaaattcatcatctgaatctgatgacagttctctggatatgtctcttaaagcatttgatcatgaaaatacttcagagctatacaaagagctcctagaaaatgaaaagaaaaattttcacttggttaggtctgcagttagagaccttggtggaatacctgctatgattaatcattcagatatgtcaaatacaattccagatgaaaaggtggttattacctatttgtcatttctttgtgcaaggcttttggatcttcgtaaagaaataagagctgctcgactcatacaaacaacatggagaaaatataaactaaaaacagatctcaaacgccatcaggagagagagaaagctgcaagaattattcaattggctgtaatcaattttctagcaaaacaaagattgagaaaaagagttaatgcagcactcgtcattcagaaatattggcgaagagtcttagcacagagaaaattattaatgttaaaaaaggaaaagctggaaaaagttcaaaataaagcagcatcacttattcagggatattggagaagatattccactagacaaagatttctgaaattgaaatattattcaatcatcctgcaatctaggataagaatgataattgctgttacatcttataaacgatatctttgggctacagttacaattcagaggcattggcgtgcttatttaagaagaaaacaagatcaacaaagatatgaaatgctaaaatcatcaactcttataatccaatctatgttcagaaaatggaagcaacgtaaaatgcaatcacaagtaaaagctacagtaatattgcaaagagcttttagagaatggcatttaagaaaacaagctaaagaagaaaattctgctattatcatacaatcatggtatagaatgcataaagaattacggaaatatatttatattagatcttgtgttgttatcattcagaaaagatttcggtgctttcaagcccaaaagttatataaaagaagaaaagagtccatactaaccatccagaagtactacaaagcatatctgaaaggaaagattgagcgcaccaactatttgcagaaacgagctgcagccattcaattacaagctgcttttaggagactgaaagctcataatttatgtagacaaattagagctgcttgtgttattcagtcatactggagaatgagacaagacagagttcgatttttaaaccttaagaagactattatcaaatttcaggcacatgtaagaaaacatcaacaacgacagaaatataagaagatgaagaaagcagctgttataattcagactcatttccgagcttatatttttgccatgaaagttctagcatcttaccagaaaacacgctctgctgtcattgtgctgcagtctgcatatagagggatgcaagccaggaaaatgtatattcacatcctcacatctgttataaagattcaatcatattatcgtgcttatgtttctaaaaaggaatttttgagcctaaaaaatgctacaataaaattgcagtcaactgttaagatgaaacaaacacgtaaacaatatttgcatttaagagcagctgcactatttatccagcaatgttaccgttccaaaaaaatagctgcacaaaagagagaagagtatatgcagatgcgggaatcttgtatcaaactgcaagcatttgttagaggataccttgtccgaaagcagatgaggttacaaagaaaagctgttatttcactacagtcttatttcagaatgagaaaggctcggcagtattatctgaaaatgtataaagcaattattgtcattcagaattactatcatgcatacaaagcacaggtcaatcagaggaagaacttcttgcaagtcaaaaaagcagctacttgcttgcaagcagcttacagaggttataaagtacgccagctaatcaaacaacaatctatagctgctcttaaaattcagtctgcttttagaggctataataaaagggtaaaatatcaatctgtgcttcaatctataataaagattcagagatggtacagggcgtacaagactcttcatgatacaagaacacattttttgaagacaaaggcagctgtgatttccctccagtctgcttatcgtggctggaaggttcggaaacagattagaagggaacatcaagctgccttgaagattcagtctgcttttagaatggccaaggcccagaaacagtttagattgtttaaaacagcagcattagtcatccagcaaaatttcagagcatggactgcaggaaggaagcaatgtatggagtatattgaactccgtcatgcggtactggtgcttcaatctatgtggaagggaaaaacactgagaagacagcttcaaaggcaacataaatgtgctatcatcatacagtcatactatagaatgcatgtgcaacaaaagaagtggaaaatcatgaaaaaagctgctcttctgattcaaaagtattatagggcttacagtattggaagagaacagaatcatttatatttgaaaacaaaagcagctgtagtaactttacagtcagcttatcgtggtatgaaagtgagaaaaagaataaaggattgcaacaaagcagcagtcactatacagtctaaatacagagcttacaaaaccaaaaagaaatatgcaacctatagagcttcagctattataattcagagatggtatcgaggtattaaaattacaaaccatcagcataaggagtatcttaatttgaagaagacagcaattaaaatccaatctgtttatagaggtattagagttagaagacatattcaacacatgcacagggcagccacttttattaaagccatgtttaaaatgcatcagtcaagaataagttaccatacaatgagaaaagcagctattgttattcaagtaagatgtagagcatattatcaaggtaaaatgcagcgtgaaaagtacctgacaattttgaaagctgttaaagtccttcaggcaagttttagaggagtaagagttagacggactcttagaaagatgcagactgcagcaacactcattcagtcaaactacagaagatacagacagcaaacatactttaataagttaaagaaaataacaaaaacagtacagcaaagatactgggcaatgaaagaaagaaacatacaatttcaaaggtataacaaactgaggcattctgtaatatacattcaggctatttttaggggaaagaaagctagaagacatttaaaaatgatgcatatagccgcaactctcattcagaggagatttagaactctaatgatgagaagaagattcctctctctcaagaaaactgctattttgattcagagaaaatatcgggcacatctttgtacaaagcatcacttacagttccttcaggtacaaaatgcagttattaaaatccagtcatcatacagaagatggatgataaggaaaaggatgcgagagatgcacagggctgctactttcatccagtctactttcagaatgcacagattacatatgagatatcaggctttgaaacaggcctccgttgtgatccaacagcaataccaagcaaatagagctgcaaaactgcagaggcagcattatctcagacaaagacactctgctgtgatccttcaggctgcattcaggggtatgaaaactagaagacatttgaagagtatgcattcctctgcaacccttattcagagtaggtttagatcattactggtgaggagaagattcatttccctcaaaaaagctactatttttgttcagaggaaatatcgagccaccatttgtgccaaacataaattgtaccaattcttgcacttaagaaaggcagccattacaatacagtcatcttacagaagactgatggtaaagaagaagttacaagaaatgcaaagggctgcagttctcattcaggctactttcaggatgtacagaacatatattacatttcagacttggaaacatgcttcaattctaattcagcaacattatcgaacatatagagctgcaaaattacaaagagaaaattatatcagacaatggcattctgctgtggttattcaggctgcatataaaggaatgaaagcaagacaacttttaagggaaaaacacaaagcttctatcgtaatacaaagcacctacagaatgtataggcagtattgtttctaccaaaagcttcagtgggctacaaaaatcatacaagaaaaatatagagcaaataaaaagaaacagaaagtatttcaacacaatgaacttaagaaagagacttgtgttcaggcaggttttcaggacatgaacataaaaaaacagattcaggaacagcaccaggctgccattattattcagaagcattgtaaagcctttaaaataaggaagcattatctccaccttagagcaacagtagtttctattcaaagaagatacagaaaactaactgcagtgcgtacccaagcagttatttgtatacagtcttattacagaggctttaaagtacgaaaggatattcaaaatatgcaccgggctgccacactaattcagtcattctatcgaatgcacagggccaaagttgattatgaaacaaagaaaactgcaattgtggttatacagaattattataggttgtatgttagagtaaaaacagaaagaaaaaactttttagcagttcagaaatctgtacgaactattcaggctgcttttagaggcatgaaagttagacaaaaattgaaaaatgtatcagaggaaaagatggcagccattgttaaccaatctgcactctgctgttacagaagtaaaactcagtatgaagctgttcaaagtgaaggtgttatgattcaagagtggtataaagcttctggccttgcttgttcacaggaagcagagtatcattctcaaagtagggctgcagtaacaattcaaaaagctttttgtagaatggtcacaagaaaactggaaacacagaaatgtgctgccctacggattcagttcttccttcagatggctgtgtatcggagaagatttgttcagcagaaaagagctgctatcactttacagcattattttaggacgtggcaaaccagaaaacagtttttactatatagaaaagcagcagtggttttacaaaatcactacagagcatttctgtctgcaaaacatcaaagacaagtctatttacagatcagaagcagtgttatcattattcaagctagaagtaaaggatttatacagaaacggaagtttcaggaaattaaaaatagcaccataaaaattcaggctatgtggaggagatatagagccaagaaatatttatgtaaagtgaaagctgcctgcaagattcaagcctggtatagatgttggagagcacacaaagaatatctagctatattaaaagctgttaaaattattcaaggttgcttctataccaaactagagagaacacggtttttgaatgtgagagcatcagcaattatcattcagagaaaatggagagctatacttcctgcaaagatagctcatgaacacttcttaatgataaaaagacatcgagctgcttgtttgatccaagcacattatagaggatataaaggaaggcaggtctttcttcggcagaaatctgctgctttgatcatacaaaaatatatacgagccagggaggctggaaagcatgaaaggataaaatatattgaatttaaaaaatctacagttatcctacaagcactggtgcgtggttggctagtacgaaaaagatttttagaacagagagccaaaattcgacttcttcacttcactgcagctgcatattatcacctgaatgctgttagaattcaaagagcctataaactttacctggctgtgaagaatgctaacaagcaggttaattcagtcatctgtattcagagatggtttcgagcaagattacaagaaaagagatttattcagaaatatcatagcatcaaaaagattgagcatgaaggtcaagaatgtctgagccagcgaaatagggctgcatcagtaatacagaaagcagtgcgccattttctcctccgtaaaaagcaggaaaaattcactagtggaatcattaaaattcaggcattatggagaggctattcttggaggaagaaaaatgattgtacaaaaattaaagctatacgactaagtcttcaagttgttaatagggagattcgagaagaaaacaaactctacaaaagaactgcacttgcacttcattaccttttgacatataagcacctttctgccattcttgaggccttaaaacacctagaggtagttactagattgtctccactttgttgtgagaacatggcccagagtggagcaatttctaaaatatttgttttgatccgaagttgtaatcgcagtattccttgtatggaagtcatcagatatgctgtgcaagtcttgcttaatgtatctaagtatgagaaaactacttcagcagtttatgatgtagaaaattgtatagatatactattggagcttttgcagatataccgagaaaagcctggtaataaagttgcagacaaaggcggaagcatttttacaaaaacttgttgtttgttggctattttactgaagacaacaaatagagcctctgatgtacgaagtaggtccaaagttgttgaccgtatttacagtctctacaaacttacagctcataaacataaaatgaatactgaaagaatactttacaagcaaaagaagaattcttctataagcattccttttatcccagaaacacctgtaaggaccagaatagtttcaagacttaagccagattgggttttgagaagagataacatggaagaaatcacaaatcccctgcaagctattcaaatggtgatggatacgcttggcattccttattagtaaatgtaaacattttcagtatgtatagtgtaaagaaatattaaagccaatcatgagtacgtaaagtgatttttgctctccgtgtacaacttttaaaatctgactttgttttaaaaaaacataaactgttcattacattcttcatttttatcatttatagttttatgcatgtaataaactaatatgtcataagatgaaaaaaaaaaaaaaaaaaa

SEQ ID NO: 71 - DNA, 6641 bp, Homo sapiens
ggttacattttggatcctcgcggagtactggtcaggcggttaagtcctgtacctaggaaagagggcgagctctggggcgctctccggtgtcatgaggagcttcaaaagagtcaactttgggactctgctaagcagccagaaggaggctgaagagttgctgcccgccttgaaggagttcctgtccaaccctccagctggttttcccagcagccgatctgatgctgagaggagacaagcttgtgatgccatcctgagggcttgcaaccagcagctgactgctaagctagcttgccctaggcatctggggagcctgctggagctggcagagctggcctgtgatggctacttagtgtctaccccacagcgtcctcccctctacctggaacgaattctctttgtcttactgcggaatgctgctgcacaaggaagcccagaggccacactccgccttgctcagcccctccatgcctgcttggtgcagtgctctcgcgaggctgctccccaggactatgaggccgtggctcggggcagcttttctctgctttggaagggggcagaagccctgttggaacggcgagctgcatttgcagctcggctgaaggccttgagcttcctagtactcttggaggatgaaagtaccccttgtgaggttcctcactttgcttctccaacagcctgtcgagcggtagctgcccatcagctatttgatgccagtggccatggtctaaatgaagcagatgctgatttcctagatgacctgctctccaggcacgtgatcagagccttggtgggtgagagagggagctcttctgggcttctttctccccagagggccctctgcctcttggagctcaccttggaacactgccgtcgcttttgctggagccgccaccatgacaaagccatcagcgcagtggagaaggctcacagttacctaaggaacaccaatctagcccctagccttcagctatgtcagctgggggttaagctgctgcaggttggggaggaaggacctcaggcagtggccaagcttctgatcaaggcatcagctgtcctgagcaagagtatggaggcaccatcacccccacttcgggcattgtatgagagctgccagttcttcctttcaggcctggaacgaggcaccaagaggcgctatagacttgatgccattctgagcctctttgcttttcttggagggtactgctctcttctgcagcagctgcgggatgatggtgtgtatgggggctcctccaagcaacagcagtcttttcttcagatgtactttcagggacttcacctctacactgtggtggtttatgactttgcccaaggctgtcagatagttgatttggctgacctgacccaactagtggacagttgtaaatctaccgttgtctggatgctggaggccttagagggcctgtcgggccaagagctgacggaccacatggggatgaccgcttcttacaccagtaatttggcctacagcttctatagtcacaagctctatgccgaggcctgtgccatctctgagccgctctgtcagcacctgggtttggtgaagccaggcacttatcccgaggtgcctcctgagaagttgcacaggtgcttccggctacaagtagagagtttgaagaaactgggtaaacaggcccagggctgcaagatggtgattttgtggctggcagccctgcaaccctgtagccctgaacacatggctgagccagtcactttctgggttcgggtcaagatggatgcggccagggctggagacaaggagctacagctaaagactctgcgagacagcctcagtggctgggacccggagaccctggccctcctgctgagggaggagctgcaggcctacaaggcggtgcgggccgacactggacaggaacgcttcaacatcatctgtgacctcctggagctgagccccgaggagacaccagccggggcctgggcacgagccacccacctggtagaactggctcaggtgctctgctaccacgactttacgcagcagaccaactgctctgctctggatgctatccgggaagccctgcagcttctggactctgtgaggcctgaggcccaggccagagatcagcttctggacgataaagcacaggccttgctgtggctttacatctgtactctggaagccaaaatgcaggaaggtatcgagcgggatcggagagcccaggcccctggtaacttggaggaatttgaagtcaatgacctgaactatgaagataaactccaggaagatcgtttcctatacagtaacattgccttcaacctggctgcagatgctgctcagtccaaatgcctggaccaagccctggccctgtggaaggagctgcttacaaaggggcaggccccagctgtacggtgtctccagcagacagcagcctcactgcagatcctagcagccctctaccagctggtggcaaagcccatgcaggctctggaggtcctcctgctgctacggattgtctctgagagactgaaggaccactcgaaggcagctggctcctcctgccacatcacccagctcctcctgaccctcggctgtcccagctatgcccagttacacctggaagaggcagcatcgagcctgaagcatctcgatcagactactgacacatacctgctcctttccctgacctgtgatctgcttcgaagtcaactctactggactcaccagaaggtgaccaagggtgtctctctgctgctgtctgtgcttcgggatcctgccctccagaagtcctccaaggcttggtacttgctgcgtgtccaggtcctgcagctggtggcagcttaccttagcctcccgtcaaacaacctctcacactccctgtgggagcagctctgtgcccaaggctggcagacacctgagatagctctcatagactcccataagctcctccgaagcatcatcctcctgctgatgggcagtgacattctctcaactcagaaagcagctgtggagacatcgtttttggactatggtgaaaatctggtacaaaaatggcaggttctttcagaggtgctgagctgctcagagaagctggtctgccacctgggccgcctgggtagtgtgagtgaagccaaggccttttgcttggaggccctaaaacttacaacaaagctgcagataccacgccagtgtgccctgttcctggtgctgaagggcgagctggagctggcccgcaatgacattgatctctgtcagtcggacctgcagcaggttctgttcttgcttgagtcttgcacagagtttggtggggtgactcagcacctggactctgtgaagaaggtccacctgcagaaggggaagcagcaggcccaggtcccctgtcctccacagctcccagaggaggagctcttcctaagaggccctgctctagagctggtggccactgtggccaaggagcctggccccatagcaccttctacaaactcctccccagtcttgaaaaccaagccccagcccatacccaacttcctgtcccattcacccacctgtgactgctcgctctgcgccagccctgtcctcacagcagtctgtctgcgctgggtattggtcacggcaggggtgaggctggccatgggccaccaagcccagggtctggatctgctgcaggtcgtgctgaagggctgtcctgaagccgctgagcgcctcacccaagctctccaagcttccctgaatcataaaacacccccctccttggttccaagcctcttggatgagatcttggctcaagcatacacactgttggcactggagggcctgaaccagccatcaaacgagagcctgcagaaggttctacagtcagggctgaagtttgtagcagcacggataccccacctagagccctggcgagccagcctgctcttgatttgggccctcacaaaactaggtggcctcagctgctgtactacccaactttttgcaagctcctggggctggcagccaccattaataaaaagtgtccctggctcagagccctctaagactcagggccaaaaacgttctggacgagggcgccaaaagttagcctctgctcccctgcgcctcaataatacctctcagaaaggtctggaaggtagaggactgccctgcacacctaaacccccagaccggatcaggcaagctggccctcatgtccccttcacggtgtttgaggaagtctgccctacagagagcaagcctgaagtaccccaggcccccagggtacaacagagagtccagacgcgcctcaaggtgaacttcagtgatgacagtgacttggaagaccctgtctcagctgaggcctggctggcagaggagcctaagagacggggcactgcttcccggggccgggggcgagcaaggaagggcctgagcctaaagacggatgccgtggttgccccaggtagtgcccctgggaaccctggcctgaatggcaggagccggagggccaagaaggtggcatcaagacattgtgaggagcggcgtccccagagggccagtgaccaggccaggcctggccctgagatcatgaggaccatccctgaggaagaactgactgacaactggagaaaaatgagctttgagatcctcaggggctctgacggggaagactcagcctcaggtgggaagactccagctccgggccctgaggcagcttctggagaatgggagctgctgaggctggattccagcaagaagaagctgcccagcccatgcccagacaaggagagtgacaaggaccttggtcctcggctccggctcccctcagcccccgtagccactggtctttctaccctggactccatctgtgactccctgagtgttgctttccggggcattagtcactgtcctcctagtgggctctatgcccacctctgccgcttcctggccttgtgcctgggccaccgggatccttatgccactgctttccttgtcaccgagtctgtctccatcacctgtcgccaccagctgctcacccacctccacagacagctcagcaaggcccagaagcaccgaggatcacttgaaatagcagaccagctgcaggggctgagccttcaggagatgcctggagatgtccccctggcccgcatccagcgcctcttttccttcagggctttggaatctggccacttcccccagcctgaaaaggagagtttccaggagcgcctggctctgatccccagtggggtgactgtgtgtgtgttggccctggccaccctccagcccggaaccgtgggcaacaccctcctgctgacccggctggaaaaggacagtcccccagtcagtgtgcagattcccactggccagaacaagcttcatctgcgttcagtcctgaatgagtttgatgccatccagaaggcacagaaagagaacagcagctgtactgacaagcgagaatggtggacagggcggctggcactggaccacaggatggaggttctcatcgcttccctagagaagtctgtgctgggctgctggaaggggctgctgctgccgtccagtgaggagcccggccctgcccaggaggcctcccgcctacaggagctgctacaggactgtggctggaaatatcctgaccgcactctgctgaaaatcatgctcagtggtgccggtgccctcacccctcaggacattcaggccctggcctacgggctgtgcccaacccagccagagcgagcccaggagctcctgaatgaggcagtaggacgtctacagggcctgacagtaccaagcaatagccaccttgtcttggtcctagacaaggacttgcagaagctgccgtgggaaagcatgcccagcctccaagcactgcctgtcacccggctgccctccttccgcttcctactcagctactccatcatcaaagagtatggggcctcgccagtgctgagtcaaggggtggatccacgaagtaccttctatgtcctgaaccctcacaataacctgtcaagcacagaggagcaatttcgagccaatttcagcagtgaagctggctggagaggagtggttggggaggtgccaagacctgaacaggtgcaggaagccctgacaaagcatgatttgtatatctatgcagggcatggggctggtgcccgcttccttgatgggcaggctgtcctgcggctgagctgtcgggcagtggccctgctgtttggctgtagcagtgcggccctggctgtgcgtggaaacctggagggggctggcatcgtgctcaagtacatcatggctggttgccccttgtttctgggtaatctctgggatgtgactgaccgcgacattgaccgctacacggaagctctgctgcaaggctggcttggagcaggcccaggggccccccttctctactatgtaaaccaggcccgccaagctccccgactcaagtatcttattggggctgcacctatagcctatggcttgcctgtctctctgcggtaaccccatggagctgtcttattgatgctagaagcctcataactgttctacctccaaggttagatttaatccttaggataactcttttaaagtgattttccccagtgttttatatgaaacatttccttttgatttaacctcagtataataaagatacatcatttaaaccctgaaaaaaaaaaaaaaaaaa

SEQ ID NO: 72 - DNA, 4045 bp, Homo sapiens
tcccggtgtgggtactgctgtctgtggtgtggctgtgggacccgtgagcaagcagcgacgccagcggcggagaaccgacgaaaggtgtcaccacagtgatggcagtggaggacagcacgctgcaagtagtggtacgggtgcggccccccacccctcgggagctggacagtcagcggcggccagtggttcaggtggtggacgagcgggtgctggtgtttaaccctgaggagcccgatggagggttccctggcctgaaatggggtggcacccatgatggccccaagaagaagggcaaagacctgacgtttgtctttgaccgggtctttggcgaggcggccacccaacaggacgtgttccagcacaccacgcacagcgtcctggacagcttcctccagggctacaactgctcagtgtttgcctacggggccaccggggctgggaagacacacaccatgctgggaagggagggggaccccggcatcatgtacctgaccaccgtggaactgtacaggcgcctggaggcccgccagcaggagaagcacttcgaggtgctcatcagctaccaggaggtgtataatgaacagatccatgacctcctggagcccaaggggccccttgccatccgcgaggaccccgacaagggggtggtggtgcaaggactttctttccaccagccagcctcagccgagcagctgctggagatactgaccagggggaaccgtaaccgcacgcagcaccccactgatgccaacgcgacttcctcccgctcccatgccatcttccagatctttgtgaagcagcaggaccgggttccaggactgacccaggctgtccaggtggccaagatgagcctgattgacctggctggctcagagcgggcatccagcacccatgcgaagggggagcggctgcgggagggggccaacatcaaccgctctctgctggcgctcatcaacgtcctcaatgccttggccgatgcaaagggccgcaagacccatgtgccctaccgggacagcaaactgacccgcctgctcaaagactccctcgggggcaactgccgcacagtgatgatcgctgccatcagcccctccagcctgacctacgaggacacgtacaacaccctcaaatatgccgaccgggccaaggagatcaggctctcgctgaagagcaatgtgaccagcctggactgtcacatcagccagtatgctaccatctgccaacagctccaggctgaggtagccgctctgaggaagaagctccaagtgtatgaggggggaggccagcccccaccacaggacctcccaggatctcccaagtcgggaccaccaccagaacaccagccctgcaccccagagctccctgcagggcctagagcccttcaagaggagagtctggggatggaggcccaggtggagagggccatggaagggaactcttcagaccaggagcagtccccagaggatgaggatgaaggcccagctgaggaggttccaacccagatgccagagcagaaccccacacatgcactgccagagtcccctcgcctgaccctgcagcccaagccagtcgtgggccacttctcagcacgggaactggatggggaccgttctaagcagttggccctaaaggtgctgtgcgttgcccagcggcagtactccctgctccaagcagccaacctcctgacgcccgacatgatcacagagtttgagaccctacagcagctggtgcaagaggaaaaaattgagcctggggcagaggccttgaggacttcaggcctggccaggggggcacctctggctcaggagctgtgttcagagtcaatccctgtgccgtctcctctctgcccagagcctccaggatacactggccctgtgacccggactatggcgaggcgactgagtggccccctgcacaccctgggaatcccgcctggacccaactgcaccccagcccaggggtcccgatggcccatggagaagaagaggaggagaccaagcgccttggaggcagacagtcccatggccccaaagcggggcaccaagcgccagcgccagtccttcctgccctgcctaaggagagggtctctgcctgacacccaaccttcacaggggcccagcacccccaaaggagaaagggcctcctccccctgccattcccctcgcgtttgcccagccacagtcatcaaaagccgggtgcccctgggcccttccgccatgcagaactgctccaccccgctggctctgcccactcgagacctcaatgccacctttgatctctctgaggagcctccctcaaagcccagtttccatgaatgcattggctgggacaaaataccccaggagctgagcaggctggaccagcccttcatccccagggcacctgtgcccctgttcaccatgaagggccccaagccaacatcttccctccctgggacctctgcctgcaagaagaagcgcgttgcgagttcctcagtctcccatggccgcagccgcatcgcccgcctccccagcagcactttgaagaggccagctgggccccttgtactcccagagctgcccttgagtcccctgtgccctagcaaccggaggaatggaaaggacctcatcagggtggggagagcactctcagcagggaacggcgtcaccaaggtgtcctgaccgccagaatgtcctgaccaccaaggtgtcctaacctaccggcccctctgctggatacccctcttggacctgtagccacctgcaccaggagctggacctgccttccttacctgggagcaattagtgccaacacacctttgctgtattaacatccctccccagacatccatcctgctactcaccctctgttaatctcctgttacactcagcttcttggcatgtacatattcatttgtgagtgttaatgtgctgctgttttttgttttttggtggtttttgttttttgttttttttgttttgagatggagtcttactctgtcgcccaggctggagtgcagtggtacgatcttggctcactgcaacctccgcctcctgggttcaagtaattctcctgcctcagctttccaagtagctgggattacaggcacccatcaccacacccagctaattttcgtctttttaatagagagggggtttttccatgttggccaggctggtcttgaactcctgacctcaggtgatccgcctgcctcagcttcccaaagtgctgagattacaggcatgagctaccacgcctggcccgtgttgctgttttaaaggtgctgccatgttcccccatcttttttttttttgagatggagtctcgctctgtcgcccaggctggagtgcagtggtggcgatcttggctcactgcaagctccgcctcccaggttcacaccattctcctgcctcagcctcccaagtagctgggactacaggcgcccaccaccacgcctggctaattttttgtatttttagtagagatggggtttcaccgtgttagccaggctggtctcgatctgacctcatgatccacccgcctcggcctcccaaagtgctgggattacaggcgtgagccactgcgcccggcctcccctctcatttatgatgccctctgtgcaggcagacggctcttgggctcttttccccacctgtctctaacacaggccccacggtgatggccacaggcagtagaggaggaatgaggatgggttggggagcggggagtcgcggcttggctcttcctggtttctgagagggacatcttcatccctactccccttggtccccaaccacagtcctggtgaagatgtggatgataatggtgccttgatttccaaatgaagacagctttattgcttaactctattgtacataggatacacgttcagtgtaaaataaagtgtaaaggggaattcaggcttaatgctgcacctagatataaatgctaatgatacttgggtttatagccttctgatcctttatttctgcatatatatatagatatatacatatatttttggtataacaataaaccgtctccatccttaaaaaaaaaaaaaaaaaaa

SEQ ID NO: 73 - DNA, 6069 bp, Homo sapiens
gtcctgtctggcggtgccgacggtgaggggcggtggcccaacggcgggagattcaaacctggaagaaggaggaacatggagaggagagcagcgggcccaggctgggcagcctatgaacgcctcacagctgaggagatggatgagcagaggcggcagaatgttgcctatcagtacctgtgccggctggaggaggccaagcgctggatggaggcctgcctgaaggaggagcttccttccccggtggagctggaggagagccttcggaatggagtgctgctggccaagctaggccactgttttgcaccctccgtggttcccttgaagaagatctacgatgtggagcagctgcggtaccaggcaactggcttacatttccgtcacacagacaacatcaacttttggctatctgcaatagcccacatcggtctgccttcgaccttcttcccagagaccacggacatctatgacaaaaagaacatgccccgggtagtctactgcatccatgctctcagtctcttcctcttccggctgggattggcccctcagatacatgatctatacgggaaagtgaaattcacagctgaggaactcagcaacatggcgtccgaactggccaaatatggcctccagctgcctgccttcagcaagatcgggggcatcttggccaatgagctctcggtggatgaggctgcagtccatgcagctgttcttgccatcaatgaagcagtggagcgaggggtggtggaggacaccctggctgccttgcagaatcccagtgctcttctggagaatctccgagagcctctggcagccgtctaccaagagatgctggcccaggccaagatggagaaggcagccaatgccaggaaccatgatgacagagaaagccaggacatctatgaccactacctaactcaggctgaaatccagggcaatatcaaccatgtcaacgtccatggggctctagaagttgttgatgatgccctggaaagacagagccctgaagccttgctcaaggcccttcaagaccctgccctggccctgcgaggggtgaggagagactttgctgactggtacctggagcagctgaactcagacagagagcagaaggcacaggagctgggcctggtggagcttctggaaaaggaggaagtccaggctggtgtggctgcagccaacacaaagggtgatcaggaacaagccatgctccacgctgtgcagcggatcaacaaagccatccggaggggagtggcggctgacactgtgaaggagctgatgtgccctgaggcccagctgcctccagtgtaccctgttgcatcgtctatgtaccagctggagctggcagtgctccagcagcagcagggggagcttggccaggaggagctcttcgtggctgtggagatgctctcagctgtggtcctgattaaccgggccctggaggcccgggatgccagtggcttctggagcagcctggtgaaccctgccacaggcctggctgaggtggaaggagaaaatgcccagcgttacttcgatgccctgctgaaattgcgacaggagcgtgggatgggtgaggacttcctgagctggaatgacctgcaggccaccgtgagccaggtcaatgcacagacccaggaagagactgaccgggtccttgcagtcagcctcatcaatgaggctctggacaaaggcagccctgagaagactctgtctgccctactgcttcctgcagctggcctagatgatgtcagcctccctgtcgcccctcggtaccatctcctccttgtggcagccaaaaggcagaaggcccaggtgacaggggatcctggagctgtgctgtggcttgaggagatccgccagggagtggtcagagccaaccaggacactaatacagctcagagaatggctcttggtgtggctgccatcaatcaagccatcaaggagggcaaggcagcccagactgagcgggtgttgaggaaccccgcagtggcccttcgaggggtagttcccgactgtgccaacggctaccagcgagccctggaaagtgccatggcaaagaaacagcgtccagcagacacagctttctgggttcaacatgacatgaaggatggcactgcctactacttccatctgcagaccttccaggggatctgggagcaacctcctggctgccccctcaacacctctcacctgacccgggaggagatccagtcagctgtcaccaaggtcactgctgcctatgaccgccaacagctctggaaagccaacgtcggctttgttatccagctccaggcccgcctccgtggcttcctagttcggcagaagtttgctgagcattcccactttctgaggacctggctcccagcagtcatcaagatccaggctcattggcggggttataggcagcggaagatttacctggagtggttgcagtattttaaagcaaacctggatgccataatcaagatccaggcctgggcccggatgtgggcagctcggaggcaatacctgaggcgtctgcactacttccagaagaatgttaactccattgtgaagatccaggcatttttccgagccaggaaagcccaagatgactacaggatattagtgcatgcaccccaccctcctctcagtgtggtacgcagatttgcccatctcttgaatcaaagccagcaagacttcttggctgaggcagagctgctgaagctccaggaagaggtagttaggaagatccgatccaatcagcagctggagcaggacctcaacatcatggacatcaagattggcctgctggtgaagaaccggatcactctgcaggaagtggtctcccactgcaagaagctgaccaagaggaataaggaacagctgtcagatatgatggttctggacaagcagaagggtttaaagtcgctgagcaaagagaaacggcagaaactagaagcataccaacacctcttctacctgctccagactcagcccatctacctggccaagctgatctttcagatgccacagaacaaaaccaccaagttcatggaggcagtgattttcagcctgtacaactatgcctccagccgccgagaggcctatctcctgctccagctgttcaagacagcactccaggaggaaatcaagtcaaaggtggagcagccccaggacgtggtgacaggcaacccaacagtggtgaggctggtggtgagattctaccgtaatgggcggggacagagtgccctgcaggagattctgggcaaggttatccaggatgtgctagaagacaaagtgctcagcgtccacacagaccctgtccacctctataagaactggatcaaccagactgaggcccagacagggcagcgcagccatctcccatatgatgtcaccccggagcaggccttgagccaccccgaggtccagagacgactggacatcgccctacgcaacctcctcgccatgactgataagttccttttagccatcacctcatctgtggaccaaattccgtatgggatgcgatatgtggccaaagtcctgaaggcaactctggcagagaaattccctgacgccacagacagcgaggtctataaggtggtcgggaacctcctgtactaccgcttcctgaacccagctgtggtggctcctgacgccttcgacattgtggccatggcagctggtggagccctggctgccccccagcgccatgccctgggggctgtggctcagctcctacagcacgctgcggctggcaaggccttctctgggcagagccagcacctacgggtcctgaatgactatctggaggaaacacacctcaagttcaggaagttcatccatagagcctgccaggtgccagagccagaggagcgttttgcagtggacgagtactcagacatggtggctgtggccaaacccatggtgtacatcaccgtgggggagctggtcaacacgcacaggctgttgctggagcaccaggactgcattgcccctgatcaccaagaccccctgcatgagctcctggaggatcttggggagctgcccaccatccctgaccttattggtgagagcatcgctgcagatgggcacacggacctgagcaagctagaagtgtccctgacgctgaccaacaagtttgaaggactagaggcagatgctgatgactccaacacccgtagcctgcttctgagcaccaagcagctgttggccgatatcatacagttccatcctggggacaccctcaaggagatcctgtccctctcggcttccagagagcaagaagcagcccacaagcagctgatgagccgacgccaggcctgtacagcccagacaccggagccactgcgacgacaccgctcactgacagctcactccctcctgccactggcagagaagcagcggcgcgtcctgcggaacctacgccgacttgaagccctggggttggtcagcgccagaaatggctaccaggggctagtggacgagctggccaaggacatccgcaaccagcacagacacaggcacaggcggaaggcagagctggtgaagctgcaggccacattacagggcctgagcactaagaccaccttctatgaggagcagggtgactactacagccagtacatccgggcctgcctggaccacctggcccccgactccaagagttctgggaaggggaagaagcagccttctcttcattacactgctgctcagctcctggaaaagggtgtcttggtggaaattgaagatcttcccgcctctcacttcagaaacgtcatctttgacatcacgccgggagatgaggcaggaaagtttgaagtaaatgccaagttcctgggtgtggacatggagcgatttcagcttcactatcaggatctcctgcagctccagtatgagggtgtggctgtcatgaaactcttcaacaaggccaaagtcaatgtcaaccttctcatcttcctcctcaacaagaagtttttgcggaagtgacagaggcaaagggtgctacccaagcccctcttacctctctggatgctttctttaacactaactcaccactgtgcttccctgcagacacccagagctcaggactgggcaaggcccagggattctcaccccttccccagctgggaggagcttgcctgcctggccacagacagtgtatcttctaattggctaaagtgggccttgcccagagtccagctgtgtggcttttatcatgcatgacaaacccctggctttcctgccagatggtaggacatggaccttgacctgggaaagccattactcttgtgtctgctactgccctcccacagtcaccccaatattacaagcactgccccagcggcttgatttcccctctgccttccttctctctgcactcccacaaagccagggccaggctccccatccctacctcccactgcatcagcagtgggtgttcctgcccttcctgagtctaggcagctctgctgctgtgatctgcacaccctccaacctgggcagggactggggggatgcagtgtgtgttagtgcccatgtggcattgtggcactgttgccccccatggcggcatgggcaagatgaccttccattagcttcaagtcttgttctcttgtctgtggtctgtttaatatgtgggtcactagggtatttattctttctcccatccttacactctggatcattgtgcagacttaatcagggttttaacgctttcatttttttttttttttttttttttttgagctcaaagagagttctcattttccctattcaaactaatacccatgccgtgttttttaccttggatttaaagtcaccttaggttggggcaacagattctcactcatgtttaagatcttgttatttcagcttcataagatcaaagaggagtctttcccttttctcttttaccctcaggattctcatcccttacagctgactcttccaggcaatttccatagatctgcagtcctgcctctgccacagtctctctgttgtccccacatctacccaacttcctgtactgttgcccttctgatgttaataaaagcagctgttactcccaaaaaaaaaaaaaaaaaa

SEQ ID NO: 74 - DNA, 2703 bp, Homo sapiens
gtcaatcagtaaagcatgaaatactatctcatgctcctggcaaggagcgactgtgacctgagaggaaaccaggaggcactgtcctcaatcccctgcctgcctcacagaccgtgggggcaccgtccgcatcacagtgtccaccgtccacaccgcctgtccaccgtccgcatcacagtgtccaccgtccacaccgcctgtccaccgtccgcatcacagtgtccaccgtccacaccgcctgtccaccgtccgcatctcagtgtccaccgtccacaccgcctgtccaccgtccgcatcacagtgtccaccgtccacaccgcctgtccaccgtccgcatcacagtgtccaccgtccacaccgcctgtccaccgtccgcatcacagtgtccaccgtccacactgcctgtccaccgtccacaccacctgtccaccatccacactgcagtgtccaccgtccacaccgcctgtccaccgtccgcatcacagtgtccaccgtccacaccgcctgtccaccgtccgcatcacagtgtccacagtccacactgcagtgtccaccgtccacaccacctgtccactgtcctcatcgcagtgtccaccgtcctcattgcagtgtccaccatccacccagcctgtccaccgtccacaccgcctgtccaccatccacaccacctgtccaccatccgcactgcagtgtccaccgtccacactgcctgtccaccgtccgcatcacagtgtccaccgtccacactgcctgtccaccgtccgcatcacagtgtccaccgtccacactgcctgtccaccgtccacaccacctgtccaccatccacactgcagtgtccactgtctacaccacctgtccactgtcctcatcgcagtgtccaccgtcctcatcgcagtgtccaccatccacccagcctgtccaccgtccacaccgcctgtccaccatccacactgcctgtccaccgtccgcatcacagtgtccaccgtccacactgcctgtccaccgtccacaccacctgtccaccatccacactgcagtgtccactgtccacaccacctgtccaccattcacactgcagtgtccaccgtccacaccacctgtccactgtcctcatcgcagtgtccaccgtcctcatcgcagtgtccaccatccacccagcctgtccaccgtccacaccgcctgtccaccatccacaccgcctgtccaccgtccgcatcacagtgtccaccgtccacaccgcctgtccaccgtccgcatcacagtgtccaccgtccacaccgcctgtccaccgtccgcatcacagtgtccaccgtccacaccgcctgtccaccgtccgcatcacagtgtccaccgtccacaccgcctgtccaccgtcctcatcacggtgtccaccgtccacaccgcctgtccaccgtccgcatcacggtgtccaccgtccacaccgcctgtccaccgtctgcatcacagtgtccaccgtccacactgcctgtccaccgtccacaccacctgtccaccatccacactgcagtgtccaccgtccacaccacctgtccactgtcctcatcgcagtgtccaccgtcctcatcgcagtgtccaccatccacccagcctgtccaccgtccacaccacctgtccaccatccacaccgcctgtccaccatctgcactgcagtgtccaccatccacaccacctgtccaccgtccacaccacctgtccatcatctgcactgcactgtctacctgctggcagcccgcagtgcctctggactctctgggccaggacaaactctaaatattgtccctcagatcaaatgcctcattttgggtatgaaaagcagggatccccctgtgtgtgatcagaaactgcacacagctaagccctcatacaaataaactctaaaatatcgctacatctgaagtggtcactggccactctgtgaacctgaagaggcctgtggatgctctctgacccacaccagagtgttggccttcttccgttcttcatggaggccttctgcaggcggggccctgggattttcagggcgctgggcgtttcttccatttatgtgtgtaagttttcattcttcttgattttttgaacccactcaagaatgatgggaaaatctgcccttatgatcatagtttctttaaaagttccagtctacttaattttgcagccataagactaagaagcgcacggaggaacaatagtggccactcagttcaggagacaggtccccagaggagatgcaggcaggcgagcatggcctctgtggggctctgtgctgtcagacgtctgggaggtggtgggcccctgacccgtatgtgccacccaaggccaggccaggcccagctccctcactgagccctgctccaaccaactccccagtgatgaccccactgctcagaggcaggtctgagaggggagggttagggcctcacatctgaggcccggcagaggctgccccgtccattcccctgccacgcgggcagaggcagcaggtggggccgacgagggggtggccaggcctgcccctgcccatccccagcaccgaccacagcagccccacctgggagggccaatctcttctggccactacggggacgacgtggtgagatttcccaagcgtgggaagggcagaggtgcccttggctcacctgatacacaggtgaggccccaccactgacttttcctcacagaaacagg